(19) United States
(12) Patent Application Publication
(10) Pub. No.: US 2016/0312292 A1
(43) Pub. Date: Oct. 27, 2016

(54) METHODS FOR TREATING HEMATOLOGICAL CANCERS AND THE USE OF BIOMARKERS AS A PREDICTOR OF CLINICAL SENSITIVITY TO IMMUNODULATORY THERAPIES

(71) Applicant: CELGENE CORPORATION, Summit, NJ (US)

(72) Inventor: Matthew William Burnell Trotter, Sevilla (ES)

(21) Appl. No.: 15/101,866

(22) PCT Filed: Dec. 5, 2014
(86) PCT No.: PCT/US14/68767

§ 371 Date: Jun. 3, 2016

Related U.S. Application Data
(60) Provisional application No. 61/913,046, filed on Dec. 6, 2013.

(51) Int. Cl. C12Q 1/68; A61K 31/454; G01N 33/574

(52) U.S. Cl. CPC C12Q 1/6886; G01N 33/57407; A61K 31/454; C12Q 2600/158; C12Q 2600/106

(57) ABSTRACT

Provided herein, in certain embodiments, are biomarkers for use in predicting the clinical sensitivity of hematologic cancers, such as non-Hodgkin's lymphoma, and a patient's response to treatment with an immunomodulatory agent, such as 3-(4-an-rino-1-oxo-3-dihydro-isoindol-2-y])-piperidine-2,6-dione, which is also known as lenalidomide or Revlimid®. Also provided herein, in certain embodiments, are methods of treating or managing non-Hodgkin's lymphomas, including but not limited to diffuse large B-cell lymphoma (DLBCL), using prognostic factors.

### CROSS-REFERENCE TO RELATED APPLICATIONS

This application claims the benefit of priority to U.S. Ser. No. 61/913,046 filed Dec. 6, 2013, which is herein incorporated by reference in its entirety.

### 1. FIELD

Provided herein are biomarkers for use in predicting the clinical sensitivity of hematologic cancers, such as non-Hodgkin's lymphoma, and a patient's response to treatment with an immunomodulatory agent, such as 3-(4-amino-1-oxo-1,3-dihydro-isoindol-2-yl)-piperidine-2,6-dione, which is also known as lenalidomide or Revlimid®. In one aspect, provided herein are methods of treating or managing non-Hodgkin's lymphomas, including but not limited to, diffuse large B-cell lymphoma (DLBCL), using prognostic factors.

### 2. BACKGROUND

### 2.1 Pathobiology of Cancer

Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites (metastasis). Clinical data and molecular biologic studies indicate that cancer is a multistep process that begins with minor pre-neoplastic changes, which may under certain conditions progress to neoplasia. The neoplastic lesion may evolve clonally and develop an increasing capacity for invasion, growth, metastasis, and heterogeneity, especially under conditions in which the neoplastic cells escape the host's immune surveillance. Roitt, I., Brostoff, J and Kale, D., Immunology, 17.1-17.12 (3rd ed., Mosby, St. Louis, Mo., 1993).

There is an enormous variety of cancers which are described in detail in the medical literature. Examples include cancers of the lung, colon, rectum, prostate, breast, brain, blood and intestine. The incidence of cancer continues to climb as the general population ages, as new cancers develop, and as susceptible populations (e.g., people infected with AIDS or excessively exposed to sunlight) grow. However, options for the treatment of cancer are limited. For example, in the case of blood cancers (e.g., multiple myeloma), few treatment options are available, especially when conventional chemotherapy fails and bone-marrow transplantation is not an option. A tremendous demand therefore exists for new methods and compositions that can be used to treat patients with cancer.

Many types of cancers are associated with new blood vessel formation, a process known as angiogenesis. Several of the mechanisms involved in tumor-induced angiogenesis have been elucidated. The most direct of these mechanisms is the secretion by the tumor cells of cytokines with angiogenic properties. Examples of these cytokines include acidic and basic fibroblastic growth factor (a,b-FGF), angiogenin, vascular endothelial growth factor (VEGF), and TNF-α. Alternatively, tumor cells can release angiogenic peptides through the production of proteases and the subsequent breakdown of the extracellular matrix where some cytokines are stored (e.g., b-FGF). Angiogenesis can also be induced indirectly through the recruitment of inflammatory cells (particularly macrophages) and their subsequent release of angiogenic cytokines (e.g., TNF-α, b-FGF).

Lymphoma refers to cancers that originate in the lymphatic system. Lymphoma is characterized by malignant neoplasms of lymphocytes—B lymphocytes and T lymphocytes (i.e., B-cells and T-cells). Lymphoma generally starts in lymph nodes or collections of lymphatic tissue in organs including, but not limited to, the stomach or intestines. Lymphoma may involve the marrow and the blood in some cases. Lymphoma may spread from one site to other parts of the body.

The treatment of various forms of lymphomas are described, for example, in U.S. Pat. No. 7,468,363, the entirety of which is incorporated herein by reference. Such lymphomas include, but are not limited to, Hodgkin's lymphoma, non-Hodgkin's lymphoma, cutaneous B-cell lymphoma, activated B-cell lymphoma, DLBCL, mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma and mantle zone lymphoma and low grade follicular lymphoma.

The non-Hodgkin lymphomas (NHLs) are a diverse group of blood cancers that include any kind of lymphoma except Hodgkin's lymphomas. Types of NHL vary significantly in their severity, from indolent to very aggressive. Less aggressive non-Hodgkin lymphomas are compatible with a long survival while more aggressive non-Hodgkin lymphomas can be rapidly fatal without treatment they can be formed from either B-cells or T-cells. B-cell non-Hodgkin lymphomas include Burkitt lymphoma, chronic lymphocytic leukemia/small lymphocytic lymphoma (CLL/SLL), diffuse large B-cell lymphoma, follicular lymphoma, immunoblastic large cell lymphoma, precursor B-lymphoblastic lymphoma, and mantle cell lymphoma. T-cell non-Hodgkin lymphomas include mycosis fungoides, anaplastic large cell lymphoma, and precursor T-lymphoblastic lymphoma. Prognosis and treatment depend on the stage and type of disease.

Diffuse large B-cell lymphoma (DLBCL) accounts for approximately one-third of non-Hodgkin's lymphomas. While some DLBCL patients are cured with traditional chemotherapy, the remainder die from the disease. Anticancer drugs cause rapid and persistent depletion of lymphocytes, possibly by direct apoptosis induction in mature T and B cells. See K. Stahnke. et al., Blood 2001, 98:3066-3073. Absolute lymphocyte count (ALC) has been shown to be a prognostic factor in follicular non-Hodgkin's lymphoma and recent results have suggested that ALC at diagnosis is an important prognostic factor in diffuse large B-cell lymphoma.

The diffuse large-B-cell lymphomas (DLBCL) can be divided into distinct molecular subtypes according to their gene profiling patterns: germinal-center B-cell-like DLBCL (GCB-DLBCL), activated B-cell-like DLBCL (ABC-DLBCL), and primary mediastinal B-cell lymphoma (PMBL) or unclassified type. These subtypes are characterized by distinct differences in survival, chemo-responsiveness, and signaling pathway dependence, particularly the NF-κB pathway. See D. Kim et al., Journal of Clinical Oncology, 2007 ASCO Annual Meeting Proceedings Part I. Vol 25, No. 18S (June 20 Supplement), 2007: 8082. See Bea S, et al., Blood 2005; 106: 3183-90; Ngo V. N. et al., Nature 2011; 470: 115-9. Such differences have prompted the search for more effective and subtype-specific treatment strategies in DLBCL.

Leukemia refers to malignant neoplasms of the blood-forming tissues. Various forms of leukemias are described, for example, in U.S. Pat. No. 7,393,862 and U.S. provisional patent application No. 60/380,842, filed May 17, 2002, the entireties of which are incorporated herein by reference. Although viruses reportedly cause several forms of leukemia in animals, causes of leukemia in humans are to a large extent unknown. The Merck Manual, 944-952 (17^{th} ed. 1999). Transformation to malignancy typically occurs in a single cell through two or more steps with subsequent proliferation and clonal expansion. In some leukemias, specific chromosomal translocations have been identified with consistent leukemic cell morphology and special clinical features (e.g., translocations of 9 and 22 in chronic myelocytic leukemia, and of 15 and 17 in acute promyelocytic leukemia). Acute leukemias are predominantly undifferentiated cell populations and chronic leukemias more mature cell forms.

Acute leukemias are divided into lymphoblastic (ALL) and non-lymphoblastic (ANLL) types. The Merck Manual, 946-949 (17^{th} ed. 1999). They may be further subdivided by their morphologic and cytochemical appearance according to the French-American-British (FAB) classification or according to their type and degree of differentiation. The use of specific B- and T-cell and myeloid-antigen monoclonal antibodies are most helpful for classification. ALL is predominantly a childhood disease which is established by laboratory findings and bone marrow examination. ANLL, also known as acute myelogenous leukemia or acute myeloid leukemia (AML), occurs at all ages and is the more common acute leukemia among adults; it is the form usually associated with irradiation as a causative agent.

Chronic leukemias are described as being lymphocytic (CLL) or myelocytic (CML). The Merck Manual, 949-952 (17^{th} ed. 1999). CLL is characterized by the appearance of mature lymphocytes in blood, bone marrow, and lymphoid organs. The hallmark of CLL is sustained, absolute lymphocytosis (>5,000/μL) and an increase of lymphocytes in the bone marrow. Most CLL patients also have clonal expansion of lymphocytes with B-cell characteristics. CLL is a disease of middle or old age. In CML, the characteristic feature is the predominance of granulocytic cells of all stages of differentiation in blood, bone marrow, liver, spleen, and other organs. In the symptomatic patient at diagnosis, the total white blood cell (WBC) count is usually about 200,000/μL, but may reach 1,000,000/μL. CML is relatively easy to diagnose because of the presence of the Philadelphia chromosome.

Bone marrow stromal cells are well known to support CLL disease progression and resistance to chemotherapy. Disrupting the interactions between CLL cells and stromal cells is an additional target of CLL chemotherapy.

In addition to the acute and chronic categorization, neoplasms are also categorized based upon the cells giving rise to such disorder into precursor or peripheral. See e.g., U.S. patent publication no. 2008/0051379, the disclosure of which is incorporated herein by reference in its entirety. Precursor neoplasms include ALLs and lymphoblastic lymphomas and occur in lymphocytes before they have differentiated into either a T- or B-cell. Peripheral neoplasms are those that occur in lymphocytes that have differentiated into either T- or B-cells. Such peripheral neoplasms include, but are not limited to, B-cell CLL, B-cell prolymphocytic leukemia, lymphoplasmacytic lymphoma, mantle cell lymphoma, follicular lymphoma, extranodal marginal zone B-cell lymphoma of mucosa-associated lymphoid tissue, nodal marginal zone lymphoma, splenic marginal zone lymphoma, hairy cell leukemia, plasmacytoma, diffuse large B-cell lymphoma and Burkitt lymphoma. In over 95 percent of CLL cases, the clonal expansion is of a B cell lineage. See Cancer: Principles & Practice of Oncology (3rd Edition) (1989) (pp. 1843-1847). In less than 5 percent of CLL cases, the tumor cells have a T-cell phenotype. Notwithstanding these classifications, however, the pathological impairment of normal hematopoiesis is the hallmark of all leukemias.

Multiple myeloma (MM) is a cancer of plasma cells in the bone marrow. Normally, plasma cells produce antibodies and play a key role in immune function. However, uncontrolled growth of these cells leads to bone pain and fractures, anemia, infections, and other complications. Multiple myeloma is the second most common hematological malignancy, although the exact causes of multiple myeloma remain unknown. Multiple myeloma causes high levels of proteins in the blood, urine, and organs, including but not limited to M-protein and other immunoglobulins (antibodies), albumin, and beta-2-microglobulin. M-protein, short for monoclonal protein, also known as paraprotein, is a particularly abnormal protein produced by the myeloma plasma cells and can be found in the blood or urine of almost all patients with multiple myeloma.

Skeletal symptoms, including bone pain, are among the most clinically significant symptoms of multiple myeloma. Malignant plasma cells release osteoclast stimulating factors (including IL-1, IL-6 and TNF) which cause calcium to be leached from bones causing lytic lesions; hypercalcemia is another symptom. The osteoclast stimulating factors, also referred to as cytokines, may prevent apoptosis, or death of myeloma cells. Fifty percent of patients have radiologically detectable myeloma-related skeletal lesions at diagnosis. Other common clinical symptoms for multiple myeloma include polyneuropathy, anemia, hyperviscosity, infections, and renal insufficiency.

Bone marrow stromal cells are well known to support multiple myeloma disease progression and resistance to chemotherapy. Disrupting the interactions between multiple myeloma cells and stromal cells is an additional target of multiple myeloma chemotherapy.

Further, rituximab is known to deplete normal host B cells. M. Aklilu et al., Annals of Oncology 15:1109-1114, 2004. The long-term immunologic effects of B cell depletion with rituximab and the characteristics of the reconstituting B cell pool in lymphoma patients are not well defined, despite the widespread usage of this therapy. See Jennifer H. Anolik et al., Clinical Immunology, vol. 122, issue 2, February 2007, pages 139-145.

The approach for patients with relapsed or refractory disease relies heavily on experimental treatments followed by stem cell transplantation, which may not be appropriate for patients with a poor performance status or advanced age. Therefore, a tremendous demand exists for new methods that can be used to treat patients with NHL.

The incidence of cancer continues to climb as the general population ages, as new cancers develop, and as susceptible populations (e.g., people infected with AIDS or excessively exposed to sunlight) grow. A tremendous demand therefore exists for new methods and compositions that can be used to treat patients with cancer including NHL.

### 2.2. Methods of Treatment

Current cancer therapy may involve surgery, chemotherapy, hormonal therapy and/or radiation treatment to eradicate neoplastic cells in a patient (see, for example, Stockdale, 1998, Medicine, vol. 3, Rubenstein and Federman, eds., Chapter 12, Section IV). Recently, cancer therapy could also involve biological therapy or immunotherapy. All of these approaches pose significant drawbacks for the patient. Surgery, for example, may be contraindicated due to the health of a patient or may be unacceptable to the patient. Additionally, surgery may not completely remove neoplastic tissue. Radiation therapy is only effective when the neoplastic tissue exhibits a higher sensitivity to radiation than normal tissue. Radiation therapy can also often elicit serious side effects. Hormonal therapy is rarely given as a single agent. Although hormonal therapy can be effective, it is often used to prevent or delay recurrence of cancer after other treatments have removed the majority of cancer cells. Biological therapies and immunotherapies are limited in number and may produce side effects such as rashes or swellings, flu-like symptoms, including fever, chills and fatigue, digestive tract problems or allergic reactions.

With respect to chemotherapy, there are a variety of chemotherapeutic agents available for treatment of cancer. A majority of cancer chemotherapeutics act by inhibiting DNA synthesis, either directly, or indirectly by inhibiting the biosynthesis of deoxyribonucleotide triphosphate precursors, to prevent DNA replication and concomitant cell division. Gilman et al., Goodman and Gilman's: The Pharmacological Basis of Therapeutics, Tenth Ed. (McGraw Hill, New York).

Despite availability of a variety of chemotherapeutic agents, chemotherapy has many drawbacks. Stockdale, Medicine, vol. 3. Rubenstein and Federman, eds., ch. 12, sect. 10, 1998. Almost all chemotherapeutic agents are toxic, and chemotherapy causes significant, and often dangerous side effects including severe nausea, bone marrow depression, and immunosuppression. Additionally, even with administration of combinations of chemotherapeutic agents, many tumor cells are resistant or develop resistance to the chemotherapeutic agents. In fact, those cells resistant to the particular chemotherapeutic agents used in the treatment protocol often prove to be resistant to other drugs, even if those agents act by different mechanism from those of the drugs used in the specific treatment. This phenomenon is referred to as pleiotropic drug or multidrug resistance. Because of the drug resistance, many cancers prove refractory to standard chemotherapeutic treatment protocols.

Still, there is a significant need for safe and effective methods of treating, preventing and managing cancer, particularly for tumors that are refractory to standard treatments, such as surgery, radiation therapy, chemotherapy and hormonal therapy, while reducing or avoiding the toxicities and/or side effects associated with the conventional therapies. Moreover, there remains a need for the ability to predict and monitor response to cancer therapy in order to increase the quality of care for cancer patients, avoid unnecessary treatment and to increase the success rate in cancer therapy in clinical practice.

### 3. SUMMARY

The present invention is based, in part, on the finding that certain genes are differentially expressed in DLBCL patients responsive to the immunomodulatory therapy lenalidomide (Revlimid®) relative to DLBCL patients unresponsive to lenalidomide. In addition, the present invention is based, in part, on the finding that the cellular composition (e.g., immune cell composition) of the tumor of a DLBCL patient may be indicative of whether the patient tumor will respond to an immunomodulatory therapy, such as lenalidomide, including its pharmaceutically acceptable salts, solvates or isomers.

In one aspect, provided herein are methods for predicting the clinical sensitivity of a hematological cancer to treatment with an immunomodulatory therapy comprising: (a) obtaining a first biological sample from a first patient having a hematological cancer, (b) measuring the level of expression of one, two, three, four, five or more of the genes identified in Table 3, infra, (c) comparing the level of expression of the one, two, three, four, five or more of the genes identified in Table 3 in the first biological sample with the level of expression of the same genes in a second biological sample from a second patient having the same type of hematological cancer as the first patient, wherein the hematological cancer in the second patient is clinically insensitive to an immunomodulatory therapy, and wherein the differential expression of the one, two, three, four, five or more of the genes in the first biological sample relative to the level of expression of the one, two, three, four, five or more of the genes in the second biological sample indicates that the hematological cancer in the first patient will be clinical sensitive to treatment with the immunomodulatory therapy. In a specific embodiment, the hematological cancer is DLBCL. In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. The immunomodulatory therapy can comprise the administration of an immunomodulatory compound, such as lenalidomide, or its pharmaceutically acceptable salts, solvates or isomers. An immunomodulatory therapy of the embodiments of the methods provided herein can comprise lenalidomide as immunomodulatory compound, or its pharmaceutically acceptable salts, solvates or isomers. In another specific embodiment, the immunomodulatory therapy is lenalidomide.

In another aspect, provided herein are methods for predicting the clinical sensitivity of a hematological cancer to treatment with an immunomodulatory therapy comprising: (a) obtaining a first biological sample from a first patient having a hematological cancer, (b) measuring the level of expression of one, two, three, four, five or more of the genes identified in Table 4, infra, (c) comparing the level of expression of the one, two, three, four, five or more of the genes identified in Table 4 in the first biological sample with the level of expression of the same genes in a second biological sample from a second patient having the same type of hematological cancer as the first patient, wherein the hematological cancer in the second patient is clinically insensitive to an immunomodulatory therapy, and wherein the differential expression of the one, two, three, four, five or more of the genes in the first biological sample relative to the level of expression of the one, two, three, four, five or more of the genes in the second biological sample indicates that the hematological cancer in the first patient will be clinical sensitive to treatment with the immunomodulatory therapy. In a specific embodiment, the hematological cancer is DLBCL. In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. The immunomodulatory therapy can comprise the administration of an immunomodulatory compound, such as lenalidomide, or its pharmaceutically acceptable salts, solvates or isomers. An immunomodulatory therapy of the embodiments of the methods provided herein can comprise lenalidomide as immunomodulatory compound, or its pharmaceutically acceptable salts, solvates or isomers. In another specific embodiment, the immunomodulatory therapy is lenalidomide.

In another aspect, provided herein are methods for predicting the clinical sensitivity of a hematological cancer to treatment with an immunomodulatory therapy comprising: (a) obtaining a first biological sample from a first patient having a hematological cancer, (b) measuring the level of expression of one, two, three, four, five or more of the genes identified in Table 1, infra, and (c) comparing the level of expression of the one, two, three, four, five or more of the genes identified in Table 1 in the first biological sample with the level of expression of the same genes in a second biological sample from a second patient having the same type of hematological cancer as the first patient, wherein the hematological cancer in the second patient is clinically insensitive to the immunomodulatory therapy, and wherein a higher level of expression of the one, two, three, four, five or more of the genes in the first biological sample relative to the level of expression of the one, two, three, four, five or more of the genes in the second biological sample indicates that the hematological cancer in the first patient will be clinical sensitive to treatment with the immunomodulatory therapy. In a specific embodiment, the hematological cancer is DLBCL. In certain embodiments, the DLBCL is refractory to certain therapies, such as chemotherapy. In some embodiments, the DLBCL is refractory relapsed in a patient. In a specific embodiment, the DLBCL is an activated B-cell-like subtype. In another specific embodiment, the DLBCL is a germinal center B-cell-like subtype. The immunomodulatory therapy can comprise the administration of an immunomodulatory compound, such as lenalidomide, or its pharmaceutically acceptable salts, solvates or isomers. An immunomodulatory therapy of the embodiments of the methods provided herein can comprise lenalidomide as immunomodulatory compound, or its pharmaceutically acceptable salts, solvates or isomers. In another specific embodiment, the immunomodulatory therapy is lenalidomide.

In another aspect, provided herein are methods for predicting the clinical sensitivity of a hematological cancer to treatment with an immunomodulatory therapy comprising: (a) obtaining a first biological sample from a first patient having a hematological cancer, (b) measuring the level of expression of one, two, three, four, five or more of the genes identified in Table 2, infra, and (c) comparing the level of expression of the one, two, three, four, five or more of the genes identified in Table 2 in the first biological sample with the level of expression of the same genes in a second biological sample is from a second patient having the same type of hematological cancer as the first patient, wherein the hematological cancer in the second patient is clinically insensitive to the immunomodulatory therapy, and wherein a lower level of expression of the one, two, three, four, five or more of the genes in the first biological sample relative to the level of expression of the one, two, three, four, five or more of the genes in the second biological sample indicates that the hematological cancer in the first patient will be clinical sensitive to treatment with the immunomodulatory therapy. In a specific embodiment, the hematological cancer is DLBCL. In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. The immunomodulatory therapy can comprise the administration of an immunomodulatory compound, such as lenalidomide, or its pharmaceutically acceptable salts, solvates or isomers. An immunomodulatory therapy of the embodiments of the methods provided herein can comprise lenalidomide as immunomodulatory compound, or its pharmaceutically acceptable salts, solvates or isomers. In another specific embodiment, the immunomodulatory therapy is lenalidomide.

In another aspect, provided herein are methods for predicting the clinical sensitivity of a hematological cancer to treatment with an immunomodulatory therapy comprising: (a) obtaining a first biological sample from a first patient having a hematological cancer, (b) measuring the level of expression of one, two, three, four, five or more of the genes identified in Table 1, infra, and measuring the level of expression one, two, three, four, five or more of the genes identified in Table 2, infra, and (c) comparing the level of expression of the genes identified in Tables 1 and 2 in the first biological sample with the level of expression of the same genes in a second biological sample is from a second patient having the same type of hematological cancer as the first patient, wherein the hematological cancer in the second patient is clinically insensitive to the immunomodulatory therapy, and wherein (i) a higher level of expression of the one, two, three, four, five or more of the genes identified in Table 1 in the first biological sample relative to the level of expression of the one, two, three, four, five or more of the genes in the second biological sample, and (ii) a lower level of expression of the one, two, three, four, five or more of the genes identified in Table 2 in the first biological sample relative to the level of expression of the one, two, three, four, five or more of the genes in the second biological sample, indicates that the hematological cancer in the first patient will be clinical sensitive to treatment with the immunomodulatory therapy. In a specific embodiment, the hematological cancer is DLBCL. In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. In another specific embodiment, the immunomodulatory therapy is lenalidomide.

In another aspect, provided herein are methods for predicting the clinical sensitivity of a hematological cancer to treatment with an immunomodulatory therapy comprising: (a) obtaining a first biological sample from a first patient having a hematological cancer, (b) measuring the expression of the genes or a certain subset of genes set forth in Table 1, 2, 3 or 4, or any combination thereof in the first biological sample, and (c) comparing the gene expression profile of the genes or subset of genes in the first biological sample to (i) the gene expression profile of the genes or subset of genes in tumor samples from patients having the same type of hematological cancer which are clinically sensitive to an immunomodulatory therapy and (ii) the gene expression of the genes or subset of genes in tumor samples from patients having the same type of hematological cancer which are clinically insensitive to the immunomodulatory therapy, wherein a gene expression profile for the genes or subset of genes in the first biological sample similar to the gene expression profile for the genes or subset of genes in tumor samples from patients having the same type of hematological cancer which are clinically sensitive to the immunomodulatory therapy indicates that the hematological cancer in the first patient will be clinical sensitive to treatment with the immunomodulatory therapy, and a gene expression profile for the genes or subset of genes in first biological sample similar to the gene expression profile for the genes or subset of genes in tumor samples from patients having the same type of hematological cancer which are clinically insensitive to the immunomodulatory therapy indicates that the hematological cancer of the first patient will be clinically insensitive to the treatment with the immunomodulatory therapy. In certain embodiments, the subset of genes comprises 3, 4, 5, 6, 7, 8, 9, 10, 11, 12, 14, 15 or more of the genes in Table 1, 2, 3 or 4, or any combination thereof. In some embodiments, the subset of genes comprises 2-5, 5-10, 10-15, 15-20, 20-25 or 25-30 of the genes in Table 1, 2, 3 or 4, or any combination thereof. In a specific embodiment, the hematological cancer is DLBCL. In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. The immunomodulatory therapy can comprise the administration of an immunomodulatory compound, such as lenalidomide, or its pharmaceutically acceptable salts, solvates or isomers. An immunomodulatory therapy of the embodiments of the methods provided herein can comprise lenalidomide as immunomodulatory compound, or its pharmaceutically acceptable salts, solvates or isomers. In another specific embodiment, the immunomodulatory therapy is lenalidomide.

In another aspect, provided herein are methods for predicting the clinical sensitivity of a hematological cancer to treatment with an immunomodulatory therapy comprising: (a) obtaining a first tumor sample from a first patient having the hematological cancer, (b) measuring the proportion of dendritic cells in the first tumor sample, and (c) comparing the proportion of dendritic cells in the first tumor sample with the proportion of dendritic cells in a second tumor sample from a second patient having the same type of hematological cancer, wherein the second patient's hematological cancer is clinically insensitive to treatment with the immunomodulatory therapy, and wherein a higher proportion of dendritic cells in the first tumor sample relative the proportion of dendritic cells in the second tumor sample indicates that the hematological cancer in the first patient will be clinical sensitive to treatment with the immunomodulatory therapy. In a specific embodiment, the hematological cancer is DLBCL. In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. The immunomodulatory therapy can comprise the administration of an immunomodulatory compound, such as lenalidomide, or its pharmaceutically acceptable salts, solvates or isomers. An immunomodulatory therapy of the embodiments of the methods provided herein can comprise lenalidomide as immunomodulatory compound, or its pharmaceutically acceptable salts, solvates or isomers. In another specific embodiment, the immunomodulatory therapy is lenalidomide.

In another aspect, provided herein are methods for predicting the clinical sensitivity of a hematological cancer to treatment with an immunomodulatory therapy comprising: (a) obtaining a first tumor sample from a first patient having the hematological cancer, (b) measuring the proportion of plasma cells in the first tumor sample, and (c) comparing the proportion of plasma cells in the first tumor sample with the proportion of plasma cells in a second tumor sample from a second patient having the same type of hematological cancer, wherein the second patient's hematological cancer is clinically insensitive to treatment with the immunomodulatory therapy, and wherein a higher proportion of plasma cells in the first tumor sample relative the proportion of plasma cells in the second tumor sample indicates that the hematological cancer in the first patient will be clinical sensitive to treatment with the immunomodulatory therapy. In a specific embodiment, the hematological cancer is DLBCL. In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. The immunomodulatory therapy can comprise the administration of an immunomodulatory compound, such as lenalidomide, or its pharmaceutically acceptable salts, solvates or isomers. An immunomodulatory therapy of the embodiments of the methods provided herein can comprise lenalidomide as immunomodulatory compound, or its pharmaceutically acceptable salts, solvates or isomers. In another specific embodiment, the immunomodulatory therapy is lenalidomide.

In another aspect, provided herein are methods for predicting the clinical sensitivity of a hematological cancer to treatment with an immunomodulatory therapy comprising: (a) obtaining a first tumor sample from a first patient having the hematological cancer, (b) measuring the proportion of dendritic cells and plasma cells in the first tumor sample, and (c) comparing the proportion of dendritic cells and plasma cells in the first tumor sample with the proportion of dendritic cells and plasma cells in a second tumor sample from a second patient having the same type of hematological cancer, wherein the second patient's hematological cancer is clinically insensitive to treatment with the immunomodulatory therapy, and wherein a higher proportion of dendritic cells and plasma cells in the first tumor sample relative the proportion of dendritic cells and plasma cells in the second tumor sample indicates that the hematological cancer in the first patient will be clinical sensitive to treatment with the immunomodulatory therapy. In a specific embodiment, the hematological cancer is DLBCL. In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. The immunomodulatory therapy can comprise the administration of an immunomodulatory compound, such as lenalidomide, or its pharmaceutically acceptable salts, solvates or isomers. An immunomodulatory therapy of the embodiments of the methods provided herein can comprise lenalidomide as immunomodulatory compound, or its pharmaceutically acceptable salts, solvates or isomers. In another specific embodiment, the immunomodulatory therapy is lenalidomide.

In another aspect, provided herein are methods for predicting the clinical sensitivity of a hematological cancer to treatment with an immunomodulatory therapy comprising: (a) obtaining a first tumor sample from a first patient having a hematological cancer, (b) measuring the proportion of immune cells in the tumor sample, and (c) comparing the proportion of the immune cells in the first tumor sample to (i) the proportion of the same immune cells in tumor samples from patients having the same type of hematological cancer which are clinically sensitive to an immunomodulatory therapy and (ii) the proportion of the same immune cells in tumor samples from patients having the same type of hematological cancer which are clinically insensitive to the immunomodulatory therapy, wherein a proportion of the immune cells in the first tumor sample similar to the proportion of the same immune cells in tumor samples from patients having the same type of hematological cancer which are clinically sensitive to the immunomodulatory therapy indicates that the hematological cancer in the first patient will be clinical sensitive to treatment with the immunomodulatory therapy, and a proportion of the immune cells in the first tumor sample similar to the proportion of the same immune cells in tumor samples from patients having the same type of hematological cancer which are clinically insensitive to the immunomodulatory therapy indicates that the hematological cancer in the first patient will be clinical insensitive to treatment with the immunomodulatory therapy. In some embodiments, the immune cells are subset of immune cells, such as subset of B cells. In certain embodiments, the immune cells are dendritic cells. In some embodiments, the immune cells are plasma cells. In certain embodiments, the immune cells are monocytes. In some embodiments, the immune cells are tumor infiltrating immune cells. In certain embodiments, the immune cells are T cells. In some embodiments, the immune cells are B cells. In certain embodiments, the immune cells are NK cells. In some embodiments, the immune cells are two, three or more subsets of immune cells, such as two more types of T cells (e.g., CD4+ and CD8+ T cells). In some embodiments, the proportion of different populations of immune cells in the first tumor sample are compared to (i) the proportion of the same populations of immune cells in the tumor samples from patients having the same type of hematological cancer which are clinically sensitive to the immunomodulatory therapy and (ii) the proportion of the same populations of immune cells in the tumor samples from patients having the same type of hematological cancer which are clinically insensitive to the immunomodulatory therapy. In a specific embodiment, the hematological cancer is DLBCL. In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. The immunomodulatory therapy can comprise the administration of an immunomodulatory compound, such as lenalidomide, or its pharmaceutically acceptable salts, solvates or isomers. An immunomodulatory therapy of the embodiments of the methods provided herein can comprise lenalidomide as immunomodulatory compound, or its pharmaceutically acceptable salts, solvates or isomers. In another specific embodiment, the immunomodulatory therapy is lenalidomide.

In another aspect, provided herein are methods for managing or treating a hematological cancer comprising: (a) obtaining a first biological sample from a first patient having a hematological cancer, (b) measuring the level of expression of one, two, three, four, five or more of the genes identified in Table 3, infra, (c) comparing the level of expression of the one, two, three, four, five or more of the genes identified in Table 3 in the first biological sample with the level of expression of the same genes in a second biological sample from a second patient having the same type of hematological cancer as the first patient, wherein the hematological cancer in the second patient is clinically insensitive to an immunomodulatory therapy, and (d) administering the immunomodulatory therapy to the first patient if the one, two, three, four, five or more of the genes in the first biological sample are differentially expressed relative to the level of expression of the one, two, three, four, five or more of the genes in the second biological sample. In a specific embodiment, the hematological cancer is DLBCL. In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. The immunomodulatory therapy can comprise the administration of an immunomodulatory compound, such as lenalidomide, or its pharmaceutically acceptable salts, solvates or isomers. An immunomodulatory therapy of the embodiments of the methods provided herein can comprise lenalidomide as immunomodulatory compound, or its pharmaceutically acceptable salts, solvates or isomers. In another specific embodiment, the immunomodulatory therapy is lenalidomide.

In another aspect, provided herein are methods for managing or treating a hematological cancer comprising: (a) obtaining a first biological sample from a first patient having a hematological cancer, (b) measuring the level of expression of one, two, three, four, five or more of the genes identified in Table 4, infra, (c) comparing the level of expression of the one, two, three, four, five or more of the genes identified in Table 4 in the first biological sample with the level of expression of the same genes in a second biological sample from a second patient having the same type of hematological cancer as the first patient, wherein the hematological cancer in the second patient is clinically insensitive to an immunomodulatory therapy, and (d) administering the immunomodulatory therapy to the first patient if the one, two, three, four, five or more of the genes in the first biological sample are differentially expressed relative to the level of expression of the one, two, three, four, five or more of the genes in the second biological sample. In a specific embodiment, the hematological cancer is DLBCL. In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. The immunomodulatory therapy can comprise the administration of an immunomodulatory compound, such as lenalidomide, or its pharmaceutically acceptable salts, solvates or isomers. An immunomodulatory therapy of the embodiments of the methods provided herein can comprise lenalidomide as immunomodulatory compound, or its pharmaceutically acceptable salts, solvates or isomers. In another specific embodiment, the immunomodulatory therapy is lenalidomide.

In another aspect, provided herein are methods for managing or treating a hematological cancer comprising: (a) obtaining a first biological sample from a first patient having a hematological cancer, (b) measuring the level of expression of one, two, three, four, five or more of the genes identified in Table 1, infra, (c) comparing the level of expression of the one, two, three, four, five or more of the genes identified in Table 1 in the first biological sample with the level of expression of the same genes in a second biological sample from a second patient having the same type of hematological cancer as the first patient, wherein the hematological cancer in the second patient is clinically insensitive to an immunomodulatory therapy, and (d) administering the immunomodulatory therapy to the first patient if a higher level of expression of the one, two, three, four, five or more of the genes in the first biological sample is measured relative to the level of expression of the one, two, three, four, five or more of the genes in the second biological sample. In a specific embodiment, the hematological cancer is DLBCL. In certain embodiments, the DLBCL is refractory to certain therapies, such as chemotherapy. In some embodiments, the DLBCL is relapsed in a patient. In a specific embodiment, the DLBCL is an activated B-cell-like subtype. The immunomodulatory therapy can comprise the administration of an immunomodulatory compound, such as lenalidomide, or its pharmaceutically acceptable salts, solvates or isomers. An immunomodulatory therapy of the embodiments of the methods provided herein can comprise lenalidomide as immunomodulatory compound, or its pharmaceutically acceptable salts, solvates or isomers. In another specific embodiment, the immunomodulatory therapy is lenalidomide.

In another aspect, provided herein are methods for managing or treating a hematological cancer comprising: (a) obtaining a first biological sample from a first patient having a hematological cancer, (b) measuring the level of expression of one, two, three, four, five or more of the genes identified in Table 2, infra, (c) comparing the level of expression of the one, two, three, four, five or more of the genes identified in Table 2 in the first biological sample with the level of expression of the same genes in a second biological sample is from a second patient having the same type of hematological cancer as the first patient, wherein the hematological cancer in the second patient is clinically insensitive to an immunomodulatory therapy, and (d) administering the immunomodulatory therapy to the first patient if a lower level of expression of the one, two, three, four, five or more of the genes in the first biological sample is measured relative to the level of expression of the one, two, three, four, five or more of the genes in the second biological sample. In certain embodiments, the immunomodulatory therapy is not administered or additional assays are conducted if the level of expression of one, two, three, four, five or more of the genes are not lower in the first biological sample than in the second biological sample. In a specific embodiment, the hematological cancer is DLBCL. In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. The immunomodulatory therapy can comprise the administration of an immunomodulatory compound, such as lenalidomide, or its pharmaceutically acceptable salts, solvates or isomers. An immunomodulatory therapy of the embodiments of the methods provided herein can comprise lenalidomide as immunomodulatory compound, or its pharmaceutically acceptable salts, solvates or isomers. In another specific embodiment, the immunomodulatory therapy is lenalidomide.

In another aspect, provided herein are methods for managing or treating a hematological cancer comprising: (a) obtaining a first biological sample from a first patient having a hematological cancer, (b) measuring the level of expression of one, two, three, four, five or more of the genes identified in Table 1, infra, and measuring the level of expression one, two, three, four, five or more of the genes identified in Table 2, supra, (c) comparing the level of expression of the genes identified in Tables 1 and 2 in the first biological sample with the level of expression of the same genes in a second biological sample is from a second patient having the same type of hematological cancer as the first patient, wherein the hematological cancer in the second patient is clinically insensitive to an immunomodulatory therapy, and (d) administering the immunomodulatory to the first patient if (i) a higher level of expression of the one, two, three, four, five or more of the genes identified in Table 1 in the first biological sample is measured relative to the level of expression of the one, two, three, four, five or more of the genes in the second biological sample, and (ii) a lower level of expression of the one, two, three, four, five or more of the genes identified in Table 2 in the first biological sample is measured relative to the level of expression of the one, two, three, four, five or more of the genes in the second biological sample. In a specific embodiment, the hematological cancer is DLBCL. In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. The immunomodulatory therapy can comprise the administration of an immunomodulatory compound, such as lenalidomide, or its pharmaceutically acceptable salts, solvates or isomers. An immunomodulatory therapy of the embodiments of the methods provided herein can comprise lenalidomide as immunomodulatory compound, or its pharmaceutically acceptable salts, solvates or isomers. In another specific embodiment, the immunomodulatory therapy is lenalidomide.

In another aspect, provided herein are methods for managing or treating a hematological cancer comprising: (a) obtaining a first biological sample from a first patient having a hematological cancer, (b) measuring the expression of a certain subset of genes set forth in Table 1, 2, 3 or 4, or any combination thereof in the first biological sample, and (c) comparing the gene expression profile of the subset of genes in the first biological sample to (i) the gene expression profile of the subset of genes in tumor samples from patients having the same type of hematological cancer which are clinically sensitive to an immunomodulatory therapy and (ii) the gene expression of the subset of genes in tumor samples from patients having the same type of hematological cancer which are clinically insensitive to the immunomodulatory therapy, and (d) administering the immunomodulatory therapy to the first patient if: (i) the gene expression profile for the subset of genes in the first biological sample is similar to the gene expression profile for the subset of genes in tumor samples from patients having the same type of hematological cancer which are clinically sensitive to the immunomodulatory therapy and (ii) the gene expression profile for the subset of genes in first biological sample is not similar to the gene expression profile for the subset of genes in tumor samples from patients having the same type of hematological cancer which are clinically insensitive to the immunomodulatory therapy. In certain embodiments, the subset of genes comprises 3, 4, 5, 6, 7, 8, 9, 10, 11, 12, 14, 15 or more of the genes in Table 1, 2, 3 or 4, or any combination thereof. In some embodiments, the subset of genes comprises 2-5, 5-10, 10-15, 15-20, 20-25 or 25-30 of the genes in Table 1, 2, 3 or 4, or any combination thereof. In a specific embodiment, the hematological cancer is DLBCL. In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. In another specific embodiment, the immunomodulatory therapy is lenalidomide.

In another aspect, provided herein are methods for managing or treating a hematological cancer comprising: (a) obtaining a first tumor sample from a first patient having the hematological cancer, (b) measuring the proportion of dendritic cells in the first tumor sample, (c) comparing the proportion of dendritic cells in the first tumor sample with the proportion of dendritic cells in a second tumor sample from a second patient having the same type of hematological cancer, wherein the second patient's hematological cancer is clinically insensitive to treatment with an immunomodulatory therapy, and (d) administering the immunomodulatory therapy to the first patient if a higher proportion of dendritic cells in the first tumor sample is measured relative the proportion of dendritic cells in the second tumor sample. In a specific embodiment, the hematological cancer is DLBCL. In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. The immunomodulatory therapy can comprise the administration of an immunomodulatory compound, such as lenalidomide, or its pharmaceutically acceptable salts, solvates or isomers. An immunomodulatory therapy of the embodiments of the methods provided herein can comprise lenalidomide as immunomodulatory compound, or its pharmaceutically acceptable salts, solvates or isomers. In another specific embodiment, the immunomodulatory therapy is lenalidomide.

In another aspect, provided herein are methods for managing or treating a hematological cancer comprising: (a) obtaining a first tumor sample from a first patient having the hematological cancer, (b) measuring the proportion of plasma cells in the first tumor sample, (c) comparing the proportion of plasma cells in the first tumor sample with the proportion of plasma cells in a second tumor sample from a second patient having the same type of hematological cancer, wherein the second patient's hematological cancer is clinically insensitive to treatment with an immunomodulatory therapy, and (d) administering the immunomodulatory therapy to the first patient if a higher proportion of plasma cells in the first tumor sample is measured relative the proportion of plasma cells in the second tumor sample. In a specific embodiment, the hematological cancer is DLBCL. In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. The immunomodulatory therapy can comprise the administration of an immunomodulatory compound, such as lenalidomide, or its pharmaceutically acceptable salts, solvates or isomers. An immunomodulatory therapy of the embodiments of the methods provided herein can comprise lenalidomide as immunomodulatory compound, or its pharmaceutically acceptable salts, solvates or isomers. In another specific embodiment, the immunomodulatory therapy is lenalidomide.

In another aspect, provided herein are methods for managing or treating a hematological cancer comprising: (a) obtaining a first tumor sample from a first patient having the hematological cancer, (b) measuring the proportion of dendritic cells and plasma cells in the first tumor sample, (c) comparing the proportion of dendritic cells and plasma cells in the first tumor sample with the proportion of dendritic cells and plasma cells in a second tumor sample from a second patient having the same type of hematological cancer, wherein the second patient's hematological cancer is clinically insensitive to treatment with an immunomodulatory therapy, and (d) administering the immunomodulatory therapy to the first patient if a higher proportion of dendritic cells and plasma cells in the first tumor sample is measured relative the proportion of dendritic cells and plasma cells in the second tumor sample. In a specific embodiment, the hematological cancer is DLBCL. In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. The immunomodulatory therapy can comprise the administration of an immunomodulatory compound, such as lenalidomide, or its pharmaceutically acceptable salts, solvates or isomers. An immunomodulatory therapy of the embodiments of the methods provided herein can comprise lenalidomide as immunomodulatory compound, or its pharmaceutically acceptable salts, solvates or isomers. In another specific embodiment, the immunomodulatory therapy is lenalidomide.

In another aspect, provided herein are for managing or treating a hematological cancer comprising: (a) obtaining a first tumor sample from a first patient having a hematological cancer, (b) measuring the proportion of immune cells in the first tumor sample, and (c) comparing the proportion of the immune cells in the first tumor sample to (i) the proportion of the same immune cells in tumor samples from patients having the same type of hematological cancer which are clinically sensitive to an immunomodulatory therapy and (ii) the proportion of the same immune cells in tumor samples from patients having the same type of hematological cancer which are clinically insensitive to the immunomodulatory therapy, and (d) administering the immunomodulatory therapy to the first patient if the proportion of the immune cells in the first tumor sample is (i) similar to the proportion of the same immune cells in tumor samples from patients having the same type of hematological cancer which are clinically sensitive to the immunomodulatory therapy, and (ii) not similar to the proportion of the same immune cells in tumor samples from patients having the same type of hematological cancer which are clinically insensitive to the immunomodulatory therapy. In some embodiments, the immune cells are subset of immune cells, such as subset of B cells. In certain embodiments, the immune cells are dendritic cells. In some embodiments, the immune cells are plasma cells. In certain embodiments, the immune cells are monocytes. In some embodiments, the immune cells are tumor infiltrating immune cells. In certain embodiments, the immune cells are T cells. In some embodiments, the immune cells are B cells. In certain embodiments, the immune cells are NK cells. In some embodiments, the immune cells are two, three or more subsets of immune cells, such as two more types of T cells (e.g., CD4+ and CD8+ T cells). In some embodiments, the proportion of different populations of immune cells in the first tumor sample are compared to (i) the proportion of the same populations of immune cells in the tumor samples from patients having the same type of hematological cancer which are clinically sensitive to the immunomodulatory therapy and (ii) the proportion of the same populations of immune cells in the tumor samples from patients having the same type of hematological cancer which are clinically insensitive to the immunomodulatory therapy. In a specific embodiment, the hematological cancer is DLBCL. In certain embodiments, the DLBCL is refractory to certain therapies, such as chemotherapy. In some embodiments, the DLBCL is relapsed in a patient. In a specific embodiment, the DLBCL is an activated B-cell-like subtype. In another specific embodiment, the DLBCL is a germinal center B-cell-like subtype. The immunomodulatory therapy can comprise the administration of an immunomodulatory compound, such as lenalidomide, or its pharmaceutically acceptable salts, solvates or isomers. An immunomodulatory therapy of the embodiments of the methods provided herein can comprise lenalidomide as immunomodulatory compound, or its pharmaceutically acceptable salts, solvates or isomers. In another specific embodiment, the immunomodulatory therapy is lenalidomide.

In accordance with the methods described herein, the biological sample can be any sample obtained from the patient. In certain embodiments, the biological sample is a cell sample. In other embodiments, the biological sample is whole blood sample, peripheral blood mononuclear cell sample, or tissue sample. In specific embodiments, the biological sample is a tumor sample. See Section 5.8, infra, regarding biological samples.

In accordance with the methods described herein, the level of expression of one, two, three, four, five or more of the genes in Table 1 and/or Table 2 and/or Table 3 and/or Table 4, infra, can be measured at the RNA and/or protein levels. In certain embodiments, the level of expression of the genes are measured at the RNA (e.g., mRNA) level. In other embodiments, the level of expression of the genes are measured at the protein level.

In another aspect, provided herein are kits useful for predicting the likelihood of an effective patient tumor response. In certain embodiments, the kit comprises a solid support, and a means for detecting the protein expression of at least one biomarker in a biological sample. Such a kit may employ, for example, a dipstick, a membrane, a chip, a disk, a test strip, a filter, a microsphere, a slide, a multiwell plate, or an optical fiber. The solid support of the kit can be, for example, a plastic, silicon, a metal, a resin, glass, a membrane, a particle, a precipitate, a gel, a polymer, a sheet, a sphere, a polysaccharide, a capillary, a film, a plate, or a slide. In some embodiments, the kit comprises a solid support, nucleic acids contacting the support, where the nucleic acids are complementary to at least 20, 50, 100, 200, 350, or more bases of mRNA, and a means for detecting the expression of the mRNA in a biological sample.

In certain embodiments, the kits provided herein employ means for detecting the expression of a biomarker by quantitative real-time PCR (QRT-PCR), microarray, flow cytometry or immunofluorescence. In other embodiments, the expression of the biomarker is measured by ELISA-based methodologies or other similar methods known in the art.

### 4. BRIEF DESCRIPTION OF THE FIGURES

FIG. 1: Hierarchical clustering (Euclidean distance; Ward linkage) of relative gene expression across 21 lenalidomide/Revlimid®-arm FF profiles, as represented by A. 1018 genes deemed significantly differentially regulated at FDR5%, and B. A subset of those genes deemed significantly differentially regulated at FDR1%, between discrete best-response categories. Gene expressions standardized to zero mean and unit standard variance across all profiles. Bars below dendrogram display: DLBCL cell of origin sub-type {GCB (white), ABC/Other (black), as determined by IHC at screen}; Investigator defined best response of patients in the Revlimid arm, {CR,PR,SD}(black) vs. {PD,Death}(white).

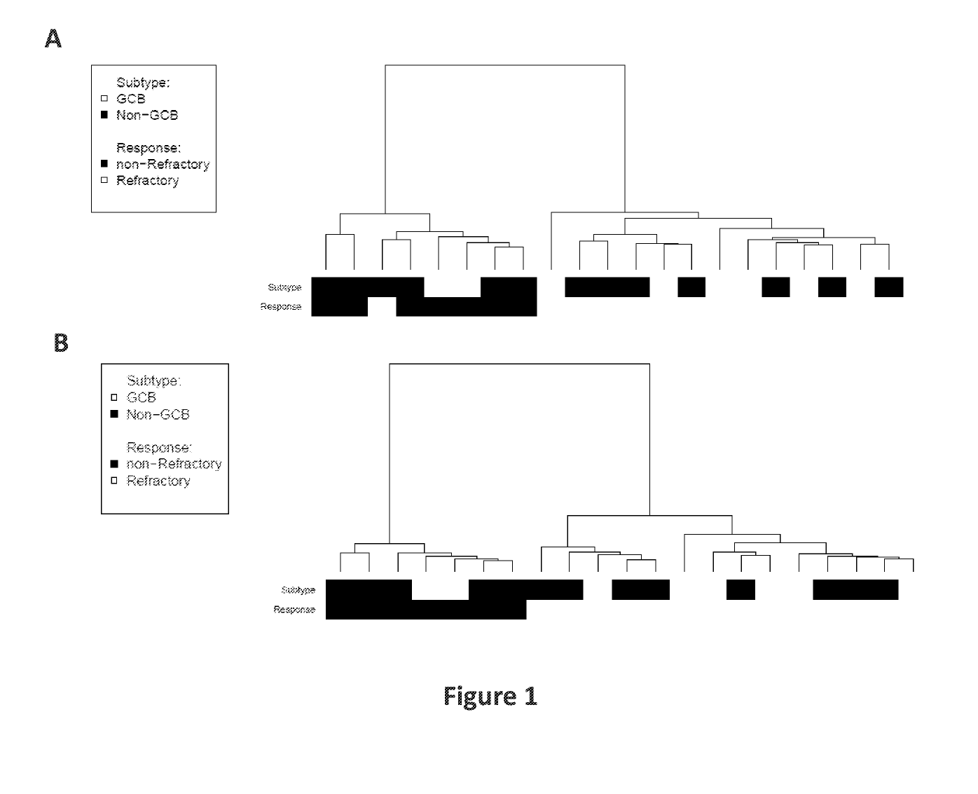

FIG. 2: Decomposition of 21 lenalidomide/Revlimid®-arm profiles derived from FF samples. Each boxplot represents estimated proportion (y-axis) of corresponding cell phenotype (x-axis) across two discrete Investigator defined best-response categories, {CR,PR,SD} (grey) and {PD,death}(white). Cell-types on the x-axis are: T-helper cells (Th); Activated T-helper cells (Th act); T-cells (Tc); Activated T-cells (Tc act); B-cells (B); Activated B-cells (B act); BCR-ligated B-cells (B aIgM); IgG Memory B-cells (Mem IgG); IgM Memory B-cells (Mem IgM); Plasma cells (PC); Natural Killer cells (NK); Activated Natural Killer cells (NK act); Monocytes (mono); Activated Monocytes (mono act); Dendritic Cells (DC); Activated Dendritic cells (DC act); Neutrophils (neutro). Phenotypic cell types defined in (Abbas et al., PLoS One, 2009).

FIG. 3: Summed estimated proportion of resting and activated dendritic cells (y-axis, left) across 21 lenalidomide/Revlimid®-arm profiles derived from FF samples (x-axis; triangles, ordered by descending PFS). PFS (y-axis, right; unit weeks) overlaid as line-connected points, with censor events denoted by a cross.

FIG. 4: Summed estimated proportion of BCR-ligated B-cells (y-axis, left) across 21 lenalidomide/Revlimid®-arm profiles derived from FF samples (x-axis; triangles, ordered by descending PFS). PFS (y-axis, right; unit weeks) overlaid as line-connected points, with censor events denoted by a cross.

FIG. 5: Bar plot of difference in estimated proportion of BCR-ligated B-cells and plasma cells (y-axis, left), derived from lenalidomide/Revlimid®-arm FF profiles (one profile per bar, x-axis; sorted in order of increasing difference between BCR-ligated B-cell/plasma-cell proportions. PFS (y-axis, right; unit weeks) overlaid as line-connected points, with censor events denoted by a cross. Dashed line represents median PFS in the two groups defined by estimated BCR-ligated B-cell proportion being greater or less than estimated plasma cell proportion.

### 5. DETAILED DESCRIPTION

### 5.1 Terminology

As used herein, and unless otherwise specified, the terms “treat,” “treating” and “treatment” refer to an action that occurs while a patient is suffering from the specified cancer, which includes the reduction in the severity of the cancer, reduces tumor size, or retards or slows the progression of the cancer.

The term “sensitivity” and “sensitive” when made in reference to treatment with compound is a relative term which refers to the degree of effectiveness of the compound in lessening or decreasing the progress of a tumor or the disease being treated.

As used herein, and unless otherwise specified, the term “effective amount” of a compound is an amount sufficient to provide a therapeutic benefit in the treatment or management of a cancer, or to delay or minimize one or more symptoms associated with the presence of the cancer. An effective amount of a compound means an amount of therapeutic agent, alone or in combination with other therapies, which provides a therapeutic benefit in the treatment or management of the cancer. The term “effective amount” can encompass an amount that improves overall therapy, reduces or avoids symptoms or causes of cancer, or enhances the therapeutic efficacy of another therapeutic agent.

As used herein, an “effective patient tumor response” refers to any increase in the therapeutic benefit to the patient. An “effective patient tumor response” can be, for example, a 5%, 10%, 15%, 20%, 25%, 30%, 40%, 50%, 60%, 70%, 80%, 90%, or 100% decrease in the rate of progress of the tumor. An “effective patient tumor response” can be, for example, a 5%, 10%, 15%, 20%, 25%, 30%, 40%, 50%, 60%, 70%, 80%, 90%, or 100% decrease in the physical symptoms of a cancer. An “effective patient tumor response” can be, for example, a 5%, 10%, 15%, 20%, 25%, 30%, 40%, 50%, 60%, 70%, 80%, 90%, or 100% decrease in the size of a tumor. An “effective patient tumor response” can be, for example, a 5%, 10%, 15%, 20%, 25%, 30%, 40%, 50%, 60%, 70%, 80%, 90%, or 100% decrease in the physical symptoms of a cancer. An “effective patient tumor response” can also be, for example, a 5%, 10%, 15%, 20%, 25%, 30%, 40%, 50%, 60%, 70%, 80%, 90%, 100%, or 200%, or more increase in the response of the patient, as measured by any suitable means, such as gene expression, cell counts, assay results, etc.

The term “likelihood” generally refers to an increase in the probability of an event. The term “likelihood” when used in reference to the effectiveness of a patient tumor response generally contemplates an increased probability that the rate of tumor progress or tumor cell growth will decrease. The term “likelihood” when used in reference to the effectiveness of a patient tumor response can also generally mean the increase of indicators, such as mRNA or protein expression, that may evidence an increase in the progress in treating the tumor.

The term “predict” generally means to determine or tell in advance. When used to “predict” the effectiveness of a cancer treatment, for example, the term “predict” can mean that the likelihood of the outcome of the cancer treatment can be determined at the outset, before the treatment has begun, or before the treatment period has progressed substantially.

An improvement in the cancer or cancer-related disease can be characterized as a complete or partial response. “Complete response” refers to an essential absence (or absence) of clinically detectable disease with normalization of any previously abnormal radiographic studies, bone marrow, and cerebrospinal fluid (CSF) or abnormal monoclonal protein measurements. “Partial response” refers to at least about a 5%, 10%, 15%, 20%, 25%, 30%, 40%, 50%, 60%, 70%, 80%, or 90% decrease in all measurable tumor burden (i.e., the number of malignant cells present in the subject, or the measured bulk of tumor masses or the quantity of abnormal monoclonal protein) in the absence of new lesions. The term “treatment” contemplates both a complete and a partial response.

“Tumor,” as used herein, refers to all neoplastic cell growth and proliferation, whether malignant or benign, and all pre-cancerous and cancerous cells and tissues. “Neoplastic,” as used herein, refers to any form of dysregulated or unregulated cell growth, whether malignant or benign, resulting in abnormal tissue growth. Thus, “neoplastic cells” include malignant and benign cells having dysregulated or unregulated cell growth.

The terms “cancer” and “cancerous” refer to or describe the physiological condition in mammals that is typically characterized by unregulated cell growth. Examples of cancer include, but are not limited to, blood-borne tumors (e.g., multiple myeloma, lymphoma and leukemia), and solid tumors.

The term “refractory or resistant” refers to a circumstance where patients, even after intensive treatment, have residual cancer cells (e.g., leukemia or lymphoma cells) in their lymphatic system, blood and/or blood forming tissues (e.g., marrow).

As used herein the terms “polypeptide” and “protein” as used interchangeably herein, refer to a polymer of amino acids of three or more amino acids in a serial array, linked through peptide bonds. The term “polypeptide” includes proteins, protein fragments, protein analogues, oligopeptides and the like. The term polypeptide as used herein can also refer to a peptide. The amino acids making up the polypeptide may be naturally derived, or may be synthetic. The polypeptide can be purified from a biological sample.

An mRNA that is “upregulated” is generally increased upon a given treatment or condition. An mRNA that is “downregulated” generally refers to a decrease in the level of expression of the mRNA in response to a given treatment or condition. In some situations, the mRNA level can remain unchanged upon a given treatment or condition.

An mRNA from a patient sample can be “upregulated” when treated with an immunomodulatory therapy, as compared to a control. This upregulation can be, for example, an increase of about 5%, 10%, 20%, 30%, 40%, 50%, 60%, 70%, 90%, 100%, 200%, 300%, 500%, 600%, 700%, 800%, 900%, 1,000%, 1,500%, 2,000%, 2,500%, 3,00%, 3,500%, 4,000%, 4,500%, 5,000% or more of the comparative control mRNA level.

Alternatively, an mRNA can be “downregulated”, or expressed at a lower level, in response to administration of certain immunomodulatory therapies or other therapies. A downregulated mRNA can be, for example, present at a level of about 99%, 95%, 90%, 85%, 80%, 75%, 70%, 65%, 60%, 55%, 50%, 45%, 40%, 35%, 30%, 25%, 20%, 15%, 10%, 5%, 3%, 1% or less of the comparative control mRNA level.

Similarly, the level of a polypeptide or protein biomarker from a patient sample can be increased when treated with an immunomodulatory therapy, as compared to a non-treated control. This increase can be about 5%, 10%, 20%, 30%, 40%, 50%, 60%, 70%, 80%, 90%, 100%, 200%, 300%, 400%, 500%, 700%, 1,000%, 1,500%, 2,000%, 2,500%, 3,000%, 3,500%, 4,000%, 4,500%, 5,000% or more of the comparative control protein level.

Alternatively, the level of a protein biomarker can be decreased in response to administration of certain immunomodulatory therapies or other agents. This decrease can be, for example, present at a level of about 99%, 95%, 90%, 80%, 70%, 60%, 50%, 40%, 30%, 20%, 10%, 5%, 3%, 1% or less of the comparative control protein level.

The terms “determining”, “measuring”, “evaluating”, “assessing” and “assaying” as used herein generally refer to any form of measurement, and include determining if an element is present or not. These terms include both quantitative and/or qualitative determinations. Assessing may be relative or absolute. “Assessing the presence of” can include determining the amount of something present, as well as determining whether it is present or absent.

The terms “nucleic acid” and “polynucleotide” are used interchangeably herein to describe a polymer of any length composed of nucleotides, e.g., deoxyribonucleotides or ribonucleotides, or compounds produced synthetically, which can hybridize with naturally occurring nucleic acids in a sequence specific manner analogous to that of two naturally occurring nucleic acids, e.g., can participate in Watson-Crick base pairing interactions. As used herein in the context of a polynucleotide sequence, the term “bases” (or “base”) is synonymous with “nucleotides” (or “nucleotide”), i.e., the monomer subunit of a polynucleotide. The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. “Analogues” refer to molecules having structural features that are recognized in the literature as being mimetics, derivatives, having analogous structures, or other like terms, and include, for example, polynucleotides incorporating non-natural nucleotides, nucleotide mimetics such as 2′-modified nucleosides, peptide nucleic acids, oligomeric nucleoside phosphonates, and any polynucleotide that has added substituent groups, such as protecting groups or linking moieties.

The terms “isolated” and “purified” refer to isolation of a substance (such as mRNA or protein) such that the substance comprises a substantial portion of the sample in which it resides, i.e. greater than the substance is typically found in its natural or un-isolated state. Typically, a substantial portion of the sample comprises, e.g., greater than 1%, greater than 2%, greater than 5%, greater than 10%, greater than 20%, greater than 30%, greater than 50%, or more, usually up to about 90%-100% of the sample. For example, a sample of isolated mRNA can typically comprise at least about 1% total mRNA. Techniques for purifying polynucleotides are well known in the art and include, for example, gel electrophoresis, ion-exchange chromatography, affinity chromatography, flow sorting, and sedimentation according to density.

The term “sample” as used herein relates to a material or mixture of materials, typically, although not necessarily, in fluid form, containing one or more components of interest.

“Biological sample” as used herein refers to a sample obtained from a biological subject, including sample of biological tissue or fluid origin, obtained, reached, or collected in vivo or in situ. A biological sample also includes samples from a region of a biological subject containing precancerous or cancer cells or tissues. Such samples can be, but are not limited to, organs, tissues, fractions and cells isolated from a subject. Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like. Preferred biological samples include but are not limited to whole blood, partially purified blood, PBMCs, tissue biopsies, and the like.

As used herein, the terms “patient” and “subject” refer to an animal, such as a mammal. In a specific embodiment, the patient is a human. In other embodiments, the patient is a non-human animal, such as a dog, cat, farm animal (e.g., horse, pig, or donkey), chimpanzee, or monkey.

A biological marker or “biomarker” is a substance whose detection indicates a particular biological state, such as, for example, the presence of cancer. In some embodiments, biomarkers can either be determined individually, or several biomarkers can be measured simultaneously.

A “biomarker” can indicate a change in the level of mRNA expression that may correlate with the risk or progression of a disease, or with the susceptibility of the disease to a given treatment. In some embodiments, the biomarker is a nucleic acid, such as a mRNA or cDNA.

A “biomarker” can also indicate a change in the level of polypeptide or protein expression that may correlate with the risk, susceptibility to treatment, or progression of a disease. The biomarker can be a polypeptide or protein, or a fragment thereof. The relative level of specific proteins can be determined by methods known in the art. For example, antibody based methods, such as an immunoblot, enzyme-linked immunosorbent assay (ELISA), or other methods can be used.

As used herein and unless otherwise indicated, the term “pharmaceutically acceptable salt” encompasses non-toxic acid and base addition salts of the compound to which the term refers. Acceptable non-toxic acid addition salts include those derived from organic and inorganic acids or bases know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

As used herein and unless otherwise indicated, the term “solvate” means a compound provided herein or a salt thereof, that further includes a stoichiometric or non-stoichiometric amount of solvent bound by non-covalent intermolecular forces. Where the solvent is water, the solvate is a hydrate.

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. As used herein and unless otherwise indicated, the term “stereomerically enriched” means a composition that comprises greater than about 60% by weight of one stereoisomer of a compound, preferably greater than about 70% by weight, more preferably greater than about 80% by weight of one stereoisomer of a compound. As used herein and unless otherwise indicated, the term “enantiomerically pure” means a stereomerically pure composition of a compound having one chiral center. Similarly, the term “stereomerically enriched” means a stereomerically enriched composition of a compound having one chiral center.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

The practice of the embodiments provided herein will employ, unless otherwise indicated, conventional techniques of molecular biology, microbiology, and immunology, which are within the skill of those working in the art. Such techniques are explained fully in the literature. Examples of particularly suitable texts for consultation include the following: Sambrook et al. (1989) Molecular Cloning; A Laboratory Manual (2d ed.); D. N. Glover, ed. (1985) DNA Cloning, Volumes I and II; M. J. Gait, ed. (1984) Oligonucleotide Synthesis; B. D. Hames & S J. Higgins, eds. (1984) Nucleic Acid Hybridization; B. D. Hames & S. J. Higgins, eds. (1984) Transcription and Translation; R. I. Freshney, ed. (1986) Animal Cell Culture; Immobilized Cells and Enzymes (IRL Press, 1986); Immunochemical Methods in Cell and Molecular Biology (Academic Press, London); Scopes (1987) Protein Purification: Principles and Practice (2d ed.; Springer Verlag, N.Y.); and D. M. Weir and C. C. Blackwell, eds. (1986) Handbook of Experimental Immunology, Volumes I-IV.

### 5.2 Methods for Predicting Clinical Sensitivity of a Hematological Cancer by Measuring Gene Expression

In one aspect, provided herein are methods for predicting the clinical sensitivity of a hematological cancer to treatment with an immunomodulatory therapy comprising: (a) obtaining a first biological sample from a first patient having a hematological cancer, (b) measuring the level of expression of one, two, three, four, five or more of the genes identified in Table 3 or 4, infra, (c) comparing the level of expression of the one, two, three, four, five or more of the genes identified in Table 3 or 4 in the first biological sample with the level of expression of the same genes in a second biological sample from a second patient having the same type of hematological cancer as the first patient, wherein the hematological cancer in the second patient is clinically insensitive to an immunomodulatory therapy, and wherein the differential expression of the one, two, three, four, five or more of the genes in the first biological sample relative to the level of expression of the one, two, three, four, five or more of the genes in the second biological sample indicates that the hematological cancer in the first patient will be clinical sensitive to treatment with the immunomodulatory therapy.

In another aspect, provided herein are methods for predicting the clinical sensitivity of a hematological cancer to treatment with an immunomodulatory therapy comprising: (a) obtaining a first biological sample from a first patient having a hematological cancer, (b) measuring the level of expression of one, two, three, four, five or more of the genes identified in Table 1, infra, and (c) comparing the level of expression of the one, two, three, four, five or more of the genes identified in Table 1 in the first biological sample with the level of expression of the same genes in a second biological sample from a second patient having the same type of hematological cancer as the first patient, wherein the hematological cancer in the second patient is clinically insensitive to the immunomodulatory therapy, and wherein a higher level of expression of the one, two, three, four, five or more of the genes in the first biological sample relative to the level of expression of the one, two, three, four, five or more of the genes in the second biological sample indicates that the hematological cancer in the first patient will be clinical sensitive to treatment with the immunomodulatory therapy.

In another aspect, provided herein are methods for predicting the clinical sensitivity of a hematological cancer to treatment with an immunomodulatory therapy comprising: (a) obtaining a first biological sample from a first patient having a hematological cancer, (b) measuring the level of expression of one, two, three, four, five or more of the genes identified in Table 2, infra, and (c) comparing the level of expression of the one, two, three, four, five or more of the genes identified in Table 2 in the first biological sample with the level of expression of the same genes in a second biological sample is from a second patient having the same type of hematological cancer as the first patient, wherein the hematological cancer in the second patient is clinically insensitive to the immunomodulatory therapy, and wherein a lower level of expression of the one, two, three, four, five or more of the genes in the first biological sample relative to the level of expression of the one, two, three, four, five or more of the genes in the second biological sample indicates that the hematological cancer in the first patient will be clinical sensitive to treatment with the immunomodulatory therapy.

In another aspect, provided herein are methods for predicting the clinical sensitivity of a hematological cancer to treatment with an immunomodulatory therapy comprising: (a) obtaining a first biological sample from a first patient having a hematological cancer, (b) measuring the level of expression of one, two, three, four, five or more of the genes identified in Table 1, infra, and measuring the level of expression one, two, three, four, five or more of the genes identified in Table 2, infra, and (c) comparing the level of expression of the genes identified in Tables 1 and 2 in the first biological sample with the level of expression of the same genes in a second biological sample is from a second patient having the same type of hematological cancer as the first patient, wherein the hematological cancer in the second patient is clinically insensitive to the immunomodulatory therapy, and wherein (i) a higher level of expression of the one, two, three, four, five or more of the genes identified in Table 1 in the first biological sample relative to the level of expression of the one, two, three, four, five or more of the genes in the second biological sample, and (ii) a lower level of expression of the one, two, three, four, five or more of the genes identified in Table 2 in the first biological sample relative to the level of expression of the one, two, three, four, five or more of the genes in the second biological sample, indicates that the hematological cancer in the first patient will be clinical sensitive to treatment with the immunomodulatory therapy.

In another aspect, provided herein are methods for predicting the clinical sensitivity of a hematological cancer to treatment with an immunomodulatory therapy comprising: (a) obtaining biological samples from patients having a hematological cancer, (b) measuring the level of expression of one, two, three, four, five or more of the genes identified in Table 3, infra, (c) assessing expression levels of the selected genes, either individually, conjointly, or via a functional transformation thereof, and (d) using of the expression levels to predict patients as sensitive or insensitive to an immunomodulatory therapy, via similarity to expression phenotypes displayed across the same genes by patients with the same indication and already known to be sensitive or insensitive to that therapy.

In one aspect, provided herein are methods for predicting the clinical sensitivity of a hematological cancer to treatment with an immunomodulatory therapy comprising: (a) obtaining biological samples from patients having a hematological cancer, (b) measuring the level of expression of one, two, three, four, five or more of the genes identified in Table 4, infra, (c) assessing expression levels of the selected genes, either individually, conjointly, or via a functional transformation thereof, and (d) using of the expression levels to predict patients as sensitive or insensitive to an immunomodulatory therapy, via similarity to expression phenotypes displayed across the same genes by patients with the same indication and already known to be sensitive or insensitive to that therapy.

In one aspect, provided herein are methods for predicting the clinical sensitivity of a hematological cancer to treatment with an immunomodulatory therapy comprising: (a) obtaining biological samples from patients having a hematological cancer, (b) measuring the level of expression of one, two, three, four, five or more of the genes identified in Table 1, infra, (c) assessing expression levels of the selected genes, either individually, conjointly, or via a functional transformation thereof, and (d) using of the expression levels to predict patients as sensitive or insensitive to an immunomodulatory therapy, via similarity to expression phenotypes displayed across the same genes by patients with the same indication and already known to be sensitive or insensitive to that therapy.

In one aspect, provided herein are methods for predicting the clinical sensitivity of a hematological cancer to treatment with an immunomodulatory therapy comprising: (a) obtaining biological samples from patients having a hematological cancer, (b) measuring the level of expression of one, two, three, four, five or more of the genes identified in Table 2, infra, (c) assessing expression levels of the selected genes, either individually, conjointly, or via a functional transformation thereof, and (d) using of the expression levels to predict patients as sensitive or insensitive to an immunomodulatory therapy, via similarity to expression phenotypes displayed across the same genes by patients with the same indication and already known to be sensitive or insensitive to that therapy.

In another aspect, provided herein are methods for predicting the clinical sensitivity of a hematological cancer to treatment with an immunomodulatory therapy comprising: (a) obtaining a first biological sample from a first patient having a hematological cancer, (b) measuring the expression of a certain subset of genes set forth in Table 3 in the first biological sample, and (c) comparing the gene expression profile of the subset of genes in the first biological sample to (i) the gene expression profile of the subset of genes in tumor samples from patients having the same type of hematological cancer which are clinically sensitive to an immunomodulatory therapy and (ii) the gene expression of the subset of genes in tumor samples from patients having the same type of hematological cancer which are clinically insensitive to the immunomodulatory therapy, wherein a gene expression profile for the subset of genes in the first biological sample similar to the gene expression profile for the subset of genes in tumor samples from patients having the same type of hematological cancer which are clinically sensitive to the immunomodulatory therapy indicates that the hematological cancer in the first patient will be clinical sensitive to treatment with the immunomodulatory therapy, and a gene expression profile for the subset of genes in first biological sample similar to the gene expression profile for the subset of genes in tumor samples from patients having the same type of hematological cancer which are clinically insensitive to the immunomodulatory therapy indicates that the hematological cancer of the first patient will be clinically insensitive to the treatment with the immunomodulatory therapy. In certain embodiments, the subset of genes comprises 3, 4, 5, 6, 7, 8, 9, 10, 11, 12, 14, 15 or more of the genes in Table 3. In some embodiments, the subset of genes comprises 2-5, 5-10, 10-15, 15-20, 20-25 or 25-30 of the genes in Table 3.

In another aspect, provided herein are methods for predicting the clinical sensitivity of a hematological cancer to treatment with an immunomodulatory therapy comprising: (a) obtaining a first biological sample from a first patient having a hematological cancer, (b) measuring the expression of a certain subset of genes set forth in Table 3 in the first biological sample, and (c) comparing the gene expression profile of the subset of genes in the first biological sample to (i) the gene expression profile of the subset of genes in tumor samples from patients having the same type of hematological cancer which are clinically sensitive to an immunomodulatory therapy and (ii) the gene expression of the subset of genes in tumor samples from patients having the same type of hematological cancer which are clinically insensitive to the immunomodulatory therapy, wherein a gene expression profile for the subset of genes in the first biological sample similar to the gene expression profile for the subset of genes in tumor samples from patients having the same type of hematological cancer which are clinically sensitive to the immunomodulatory therapy indicates that the hematological cancer in the first patient will be clinical sensitive to treatment with the immunomodulatory therapy. In certain embodiments, the subset of genes comprises 3, 4, 5, 6, 7, 8, 9, 10, 11, 12, 14, 15 or more of the genes in Table 3. In some embodiments, the subset of genes comprises 2-5, 5-10, 10-15, 15-20, 20-25 or 25-30 of the genes in Table 3.

In another aspect, provided herein are methods for predicting the clinical sensitivity of a hematological cancer to treatment with an immunomodulatory therapy comprising: (a) obtaining a first biological sample from a first patient having a hematological cancer, (b) measuring the expression of a certain subset of genes set forth in Table 3 in the first biological sample, and (c) comparing the gene expression profile of the subset of genes in the first biological sample to (i) the gene expression profile of the subset of genes in tumor samples from patients having the same type of hematological cancer which are clinically sensitive to an immunomodulatory therapy and (ii) the gene expression of the subset of genes in tumor samples from patients having the same type of hematological cancer which are clinically insensitive to the immunomodulatory therapy, wherein a gene expression profile for the subset of genes in first biological sample similar to the gene expression profile for the subset of genes in tumor samples from patients having the same type of hematological cancer which are clinically insensitive to the immunomodulatory therapy indicates that the hematological cancer of the first patient will be clinically insensitive to the treatment with the immunomodulatory therapy. In certain embodiments, the subset of genes comprises 3, 4, 5, 6, 7, 8, 9, 10, 11, 12, 14, 15 or more of the genes in Table 3. In some embodiments, the subset of genes comprises 2-5, 5-10, 10-15, 15-20, 20-25 or 25-30 of the genes in Table 3.

In another aspect, provided herein are methods for predicting the clinical sensitivity of a hematological cancer to treatment with an immunomodulatory therapy comprising: (a) obtaining a first biological sample from a first patient having a hematological cancer, (b) measuring the expression of the genes or a certain subset of genes set forth in Table 4 in the first biological sample, and (c) comparing the gene expression profile of the genes or subset of genes in the first biological sample to (i) the gene expression profile of the genes or subset of genes in tumor samples from patients having the same type of hematological cancer which are clinically sensitive to an immunomodulatory therapy and (ii) the gene expression of the genes or subset of genes in Table 4 in tumor samples from patients having the same type of hematological cancer which are clinically insensitive to the immunomodulatory therapy, wherein a gene expression profile for the genes or subset of genes in the first biological sample similar to the gene expression profile for the genes or subset of genes in tumor samples from patients having the same type of hematological cancer which are clinically sensitive to the immunomodulatory therapy indicates that the hematological cancer in the first patient will be clinical sensitive to treatment with the immunomodulatory therapy, and a gene expression profile for the subset of genes in first biological sample similar to the gene expression profile for the subset of genes in tumor samples from patients having the same type of hematological cancer which are clinically insensitive to the immunomodulatory therapy indicates that the hematological cancer of the first patient will be clinically insensitive to the treatment with the immunomodulatory therapy. In certain embodiments, the subset of genes comprises 3, 4, 5, 6, 7, 8, 9, 10, 11, 12, 14, 15 or more of the genes in Table 4. In some embodiments, the subset of genes comprises 2-5, 5-10, or 10-15 of the genes in Table 4.

In another aspect, provided herein are methods for predicting the clinical sensitivity of a hematological cancer to treatment with an immunomodulatory therapy comprising: (a) obtaining a first biological sample from a first patient having a hematological cancer, (b) measuring the expression of the genes or a certain subset of genes set forth in Table 4 in the first biological sample, and (c) comparing the gene expression profile of the genes or subset of genes in the first biological sample to (i) the gene expression profile of the genes or subset of genes in tumor samples from patients having the same type of hematological cancer which are clinically sensitive to an immunomodulatory therapy and (ii) the gene expression of the genes or subset of genes in Table 4 in tumor samples from patients having the same type of hematological cancer which are clinically insensitive to the immunomodulatory therapy, wherein a gene expression profile for the genes or subset of genes in the first biological sample similar to the gene expression profile for the genes or subset of genes in tumor samples from patients having the same type of hematological cancer which are clinically sensitive to the immunomodulatory therapy indicates that the hematological cancer in the first patient will be clinical sensitive to treatment with the immunomodulatory therapy. In certain embodiments, the subset of genes comprises 3, 4, 5, 6, 7, 8, 9, 10, 11, 12, 14, 15 or more of the genes in Table 4. In some embodiments, the subset of genes comprises 2-5, 5-10, or 10-15 of the genes in Table 4.

In another aspect, provided herein are methods for predicting the clinical sensitivity of a hematological cancer to treatment with an immunomodulatory therapy comprising: (a) obtaining a first biological sample from a first patient having a hematological cancer, (b) measuring the expression of the genes or a certain subset of genes set forth in Table 4 in the first biological sample, and (c) comparing the gene expression profile of the genes or subset of genes in the first biological sample to (i) the gene expression profile of the genes or subset of genes in tumor samples from patients having the same type of hematological cancer which are clinically sensitive to an immunomodulatory therapy and (ii) the gene expression of the genes or subset of genes in Table 4 in tumor samples from patients having the same type of hematological cancer which are clinically insensitive to the immunomodulatory therapy, wherein a gene expression profile for the subset of genes in first biological sample similar to the gene expression profile for the subset of genes in tumor samples from patients having the same type of hematological cancer which are clinically insensitive to the immunomodulatory therapy indicates that the hematological cancer of the first patient will be clinically insensitive to the treatment with the immunomodulatory therapy. In certain embodiments, the subset of genes comprises 3, 4, 5, 6, 7, 8, 9, 10, 11, 12, 14, 15 or more of the genes in Table 4. In some embodiments, the subset of genes comprises 2-5, 5-10, or 10-15 of the genes in Table 4.

In another aspect, provided herein are methods for predicting the clinical sensitivity of a hematological cancer to treatment with an immunomodulatory therapy comprising: (a) obtaining a first biological sample from a first patient having a hematological cancer, (b) measuring the expression of the genes or a certain subset of genes set forth in Table 1 in the first biological sample, and (c) comparing the gene expression profile of the genes or subset of genes in the first biological sample to (i) the gene expression profile of the genes or subset of genes in tumor samples from patients having the same type of hematological cancer which are clinically sensitive to an immunomodulatory therapy and (ii) the gene expression of the genes or subset of genes in tumor samples from patients having the same type of hematological cancer which are clinically insensitive to the immunomodulatory therapy, wherein a gene expression profile for the genes or subset of genes in the first biological sample similar to the gene expression profile for the subset of genes in tumor samples from patients having the same type of hematological cancer which are clinically sensitive to the immunomodulatory therapy indicates that the hematological cancer in the first patient will be clinical sensitive to treatment with the immunomodulatory therapy, and a gene expression profile for the genes or subset of genes in first biological sample similar to the gene expression profile for the genes or subset of genes in tumor samples from patients having the same type of hematological cancer which are clinically insensitive to the immunomodulatory therapy indicates that the hematological cancer of the first patient will be clinically insensitive to the treatment with the immunomodulatory therapy. In certain embodiments, the subset of genes comprises 3, 4, 5, 6, 7, 8, 9, 10, 11, 12, 14, 15 or more of the genes in Table 1. In some embodiments, the subset of genes comprises 2-5, 5-10, 10-15, 15-20, 20-25 or 25-30 of the genes in Table 1.

In another aspect, provided herein are methods for predicting the clinical sensitivity of a hematological cancer to treatment with an immunomodulatory therapy comprising: (a) obtaining a first biological sample from a first patient having a hematological cancer, (b) measuring the expression of the genes or a certain subset of genes set forth in Table 1 in the first biological sample, and (c) comparing the gene expression profile of the genes or subset of genes in the first biological sample to (i) the gene expression profile of the genes or subset of genes in tumor samples from patients having the same type of hematological cancer which are clinically sensitive to an immunomodulatory therapy and (ii) the gene expression of the genes or subset of genes in tumor samples from patients having the same type of hematological cancer which are clinically insensitive to the immunomodulatory therapy, wherein a gene expression profile for the genes or subset of genes in the first biological sample similar to the gene expression profile for the subset of genes in tumor samples from patients having the same type of hematological cancer which are clinically sensitive to the immunomodulatory therapy indicates that the hematological cancer in the first patient will be clinical sensitive to treatment with the immunomodulatory therapy. In certain embodiments, the subset of genes comprises 3, 4, 5, 6, 7, 8, 9, 10, 11, 12, 14, 15 or more of the genes in Table 1. In some embodiments, the subset of genes comprises 2-5, 5-10, 10-15, 15-20, 20-25 or 25-30 of the genes in Table 1.

In another aspect, provided herein are methods for predicting the clinical sensitivity of a hematological cancer to treatment with an immunomodulatory therapy comprising: (a) obtaining a first biological sample from a first patient having a hematological cancer, (b) measuring the expression of the genes or a certain subset of genes set forth in Table 1 in the first biological sample, and (c) comparing the gene expression profile of the genes or subset of genes in the first biological sample to (i) the gene expression profile of the genes or subset of genes in tumor samples from patients having the same type of hematological cancer which are clinically sensitive to an immunomodulatory therapy and (ii) the gene expression of the genes or subset of genes in tumor samples from patients having the same type of hematological cancer which are clinically insensitive to the immunomodulatory therapy, wherein a gene expression profile for the genes or subset of genes in first biological sample similar to the gene expression profile for the genes or subset of genes in tumor samples from patients having the same type of hematological cancer which are clinically insensitive to the immunomodulatory therapy indicates that the hematological cancer of the first patient will be clinically insensitive to the treatment with the immunomodulatory therapy. In certain embodiments, the subset of genes comprises 3, 4, 5, 6, 7, 8, 9, 10, 11, 12, 14, 15 or more of the genes in Table 1. In some embodiments, the subset of genes comprises 2-5, 5-10, 10-15, 15-20, 20-25 or 25-30 of the genes in Table 1.

In another aspect, provided herein are methods for predicting the clinical sensitivity of a hematological cancer to treatment with an immunomodulatory therapy comprising: (a) obtaining a first biological sample from a first patient having a hematological cancer, (b) measuring the expression of the genes or a certain subset of genes set forth in Table 2 in the first biological sample, and (c) comparing the gene expression profile of the genes or subset of genes in the first biological sample to (i) the gene expression profile of the genes or subset of genes in tumor samples from patients having the same type of hematological cancer which are clinically sensitive to an immunomodulatory therapy and (ii) the gene expression of the genes or subset of genes in tumor samples from patients having the same type of hematological cancer which are clinically insensitive to the immunomodulatory therapy, wherein a gene expression profile for the genes or subset of genes in the first biological sample similar to the gene expression profile for the genes or subset of genes in tumor samples from patients having the same type of hematological cancer which are clinically sensitive to the immunomodulatory therapy indicates that the hematological cancer in the first patient will be clinical sensitive to treatment with the immunomodulatory therapy, and a gene expression profile for the genes or subset of genes in first biological sample similar to the gene expression profile for the genes or subset of genes in tumor samples from patients having the same type of hematological cancer which are clinically insensitive to the immunomodulatory therapy indicates that the hematological cancer of the first patient will be clinically insensitive to the treatment with the immunomodulatory therapy. In certain embodiments, the subset of genes comprises 3, 4, 5, 6, 7, 8, 9, 10, 11, 12, 14, 15 or more of the genes in Table 2. In some embodiments, the subset of genes comprises 2-5, 5-10, 10-15, 15-20, 20-25 or 25-30 of the genes in Table 2.

In another aspect, provided herein are methods for predicting the clinical sensitivity of a hematological cancer to treatment with an immunomodulatory therapy comprising: (a) obtaining a first biological sample from a first patient having a hematological cancer, (b) measuring the expression of the genes or a certain subset of genes set forth in Table 2 in the first biological sample, and (c) comparing the gene expression profile of the genes or subset of genes in the first biological sample to (i) the gene expression profile of the genes or subset of genes in tumor samples from patients having the same type of hematological cancer which are clinically sensitive to an immunomodulatory therapy and (ii) the gene expression of the genes or subset of genes in tumor samples from patients having the same type of hematological cancer which are clinically insensitive to the immunomodulatory therapy, wherein a gene expression profile for the genes or subset of genes in the first biological sample similar to the gene expression profile for the genes or subset of genes in tumor samples from patients having the same type of hematological cancer which are clinically sensitive to the immunomodulatory therapy indicates that the hematological cancer in the first patient will be clinical sensitive to treatment with the immunomodulatory therapy. In certain embodiments, the subset of genes comprises 3, 4, 5, 6, 7, 8, 9, 10, 11, 12, 14, 15 or more of the genes in Table 2. In some embodiments, the subset of genes comprises 2-5, 5-10, 10-15, 15-20, 20-25 or 25-30 of the genes in Table 2.

In another aspect, provided herein are methods for predicting the clinical sensitivity of a hematological cancer to treatment with an immunomodulatory therapy comprising: (a) obtaining a first biological sample from a first patient having a hematological cancer, (b) measuring the expression of the genes or a certain subset of genes set forth in Table 2 in the first biological sample, and (c) comparing the gene expression profile of the genes or subset of genes in the first biological sample to (i) the gene expression profile of the genes or subset of genes in tumor samples from patients having the same type of hematological cancer which are clinically sensitive to an immunomodulatory therapy and (ii) the gene expression of the genes or subset of genes in tumor samples from patients having the same type of hematological cancer which are clinically insensitive to the immunomodulatory therapy, wherein a gene expression profile for the genes or subset of genes in first biological sample similar to the gene expression profile for the genes or subset of genes in tumor samples from patients having the same type of hematological cancer which are clinically insensitive to the immunomodulatory therapy indicates that the hematological cancer of the first patient will be clinically insensitive to the treatment with the immunomodulatory therapy. In certain embodiments, the subset of genes comprises 3, 4, 5, 6, 7, 8, 9, 10, 1, 12, 14, 15 or more of the genes in Table 2. In some embodiments, the subset of genes comprises 2-5, 5-10, 10-15, 15-20, 20-25 or 25-30 of the genes in Table 2.

In accordance with the methods described herein, the biological sample can be any sample obtained from the patient. In certain embodiments, the biological sample is a cell sample. In other embodiments, the biological sample is whole blood sample, peripheral blood mononuclear cell sample, or tissue sample. In specific embodiments, the biological sample is a tumor sample. See Section 5.8, infra, regarding biological samples.

In accordance with the methods described herein, the hematological cancer can be any hematological cancer. Examples of hematological cancers can be found in Section 5.5, infra. In a specific embodiment, the hematological cancer is a lymphoma. In another specific embodiment, the hematological cancer is a non-Hodgkin's lymphoma. In yet another embodiment, the hematological cancer is a diffuse large B-cell lymphoma (DLBCL). In certain embodiments, the DLBCL is a germinal center B-cell-like DLBCL. In other embodiments, the DLBCL is an activated B-cell-like DLBCL.

In accordance with the methods described herein, the level of expression of one, two, three, four, five or more of the genes in Table 1, Table 2, Table 3, and/or Table 4, infra, can be measured at the RNA and/or protein levels. In certain embodiments, the level of expression of the genes are measured at the RNA (e.g., mRNA) level. In other embodiments, the level of expression of the genes are measured at the protein level.

Techniques known to one skilled in the art may be used to measure the amount of an RNA transcript(s). In some embodiments, the amount of one, two, three, four, five or more RNA transcripts is measured using deep sequencing, such as ILLUMINA® RNASeq, ILLUMINA® next generation sequencing (NGS), ION TORRENT™ RNA next generation sequencing, 454™ pyrosequencing, or Sequencing by Oligo Ligation Detection (SOLID™). In other embodiments, the amount of multiple RNA transcripts is measured using a microarray and/or gene chip, such as described in Section 6, infra. In certain embodiments, the amount of one, two, three or more RNA transcripts is determined by RT-PCR. In other embodiments, the amount of one, two, three or more RNA transcripts is measured by RT-qPCR. Techniques for conducting these assays are known to one skilled in the art. See Section 5.9, infra, for examples of assays to measure RNA transcripts.

In some embodiments, a statistical analysis or other analysis is performed on data from the assay utilized to measure an RNA transcript or protein. In certain specific embodiments, p value of those RNA transcripts or proteins differentially expressed is 0.1, 0.5, 0.4, 0.3, 0.2, 0.01, 0.05, 0.001, 0.005, or 0.0001. In specific embodiments, a false discovery rate (FDR) of 10%, 9%, 8%, 7%, 6%, 5%, 4%, 3%, 2%, 1% or less is selected.

Techniques known to one skilled in the art may be used to measure the amount of a protein. For example, flow cytometry, immunofluorescence, enzyme-linked immunosorbent assay-based methodologies (ELISA) and similar assays known in the art. See Section 5.10, infra, for examples of assays to measure protein.

In accordance with the methods described herein, the immunomodulatory therapy can be any therapy that modulates the immune system or immune response. Examples of immunomodulatory therapies are provided in Section 5.6, infra. In a specific embodiment, the immunomodulatory therapy is lenalidomide (Revlimid®).

### 5.3 Methods for Predicting Clinical Sensitivity of a Hematological Cancer by Measuring Proportion of Cells

In another aspect, provided herein are methods for predicting the clinical sensitivity of a hematological cancer to treatment with an immunomodulatory therapy comprising: (a) obtaining a first tumor sample from a first patient having the hematological cancer, (b) measuring the proportion of dendritic cells in the first tumor sample, and (c) comparing the proportion of dendritic cells in the first tumor sample with the proportion of dendritic cells in a second tumor sample from a second patient having the same type of hematological cancer, wherein the second patient's hematological cancer is clinically insensitive to treatment with the immunomodulatory therapy, and wherein a higher proportion of dendritic cells in the first tumor sample relative the proportion of dendritic cells in the second tumor sample indicates that the hematological cancer in the first patient will be clinical sensitive to treatment with the immunomodulatory therapy.

In another aspect, provided herein are methods for predicting the clinical sensitivity of a hematological cancer to treatment with an immunomodulatory therapy comprising: (a) obtaining a first tumor sample from a first patient having the hematological cancer, (b) measuring the proportion of plasma cells in the first tumor sample, and (c) comparing the proportion of plasma cells in the first tumor sample with the proportion of plasma cells in a second tumor sample from a second patient having the same type of hematological cancer, wherein the second patient's hematological cancer is clinically insensitive to treatment with the immunomodulatory therapy, and wherein a higher proportion of plasma cells in the first tumor sample relative the proportion of plasma cells in the second tumor sample indicates that the hematological cancer in the first patient will be clinical sensitive to treatment with the immunomodulatory therapy.

In another aspect, provided herein are methods for predicting the clinical sensitivity of a hematological cancer to treatment with an immunomodulatory therapy comprising: (a) obtaining a first tumor sample from a first patient having the hematological cancer, (b) measuring the proportion of dendritic cells and plasma cells in the first tumor sample, and (c) comparing the proportion of dendritic cells and plasma cells in the first tumor sample with the proportion of dendritic cells and plasma cells in a second tumor sample from a second patient having the same type of hematological cancer, wherein the second patient's hematological cancer is clinically insensitive to treatment with the immunomodulatory therapy, and wherein a higher proportion of dendritic cells and plasma cells in the first tumor sample relative the proportion of dendritic cells and plasma cells in the second tumor sample indicates that the hematological cancer in the first patient will be clinical sensitive to treatment with the immunomodulatory therapy.

In another aspect, provided herein are methods for predicting the clinical sensitivity of a hematological cancer to treatment with an immunomodulatory therapy comprising: (a) obtaining a first tumor sample from a first patient having the hematological cancer, (b) measuring the proportion of B cells in the first tumor sample, and (c) comparing the proportion of B cells in the first tumor sample with the proportion of B cells in a second tumor sample from a second patient having the same type of hematological cancer, wherein the second patient's hematological cancer is clinically insensitive to treatment with the immunomodulatory therapy, and wherein a decreased proportion of B cells in the first tumor sample relative the proportion of B cells in the second tumor sample indicates that the hematological cancer in the first patient will be clinical sensitive to treatment with the immunomodulatory therapy.

In another aspect, provided herein are methods for predicting the clinical sensitivity of a hematological cancer to treatment with an immunomodulatory therapy comprising: (a) obtaining a first tumor sample from a first patient having the hematological cancer, (b) measuring the proportion of Natural Killer (NK) cells in the first tumor sample, and (c) comparing the proportion of NK cells in the first tumor sample with the proportion of NK cells in a second tumor sample from a second patient having the same type of hematological cancer, wherein the second patient's hematological cancer is clinically insensitive to treatment with the immunomodulatory therapy, and wherein a higher proportion of NK cells in the first tumor sample relative the proportion of NK cells in the second tumor sample indicates that the hematological cancer in the first patient will be clinical sensitive to treatment with the immunomodulatory therapy.

In another aspect, provided herein are methods for predicting the clinical sensitivity of a hematological cancer to treatment with an immunomodulatory therapy comprising: (a) obtaining a first tumor sample from a first patient having the hematological cancer, (b) measuring the proportion of tumor infiltrating immune cells in the first tumor sample, and (c) comparing the proportion of tumor infiltrating immune cells in the first tumor sample with the proportion of tumor infiltrating immune cells in a second tumor sample from a second patient having the same type of hematological cancer, wherein the second patient's hematological cancer is clinically insensitive to treatment with the immunomodulatory therapy, and wherein a higher proportion of tumor infiltrating immune cells in the first tumor sample relative the proportion of tumor infiltrating immune cells in the second tumor sample indicates that the hematological cancer in the first patient will be clinical sensitive to treatment with the immunomodulatory therapy.

In another aspect, provided herein are methods for predicting the clinical sensitivity of a hematological cancer to treatment with an immunomodulatory therapy comprising: (a) obtaining a first tumor sample from a first patient having the hematological cancer, (b) measuring the proportion of monocytes in the first tumor sample, and (c) comparing the proportion of NK cells in the first tumor sample with the proportion of monocytes in a second tumor sample from a second patient having the same type of hematological cancer, wherein the second patient's hematological cancer is clinically insensitive to treatment with the immunomodulatory therapy, and wherein a higher proportion of monocytes in the first tumor sample relative the proportion of monocytes in the second tumor sample indicates that the hematological cancer in the first patient will be clinical sensitive to treatment with the immunomodulatory therapy.

In certain embodiments of the foregoing paragraphs in this section, the second patient is a single patient. In other embodiments of the foregoing paragraphs in this section, the second patient is a population of patients. In specific embodiments, the population comprises 5, 10, 15, 20, 25, 30, 35, 40, 45, 50, 55, 60, 65, 70, 75, 80, 85, 90, 95, 100, 125, 150, 200, 225, 250, 300 or more patients.

In another aspect, provided herein are methods for predicting the clinical sensitivity of a hematological cancer to treatment with an immunomodulatory therapy comprising: (a) obtaining a first tumor sample from a first patient having a hematological cancer, (b) measuring the proportion of immune cells in the tumor sample, and (c) comparing the proportion of the immune cells in the first tumor sample to (i) the proportion of the same immune cells in tumor samples from patients having the same type of hematological cancer which are clinically sensitive to an immunomodulatory therapy and (ii) the proportion of the same immune cells in tumor samples from patients having the same type of hematological cancer which are clinically insensitive to the immunomodulatory therapy, wherein a proportion of the immune cells in the first tumor sample similar to the proportion of the same immune cells in tumor samples from patients having the same type of hematological cancer which are clinically sensitive to the immunomodulatory therapy indicates that the hematological cancer in the first patient will be clinical sensitive to treatment with the immunomodulatory therapy. In some embodiments, the immune cells are subset of immune cells, such as subset of B cells. In certain embodiments, the immune cells are dendritic cells. In some embodiments, the immune cells are plasma cells. In certain embodiments, the immune cells are monocytes. In some embodiments, the immune cells are tumor infiltrating immune cells. In certain embodiments, the immune cells are T cells. In some embodiments, the immune cells are B cells. In certain embodiments, the immune cells are NK cells. In some embodiments, the immune cells are two, three or more subsets of immune cells, such as two more types of T cells (e.g., CD4+ and CD8+ T cells). In some embodiments, the proportion of different populations of immune cells in the first tumor sample are compared to (i) the proportion of the same populations of immune cells in the tumor samples from patients having the same type of hematological cancer which are clinically sensitive to the immunomodulatory therapy and (ii) the proportion of the same populations of immune cells in the tumor samples from patients having the same type of hematological cancer which are clinically insensitive to the immunomodulatory therapy.

In another aspect, provided herein are methods for predicting the clinical sensitivity of a hematological cancer to treatment with an immunomodulatory therapy comprising: (a) obtaining a first tumor sample from a first patient having a hematological cancer, (b) measuring the proportion of immune cells in the tumor sample, and (c) comparing the proportion of the immune cells in the first tumor sample to (i) the proportion of the same immune cells in tumor samples from patients having the same type of hematological cancer which are clinically sensitive to an immunomodulatory therapy and (ii) the proportion of the same immune cells in tumor samples from patients having the same type of hematological cancer which are clinically insensitive to the immunomodulatory therapy, wherein a proportion of the immune cells in the first tumor sample similar to the proportion of the same immune cells in tumor samples from patients having the same type of hematological cancer which are clinically insensitive to the immunomodulatory therapy indicates that the hematological cancer of the first patient will be clinically insensitive to the treatment with the immunomodulatory therapy. In some embodiments, the immune cells are subset of immune cells, such as subset of B cells. In certain embodiments, the immune cells are dendritic cells. In some embodiments, the immune cells are plasma cells. In certain embodiments, the immune cells are monocytes. In some embodiments, the immune cells are tumor infiltrating immune cells. In certain embodiments, the immune cells are T cells. In some embodiments, the immune cells are B cells. In certain embodiments, the immune cells are NK cells. In some embodiments, the immune cells are two, three or more subsets of immune cells, such as two more types of T cells (e.g., CD4+ and CD8+ T cells). In some embodiments, the proportion of different populations of immune cells in the first tumor sample are compared to (i) the proportion of the same populations of immune cells in the tumor samples from patients having the same type of hematological cancer which are clinically sensitive to the immunomodulatory therapy and (ii) the proportion of the same populations of immune cells in the tumor samples from patients having the same type of hematological cancer which are clinically insensitive to the immunomodulatory therapy.

In another aspect, provided herein are methods for predicting the clinical sensitivity of a hematological cancer to treatment with an immunomodulatory therapy comprising: (a) obtaining a first tumor sample from a first patient having a hematological cancer, (b) measuring the proportion of immune cells in the tumor sample, and (c) comparing the proportion of the immune cells in the first tumor sample to (i) the proportion of the same immune cells in tumor samples from patients having the same type of hematological cancer which are clinically sensitive to an immunomodulatory therapy and (ii) the proportion of the same immune cells in tumor samples from patients having the same type of hematological cancer which are clinically insensitive to the immunomodulatory therapy, wherein a proportion of the immune cells in the first tumor sample similar to the proportion of the same immune cells in tumor samples from patients having the same type of hematological cancer which are clinically sensitive to the immunomodulatory therapy indicates that the hematological cancer in the first patient, and a proportion of the immune cells in the first tumor sample similar to the proportion of the same immune cells in tumor samples from patients having the same type of hematological cancer which are clinically insensitive to the immunomodulatory therapy indicates that the hematological cancer of the first patient will be clinically insensitive to the treatment with the immunomodulatory therapy. In some embodiments, the immune cells are subset of immune cells, such as subset of B cells. In certain embodiments, the immune cells are dendritic cells. In some embodiments, the immune cells are plasma cells. In certain embodiments, the immune cells are monocytes. In some embodiments, the immune cells are tumor infiltrating immune cells. In certain embodiments, the immune cells are T cells. In some embodiments, the immune cells are B cells. In certain embodiments, the immune cells are NK cells. In some embodiments, the immune cells are two, three or more subsets of immune cells, such as two more types of T cells (e.g., CD4+ and CD8+ T cells). In some embodiments, the proportion of different populations of immune cells in the first tumor sample are compared to (i) the proportion of the same populations of immune cells in the tumor samples from patients having the same type of hematological cancer which are clinically sensitive to the immunomodulatory therapy and (ii) the proportion of the same populations of immune cells in the tumor samples from patients having the same type of hematological cancer which are clinically insensitive to the immunomodulatory therapy.

In addition to measuring the cells (e.g., dendritic cells, plasma cells, B cells, monocytes, and infiltrating immune cells) in a tumor sample from a patient, levels of expression of genes (e.g., one, two, three, four, five or more of the genes in Table 1 and/or Table 2) may be assessed. In specific embodiments, the methods set forth in Section 5.1, supra, are combined with the methods set forth in this Section 5.2 to predict the clinical sensitivity of a hematological cancer to treatment with an immunomodulatory therapy.

Techniques known to one skilled in the art may be used to measure the proportion of cells in a tumor sample. In certain embodiments, the proportion of cells is measured by flow cytometry, immunofluorescence, enzyme-linked immunosorbent assay-based methodologies (ELISA) and similar assays known in the art. See Section 5.8, infra, regarding techniques for measuring and distinguishing cell types. In other embodiments, the proportion of cells is measured by inference from gene expression profiles.

In accordance with the methods described herein, the hematological cancer can be any hematological cancer. Examples of hematological cancers can be found in Section 5.5, infra. In a specific embodiment, the hematological cancer is a lymphoma. In another specific embodiment, the hematological cancer is a non-Hodgkin's lymphoma. In yet another embodiment, the hematological cancer is a diffuse large B-cell lymphoma (DLBCL). In certain embodiments, the DLBCL is a germinal center B-cell-like DLBCL. In other embodiments, the DLBCL is an activated B-cell-like DLBCL.

In accordance with the methods described herein, the immunomodulatory therapy can be any therapy that modulates the immune system or immune response. Examples of immunomodulatory therapies are provided in Section 5.6, infra. In a specific embodiment, the immunomodulatory therapy is lenalidomide (Revlimid®).

### 5.4 Methods for Managing and Treating Hematological Cancer

In one aspect, provided herein are methods for managing or treating a hematological cancer comprising: (a) obtaining a first biological sample from a first patient having a hematological cancer, (b) measuring the level of expression of one, two, three, four, five or more of the genes identified in Table 3 or 4, infra, (c) comparing the level of expression of the one, two, three, four, five or more of the genes identified in Table 3 or 4 in the first biological sample with the level of expression of the same genes in a second biological sample from a second patient having the same type of hematological cancer as the first patient, wherein the hematological cancer in the second patient is clinically insensitive to an immunomodulatory therapy, and (d) administering the immunomodulatory therapy to the first patient if the one, two, three, four, five or more of the genes in the first biological sample are differentially expressed relative to the level of expression of the one, two, three, four, five or more of the genes in the second biological sample. In certain embodiments, the immunomodulatory therapy is not administered or additional assays are conducted if the level of expression of one, two, three, four, five or more of the genes are not higher in the first biological sample than in the second biological sample.

In another aspect, provided herein are methods for managing or treating a hematological cancer comprising: (a) obtaining a first biological sample from a first patient having a hematological cancer, (b) measuring the level of expression of one, two, three, four, five or more of the genes identified in Table 1, infra, (c) comparing the level of expression of the one, two, three, four, five or more of the genes identified in Table 1 in the first biological sample with the level of expression of the same genes in a second biological sample from a second patient having the same type of hematological cancer as the first patient, wherein the hematological cancer in the second patient is clinically insensitive to an immunomodulatory therapy, and (d) administering the immunomodulatory therapy to the first patient if a higher level of expression of the one, two, three, four, five or more of the genes in the first biological sample is measured relative to the level of expression of the one, two, three, four, five or more of the genes in the second biological sample. In certain embodiments, the immunomodulatory therapy is not administered or additional assays are conducted if the level of expression of one, two, three, four, five or more of the genes are not higher in the first biological sample than in the second biological sample.

In another aspect, provided herein are methods for managing or treating a hematological cancer comprising: (a) obtaining a first biological sample from a first patient having a hematological cancer, (b) measuring the level of expression of one, two, three, four, five or more of the genes identified in Table 2, infra, (c) comparing the level of expression of the one, two, three, four, five or more of the genes identified in Table 2 in the first biological sample with the level of expression of the same genes in a second biological sample is from a second patient having the same type of hematological cancer as the first patient, wherein the hematological cancer in the second patient is clinically insensitive to an immunomodulatory therapy, and (d) administering the immunomodulatory therapy to the first patient if a lower level of expression of the one, two, three, four, five or more of the genes in the first biological sample is measured relative to the level of expression of the one, two, three, four, five or more of the genes in the second biological sample. In certain embodiments, the immunomodulatory therapy is not administered or additional assays are conducted if the level of expression of one, two, three, four, five or more of the genes are not lower in the first biological sample than in the second biological sample. In certain embodiments, the immunomodulatory therapy is not administered or additional assays are conducted if the level of expression of one, two, three, four, five or more of the genes are not higher in the first biological sample than in the second biological sample.

In another aspect, provided herein are methods for managing or treating a hematological cancer comprising: (a) obtaining a first biological sample from a first patient having a hematological cancer, (b) measuring the level of expression of one, two, three, four, five or more of the genes identified in Table 1, infra, and measuring the level of expression one, two, three, four, five or more of the genes identified in Table 2, infra, (c) comparing the level of expression of the genes identified in Tables 1 and 2 in the first biological sample with the level of expression of the same genes in a second biological sample is from a second patient having the same type of hematological cancer as the first patient, wherein the hematological cancer in the second patient is clinically insensitive to an immunomodulatory therapy, and (d) administering the immunomodulatory to the first patient if (i) a higher level of expression of the one, two, three, four, five or more of the genes identified in Table 1 in the first biological sample is measured relative to the level of expression of the one, two, three, four, five or more of the genes in the second biological sample, and (ii) a lower level of expression of the one, two, three, four, five or more of the genes identified in Table 2 in the first biological sample is measured relative to the level of expression of the one, two, three, four, five or more of the genes in the second biological sample. In certain embodiments, the immunomodulatory therapy is not administered or additional assays are conducted if the level of expression of one, two, three, four, five or more of the genes are not higher in the first biological sample than in the second biological sample.

In another aspect, provided herein are methods for managing or treating a hematological cancer comprising: (a) obtaining a first biological sample from a first patient having a hematological cancer, (b) measuring the expression of a certain subset of genes set forth in Table 3, infra, in the first biological sample, and (c) comparing the gene expression profile of the subset of genes in the first biological sample to (i) the gene expression profile of the subset of genes in tumor samples from patients having the same type of hematological cancer which are clinically sensitive to an immunomodulatory therapy and (ii) the gene expression of the subset of genes in tumor samples from patients having the same type of hematological cancer which are clinically insensitive to the immunomodulatory therapy, and (d) administering the immunomodulatory therapy to the first patient if the gene expression profile for the subset of genes in the first biological sample is similar to the gene expression profile for the subset of genes in tumor samples from patients having the same type of hematological cancer which are clinically sensitive to the immunomodulatory therapy. In certain embodiments, the subset of genes comprises 3, 4, 5, 6, 7, 8, 9, 10, 11, 12, 14, 15 or more of the genes in Table 3. In some embodiments, the subset of genes comprises 2-5, 5-10, 10-15, 15-20, 20-25 or 25-30 of the genes in Table 3.

In another aspect, provided herein are methods for managing or treating a hematological cancer comprising: (a) obtaining a first biological sample from a first patient having a hematological cancer, (b) measuring the expression of a certain subset of genes set forth in Table 3, infra, in the first biological sample, and (c) comparing the gene expression profile of the subset of genes in the first biological sample to (i) the gene expression profile of the subset of genes in tumor samples from patients having the same type of hematological cancer which are clinically sensitive to an immunomodulatory therapy and (ii) the gene expression of the subset of genes in tumor samples from patients having the same type of hematological cancer which are clinically insensitive to the immunomodulatory therapy, and (d) administering the immunomodulatory therapy to the first patient if the gene expression profile for the subset of genes in the first biological sample is not similar to the gene expression profile for the subset of genes in tumor samples from patients having the same type of hematological cancer which are clinically insensitive to the immunomodulatory therapy. In certain embodiments, the subset of genes comprises 3, 4, 5, 6, 7, 8, 9, 10, 11, 12, 14, 15 or more of the genes in Table 3. In some embodiments, the subset of genes comprises 2-5, 5-10, 10-15, 15-20, 20-25 or 25-30 of the genes in Table 3.

In another aspect, provided herein are methods for managing or treating a hematological cancer comprising: (a) obtaining a first biological sample from a first patient having a hematological cancer, (b) measuring the expression of a certain subset of genes set forth in Table 3, infra, in the first biological sample, and (c) comparing the gene expression profile of the subset of genes in the first biological sample to (i) the gene expression profile of the subset of genes in tumor samples from patients having the same type of hematological cancer which are clinically sensitive to an immunomodulatory therapy and (ii) the gene expression of the subset of genes in tumor samples from patients having the same type of hematological cancer which are clinically insensitive to the immunomodulatory therapy, and (d) administering the immunomodulatory therapy to the first patient if: (i) the gene expression profile for the subset of genes in the first biological sample is similar to the gene expression profile for the subset of genes in tumor samples from patients having the same type of hematological cancer which are clinically sensitive to the immunomodulatory therapy and (ii) the gene expression profile for the subset of genes in first biological sample is not similar to the gene expression profile for the subset of genes in tumor samples from patients having the same type of hematological cancer which are clinically insensitive to the immunomodulatory. In certain embodiments, the subset of genes comprises 3, 4, 5, 6, 7, 8, 9, 10, 11, 12, 14, 15 or more of the genes in Table 3. In some embodiments, the subset of genes comprises 2-5, 5-10, 10-15, 15-20, 20-25 or 25-30 of the genes in Table 3.

In another aspect, provided herein are methods for managing or treating a hematological cancer comprising: (a) obtaining a first biological sample from a first patient having a hematological cancer, (b) measuring the expression of the genes or a certain subset of genes set forth in Table 4, infra, in the first biological sample, and (c) comparing the gene expression profile of the genes or a subset of genes in the first biological sample to (i) the gene expression profile of the genes or subset of genes in tumor samples from patients having the same type of hematological cancer which are clinically sensitive to an immunomodulatory therapy and (ii) the gene expression of the genes or subset of genes in tumor samples from patients having the same type of hematological cancer which are clinically insensitive to the immunomodulatory therapy, and (d) administering the immunomodulatory therapy to the first patient if the gene expression profile for the genes or subset of genes in the first biological sample is similar to the gene expression profile for the genes or subset of genes in tumor samples from patients having the same type of hematological cancer which are clinically sensitive to the immunomodulatory therapy. In certain embodiments, the subset of genes comprises 3, 4, 5, 6, 7, 8, 9, 10, 11, 12, 14, 15 or more of the genes in Table 4. In some embodiments, the subset of genes comprises 2-5, 5-10, 10-15, 15-20, 20-25 or 25-30 of the genes in Table 4.

In another aspect, provided herein are methods for managing or treating a hematological cancer comprising: (a) obtaining a first biological sample from a first patient having a hematological cancer, (b) measuring the expression of the genes or a certain subset of genes set forth in Table 4, infra, in the first biological sample, and (c) comparing the gene expression profile of the genes or subset of genes in the first biological sample to (i) the gene expression profile of the genes or subset of genes in tumor samples from patients having the same type of hematological cancer which are clinically sensitive to an immunomodulatory therapy and (ii) the gene expression of the genes or subset of genes in tumor samples from patients having the same type of hematological cancer which are clinically insensitive to the immunomodulatory therapy, and (d) administering the immunomodulatory therapy to the first patient if the gene expression profile for the genes or subset of genes in the first biological sample is not similar to the gene expression profile for the genes or subset of genes in tumor samples from patients having the same type of hematological cancer which are clinically insensitive to the immunomodulatory therapy. In certain embodiments, the subset of genes comprises 3, 4, 5, 6, 7, 8, 9, 10, 11, 12, 14, 15 or more of the genes in Table 4. In some embodiments, the subset of genes comprises 2-5, 5-10, 10-15, 15-20, 20-25 or 25-30 of the genes in Table 4.

In another aspect, provided herein are methods for managing or treating a hematological cancer comprising: (a) obtaining a first biological sample from a first patient having a hematological cancer, (b) measuring the expression of the genes or a certain subset of genes set forth in Table 4, infra, in the first biological sample, and (c) comparing the gene expression profile of the genes or subset of genes in the first biological sample to (i) the gene expression profile of the genes or subset of genes in tumor samples from patients having the same type of hematological cancer which are clinically sensitive to an immunomodulatory therapy and (ii) the gene expression of the genes or subset of genes in tumor samples from patients having the same type of hematological cancer which are clinically insensitive to the immunomodulatory therapy, and (d) administering the immunomodulatory therapy to the first patient if: (i) the gene expression profile for the genes or subset of genes in the first biological sample is similar to the gene expression profile for the genes or subset of genes in tumor samples from patients having the same type of hematological cancer which are clinically sensitive to the immunomodulatory therapy and (ii) the gene expression profile for the genes or subset of genes in first biological sample is not similar to the gene expression profile for the genes or subset of genes in tumor samples from patients having the same type of hematological cancer which are clinically insensitive to the immunomodulatory. In certain embodiments, the subset of genes comprises 3, 4, 5, 6, 7, 8, 9, 10, 11, 12, 14, 15 or more of the genes in Table 4. In some embodiments, the subset of genes comprises 2-5, 5-10, 10-15, 15-20, 20-25 or 25-30 of the genes in Table 4.

In another aspect, provided herein are methods for managing or treating a hematological cancer comprising: (a) obtaining a first biological sample from a first patient having a hematological cancer, (b) measuring the expression of the genes or a certain subset of genes set forth in Table 1, infra, in the first biological sample, and (c) comparing the gene expression profile of the genes or a subset of genes in the first biological sample to (i) the gene expression profile of the genes or subset of genes in tumor samples from patients having the same type of hematological cancer which are clinically sensitive to an immunomodulatory therapy and (ii) the gene expression of the genes or subset of genes in tumor samples from patients having the same type of hematological cancer which are clinically insensitive to the immunomodulatory therapy, and (d) administering the immunomodulatory therapy to the first patient if the gene expression profile for the genes or subset of genes in the first biological sample is similar to the gene expression profile for the genes or subset of genes in tumor samples from patients having the same type of hematological cancer which are clinically sensitive to the immunomodulatory therapy. In certain embodiments, the subset of genes comprises 3, 4, 5, 6, 7, 8, 9, 10, 11, 12, 14, 15 or more of the genes in Table 1. In some embodiments, the subset of genes comprises 2-5, 5-10, 10-15, 15-20, 20-25 or 25-30 of the genes in Table 1.

In another aspect, provided herein are methods for managing or treating a hematological cancer comprising: (a) obtaining a first biological sample from a first patient having a hematological cancer, (b) measuring the expression of the genes or a certain subset of genes set forth in Table 1, infra, in the first biological sample, and (c) comparing the gene expression profile of the genes or subset of genes in the first biological sample to (i) the gene expression profile of the genes or subset of genes in tumor samples from patients having the same type of hematological cancer which are clinically sensitive to an immunomodulatory therapy and (ii) the gene expression of the genes or subset of genes i in tumor samples from patients having the same type of hematological cancer which are clinically insensitive to the immunomodulatory therapy, and (d) administering the immunomodulatory therapy to the first patient if the gene expression profile for the genes or subset of genes in the first biological sample is not similar to the gene expression profile for the genes or subset of genes in tumor samples from patients having the same type of hematological cancer which are clinically insensitive to the immunomodulatory therapy. In certain embodiments, the subset of genes comprises 3, 4, 5, 6, 7, 8, 9, 10, 11, 12, 14, 15 or more of the genes in Table 1. In some embodiments, the subset of genes comprises 2-5, 5-10, 10-15, 15-20, 20-25 or 25-30 of the genes in Table 1.

In another aspect, provided herein are methods for managing or treating a hematological cancer comprising: (a) obtaining a first biological sample from a first patient having a hematological cancer, (b) measuring the expression of the genes or a certain subset of genes set forth in Table 1, infra, in the first biological sample, and (c) comparing the gene expression profile of the genes or subset of genes in the first biological sample to (i) the gene expression profile of the genes or subset of genes in tumor samples from patients having the same type of hematological cancer which are clinically sensitive to an immunomodulatory therapy and (ii) the gene expression of the genes or subset of genes in tumor samples from patients having the same type of hematological cancer which are clinically insensitive to the immunomodulatory therapy, and (d) administering the immunomodulatory therapy to the first patient if: (i) the gene expression profile for the genes or subset of genes in the first biological sample is similar to the gene expression profile for the genes or subset of genes in tumor samples from patients having the same type of hematological cancer which are clinically sensitive to the immunomodulatory therapy and (ii) the gene expression profile for the genes or subset of genes in first biological sample is not similar to the gene expression profile for the genes or subset of genes in tumor samples from patients having the same type of hematological cancer which are clinically insensitive to the immunomodulatory. In certain embodiments, the subset of genes comprises 3, 4, 5, 6, 7, 8, 9, 10, 11, 12, 14, 15 or more of the genes in Table 1. In some embodiments, the subset of genes comprises 2-5, 5-10, 10-15, 15-20, 20-25 or 25-30 of the genes in Table 1.

In another aspect, provided herein are methods for managing or treating a hematological cancer comprising: (a) obtaining a first biological sample from a first patient having a hematological cancer, (b) measuring the expression of the genes or a certain subset of genes set forth in Table 2, infra, in the first biological sample, and (c) comparing the gene expression profile of the genes or a subset of genes in the first biological sample to (i) the gene expression profile of the genes or subset of genes in tumor samples from patients having the same type of hematological cancer which are clinically sensitive to an immunomodulatory therapy and (ii) the gene expression of the genes or subset of genes in tumor samples from patients having the same type of hematological cancer which are clinically insensitive to the immunomodulatory therapy, and (d) administering the immunomodulatory therapy to the first patient if the gene expression profile for the genes or subset of genes in the first biological sample is similar to the gene expression profile for the genes or subset of genes in tumor samples from patients having the same type of hematological cancer which are clinically sensitive to the immunomodulatory therapy. In certain embodiments, the subset of genes comprises 3, 4, 5, 6, 7, 8, 9, 10, 11, 12, 14, 15 or more of the genes in Table 2. In some embodiments, the subset of genes comprises 2-5, 5-10, 10-15, 15-20, 20-25 or 25-30 of the genes in Table 2.

In another aspect, provided herein are methods for managing or treating a hematological cancer comprising: (a) obtaining a first biological sample from a first patient having a hematological cancer, (b) measuring the expression of the genes or a certain subset of genes set forth in Table 2, infra, in the first biological sample, and (c) comparing the gene expression profile of the genes or subset of genes in the first biological sample to (i) the gene expression profile of the genes or subset of genes in tumor samples from patients having the same type of hematological cancer which are clinically sensitive to an immunomodulatory therapy and (ii) the gene expression of the genes or subset of genes i in tumor samples from patients having the same type of hematological cancer which are clinically insensitive to the immunomodulatory therapy, and (d) administering the immunomodulatory therapy to the first patient if the gene expression profile for the genes or subset of genes in the first biological sample is not similar to the gene expression profile for the genes or subset of genes in tumor samples from patients having the same type of hematological cancer which are clinically insensitive to the immunomodulatory therapy. In certain embodiments, the subset of genes comprises 3, 4, 5, 6, 7, 8, 9, 10, 11, 12, 14, 15 or more of the genes in Table 2. In some embodiments, the subset of genes comprises 2-5, 5-10, 10-15, 15-20, 20-25 or 25-30 of the genes in Table 2.

In another aspect, provided herein are methods for managing or treating a hematological cancer comprising: (a) obtaining a first biological sample from a first patient having a hematological cancer, (b) measuring the expression of the genes or a certain subset of genes set forth in Table 2, infra, in the first biological sample, and (c) comparing the gene expression profile of the genes or subset of genes in the first biological sample to (i) the gene expression profile of the genes or subset of genes in tumor samples from patients having the same type of hematological cancer which are clinically sensitive to an immunomodulatory therapy and (ii) the gene expression of the genes or subset of genes in tumor samples from patients having the same type of hematological cancer which are clinically insensitive to the immunomodulatory therapy, and (d) administering the immunomodulatory therapy to the first patient if: (i) the gene expression profile for the genes or subset of genes in the first biological sample is similar to the gene expression profile for the genes or subset of genes in tumor samples from patients having the same type of hematological cancer which are clinically sensitive to the immunomodulatory therapy and (ii) the gene expression profile for the genes or subset of genes in first biological sample is not similar to the gene expression profile for the genes or subset of genes in tumor samples from patients having the same type of hematological cancer which are clinically insensitive to the immunomodulatory. In certain embodiments, the subset of genes comprises 3, 4, 5, 6, 7, 8, 9, 10, 1, 12, 14, 15 or more of the genes in Table 2. In some embodiments, the subset of genes comprises 2-5, 5-10, 10-15, 15-20, 20-25 or 25-30 of the genes in Table 2.

In accordance with the methods described herein, the biological sample can be any sample obtained from the patient. In certain embodiments, the biological sample is a cell sample. In other embodiments, the biological sample is whole blood sample, peripheral blood mononuclear cell sample, or tissue sample. In specific embodiments, the biological sample is a tumor sample. See Section 5.8, infra, regarding biological samples.

In accordance with the methods described herein, the level of expression of one, two, three, four, five or more of the genes in Table 1 and/or Table 2 and/or Table 3 and/or Table 4, infra, can be measured at the RNA and/or protein levels. In certain embodiments, the level of expression of the genes are measured at the RNA (e.g., mRNA) level. In other embodiments, the level of expression of the genes are measured at the protein level.

Techniques known to one skilled in the art may be used to measure the amount of an RNA transcript(s). In some embodiments, the amount of one, two, three, four, five or more RNA transcripts is measured using deep sequencing, such as ILLUMINA® RNASeq, ILLUMINA® next generation sequencing (NGS), ION TORRENT™ RNA next generation sequencing, 454™ pyrosequencing, or Sequencing by Oligo Ligation Detection (SOLID™). In other embodiments, the amount of multiple RNA transcripts is measured using a microarray and/or gene chip, such as described in Section 6, infra. In certain embodiments, the amount of one, two, three or more RNA transcripts is determined by RT-PCR. In other embodiments, the amount of one, two, three or more RNA transcripts is measured by RT-qPCR. Techniques for conducting these assays are known to one skilled in the art. See Section 5.9, infra, for examples of assays for measuring RNA transcripts.

In some embodiments, a statistical analysis or other analysis is performed on data from the assay utilized to measure an RNA transcript or protein. In some specific embodiments, p value of those RNA transcripts or proteins differentially expressed is 0.1, 0.5, 0.4, 0.3, 0.2, 0.01, 0.05, 0.001, or 0.0001. In specific embodiments, a false discovery rate (FDR) of 10%, 9%, 8%, 7%, 6%, 5%, 4%, 3%, 2% 1% or less is selected.

Techniques known to one skilled in the art may be used to measure the amount of a protein. For example, flow cytometry, immunofluorescence, enzyme-linked immunosorbent assay-based methodologies (ELISA) and similar assays known in the art. See Section 5.9, infra, for examples of assays for measuring RNA transcripts.

In another aspect, provided herein are methods for managing or treating a hematological cancer comprising: (a) obtaining a first tumor sample from a first patient having the hematological cancer, (b) measuring the proportion of dendritic cells in the first tumor sample, (c) comparing the proportion of dendritic cells in the first tumor sample with the proportion of dendritic cells in a second tumor sample from a second patient having the same type of hematological cancer, wherein the second patient's hematological cancer is clinically insensitive to treatment with an immunomodulatory therapy, and (d) administering the immunomodulatory therapy to the first patient if a higher proportion of dendritic cells in the first tumor sample is measured relative the proportion of dendritic cells in the second tumor sample.

In another aspect, provided herein are methods for managing or treating a hematological cancer comprising: (a) obtaining a first tumor sample from a first patient having the hematological cancer, (b) measuring the proportion of plasma cells in the first tumor sample, (c) comparing the proportion of plasma cells in the first tumor sample with the proportion of plasma cells in a second tumor sample from a second patient having the same type of hematological cancer, wherein the second patient's hematological cancer is clinically insensitive to treatment with an immunomodulatory therapy, and (d) administering the immunomodulatory therapy to the first patient if a higher proportion of plasma cells in the first tumor sample is measured relative the proportion of plasma cells in the second tumor sample.

In another aspect, provided herein are methods for managing or treating a hematological cancer comprising: (a) obtaining a first tumor sample from a first patient having the hematological cancer, (b) measuring the proportion of dendritic cells and plasma cells in the first tumor sample, (c) comparing the proportion of dendritic cells and plasma cells in the first tumor sample with the proportion of dendritic cells and plasma cells in a second tumor sample from a second patient having the same type of hematological cancer, wherein the second patient's hematological cancer is clinically insensitive to treatment with an immunomodulatory therapy, and (d) administering the immunomodulatory therapy to the first patient if a higher proportion of dendritic cells and plasma cells in the first tumor sample is measured relative the proportion of dendritic cells and plasma cells in the second tumor sample.

In another aspect, provided herein are methods for managing or treating a hematological cancer comprising: (a) obtaining a first tumor sample from a first patient having the hematological cancer, (b) measuring the proportion of B cells in the first tumor sample, (c) comparing the proportion of B cells in the first tumor sample with the proportion of B cells in a second tumor sample from a second patient having the same type of hematological cancer, wherein the second patient's hematological cancer is clinically insensitive to treatment with an immunomodulatory therapy, and (d) administering the immunomodulatory therapy to the first patient if a decreased proportion of B cells in the first tumor sample is measured relative the proportion of B cells in the second tumor sample.

In another aspect, provided herein are methods for managing or treating a hematological cancer comprising: (a) obtaining a first tumor sample from a first patient having the hematological cancer, (b) measuring the proportion of tumor infiltrating immune cells in the first tumor sample, (c) comparing the proportion of tumor infiltrating immune cells in the first tumor sample with the proportion of tumor infiltrating immune cells in a second tumor sample from a second patient having the same type of hematological cancer, wherein the second patient's hematological cancer is clinically insensitive to treatment with an immunomodulatory therapy, and (d) administering the immunomodulatory therapy to the first patient if a higher proportion of tumor infiltrating immune cells in the first tumor sample is measured relative the proportion of tumor infiltrating immune cells in the second tumor sample.

In another aspect, provided herein are methods for managing or treating a hematological cancer comprising: (a) obtaining a first tumor sample from a first patient having the hematological cancer, (b) measuring the proportion of NK cells in the first tumor sample, (c) comparing the proportion of NK cells in the first tumor sample with the proportion of NK cells in a second tumor sample from a second patient having the same type of hematological cancer, wherein the second patient's hematological cancer is clinically insensitive to treatment with an immunomodulatory therapy, and (d) administering the immunomodulatory therapy to the first patient if a higher proportion of NK cells in the first tumor sample is measured relative the proportion of NK cells in the second tumor sample.

In another aspect, provided herein are methods for managing or treating a hematological cancer comprising: (a) obtaining a first tumor sample from a first patient having the hematological cancer, (b) measuring the proportion of monocytes in the first tumor sample, (c) comparing the proportion of monocytes in the first tumor sample with the proportion of monocytes in a second tumor sample from a second patient having the same type of hematological cancer, wherein the second patient's hematological cancer is clinically insensitive to treatment with an immunomodulatory therapy, and (d) administering the immunomodulatory therapy to the first patient if a higher proportion of monocytes in the first tumor sample is measured relative the proportion of monocytes in the second tumor sample.

In certain embodiments of the foregoing paragraphs in this section, the second patient is a single patient. In other embodiments of the foregoing paragraphs in this section, the second patient is a population of patients. In specific embodiments, the population comprises 5, 10, 15, 20, 25, 30, 35, 40, 45, 50, 55, 60, 65, 70, 75, 80, 85, 90, 95, 100, 125, 150, 200, 225, 250, 300 or more patients.

In another aspect, provided herein are for managing or treating a hematological cancer comprising: (a) obtaining a first tumor sample from a first patient having a hematological cancer, (b) measuring the proportion of immune cells in the first tumor sample, and (c) comparing the proportion of the immune cells in the first tumor sample to (i) the proportion of the same immune cells in tumor samples from patients having the same type of hematological cancer which are clinically sensitive to an immunomodulatory therapy and (ii) the proportion of the same immune cells in tumor samples from patients having the same type of hematological cancer which are clinically insensitive to the immunomodulatory therapy, and (d) administering the immunomodulatory therapy to the first patient if the proportion of the immune cells in the first tumor sample is similar to the proportion of the same immune cells in tumor samples from patients having the same type of hematological cancer which are clinically sensitive to the immunomodulatory therapy. In some embodiments, the immune cells are subset of immune cells, such as subset of B cells. In certain embodiments, the immune cells are dendritic cells. In some embodiments, the immune cells are plasma cells. In certain embodiments, the immune cells are monocytes. In some embodiments, the immune cells are tumor infiltrating immune cells. In certain embodiments, the immune cells are T cells. In some embodiments, the immune cells are B cells. In certain embodiments, the immune cells are NK cells. In some embodiments, the immune cells are two, three or more subsets of immune cells, such as two more types of T cells (e.g., CD4+ and CD8+ T cells). In some embodiments, the proportion of different populations of immune cells in the first tumor sample are compared to (i) the proportion of the same populations of immune cells in the tumor samples from patients having the same type of hematological cancer which are clinically sensitive to the immunomodulatory therapy and (ii) the proportion of the same populations of immune cells in the tumor samples from patients having the same type of hematological cancer which are clinically insensitive to the immunomodulatory therapy.

In another aspect, provided herein are for managing or treating a hematological cancer comprising: (a) obtaining a first tumor sample from a first patient having a hematological cancer, (b) measuring the proportion of immune cells in the first tumor sample, and (c) comparing the proportion of the immune cells in the first tumor sample to (i) the proportion of the same immune cells in tumor samples from patients having the same type of hematological cancer which are clinically sensitive to an immunomodulatory therapy and (ii) the proportion of the same immune cells in tumor samples from patients having the same type of hematological cancer which are clinically insensitive to the immunomodulatory therapy, and (d) administering the immunomodulatory therapy to the first patient if the proportion of the immune cells in the first tumor sample is not similar to the proportion of the same immune cells in tumor samples from patients having the same type of hematological cancer which are clinically insensitive to the immunomodulatory therapy. In some embodiments, the immune cells are subset of immune cells, such as subset of B cells. In certain embodiments, the immune cells are dendritic cells. In some embodiments, the immune cells are plasma cells. In certain embodiments, the immune cells are monocytes. In some embodiments, the immune cells are tumor infiltrating immune cells. In certain embodiments, the immune cells are T cells. In some embodiments, the immune cells are B cells. In certain embodiments, the immune cells are NK cells. In some embodiments, the immune cells are two, three or more subsets of immune cells, such as two more types of T cells (e.g., CD4+ and CD8+ T cells). In some embodiments, the proportion of different populations of immune cells in the first tumor sample are compared to (i) the proportion of the same populations of immune cells in the tumor samples from patients having the same type of hematological cancer which are clinically sensitive to the immunomodulatory therapy and (ii) the proportion of the same populations of immune cells in the tumor samples from patients having the same type of hematological cancer which are clinically insensitive to the immunomodulatory therapy.

In another aspect, provided herein are for managing or treating a hematological cancer comprising: (a) obtaining a first tumor sample from a first patient having a hematological cancer, (b) measuring the proportion of immune cells in the first tumor sample, and (c) comparing the proportion of the immune cells in the first tumor sample to (i) the proportion of the same immune cells in tumor samples from patients having the same type of hematological cancer which are clinically sensitive to an immunomodulatory therapy and (ii) the proportion of the same immune cells in tumor samples from patients having the same type of hematological cancer which are clinically insensitive to the immunomodulatory therapy, and (d) administering the immunomodulatory therapy to the first patient if the proportion of the immune cells in the first tumor sample is (i) similar to the proportion of the same immune cells in tumor samples from patients having the same type of hematological cancer which are clinically sensitive to the immunomodulatory therapy, and (ii) not similar to the proportion of the same immune cells in tumor samples from patients having the same type of hematological cancer which are clinically insensitive to the immunomodulatory therapy. In some embodiments, the immune cells are subset of immune cells, such as subset of B cells. In certain embodiments, the immune cells are dendritic cells. In some embodiments, the immune cells are plasma cells. In certain embodiments, the immune cells are monocytes. In some embodiments, the immune cells are tumor infiltrating immune cells. In certain embodiments, the immune cells are T cells. In some embodiments, the immune cells are B cells. In certain embodiments, the immune cells are NK cells. In some embodiments, the immune cells are two, three or more subsets of immune cells, such as two more types of T cells (e.g., CD4+ and CD8+ T cells). In some embodiments, the proportion of different populations of immune cells in the first tumor sample are compared to (i) the proportion of the same populations of immune cells in the tumor samples from patients having the same type of hematological cancer which are clinically sensitive to the immunomodulatory therapy and (ii) the proportion of the same populations of immune cells in the tumor samples from patients having the same type of hematological cancer which are clinically insensitive to the immunomodulatory therapy.

In addition to measuring the cells (e.g., dendritic cells and plasma cells) in a tumor sample from a patient, levels of expression of genes (e.g., one, two, three, four, five or more of the genes in Table 1 and/or Table 2 and/or Table 3 and/or Table 4) may be assessed. In specific embodiments, the methods set forth for measuring gene expression supra, are combined with the methods set forth for measuring a proportion of cells to determine if an immunomodulatory therapy is to be administered to a patient with a hematological cancer.

Techniques known to one skilled in the art may be used to measure the proportion of cells in a tumor sample. In certain embodiments, the proportion of cells is measured by flow cytometry, immunofluorescence, enzyme-linked immunosorbent assay-based methodologies (ELISA) and similar assays known in the art. In other embodiments, the proportion of cells is measured by inference from gene expression profiles.

In accordance with the methods described herein, the immunomodulatory therapy can be any therapy that modulates the immune system or immune response. Examples of immunomodulatory therapies are provided in Section 5.6, infra. In a specific embodiment, the immunomodulatory therapy is lenalidomide (Revlimid®).

In specific embodiments, an immunomodulatory therapy is administered to a hematological cancer patient in the form of a pharmaceutical composition. In a specific embodiment, an pharmaceutical composition administered to a hematological cancer patient comprising an immunomodulatory therapy and a pharmaceutically acceptable carrier or excipient. In certain embodiments, the pharmaceutical composition may comprise an additional therapy, such as described in Section 5.7, infra. The dosage form of the pharmaceutical composition will vary depending upon the route of administration. The immunomodulatory therapy or a pharmaceutical composition thereof may be administered by any route of administration, such as oral, mucosal, parenteral (e.g., subcutaneous, intravenous, bolus injection, intramuscular), topical, transdermal, or transcutaneous. In a specific embodiment, the immunomodulatory therapy or a pharmaceutical composition thereof is orally administered to a hematological cancer patient.

In accordance with the methods described herein, the dose of an immunomodulatory therapy administered to a patient varies depending on a variety of factors, such as the health and age of the patient. In certain embodiments, in accordance with the methods described herein the patient is administered a dose of 0.01 mg to 1000 mg of an immunomodulatory therapy. In certain embodiments, in accordance with the methods described herein the patient is administered a dose of 0.01 mg to 500 mg of an immunomodulatory therapy. In certain embodiments, in accordance with the methods described herein the patient is administered a dose of 0.01 mg to 100 mg of an immunomodulatory therapy. In some embodiments, in accordance with the methods described herein the patient is administered a dose of 0.1 mg to 500 mg of an immunomodulatory therapy. In some embodiments, in accordance with the methods described herein the patient is administered a dose of 0.01 mg to 500 mg of an immunomodulatory therapy. In some embodiments, in accordance with the methods described herein the patient is administered a dose of 1 mg to 500 mg of an immunomodulatory therapy. In certain embodiments, in accordance with the methods described herein the patient is administered a dose of 0.1 mg to 100 mg of an immunomodulatory therapy. In certain embodiments, in accordance with the methods described herein the patient is administered a dose of 1 mg to 100 mg of an immunomodulatory therapy. In some embodiments, in accordance with the methods described herein the patient is administered a dose of 1 mg to 50 mg of an immunomodulatory therapy. In some embodiments, in accordance with the methods described herein the patient is administered a dose of 1 mg to 100 mg of an immunomodulatory therapy. In some embodiments, in accordance with the methods described herein the patient is administered a dose of 1 mg to 500 mg of an immunomodulatory therapy. In some embodiments, in accordance with the methods described herein the patient is administered a dose of 1 mg to 1000 mg of an immunomodulatory therapy. The dose of the immunomodulatory therapy can be administered once, twice or three times per day. The dose of the immunomodulatory therapy can be administered every other day, every two days, every three days, every four days, every five days, every six days, or once per week. Specific doses per day include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg per day. The dose of the immunomodulatory therapy is administered in accordance with the label for the therapy. The immunomodulatory therapy can be lenalidomide (Revlimid®), or its pharmaceutically acceptable salt, solvate, hydrate or stereoisomer, and it is administered at a dose of 1 to 50 mg per day, or anything in between, or 25 mg per day.

In accordance with the methods described herein, the hematological cancer can be any hematological cancer. Examples of hematological cancers can be found in Section 5.5, infra. In a specific embodiment, the hematological cancer is a lymphoma. In another specific embodiment, the hematological cancer is a non-Hodgkin's lymphoma. In yet another embodiment, the hematological cancer is a diffuse large B-cell lymphoma (DLBCL). In certain embodiments, the DLBCL is a germinal center B-cell-like DLBCL. In other embodiments, the DLBCL is an activated B-cell-like DLBCL.

In certain embodiments, the methods of managing or treating a hematological cancer involve the administration of another therapy. In some embodiments, the other therapy is to alleviate pain or one or more other symptoms associated with the hematological cancer. Examples of other therapies that may be used in combination with an immunomodulatory therapy are disclosed in Section 5.7, infra. In certain embodiments, one or more of the following additional active ingredients are administered in combination with an immunomodulatory therapy in accordance with the methods described herein: oblimersen, melphalan, G-CSF, GM-CSF, EPO, a cox-2 inhibitor, topotecan, pentoxifylline, ciprofloxacin, taxotere, iritotecan, dexamethasone, doxorubicin, vincristine, IL 2, IFN, dacarbazine, Ara-C, vinorelbine and/or isotretinoin. In a specific embodiment, chemotherapeutic agents, such as cyclohexamide, hydroxydaunorubicin, oncovin, and prednisone (CHOP), are used in combination with an immunomodulatory therapy, such as lenalidomide, in accordance with the methods described herein. In another specific embodiment, rituximab is used in combination with an immunomodulatory therapy, such as lenalidomide. In another specific embodiment, CHOP and rituximab are used in combination with an immunomodulatory therapy, such as lenalidomide.

### 5.5 Hematological Cancers

In some embodiments, the hematological cancer is a lymphoma. In other embodiments, the hematological cancer is a leukemia. In one embodiment, the hematological cancer is multiple myeloma. In another embodiment, the hematological cancer is chronic lymphocytic leukemia (CLL). In another embodiment, the hematological cancer is myelodysplastic syndrome, an acute leukemia, e.g., acute T cell leukemia, acute myelogenous leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), or acute biphenotypic leukemia; a chronic leukemia, e.g., chronic myeloid lymphoma, chronic myclogenous leukemia (CML), chronic monocytic leukemia, small lymphocytic lymphoma, or B-cell prolymphocytic leukemia; hairy cell lymphoma; T-cell prolymphocytic leukemia; or a lymphoma, e.g, histiocytic lymphoma, lymphoplasmacytic lymphoma (e.g., Waldenström macroglobulinemia), splenic marginal zone lymphoma, plasma cell neoplasm (e.g., plasma cell myeloma, plasmacytoma, a monoclonal immunoglobulin deposition disease, or a heavy chain disease), extranodal marginal zone B cell lymphoma (MALT lymphoma), nodal marginal zone B cell lymphoma (NMZL), follicular lymphoma, mantle cell lymphoma, diffuse large B cell lymphoma, mediastinal (thymic) large B cell lymphoma, intravascular large B cell lymphoma, primary effusion lymphoma, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides (Sezary syndrome), a primary cutaneous CD30-positive T cell lymphoproliferative disorder (e.g., primary cutaneous anaplastic large cell lymphoma or lymphomatoid papulosis), angioimmunoblastic T cell lymphoma, peripheral T cell lymphoma, unspecified, anaplastic large cell lymphoma, a Hodgkin's lymphoma or a nodular lymphocyte-predominant Hodgkin's lymphoma.

In a specific embodiment, the hematological cancer is DLBCL. In another specific embodiment, the hematological cancer is an activated B-cell-like DLBCL. In another specific embodiment, the hematological cancer is a germinal center B-cell-like DLBCL.

### 5.6 Immunomodulatory Therapies

Immunomodulatory therapies described in the methods provided herein include compounds known as “IMiDs®” (Celgene Corporation), a group of compounds that can be useful to treat several types of human diseases, including certain cancers.

As used herein and unless otherwise indicated, the terms “immunomodulatory compound”, “immunomodulatory agent” and “immunomodulatory therapy” are used interchangeably, and can encompass certain small organic molecules that inhibit LPS induced monocyte TNF-α, IL-1B, IL-12, IL-6, MIP-1α, MCP-1, GM-CSF, G-CSF, and COX-2 production. These compounds can be prepared synthetically, or can be obtained commercially.

Exemplary immunomodulating compounds include but are not limited to N-{[2-(2,6-dioxo(3-piperidyl)-1,3-dioxoisoindolin-4-yl]methyl}cyclopropyl-carboxamide; 3-[2-(2,6-dioxo-piperidin-3-yl)-1,3-dioxo-2,3-dihydro-1H-isoindol-4-ylmethyl]-1,1-dimethyl-urea; (−)-3-(3,4-Dimethoxy-phenyl)-3-(1-oxo-1,3-dihydro-isoindol-2-yl)-propionamide; (+)-3-(3,4-Dimethoxy-phenyl)-3-(1-oxo-1,3-dihydro-isoindol-2-yl)-propionamide; (−)-{2-[1-(3-ethoxy-4-methoxyphenyl)-2-methylsulfonylethyl]-4-acetylaminoisoindoline-1,3-dione}; (+)-{2-[1-(3-ethoxy-4-methoxyphenyl)-2-methylsulfonylethyl]-4-acetylaminoisoindoline-1,3-dione}; Difluoro-methoxy SelCIDs; 1-phthalimido-1-(3,4-diethoxyphenyl)ethane; 3-(3,4-dimethoxyphenyl)-3-(3,5-dimethoxyphenyl)acrylo nitrile; 1-oxo-2-(2,6-dioxopiperidin-3-yl)-4-aminoisoindoline; 1,3-dioxo-2-(2,6-dioxopiperidin-3-yl)-4-aminoisoindoline; 4-amino-2-(3-methyl-2,6-dioxo-piperidine-3-yl)-isoindole-1,3-dione; 3-(3-acetoamidophthalimido)-3-(3-ethoxy-4-methoxyphenyl)-N-hydroxypropionamide; 1-oxo-2-(2,6-dioxopiperidin-3-yl)-4-methylisoindoline; Cyclopropyl-N-{2-[(1S)-1-(3-ethoxy-4-methoxyphenyl)-2-(methylsulfonyl)ethyl]-3-oxoisoindoline-4-yl}carboxamide; Substituted 2-(3-hydroxy-2,6-dioxopiperidin-5-yl) isoindoline; N-[2-(2,6-Dioxo-piperidin-3-yl)-1,3-dioxo-2,3-dihydro-1H-isoindol-5-ylmethyl]-4-trifluoromethoxybenzamide; (S)-4-chloro-N-((2-(3-methyl-2,6-dioxopiperidin-3-yl)-1,3-dioxoisoindolin-5-yl)methyl) benzamide; Pyridine-2-carboxylic acid [2-[(3S)-3-methyl-2,6-dioxo-piperidin-3-yl]-1,3-dioxo-2,3-dihydro-1H-isoindol-5-ylmethyl]-amide; (S)—N-((2-(3-methyl-2,6-dioxopiperidin-3-yl)-1,3-dioxoisoindolin-5-yl)methyl)-4-(trifluoromethyl)benzamide; 3-(2,5-dimethyl-4-oxo-4H-quinazolin-3-yl)-piperidine-2,6-dione, and the like. In a specific embodiment, the immunomodulatory compound is 3-(4-amino-1-oxo-1,3-dihydro-isoindol-2-yl)-piperidine-2,6-dione, or a salt, solvate or hydrate thereof.

Immunomodulatory compounds disclosed herein may enhance the degradation of TNF-α mRNA. Immunomodulatory compounds disclosed herein may also be potent co-stimulators of T cells and increase cell proliferation dramatically in a dose dependent manner. Immunomodulatory compounds disclosed herein may also have a greater co-stimulatory effect on the CD8+ T cell subset than on the CD4+ T cell subset. Immunomodulatory compounds disclosed herein may be capable of acting both indirectly through cytokine activation and directly on Natural Killer (“NK”) cells and Natural Killer T (“NKT”) cells, and increase the NK cells' ability to produce beneficial cytokines such as, but not limited to, IFN-γ, and to enhance NK and NKT cell cytotoxic activity.

Specific examples of immunomodulatory compounds include cyano and carboxy derivatives of substituted styrenes such as those disclosed in U.S. Pat. No. 5,929,117; 1-oxo-2-(2,6-dioxo-3-fluoropiperidin-3yl) isoindolines and 1,3-dioxo-2-(2,6-dioxo-3-fluoropiperidine-3-yl) isoindolines such as those described in U.S. Pat. Nos. 5,874,448 and 5,955,476; the tetra substituted 2-(2,6-dioxopiperdin-3-yl)-1-oxoisoindolines described in U.S. Pat. No. 5,798,368; 1-oxo and 1,3-dioxo-2-(2,6-dioxopiperidin-3-yl) isoindolines (e.g., 4-methyl derivatives of thalidomide), substituted 2-(2,6-dioxopiperidin-3-yl) phthalimides and substituted 2-(2,6-dioxopiperidin-3-yl)-1-oxoisoindoles including, but not limited to, those disclosed in U.S. Pat. Nos. 5,635,517, 6,281,230, 6,316,471, 6,403,613, 6,476,052 and 6,555,554; 1-oxo and 1,3-dioxoisoindolines substituted in the 4- or 5-position of the indoline ring (e.g., 4-(4-amino-1,3-dioxoisoindoline-2-yl)-4-carbamoylbutanoic acid) described in U.S. Pat. No. 6,380,239; isoindoline-1-one and isoindoline-1,3-dione substituted in the 2-position with 2,6-dioxo-3-hydroxypiperidin-5-yl (e.g., 2-(2,6-dioxo-3-hydroxy-5-fluoropiperidin-5-yl)-4-aminoisoindolin-1-one) described in U.S. Pat. No. 6,458,810; a class of non-polypeptide cyclic amides disclosed in U.S. Pat. Nos. 5,698,579 and 5,877,200; and isoindole-imide compounds such as those described in U.S. patent publication no. 2003/0045552 published on Mar. 6, 2003, U.S. patent publication no. 2003/0096841 published on May 22, 2003, and International Application No. PCT/US01/50401 (International Publication No. WO 02/059106). US patent publication no. 2006/0205787 describes 4-amino-2-(3-methyl-2,6-dioxopiperidin-3-yl)-isoindole-1,3-dione compositions. US patent publication no. 2007/0049618 describes isoindole-imide compounds. The entireties of each of the patents and patent applications identified herein are incorporated by reference. In one embodiment, immunomodulatory compounds do not include thalidomide.

Various immunomodulatory compounds disclosed herein contain one or more chiral centers, and can exist as racemic mixtures of enantiomers or mixtures of diastereomers. Thus, also provided herein is the use of stereomerically pure forms of such compounds, as well as the use of mixtures of those forms. For example, mixtures comprising equal or unequal amounts of the enantiomers of a particular immunomodulatory compounds may be used. These isomers may be asymmetrically synthesized or resolved using standard techniques such as chiral columns or chiral resolving agents. See, e.g., Jacques. J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

Immunomodulatory compounds provided herein include, but are not limited to, 1-oxo- and 1,3 dioxo-2-(2,6-dioxopiperidin-3-yl) isoindolines substituted with amino in the benzo ring as described in U.S. Pat. No. 5,635,517 which is incorporated herein by reference.

These compounds have the structure I:

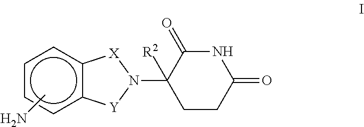

in which one of X and Y is C═O, the other of X and Y is C═O or CH₂, and R² is hydrogen or lower alkyl, in particular methyl. Specific immunomodulatory compounds include, but are not limited to:

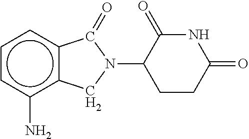

1-oxo-2-(2,6-dioxopiperidin-3-yl)-4-aminoisoindoline;

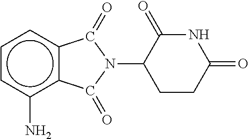

1,3-dioxo-2-(2,6-dioxopiperidin-3-yl)-4-aminoisoindoline; and

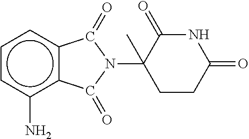

1,3-dioxo-2-(3-methyl-2,6-dioxopiperidin-3-yl)-4-aminoisoindole, and optically pure isomers thereof.

The compounds can be obtained via standard, synthetic methods (see e.g., U.S. Pat. No. 5,635,517, incorporated herein by reference). The compounds are also available from Celgene Corporation, Warren, N.J.

Other specific immunomodulatory compounds belong to a class of substituted 2-(2,6-dioxopiperidin-3-yl) phthalimides and substituted 2-(2,6-dioxopiperidin-3-yl)-1-oxoisoindoles, such as those described in U.S. Pat. Nos. 6,281,230; 6,316,471; 6,335,349; and 6,476,052, and International Patent Application No. PCT/US97/13375 (International Publication No. WO 98/03502), each of which is incorporated herein by reference. Representative compounds are of formula:

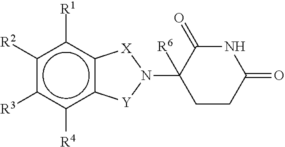

in which:
one of X and Y is C═O and the other of X and Y is C═O or CH₂;
(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;
R⁵ is hydrogen or alkyl of 1 to 8 carbon atoms;
R⁶ is hydrogen, alkyl of 1 to 8 carbon atoms, benzyl, or halo;
provided that R⁶ is other than hydrogen if X and Y are C═O and (i) each of R¹, R², R³, and R⁴ is fluoro or (ii) one of R¹, R², R³, or R⁴ is amino.

Compounds representative of this class are of the formulas:

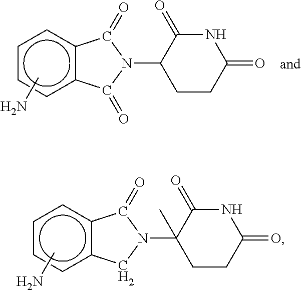

wherein R¹ is hydrogen or methyl. In a separate embodiment, provided herein is the use of enantiomerically pure forms (e.g. optically pure (R) or (S) enantiomers) of these compounds.

Still other specific immunomodulatory compounds disclosed herein belong to a class of isoindole-imides disclosed in U.S. Pat. No. 7,091,353, U.S. Patent Publication No. 2003/0045552, and International Application No. PCT/US01/50401 (International Publication No. WO 02/059106), each of which are incorporated herein by reference. Representative compounds are of formula II:

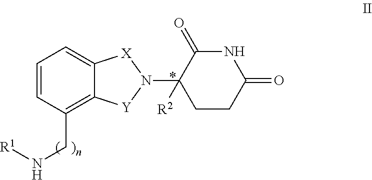

and pharmaceutically acceptable salts, hydrates, solvates, clathrates, enantiomers, diastereomers, racemates, and mixtures of stereoisomers thereof, wherein:
one of X and Y is C═O and the other is CH₂ or C═O;
R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(S)R³, C(O)OR⁴, (C₁-C₈)alkyl-N(R⁶)2, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;
R² is H, F, benzyl, (C₁-C₈)alkyl, (C₂-C₈)alkenyl, or (C₂-C₈)alkynyl;
R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₀-C₈)alkyl-N(R⁶)2, (C₁-C₈)alkyl-OR⁵, (C₁-C₈)alkyl-C(O)OR⁵, (C₁-C₈)alkyl-O(CO)R⁵, or C(O)OR⁵;
R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;
R⁵ is (C₁-C₈)alkyl, (C₂-C₈)alkenyl, (C₂-C₈)alkynyl, benzyl, aryl, or (C₂-C₅)heteroaryl;
each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group;
n is 0 or 1; and
* represents a chiral-carbon center.

In specific compounds of formula II, when n is 0 then R¹ is (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(O)OR⁴, (C₁-C₈)alkyl-N(R⁶)₂, (C₁-C₈)alkyl-OR⁵, (C₁-C₈)alkyl-C(O)OR⁵, C(S)NHR³, or (C₁-C₈)alkyl-O(CO)R⁵;

R² is H or (C₁-C₈)alkyl; and
R³ is (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₅-C₈)alkyl-N(R⁶)2; (C₀-C₈)alkyl-NH—C(O)O—R⁵; (C₁-C₈)alkyl-OR⁵, (C₁-C₈)alkyl-C(O)OR⁵, (C₁-C₈)alkyl-O(CO)R⁵, or C(O)OR⁵;
and the other variables have the same definitions.

In other specific compounds of formula II, R² is H or (C₁-C₄)alkyl.

In other specific compounds of formula II, R¹ is (C₁-C₈)alkyl or benzyl.

In other specific compounds of formula II, R¹ is H, (C₁-C₈)alkyl, benzyl, CH₂OCH₃, CH₂CH₂OCH₃, or

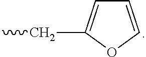

In another embodiment of the compounds of formula II, R¹ is

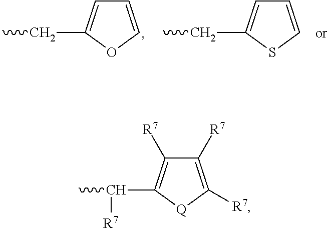

wherein Q is O or S, and each occurrence of R⁷ is independently H, (C₁-C₈)alkyl, (C₃-C₇)cycloalkyl, (C₂-C₈)alkenyl, (C₂-C₈)alkynyl, benzyl, aryl, halogen, (C₀-C₄)alkyl-(C₁-C₆)heterocycloalkyl, (C₀-C₄)alkyl-(C₂-C₅)heteroaryl, (C₀-C₈)alkyl-N(R⁶)2, (C₁-C₈)alkyl-OR⁵, (C₁-C₈)alkyl-C(O)OR⁵, (C₁-C₈)alkyl-O(CO)R⁵, or C(O)OR⁵, or adjacent occurrences of R⁷ can be taken together to form a bicyclic alkyl or aryl ring.

In other specific compounds of formula II, R¹ is C(O)R³.

In other specific compounds of formula II, R³ is (C₀-C₄)alkyl-(C₂-C₅)heteroaryl, (C₁-C₈)alkyl, aryl, or (C₀-C₄)alkyl-OR⁵.

In other specific compounds of formula II, heteroaryl is pyridyl, furyl, or thienyl.

In other specific compounds of formula II, R¹ is C(O)OR⁴.

In other specific compounds of formula II, the H of C(O)NHC(O) can be replaced with (C₁-C₄)alkyl, aryl, or benzyl.

Further examples of the compounds in this class include, but are not limited to: [2-(2,6-dioxo-piperidin-3-yl)-1,3-dioxo-2,3-dihydro-1H-isoindol-4-ylmethyl]-amide; (2-(2,6-dioxo-piperidin-3-yl)-1,3-dioxo-2,3-dihydro-1H-isoindol-4-ylmethyl)-carbamic acid tert-butyl ester; 4-(aminomethyl)-2-(2,6-dioxo(3-piperidyl))-isoindoline-1,3-dione; N-(2-(2,6-dioxo-piperidin-3-yl)-1,3-dioxo-2,3-dihydro-1H-isoindol-4-ylmethyl)-acetamide; N-{(2-(2,6-dioxo(3-piperidyl)-1,3-dioxoisoindolin-4-yl)methyl}cyclopropyl-carboxamide; 2-chloro-N-{(2-(2,6-dioxo(3-piperidyl))-1,3-dioxoisoindolin-4-yl)methyl}acetamide; N-(2-(2,6-dioxo(3-piperidyl))-1,3-dioxoisoindolin-4-yl)-3-pyridylcarboxamide; 3-{1-oxo-4-(benzylamino)isoindolin-2-yl}piperidine-2,6-dione; 2-(2,6-dioxo(3-piperidyl))-4-(benzylamino)isoindoline-1,3-dione; N-{(2-(2,6-dioxo(3-piperidyl))-1,3-dioxoisoindolin-4-yl)methyl}propanamide; N-{(2-(2,6-dioxo(3-piperidyl))-1,3-dioxoisoindolin-4-yl)methyl}-3-pyridylcarboxamide; N-{(2-(2,6-dioxo(3-piperidyl))-1,3-dioxoisoindolin-4-yl)methyl}heptanamide; N-{(2-(2,6-dioxo(3-piperidyl))-1,3-dioxoisoindolin-4-yl)methyl}-2-furylcarboxamide; {N-(2-(2,6-dioxo(3-piperidyl))-1,3-dioxoisoindolin-4-yl)carbamoyl}methyl acetate; N-(2-(2,6-dioxo(3-piperidyl))-1,3-dioxoisoindolin-4-yl)pentanamide; N-(2-(2,6-dioxo(3-piperidyl))-1,3-dioxoisoindolin-4-yl)-2-thienylcarboxamide; N-{[2-(2,6-dioxo(3-piperidyl))-1,3-dioxoisoindolin-4-yl]methyl}(butylamino)carboxamide; N-{[2-(2,6-dioxo(3-piperidyl))-1,3-dioxoisoindolin-4-yl]methyl}(octylamino)carboxamide; and N-{[2-(2,6-dioxo(3-piperidyl))-1,3-dioxoisoindolin-4-yl]methyl}(benzylamino)carboxamide.

Still other specific immunomodulatory compounds disclosed herein belong to a class of isoindole-imides disclosed in U.S. Patent Application Publication Nos. US 2002/0045643, International Publication No. WO 98/54170, and U.S. Pat. No. 6,395,754, each of which is incorporated herein by reference. Representative compounds are of formula III:

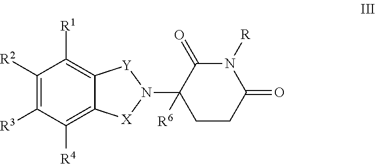

and pharmaceutically acceptable salts, hydrates, solvates, clathrates, enantiomers, diastereomers, racemates, and mixtures of stereoisomers thereof, wherein:
one of X and Y is C═O and the other is CH₂ or C═O;

### R is H or CH₂OCOR′;

(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;
R⁵ is hydrogen or alkyl of 1 to 8 carbons
R⁶ hydrogen, alkyl of 1 to 8 carbon atoms, benzo, chloro, or fluoro;
R′ is R⁷—CHR¹⁰—N(R⁸R⁹);
R⁷ is m-phenylene or p-phenylene or -(CnH2n)- in which n has a value of 0 to 4;
each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S—, or —NH—;
R¹⁰ is hydrogen, alkyl of to 8 carbon atoms, or phenyl; and
* represents a chiral-carbon center.

Other representative compounds are of formula:

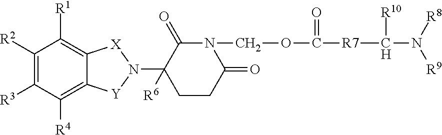

wherein:
one of X and Y is C═O and the other of X and Y is C═O or CH₂;

(i) each of R¹, R², R³, or R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzo, chloro, or fluoro;

R⁷ is m-phenylene or p-phenylene or -(CnH2n)- in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S—, or —NH—; and

R¹⁰ is hydrogen, alkyl of to 8 carbon atoms, or phenyl.

Other representative compounds are of formula:

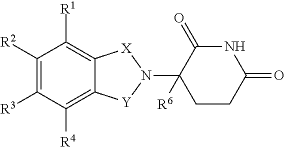

in which

one of X and Y is C═O and the other of X and Y is C═O or CH₂;

each of R¹, R², R³, and R⁴, independently of the others, is halo, alkyl of 1 to 4 carbon atoms, or alkoxy of 1 to 4 carbon atoms or (ii) one of R¹, R², R³, and R⁴ is nitro or protected amino and the remaining of R¹, R², R³, and R⁴ are hydrogen; and

R⁶ is hydrogen, alkyl of 1 to 8 carbon atoms, benzo, chloro, or fluoro.

Other representative compounds are of formula:

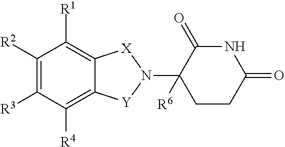

in which:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen, alkyl of 1 to 8 carbon atoms, or CO—R⁷—CH(R¹⁰)NR⁸R⁹ in which each of R⁷, R⁸, R⁹, and R¹⁰ is as herein defined; and

R⁶ is alkyl of 1 to 8 carbon atoms, benzo, chloro, or fluoro.

Specific examples of the compounds are of formula:

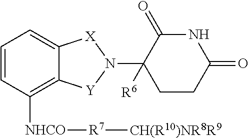

in which:

one of X and Y is C═O and the other of X and Y is C═O or CH₂;

R⁶ is hydrogen, alkyl of 1 to 8 carbon atoms, benzyl, chloro, or fluoro;

R⁷ is m-phenylene, p-phenylene or -(CnH2n)- in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S— or —NH—; and

R¹⁰ is hydrogen, alkyl of 1 to 8 carbon atoms, or phenyl.

Other specific immunomodulatory compounds are 1-oxo-2-(2,6-dioxo-3-fluoropiperidin-3yl) isoindolines and 1,3-dioxo-2-(2,6-dioxo-3-fluoropiperidine-3-yl) isoindolines such as those described in U.S. Pat. Nos. 5,874,448 and 5,955,476, each of which is incorporated herein by reference. Representative compounds are of formula:

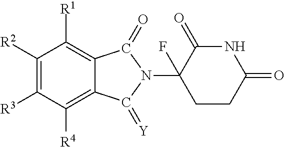

wherein:
Y is oxygen or H₂ and
each of R¹, R², R³, and R⁴, independently of the others, is hydrogen, halo, alkyl of 1 to 4 carbon atoms, alkoxy of 1 to 4 carbon atoms, or amino.

Other specific immunomodulatory compounds are the tetra substituted 2-(2,6-dioxopiperdin-3-yl)-1-oxoisoindolines described in U.S. Pat. No. 5,798,368, which is incorporated herein by reference. Representative compounds are of formula:

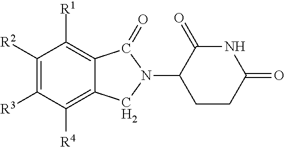

wherein each of R¹, R², R³, and R⁴, independently of the others, is halo, alkyl of 1 to 4 carbon atoms, or alkoxy of 1 to 4 carbon atoms.

Other specific immunomodulatory compounds are 1-oxo and 1,3-dioxo-2-(2,6-dioxopiperidin-3-yl) isoindolines disclosed in U.S. Pat. No. 6,403,613, which is incorporated herein by reference. Representative compounds are of formula:

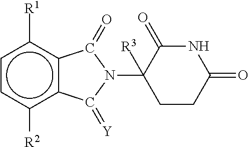

in which

Y is oxygen or H₂,

a first of R¹ and R² is halo, alkyl, alkoxy, alkylamino, dialkylamino, cyano, or carbamoyl, the second of R¹ and R², independently of the first, is hydrogen, halo, alkyl, alkoxy, alkylamino, dialkylamino, cyano, or carbamoyl, and

R³ is hydrogen, alkyl, or benzyl.

Specific examples of the compounds are of formula:

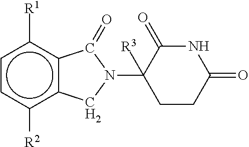

wherein
a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl;
the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl; and R³ is hydrogen, alkyl of from 1 to 4 carbon atoms, or benzyl. Specific examples include, but are not limited to, 1-oxo-2-(2,6-dioxopiperidin-3-yl)-4-methylisoindoline.

Other representative compounds are of formula:

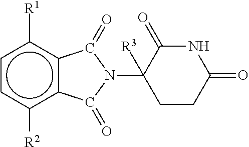

wherein:
a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl;
the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl; and
R³ is hydrogen, alkyl of from 1 to 4 carbon atoms, or benzyl.

Other specific immunomodulatory compounds disclosed herein are 1-oxo and 1,3-dioxoisoindolines substituted in the 4- or 5-position of the indoline ring described in U.S. Pat. No. 6,380,239 and U.S. Pat. No. 7,244,759, both of which are incorporated herein by reference. Representative compounds are of formula:

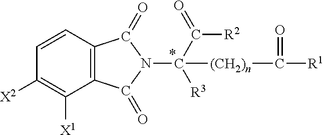

in which the carbon atom designated C* constitutes a center of chirality (when n is not zero and R¹ is not the same as R²); one of X¹ and X² is amino, nitro, alkyl of one to six carbons, or NH—Z, and the other of X¹ or X² is hydrogen; each of R¹ and R² independent of the other, is hydroxy or NH—Z; R³ is hydrogen, alkyl of one to six carbons, halo, or haloalkyl; Z is hydrogen, aryl, alkyl of one to six carbons, formyl, or acyl of one to six carbons; and n has a value of 0, 1, or 2; provided that if X¹ is amino, and n is 1 or 2, then R¹ and R² are not both hydroxy; and the salts thereof.

Further representative compounds are of formula:

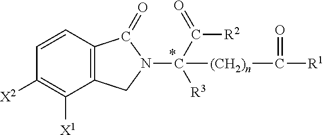

in which the carbon atom designated C* constitutes a center of chirality when n is not zero and R¹ is not R²; one of X¹ and X² is amino, nitro, alkyl of one to six carbons, or NH—Z, and the other of X¹ or X² is hydrogen; each of R¹ and R² independent of the other, is hydroxy or NH—Z; R³ is alkyl of one to six carbons, halo, or hydrogen; Z is hydrogen, aryl or an alkyl or acyl of one to six carbons; and n has a value of 0, 1, or 2.

Specific examples include, but are not limited to, 2-(4-amino-1-oxo-1,3-dihydro-isoindol-2-yl)-4-carbamoyl-butyric acid and 4-(4-amino-1-oxo-1,3-dihydro-isoindol-2-yl)-4-cabamoyl-butyric acid, which have the following structures, respectively, and pharmaceutically acceptable salts, solvates, prodrugs, and stereoisomers thereof:

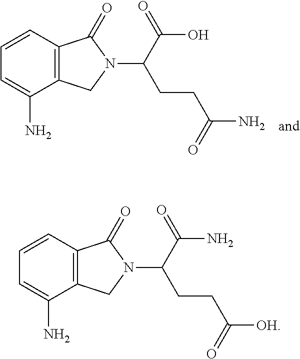 [SMILES: NC(=O)CCC(C(=O)O)N1Cc2c(N)cccc2C1=O ; NC(=O)C(CCC(=O)O)N1Cc2c(N)cccc2C1=O]

Other representative compounds are of formula:

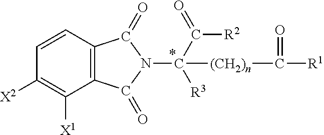

in which the carbon atom designated C* constitutes a center of chirality when n is not zero and R¹ is not R²; one of X¹ and X² is amino, nitro, alkyl of one to six carbons, or NH—Z, and the other of X¹ or X² is hydrogen; each of R¹ and R² independent of the other, is hydroxy or NH—Z; R³ is alkyl of one to six carbons, halo, or hydrogen; Z is hydrogen, aryl, or an alkyl or acyl of one to six carbons; and n has a value of 0, 1, or 2; and the salts thereof.

Specific examples include, but are not limited to, 4-carbamoyl-4-{4-[(furan-2-yl-methyl)-amino]-1,3-dioxo-1,3-dihydro-isoindol-2-yl}-butyric acid, 4-carbamoyl-2-{4-[(furan-2-yl-methyl)-amino]-1,3-dioxo-1,3-dihydro-isoindol-2-yl}-butyric acid, 2-{4-[(furan-2-yl-methyl)-amino]-1,3-dioxo-1,3-dihydro-isoindol-2-yl}-4-phenylcarbamoyl-butyric acid, and 2-{4-[(furan-2-yl-methyl)-amino]-1,3-dioxo-1,3-dihydro-isoindol-2-yl}-pentanedioic acid, which have the following structures, respectively, and pharmaceutically acceptable salts, solvate, prodrugs, and stereoisomers thereof:

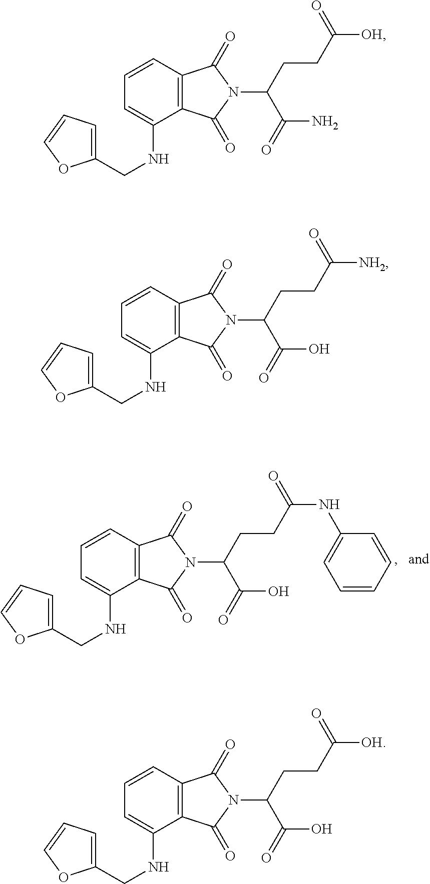 [SMILES: NC(=O)C(CCC(=O)O)N1C(=O)c2cccc(NCc3ccco3)c2C1=O ; NC(=O)CCC(C(=O)O)N1C(=O)c2cccc(NCc3ccco3)c2C1=O ; O=C(CCC(C(=O)O)N1C(=O)c2cccc(NCc3ccco3)c2C1=O)Nc1ccccc1 ; O=C(O)CCC(C(=O)O)N1C(=O)c2cccc(NCc3ccco3)c2C1=O]

Other specific examples of the compounds are of formula:

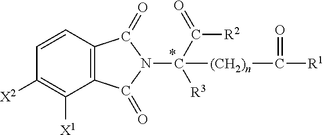

wherein:

one of X¹ and X² is nitro, or NH—Z, and the other of X¹ or X² is hydrogen;

each of R¹ and R², independent of the other, is hydroxy or NH—Z;

R³ is alkyl of one to six carbons, halo, or hydrogen;

Z is hydrogen, phenyl, an acyl of one to six carbons, or an alkyl of one to six carbons; and

n has a value of 0, 1, or 2; and

if —COR² and —(CH₂)ₙCOR¹ are different, the carbon atom designated C constitutes a center of chirality.

Other representative compounds are of formula:

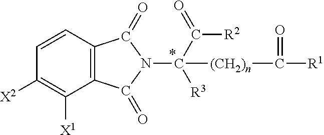

wherein:

one of X¹ and X² is alkyl of one to six carbons;

each of R¹ and R², independent of the other, is hydroxy or NH—Z;

R³ is alkyl of one to six carbons, halo, or hydrogen;

Z is hydrogen, phenyl, an acyl of one to six carbons, or an alkyl of one to six carbons; and

n has a value of 0, 1, or 2; and

if —COR² and —(CH₂)ₙCOR¹ are different, the carbon atom designated C* constitutes a center of chirality.

Still other specific immunomodulatory compounds are isoindoline-1-one and isoindoline-1,3-dione substituted in the 2-position with 2,6-dioxo-3-hydroxypiperidin-5-yl described in U.S. Pat. No. 6,458,810, which is incorporated herein by reference. Representative compounds are of formula:

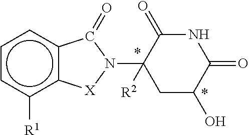

wherein:

the carbon atoms designated constitute centers of chirality;

X is —C(O)— or —CH₂—;

R¹ is alkyl of 1 to 8 carbon atoms or —NHR³;

R² is hydrogen, alkyl of 1 to 8 carbon atoms, or halogen; and

R³ is hydrogen,

alkyl of 1 to 8 carbon atoms, unsubstituted or substituted with alkoxy of 1 to 8 carbon atoms, halo, amino, or alkylamino of 1 to 4 carbon atoms,

cycloalkyl of 3 to 18 carbon atoms,

phenyl, unsubstituted or substituted with alkyl of 1 to 8 carbon atoms, alkoxy of 1 to 8 carbon atoms, halo, amino, or alkylamino of 1 to 4 carbon atoms,

benzyl, unsubstituted or substituted with alkyl of 1 to 8 carbon atoms, alkoxy of 1 to 8 carbon atoms, halo, amino, or alkylamino of 1 to 4 carbon atoms, or —COR⁴ in which

R⁴ is hydrogen,

alkyl of 1 to 8 carbon atoms, unsubstituted or substituted with alkoxy of 1 to 8 carbon atoms, halo, amino, or alkylamino of 1 to 4 carbon atoms,

cycloalkyl of 3 to 18 carbon atoms,

phenyl, unsubstituted or substituted with alkyl of 1 to 8 carbon atoms, alkoxy of 1 to 8 carbon atoms, halo, amino, or alkylamino of 1 to 4 carbon atoms, or

benzyl, unsubstituted or substituted with alkyl of 1 to 8 carbon atoms, alkoxy of 1 to 8 carbon atoms, halo, amino, or alkylamino of 1 to 4 carbon atoms.

Other specific compounds provided herein are of formula:

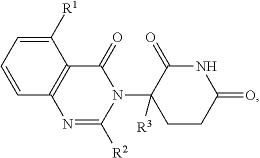

and pharmaceutically acceptable salts, solvates, and stereoisomers thereof, wherein:
R¹ is: hydrogen; halo; —(CH₂)ₙOH; (C₁-C₆)alkyl, optionally substituted with one or more halo; (C₁-C₆)alkoxy, optionally substituted with one or more halo; or

—(CH₂)ₙNHR^{a}, wherein R^{a} is:
hydrogen;
(C₁-C₆)alkyl, optionally substituted with one or more halo;
—(CH₂)ₙ-(6 to 10 membered aryl);
—C(O)—(CH₂)ₙ-(6 to 10 membered aryl) or —C(O)—(CH₂)ₙ-(6 to 10 membered heteroaryl), wherein the aryl or heteroaryl is optionally substituted with one or more of: halo; —SCF₃; (C₁-C₆)alkyl, itself optionally substituted with one or more halo; or (C₁-C₆)alkoxy, itself optionally substituted with one or more halo;
—C(O)—(C₁-C₈)alkyl, wherein the alkyl is optionally substituted with one or more halo;
—C(O)—(CH₂)ₙ—(C₃-C₁₀-cycloalkyl);
—C(O)—(CH₂)ₙ—NR^{b}R^{c}, wherein R^{b} and R^{c} are each independently:
   hydrogen;
   (C₁-C₆)alkyl, optionally substituted with one or more halo;
   (C₁-C₆)alkoxy, optionally substituted with one or more halo; or
   6 to 10 membered aryl, optionally substituted with one or more of: halo; (C₁-C₆)alkyl, itself optionally substituted with one or more halo;

or (C₁-C₆)alkoxy, itself optionally substituted with one or more halo;
—C(O)—(CH₂)ₙ—O—(C₁-C₆)alkyl; or
—C(O)—(CH₂)ₙ—O—(CH₂)ₙ-(6 to 10 membered aryl);
R² is: hydrogen; —(CH₂)ₙOH; phenyl; —O—(C₁-C₆)alkyl; or (C₁-C₆)alkyl, optionally substituted with one or more halo;
R³ is: hydrogen; or (C₁-C₆)alkyl, optionally substituted with one or more halo; and
n is 0, 1, or 2.

Specific examples include, but are not limited to, 3-(5-amino-2-methyl-4-oxo-4H-quinazolin-3-yl)-piperidine-2,6-dione (“Compound A”), which has the following structure:

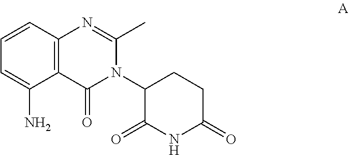 [SMILES: Cc1nc2cccc(N)c2c(=O)n1C1CCC(=O)NC1=O]

or an enantiomer or a mixture of enantiomers thereof; or a pharmaceutically acceptable salt, solvate, hydrate, co-crystal, clathrate, or polymorph thereof.

Compound A can be prepared according to the methods described in the Examples provided herein or as described in U.S. Pat. No. 7,635,700, the disclosure of which is incorporated herein by reference in its entirety. The compound can be also synthesized according to other methods apparent to those of skill in the art based upon the teaching herein. In certain embodiments, Compound A is in a crystalline form described in U.S. Provisional Pat. App. No. 61/451,806, filed Mar. 11, 2011, which is incorporated herein by reference in its entirety. In some embodiments, the hydrochloride salt of Compound A is used in the methods provided herein. Methods of treating, preventing and/or managing cancers and other diseases using Compound A are described in U.S. Provisional Pat. App. No. 61/451,995, filed Mar. 11, 2011, which is incorporated herein by reference in its entirety.

Other specific compounds provided herein are of formula:

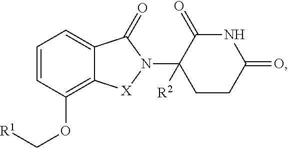

or a pharmaceutically acceptable salt, solvate or stereoisomer thereof, wherein:
X is C═O or CH₂;
R¹ is —Y—R³;
R² is H or (C₁-C₆)alkyl;
Y is: 6 to 10 membered aryl, heteroaryl or heterocycle, each of which may be optionally substituted with one or more halogen; or a bond;
R³ is: —(CH₂)ₙ-aryl, —O—(CH₂)ₙ-aryl or —(CH₂)ₙ—O-aryl, wherein the aryl is optionally substituted with one or more: (C₁-C₆)alkyl, itself optionally substituted with one or more halogen; (C₁-C₆)alkoxy, itself substituted with one or more halogen; oxo; amino; carboxyl; cyano; hydroxyl; halogen; deuterium; 6 to 10 membered aryl or heteroaryl, optionally substituted with one or more
   (C₁-C₆)alkyl, (C₁-C₆)alkoxy or halogen; —CONH₂; or —COO—(C₁-C₆)alkyl, wherein the alkyl may be optionally substituted with one or more halogen;
   —(CH₂)ₙ-heterocycle, —O—(CH₂)-heterocycle or —(CH₂)ₙ—O-heterocycle, wherein the
   heterocycle is optionally substituted with one or more: (C₁-C₆)alkyl, itself optionally substituted with one or more halogen; (C₁-C₆)alkoxy, itself substituted with one or more halogen; oxo; amino; carboxyl; cyano; hydroxyl; halogen; deuterium; 6 to 10 membered aryl or heteroaryl, optionally substituted with one or more (C₁-C₆)alkyl, (C₁-C₆)alkoxy or halogen; —CONH₂; or —COO—(C₁-C₆)alkyl, wherein the alkyl may be optionally substituted with one or more halogen; or —(CH₂)ₙ-heteroaryl, —O—(CH₂)ₙ-heteroaryl or —(CH₂)ₙ—O-heteroaryl, wherein the
   heteroaryl is optionally substituted with one or more: (C₁-C₆)alkyl, itself optionally substituted with one or more halogen; (C₁-C₆)alkoxy, itself substituted with one or more halogen; oxo; amino; carboxyl; cyano; hydroxyl; halogen; deuterium; 6 to 10 membered aryl or heteroaryl, optionally substituted with one or more (C₁-C₆)alkyl, (C₁-C₆)alkoxy or halogen; —CONH₂; or —COO—(C₁-C₆)alkyl, wherein the alkyl may be optionally substituted with one or more halogen; and
      n is 0, 1, 2 or 3.

Specific examples include, but are not limited to, 3-(4-((4-(morpholinomethyl)benzyl)oxy)-1-oxoisoindolin-2-yl)piperidine-2,6-dione. In one embodiment, provided herein is the (S) stereoisomer of 3-(4-((4-(morpholinomethyl)benzyl)oxy)-1-oxoisoindolin-2-yl)piperidine-2,6-dione (“Compound B”) e.g., for use in the methods described herein. Racemic 3-(4-((4-(morpholinomethyl)benzyl)oxy)-1-oxoisoindolin-2-yl)piperidine-2,6-dione and methods of preparing the same have been reported in U.S. Patent Publication No. 2011/0196150, which is incorporated herein by reference in its entirety. Compound B has the following structure:

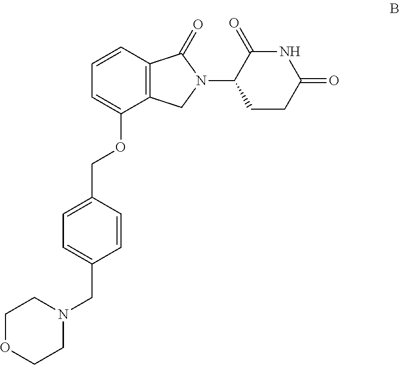 [SMILES: O=C1CC[C@H](N2Cc3c(OCc4ccc(CN5CCOCC5)cc4)cccc3C2=O)C(=O)N1]

All of the compounds described can either be commercially purchased or prepared according to the methods described in the patents or patent publications disclosed herein. Further, optically pure compounds can be asymmetrically synthesized or resolved using known resolving agents or chiral columns as well as other standard synthetic organic chemistry techniques. Additional information on immunomodulatory compounds, their preparation, and use can be found, for example, in U.S. Patent Application Publication Nos. US20060188475, US20060205787, and US20070049618, each of which is incorporated by reference herein in its entirety.

The immunomodulatory therapies may be small organic molecules having a molecular weight less than about 1,000 g/mol, and are not proteins, peptides, oligonucleotides, oligosaccharides or other macromolecules.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

### 5.7 Combination Therapy

One or more additional therapies, such as additional active ingredients or agents, that can be used in combination with an immunomodulatory therapy, such as described in Section 5.6, supra. In a specific embodiment, one or more additional active ingredients or agents can be used in the methods and compositions provided herein with an immunomodulatory therapy. The one or more additional therapies can be administered prior to, concurrently with, or subsequent to the administration of an immunomodulatory therapy. Administration of an immunomodulatory therapy and an additional active agent to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the cancer being treated. Preferred routes of administration for the additional active agents or ingredients of the invention are known to those of ordinary skill in the art. See, e.g., Physicians' Desk Reference.

In certain embodiments, an immunomodulatory therapy and an additional active agent are cyclically administered to a patient with a hematological cancer (e.g., DLBCL). Cycling therapy involves the administration of an active agent for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

The additional active agents administered in combination with an immunomodulatory therapy can be large molecules (e.g., proteins) or small molecules (e.g., synthetic inorganic, organometallic, or organic molecules). In certain embodiments, the additional active agent is another immunomodulatory therapy. In other embodiments, the additional active agent is not an immunomodulatory therapy. Examples of large molecule active agents include, but are not limited to, hematopoietic growth factors, cytokines, and monoclonal and polyclonal antibodies. In certain embodiments, large molecule active agents are biological molecules, such as naturally occurring or artificially made proteins. Proteins that are useful include proteins that stimulate the survival and/or proliferation of hematopoietic precursor cells and immunologically active poietic cells in vitro or in vivo. Others stimulate the division and differentiation of committed erythroid progenitors in cells in vitro or in vivo. Particular proteins include, but are not limited to: interleukins, such as IL-2 (including recombinant IL-I1 (“rIL2”) and canarypox IL-2), IL-10, IL-12, and IL-18; interferons, such as interferon alfa-2a, interferon alfa-2b, interferon alfa-n1, interferon alfa-n3, interferon beta-I a, and interferon gamma-I b; GM-CF and GM-CSF; and EPO.

Particular proteins that can be used in the methods and compositions of the disclosure include, but are not limited to: filgrastim, which is sold in the United States under the trade name NEUPOGEN® (Amgen, Thousand Oaks, Calif.); sargramostim, which is sold in the United States under the trade name LEUKINE® (Immunex, Seattle, Wash.); and recombinant EPO, which is sold in the United States under the trade name EPGEN® (Amgen, Thousand Oaks, Calif.).

Inhibitors of ActRII receptors or activin-ActRII inhibitors may be used in the methods and compositions provided herein. ActRII receptors include ActRIIA inhibitors and ActRIIB inhibitors. Inhibitors of ActRII receptors can be polypeptides comprising activin-binding domains of ActRII. In certain embodiments, the activin-binding domain comprising polypeptides are linked to an Fc portion of an antibody (i.e., a conjugate comprising an activin-binding domain comprising polypeptide of an ActRII receptor and an Fc portion of an antibody is generated). In certain embodiments, the activin-binding domain is linked to an Fc portion of an antibody via a linker, e.g., a peptide linker. Examples of such non-antibody proteins selected for activin or ActRIIA binding and methods for design and selection of the same are found in WO/2002/088171, WO/2006/055689, WO/2002/032925, WO/2005/037989, US 2003/0133939, and US 2005/0238646, each of which is incorporated herein by reference in its entirety.

Recombinant and mutated forms of GM-CSF can be prepared as described in U.S. Pat. Nos. 5,391,485; 5,393,870; and 5,229,496; the disclosure of each of which is incorporated herein by reference in its entirety. Recombinant and mutated forms of G-CSF can be prepared as described in U.S. Pat. Nos. 4,810,643; 4,999,291; 5,528,823; and 5,580,755; the disclosure of each of which is incorporated herein by reference in its entirety.

This disclosure encompasses the use of native, naturally occurring, and recombinant proteins. The disclosure further encompasses mutants and derivatives (e.g., modified forms) of naturally occurring proteins that exhibit, in vivo, at least some of the pharmacological activity of the proteins upon which they are based. Examples of mutants include, but are not limited to, proteins that have one or more amino acid residues that differ from the corresponding residues in the naturally occurring forms of the proteins. Also encompassed by the term “mutants” are proteins that lack carbohydrate moieties normally present in their naturally occurring forms (e.g., nonglycosylated forms). Examples of derivatives include, but are not limited to, pegylated derivatives and fusion proteins, such as proteins formed by fusing IgG1 or IgG3 to the protein or active portion of the protein of interest. See, e.g., Penichet, M. L. and Morrison, S. L., J. Immunol. Methods 248:91-101 (2001).

Antibodies that can be used in combination with an immunomodulatory therapy include monoclonal and polyclonal antibodies. Examples of antibodies include, but are not limited to, trastuzumab (HERCEPTIN®), rituximab (RITUXAN®), bevacizumab (AVASTIN®), pertuzumab (OMNITARG™), tositumomab (BEXXAR®), edrecolomab (PANOREX®), panitumumab and G250. An immunomodulatory therapy provided herein can also be combined with or used in combination with anti-TNF-alpha antibodies.

Large molecule active agents may be administered in the form of anti-cancer vaccines.

For example, vaccines that secrete, or cause the secretion of, cytokines such as IL-2, SCF, CXC14 (platelet factor 4), G-CSF, and GM-CSF can be used in the methods, pharmaceutical compositions, and kits of the disclosure. See, e.g., Emens, L. A., et al., Curr. Opinion Mol. Ther. 3(1):77-84 (2001).

Additional active agents that are small molecules can also be used to alleviate adverse effects associated with the administration of an immunomodulatory therapy. However, like some large molecules, many are believed to be capable of providing a synergistic effect when administered with (e.g., before, after or simultaneously) the immunomodulatory therapy. Examples of small molecule additional active agents include, but are not limited to, anti-cancer agents, antibiotics, immunosuppressive agents, and steroids.

Examples of anti-cancer agents include, but are not limited to: abraxane; ace-11; acivicin; aclarubicin; acodazole hydrochloride; acronine; adozelesin; aldesleukin; altretamine; ambomycin; ametantrone acetate; amrubicin; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer, carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; herceptin; hydroxyurea; idarubicin hydrochloride; ifosfamide; ilmofosine; iproplatin; irinotecan; irinotecan hydrochloride; lanreotide acetate; lapatinib; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper, mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porflmer sodium; porfiromycin; prednimustine; procarbazine hydrochloride; puromycin; puromycin hydrochloride; pyrazofurin; riboprine; romidepsin; safingol; safingol hydrochloride; semustine; simtrazene; sparfosate sodium; sparsomycin; spirogermanium hydrochloride; spiromustine; spiroplatin; stem cell treatments such as PDA-001; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; b-FGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor, carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., GLEEVEC®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (GENASENSE®); O.sup.6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor, platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor, protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

Specific additional active agents include, but are not limited to, oblimersen (GENASENSE®), remicade, docetaxel, celecoxib, melphalan, dexamethasone (DECADRON®), steroids, gemcitabine, cisplatinum, temozolomide, etoposide, cyclophosphamide, temodar, carboplatin, procarbazine, gliadel, tamoxifen, topotecan, methotrexate, ARISA®, taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin (DOXIL®), paclitaxel, ganciclovir, adriamycin, estramustine sodium phosphate) (EMCYT.degree.), sulindac, and etoposide.

### 5.8 Biological Samples

In certain embodiments, the various methods provided herein use samples (e.g., biological samples) from subjects or individuals (e.g., patients). In a specific embodiment, the subject is a patient with a hematological cancer, such as multiple myeloma, leukemia or a lymphoma. The subject can be a mammal, for example, a human. The subject can be male or female, and can be an adult, child or infant. Samples can be analyzed at a time during an active phase of a disease or disorder, or when a disease or disorder is inactive. In a specific embodiment, a sample is obtained from a subject prior, concurrently with and/or subsequent to administration of an immunomodulatory therapy. In certain embodiments, more than one sample from a subject can be obtained.

In certain embodiments, the sample used in the methods provided herein comprises body fluids from a subject. Non-limiting examples of body fluids include blood (e.g., peripheral whole blood, peripheral blood), blood plasma, amniotic fluid, aqueous humor, bile, cerumen, cowper's fluid, pre-ejaculatory fluid, chyle, chyme, female ejaculate, interstitial fluid, lymph, menses, breast milk, mucus, pleural fluid, pus, saliva, sebum, semen, serum, sweat, tears, urine, vaginal lubrication, vomit, water, feces, internal body fluids, including cerebrospinal fluid surrounding the brain and the spinal cord, synovial fluid surrounding bone joints, intracellular fluid is the fluid inside cells, and vitreous humour the fluids in the eyeball. In some embodiments, the sample is a blood sample. The blood sample can be obtained using conventional techniques as described in, e.g. Innis et al, editors, PCR Protocols (Academic Press, 1990). White blood cells can be separated from blood samples using convention techniques or commercially available kits, e.g. RosetteSep™ kit (Stein Cell Technologies, Vancouver, Canada). Sub-populations of white blood cells, e.g. mononuclear cells, B cells, T cells, monocytes, granulocytes or lymphocytes, can be further isolated using conventional techniques, e.g. magnetically activated cell sorting (MACS) (Miltenyi Biotec. Auburn, Calif.) or fluorescently activated cell sorting (FACS) (Becton Dickinson, San Jose, Calif.).

In one embodiment, the blood sample is from about 0.1 mL to about 10.0 mL, from about 0.2 mL to about 7 mL, from about 0.3 mL to about 5 mL, from about 0.4 mL to about 3.5 mL, or from about 0.5 mL to about 3 mL. In another embodiment, the blood sample is about 0.3, 0.4, 0.5, 0.6, 0.7, 0.8, 0.9, 1.0, 1.5, 2.0, 2.5, 3.0, 3.5, 4.0, 4.5, 5.0, 6.0, 7.0, 8.0, 9.0 or 10.0 mL.

In some embodiments, the sample used in the present methods comprises a biopsy (e.g., a tumor biopsy). The biopsy can be from any organ or tissue, for example, skin, liver, lung, heart, colon, kidney, bone marrow, teeth, lymph node, hair, spleen, brain, breast, or other organs. Any biopsy technique known by those skilled in the art can be used for isolating a sample from a subject, for instance, open biopsy, close biopsy, core biopsy, incisional biopsy, excisional biopsy, or fine needle aspiration biopsy.

In one embodiment, the sample used in the methods provided herein is obtained from the subject prior to the subject receiving a treatment for the hematological cancer. In another embodiment, the sample is obtained from the subject during the subject receiving a treatment for the hematological cancer. In another embodiment, the sample is obtained from the subject after the subject receiving a treatment for the hematological cancer. In various embodiments, the treatment comprises administering an immunomodulatory therapy (e.g., a compound provided in Section 5.6) to the subject.

In certain embodiments, the sample used in the methods provided herein comprises a plurality of cells. Such cells can include any type of cells, e.g., stem cells, blood cells (e.g., peripheral blood mononuclear cells), lymphocytes, B cells, T cells, monocytes, granulocytes, immune cells, or tumor or cancer cells. The tumor or cancer cells or a tumor tissue, such as a tumor biopsy or a tumor explants. T cells (T lymphocytes) include, for example, helper T cells (effector T cells or Th cells), cytotoxic T cells (CTLs), memory T cells, and regulatory T cells. In one embodiment, the cells used in the methods provided herein are CD3⁺ T cells, e.g., as detected by flow cytometry. The number of T cells used in the methods can range from a single cell to about 10⁹ cells. B cells (B lymphocytes) include, for example, plasma B cells, dendritic cells, memory B cells, B1 cells, B2 cells, marginal-zone B cells, and follicular B cells. B cells can express immunoglobulins (antibodies, B cell receptor).

Specific cell populations can be obtained using a combination of commercially available antibodies (e.g., Quest Diagnostic (San Juan Capistrano, Calif.); Dako (Denmark)).

In some embodiments, the cancer is a hematological cancer. In one embodiment, the blood cancer is multiple myeloma. In another embodiment, the blood cancer is chronic lymphocytic leukemia (CLL). In another embodiment, the blood cancer is DLBCL. In another embodiment, the blood cancer is myelodysplastic syndrome, an acute leukemia, e.g., acute T cell leukemia, acute myelogenous leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), or acute biphenotypic leukemia; a chronic leukemia, e.g., chronic myeloid lymphoma, chronic myelogenous leukemia (CML), chronic monocytic leukemia, Small lymphocytic lymphoma, or B-cell prolymphocytic leukemia; hairy cell lymphoma; T-cell prolymphocytic leukemia, or a lymphoma, e.g, histiocytic lymphoma, lymphoplasmacytic lymphoma (e.g., Waldenström macroglobulinemia), splenic marginal zone lymphoma, plasma cell neoplasm (e.g., plasma cell myeloma, plasmacytoma, a monoclonal immunoglobulin deposition disease, or a heavy chain disease), extranodal marginal zone B cell lymphoma (MALT lymphoma), nodal marginal zone B cell lymphoma (NMZL), follicular lymphoma, mantle cell lymphoma, diffuse large B cell lymphoma, mediastinal (thymic) large B cell lymphoma, intravascular large B cell lymphoma, primary effusion lymphoma, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides (Sezary syndrome), a primary cutaneous CD30-positive T cell lymphoproliferative disorder (e.g., primary cutaneous anaplastic large cell lymphoma or lymphomatoid papulosis), angioimmunoblastic T cell lymphoma, peripheral T cell lymphoma, unspecified, anaplastic large cell lymphoma, a Hodgkin's lymphoma or a nodular lymphocyte-predominant Hodgkin's lymphoma.

In certain embodiments, the sample used in the methods provided herein is from a diseased tissue, e.g., from an individual having a hematological cancer. In certain embodiments, the number of cells used in the methods provided herein can range from a single cell to about 10⁹ cells. In some embodiments, the number of cells used in the methods provided herein is about 1×10⁴, 5×10⁴, 1×10⁵, 5×10⁵, 1×10⁶, 5×10⁶, 1×10⁷, 5×10⁷, 1×10⁸, or 5×10⁸.

The number and type of cells collected from a subject can be monitored, for example, by measuring changes in morphology and cell surface markers using standard cell detection techniques such as flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, and/or by measuring changes in gene expression using techniques well known in the art, such as PCR and gene expression profiling. These techniques can be used, too, to identify cells that are positive for one or more particular markers. Fluorescence activated cell sorting (FACS) is a well-known method for separating particles, including cells, based on the fluorescent properties of the particles (Kamarch, 1987, Methods Enzymol, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In certain embodiments, subsets of cells are used in the methods provided herein. Methods to sort and isolate specific populations of cells are well-known in the art and can be based on cell size, morphology, or intracellular or extracellular markers. Such methods include, but are not limited to, flow cytometry, flow sorting, FACS, bead based separation such as magnetic cell sorting, size-based separation (e.g., a sieve, an array of obstacles, or a filter), sorting in a microfluidics device, antibody-based separation, sedimentation, affinity adsorption, affinity extraction, density gradient centrifugation, laser capture microdissection, etc.

### 5.9 Methods for Detecting RNA Expression

Several methods of detecting or quantitating mRNA levels are known in the art. Exemplary methods include but are not limited to northern blots, ribonuclease protection assays, PCR-based methods, and the like. The mRNA sequence can be used to prepare a probe that is at least partially complementary. The probe can then be used to detect the mRNA sequence in a sample, using any suitable assay, such as PCR-based methods. Northern blotting, a dipstick assay, and the like.

In other embodiments, a nucleic acid assay for testing for immunomodulatory activity in a biological sample can be prepared. An assay typically contains a solid support and at least one nucleic acid contacting the support, where the nucleic acid corresponds to at least a portion of an mRNA encoded by a gene listed in Table 1, 2, 3 or 4. The assay can also have a means for detecting the altered expression of the mRNA in the sample.

The assay method can be varied depending on the type of mRNA information desired. Exemplary methods include but are not limited to Northern blots and PCR-based methods (e.g., qRT-PCR). Methods such as qRT-PCR can also accurately quantitate the amount of the mRNA in a sample.

Any suitable assay platform can be used to determine the presence of the mRNA in a sample. For example, an assay may be in the form of a dipstick, a membrane, a chip, a disk, a test strip, a filter, a microsphere, a slide, a multiwell plate, or an optical fiber. An assay system may have a solid support on which a nucleic acid corresponding to the mRNA is attached. The solid support may comprise, for example, a plastic, silicon, a metal, a resin, glass, a membrane, a particle, a precipitate, a gel, a polymer, a sheet, a sphere, a polysaccharide, a capillary, a film a plate, or a slide. The assay components can be prepared and packaged together as a kit for detecting an mRNA.

The nucleic acid can be labeled, if desired, to make a population of labeled mRNAs. In general, a sample can be labeled using methods that are well known in the art (e.g., using DNA ligase, terminal transferase, or by labeling the RNA backbone, etc.; see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). In some embodiments, the sample is labeled with fluorescent label. Exemplary fluorescent dyes include but are not limited to xanthene dyes, fluorescein dyes, rhodamine dyes, fluorescein isothiocyanate (FITC), 6 carboxyfluorescein (FAM), 6 carboxy-2′,4′,7′,4,7-hexachlorofluorescein (HEX), 6 carboxy 4′, 5′ dichloro 2′, 7′ dimethoxyfluorescein (JOE or J), N,N,N′,N′ tetramethyl 6 carboxyrhodamine (TAMRA or T), 6 carboxy X rhodamine (ROX or R), 5 carboxyrhodamine 6G (R6G5 or G5), 6 carboxyrhodamine 6G (R6G6 or G6), and rhodamine 110; cyanine dyes, e.g. Cy3, Cy5 and Cy7 dyes; Alexa dyes, e.g. Alexa-fluor-555; coumarin, Diethylaminocoumarin, umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, BODIPY dyes, quinoline dyes, Pyrene, Fluorescein Chlorotriazinyl, R110, Eosin, JOE, R6G, Tetramethylrhodamine, Lissamine, ROX, Napthofluorescein, and the like.

In some embodiments, the mRNA sequences comprise at least one mRNA selected from the mRNAs encoded by the genes listed in Table 3, or a fragment thereof. The nucleic acids may be present in specific, addressable locations on a solid support; each corresponding to at least a portion of mRNA sequences that are differentially expressed upon treatment of an immunomodulatory compound in a cell or a patient.

A typical mRNA assay method can contain the steps of 1) obtaining surface-bound subject probes; 2) hybridization of a population of mRNAs to the surface-bound probes under conditions sufficient to provide for specific binding (3) post-hybridization washes to remove nucleic acids not bound in the hybridization; and (4) detection of the hybridized mRNAs. The reagents used in each of these steps and their conditions for use may vary depending on the particular application.

Hybridization can be carried out under suitable hybridization conditions, which may vary in stringency as desired. Typical conditions are sufficient to produce probe/target complexes on a solid surface between complementary binding members, i.e., between surface-bound subject probes and complementary mRNAs in a sample. In certain embodiments, stringent hybridization conditions may be employed.

Hybridization is typically performed under stringent hybridization conditions. Standard hybridization techniques (e.g. under conditions sufficient to provide for specific binding of target mRNAs in the sample to the probes) are described in Kallioniemi et al., Science 258:818-821 (1992) and WO 93/18186. Several guides to general techniques are available, e.g., Tijssen, Hybridization with Nucleic Acid Probes, Parts I and II (Elsevier, Amsterdam 1993). For descriptions of techniques suitable for in situ hybridizations, see Gall et al. Meth. Enzymol., 21:470-480 (1981); and Angerer et al. in Genetic Engineering: Principles and Methods (Setlow and Hollaender, Eds.) Vol 7, pgs 43-65 (Plenum Press, New York 1985). Selection of appropriate conditions, including temperature, salt concentration, polynucleotide concentration, hybridization time, stringency of washing conditions, and the like will depend on experimental design, including source of sample, identity of capture agents, degree of complementarity expected, etc., and may be determined as a matter of routine experimentation for those of ordinary skill in the art.

Those of ordinary skill will readily recognize that alternative but comparable hybridization and wash conditions can be utilized to provide conditions of similar stringency.

After the mRNA hybridization procedure, the surface bound polynucleotides are typically washed to remove unbound nucleic acids. Washing may be performed using any convenient washing protocol, where the washing conditions are typically stringent, as described above. The hybridization of the target mRNAs to the probes is then detected using standard techniques.

Other methods, such as PCR-based methods, can also be used to follow the expression of the genes in Table 1, 2, or 3. Examples of PCR methods can be found in the literature. Examples of PCR assays can be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

In some embodiments, Real-Time Reverse Transcription-PCR (qRT-PCR) can be used for both the detection and quantification of RNA targets (Bustin, et al., 2005, Clin. Sci., 109:365-379). Quantitative results obtained by qRT-PCR are generally more informative than qualitative data. Thus, in some embodiments, qRT-PCR-based assays can be useful to measure mRNA levels during cell-based assays. The qRT-PCR method is also useful to monitor patient therapy. Examples of qRT-PCR-based methods can be found, for example, in U.S. Pat. No. 7,101,663, which is incorporated by reference herein in its entirety.

In contrast to regular reverse transcriptase-PCR and analysis by agarose gels, real-time PCR gives quantitative results. An additional advantage of real-time PCR is the relative ease and convenience of use. Instruments for real-time PCR, such as the Applied Biosystems 7500, are available commercially, as are the reagents, such as TaqMan Sequence Detection chemistry. For example, TaqMan® Gene Expression Assays can be used, following the manufacturer's instructions. These kits are pre-formulated gene expression assays for rapid, reliable detection and quantification of human, mouse and rat mRNA transcripts. An exemplary PCR program, for example, is 50° C. for 2 minutes, 95° C. for 10 minutes, 40 cycles of 95° C. for 15 seconds, then 60° C. for 1 minute.

To determine the cycle number at which the fluorescence signal associated with a particular amplicon accumulation crosses the threshold (referred to as the CT), the data can be analyzed, for example, using a 7500 Real-Time PCR System Sequence Detection software v1.3 using the comparative CT relative quantification calculation method. Using this method, the output is expressed as a fold-change of expression levels. In some embodiments, the threshold level can be selected to be automatically determined by the software. In some embodiments, the threshold level is set to be above the baseline but sufficiently low to be within the exponential growth region of an amplification curve.

### 5.10 Methods for Detecting Protein Expression

Several protein detection and quantitation methods can be used to measure the level of proteins. Any suitable protein quantitation method can be used. In some embodiments, antibody-based methods are used. Exemplary methods that can be used include but are not limited to immunoblotting (western blot), enzyme-linked immunosorbent assay (ELISA), immunohistochemistry, flow cytometry, cytometric bead array, mass spectroscopy, and the like. Several types of ELISA are commonly used, including direct ELISA, indirect ELISA, and sandwich ELISA.

### 5.11 Kits

In one aspect, provided herein are pharmaceutical or assay kits comprising an immunomodulatory therapy or a pharmaceutical composition thereof, in one or more containers, and instructions for use. In certain embodiments, the kits useful for predicting the likelihood of an effective patient tumor response. In further embodiments, the immunomodulatory therapy, in a container, is accompanied by an apparatus or apparati necessary for administering the compound or composition thereof to a subject.

In certain embodiments, a kit comprises an immunomodulatory therapy or pharmaceutical composition thereof, in a container, and a reagent or reagents necessary for carrying out an assay(s) described herein, in one or more other containers. In certain embodiments, the kit comprises a solid support, and a means for detecting the RNA or protein expression of at least one biomarker (e.g., a differentially expressed gene identified in Table 1, 2, 3, or 4) in a biological sample. Such a kit may employ, for example, a dipstick, a membrane, a chip, a disk, a test strip, a filter, a microsphere, a slide, a multiwell plate, or an optical fiber. The solid support of the kit can be, for example, a plastic, silicon, a metal, a resin, glass, a membrane, a particle, a precipitate, a gel, a polymer, a sheet, a sphere, a polysaccharide, a capillary, a film, a plate, or a slide.

In a specific embodiment, the pharmaceutical or assay kit comprises, in a container, an immunomodulatory therapy or a pharmaceutical composition thereof, and further comprises, in one or more containers, components for isolating RNA. In another specific embodiment, the pharmaceutical or assay kit comprises, in a container, an immunomodulatory therapy or a pharmaceutical composition, and further comprises, in one or more containers, components for conducting RT-PCR, RT-qPCR, deep sequencing or a microarray. In some embodiments, the kit comprises a solid support, nucleic acids contacting the support, where the nucleic acids are complementary to at least 20, 50, 100, 200, 350, or more bases of mRNA, and a means for detecting the expression of the mRNA in a biological sample.

In another specific embodiment, the pharmaceutical or assay kit comprises, in a container, an immunomodulatory therapy or a pharmaceutical composition thereof, and further comprises, in one or more containers, components for isolating protein In another specific embodiment, the pharmaceutical or assay kit comprises, in a container, an immunomodulatory therapy or a pharmaceutical composition, and further comprises, in one or more containers, components for conducting flow cytometry or an ELISA.

In another aspect, provided herein are kits for measuring biomarkers providing the materials necessary to measure the abundance of one or more of the gene products of the genes or a subset of genes (e.g., one, two, three, four, five or more genes) in Table 1, 2, 3 or 4, or any combination thereof. Such kits may comprise materials and reagents required for measuring RNA or protein. In some embodiments, such kits include microarrays, wherein the microarray is comprised of oligonucleotides and/or DNA and/or RNA fragments which hybridize to one or more of the products of one or more of the genes or a subset of genes in Table 1, 2, 3 or 4, or any combination thereof. In some embodiments, such kits may include primers for PCR of either the RNA product or the cDNA copy of the RNA product of the genes or subset of genes, or both. In some embodiments, such kits may include primers for PCR as well as probes for Quantitative PCR. In some embodiments, such kits may include multiple primers and multiple probes wherein some of said probes have different fluorophores so as to permit multiplexing of multiple products of a gene product or multiple gene products. In some embodiments, such kits may further include materials and reagents for creating cDNA from RNA. In some embodiments, such kits may include antibodies specific for the protein products of a gene or subset of genes in Table 1, 2, 3, or 4, or any combination thereof. Such kits may additionally comprise materials and reagents for isolating RNA and/or proteins from a biological sample. In addition such kits may include materials and reagents for synthesizing cDNA from RNA isolated from a biological sample. In some embodiments, such kits may include, a computer program product embedded on computer readable media for predicting whether a patient is clinically sensitive to an immunomodulatory therapy. In some embodiments, the kits may include a computer program product embedded on a computer readable media along with instructions.

In some embodiments, kits for measuring the expression of one or more nucleic acid sequences of a gene or a subset of genes in Table 1, 2, 3 or 4 or a combination thereof. In a specific embodiment, such kits measure the expression of one or more nucleic acid sequences associated with a gene or a subset of genes in Table 1, 2, 3 or 4, or a combination thereof. In accordance with this embodiment, the kits may comprise materials and reagents that are necessary for measuring the expression of particular nucleic acid sequence products of genes or a subset of genes in Table 1, 2, 3 or 4, or a combination thereof. For example, a microarray or RT-PCR kit may be produced for a specific condition and contain only those reagents and materials necessary for measuring the levels of specific RNA transcript products of the genes or a subset of genes in Table 1, 2, 3 or 4, or a combination thereof to predict whether a hematological cancer in a patient is clinically sensitive to an immunomodulatory therapy. Alternatively, in some embodiments, the kits can comprise materials and reagents that are not limited to those required to measure the expression of particular nucleic acid sequences of any particular gene in Table 1, 2, 3, or 4, or a combination thereof. For example, in certain embodiments, the kits comprise materials and reagents necessary for measuring the levels of expression of 1, 2, 3, 4, 5, 6, 7, 8, 9, 10, 15, 20, 25, 30, 35, 40, 45, 50 or more of the genes in Table 1, 2, 3 or 4, in addition to reagents and materials necessary for measuring the levels of the expression of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50 or more genes other than those in Table 1, 2, 3 or 4. In other embodiments, the kits contain reagents and materials necessary for measuring the levels of expression of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50 or more of the genes in Table 1, 2, 3 or 4, and 1, 2, 3, 4, 5, 10, 15, 20, 25, 30, 35, 40, 45, 50, 55, 60, 65, 70, 75, 80, 85, 90, 95, 100, 125, 150, 175, 200, 225, 250, 300, 350, 400, 450, or more genes that are genes not in Table 1, 2, 3 or 4, or 1-10, 1-100, 1-150, 1-200, 1-300, 1-400, 1-500, 1-1000, 25-100, 25-200, 25-300, 25-400, 25-500, 25-1000, 100-150, 100-200, 100-300, 100-400, 100-500, 100-1000 or 500-1000 genes that are genes not in Table 1, 2, 3 or 4.

For nucleic acid microarray kits, the kits generally comprise probes attached to a solid support surface. In one such embodiment, probes can be either oligonucleotides or longer length probes including probes ranging from 150 nucleotides in length to 800 nucleotides in length. The probes may be labeled with a detectable label. In a specific embodiment, the probes are specific for one or more of the gene products in Table 1, 2, 3 or 4. The microarray kits may comprise instructions for performing the assay and methods for interpreting and analyzing the data resulting from the performance of the assay. In a specific embodiment, the kits comprise instructions for predicting whether a hematological cancer in a patient is clinically sensitive to an immunomodulatory therapy. The kits may also comprise hybridization reagents and/or reagents necessary for detecting a signal produced when a probe hybridizes to a target nucleic acid sequence. Generally, the materials and reagents for the microarray kits are in one or more containers. Each component of the kit is generally in its own a suitable container.

In certain embodiments, a nucleic acid microarray kit comprises materials and reagents necessary for measuring the levels of expression of 1, 2, 3, 4, 5, 6, 7, 8, 9, 10, 15, 20, 25, 30, 35, 40, 45, 50 or more of the genes identified in Table 1, 2, 3 or 4, or a combination thereof, in addition to reagents and materials necessary for measuring the levels of the expression of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50 or more genes other than those in Tables 1, 2, 3 or 4. In other embodiments, a nucleic acid microarray kit contains reagents and materials necessary for measuring the levels of expression of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50 or more of the genes in Table 1, 2, 3 or 4, or any combination thereof, and 1, 2, 3, 4, 5, 10, 15, 20, 25, 30, 35, 40, 45, 50, 55, 60, 65, 70, 75, 80, 85, 90, 95, 100, 125, 150, 175, 200, 225, 250, 300, 350, 400, 450, or more genes that are not in Table 1, 2, 3 or 4, or 1-10, 1-100, 1-150, 1-200, 1-300, 1-400, 1-500, 1-1000, 25-100, 25-200, 25-300, 25-400, 25-500, 25-1000, 100-150, 100-200, 100-300, 100-400, 100-500, 100-1000 or 500-1000 genes that are not in Table 1, 2, 3 or 4.

For Quantitative PCR, the kits generally comprise pre-selected primers specific for particular nucleic acid sequences. The Quantitative PCR kits may also comprise enzymes suitable for amplifying nucleic acids (e.g., polymerases such as Taq), and deoxynucleotides and buffers needed for the reaction mixture for amplification. The Quantitative PCR kits may also comprise probes specific for the nucleic acid sequences associated with or indicative of a condition. The probes may or may not be labeled with a fluorophore. The probes may or may not be labeled with a quencher molecule. In some embodiments the Quantitative PCR kits also comprise components suitable for reverse-transcribing RNA including enzymes (e.g., reverse transcriptases such as AMV, MMLV and the like) and primers for reverse transcription along with deoxynucleotides and buffers needed for the reverse transcription reaction. Each component of the quantitative PCR kit is generally in its own suitable container. Thus, these kits generally comprise distinct containers suitable for each individual reagent, enzyme, primer and probe. Further, the quantitative PCR kits may comprise instructions for performing the assay and methods for interpreting and analyzing the data resulting from the performance of the assay. In a specific embodiment, the kits contain instructions for predicting whether a hematological cancer in a patient is clinically sensitive to an immunomodulatory therapy.

For antibody based kits, the kit can comprise, for example: (1) a first antibody (which may or may not be attached to a solid support) which binds to a peptide, polypeptide or protein of interest; and, optionally, (2) a second, different antibody which binds to either the peptide, polypeptide or protein, or the first antibody and is conjugated to a detectable label (e.g., a fluorescent label, radioactive isotope or enzyme). In a specific embodiment, the peptide, polypeptide or protein of interest is associated with or indicative of a condition (e.g., a disease). The antibody-based kits may also comprise beads for conducting an immunoprecipitation. Each component of the antibody-based kits is generally in its own suitable container. Thus, these kits generally comprise distinct containers suitable for each antibody. Further, the antibody-based kits may comprise instructions for performing the assay and methods for interpreting and analyzing the data resulting from the performance of the assay. In a specific embodiment, the kits contain instructions for predicting whether a hematological cancer in a patient is clinically sensitive to an immunomodulatory therapy.

### 6. EXAMPLES

Certain embodiments of the invention are illustrated by the following non-limiting examples.

Gene expression differences between the baseline transcriptional profiles of refractory or relapsed diffuse large B-Cell lymphoma (DLBCL) patients who display response subsequent to lenalidomide treatment and those of patients who do not respond were investigated. A clinical trial was formed with four arms, each arm containing 25 patients. One arm contained patients classified as presenting germinal center B-cell-like DLBCL subtypes receiving lenalidomide, a second arm contained patients classified as presenting germinal center B-cell-like DLBCL subtypes receiving another therapy selected by the investigator, a third arm contained patients classified as presenting activated B-cell-like DLBCL subtypes receiving lenalidomide, and a fourth arm contained patients classified as presenting activated B-cell-like DLBCL subtypes receiving another therapy selected by the investigator. The patients in the four arms of the clinical trial received treatment until disease progression.

For purposes of this exploratory analysis, a subset of the clinical data associated to each patient comprised the therapy arm, Revlimid (REV) or control drug (CON), and their corresponding response to the drug in both categorical variable {complete response (CR), partial response (PR), establish disease (SD), progression disease (PD) and Death} and continuous variables as progression free survival (PFS) and overall (OS), unit weeks. It also includes the predicted DLBCL sub-type (ABC/GCB) and other demographic data.

Samples from patient biopsies taken prior to receiving therapy were hybridized to Affymetrix HG-U 133 Plus 2.0 GeneChip™ microarrays (www.afymetrix.com/) at the Molecular Characterization & Clinical Assay Development Laboratory, SAIC Frederick National Laboratory for Cancer Research, SAIC-Frederick, Frederick, Md. Biopsy samples were flash-frozen at screen (FF), archived having been formalin-fixed and paraffin embedded (FFPE archive), or FFPE treated at screen. All patients are associated with at least one gene expression profile obtained from one of the three sample types, some are associated with more than one profile. The results described in this Section 6 were obtained from analysis of only those profiles relating to FF samples and which passed QC.

Microarray Data QC

Raw Affymetrix image (.cel) files were imported into the R statistical programming environment v3.0.0 (r-project.org) using functionality of the Affy package of the related Bioconductor suite of open-source bioinformatics software (bioconductor.org). Transcriptional profile QC was performed using the NUSE algorithm, implemented in the Bioconductor package arrayQualityMetrics (Kauffmann et al., 2009), applied to a log 2 transformation of raw signal.

The RMA (Irizarry et al., 2003) algorithm was applied to background-correct, quantile normalize and summarize profiles that passed QC. Annotation of probe-sets to genes was performed using the R packages annotate (Gentleman, 2013) and genefilter (Gentleman et al., 2013) selecting only one probeset per gene (Entrez Gene ID) and choosing the most variable across profiles according to inter-quartile range in cases wherein multiple probe-sets map to a single gene.

Clustering & Visualization

Data visualization via clustering and heatmap graphics was implemented in the R statistical programming environment. Either Euclidean distance and correlation (1—Pearson correlation) were used to represent inter-profile dissimilarity and (1—pearson correlation) to represent dissimilarity between genes across profiles. In both cases, hierarchical clustering using Ward's algorithm was applied. Comparative gene expression heatmaps were implemented using gplots (Warnes et al., 2013) and heatmap.plus (Day, 2012) packages from the Bioconductor suite, with colours generated using palettes from the RColorBrewer™ package (Neuwirth, 2011). Prior to clustering and visualization, individual gene expression across profiles was standardized to have zero mean and unit variance.

Differential Expression

The SAM algorithm (Tusher et al., 2001), as implemented in the R/Bioconductor package siggenes (Schwender, 2012), was applied to assess statistical significance of differential gene expression across discrete profile groups. Significance values obtained from multiple hypothesis tests were corrected for false-discovery by permutation as implemented in the SAM algorithm.

The Enrichr (Chen et al., 2013) tool (available at amp.pharm.mssm.eduEnrichr/) was used to assess statistical over-representation of gene categories among genes deemed differentially regulated. The tool combines 35 gene set libraries sorted by categories including transcription, pathways, ontologies, diseases, etc. and totaling 31,026 gene-sets.

Survival Analysis

The BioNet algorithm (Beisser et al., 2010), as implemented in the related Bioconductor package, was applied to the combined output of statistical tests for differential expression between refractory versus non-refractory best response groups and gene expression correlation with PFS and used the Human Interactome obtained from HINT (Das and Yu, 2012). Optimal response-related sub-networks were visualized via the Cytoscape platform (Saito et al., 2012; Shannon et al., 2003; Smoot et al., 2011).

The Reactome FI package (Wu and Stein, 2012) was implemented via Cytoscape and applied to Reactome annotation (Croft et al., 2011) imported with the software. The “microarray data analysis” option was applied, with database version 2012, absolute value for Pearson correlation, and an inflation parameter of 5.0 for the Markov Cluster Algorithm. After network generation, Biological Process and Pathway enrichment and survival analyses were generated using associated functionality of the package. Survival analyses were calculated per module upon import of corresponding PFS and censor information.

Cox regression models and Kaplan-Meier curves were performed using the R package survival (Terry M. Therneau, 2013; Terry M. Therneau and Patricia M. Grambsch, 2000).

Decomposition

Microarray gene expression profiles were decomposed using default functionality of the CellMix R/Bioconductor package (Gaujoux and Seoighe, 2013), which also provided collections of reference data used for decomposition. The decomposition method and reference collection of (Abbas et al., 2009) were applied to REV-DLC-001 profiles. Results were visualized and associated statistics calculated using native functionality of the R environment.

Results

Tables 3 and 4 provide lists of genes deemed significantly differentially regulated (empirical FDR 5%) pre-treatment, between patient groups determined as refractory and non-refractory further to Revlimid/lenalidomide therapy. Table 1 provides a list of genes deemed significantly upregulated (empirical FDR 5%) pre-treatment in patients non-refractory to further therapy relative to patients refractory to further Revlimid/lenalidomide therapy. Table 2 provides a list of genes deemed significantly downregulated (empirical FDR 5%) pre-treatment in patients non-refractory to further therapy relative to patients refractory to further Revlimid/lenalidomide therapy.

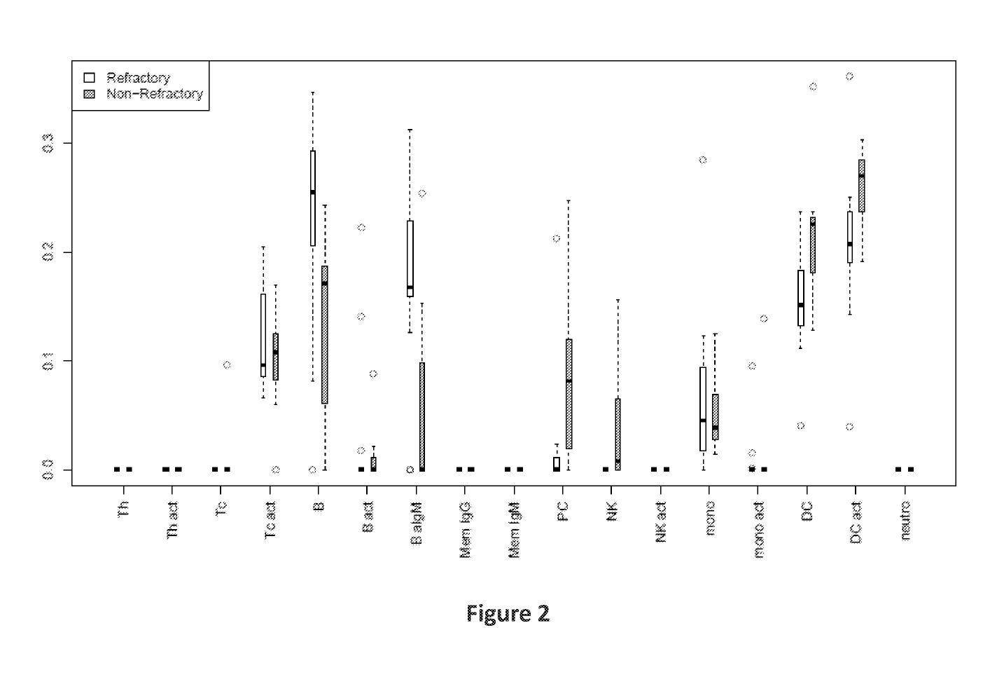

Analysis was performed on the FF sample profiles from the lenalidomide-arm and estimated relative proportions of the reference immune cell types were compared between profiles associated with binary lenalidomide response vs. non-response categories (see FIG. 2). The distinction between lenalidomide response profiles and the non-responding profiles is characterized by lower estimated B-cell proportions and clear increases in estimated proportions of plasma cells, dendritic cells, and, in a smaller subset of responding patients, NK cells.

It is of note that estimated proportions of monocytes and cytotoxic T-cells do not correlate strongly with Revlimid response, despite interpretations of the preceding analyses having suggested their presence. Rather, the patterns displayed appear more related to an earlier ‘host response’ (Monti et al., 2005) or immune infiltrate in combination with a corresponding reduction in particular B-cell populations—as in earlier interpretation of the combined THRLBCL (Van Loo et al., 2010)/host response (Monti et al., 2005) gene signatures.

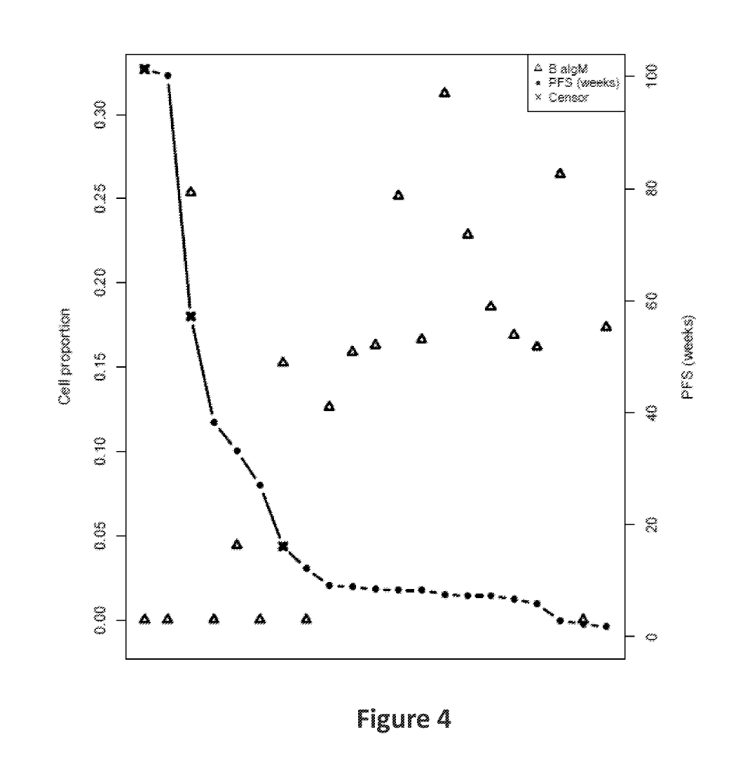

FIG. 4 displays estimated proportion of BCR-ligated B-cells (B aIgM) in baseline patient profiles plotted alongside corresponding progression free survival (PFS) further to lenalidomide treatment. The association between low estimated B aIgM proportion and relatively high PFS is striking, in particular in the knowledge that one patient with both low B aIgM proportion and low PFS (<3 weeks) died shortly after starting treatment.

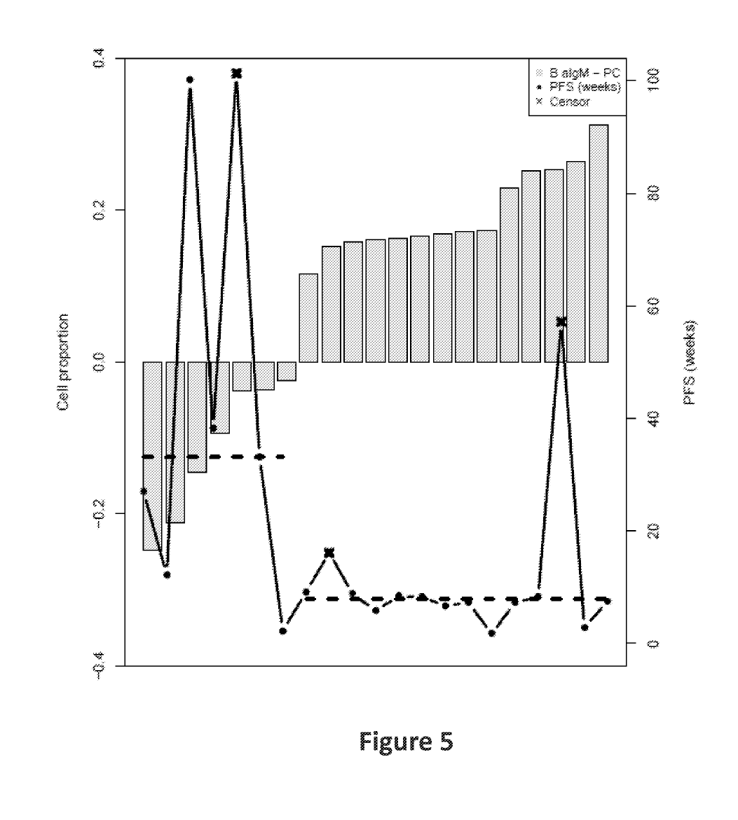

The estimated proportion of plasma cells displays the opposite tendency. This observation lends relevance to the decomposition data, as the two cell types are directly linked (Caven et al., 2007) and strong up-regulation of plasma cell marker differentiation marker PRDM1(BLIMP1) is observed in profiles associated with response to treatment. This pattern persists to the extent that the sign of a simple subtracted difference between estimated proportions of BCR-ligated B-cells and plasma cells across Revlimid arm profile decompositions (see FIG. 5 (lenalidomide-arm)) provides a statistically significant difference in PFS between two patient groups thus defined (Wilcoxon rank sum, p=0.04).

The response-related decomposition values for resting NK-cell proportions are interesting. Only four profiles are associated with non-zero estimated proportion of NK-cells, but these profiles are associated with PFS of 27, 31.9, 32.4 & 85.3 weeks. Clearly, were these estimations to bear out in practice (they include two very small estimated proportions, 0.114, 0.158, 0.004 & 0.002 respectively), the presence of NK cells in the tumor sample may provide a good indicator for Revlimid response enrichment.

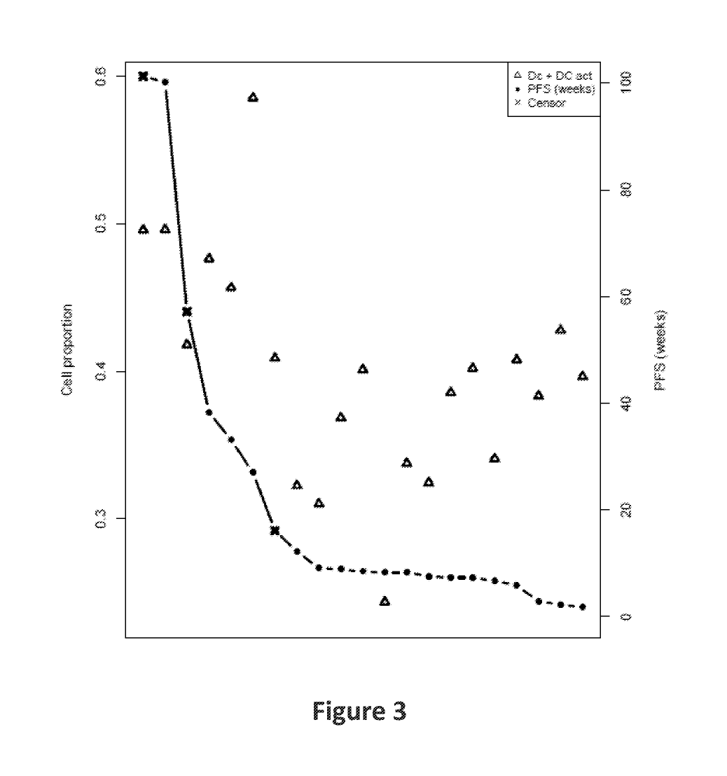

Finally, relative estimated proportions of dendritic cells are higher and more stable than for NK cells, although they lack the binary ‘on/off’ nature of BCR-ligated B-cell and plasma cell proportions in relation to best response categories. FIG. 3 displays the relationship between estimated DC proportion (resting+activated) and PFS. Taken together with the partially response-discriminant populations above, the results suggest potential for, for example, a focused flow cytometry screen for future patients more likely to respond to Revlimid treatment.

### REFERENCES

Abbas, A. R., Wolslegel, K., Seshasayce, D., Modrusan, Z., and Clark, H. F. (2009). Deconvolution of blood microarray data identifies cellular activation patterns in systemic lupus erythematosus. PloS One 4, e6098.
Abramson, J. S. (2006). T-cell/histiocyte-rich B-cell lymphoma: biology, diagnosis, and management. The Oncologist 11, 384-392.
Alizadeh, A. A., Eisen, M. B., Davis, R. E., Ma, C., Lossos, I. S., Rosenwald, A., Boldrick, J. C., Sabet, H., Tran, T., Yu, X., et al. (2000). Distinct types of diffuse large B-cell lymphoma Identi® ed by gene expression Pro® ling. 403.
Andersen, P. K., and Gill, R. D. (1982). Cox's regression model for counting processes: a large sample study. The Annals of Statistics 1100-1120.
Ashburner, M., Ball, C. A., Blake, J. A., Botstein, D., Butler, H., Cherry, J. M., Davis, A. P., Dolinski, K., Dwight, S. S., Eppig, J. T., et al. (2000). Gene ontology: tool for the unification of biology. The Gene Ontology Consortium. Nature Genetics 25, 25-29.
Barrett, T., Troup, D. B., Wilhite, S. E., Ledoux, P., Evangelista, C., Kim, I. F., Tomashevsky, M., Marshall, K. a, Phillippy, K. H., Sherman, P. M., et al. (2011). NCBI GEO: archive for functional genomics data sets—10 years on. Nucleic Acids Research 39, D1005-10.
Beisser, D., Klau, G. W., Dandekar, T., Miller, T., and Dittrich, M. T. (2010). BioNet: an R-Package for the functional analysis of biological networks. Bioinformatics 26, 1129-1130.
Bouabdallah, R. (2003). T-Cell/Histiocyte-Rich Large B-Cell Lymphomas and Classical Diffuse Large B-Cell Lymphomas Have Similar Outcome After Chemotherapy: A Matched-Control Analysis. Journal of Clinical Oncology 21, 1271-1277.
Caven, T. H., Sturgill, J. L., and Conrad, D. H. (2007). BCR Ligation antagonizes the IL-21 enhancement of anti-CD40/IL-4 Plasma cell differentiation and IgE production found in low density human B cell cultures. Cell Immunology 247, 49-58.
Chang, D. H., Liu, N., Klimek, V., Hassoun, H., Mazumder. A., Nimer, S. D., Jagannath, S., and Dhodapkar, M. V (2006). Enhancement of ligand-dependent activation of human natural killer T cells by lenalidomide: therapeutic implications. Blood 108, 618-621.
Chen, E. Y., Tan, C. M., Kou, Y., Duan, Q., Wang, Z., Meirelles, G. V., Clark, N. R., and Ma'ayan, A. (2013). Enrichr: interactive and collaborative HTML5 gene list enrichment analysis tool. BMC Bioinformatics 14, 128. Interim Analysis Report: September 2013
Croft, D., O'Kelly, G., Wu, G., Haw, R., Gillespie, M., Matthews, L., Caudy, M., Garapati, P., Gopinath, G., Jassal, B., et al. (2011). Reactome: a database of reactions, pathways and biological processes. Nucleic Acids Research 39, D691-D697.
Culhane, A. C., Schröder, M. S., Sultana, R., Picard, S. C., Martinelli, E. N., Kelly, C., Haibe-Kains, B., Kapushesky, M., St Pierre, A.-A., Flahive, W., et al. (2011). GeneSigDB: a manually curated database and resource for analysis of gene expression signatures. Nucleic Acids Research 40, D1060-6.
Das, J., and Yu, H. (2012). HINT: High-quality protein interactomes and their applications in understanding human disease. BMC Systems Biology 6, 92.
Dauguet, N., Fournié, J.-J., Poupot, R., and Poupot, M. (2010). Lenalidomide down regulates the production of interferon-gamma and the expression of inhibitory cytotoxic receptors of human Natural Killer cells. Cellular Immunology 264, 163-170.
Day, A. (2012). heatmap.plus: Heatmap with more sensible behavior.
Dittrich, M. T., Klau, G. W., Rosenwald, A., Dandekar, T., and Miller, T. (2008). Identifying functional modules in protein-protein interaction networks: an integrated exact approach. Bioinformatics (Oxford, England) 24, i223-31.
Van Dongen, S. M. (2000). Graph clustering by flow simulation.
Edgar, R., Domrachev, M., and Lash, A. E. (2002). Gene Expression Omnibus: NCBI gene expression and hybridization array data repository. Nucleic Acids Research 30, 207-210.
Gatter, K., and Pezzella, F. (2010). Diffuse large B-cell lymphoma. Diagnostic Histopathology 16, 69-81.
Gaujoux, R., and Seoighe, C. (2013). CellMix: a comprehensive toolbox for gene expression deconvolution. Bioinformatics (Oxford, England).
Gentleman, R. (2013). annotate: Annotation for microarrays.
Gentleman, R., Carey, V., Huber, W., and Hahne, F. (2013). genefilter: methods for filtering genes from microarray experiments. R Package Version 1-22.
Gong, T., Hartmann, N., Kohane, I. S., Brinkmann, V., Staedtler, F., Letzkus, M., Bongiovanni, S., and Szustakowski, J. D. (2011). Optimal deconvolution of transcriptional profiling data using quadratic programming with application to complex clinical blood samples. PloS One 6, e27156.
Görgün, G., Calabrese, E., Soydan, E., Hideshima, T., Perrone, G., Bandi, M., Cirstea, D., Santo, L., Hu, Y., Tai, Y.-T., et al. (2010). Immunomodulatory effects of lenalidomide and pomalidomide on interaction of tumor and bone marrow accessory cells in multiple myeloma. Blood 116, 3227-3237. Interim Analysis Report: September 2013
Irizarry, R. A., Bolstad, B. M., Collin, F., Cope, L. M., Hobbs, B., and Speed, T. P. (2003). Summaries of Affymetrix GeneChip probe level data. Nucleic Acids Research 31, e15.
Jacobson, T. a S., Lundahl, J., Mellstedt, H., and Moshfegh, A. (2011). Gene expression analysis using long-term preserved formalin-fixed and paraffin-embedded tissue of non-small cell lung cancer. International Journal of Oncology 38, 1075-1081.
Jego, G., Palucka, A. K., Blanck, J., Chalouni, C., Pascual, V., and Banchereau, J. (2002). Plasma Cell Differentiation through Type I Interferon and Interleukin 6. Immunity 19, 225-234.
Kauffmann, A., Gentleman, R., and Huber, W. (2009). arrayQualityMetrics—a bioconductor package for quality assessment of microarray data. Bioinformatics (Oxford, England) 25, 415-416.
Kotla, V., Goel, S., Nischal. S., Heuck, C., Vivek, K., Das, B., and Verma, A. (2009). Mechanism of action of lenalidomide in hematological malignancies. Journal of Hematology & Oncology 2, 36.
Lenz, G., Wright, G., Dave, S. S., Xiao, W., Powell, J., Zhao, H., Xu, W., Tan, B., Goldschmidt, N., Iqbal, J., et al. (2008). Stromal Gene Signatures in Large-B-Cell Lymphomas. New England Journal of Medicine 359, 2313-2323.
Van Loo, P., Tousseyn, T., Vanhentenrijk, V., Dierickx, D., Malecka, A., Vanden Bempt, I., Verhoef, G., Delabie, J., Marynen, P., Matthys, P., et al. (2010). T-cell/histiocyte-rich large B-cell lymphoma shows transcriptional features suggestive of a tolerogenic host immune response. Haematologica 95, 440-448.
McDaniel, J. M., Pinilla-Ibarz, J., and Epling-Burnette, P. K. (2012). Molecular Action of Lenalidomide in Lymphocytes and Hematologic Malignancies. Advances in Hematology 2012, 513702.
Mellor, A. L., and Munn, D. H. (2004). IDO expression by dendritic cells: tolerance and tryptophan catabolism. Nature Reviews. Immunology 4, 762-774.
Monti, S., Savage, K. J., Kutok, J. L., Feuerhake, F., Kurtin, P., Mihm, M., Wu, B., Pasqualucci, L., Neuberg, D., Aguiar, R. C. T., et al. (2005). Molecular profiling of diffuse large B-cell lymphoma identifies robust subtypes including one characterized by host inflammatory response. Blood 105, 1851-1861.
Mostafavi, S., Ray, D., Warde-farley, D., Grouios, C., and Morris, Q. (2008). Open Access GeneMANIA: a real-time multiple association network integration algorithm for predicting gene function. Genome Biology.
Neuwirth, E. (2011). RColorBrewer: ColorBrewer palettes. Ogata, H., Goto, S., Sato, K., Fujibuchi, W., Bono, H., and Kanehisa, M. (1999). KEGG: Kyoto Encyclopedia of Genes and Genomes. Nucleic Acids Research 27, 29-34.
Payvandi, F., Wu, L., Haley, M., Schafer, P. H., Zhang, L.-H., Chen, R. S., Muller, G. W., and Stirling, D. I. (2004). Immunomodulatory drugs inhibit expression of cyclooxygenase-2 from TNF-alpha, IL-Interim Analysis Report: September 2013
1beta, and LPS-stimulated human PBMC in a partially IL-10-dependent manner. Cellular Immunology 230, 81-88.
Pfreundschuh, M., Trimper, L., Kloess, M., Schmits, R., Feller, A. C., Rudolph, C., Reiser, M., Hossfeld, D. K., Metzner, B., Hasenclever, D., et al. (2004a). Two-weekly or 3-weekly CHOP chemotherapy with or without etoposide for the treatment of young patients with good-prognosis (normal LDH) aggressive lymphomas: results of the NHL-B1 trial of the DSHNHL. Blood 104, 626-633.
Pfreundschuh, M., Trimper, L., Kloess, M., Schmits, R., Feller, A. C., Ribe, C., Rudolph, C., Reiser, M., Hossfeld, D. K., Eimermacher, H., et al. (2004b). Two-weekly or 3-weekly CHOP chemotherapy with or without etoposide for the treatment of elderly patients with aggressive lymphomas: results of the NHL-B2 trial of the DSHNHL. Blood 104, 634-641.
Pittaluga, S., and Jaffe. E. S. (2010). T-cell/histiocyte-rich large B-cell lymphoma. Haematologica 95, 352-356.
Ramsay, A. G., Clear, A. J., Kelly, G., Fatah, R., Matthews, J., Macdougall, F., Lister, T. A., Lee, A. M., Calaminici, M., and Gribben, J. G. (2009). Follicular lymphoma cells induce T-cell immunologic synapse dysfunction that can be repaired with lenalidomide: implications for the tumor microenvironment and immunotherapy. Blood 114, 4713-4720.
Ramsay, A. G., Clear, A. J., Fatah, R., and Gribben, J. G. (2012). Multiple inhibitory ligands induce impaired T-cell immunologic synapse function in chronic lymphocytic leukemia that can be blocked with lenalidomide: establishing a reversible immune evasion mechanism in human cancer. Blood 120, 1412-1421.
Ramsay, A. G., Evans, R., Kiaii, S., Svensson, L., Hogg, N., and Gribben, J. G. (2013). Chronic lymphocytic leukemia cells induce defective LFA-1-directed T-cell motility by altering Rho GTPase signaling that is reversible with lenalidomide. Blood 121, 2704-2714.
Saito, R., Smoot, M. E., Ono, K., Ruscheinski, J., Wang, P.-L., Lotia, S., Pico, A. R., Bader, G. D., and Ideker, T. (2012). A travel guide to Cytoscape plugins. Nat Meth 9, 1069-1076.
Schulz, A., Dürr, C., Zenz, T., Döhner, H., Stilgenbauer, S., Lichter, P., and Seiffert, M. (2013). Lenalidomide reduces survival of chronic lymphocytic leukemia cells in primary cocultures by altering the myeloid microenvironment. Blood 121, 2503-2511.
Schwender, H. (2012). siggenes: Multiple testing using SAM and Efron's empirical Bayes approaches.
Sehn, L. H., Donaldson, J., Chhanabhai, M., Fitzgerald, C., Gill, K., Klasa, R., MacPherson, N., O'Reilly, S., Spinelli, J. J., Sutherland, J., et al. (2005). Introduction of combined CHOP plus rituximab therapy dramatically improved outcome of diffuse large B-cell lymphoma in British Columbia. Journal of Clinical Oncology: Official Journal of the American Society of Clinical Oncology 23, 5027-5033. Interim Analysis Report: September 2013
Shannon, P., Markiel, A., Ozier, O., Baliga, N. S., Wang, J. T., Ramage, D., Amin. N., Schwikowski, B., and Ideker, T. (2003). Cytoscape: a software environment for integrated models of biomolecular interaction networks. Genome Research 13, 2498-2504.
Smoot, M., Ono, K., Ruscheinski, J., Wang, P.-L., and Ideker, T. (2011). Cytoscape 2.8: new features for data integration and network visualization. Bioinformatics 27, 431-432.
Swerdllow, S. H., Campo, E., and Harris, N. L. (2008). WHO classification of tumours of haematopoietic and lymphoid tissues (France: ARC Press, 2008).
Terry M. Themeau (2013). A Package for Survival Analysis in S. Terry M. Therneau, and Patricia M. Grambsch (2000). Modeling Survival Data: Extending the Cox Model (New York: Springer).
Tusher, V. G., Tibshirani, R., and Chu, G. (2001). Significance analysis of microarrays applied to the ionizing radiation response. Proceedings of the National Academy of Sciences of the United States of America 98, 5116-5121.
Warnes, G. R., Bolker, B., Bonebakker, L., Gentleman, R., Liaw, W. H. A., Lumley, T., Maechler, M., Magnusson, A., Moeller. S., Schwartz, M., et al. (2013). gplots: Various R programming tools for plotting data.
Wu, G., and Stein, L. (2012). A network module-based method for identifying cancer prognostic signatures. Genome Biology 13, R112.
Wu, G., Feng, X., and Stein, L. (2010). A human functional protein interaction network and its application to cancer data analysis. Genome Biology 11, R53.
Xu, Y., Li, J., Ferguson, G. D., Mercurio, F., Khambatta, G., Morrison, L., Lopez-Girona, A., Corral, L. G., Webb, D. R., Bennett, B. L., et al. (2009). Immunomodulatory drugs reorganize cytoskeleton by modulating Rho GTPases. Blood 114, 338-345.
Zhu, Y. X., Kortuem, K. M., and Stewart, a K. (2012). Molecular mechanism of action of immune-modulatory drugs thalidomide, lenalidomide and pomalidomide in multiple myeloma. Leukemia & Lymphoma.

Table 1, Table, 2, Table 3 and Table 4 are provided below.

TABLE 1

| | | | | | mean_ | mean_ | Chro- | chrom_ | |
|---|---|---|---|---|---|---|---|---|---|
| probeset | gene_symbol | ensembl_id | q.value | logFC | nonRef | Ref | mosome | band | gene_description |
| 228754_at | SLC6A6 | ENSG00000131389 | 0 | 0.9754805 | 8.981006 | 8.005526 | 3 | p25.1 | solute carrier family 6 (neurotransmitter transporter, taurine), member 6 |
| | | | | | | | | | [Source: HGNC Symbol; Acc: 11052] |
| 238327_at | ODF3B | ENSG00000177989 | 0.00273 | 1.0304117 | 8.897771 | 7.86736 | 22 | q13.33 | outer dense fiber of sperm tails 3B [Source: HGNC Symbol; Acc: 34388] |
| 208683_at | CAPN2 | ENSG00000162909 | 0.00273 | 2.0591386 | 9.820198 | 7.761059 | 1 | q41 | calpain 2, (m/ll) large subunit [Source: HGNC Symbol; Acc: 1479] |
| 218648_at | CRTC3 | ENSG00000140577 | 0.00273 | 1.3932573 | 9.008044 | 7.614786 | 15 | q26.1 | CREB regulated transcription coactivator 3 [Source: HGNC Symbol; Acc: 26148] |
| 225146_at | FAM219A | ENSG00000164970 | 0.00273 | 0.7957467 | 8.091946 | 7.296199 | 9 | p13.3 | family with sequence similarity 219, member A [Source: HGNC Symbol; Acc: 19920] |
| 218589_at | LPAR6 | ENSG00000139679 | 0.00273 | 2.4225735 | 9.179664 | 6.75709 | 13 | q14.2 | lysophosphatidic acid receptor 6 [Source: HGNC Symbol; Acc: 15520] |
| 217762_s_at | RAB31 | ENSG00000168461 | 0.00273 | 1.7495761 | 10.13227 | 8.382693 | 18 | p11.22 | RAB31, member RAS oncogene family [Source: HGNC Symbol; Acc: 9771] |
| 201506_at | TGFBI | ENSG00000120708 | 0.00273 | 1.8055477 | 11.4844 | 9.678852 | 5 | q31.1 | transforming growth factor, beta-induced, 68 kDa [Source: HGNC |
| | | | | | | | | | Symbol; Acc: 11771] |
| 224862_at | GNAQ | ENSG00000156052 | 0.00273 | 2.0253634 | 6.97061 | 4.945246 | 9 | q21.2 | guanine nucleotide binding protein (G protein), q polypeptide [Source: HGNC |
| | | | | | | | | | Symbol; Acc: 4390] |
| 201425_at | ALDH2 | ENSG00000111275 | 0.00273 | 1.9293787 | 10.60165 | 8.672267 | 12 | q24.12 | aldehyde dehydrogenase 2 family (mitochondrial) [Source: HGNC |
| | | | | | | | | | Symbol; Acc,: 404] |
| 1555851_s_at | SEPW1 | ENSG00000178980 | 0.00312 | 1.5331244 | 10.54942 | 9.016292 | 19 | q13.33 | selenoprotein W, 1 [Source: HGNC Symbol; Acc: 10752] |
| 205241_at | SCO2 | ENSG00000130489 | 0.00312 | 0.8920118 | 9.145278 | 8.253267 | 22 | q13.33 | SCO2 cytochrome c oxidase assembly protein [Source: HGNC Symbol; Acc: 10604] |
| 218552_at | ECHDC2 | ENSG00000121310 | 0.00312 | 1.3353136 | 9.234133 | 7.89882 | 1 | p32.3 | enoyl CoA hydratase domain containing 2 [Source: HGNC Symbol; Acc: 23408] |
| 55662_at | C10orf76 | ENSG00000120029 | 0.00312 | 0.7212711 | 8.212153 | 7.490882 | 10 | q24.32 | chromosome 10 open reading frame 76 [Source: HGNC Symbol; Acc: 257881 |
| 204773_at | IL11RA | ENSG00000137070 | 0.004437 | 0.9173902 | 7.610983 | 6.693593 | 9 | p13.3 | interleukin 11 receptor, alpha [Source: HGNC Symbol; Acc: 5967] |
| 202600_s_at | NRIP1 | ENSG00000180530 | 0.004437 | 1.662366 | 7.562455 | 5.900089 | 21 | q21.1 | nuclear receptor interacting protein 1 [Source: HGNC Symbol; Acc: 8001] |
| 221802_s_at | KIAA1598 | ENSG00000187164 | 0.004818 | 1.8894563 | 8.343765 | 6.454309 | 10 | q25.3 | KIAA1598 [Source: HGNC Symbol; Acc: 29319] |
| 226000_at | CTTNBP2NL | ENSG00000143079 | 0.005157 | 1.4672863 | 7.674825 | 6.207539 | 1 | p13.2 | CTTNBP2 N-terminal like [Source: HGNC Symbol; Acc: 25330] |
| 222484_s_at | CXCL14 | ENSG00000145824 | 0.005173 | 3.4617357 | 11.01983 | 7.558095 | 5 | q31.1 | chemokine (C-X-C motif) ligand 14 [Source: HGNC Symbol; Acc: 10640] |
| 201012_at | ANXA1 | ENSG00000135046 | 0.005188 | 1.8681422 | 10.78464 | 8.916494 | 9 | q21.13 | annexin A1 [Source: HGNC Symbol; Acc: 533] |
| 218854_at | DSE | ENSG00000111817 | 0.006453 | 1.6321343 | 9.397392 | 7.765258 | 6 | q22.1 | dermatan sulfate epimerase [Source: HGNC Symbol; Acc: 21144] |
| 214040_s_at | GSN | ENSG00000148180 | 0.006453 | 1.3451184 | 10.2628 | 8.917685 | 9 | q33.2 | gelsolin [Source: HGNC Symbol; Acc: 4620] |
| 201302_at | ANXA4 | ENSG00000196975 | 0.006598 | 1.7017558 | 9.377174 | 7.675418 | 2 | p13.3 | annexin A4 [Source: HGNC Symbol; Acc: 542] |
| 208923_at | CYFIP1 | ENSG00000068793 | 0.006598 | 1.4183371 | 9.308029 | 7.889692 | 15 | q11.2 | cytoplasmic FMR1 interacting protein 1 [Source: HGNC Symbol; Acc: 13759] |
| 224414_s_at | CARD6 | ENSG00000132357 | 0.007208 | 1.298678 | 8.497051 | 7.198373 | 5 | p13.1 | caspase recruitment domain family, member 6 [Source: HGNC Symbol; Acc: 16394] |
| 205945_at | IL6R | ENSG00000160712 | 0.008097 | 1.7659924 | 8.874117 | 7.108125 | 1 | q21.3 | interleukin 6 receptor [Source: HGNC Symbol; Acc: 6019] |
| 200765_x_at | CTNNA1 | ENSG00000044115 | 0.008097 | 1.1179416 | 9.63084 | 8.512898 | 5 | q31.2 | catenin (cadherin-associated protein), alpha 1, 102 kDa [Source: HGNC |
| | | | | | | | | | Symbol; Acc: 2509] |
| 223228_at | LDOC1L | ENSG00000188636 | 0.008097 | 1.239346 | 8.444984 | 7.205638 | 22 | q13.31 | leucine zipper, down-regulated in cancer 1-like [Source: HGNC Symbol; Acc: 13343] |
| 225842_at | PHLDA1 | ENSG00000139289 | 0.008191 | 1.472676 | 8.29771 | 6.825034 | 12 | q21.2 | pleckstrin homology-like domain, family A, member 1 [Source: HGNC |
| | | | | | | | | | Symbol; Acc: 8933] |
| 201348_at | GPX3 | ENSG00000211445 | 0.008351 | 2.1699179 | 11.32422 | 9.154304 | 5 | q33.1 | glutathione peroxidase 3 (plasma) [Source: HGNC Symbol; Acc: 4555] |
| 219885_at | SLFN12 | ENSG00000172123 | 0.008351 | 1.2351533 | 6.097789 | 4.862636 | 17 | q12 | schlafen family member 12 [Source: HGNC Symbol; Acc: 25500] |
| 212830_at | MEGF9 | ENSG00000106780 | 0.008351 | 1.2431752 | 6.734085 | 5.490909 | 9 | q33.2 | multiple EFG-like-domains 9 [Source: HGNC Symbol; Acc: 3234] |
| 202687_s_at | TNFSF10 | ENSG00000121858 | 0.008425 | 1.5293527 | 11.10527 | 9.57592 | 3 | q26.31 | tumor necrosis factor (ligand) superfamily, member 10 [Source: HGNC |
| | | | | | | | | | Symbol; Acc: 11925] |
| 217731_s_at | ITM2B | ENSG00000136156 | 0.009101 | 1.4493355 | 10.05153 | 8.602193 | 13 | q14.2 | integral membrane protein 28 [Source: HGNC Symbol; Acc: 6174] |
| 218694_at | ARMCX1 | ENSG00000126947 | 0.009456 | 1.4991554 | 7.418732 | 5.919576 | X | q22.1 | armadillo repeat containing, X-linked 1 [Source: HGNC Symbol; Acc: 18073] |
| 202944_at | NAGA | ENSG00000198951 | 0.009456 | 0.9946444 | 8.604684 | 7.61004 | 22 | q13.2 | N-acetylgalactosaminidase, alpha- [Source: HGNC Symbol; Acc: 7631] |
| 212522_at | PDE8A | ENSG00000073417 | 0.009456 | 1.2124713 | 8.751053 | 7.538532 | 15 | q25.3 | phosphodiesterase 8A [Source: HGNC Symbol; Acc: 8793] |
| 226247_at | PLEKHA1 | ENSG00000107679 | 0.009456 | 1.762189 | 8.231589 | 6.4694 | 10 | q26.13 | pleckstrin homology domain containing, family A (phosphoinositide binding |
| | | | | | | | | | specific) member 1 [Source: HGNC Symbol; Acc: 14335] |
| 243423_at | TNIP1 | ENSG00000145901 | 0.009621 | 1.0393627 | 6.139027 | 5.099664 | 5 | q33.1 | TNFAIP3 interacting protein 1 [Source: HGNC Symbol; Acc: 16903] |
| 229074_at | EHD4 | ENSG00000103966 | 0.00995 | 1.116796 | 8.728409 | 7.611613 | 15 | q15.1 | EH-domain containing 4 [Source: HGNC Symbol; Acc: 3245] |
| 222154_s_at | SPATS2L | ENSG00000196141 | 0.00995 | 1.2398395 | 9.909155 | 8.669316 | 2 | q33.1 | spermatogenesis associated, serine-rich 2-like [Source: HGNC Symbol; Acc: 24574] |
| 200710_at | ACADVL | ENSG00000072778 | 0.011037 | 0.8720084 | 9.456312 | 8.584304 | 17 | p13.1 | acyl-CoA dehydrogenase, very long chain [Source: HGNC Symbol; Acc: 92] |
| 228791_at | LOC100129502 | NA | 0.011037 | 1.7938919 | 8.411267 | 6.617375 | NA | NA | NA |
| 208634_s_at | MACF1 | ENSG00000127603 | 0.011768 | 1.2346253 | 10.26299 | 9.028368 | 1 | p34.3 | microtubule-actin crosslinking factor 1 [Source: HGNC Symbol; Acc: 13664] |
| 242487_at | CC2D1B | ENSG00000154222 | 0.011768 | 0.5238144 | 6.843606 | 6.319792 | 1 | p32.3 | coiled-coil and C2 domain containing 1B [Source: HGNC Symbol; Acc: 29336] |
| 218559_s_at | MAFB | ENSG00000204103 | 0.011768 | 1.8491222 | 11.31545 | 9.466329 | 20 | q12 | v-maf avian musculoaponeurotic fibrosarcoma oncogene homolog B |
| | | | | | | | | | [Source: HGNC Symbol; Acc: 6408] |
| 201360_at | CST3 | ENSG00000101439 | 0.011768 | 1.3533248 | 11.64386 | 10.29054 | 20 | p11.21 | cystatin C [Source: HGNC Symbol; Acc: 2475] |
| 206101_at | ECM2 | ENSG00000106823 | 0.011768 | 1.7286086 | 7.156511 | 5.427902 | 9 | q22.31 | extracellular matrix protein 2, female organ and adipocyte specific [Source: HGNC |
| | | | | | | | | | Symbol; Acc: 3154] |
| 218004_at | BSDC1 | ENSG00000160058 | 0.011768 | 0.5416922 | 8.948162 | 8.40647 | 1 | p35.1 | BSD domain containing 1 [Source: HGNC Symbol; Acc: 25501] |
| 36129_at | SGSM2 | ENSG00000141258 | 0.011768 | 0.89385 | 9.48007 | 8.58622 | 17 | p13.3 | small G protein signaling modulator 2 [Source: HGNC Symbol; Acc: 29026] |
| 227889_at | LPCAT2 | ENSG00000087253 | 0.011768 | 1.9822482 | 7.953706 | 5.971458 | 16 | q12.2 | lysophosphatidylcholine acyltransferase 2 [Source: HGNC Symbol; Acc: 26032] |
| 218043_s_at | AZI2 | ENSG00000163512 | 0.011768 | 0.8638989 | 6.644285 | 5.780336 | 3 | p24.1 | 5-azacytidine induced 2 [Source: HGNC Symbol; Acc: 24002] |
| 218066_at | SLC12A7 | ENSG00000113504 | 0.011768 | 1.0053443 | 8.912722 | 7.907378 | 5 | p15.33 | solute carrier family 12 (potassium/chloride transporters), member 7 |
| | | | | | | | | | [Source: HGNC Symbol; Acc: 10915] |
| 58780_s_at | ARHGEF40 | ENSG00000165801 | 0.011847 | 1.4859821 | 7.235422 | 5.74944 | 14 | q11.2 | Rho guanine nucleotide exchange factor (GEF) 40 [Source: HGNC |
| | | | | | | | | | Symbol; Acc: 25516] |
| 200660_at | S100A11 | ENSG00000163191 | 0.01233 | 1.3882864 | 11.61066 | 10.22237 | 1 | q21.3 | S100 calcium binding protein A11 [Source: HGNC Symbol; Acc: 10488] |
| 212907_at | SLC30A1 | ENSG00000170385 | 0.01233 | 1.1713121 | 9.350467 | 8.179154 | 1 | q32.3 | solute carrier family 30 (zinc transporter), member 1 [Source: HGNC |
| | | | | | | | | | Symbol; Acc: 11012] |
| 208999_at | SEPT8 | ENSG00000164402 | 0.012828 | 1.1133968 | 8.412743 | 7.299346 | 5 | q31.1 | septin 8 [Source: HGNC Symbol; Acc: 16511] |
| 208949_s_at | LGALS3 | ENSG00000131981 | 0.012937 | 1.4663704 | 11.76909 | 10.30272 | 14 | q22.3 | lectin, galactoside-binding, soluble, 3 [Source: HGNC Symbol; Acc: 6563] |
| 218311_at | MAP4K3 | ENSG00000011566 | 0.012937 | 1.467988 | 7.560719 | 6.092731 | 2 | p22.1 | mitogen-activated protein kinase kinase kinase kinase 3 [Source: HGNC |
| | | | | | | | | | Symbol; Acc: 6865] |
| 203518_at | LYST | ENSG00000143669 | 0.013774 | 1.7813892 | 8.315307 | 6.533918 | 1 | q42.3 | lysosomal trafficking regulator [Source: HGNC Symbol; Acc: 19681 |
| 204137_at | GPR137B | ENSG00000077585 | 0.01568 | 2.0887506 | 9.969767 | 7.881016 | 1 | q42.3 | G protein-coupled receptor 137B [Source: HGNC Symbol; Acc: 11862] |
| 222217_s_at | SLC27A3 | ENSG00000143554 | 0.01568 | 0.9016644 | 8.45229 | 7.550626 | 1 | q21.3 | solute carrier family 27 (fatty acid transporter), member 3 [Source: HGNC |
| | | | | | | | | | Symbol; Acc: 10997] |
| 201505_at | LAMB1 | ENSG00000091136 | 0.015689 | 1.8347161 | 9.325408 | 7.490692 | 7 | q31.1 | laminin, beta 1 [Source: HGNC Symbol; Acc: 6486] |
| 217728_at | S100A6 | ENSG00000197956 | 0.016157 | 0.9618026 | 11.17652 | 10.21472 | 1 | q21.3 | S100 calcium binding protein A6 [Source: HGNC Symbol; Acc: 10496] |
| 225483_at | VPS26B | ENSG00000151502 | 0.01616 | 0.7314643 | 8.008599 | 7.277135 | 11 | q25 | vacuolar protein sorting 26 homolog B (S. pombe) [Source: HGNC |
| | | | | | | | | | Symbol; Acc: 28119] |
| 202686_s_at | AXL | ENSG00000167601 | 0.016163 | 2.1648837 | 9.749176 | 7.584292 | 19 | q13.2 | AXL receptor tyrosine kinase [Source: HGNC Symbol; Acc: 905] |
| 227276_at | PLXDC2 | ENSG00000120594 | 0.016167 | 1.9691918 | 9.857934 | 7.888742 | 10 | p12.31 | plexin domain containing 2 [Source: HGNC Symbol; Acc: 2013] |
| 228185_at | ZNF25 | ENSG00000175395 | 0.016167 | 0.9293975 | 6.510839 | 5.581442 | 10 | p11.1 | zinc finger protein 25 [Source: HGNC Symbol; Acc: 13043] |
| 217892_s_at | LIMA1 | ENSG00000050405 | 0.016167 | 1.8593217 | 9.968366 | 8.109044 | 12 | q13.12 | LIM domain and actin binding 1 [Source: HGNC Symbol; Acc: 24636] |
| 202727_s_at | IFNGR1 | ENSG00000027697 | 0.016167 | 1.2033225 | 9.855228 | 8.651905 | 6 | q23.3 | interferon gamma receptor 1 [Source: HGNC Symbol; Acc: 5439] |
| 212112_s_at | STX12 | ENSG00000117758 | 0.016167 | 0.8115894 | 8.768046 | 7.956457 | 1 | p35.3 | syntaxin 12 [Source: HGNC Symbol; Acc: 11430] |
| 203789_s_at | SEMA3C | ENSG00000075223 | 0.016167 | 1.8263518 | 6.851145 | 5.024793 | 7 | q21.11 | sema domain, immunoglobulin domain (lg), short basic domain, secreted, |
| | | | | | | | | | (semaphorin) 3C [Source: HGNC Symbol; Acc: 10725] |
| 200677_at | PTTG1IP | ENSG00000183255 | 0.016167 | 0.9443082 | 10.81552 | 9.871214 | 21 | q22.3 | pituitary tumor-transforming 1 interacting protein [Source: HGNC |
| | | | | | | | | | Symbol; Acc: 13524] |
| 222876_s_at | ADAP2 | ENSG00000184060 | 0.016167 | 1.230284 | 9.947909 | 8.717625 | 17 | q11.2 | ArfGAP with dual PH domains 2 [Source: HGNC Symbol; Acc; 16487] |
| 210145_at | PLA2G4A | ENSG00000116711 | 0.016167 | 1.018587 | 5.59769 | 4.579103 | 1 | q31.1 | phospholipase A2, group IVA (cytosolic, calcium-dependent) [Source: HGNC |
| | | | | | | | | | Symbol; Acc: 9035] |
| 208109_s_at | LINC00597 | NA | 0.016167 | 1.2522138 | 6.056933 | 4.804719 | NA | NA | NA |
| 209651_at | TGFB1I1 | ENSG00000140682 | 0.016167 | 1.6390689 | 8.383831 | 6.744762 | 16 | p11.2 | transforming growth factor beta 1 induced transcript 1 [Source: HGNC |
| | | | | | | | | | Symbol; Acc: 11767] |
| 212698_s_at | SEPT10 | ENSG00000186522 | 0.016167 | 2.0293866 | 6.787502 | 4.758115 | 2 | q13 | septin 10 [Source: HGNC Symbol; Acc: 14349] |
| 212526_at | SPG20 | ENSG00000133104 | 0.016167 | 1.308041 | 6.974473 | 5.666432 | 13 | q13.3 | spastic paraplegia 20 (Troyer syndrome) [Source: HGNC Symbol; Acc: 18514] |
| 209684_at | RIN2 | ENSG00000132669 | 0.016167 | 1.9484987 | 9.25026 | 7.301761 | 20 | p11.23 | Ras and Rab interactor 2 [Source: HGNC Symbol; Acc: 18750] |
| 223204_at | FAM198B | ENSG00000164125 | 0.016167 | 1.5567047 | 7.718117 | 6.161412 | 4 | q32.1 | family with sequence similarity 198, member B [Source: HGNC Symbol; |
| | | | | | | | | | Acc: 25312] |
| 200673_at | LAPTM4A | ENSG00000068697 | 0.016167 | 1.052583 | 10.80128 | 9.748695 | 2 | p24.1 | lysosomal protein transmembrane 4 alpha [Source: HGNC Symbol; Acc: 6924] |
| 225384_at | DOCK7 | ENSG00000116641 | 0.016167 | 0.820345 | 7.335351 | 6.515006 | 1 | p31.3 | dedicator of cytokinesis 7 [Source: HGNC Symbol,Acc: 19190] |
| 209210_s_at | FERMT2 | ENSG00000073712 | 0.016167 | 1.600225 | 8.650583 | 7.050358 | 14 | q22.1 | fermitin family member 2 [Source: HGNC Symbol; Acc: 15767] |
| 201798_s_at | MYOF | ENSG00000138119 | 0.016167 | 1.9337214 | 9.90089 | 7.967169 | 10 | q23.33 | myoferlin [Source: HGNC Symbol; Acc: 3656] |
| 225949_at | NRBP2 | ENSG00000185189 | 0.016167 | 1.1978158 | 8.229623 | 7.031807 | 8 | q24.3 | nuclear receptor binding protein 2 (Source: HGNC Symbol; Acc: 19339] |
| 208924_at | RNF11 | ENSG00000123091 | 0.016167 | 1.3126563 | 7.905885 | 6.593229 | 1 | p32.3 | ring finger protein 11 [Source: HGNC Symbol; Acc: 10056] |
| 209004_s_at | FBXL5 | ENSG00000118564 | 0.016167 | 1.0838058 | 10.00546 | 8.921656 | 4 | p15.32 | F-box and leucine-rich repeat protein 5 [Source: HGNC Symbol; Acc: 13602] |
| 226743_at | SLFN11 | ENSG00000172716 | 0.016167 | 1.6530276 | 8.752878 | 7.099851 | 17 | q12 | schlafen family member 11 [Source: HGNC Symbol; Acc: 26633] |
| 228573_at | ANTXR2 | ENSG00000163297 | 0.016167 | 1.2231191 | 6.502217 | 5.279098 | 4 | q21.21 | anthrax toxin receptor 2 [Source: HGNC Symbol; Acc: 21732] |
| 222065_s_at | FLII | ENSG00000177731 | 0.016167 | 0.7794236 | 9.687503 | 8.90808 | 17 | p11.2 | flightless I homolog (Drosophila) [Source: HGNC Symbol; Acc: 3750] |
| 212989_at | SGMS1 | ENSG00000198964 | 0.016167 | 1.1804345 | 5.341958 | 4.161523 | 10 | q11.23 | sphingomyelin synthase 1 [Source: HGNC Symbol; Acc: 29799] |
| 202228_s_at | NPTN | ENSG00000156642 | 0.016167 | 1.1313461 | 9.703866 | 8.57252 | 15 | q24.1 | neuroplastin [Source: HGNC Symbol; Acc: 17867] |
| 227761_at | MYO5A | ENSG00000197535 | 0.016167 | 1.4777088 | 8.28231 | 6.804601 | 15 | q21.2 | myosin VA (heavy chain 12, myoxin) [Source: HGNC Symbol; Acc: 7602] |
| 202133_at | WWTR1 | ENSG00000018408 | 0.016167 | 1.9825407 | 8.759155 | 6.776615 | 3 | q25.1 | WW domain containing transcription regulator 1 [Source: HGNC |
| | | | | | | | | | Symbol; Acc: 24042] |
| 209960_at | HGF | ENSG00000019991 | 0.016167 | 1.2162213 | 4.738985 | 3.522763 | 7 | q21.11 | hepatocyte growth factor (hepapoietin A; scatter factor) [Source: HGNC |
| | | | | | | | | | Symbol; Acc: 4893] |
| 212203_x_at | IFITM3 | ENSG00000142089 | 0.016167 | 0.9994654 | 12.83133 | 11.83186 | 11 | p15.5 | interferon induced transmembrane protein 3 [Source: HGNC Symbol; Acc: 5414] |
| 212158_at | SDC2 | ENSG00000169439 | 0.016167 | 1.6948793 | 8.399184 | 6.704305 | 8 | q22.1 | syndecan 2 [Source: HGNC Symbol; Acc: 10659] |
| 224797_at | ARRDC3 | ENSG00000113369 | 0.016167 | 1.2808966 | 7.04992 | 5.769023 | 5 | q14.3 | arrestin domain containing 3 [Source: HGNC Symbol; Acc: 29263] |
| 203124_s_at | SLC11A2 | ENSG00000110911 | 0.016167 | 1.3149816 | 8.210637 | 6.895655 | 12 | q13.12 | solute carrier family 11 (proton-coupled divalent metal ion transporters), member |
| | | | | | | | | | 2 [Source: HGNC Symbol; Acc: 10908] |
| 1553034_at | SDCCAG8 | ENSG00000054282 | 0.016167 | 0.6479854 | 7.598444 | 6.950458 | 1 | q43 | serologically defined colon cancer antigen 8 [Source: HGNC Symbol; Acc: 10671] |
| 226111_s_at | ZNF385A | ENSG00000161642 | 0.016167 | 1.0612964 | 9.529822 | 8.46525 | 12 | q13.13 | zinc finger protein 385A [Source: HGNC Symbol; Acc: 17521] |
| 231579_s_at | TIMP2 | ENSG00000035862 | 0.016167 | 1.9509024 | 11.29034 | 9.339442 | 17 | q25.3 | TIMP metallopeptidase inhibitor 2 [Source: HGNC Symbol; Acc: 11821] |
| 202007_at | NID1 | ENSG00000116962 | 0.016167 | 1.6171384 | 7.967908 | 6.350769 | 1 | q42.3 | nidogen 1 [Source: HGNC Symbol; Acc: 7821] |
| 201681_s_at | DLG5 | ENSG00000151208 | 0.016382 | 1.584355 | 7.680377 | 6.096022 | 10 | q22.3 | discs, large homolog 5 (Drosophila) [Source: HGNC Symbol; Acc: 2904] |
| 212761_at | ICF7L2 | ENSG00000148737 | 0.016678 | 1.5655854 | 7.595306 | 6.02972 | 10 | q25.2 | transcription factor 7-like 2 (T-cell specific, HMG-box) [Source: HGNC |
| | | | | | | | | | Symbol; Acc: 11641] |
| 224983_at | SCARB2 | ENSG00000138760 | 0.016923 | 1.3192641 | 9.808333 | 8.489069 | 4 | q21.1 | scavenger receptor class B, member 2 [Source: HGNC Symbol; Acc: 1665] |
| 218706_s_at | GRAMD3 | ENSG00000155324 | 0.016923 | 1.1289944 | 6.584781 | 5.455786 | 5 | q23.2 | GRAM domain containing 3 [Source: HGNC Symbol; Acc: 24911] |
| 203595_s_at | IFIT5 | ENSG00000152778 | 0.017039 | 1.3141225 | 8.100951 | 6.786828 | 10 | q23.31 | interferon-induced protein with tetratricopeptide repeats 5 [Source: HGNC |
| | | | | | | | | | Symbol; Acc: 13328] |
| 225133_at | KLF3 | ENSG00000109787 | 0.017271 | 1.8771599 | 7.744133 | 5.866973 | 4 | p14 | Kruppel-like factor 3 (basic) [Source: HGNC Symbol; Acc: 16516] |
| 204396_s_at | GRK5 | ENSG00000198873 | 0.017458 | 0.9777677 | 8.134717 | 7.156949 | 10 | q26.11 | G protein-coupled receptor kinase 5 [Source: HGNC Symbol; Acc: 4544] |
| 210105_s_at | FYN | ENSG00000010810 | 0.017653 | 1.6156589 | 10.56934 | 8.953681 | 6 | q21 | FYN oncogene related to SRC, FGR, YES [Source: HGNC Symbol; Acc: 4037] |
| 201218_at | CTBP2 | ENSG00000175029 | 0.017653 | 1.464242 | 7.445366 | 5.981124 | 10 | q26.13 | C-terminal binding protein 2 [Source: HGNC Symbol; Acc: 2495] |
| 203973_s_at | CEBPD | ENSG00000221869 | 0.017653 | 1.6141966 | 10.46813 | 8.85393 | 8 | q11.21 | CCAAT/enhancer binding protein (C/EBP), delta [Source: HGNC |
| | | | | | | | | | Symbol; Acc: 1835] |
| 243038_at | RBM43 | ENSG00000184898 | 0.017653 | 0.9687316 | 7.01539 | 6.046658 | 2 | q23.3 | RNA binding motif protein 43 [Source: HGNC Symbol; Acc: 24790] |
| 212667_at | SPARC | ENSG00000113140 | 0.017553 | 2.0387778 | 9.760158 | 7.72138 | 5 | q33.1 | secreted protein, acidic, cysteine-rich (osteonectin) [Source: HGNC |
| | | | | | | | | | Symbol; Acc: 11219] |
| 209341_s_at | IKBKB | ENSG00000104365 | 0.017653 | 0.9809497 | 8.789651 | 7.808701 | 8 | p11.21 | inhibitor of kappa light polypeptide gene enhancer in B-cells, kinase beta |
| | | | | | | | | | [Source: HGNC Symbol; Acc: 5960] |
| 202336_s_at | PAM | ENSG00000145730 | 0.017653 | 1.3185154 | 9.289021 | 7.970505 | 5 | q21.1 | peptidylglycine alpha-amidating monooxygenase [Source: HGNC |
| | | | | | | | | | Symbol; Acc: 8596] |
| 212298_at | NRP1 | ENSG00000099250 | 0.018067 | 1.7793457 | 9.257538 | 7.478193 | 10 | p11.22 | neuropilin 1 [Source: HGNC Symbol; Acc: 8004] |
| 221773_at | ELK3 | ENSG00000111145 | 0.018896 | 1.6207184 | 8.175421 | 6.554703 | 12 | q23.1 | ELK3, ETS-domain protein (SRF accessory protein 2) [Source: HGNC |
| | | | | | | | | | Symbol; Acc: 3325] |
| 208816_x_at | ANXA2P2 | ENSG00000231991 | 0.018902 | 0.8785493 | 10.01166 | 9.13311 | 9 | p13.3 | annexin A2 pseudogene 2 [Source: HGNC Symbol; Acc: 539] |
| 225188_at | RAPH1 | ENSG00000173166 | 0.018902 | 2.0051889 | 7.673175 | 5.667987 | 2 | q33.2 | Ras association (RaiGDS/AF-6) and pleckstrin homology domains 1 [Source: |
| | | | | | | | | | HGNC Symbol; Acc: 14436] |
| 212779_at | KIAA1109 | ENSG00000138688 | 0.018902 | 0.9900719 | 8.187159 | 7.197087 | 4 | q27 | KIAA1109 [Source: HGNC Symbol; Acc: 26953] |
| 221748_s_at | TNS1 | ENSG00000079308 | 0.018902 | 2.0203509 | 8.86943 | 6.84908 | 2 | q35 | tensin 1 [Source: HGNC Symbol; Acc: 11973] |
| 213379_at | COQ2 | ENSG00000173085 | 0.018902 | 0.5237323 | 8.028911 | 7.505178 | 4 | q21.23 | coenzyme Q2 4-hydroxybenzoete polyprenyltransferase [Source: HGNC |
| | | | | | | | | | Symbol; Acc: 25223] |
| 211684_s_at | DYNC1I2 | ENSG00000077380 | 0.018902 | 0.8229036 | 8.910623 | 8.08772 | 2 | q31.1 | dynein, cytoplasmic 1, intermediate chain 2 [Source: HGNC Symbol; Acc: 2964] |
| 201739_at | SGK1 | ENSG00000118515 | 0.018902 | 1.4797614 | 10.75365 | 9.273888 | 6 | q23.2 | serum/glucocorticoid regulated kinase 1 [Source: HGNC Symbol; Acc: 10810] |
| 209090_s_at | SH3GLB1 | ENSG00000097033 | 0.018902 | 0.9614008 | 9.962485 | 9.001085 | 1 | p22.3 | SH3-domain GRB2-like endophilin B1 [Source: HGNC Symbol; Acc: 10833] |
| 225171_at | ARHGAP18 | ENSG00000146376 | 0.018902 | 1.5370749 | 8.677922 | 7.140848 | 6 | q22.33 | Rho GTPase activating protein 18 [Source: HGNC Symbol; Acc: 21035] |
| 204517_at | PPIC | ENSG00000168938 | 0.018902 | 1.4583399 | 7.874059 | 6.415719 | 5 | q23.2 | peptidylprolyl isomerase C (cyclophilin C) [Source: HGNC Symbol; Acc: 9256] |
| 213923_at | RAP2B | ENSG00000181467 | 0.018902 | 1.241375 | 10.18514 | 8.943765 | 3 | q25.2 | RAP2B, member of RAS oncogene family [Source: HGNC Symbol; Acc: 9862] |
| 201590_x_at | ANXA2 | ENSG00000182718 | 0.018902 | 1.2223627 | 12.76742 | 11.54506 | 15 | q22.2 | annexin A2 [Source: HGNC Symbol; Acc: 537] |
| 202202_s_at | LAMA4 | ENSG00000112769 | 0.018902 | 1.0953018 | 9.350315 | 8.255013 | 6 | q21 | laminin, alpha 4 [Source: HGNC Symbol; Acc: 6484] |
| 218718_at | PDGFC | ENSG00000145431 | 0.018921 | 2.1063846 | 7.633895 | 5.52751 | 4 | q32.1 | platelet derived growth factor C [Source: HGNC Symbol; Acc: 8801] |
| 206414_s_at | ASAP2 | ENSG00000151693 | 0.018921 | 1.4747717 | 6.713855 | 5.239084 | 2 | p25.1 | ArfGAP with SH3 domain, ankyrin repeat and PH domain 2 [Source: HGNC |
| | | | | | | | | | Symbol; Acc: 2721] |
| 212169_at | FKBP9 | ENSG00000122642 | 0.019065 | 1.1107524 | 8.896737 | 7.785985 | 7 | p14.3 | FK506 binding protein 9, 63 kDa [Source: HGNC Symbol; Acc: 3725] |
| 209348_s_at | MAF | ENSG00000178573 | 0.019065 | 2.155786 | 8.892164 | 6.736378 | 16 | q23.2 | v-maf avian musculoaponeurotic fibrosarcoma oncogene homolog [Source: HGNC |
| | | | | | | | | | Symbol; Acc: 6776] |
| 202011_at | TJP1 | ENSG00000104067 | 0.019267 | 2.0936522 | 8.408239 | 6.314587 | 15 | q13.1 | tight junction protein 1 [Source: HGNC Symbol; Acc: 11827] |
| 226823_at | PHACTR4 | ENSG00000204138 | 0.019506 | 0.7884982 | 7.613814 | 6.825316 | 1 | p35.3 | phosphatase and actin regulator 4 [Source: HGNC Symbol; Acc: 25793] |
| 201375_s_at | PPP2CB | ENSG00000104695 | 0.019506 | 0.8835548 | 10.00679 | 9.123237 | 8 | p12 | protein phosphatase 2, catalytic subunit, beta isozyme [Source: HGNC |
| | | | | | | | | | Symbol; Acc: 9300] |
| 202808_at | WBP1L | ENSG00000166272 | 0.019506 | 0.6672792 | 9.547031 | 8.879752 | 10 | q24.32 | WW domain binding protein 1-like [Source: HGNC Symbol; Acc: 23510] |
| 227911_at | ARHGAP28 | ENSG00000088756 | 0.01988 | 1.6321361 | 5.975404 | 4.343267 | 18 | p11.31 | Rho GTPase activating protein 28 [Source: HGNC Symbol; Acc: 25509] |
| 206049_at | SELP | ENSG00000174175 | 0.01988 | 1.7100903 | 7.227627 | 5.517537 | 1 | q24.2 | selectin P (granule membrane protein 140 kDa, antigen CD62) [Source: HGNC |
| | | | | | | | | | Symbol; Acc: 10721] |
| 202357_s_at | CFB | ENSG00000243649 | 0.01988 | 1.1573646 | 8.626327 | 7.468963 | 6 | p21.33 | complement factor B [Source: HGNC Symbol; Acc: 1037] |
| 217497_at | TYMP | ENSG00000025708 | 0.01988 | 0.8516144 | 8.694769 | 7.843155 | 22 | q13.33 | thymidine phosphorylase [Source: HGNC Symbol; Acc: 3148] |
| 204034_at | ETHE1 | ENSG00000105755 | 0.01988 | 0.5289265 | 8.283721 | 7.754794 | 19 | q13.31 | ethylmalonic encephalopathy 1 [Source: HGNC Symbol; Acc: 23287] |
| 210139_s_at | PMP22 | ENSG00000109099 | 0.01988 | 1.7494057 | 9.219437 | 7.470032 | 17 | p12 | peripheral myelin protein 22 [Source: HGNC Symbol; Acc: 9118] |
| 204114_at | NID2 | ENSG00000087303 | 0.01988 | 1.6354339 | 7.748453 | 6.113019 | 14 | q22.1 | nidogen 2 (osteonidogen) [Source: HGNC Symbol; Acc: 13389] |
| 200878_at | EPAS1 | ENSG00000116016 | 0.020134 | 1.6643506 | 10.74529 | 9.080936 | 2 | p21 | endothelial PAS domain protein 1 [Source: HGNC Symbol; Acc: 3374] |
| 202446_s_at | PLSCR1 | ENSG00000188313 | 0.020139 | 1.410605 | 9.74777 | 8.337165 | 3 | q24 | phospholipid scramblase 1 [Source: HGNC Symbol; Acc: 9092] |
| 221139_s_at | CSAD | ENSG00000139631 | 0.020139 | 1.1033652 | 6.32666 | 5.223294 | 12 | q13.13 | cysteine sulfinic acid decarboxylase [Source: HGNC Symbol; Acc 18966] |
| 221012_s_at | TRIM8 | ENSG00000171206 | 0.020139 | 0.9021545 | 9.733515 | 8.831361 | 10 | q24.32 | tripartite motif containing 8 [Source: HGNC Symbol; Acc: 15579] |
| 1555832_s_at | KLF6 | ENSG00000067082 | 0.020139 | 1.5050907 | 10.49276 | 8.987666 | 10 | p15.1 | Kruppel-like factor 6 [Source: HGNC Symbol; Acc: 2235] |
| 212859 x_at | MT1E | ENSG00000169715 | 0.020139 | 1.3116378 | 11.29222 | 9.98058 | 16 | q12.2 | metallothionein 1E [Source: HGNC Symbol; Acc: 7397] |
| 203729_at | EMP3 | ENSG00000142227 | 0.020691 | 1.2426119 | 10.80073 | 9.558169 | 19 | q13.33 | epithelial membrane protein 3 [Source: HGNC Symbol; Acc: 3335] |
| 209238_at | STX3 | ENSG00000166900 | 0.021317 | 1.0475218 | 7.816754 | 6.769232 | 11 | q12.1 | syntaxin 3 [Source: HGNC Symbol; Acc: 11438] |
| 228617_at | XAF1 | ENSG00000132530 | 0.02132 | 1.8591633 | 9.784426 | 7.925263 | 17 | p13.1 | XIAP associated factor 1 [Source: HGNC Symbol; Acc: 30932] |
| 204436_at | PLEKHO2 | ENSG00000241839 | 0.021661 | 0.8149093 | 9.412795 | 8.597885 | 15 | q22.31 | pleckstrin homology domain containing, family O member 2 [Source: HGNC |
| | | | | | | | | | Symbol; Acc: 30026] |
| 1555756_a_at | CLEC7A | ENSG00000172243 | 0.021739 | 1.9487017 | 9.541407 | 7.592705 | 12 | p13.2 | C-type lectin domain family 7, member A [Source: HGNC Symbol; Acc: 14558] |
| 202381_at | ADAM9 | ENSG00000168615 | 0.022252 | 1.5225689 | 9.387992 | 7.865423 | 8 | p11.22 | ADAM metallopeptidase domain 9 [Source: HGNC Symbol; Acc: 216] |
| 202291_s_at | MGP | ENSG00000111341 | 0.022612 | 2.1289603 | 11.06084 | 8.931878 | 12 | p12.3 | matrix Gla protein [Source: HGNC Symbol; Acc: 7060] |
| 213056_at | FRMD4B | ENSG00000114541 | 0.022612 | 1.7413741 | 6.023278 | 4.281904 | 3 | p14.1 | FERM domain containing 4B [Source: HGNC Symbol; Acc: 24886] |
| 218162_at | OLFML3 | ENSG00000116774 | 0.022612 | 1.5508457 | 8.32843 | 6.777584 | 1 | p13.2 | olfactomedin-like 3 [Source: HGNC Symbol; Acc: 24956] |
| 212845_at | SAMD4A | ENSG00000020577 | 0.022612 | 1.2467155 | 6.634447 | 5.387731 | 14 | q22.2 | sterile alpha motif domain containing 4A [Source: HGNC Symbol; Acc: 23023] |
| 218541_s_at | C8orf4 | ENSC00000176907 | 0.022732 | 1.9762953 | 6.693744 | 4.717449 | 8 | p11.21 | chromosome 8 open reading frame 4 [Source: HGNC Symbol; Acc: 1357] |
| 1559005_s_at | CNTLN | ENSG00000044459 | 0.022732 | 1.3908169 | 6.512895 | 5.122078 | 9 | p22.2 | centlein, centrosomal protein [Source: HGNC Symbol; Acc: 23432] |
| 218909_at | RPS6KC1 | ENSG00000136643 | 0.022732 | 0.7521578 | 8.355674 | 7.603516 | 1 | q32.3 | ribosomal protein 56 kinase, 52 kDa, polypeptide 1 [Source: HGNC |
| | | | | | | | | | Symbol; Acc: 10439] |
| 226384_at | PPAPDC1B | ENSG00000147535 | 0.022732 | 1.2111184 | 8.159726 | 6.948608 | 8 | p11.23 | phosphatidic acid phosphatase type 2 domain containing 1B [Source: HGNC |
| | | | | | | | | | Symbol; Acc: 25026] |
| 212509_s_at | MXRA7 | ENSG00000182534 | 0.022732 | 1.4674759 | 9.764658 | 8.297182 | 17 | q25.1 | matrix-remodelling associated 7 [Source: HGNC Symbol; Acc: 7541] |
| 225975_at | PCDH18 | ENSG00000189184 | 0.023159 | 1.7790238 | 7.236967 | 5.457943 | 4 | q28.3 | protocadherin 18 [Source: HGNC Symbol; Acc: 14268] |
| 201341_at | ENC1 | ENSG00000171617 | 0.023416 | 1.4443337 | 8.066608 | 6.622274 | 5 | q13.3 | ectodermal-neural cortex 1 (with BTB domain) [Source: HGNC Symbol; Acc: 3345] |
| 203324_s_at | CAV2 | ENSG00000105971 | 0.023506 | 1.952201 | 8.152024 | 6.199823 | 7 | q31.2 | caveolin 2 [Source: HGNC Symbol; Acc: 1528] |
| 212230_at | PPAP2B | ENSC00000162407 | 0.023506 | 1.8165102 | 9.071698 | 7.255187 | 1 | p32.2 | phosphatidic acid phosphatase type 2B [Source: HGNC Symbol; Acc: 9229] |
| 218632_at | HECTD3 | ENSG00000126107 | 0.023506 | 0.7369004 | 9.627648 | 8.890747 | 1 | p34.1 | HECT domain containing E3 ubiquitin protein ligase 3 [Source: HGNC |
| | | | | | | | | | Symbol; Acc: 26117] |
| 212204_at | TMEM87A | ENSG00000103978 | 0.023515 | 0.8984928 | 9.007547 | 8.109054 | 15 | q15.1 | transmembrane protein 87A [Source: HGNC Symbol; Acc: 24522] |
| 226022_at | SASH1 | ENSG00000111961 | 0.023515 | 2.0027112 | 7.610796 | 5.608085 | 6 | q24.3 | SAM and SH3 domain containing 1 [Source: HGNC Symbol; Acc: 19182] |
| 242800_at | NHS | ENSG00000188158 | 0.023677 | 1.1627115 | 6.713331 | 5.550619 | X | p22.13 | Nance-Horan syndrome (congenital cataracts and dental anomalies) [Source: HGNC |
| | | | | | | | | | Symbol; Acc: 7820] |
| 203477_at | COL15A1 | ENSG00000204291 | 0.023677 | 1.6143067 | 8.436883 | 6.822576 | 9 | q22.33 | collagen, type XV, alpha 1 [Source: HGNC Symbol; Acc: 2192] |
| 200857_s_at | NCOR1 | ENSG00000141027 | 0.023677 | 0.7091943 | 8.662953 | 7.953759 | 17 | p11.2 | nuclear receptor corepressor 1 [Source: HGNC Symbol; Acc: 7672] |
| 224996_at | ASPH | ENSG00000198363 | 0.0237 | 1.5230174 | 7.874016 | 6.350998 | 8 | q12.3 | aspartate beta-hydroxylase [Source: HGNC Symbol; Acc: 757] |
| 223562_at | PARVG | ENSG00000138964 | 0.0237 | 1.5009148 | 9.669889 | 8.168975 | 22 | q13.31 | parvin, gamma [Source: HGNC Symbol; Acc: 14654]. |
| 213353_at | ABCA5 | ENSG00000154265 | 0.0237 | 0.9962432 | 8.332971 | 7.336728 | 17 | q24.3 | ATP-binding cassette, sub-family A (ABC1), member 5 [Source: HGNC |
| | | | | | | | | | Symbol; Acc: 35] |
| 209593_s_at | TOR1B | ENSG00000136816 | 0.0238 | 0.4661131 | 7.855148 | 7.389035 | 9 | q34.11 | torsin family 1, member B (torsin B) [Source: HGNC Symbol; Acc: 11995] |
| 35626_at | SGSH | ENSG00000181523 | 0.0238 | 0.7817095 | 9.79381 | 9.0121 | 17 | g25.3 | N-sulfoglucosamine sulfohydrolase [Source: HGNC Symbol; Acc: 10818] |
| 234994_at | TMEM200A | ENSG00000164484 | 0.0238 | 1.6120474 | 6.524548 | 4.9125 | 6 | q23.1 | transmembrane protein 200A [Source: HGNC Symbol; Acc: 21075] |
| 211456_x_at | MT1P2 | NA | 0.0238 | 1.0267142 | 11.33702 | 10.3103 | NA | NA | NA |
| 203501_at | CPQ | ENSC00000104324 | 0.024112 | 0.9862063 | 7.824448 | 6.838241 | 8 | q22.1 | carboxypeptidase Q [Source: HGNC Symbol; Acc: 16910] |
| 226155_at | FAM160B1 | ENSG00000151553 | 0.024112 | 0.9499152 | 8.161808 | 7.211893 | 10 | q25.3 | family with sequence similarity 160, member B1 |
| | | | | | | | | | [Source: HGNC Symbol; Acc: 29320] |
| 1552258_at | LINC00152 | ENSG00000222041 | 0.024124 | 1.1245258 | 8.139435 | 7.014909 | 2 | p11.2 | long intergenic non-protein coding RNA 152 [Source: HGNC Symbol; Acc: 28717] |
| 238025_at | MLKL | ENSG00000168404 | 0.024866 | 1.2159905 | 8.756993 | 7.541002 | 16 | q23.1 | mixed lineage kinase domain-like [Source: HGNC Symbol; Acc: 26617] |
| 219460_s_at | TMEM127 | ENSG00000135956 | 0.024866 | 0.4546254 | 8.629221 | 8.174596 | 2 | q11.2 | transmembrane protein 127 [Source: HGNC Symbol; Acc: 26038] |
| 225522_at | AAK1 | ENSG00000115977 | 0.024866 | 1.1728016 | 8.652908 | 7.480107 | 2 | p13.3 | AP2 associated kinase 1 [Source: HGNC Symbol; Acc: 19679] |
| 217757_at | A2M | ENSG00000175899 | 0.02498 | 1.5764623 | 12.02894 | 10.45243 | 12 | p13.31 | alpha-2-macroglobulin [Source: HGNC Symbol; Acc: 7] |
| 223168_at | RHOU | ENSG00000116574 | 0.025427 | 1.5603849 | 7.215569 | 5.655184 | 1 | q42.13 | ras homolog family member U [Source: HGNC Symbol; Acc: 17794] |
| 1561615_s_at | SLC8A1 | ENSG00000183023 | 0.025808 | 1.5650362 | 8.855137 | 7.290101 | 2 | p22.1 | solute carrier family 8 (sodium/calcium exchanger), member 1 [Source: HGNC |
| | | | | | | | | | Symbol; Acc: 11068] |
| 203758_at | CTSO | ENSG00000256043 | 0.026066 | 1.1560559 | 9.311624 | 8.155568 | 4 | q32.1 | cathepsin O [Source: HGNC Symbol; Acc: 2542] |
| 226552_at | IER5L | ENSG00000133483 | 0.026467 | 0.936727 | 7.611941 | 6.675214 | 9 | q34.11 | immediate early response 5-like [Source: HGNC Symbol; Acc: 23679] |
| 202766_s_at | FBN1 | ENSG00000166147 | 0.027121 | 1.374538 | 7.745362 | 6.370824 | 15 | q21.1 | fibrillin 1 [Source: HGNC Symbol; Acc: 3603] |
| 225629_s_at | ZBTB4 | ENSG00000174282 | 0.027121 | 1.019746 | 9.543581 | 8.523835 | 17 | p13.1 | zinc finger and BTB domain containing 4 [Source: HGNC Symbol; Acc: 23847] |
| 212136_at | ATP284 | ENSG00000058668 | 0.027283 | 1.5266923 | 8.833219 | 7.306527 | 1 | q32.1 | ATPase, Ca [symbol] transporting, plasma membrane 4 |
| | | | | | | | | | [Source: HGNC Symbol; Acc: 817] |
| 1555881_s_at | LZTS2 | ENSG00000107816 | 0.027517 | 0.4402212 | 7.841075 | 7.400854 | 10 | q24.31 | leucine zipper, putative tumor suppressor 2 [Source: HGNC Symbol; Acc: 29381] |
| 57715_at | CALHM2 | ENSG00000138172 | 0.027517 | 0.6960074 | 8.099815 | 7.403808 | 10 | q24.33 | calcium homeostasis modulator 2 [Source: HGNC Symbol; Acc: 23493] |
| 226601_at | SLC30A7 | ENSG00000162695 | 0.027517 | 0.9372185 | 8.338968 | 7.401749 | 1 | p21.2 | solute carrier family 30 (zinc transporter), member 7 [Source: HGNC |
| | | | | | | | | | Symbol; Acc: 19306] |
| 217890_s_at | PARVA | ENSG00000197702 | 0.027517 | 1.5638318 | 9.160286 | 7.596454 | 11 | p15.3 | parvin, alpha [Source: HGNC Symbol; Acc: 14652] |
| 207624_s_at | RPGR | ENSG00000156313 | 0.027572 | 0.5262896 | 7.293504 | 6.767215 | X | p11.4 | retinitis pigmentosa GTPase regulator [Source: HGNC Symbol; Acc: 10295] |
| 212372_at | MYH10 | ENSG00000133026 | 0.027572 | 1.0557138 | 8.323251 | 7.267537 | 17 | p13.1 | myosin, heavy chain 10, non-muscle [Source: HGNC Symbol; Acc: 7568] |
| 226820_at | ZNF362 | ENSG00000160094 | 0.027572 | 0.8062413 | 8.807479 | 8.001238 | 1 | p35.1 | zinc finger protein 362 [Source: HGNC Symbol; Acc: 18079] |
| 202551_s_at | CRIM1 | ENSG00000150938 | 0.027572 | 1.4256957 | 8.947674 | 7.521978 | 2 | p22.3 | cysteine rich transmembrane BMP regulator 1 (chordin-like) [Source: HGNC |
| | | | | | | | | | Symbol; Acc: 2359] |
| 208030_s_at | ADD1 | ENSG00000087274 | 0.027643 | 0.584655 | 9.931156 | 9.346501 | 4 | p16.3 | adducin 1 (alpha) [Source: HGNC Symbol; Acc: 243] |
| 206618_at | IL18R1 | ENSG00000115604 | 0.027643 | 1.4815374 | 7.518533 | 6.036996 | 2 | q12.1 | interleukin 18 receptor 1 [Source: HGNC Symbol; Acc: 5988] |
| 201212_at | LGMN | ENSG00000100600 | 0.027659 | 2.066812 | 10.16982 | 8.103011 | 14 | q32.12 | legumain [Source: HGNC Symbol; Acc: 9472] |
| 208782_at | FSTL1 | ENSG00000163430 | 0.027737 | 1.3252445 | 10.08689 | 8.761649 | 3 | q13.33 | follistatin-like 1 [Source: HGNC Symbol; Acc: 3972] |
| 209191_at | TUBB6 | ENSG00000175014 | 0.027737 | 1.6575274 | 9.832241 | 8.174714 | 18 | p11.21 | tubulin, beta 6 class V [Source: HGNC Symbol; Acc: 20776] |
| 206375_s_at | SLK | ENSG00000065613 | 0.027737 | 1.0643734 | 8.057717 | 6.993344 | 10 | q24.33 | STE20-like kinase [Source: HGNC Symbol; Acc: 11088] |
| 212990_at | SYNJ1 | ENSG00000159082 | 0.027737 | 1.0442604 | 7.114954 | 6.070694 | 21 | q22.11 | synaptojanin 1 [Source: HGNC Symbol; Acc: 11503] |
| 204193_at | CHKB | ENSG00000100283 | 0.027737 | 0.8998133 | 9.518634 | 8.618821 | 22 | q13.33 | choline kinase beta [Source: HGNC Symbol; Acc: 1938] |
| 203243_s_at | PDLIM5 | ENSG00000163110 | 0.02803 | 1.1428446 | 7.842659 | 6.699815 | 4 | q22.3 | PDZ and LIM domain 5 [Source: HGNC Symbol; Acc: 17468] |
| 227554_at | MAGI2-AS3 | ENSG00000234456 | 0.02803 | 1.5835165 | 7.049678 | 5.466162 | 7 | q21.11 | MAGI2 antisense RNA 3 [Source: HGNC Symbol; Acc: 40862] |
| 224616_at | DYNC1LI2 | ENSG00000135720 | 0.02803 | 0.7957119 | 10.00718 | 9.211464 | 16 | q22.1 | dynein, cytoplasmic 1, light intermediate chain 2 [Source: HGNC |
| | | | | | | | | | Symbol; Acc: 2966] |
| 218901_at | PLSCR4 | ENSG00000114698 | 0.028186 | 1.6412008 | 5.938607 | 4.297406 | 3 | q24 | phospholipid scramblase 4 [Source: HGNC Symbol; Acc: 16497] |
| 209050_s_at | RALGDS | ENSC00000160271 | 0.028199 | 0.8301435 | 9.509798 | 8.679655 | 9 | q34.2 | ral guanine nucleotide dissociation stimulator [Source: HGNC Symbol; Acc: 9842] |
| 209472_at | CCBL2 | ENSG00000137944 | 0.028199 | 0.791466 | 9.348802 | 8.557336 | 1 | p22.2 | cysteine conjugate-beta lyase 2 [Source: HGNC Symbol; Acc: 33238] |
| 227542_at | SOCS6 | ENSG00000170677 | 0.028199 | 1.310032 | 6.680389 | 5.370357 | 18 | q22.2 | suppressor of cytokine signaling 6 [Source: HGNC Symbol; Acc: 16833] |
| 204039_at | CEBPA | ENSG00000245848 | 0.028199 | 0.9298719 | 8.572793 | 7.642921 | 19 | q13.11 | CCAA/enhancer binding protein (C/EBP), alpha [Source: HGNC |
| | | | | | | | | | Symbol; Acc: 1833] |
| 212586_at | CAST | ENSG00000153113 | 0.028199 | 0.7412945 | 9.204774 | 8.463479 | 5 | q15 | calpastatin [Source: HGNC Symbol; Acc: 1515] |
| 201280_s_at | DAB2 | ENSG00000153071 | 0.028199 | 1.7797808 | 8.603167 | 6.823386 | 5 | p13.1 | Dab, mitogen-responsive phosphoprotein, homolog 2 (Drosophila) [Source: HGNC |
| | | | | | | | | | Symbol; Acc: 2662] |
| 201599_at | OAT | ENSG00000065154 | 0.028199 | 0.8899814 | 9.474237 | 8.584256 | 10 | q26.13 | ornithine aminotransferase [Source: HGNC Symbol; Acc: 8091] |
| 225334_at | C10orf32 | ENSG00000166275 | 0.028199 | 0.7814388 | 8.253485 | 7.472047 | 10 | q24.32 | chromosome 10 open reading frame 32 [Source: HGNC Symbol; Acc: 23516] |
| 201334_s_at | ARHGEF12 | ENSG00000196914 | 0.028227 | 1.1207523 | 9.211231 | 8.090479 | 11 | q23.3 | Rho guanine nucleotide exchange factor (GEF) 12 [Source: HGNC |
| | | | | | | | | | Symbol; Acc: 14193] |
| 229041_s_at | ITGB2-AS1 | ENSG00000227039 | 0.02892 | 1.8343051 | 9.515398 | 7.681093 | 21 | q22.3 | ITGB2 antisense RNA 1 [Source: HGNC Symbol; Acc: 44304] |
| 212124_at | ZMIZ1 | ENSG00000108175 | 0.02892 | 1.4720018 | 10.25153 | 8.779525 | 10 | q22.3 | zinc finger, MIZ-type containing 1 [Source: HGNC Symbol; Acc: 16493] |
| 204863_s_at | IL6ST | ENSG00000134352 | 0.02892 | 1.1450808 | 9.161299 | 8.016219 | 5 | q11.2 | interleukin 6 signal transducer (gp130, oncostatin M receptor) [Source: HGNC |
| | | | | | | | | | Symbol; Acc: 6021] |
| 215235_at | SPTAN1 | ENSG00000197694 | 0.02892 | 0.6877124 | 10.61621 | 9.928495 | 9 | q34.11 | spectrin, alpha, non-erythrocytic 1 [Source: HGNC Symbol; Acc: 11273] |
| 212606_at | WDFY3 | ENSG00000163625 | 0.02892 | 1.6875876 | 6.840272 | 5.152685 | 4 | q21.23 | WD repeat and FYVE domain containing 3 [Source: HGNC Symbol; Acc: 20751] |
| 212601_at | ZZEF1 | ENSG00000074755 | 0.02892 | 0.4622513 | 8.027308 | 7.565057 | 17 | p13.2 | zinc finger, ZZ-type with EF-hand domain 1 [Source: HGNC Symbol; Acc: 29027] |
| 228577_x_at | ODF2L | ENSG00000122417 | 0.02892 | 0.9283355 | 7.13428 | 6.205945 | 1 | p22.3 | outer dense fiber of sperm tails 2-like [Source: HGNC Symbol; Acc: 29225] |
| 227776_at | ACER3 | ENSG00000078124 | 0.02892 | 1.1794217 | 7.521437 | 6.342016 | 11 | q13.5 | alkaline ceramidase 3 [Source: HGNC Symbol; Acc: 16066] |
| 201146_at | NFE2L2 | ENSG00000116044 | 0.02892 | 0.9705592 | 10.20237 | 9.231807 | 2 | q31.2 | nuclear factor (erythroid-derived 2)-like 2 [Source: HGNC Symbol; Acc: 7782] |
| 214807_at | LOC100509635 | NA | 0.02892 | 1.8398924 | 8.320893 | 6.481 | NA | NA | NA |
| 201185_at | HTRA1 | ENSG00000166033 | 0.02892 | 1.815072 | 9.750466 | 7.935394 | 10 | q26.13 | HtrA serine peptidase 1 [Source: HGNC Symbol; Acc: 9476] |
| 209120_at | NR2F2 | ENSG00000185551 | 0.029112 | 2.1914356 | 8.372705 | 6.181269 | 15 | q26.2 | nuclear receptor subfamily 2, group F, member 2 [Source: HGNC |
| | | | | | | | | | Symbol; Acc: 7976] |
| 226021_at | RDH10 | ENSG00000121039 | 0.029112 | 1.1810757 | 6.837597 | 5.656521 | 8 | q21.11 | retinol dehydrogenase 10 (all-trans) [Source: HGNC Symbol; Acc: 19975] |
| 224480_s_at | AGPAT9 | ENSG00000138678 | 0.029341 | 1.110371 | 4.910358 | 3.799487 | 4 | q21.23 | 1-acylglycerol-3-phosphate O-acyltransferase 9 [Source: HGNC |
| | | | | | | | | | Symbol; Acc: 28157] |
| 212077_at | CALD1 | ENSG00000122786 | 0.029341 | 1.7247901 | 10.1792 | 8.454405 | 7 | q33 | caldesmon 1 [Source: HGNC Symbol; Acc: 1441] |
| 204745_x_at | MT1G | ENSG00000125144 | 0.029341 | 1.0418968 | 10.90301 | 9.861108 | 16 | q13 | metallothionein 1G [Source: HGNC Symbol; Acc: 7399] |
| 227930_at | AGO4 | ENSG00000134698 | 0.029586 | 1.1053521 | 6.925534 | 5.820132 | 1 | p34.3 | argonaute RISC catalytic component 4 [Source: HGNC Symbol; Acc: 18424] |
| 212636_at | QKI | ENSG00000112531 | 0.029711 | 1.3783407 | 3.509471 | 7.13113 | 6 | q26 | QKI, KH domain containing, RNA binding [Source: HGNC Symbol; Acc: 21100] |
| 202609_at | EPS8 | ENSG00000151491 | 0.029743 | 2.016396 | 8.302003 | 6.285607 | 12 | p12.3 | epidermal growth factor receptor pathway substrate 8 [Source: HGNC |
| | | | | | | | | | Symbol; Acc: 3420] |
| 207791_s_at | RAB1A | ENSG00000138069 | 0.029761 | 0.8342427 | 9.926541 | 9.092298 | 2 | p14 | RAB1A, member RAS oncogene family [Source: HGNC Symbol; Acc: 9758] |
| 226377_at | NFIC | ENSG00000141905 | 0.029761 | 1.7124935 | 8.785641 | 7.660706 | 19 | p13.3 | nuclear factor I/C (CCAAT-binding transcription factor) [Source: HGNC |
| | | | | | | | | | Symbol; Acc: 7786] |
| 212607_at | AKT3 | ENSG00000117020 | 0.029761 | 1.2861363 | 8.867901 | 7.581765 | 1 | q44 | v-akt murine thymorna viral oncogene homolog 3 [Source: HGNC |
| | | | | | | | | | Symbol; Acc: 393] |
| 220122_at | MCTP1 | ENSG00000175471 | 0.029808 | 1.5794589 | 7.121595 | 5.542136 | 5 | q15 | multiple C2 domains, transmembrane 1 [Source: HGNC Symbol; Acc: 26183] |
| 225941_at | EIF4E3 | ENSG00000163412 | 0.029808 | 1.3642413 | 7.790818 | 6.426577 | 3 | p13 | eukaryotic translation initiation factor 4E family member 3 [Source: HGNC |
| | | | | | | | | | Symbol; Acc: 31837] |
| 65635_at | ENGASE | ENSG00000167280 | 0.029836 | 0.5491265 | 9.240447 | 8.69132 | 17 | q25.3 | endo-beta-N-acetylglucosaminiciase [Source: HGNC Symbol; Acc: 24622] |
| 202897_at | SIRPA | ENSG00000198053 | 0.029836 | 1.0792131 | 9.722842 | 8.643629 | 20 | p13 | signal-regulatory protein alpha [Source: HGNC Symbol; Acc: 9662] |
| 213817_at | IRAK3 | ENSG00000090376 | 0.029836 | 1.4320001 | 6.594781 | 5.162731 | 12 | q14.3 | interleukin-1 receptor-associated kinase 3 [Source: HGNC Symbol; Acc: 17020] |
| 204703_at | IFT88 | ENSG00000032742 | 0.029836 | 0.5556265 | 7.914095 | 7.358469 | 13 | q12.11 | intraflagellar transport 88 homolog (Chlamyclomonas) [Source: HGNC |
| | | | | | | | | | Symbol; Acc: 20606] |
| 201029_s_at | CD99 | ENSG00000002586 | 0.029836 | 1.1795508 | 11.79489 | 10.61534 | X | p22.33 | CD99 molecule [Source: HGNC Symbol; Acc: 7082] |
| 201105_at | LGALS1 | ENSG00000100097 | 0.029836 | 1.0679695 | 12.55396 | 11.48599 | 22 | q13.1 | lectin, galactoside-binding, soluble, 1 [Source: HGNC Symbol; Acc: 6561] |
| 228666_at | C15orf38 | ENSG00000242498 | 0.029836 | 0.9655288 | 8.632863 | 7.667335 | 15 | q26.1 | chromosome 15 open reading frame 38 [Source: HGNC Symbol; Acc: 28782] |
| 213733_at | MYO1F | ENSG00000142347 | 0.029836 | 1.2120209 | 9.047983 | 7.835962 | 19 | p13.2 | myosin IF [Source: HGNC Symbol; Acc: 7600] |
| 225162_at | SH3D19 | ENSG00000109686 | 0.029836 | 1.6977199 | 6.304496 | 4.606776 | 4 | q31.3 | SH3 domain containing 19 [Source: HGNC Symbol; Acc: 30418] |
| 221766_s_at | FAM46A | ENSG00000112773 | 0.030026 | 1.3074382 | 9.102394 | 7.794956 | 6 | q14.1 | family with sequence similarity 46, member A [Source: HGNC Symbol; Acc: 18345] |
| 235256_s_at | GALM | ENSG00000143891 | 0.030026 | 1.3208648 | 7.939207 | 6.618342 | 2 | p22.1 | galactose mutarotase (aldose 1-epimerase) [Source: HGNC Symbol; Acc: 24063] |
| 201417_at | SOX4 | ENSG00000124766 | 0.030026 | 1.0943539 | 7.314555 | 6.220202 | 6 | p22.3 | SRY (sex determining region Y)-box 4 [Source: HGNC Symbol; Acc: 11200] |
| 201963_at | ACSL1 | ENSG00000151726 | 0.030026 | 1.0262575 | 9.481285 | 8.455027 | 4 | q35.1 | acyl-CoA synthetase long-chain family member 1 [Source: HGNC |
| | | | | | | | | | Symbol; Acc: 3569] |
| 218983_at | C1RL | ENSG00000139178 | 0.030026 | 1.0535412 | 6.970689 | 5.917148 | 12 | p13.31 | complement component 1, r subcomponent-like [Source: HGNC |
| | | | | | | | | | Symbol; Acc: 21265] |
| 225442_at | DDR2 | ENSG00000162733 | 0.030026 | 1.204158 | 8.120903 | 6.916745 | 1 | q23.3 | discoidin domain receptor tyrosine kinase 2 [Source: HGNC Symbol; Acc: 2731] |
| 225128_at | KDELC2 | ENSG00000178202 | 0.030089 | 0.839869 | 7.886197 | 7.046328 | 11 | q22.3 | KDEL (Lys-Asp-Glu-Leu) containing 2 [Source: HGNC Symbol; Acc: 28496] |
| 225913_at | PEAK1 | NA | 0.030089 | 0.8774813 | 8.926048 | 8.048566 | NA | NA | NA |
| 238477_at | KIF1C | ENSG00000129250 | 0.030089 | 0.5143139 | 6.616652 | 6.102338 | 17 | p13.2 | kinesin family member 1C [Source: HGNC Symbol; Acc: 6317] |
| 1557236_at | APOL6 | ENSG00000221963 | 0.030333 | 1.3530187 | 6.588335 | 5.235316 | 22 | q12.3 | apolipoprotein L, 6 [Source: HGNC Symbol; Acc: 14870] |
| 224764_at | ARHGAP21 | ENSG00000107863 | 0.030504 | 0.7262548 | 8.341028 | 7.614773 | 10 | p12.1 | Rho GTPase activating protein 21 [Source: HGNC Symbol; Acc: 23725] |
| 204568_at | ATG14 | ENSG00000126775 | 0.030504 | 0.6825568 | 7.833225 | 7.150668 | 14 | q22.3 | autophagy related 14 [Source: HGNC Symbol; Acc: 19962] |
| 202598_at | S100A13 | ENSG00000189171 | 0.030504 | 0.9054696 | 10.25863 | 9.353158 | 1 | q21.3 | S100 calcium binding protein A13 [Source: HGNC Symbol; Acc: 10490] |
| 218315_s_at | TMEM51 | ENSG00000171729 | 0.03053 | 0.8211201 | 9.101394 | 3.280274 | 1 | p36.21 | transmembrane protein 51 [Source: HGNC Symbol; Acc: 25488] |
| 204214_s_at | RAB32 | ENSG00000118508 | 0.03053 | 0.9747791 | 8.206092 | 7.231313 | 6 | q24.3 | RAB32, member RAS oncogene family [Source: HGNC Symbol; Acc: 9772] |
| 226152_at | TTC7B | ENSG00000165914 | 0.030555 | 1.2253262 | 7.058997 | 5.833171 | 14 | q32.11 | tetratricopeptide repeat domain 7B [Source: HGNC Symbol; Acc: 19858] |
| 205590_at | RASGRP1 | ENSG00000172575 | 0.030555 | 1.8115112 | 10.05801 | 8.2465 | 15 | q14 | RAS guanyl releasing protein 1 (calcium and DAG-regulated) [Source: HGNC |
| | | | | | | | | | Symbol; Acc: 9878] |
| 1555579_s_at | PTPRM | ENSG00000173482 | 0.030555 | 1.1333216 | 7.834017 | 6.700695 | 18 | p11.23 | protein tyrosine phosphatase, receptor type, M [Source: HGNC Symbol; Acc: 9675] |
| 218486_at | KLF11 | ENSG00000172059 | 0.030555 | 1.2762837 | 7.737116 | 6.460832 | 2 | p25.1 | Kruppel-like factor 11 [Source: HGNC Symbol; Acc: 11811] |
| 204451_at | FZD1 | ENSG00000157240 | 0.030555 | 1.2800529 | 7.241574 | 5.961521 | 7 | q21.13 | frizzled family receptor 1 [Source: HGNC Symbol; Acc: 4038] |
| 226186_at | TMOD2 | ENSG00000128872 | 0.030555 | 1.3625462 | 6.784226 | 5.42168 | 15 | q21.2 | tropomodulin 2 (neuronal) [Source: HGNC Symbol; Acc: 11872] |
| 225288_at | COL27A1 | ENSG00000196739 | 0.030691 | 1.4054422 | 7.835002 | 6.429559 | 9 | q32 | collagen, type XXVII, alpha 1 [Source: HGNC Symbol; Acc: 22986] |
| 225163_at | FRMD4A | ENSG00000151474 | 0.030947 | 1.1375317 | 7.411625 | 6.274094 | 10 | p13 | FERM domain containing 4A [Source: HGNC Symbol; Acc: 25491] |
| 221541_at | CRISPLD2 | ENSG00000103196 | 0.030947 | 1.7573923 | 8.718881 | 6.961489 | 16 | q24.1 | cysteine-rich secretory protein LCCL domain containing 2 [Source: HGNC |
| | | | | | | | | | Symbol; Acc: 25248] |
| 230480_at | PIWIL4 | ENSG00000134627 | 0.030947 | 0.6575147 | 6.112214 | 5.4547 | 11 | q21 | piwi-like RNA-mediated gene silencing 4 [Source: HGNC Symbol; Acc: 18444] |
| 227444_at | ARMCX4 | ENSG00000196440 | 0.030947 | 0.97698 | 6.08167 | 5.051187 | X | q22.1 | armadillo repeat containing, X-linked 4 [Source: HGNC Symbol; Acc: 28615] |
| 218285_s_at | BDH2 | ENSG00000164039 | 0.030947 | 0.7247672 | 8.942667 | 8.2179 | 4 | q24 | 3-hydroxybutyrate dehydrogenase, type 2 [Source: HGNC Symbol; Acc: 32389] |
| 209687_at | CXCL12 | ENSG00000107562 | 0.030947 | 2.2250584 | 9.716134 | 7.491076 | 10 | q11.21 | chemokine (C-X-C motif) ligand 12 [Source: HGNC Symbol; Acc: 10672] |
| 203261_at | DCTN6 | ENSG00000104671 | 0.030947 | 0.8065653 | 10.35688 | 9.550316 | 8 | p12 | dynactin 6 [Source: HGNC Symbol; Acc: 16964] |
| 227001_at | NIPAL2 | ENSG00000104361 | 0.030947 | 1.3854199 | 8.211606 | 6.826186 | 8 | q22.2 | NIPA-like domain containing 2 [Source: HGNC Symbol; Acc: 25854] |
| 213119_at | SLC36A1 | ENSG00000123643 | 0.030947 | 0.527649 | 8.18839 | 7.660741 | 5 | q33.1 | solute carrier family 36 (proton/amino acid symporter), member 1 [Source: HGNC |
| | | | | | | | | | Symbol; Acc: 18761] |
| 201089_at | ATP6V1B2 | ENSG00000147416 | 0.030947 | 0.979669 | 10.05201 | 9.072342 | 8 | p21.3 | ATPase, H transporting, lysosomal 56/58 kDa, V1 subunit B2 [Source: HGNC |
| | | | | | | | | | Symbol; Acc: 854] |
| 228937_at | LACC1 | ENSG00000179630 | 0.030947 | 1.4347724 | 7.370869 | 6.436096 | 13 | q14.11 | laccase (multicopper oxidoreductase) domain containing 1 [Source: HGNC |
| | | | | | | | | | Symbol; Acc: 26789] |
| 219666_at | MS4A6A | ENSG00000110077 | 0.031011 | 2.1589072 | 10.17583 | 8.016976 | 11 | q12.2 | membrane-spanning 4-domains, subfamily A, member 6A [Source: HGNC |
| | | | | | | | | | Symbol; Acc: 13375] |
| 209160_at | AKR1C3 | ENSG00000196139 | 0.031011 | 1.5583408 | 6.70255 | 5.144209 | 10 | p15.1 | aldo-keto reductase family 1, member C3 [Source: HGNC Symbol; Acc: 386] |
| 203688_at | PKD2 | ENSG00000118762 | 0.031011 | 1.224664 | 8.101781 | 6.877117 | 4 | q22.1 | polycystic kidney disease 2 (autosomal dominant) [Source: HGNC |
| | | | | | | | | | Symbol; Acc: 9009] |
| 209379_s_at | CCSER2 | ENSG00000107771 | 0.031011 | 0.895303 | 6.749215 | 5.853912 | 10 | q23.1 | coiled-coil serine-rich protein 2 [Source: HGNC Symbol; Acc: 29197] |
| 202973_x_at | FAM13A | ENSG00000138640 | 0.031011 | 1.5090759 | 6.614676 | 5.1056 | 4 | q22.1 | family with sequence similarity 13, member A [Source: HGNC Symbol; Acc: 19367] |
| 222999_s_at | CCNL2 | ENSG00000221978 | 0.031011 | 0.5863072 | 9.418618 | 8.832311 | 1 | p36.33 | cyclin L2 [Source: HGNC Symbol; Acc: 20570] |
| 32811_at | MYO1C | ENSG00000197879 | 0.031088 | 0.6404769 | 9.336457 | 8.69598 | 17 | p13.3 | myosin IC [Source: HGNC Symbol; Acc: 7597] |
| 213737_x_at | GOLGA8I | ENSG00000153666 | 0.031114 | 1.2841014 | 9.220786 | 7.936684 | 15 | q11.2 | golgin A8 family, member I [Source: HGNC Symbol; Acc: 26660] |
| 202351_at | ITGAV | ENSG00000138443 | 0.031169 | 1.360409 | 9.310597 | 7.950188 | 2 | q32.1 | integrin, alpha V [Source: HGNC Symbol; Acc: 6150] |
| 226066_at | MITF | ENSG00000187098 | 0.031203 | 1.3946024 | 8.259922 | 6.86532 | 3 | p14.1 | microphthalmia-associated transcription factor [Source: HGNC Symbol; Acc: 7105] |
| 212453_at | KIAA1279 | ENSG00000198954 | 0.031375 | 0.7010336 | 7.669603 | 6.96857 | 10 | q22.1 | KIAA1279 [Source: HGNC Symbol; Acc: 23419] |
| 223276_at | SMIM3 | ENSG00000256235 | 0.031482 | 1.0571958 | 7.313512 | 6.256316 | 5 | q33.1 | small integral membrane protein 3 [Source: HGNC Symbol; Acc: 30248] |
| 63825_at | ABHD2 | ENSG00000140526 | 0.031482 | 1.0510036 | 8.787775 | 7.736771 | 15 | q26.1 | abhydrolase domain containing 2 [Source: HGNC Symbol; Acc: 18717] |
| 203817_at | GUCY1B3 | ENSG00000061938 | 0.031482 | 1.3742528 | 7.677666 | 6.303413 | 4 | q32.1 | guanylate cyclase 1, soluble, beta 3 [Source: HGNC Symbol; Acc: 4687] |
| 232090_at | DNM3OS | ENSG00000230630 | 0.031482 | 1.8766379 | 6.73819 | 4.861552 | 1 | q24.3 | DNM3 opposite strand/antisense RNA [Source: HGNC Symbol; Acc: 41228] |
| 218292_s_at | PRKAG2 | ENSG00000106617 | 0.03163 | 0.7377637 | 7.332145 | 6.594381 | 7 | q36.1 | protein kinase, AMP-activated, gamma 2 non-catalytic subunit [Source: HGNC |
| | | | | | | | | | Symbol; Acc: 9386] |
| 1558173_a_at | LUZP1 | ENSG00000169641 | 0.03163 | 0.7965467 | 9.005566 | 8.20902 | 1 | p36.12 | leucine zipper protein 1 [Source: HGNC Symbol; Acc: 14985] |
| 201438_at | COL6A3 | ENSG00000163359 | 0.03163 | 1.6730976 | 11.52 | 9.846906 | 2 | q37.3 | collagen, type VI, alpha 3 [Source: HGNC Symbol; Acc: 2213] |
| 235458_at | HAVCR2 | ENSG00000135077 | 0.03163 | 1.5671406 | 9.031225 | 7.464085 | 5 | q33.3 | hepatitis A virus cellular receptor 2 [Source: HGNC Symbol; Acc: 18437] |
| 223393_s_at | TSHZ3 | ENSG00000121297 | 0.03163 | 1.3239929 | 7.607905 | 6.283912 | 19 | q12 | teashirt zinc finger homeobox 3 [Source: HGNC Symbol; Acc: 30700] |
| 215706_x_at | ZYX | ENSG00000159840 | 0.031657 | 0.8773626 | 10.71626 | 9.838895 | 7 | q34 | zyxin [Source: HGNC Symbol; Acc: 13200] |
| 219315_s_at | TMEM204 | ENSG00000131634 | 0.0318 | 0.9451335 | 7.656937 | 6.711803 | 16 | p13.3 | transmembrane protein 204 [Source: HGNC: Symbol; Acc: 14158] |
| 201296_s_at | WSB1 | ENSG00000109046 | 0.0318 | 1.2622427 | 10.19944 | 8.937196 | 17 | q11.1 | WD repeat and SOCS box containing 1 [Source: HGNC Symbol; Acc: 19221] |
| 232231_at | RUNX2 | ENSG00000124813 | 0.0318 | 2.284395 | 8.680094 | 6.395699 | 6 | p21.1 | runt-related transcription factor 2 [Source: HGNC Symbol; Acc: 10472] |
| 226225_at | MCC | ENSG00000171444 | 0.0318 | 1.7962144 | 5.490905 | 3.69469 | 5 | q22.2 | mutated in colorectal cancers [Source: HGNC Symbol; Acc: 6935] |
| 214660_at | ITGA1 | ENSG00000213949 | 0.0318 | 0.9359119 | 4.662647 | 3.726735 | 5 | q11.2 | integrin, alpha 1 [Source: HGNC Symbol; Acc: 6134] |
| 216903_s_at | MICU1 | ENSG00000107745 | 0.0318 | 0.5179458 | 8.825263 | 8.307317 | 10 | q22.1 | mitochondrial calcium uptake 1 [Source: HGNC Symbol; Acc: 1530] |
| 215111_s_at | TSC22D1 | ENSG00000102804 | 0.0318 | 1.4896493 | 9.321974 | 7.832325 | 13 | q14.11 | TSC22 domain family, member 1 [Source: HGNC Symbol; Acc: 16826] |
| 200782_at | ANXA5 | ENSG00000164111 | 0.0318 | 0.9613548 | 10.84305 | 9.881696 | 4 | q27 | annexin A5 [Source: HGNC Svmbol; Acc: 543] |
| 226771_at | ATP8B2 | ENSG00000143515 | 0.0318 | 1.0911816 | 8.208904 | 7.117722 | 1 | q21.3 | ATPase, aminophospholipid transporter, class I, type 8B, member 2 [Source: HGNC |
| | | | | | | | | | Symbol; Acc: 13534] |
| 212185_x_at | MT2A | ENSG00000125148 | 0.0318 | 1.0146409 | 12.36689 | 11.35225 | 16 | q12.2 | metallothionein 2A [Source: HGNC Symbol; Acc: 7406] |
| 200986_at | SERPING1 | ENSG00000149131 | 0.031971 | 1.2398359 | 10.45704 | 9.217209 | 11 | q12.1 | serpin peptidase inhibitor, clade G (C1 inhibitor), member 1 [Source: HGNC |
| | | | | | | | | | Symbol; Acc: 1228] |
| 221269_s_at | SH3BGRL3 | ENSG00000142669 | 0.031971 | 0.5644103 | 11.47106 | 10.90665 | 1 | p36.11 | SH3 domain binding glutamic acid-rich protein like 3 [Source: HGNC |
| | | | | | | | | | Symbol; Acc: 15568] |
| 202081_at | IER2 | ENSG00000160888 | 0.031971 | 1.2239497 | 10.27327 | 9.049318 | 19 | p13.2 | immediate early response 2 [Source: HGNC: Symbol; Acc: 23871] |
| 206995_x_at | SCARF1 | ENSG00000074660 | 0.032439 | 0.7313947 | 6.493193 | 5.761798 | 17 | p13.3 | scavenger receptor class F, member 1 [Source: HGNC Symbol; Acc: 16820] |
| 204963_at | SSPN | ENSG00000123096 | 0.032493 | 1.9774021 | 7.971609 | 5.994207 | 12 | p12.1 | sarcospan [Source: HGNC Symbol; Acc: 11322] |
| 202192_s_at | GAS7 | ENSG00000007237 | 0.032689 | 1.0554065 | 9.145391 | 8.089984 | 17 | p13.1 | growth arrest-specific 7 [Source: HGNC Symbol; Acc: 4169] |
| 224747_at | UBE2Q2 | ENSG00000140367 | 0.032689 | 1.0044345 | 8.252427 | 7.247992 | 15 | q24.2 | ubiquitin-conjugating enzyme E2Q family member 2 [Source: HGNC |
| | | | | | | | | | Symbol; Acc: 19248] |
| 202136_at | ZMYND11 | ENSG00000015171 | 0.032689 | 1.0017022 | 9.77863 | 8.776928 | 10 | p15.3 | zinc finger, MYND-type containing 11 [Source: HGNC Symbol; Acc: 16966] |
| 209970_x_at | CASP1 | ENSG00000137752 | 0.03271 | 1.2604855 | 9.149475 | 7.888989 | 11 | q22.3 | caspase 1, apoptosis-related cysteine peptidase [Source: HGNC Symbol; Acc: 1499] |
| 234987_at | SAMHD1 | ENS600000101347 | 0.032727 | 1.4165041 | 9.200321 | 7.783817 | 20 | q11.23 | SAM domain and HD domain 1 [Source: HGNC Symbol; Acc: 15925] |
| 205173_x_at | CD58 | ENSG00000116815 | 0.032727 | 1.6045445 | 9.9085 | 8.303956 | 1 | p13.1 | CD58 molecule [Source: HGNC Symbol; Acc: 1688] |
| 229732_at | ZNF823 | ENSG00000197933 | 0.032727 | 0.5212517 | 4.981581 | 4.460329 | 19 | p13.2 | zinc finger protein 823 [Source: HGNC Symbol; Acc: 30936] |
| 37408_at | MRC2 | ENSG00000011028 | 0.032727 | 0.9769173 | 9.11946 | 8.142542 | 17 | q23.2 | mannose receptor, C type 2 [Source: HGNC Symbol; Acc: 16875] |
| 208922_s_at | NXF1 | ENSG00000162231 | 0.032727 | 0.7165582 | 10.10897 | 9.392408 | 11 | q12.3 | nuclear RNA export factor 1 [Source: HGNC Symbol; Acc: 8071] |
| 200766_at | CTSD | ENSG00000117984 | 0.032727 | 1.0316494 | 11.05559 | 10.02395 | 11 | p15.5 | cathepsin D [Source: HGNC Symbol; Acc: 2529] |
| 209129_at | TRIP6 | ENSG00000087077 | 0.032727 | 0.6143954 | 8.270125 | 7.655729 | 7 | q22.1 | thyroid hormone receptor in e tor 6 [Source: HGNC Symbol; Acc: 12311] |
| 228728_at | CPED1 | ENSG00000106034 | 0.032727 | 1.3257067 | 6.216333 | 4.890626 | 7 | q31.31 | cadharin-like and PC-esterase domain containing 1 [Source: HGNC |
| | | | | | | | | | Symbol; Acc: 26159] |
| 223028_s_at | SNX9 | ENSG00000130340 | 0.032727 | 1.5469591 | 9.600897 | 8.053938 | 6 | q25.3 | sorting nexin 9 [Source: HGNC Symbol; Acc: 14973] |
| 214464_at | CDC42BPA | ENSG00000143776 | 0.032727 | 1.5903569 | 8.711909 | 7.121553 | 1 | q42.13 | CDC42 binding protein kinase alpha (DMPK-like) |
| | | | | | | | | | [Source: HGNC Symbol; Acc: 1737] |
| 204059_s_at | ME1 | ENSG00000065833 | 0.032727 | 2.1309538 | 7.883911 | 5.752957 | 6 | q14.2 | malic enzyme 1, NADP( )-dependant, cytosolic [Source: HGNC Symbol; Acc: 6983] |
| 202481_at | DHRS3 | ENSG00000162496 | 0.032727 | 1.3557175 | 9.3739 | 8.018183 | 1 | p36.22 | dehydrogenase/reductase (SDR family) member 3 [Source: HGNC |
| | | | | | | | | | Symbol; Acc: 17693] |
| 201426_s_at | VIM | ENSG00000026025 | 0.032727 | 0.86197 | 13.16644 | 12.30447 | 10 | p13 | vimentin [Source: HGNC Symbol; Acc: 12692] |
| 214177_s_at | PBXIP1 | ENSG00000163346 | 0.032727 | 0.6439449 | 8.558974 | 7.915029 | 1 | q21.3 | pre-B-cell leukemia homeobox interacting protein 1 [Source: HGNC |
| | | | | | | | | | Symbol; Acc: 21199] |
| 218793_s_at | SCML1 | ENSG00000047634 | 0.032727 | 1.7949304 | 7.348371 | 5.553441 | X | p22.13 | sex comb on midleg-like 1 (Drosophila) [Source: HGNC Symbol; Acc: 10580] |
| 201163_s_at | IGEBP7 | ENSG00000163453 | 0.032727 | 1.2772891 | 11.02179 | 9.744497 | 4 | q12 | insulin-like growth factor binding protein 7 [Source: HGNC Symbol; Acc: 5476] |
| 225406_at | TWSG1 | ENSG00000128791 | 0.032727 | 1.0154958 | 8.499218 | 7.483723 | 18 | p11.22 | twisted gastrulation homolog 1 (Drosophila) [Source: HGNC Symbol; Acc: 12429] |
| 209071_s_at | RGS5 | ENSG00000143248 | 0.032727 | 1.9992561 | 9.334306 | 7.33505 | 1 | q23.3 | regulator of G-protein signaling 5 [Source: HGNC. Symbol; Acc: 10001] |
| 204206_at | MNT | ENSG00000070444 | 0.032727 | 0.4543166 | 8.03428 | 7.579963 | 17 | p13.3 | MNT, MAX dimerization protein [Source: HGNC Symbol; Acc: 7188] |
| 226026_at | DIRC2 | ENSG00000138463 | 0.032727 | 1.2125934 | 8.349561 | 7.136968 | 3 | q21.1 | disrupted in renal carcinoma 2 [Source: HGNC Symbol; Acc: 16623] |
| 214683_s_at | CLK1 | ENSG00000013441 | 0.032727 | 0.9215093 | 10.05348 | 9.131974 | 2 | q33.1 | CDC-like kinase 1 [Source: HGNC Symbol; Acc: 2068] |
| 218095_s_at | TMEM165 | ENSG00000131851 | 0.032727 | 0.7881717 | 10.38715 | 9.598976 | 4 | q12 | transmembrane protein 165 [Source: HGNC Symbol; Acc: 30760] |
| 219316_s_at | FLVCR2 | ENSG00000119686 | 0.032727 | 0.8558016 | 7.371184 | 6.515383 | 14 | q24.3 | feline leukemia virus subgroup C cellular receptor family, member 2 [Source: HGNC |
| | | | | | | | | | Symbol; Acc: 20105] |
| 226763_at | SESTD1 | ENSG00000187231 | 0.032727 | 1.0596477 | 9.549725 | 3.490077 | 2 | q31.2 | SEC14 and spectrin domains 1 [Source: HGNC Symbol; Acc: 18379] |
| 227361_at | HS3ST3B1 | ENSG00000125430 | 0.032727 | 1.5227184 | 7.752502 | 6.229784 | 17 | p12 | heparan sulfate (glucosamine) 3-O-sulfotransterase 3B1 [Source: HGNC |
| | | | | | | | | | Symbol; Acc: 5198] |
| 205248 _at | DOPEY2 | ENSG00000142197 | 0.032727 | 0.619808 | 7.178375 | 6.558567 | 21 | q22.12 | dopey family member 2 [Source: HGNC Symbol; Acc: 1291] |
| 211026_s_at | MGLL | ENSG00000074416 | 0.032727 | 1.3462606 | 9.704251 | 8.35799 | 3 | q21.3 | monoglyceride lipase [Source: HGNC Symbol; Acc: 17038] |
| 212224_at | ALDH1A1 | ENSG00000165092 | 0.032727 | 2.3279366 | 9.644013 | 7.316031 | 9 | q21.13 | aldehyde dehydrogenase 1 family, member A1 [Source: HGNC Symbol; Acc: 402] |
| 201215_at | PLS3 | ENSG00000102024 | 0.032727 | 1.846069 | 7.9037 | 6.057631 | X | q23 | plastin 3 [Source: HGNC Symbol; Acc: 9091] |
| 218432_at | FBXO3 | ENSG00000110429 | 0.032727 | 0.9692028 | 6.759587 | 5.7903841 | 11 | p13 | F-box protein 3 [Source: HGNC Symbol; Acc: 13532] |
| 212428_at | KIAA0368 | ENSG00000136813 | 0.032727 | 0.5027122 | 8.88096 | 8.378248 | 9 | q31.3 | KIAA0368 [Source: HGNC Symbol; Acc: 29020] |
| 203394_s_at | HES1 | ENSG00000114315 | 0.032727 | 1.1805255 | 9.074966 | 7.894441 | 3 | q29 | hairy and enhancer of split 1, (Drosophila) [Source: HGNC Symbol; Acc: 5192] |
| 235125_x_at | FAM73A | ENSG00000180488 | 0.032727 | 0.7890075 | 6.551391 | 5.762383 | 1 | p31.1 | family with sequence similarity 73, member A [Source: HGNC Symbol; Acc: 24741] |
| 224690_at | FAM210B | ENSG00000124098 | 0.032727 | 1.2185977 | 8.947456 | 7.728858 | 20 | q13.2 | family with sequence similarity 210, member B [Source: HGNC |
| | | | | | | | | | Symbol; Acc: 16102] |
| 219991_at | SLC2A9 | ENSG00000109667 | 0.032727 | 0.7452065 | 7.42728 | 6.682073 | 4 | p16.1 | solute carrier family 2 (facilitated glucose transporter), member 9 [Source: HGNC |
| | | | | | | | | | Symbol; Acc: 13446] |
| 214560_at | FPR3 | ENSG00000187474 | 0.032727 | 1.5525474 | 10.11159 | 8.55904 | 19 | q13.41 | formyl peptide receptor 3 [Source: HGNC Symbol; Acc: 3828] |
| 210946_at | PPAP2A | ENSG00000067113 | 0.032727 | 0.8694264 | 8.48212 | 7.612694 | 5 | q11.2 | phosphatidic acid phosphatase type 2A [Source: HGNC Symbol; Acc: 9228] |
| 210817_s_at | CALCOCO2 | ENSG00000136436 | 0.032727 | 0.9010721 | 10.04862 | 9.147547 | 17 | q21.32 | calcium binding and coiled-coil domain 2 [Source: HGNC Symbol; Acc: 29912] |
| 208636_at | ACTN1 | ENSG00000072110 | 0.032727 | 1.4921308 | 9.059967 | 7.567836 | 14 | q24.1 | actinin, alpha 1 [Source: HGNC Symbol; Acc: 163] |
| 1561226_at | XCR1 | ENSG00000173578 | 0.032727 | 0.9280383 | 4.980618 | 4.05258 | 3 | p21.31 | chemokine (C motif) receptor 1 [Source: HGNC Symbol; Acc: 1625] |
| 229256_at | PGM2L1 | ENSG00000165434 | 0.032727 | 0.9502539 | 6.806744 | 5.85649 | 11 | q13.4 | phosphoglucomutase 2-like 1 [Source: HGNC Symbol; Acc: 20898] |
| 228131_at | ERCC1 | ENSG00000012061 | 0.032727 | 0.6986806 | 8.644378 | 7.945697 | 19 | q13.32 | excision repair cross-complementing rodent repair deficiency, complementation |
| | | | | | | | | | group 1 (includes overlapping antisense sequence) [Source: HGNC |
| | | | | | | | | | Symbol; Acc: 3433] |
| 209310_s_at | CASP4 | ENSG00000196954 | 0.032736 | 0.9103894 | 10.59028 | 9.679891 | 11 | q22.3 | caspase 4, apoptosis-related cysteine peptidase [Source: HGNC Symbol; Acc: 1505] |
| 218729_at | LXN | ENSG00000079257 | 0.032736 | 1.3734357 | 9.362978 | 7.989542 | 3 | q25.32 | latexin [Source: HGNC Symbol; Acc: 13347] |
| 227274_at | SYNJ2BP | ENSG00000213463 | 0.032835 | 0.9456553 | 8.969654 | 8.023999 | 14 | q24.2 | synaptojanin 2 binding protein [Source: HGNC Symbol; Acc: 18955] |
| 219147_s_at | NMRK1 | ENSG00000106733 | 0.032835 | 1.031303 | 8.134786 | 7.103483 | 9 | q21.13 | nicotinamide riboside kinase 1 [Source: HGNC Symbol; Acc: 26057] |
| 205083_at | AOX1 | ENSG00000138356 | 0.032835 | 1.3447575 | 6.346469 | 5.001711 | 2 | q33.1 | aldehyde oxidase 1 [Source: HGNC Symbol; Acc: 553] |
| 209109_s_at | TSPAN6 | ENSG00000000003 | 0.03287 | 1.3163026 | 7.197532 | 5.88123 | X | q22.1 | tetraspanin 6 [Source: HGNC Symbol; Acc: 11858] |
| 225303_at | KIRREL | ENSG00000183853 | 0.03287 | 1.2844668 | 7.658036 | 6.373569 | 1 | q23.1 | kin of IRRE like (Drosophila) [Source: HGNC Symbol; Acc: 15734] |
| 226185_at | MRAS | ENSG00000158186 | 0.03287 | 1.0377007 | 7.834459 | 6.796758 | 3 | q22.3 | muscle RAS oncogene homolog [Source: HGNC Symbol; Acc: 7227] |
| 203716_s_at | DPP4 | ENSG00000197635 | 0.03287 | 1.5099205 | 8.788772 | 7.278851 | 2 | q24.2 | dipeptidyl-peptidase 4 [Source: HGNC Symbol; Acc: 3009] |
| 204342_at | SLC25A24 | ENSG00000085491 | 0.03287 | 0.9998009 | 8.623813 | 7.624012 | 1 | p13.3 | solute carrier family 25 (mitochondrial carrier; phosphate carrier), member 24 |
| | | | | | | | | | [Source: HGNC Symbol; Acc: 20662] |
| 202948_at | IL1R1 | ENSG00000115594 | 0.033188 | 1.7497853 | 8.584053 | 6.834267 | 2 | q11.2 | interleukin 1 receptor, type I [Source: HGNC Symbol; Acc: 5993] |
| 212511_at | PICALM | ENSG00000073921 | 0.033188 | 0.7422533 | 6.915631 | 6.173378 | 11 | q14.2 | phosphatidylinositol binding clathrin assembly protein [Source: HGNC |
| | | | | | | | | | Symbol; Acc: 15514] |
| 214429_at | MTMR6 | ENSG00000139505 | 0.033466 | 0.7697304 | 8.954555 | 8.184824 | 13 | q12.13 | myotubularin related protein 6 [Source: HGNC Symbol; Acc: 745.3] |
| 214021_x_at | ITGB5 | ENSG00000082781 | 0.033488 | 1.4359772 | 6.631669 | 5.195692 | 3 | q21.2 | integrin, beta 5 [Source: HGNC Symbol; Acc: 6160] |
| 227624_at | TET2 | ENSG00000168769 | 0.033488 | 1.0769098 | 7.558846 | 6.481937 | 4 | q24 | tet methylcytosine dioxygenase 2 [Source: HGNC Symbol; Acc: 25941] |
| 225093_at | UTRN | ENSG00000152818 | 0.033488 | 1.5657154 | 10.04129 | 8.415572 | 6 | q24.2 | utrophin [Source: HGNC Symbol; Acc: 12635] |
| 227379_at | MBOAT1 | ENSG00000172197 | 0.033637 | 1.2267144 | 7.197727 | 5.971012 | 6 | p22.3 | membrane bound O-acyltransferase domain containing 1 [Source: HGNC |
| | | | | | | | | | Symbol; Acc: 21579] |
| 213139_at | SNAI2 | ENSG00000019549 | 0.033801 | 1.5495817 | 6.95332 | 5.403738 | 8 | q11.21 | snail family zinc finger 2 [Source: HGNC Symbol; Acc: 11094] |
| 212099_at | RHOB | ENSG00000143878 | 0.033801 | 1.5631977 | 9.851473 | 8.288275 | 2 | p24.1 | ras homolog family member B [Source: HGNC Symbol; Acc: 668] |
| 204894_s_at | AOC3 | ENSG00000131471 | 0.033801 | 1.4887152 | 6.950874 | 5.462159 | 17 | q21.31 | amine oxidase, copper containing 3 [Source: HGNC: Symbol; Acc: 550] |
| 219432_at | EVC | ENSG00000072840 | 0.033801 | 0.9734954 | 7.657381 | 6.683835 | 4 | p16.2 | Ellis van Creveld syndrome [Source: HGNC Symbol; Acc: 3497] |
| 55065_at | MARK4 | ENSG00000007047 | 0.033801 | 0.4501318 | 7.655069 | 7.204937 | 19 | q13.32 | MAP/microtubule affinity-regulating kinase 4 [Source: HGNC Symbol; Acc: 13538] |
| 226353_at | SPPL2A | ENSG00000138600 | 0.034001 | 1.0860363 | 9.824238 | 8.738201 | 15 | q21.2 | signal peptide peptidase like 2A [Source: HGNC Symbol; Acc: 30227] |
| 216598_s_at | CCL2 | ENSG00000108691 | 0.034001 | 1.5741003 | 11.80437 | 10.23027 | 17 | q12 | chemokine (C-C motif) ligand 2 [Source: HGNC Symbol; Acc: 10618] |
| 226568_at | FAM102B | ENSG00000162636 | 0.034001 | 1.1949347 | 7.320321 | 6.125386 | 1 | p13.3 | family with sequence similarity 102, member B [Source: HGNC Symbol; Acc: 27637] |
| 218764_at | PRKCH | ENSG00000027075 | 0.034091 | 1.3242248 | 8.842467 | 7.53.8243 | 14 | q23.1 | protein kinase C, eta [Source: HGNC Symbol; Acc: 9403] |
| 225984_at | PRKAA1 | ENSG00000132356 | 0.034118 | 0.7616255 | 6.863442 | 6.101816 | 5 | p13.1 | protein kinase, AMP-activated, alpha 1 catalytic. subunit [Source: HGNC |
| | | | | | | | | | Symbol; Acc: 9376] |
| 201542_at | SAR1A | ENSG00000079332 | 0.034118 | 0.7356457 | 9.772431 | 9.036785 | 10 | q22.1 | SAR1 homolog A (S. cerevisiae) [Source: HGNC Symbol; Acc: 10534] |
| 203455_s_at | SAT1 | ENSG00000130066 | 0.034118 | 1.31652 | 11.13299 | 9.816473 | X | p22.11 | spermidine/spermine N1-acetyltransferase 1 [Source: HGNC Symbol; Acc: 10540] |
| 225922_at | FNIP2 | ENSG00000052795 | 0.034118 | 1.5195275 | 8.166224 | 6.646697 | 4 | q32.1 | folliculin interacting protein 2 [Source: HGNC Symbol; Acc: 29280] |
| 218665_at | FZD4 | ENSG00000174804 | 0.034118 | 1.0211632 | 7.416231 | 6.395068 | 11 | q14.2 | frizzled family receptor 4 [Source: HGNC Symbol; Acc: 4042] |
| 221666_s_at | PYCARD | ENSG00000103490 | 0.034191 | 0.6061082 | 9.397236 | 8.791128 | 16 | p11.2 | PYD and CARD domain containing [Source: HGNC Symbol; Acc: 16608] |
| 201058_s_at | MYL9 | ENSG00000101335 | 0.034191 | 1.2676991 | 10.37627 | 9.108573 | 20 | q11.23 | myosin, light chain 9, regulatory [Source: HGNC Symbol; Acc: 15754] |
| 203397_s_at | GALNT3 | ENSG00000115339 | 0.034191 | 1.3066403 | 5.451049 | 4.144409 | 2 | q24.3 | UDP-N-acetyl-alpha-D-galactosamine: polypeptide N- |
| | | | | | | | | | acetylgalactosaminyltransferase 3 (GalNAc-T3) [Source: HGNC Symbol; Acc: 4125] |
| 222453_at | CYBRD1 | ENSG00000071967 | 0.034191 | 1.3157278 | 9.001392 | 7.685664 | 2 | q31.1 | cytochrome b reductase 1 [Source: HGNC Symbol; Acc: 20797] |
| 208146_s_at | CPVL | ENSG00000106066 | 0.034191 | 1.9432647 | 9.534937 | 7.591672 | 7 | p14.3 | carboxypeptidase, vitellogenic-like [Source: HGNC Symbol; Acc: 14399] |
| 227334_at | USP54 | ENSG00000166348 | 0.034191 | 0.5950048 | 7.632457 | 7.037452 | 10 | q22.2 | ubiquitin specific peptidase 54 [Source: HGNC Symbol; Acc: 23513] |
| 226534_at | KITLG | ENSG00000049130 | 0.034191 | 1.6871203 | 6.504808 | 4.817688 | 12 | q21.32 | KIT ligand [Source: HGNC Symbol; Acc: 6343] |
| 229120_s_at | CDC42SE1 | ENSG00000197522 | 0.034191 | 1.0261208 | 9.039742 | 8.013621 | 1 | q21.3 | CDC42 small effector 1 [Source: HGNC Symbol; Acc: 17719] |
| 225066_at | PPP2R2D | ENSG00000175470 | 0.034191 | 0.4100232 | 6.515125 | 6.105102 | 10 | q26.3 | protein phosphatase 2, regulatory subunit B, delta [Source: HGNC |
| | | | | | | | | | Symbol; Acc: 23732] |
| 211977_at | GPR107 | ENSG00000148358 | 0.034191 | 0.4150635 | 7.32333 | 6.908267 | 9 | q34.11 | G protein-coupled receptor 107 [Source: HGNC Symbol; Acc: 17830) |
| 217967_s_at | FAM129A | ENSG00000135842 | 0.034191 | 1.6316176 | 9.361852 | 7.730234 | 1 | q25.3 | family with sequence similarity 129, member A [Source: HGNC Symbol; Acc: 16784] |
| 207705_s_at | NINL | ENSG00000101004 | 0.034191 | 0.4580325 | 7.469272 | 7.011239 | 20 | p11.21 | ninein-like [Source: HGNC Symbol; Acc: 29163] |
| 230029_x_at | UBR3 | ENSG00000144357 | 0.034212 | 0.7132327 | 7.406814 | 6.693581 | 2 | q31.1 | ubiquitin protein ligase E3 component n-recognin 3 (putative) [Source: HGNC |
| | | | | | | | | | Symbol; Acc: 30467] |
| 227236_at | TSPAN2 | ENSG00000134198 | 0.03424 | 1.1605259 | 6.827258 | 5.666732 | 1 | p13.2 | tetraspanin 2 [Source: HGNC Symbol; Acc: 20659] |
| 202510_s_at | TNFAIP2 | ENSG00000185215 | 0.03424 | 1.6113115 | 10.98695 | 9.375641 | 14 | q32.32 | tumor necrosis factor, alpha-induced protein 2 [Source: HGNC Symbol; Acc: 11895] |
| 202450_s_at | CTSK | ENSG00000143387 | 0.034334 | 2.0587683 | 10.3433 | 8.284534 | 1 | q21.3 | cathepsin K [Source: HGNC Symbol; Acc: 2536] |
| 226395_at | HOOK3 | ENSG00000168172 | 0.034361 | 0.9073842 | 9.099733 | 8.192349 | 8 | p11.21 | hook homolog 3 (Drosophila) [Source: HGNC Symbol; Acc: 23576] |
| 219403_s_at | HPSE | ENSG00000173083 | 0.034374 | 1.2733775 | 7.073411 | 5.800034 | 4 | q21.23 | heparanase [Source: HGNC Symbol; Acc: 5164] |
| 227623_at | CACNA2D1 | ENSG00000153956 | 0.034401 | 1.5093742 | 5.523191 | 4.013817 | 7 | q21.11 | calcium channel, voltage-dependent, alpha 2/delta subunit 1 [Source: HGNC |
| | | | | | | | | | Symbol; Acc: 1399] |
| 202239_at | PARP4 | ENSG00000102699 | 0.034401 | 0.9202135 | 9.568361 | 8.648148 | 13 | q12.12 | poly (ADP-ribose) polymerase family, member 4 [Source: HGNC Symbol; Acc: 271] |
| 204646_at | DPYD | ENSG00000188641 | 0.034481 | 1.7165381 | 8.184742 | 6.468204 | 1 | p21.3 | dihydropyrimidine dehydrogenase [Source: HGNC Symbol; Acc: 3012] |
| 209335_at | DCN | ENSG00000011465 | 0.034607 | 2.0562205 | 8.106748 | 6.050528 | 12 | q21.33 | decorin [Source: HGNC Symbol; Acc: 2705] |
| 202341_s_at | TRIM2 | ENSG00000109654 | 0.034607 | 1.1549983 | 7.682678 | 6.52768 | 4 | q31.3 | tripartite motif containing 2 [Source: HGNC Symbol; Acc: 15974] |
| 203386_at | TBC1D4 | ENSG00000136111 | 0.034612 | 1.5986356 | 9.927925 | 8.329289 | 13 | q22.2 | TBC1 domain family, member [Source: HGNC Symbol; Acc: 19165] |
| 202465_at | PCOLCE | ENSG00000106333 | 0.034687 | 1.1107939 | 9.858341 | 8.747547 | 7 | q22.1 | procollagen C-endopeptidase enhancer [Source: HGNC Symbol; Acc: 8738] |
| 218501_at | ARHGEF3 | ENSG00000153947 | 0.03471 | 1.211901 | 9.645779 | 8.433878 | 3 | p14.3 | Rho guanine nucleotide exchange factor (GEF) 3 [Source: HGNC Symbol; Acc: 683] |
| 203935_at | ACVR1 | ENSG00000115170 | 0.03471 | 0.9297485 | 7.59294 | 6.663191 | 2 | q24.1 | activin A receptor, type I [Source: HGNC Symbol; Acc: 171] |
| 227726_at | RNF166 | ENSG00000158717 | 0.034778 | 0.7652554 | 6.490242 | 5.724987 | 16 | q24.3 | ring finger protein 166 [Source: HGNC Symbol; Acc: 23856] |
| 225351_at | FAM45A | ENSG00000119979 | 0.034778 | 0.712687 | 7.985558 | 7.272871 | 10 | q26.11 | family with sequence similarity 45, member A [Source: HGNC Symbol; Acc: 31793] |
| 209967_s_at | CREM | ENSG00000095794 | 0.034778 | 1.4987082 | 8.008163 | 6.509455 | 10 | p11.21 | cAMP responsive element modulator [Source: HGNC Symbol; Acc: 2352] |
| 217767_at | C3 | ENSG00000125730 | 0.034902 | 1.630303 | 11.06256 | 9.452253 | 19 | p13.3 | complement component 3 [Source: HGNC Symbol; Acc: 1318] |
| 202377_at | LEPROT | ENSG00000213625 | 0.034902 | 0.9587583 | 8.927718 | 7.96896 | 1 | p31.3 | leptin receptor overlapping transcript [Source: HGNC Symbol; Acc: 29477] |
| 212067_s_at | C1R | ENSG00000159403 | 0.034902 | 1.0958838 | 10.9773 | 9.881414 | 12 | p13.31 | complement component 1, r subcomponent [Source: HGNC Symbol; Acc: 1246] |
| 206461_x_at | MT1H | ENSG00000205358 | 0.034907 | 0.9374798 | 10.9411 | 10.00362 | 16 | q13 | metallothionein 1H [Source: HGNC Symbol; Acc: 7400] |
| 212651_at | RHOBTB1 | ENSG00000072422 | 0.03492 | 1.2780518 | 5.303259 | 4.025208 | 10 | q21.2 | Rho-related BTB domain containing 1 [Source: HGNC Symbol; Acc: 18738] |
| 213077_at | YTHDC2 | ENSG00000047188 | 0.03492 | 1.0822747 | 7.160913 | 6.078638 | 5 | q22.2 | YTH domain containing 2 [Source: HGNC Symbol; Acc: 24721] |
| 238617_at | KIF26B | ENSG00000162849 | 0.03492 | 1.8167267 | 7.548087 | 5.73136 | 1 | q44 | kinesin family member 26B [Source: HGNC Symbol; Acc: 25484] |
| 201540_at | FHL1 | ENSG00000022267 | 0.03492 | 2.0909145 | 8.481468 | 6.390554 | X | q26.3 | four and a half LIM domains 1 [Source: HGNC Symbol; Acc: 3702] |
| 227325_at | PRR24 | ENSG00000257704 | 0.03492 | 0.5900983 | 7.823938 | 7.23384 | 19 | q13.32 | proline rich 24 [Source: HGNC Symbol; Acc: 27406] |
| 208093_s_at | NDEL1 | ENSG00000166579 | 0.034947 | 0.7410348 | 8.576081 | 7.835046 | 17 | p13.1 | nudE nuclear distribution E homolog (A. nidulans)-like 1 [Source: HGNC |
| | | | | | | | | | Symbol; Acc: 17620] |
| 202006_at | PTPN12 | ENSG00000127947 | 0.035089 | 1.0182628 | 9.540274 | 8.522011 | 7 | q11.23 | protein tyrosine phosphatase, non-receptor type 12 [Source: HGNC |
| | | | | | | | | | Symbol; Acc: 9645] |
| 209216_at | WDR45 | ENSG00000196998 | 0.035089 | 0.696795 | 8.826888 | 8.130093 | X | q11.23 | WD repeat domain 45 [Source: HGNC Symbol; Acc: 28912] |
| 225782_at | MSRB3 | ENSG00000174099 | 0.035089 | 1.4443755 | 6.51473.6 | 5.07034 | 12 | q14.3 | methionine sulfoxide reductase B3 [Source: HGNC Symbol; Acc: 27375] |
| 200602_at | APP | ENSG00000142192 | 0.035089 | 1.32593 | 9.055535 | 7.729605 | 21 | q21.3 | amyloid beta (A4) precursor protein [Source: HGNC Symbol; Acc: 620] |
| 226939_at | CPEB2 | ENSG00000137449 | 0.035264 | 1.2264987 | 7.611624 | 6.385126 | 4 | p15.33 | cytoplasmic polyadenylation element binding protein 2 [Source: HGNC |
| | | | | | | | | | Symbol; Acc: 21745] |
| 218986_5_at | DDX60 | ENSG00000137628 | 0.035316 | 1.4860214 | 8.318531 | 6.83251 | 4 | q32.3 | DEAD (Asp-Glu-Ala-Asp) box polypeptide 60 [Source: HGNC Symbol; Acc: 25942] |
| 203139_at | DAPK1 | ENSG00000196730 | 0.035316 | 1.7079489 | 8.912548 | 7.204599 | 9 | q21.33 | death-associated protein kinase 1 [Source: HGNC Symbol; Acc: 2674] |
| 213764_s_at | MFAP5 | ENSG00000197614 | 0.035435 | 1.2979822 | 5.790529 | 4.492547 | 12 | p13.31 | microfibrillar associated protein 5 [Source: HGNC Symbol; Acc: 29673] |
| 202565_s_at | SVIL | ENSG00000197321 | 0.035639 | 1.3890512 | 8.635445 | 7.246394 | 10 | p11.23 | supervillin [Source: HGNC Symbol; Acc: 11480] |
| 210968_s_at | RTN4 | ENSG00000115310 | 0.03577 | 0.8295931 | 10.69653 | 9.866939 | 2 | p16.1 | reticulon 4 [Source: HGNC Symbol; Acc: 14085] |
| 225562_at | RASA3 | ENSG00000185989 | 0.03578 | 0.9721275 | 9.062085 | 8.089957 | 13 | q34 | RAS p21 protein activator 3 [Source: HGNC Symbol; Acc: 20331] |
| 218454_at | PLBD1 | ENSG00000121316 | 0.035786 | 1.4419824 | 10.25392 | 8.811935 | 12 | p13.1 | phospholipase B domain containing 1 [Source: HGNC Symbol; Acc: 26215] |
| 209467_s_at | MKNK1 | ENSG00000079277 | 0.035868 | 0.7832944 | 8.294847 | 7.511553 | 1 | p33 | MAP kinase interacting serine/threonine kinase 1 [Source: HGNC Symbol; Acc: 7110] |
| 226869_at | MEGF6 | ENSG00000162591 | 0.03595 | 1.2650434 | 7.859101 | 6.594058 | 1 | p36.32 | multiple EGF-like-domains 6 [Source: HGNC Symbol; Acc: 3232] |
| 221569_at | AHI1 | ENSG00000135541 | 0.03595 | 1.0761406 | 7.793652 | 6.717512 | 6 | q23.3 | Abelson helper integration site 1 [Source: HGNC Symbol; Acc: 21575] |
| 1569157_s_at | ZNF846 | ENSG00000196605 | 0.03595 | 0.7746233 | 6.057018 | 5.282395 | 19 | p13.2 | zinc finger protein 846 [Source: HGNC Symbol; Acc: 27260] |
| 218132_s_at | TSEN34 | ENSG00000170892 | 0.03595 | 0.4388666 | 8.237399 | 7.798532 | 19 | q13.42 | tRNA splicing endonuclease 34 homolog (S. cerevisiae) [Source: HGNC |
| | | | | | | | | | Symbol; Acc: 15506] |
| 201591_s_at | NISCH | ENSG00000010322 | 0.036284 | 0.4056482 | 8.996513 | 8.590865 | 3 | p21.1 | nischarin [Source: HGNC Symbol; Acc: 18006] |
| 227098_at | DUSP18 | ENSG00000167065 | 0.036417 | 0.4821536 | 7.203923 | 6.721759 | 22 | q12.2 | dual specificity phosphatase 18 [Source: HGNC Symbol; Acc: 18484] |
| 203910_at | ARHGAP29 | ENSG00000137962 | 0.036417 | 1.5131699 | 7.336341 | 5.823171 | 1 | p21.3 | Rho GTPase activating protein 29 [Source: HGNC Symbol; Acc: 30207] |
| 1555847_a_at | LOC2844540 | NA | 0.036417 | 0.9290134 | 9.021958 | 8.092945 | NA | NA | NA |
| 241353_s_at | LOC1005075 | NA | 0.036417 | 0.563124 | 6.938192 | 6.375068 | NA | NA | NA |
| | 07 | | | | | | | | |
| 202225_at | CRK | ENSG00000167193 | 0.036417 | 0.7660484 | 9.380488 | 8.614439 | 17 | p13.3 | v-crk avian sarcoma virus CT10 oncogene homolog [Source: HGNC |
| | | | | | | | | | Symbol; Acc: 2362] |
| 212915_at | PDZRN3 | ENSG00000121440 | 0.036491 | 1.3125793 | 6.719045 | 5.406466 | 3 | p13 | PDZ domain containing ring finger 3 [Source: HGNC Symbol; Acc: 17704] |
| 202920_at | ANK2 | ENSG00000145362 | 0.036491 | 1.4633941 | 8.124628 | 6.661234 | 4 | q25 | ankyrin 2, neuronal [Source: HGNC Symbol; Acc: 493] |
| 235391_at | FAM92A1 | ENSG00000188343 | 0.036548 | 0.6211362 | 7.223439 | 6.602303 | 8 | q22.1 | family with sequence similarity 92, member A1 [Source: HGNC Symbol; Acc: 30452] |
| 224906_at | ANO6 | ENSG00000177119 | 0.036675 | 0.8771124 | 8.664683 | 7.787571 | 12 | q12 | anoctamin 6 [Source: HGNC Symbol; Acc: 25240] |
| 227087_at | INPP4A | ENSG00000040933 | 0.036675 | 1.0267894 | 8.031874 | 7.005085 | 2 | q11.2 | inositol polyphosphate-4-phosphatase, type I, 107 kDa [Source: HGNC |
| | | | | | | | | | Symbol; Acc: 6074] |
| 225931_s_at | RNF213 | ENSG00000173821 | 0.036802 | 1.1072523 | 9.749178 | 8.641926 | 17 | q25.3 | ring finger protein 213 [Source: HGNC Symbol; Acc: 14539] |
| 212690_at | DDHD2 | ENSG00000085788 | 0.036831 | 0.8783562 | 9.176867 | 8.298511 | 8 | p11.23 | DDHD domain containing 2 [Source: HGNC Symbol; Acc: 29106] |
| 209003_at | SLC25A11 | ENSG00000108528 | 0.036891 | 0.4377232 | 7.954667 | 7.516944 | 17 | p13.2 | solute carrier family 25 (mitochondrial carrier; oxoglutarate carrier), member 11 |
| | | | | | | | | | [Source: HGNC Symbol; Acc: 10981] |
| 202206_at | ARL4C | ENSG00000188042 | 0.036891 | 1.3898051 | 9.321387 | 7.931582 | 2 | q37.1 | ADP-ribosylation factor-like 4C [Source: HGNC Symbol; Acc: 698] |
| 232000_at | ITC39B | ENSG00000155158 | 0.037068 | 1.3565126 | 4.978733 | 3.62222 | 9 | p22.3 | tetrathcopeptide repeat domain 39B [Source: HGNC Symbol; Acc: 23704] |
| 225386_s_at | HNRPLL | NA | 0.037068 | 1.4593954 | 9.121602 | 7.662206 | NA | NA | NA |
| 2264092_at | TBC1D20 | ENSG00000125875 | 0.037068 | 0.5055858 | 8.965324 | 3.459738 | 20 | p13 | TBC1 domain family, member 20 [Source: HGNC Symbol; Acc: 16133] |
| 204066_s_at | AGAP1 | ENSG00000157985 | 0.037088 | 1.0789416 | 6.07508 | 4.996139 | 2 | q37.2 | ArfGAP with GTPase domain, ankyrin repeat and PH domain 1 [Source: HGNC |
| | | | | | | | | | Symbol; Acc: 16922] |
| 212441_at | KIAA0232 | ENSG00000170871 | 0.037088 | 0.5902105 | 8.606786 | 8.016575 | 4 | p16.1 | KIAA0232 [Source: HGNC Symbol; Acc: 28992] |
| 225726_s_at | PLEKHH1 | ENSG00000054690 | 0.037088 | 0.9526951 | 5.964958 | 5.012263 | 14 | q24.1 | pleckstrin homology domain containing, family H (with MyTH4 domain) member 1 |
| | | | | | | | | | [Source: HGNC Symbol; Acc: 17733] |
| 232094_at | KATNBL1 | ENSG00000134152 | 0.037088 | 0.5971703 | 7.264443 | 6.667273 | 15 | q14 | katenin p80 subunit B-like 1 [Source: HGNC Symbol; Acc: 26199] |
| 225604_s_at | GLIPR2 | ENSG00000122694 | 0.037324 | 0.8539107 | 7.844159 | 6.990248 | 9 | p13.3 | GLI pathogenesis-related 2 [Source: HGNC Symbol; Acc: 18007] |
| 205225_at | ESR1 | ENSG00000091831 | 0.037324 | 1.3824683 | 6.467921 | 5.085453 | 6 | q25.1 | estrogen receptor [Source: HGNC Symbol; Acc: 3467] |
| 218838_s_at | TTC31 | ENSG00000115282 | 0.037324 | 0.6726502 | 8.524158 | 7.851508 | 2 | p13.1 | tetra tricopeptirle repeat domain 31 [Source: HGNC Symbol; Acc: 25759] |
| 227961_at | CTSB | ENSG00000164733 | 0.037324 | 1.3381396 | 10.24117 | 8.903035 | 8 | p23.1 | cathepsin B [Source: HGNC Symbol; Acc: 2527] |
| 1558041_a_at | KIAA0895L | ENSG00000196123 | 0.037413 | 0.8767425 | 7.925107 | 7.048364 | 16 | q22.1 | KIAA0395-like [Source: HGNC Symbol; Acc: 34408] |
| 209540_at | IGF1 | ENSG00000017427 | 0.037413 | 1.5225095 | 8.256633 | 6.734124 | 12 | q23.2 | insulin-like growth factor 1 (somatomedin C) [Source: HGNC Symbol; Acc: 5464] |
| 203651_at | ZFYVE16 | ENSG00000039319 | 0.037413 | 1.0251416 | 8.527484 | 7.502343 | 5 | q14.1 | zinc finger, FYVE domain containing 16 [Source: HGNC Symbol; Acc: 20756] |
| 224900_at | ANKFY1 | ENSG00000185722 | 0.037546 | 0.532953 | 8.58147 | 8.048518 | 17 | p13.2 | ankyrin repeat and FYVE domain containing 1 [Source: HGNC Symbol; Acc: 20763] |
| 223441_at | SLC17A5 | ENSG00000119899 | 0.037627 | 0.645006 | 6.824616 | 6.17961 | 6 | q13 | solute carrier family 17 (anion/sugar transporter), member 5 [Source: HGNC |
| | | | | | | | | | Symbol; Acc: 10933] |
| 204294_at | AMT | ENSG00000145020 | 0.037627 | 0.3994342 | 8.335214 | 7.935779 | 3 | p21.31 | aminomethyltransferase [Source: HGNC Symbol; Acc: 473] |
| 204040_at | RNF144A | ENSG00000151692 | 0.037682 | 1.4813478 | 6.933004 | 5.451656 | 2 | p25.2 | ring finger protein 144A [Source: HGNC: Symbol; Acc: 20457] |
| 225272_at | SAT2 | ENSG00000141504 | 0.037706 | 0.6186121 | 9.078151 | 8.459538 | 17 | p13.1 | spermidine/spermine N1-acetyltransferase family member 2 [Source: HGNC |
| | | | | | | | | | Symbol; Acc: 23160] |
| 204464_s_at | EDNRA | ENSG00000151617 | 0.037706 | 1.4248341 | 7.17677 | 5.751936 | 4 | q31.22 | endothelin receptor type A [Source: HGNC Symbol; Acc: 3179] |
| 205011_at | VWA5A | ENSG00000110002 | 0.037706 | 1.5010688 | 8.178127 | 6.677058 | 11 | q24.2 | von Willebrand factor A domain containing 5A [Source: HGNC Symbol; Acc: 6658] |
| 212765_at | CAMSAP2 | ENSG00000118200 | 0.037714 | 1.0702776 | 7.585165 | 6.514887 | 1 | q32.1 | calmodulin regulated spectrin-associated protein family, member 2 [Source: HGNC |
| | | | | | | | | | Symbol; Acc: 29188] |
| 212624_s_at | CHN1 | ENSG00000128656 | 0.037755 | 1.1993666 | 8.33942 | 7.140053 | 2 | q31.1 | chimerin 1 [Source: HGNC Symbol; Acc: 1943] |
| 209213_at | CBR1 | ENSG00000159228 | 0.037828 | 0.71634 | 9.378176 | 8.661836 | 21 | q22.12 | carbonyl reductase 1 [Source: HGNC Symbol; Acc: 1548] |
| 207738_s_at | NCKAP1 | ENSG00000061676 | 0.037828 | 1.7420168 | 5.909032 | 4.167015 | 2 | q32.1 | NCK-associated protein 1 [Source: HGNC Symbol; Acc: 7666] |
| 206856_at | LILRB5 | ENSG00000105609 | 0.03795 | 1.5873193 | 8.757092 | 7.169773 | 19 | q13.42 | leukocyte immunoglobulin-like receptor, subfamily B (with TM and ITIM domains), |
| | | | | | | | | | member 5 [Source: HGNC: Symbol; Acc: 6609] |
| 200697_at | HK1 | ENSG00000156515 | 0.037989 | 0.4970804 | 10.98017 | 10.48308 | 10 | q22.1 | hexokinase 1 [Source: HGNC Symbol; Acc: 4922] |
| 219134_at | ELTD1 | ENSG00000162618 | 0.03804 | 1.633117 | 7.997273 | 6.364156 | 1 | p31.1 | EGF, latrophilin and seven transmembrane domain containing 1 [Source: HGNC |
| | | | | | | | | | Symbol; Acc: 20822] |
| 222294_s_at | RAB27A | ENSG00000069974 | 0.038154 | 1.5128756 | 7.227373 | 5.714497 | 15 | q21.3 | RAB27A, member RAS oncogene family [Source: HGNC Symbol; Acc: 9766] |
| 201057_s_at | GOLGB1 | ENSG00000173230 | 0.03845 | 0.6396181 | 9.220834 | 8.581216 | 3 | q13.33 | golgin 51 [Source: HGNC Symbol; Acc: 4429] |
| 218017_s_at | HGSNAT | ENSG00000165102 | 0.038567 | 1.101893 | 8.583363 | 7.48147 | 8 | p11.21 | heparan-alpha-glucosamninide N-acetyltransferase [Source: HGNC |
| | | | | | | | | | Symbol; Acc: 26527] |
| 226905_at | FAM101B | ENSG00000183688 | 0.03866 | 1.3556387 | 8.832382 | 7.476743 | 17 | p13.3 | family with sequence similarity 101, member B [Source: HGNC Symbol; Acc: 28705] |
| 226616_s_at | NDUFV3 | ENSG00000160194 | 0.038572 | 0.6620698 | 10.466 | 9.803934 | 21 | q22.3 | NADH dehydrogenase (ubiquinone) flavoprotein 3, 10 kDa [Source: HGNC |
| | | | | | | | | | Symbol; Acc: 7719] |
| 215127_s_at | RBMS1 | ENSG00000153250 | 0.038708 | 0.9857614 | 9.060146 | 8.074385 | 2 | q24.2 | RNA binding motif, single stranded interacting protein 1 [Source: HGNC |
| | | | | | | | | | Symbol; Acc: 9907] |
| 212373_at | FEM1B | ENSG00000169018 | 0.038897 | 0.9618687 | 8.428165 | 7.466297 | 15 | q23 | fern-1 homolog b (C. elegans) [Source: HGNC Symbol; Acc: 3649] |
| 222750_s_at | SRD5A3 | ENSG00000128039 | 0.038897 | 0.7626054 | 8.251817 | 7.489212 | 4 | q12 | steroid 5 alpha-reductase 3 [Source: HGNC Symbol; Acc: 25812] |
| 236006_s_at | AKAP10 | ENSG00000108599 | 0.039007 | 0.6550377 | 7.328287 | 7.173249 | 17 | p11.2 | A kinase (PRKA) anchor protein 10 [Source: HGNC Symbol; Acc: 68] |
| 224733_at | CMTM3 | ENSG00000140931 | 0.039182 | 0.6902697 | 9.982739 | 9.292469 | 16 | q21 | CKLF-like MARVEL transmembrane domain containing 3 [Source: HGNC |
| | | | | | | | | | Symbol; Acc: 19174] |
| 204981_at | SLC22A18 | ENSG00000110628 | 0.039182 | 0.5611839 | 7.58994 | 7.028756 | 11 | p15.4 | solute carrier family 22, member 18 [Source: HGNC Symbol; Acc: 10964] |
| 223077_at | TMOD3 | ENSG00000138594 | 0.03924 | 1.0922066 | 7.964721 | 6.872514 | 15 | q21.2 | tropornodulin 3 (ubiquitous) [Source: HGNC Symbol; Acc: 11873] |
| 209686_at | S100B | ENSG00000160307 | 0.039241 | 0.9012906 | 6.761868 | 5.860578 | 21 | q22.3 | S100 calcium binding protein B [Source: HGNC Symbol; Acc: 10500] |
| 225325_at | MFSD6 | ENSG00000151690 | 0.039241 | 0.8394079 | 8.69193 | 7.852522 | 2 | q32.2 | major facilitator superfamily domain containing 6 [Source: HGNC |
| | | | | | | | | | Symbol; Acc: 24711] |
| 205236_x_at | SOD3 | ENSG00000109610 | 0.03958 | 0.8919006 | 8.451633 | 7.559732 | 4 | p15.2 | superoxide dismutase 3, extracellular [Source: HGNC Symbol; Acc: 11181] |
| 228220_at | FCHO2 | ENSG00000157107 | 0.03958 | 1.5637625 | 8.795856 | 7.232094 | 5 | q13.2 | FCH domain only 2 [Source: HGNC Symbol; Acc: 25180] |
| 205904_at | MICA | ENSG00000204520 | 0.03958 | 0.4272285 | 7.684754 | 7.257525 | 6 | p21.33 | MHC class I polypeptide-related sequence A [Source: HGNC Symbol; Acc: 7090] |
| 226777_at | ADAM12 | ENSG00000148848 | 0.03958 | 1.9432471 | 7.771445 | 5.828198 | 10 | q26.2 | ADAM metallopeptidase domain 12 [Source: HGNC Symbol; Acc: 190] |
| 213618_at | ARAP2 | ENSG00000047365 | 0.03958 | 1.0280821 | 7.620559 | 6.592477 | 4 | p14 | ArfGAP with RhoGAP domain, ankyrin repeat and PH dorna 2 [Source: HGNC |
| | | | | | | | | | Symbol; Acc: 16924] |
| 205624_at | CPA3 | ENSG00000163751 | 0.03958 | 2.1511989 | 6.492962 | 4.341763 | 3 | q24 | carboxypeptidase A3 (mast cell) [Source: HGNC Symbol; Acc: 2298] |
| 215268_at | KIAA0754 | ENSG00000255103 | 0.03958 | 0.8091048 | 6.285141 | 5.476036 | 1 | p34.3 | KIAA0754 [Source: HGNC Symbol; Acc: 29111] |
| 203672_x_at | TPMT | ENSG00000137364 | 0.03958 | 0.5543757 | 9.62339 | 9.069015 | 6 | p22.3 | thiopurine S-methyltransferase [Source: HGNC Symbol; Acc: 12014] |
| 1555229_a_at | C1S | ENSG00000182326 | 0.03958 | 1.5628333 | 10.46271 | 8.899877 | 12 | p13.31 | complement component 1, s subcomponent [Source: HGNC Symbol; Acc: 1247] |
| 203855_at | WDR47 | ENSG00000085433 | 0.03958 | 0.3150022 | 7.128001 | 6.412998 | 1 | p13.3 | WD repeat domain 47 [Source: HGNC Symbol; Acc: 29141] |
| 207072_at | IL18RAP | ENSG00000115607 | 0.03958 | 0.9816906 | 7.548943 | 6.567253 | 2 | q12.1 | interleukin 18 receptor accessory protein [Source: HGNC Symbol; Acc: 5989] |
| 221935_s_at | EOGT | ENSG00000163378 | 0.039608 | 0.8358766 | 7.127104 | 6.291227 | 3 | p14.1 | EGF domain-specific O-linked N-acetylglucosamine (GlcNAc) transferase |
| | | | | | | | | | [Source: HGNC Symbol; Acc: 23526] |
| 203232_s_at | ATXN1 | ENSG00000124788 | 0.039608 | 1.4864727 | 8.293823 | 6.80735 | 6 | p22.3 | ataxin 1 [Source: HGNC Symbol; Acc: 10548] |
| 202096_s_at | TSPO | ENSG00000100300 | 0.03977 | 0.6186136 | 9.561586 | 8.942973 | 22 | q13.2 | translocator protein (18 kDa) [Source: HGNC Symbol; Acc: 1158] |
| 224813_at | WASL | ENSG00000106299 | 0.03977 | 0.8102345 | 8.822272 | 8.012038 | 7 | q31.32 | Wiskott-Aldrich syndrome-like [Source: HGNC Symbol; Acc: 12735] |
| 213455_at | FAM114A1 | ENSG00000197712 | 0.039797 | 0.841538 | 7.947072 | 7.105434 | 4 | p14 | family with sequence similarity 114, member A1 [Source: |
| | | | | | | | | | HGNC Symbol; Acc: 25087] |
| 226873_at | FAM63B | ENSG00000128923 | 0.039878 | 0.7816817 | 7.054056 | 6.272374 | 15 | q21.3 | family with sequence similarity 53, member B [Source: HGNC Symbol; Acc: 26954] |
| 1554503_a_at | OSCAR | ENSG00000170909 | 0.040083 | 0.4815944 | 6.652983 | 6.171388 | 19 | q13.42 | osteoclast associated, immunoglobulin-like receptor [Source: HGNC |
| | | | | | | | | | Symbol; Acc: 29960] |
| 206118_at | STAT4 | ENSG00000138378 | 0.040381 | 1.5428433 | 7.763744 | 6.120901 | 2 | q32.3 | signal transducer and activator of transcription 4 [Source: HGNC |
| | | | | | | | | | Symbol; Acc: 11365] |
| 210970_s_at | IBTK | ENSG00000005700 | 0.040543 | 0.7538865 | 8.260872 | 7.506986 | 6 | q14.1 | inhibitor of Bruton agammaglobulinernia tyrosine kinase [Source: HGNC |
| | | | | | | | | | Symbol; Acc: 17853] |
| 227113_at | ADHFE1 | ENSG00000147576 | 0.040543 | 0.7061362 | 8.382822 | 7.676586 | 8 | q13.1 | alcohol dehydrogenase, iron containing, 1 [Source: HGNC Symbol; Acc: 16354] |
| 202949_s_at | FHL2 | ENSG00000115641 | 0.040594 | 1.4031826 | 7.613598 | 5.210416 | 2 | q12.2 | four and a half LIM domains 2 [Source: HGNC Symbol; Acc: 3703] |
| 225579_at | PQLC3 | ENSG00000162976 | 0.040597 | 1.1136678 | 9.537873 | 8.424205 | 2 | p25.1 | PQ loop repeat containing 3 [Source: HGNC Symbol; Acc: 28503] |
| 235291_s_at | FLJ32255 | NA | 0.040716 | 1.1196959 | 7.867314 | 6.747618 | NA | NA | NA |
| 203939_at | NT5E | ENSG00000135318 | 0.040733 | 0.8061792 | 7.425231 | 6.619052 | 6 | q14.3 | 5′-nucleotidase, ecto (CD73) [Source: HGNC Symbol; Acc: 8021] |
| 214274_s_at | ACAA1 | ENSG00000060971 | 0.040794 | 0.5024191 | 9.455452 | 8.963033 | 3 | p22.2 | acetyl-CoA acyltransferase 1 [Source: HGNC Symbol; Acc: 32] |
| 204046_at | PLCB2 | ENSG00000137841 | 0.040802 | 0.5337836 | 8.533797 | 8.000014 | 15 | q15.1 | phospholipase C, beta 2 [Source: HGNC Symbol; Acc: 9055] |
| 202392_s_at | PISD | ENSG00000241878 | 0.041073 | 0.3563542 | 8.566583 | 8.210219 | 22 | q12.2 | phosphatidylserine decarboxylase [Source: HGNC Symbol; Acc: 8999] |
| 218326_s_at | LGR4 | ENSG00000205213 | 0.041073 | 1.2551214 | 5.02211 | 3.766988 | 11 | p14.1 | leucine-rich repeat containing G protein-coupled receptor 4 [Source: HGNC |
| | | | | | | | | | Symbol; Acc: 13299] |
| 229119_s_at | ZSWIM7 | ENSG00000214941 | 0.041073 | 1.0247863 | 7.91053 | 6.885744 | 17 | p12 | zinc finger, SWIM-type containing 7 [Source: HGNC Symbol; Acc: 26993] |
| 206491_s_at | NAPA | ENSG00000105402 | 0.041415 | 0.4954364 | 9.333764 | 8.838328 | 19 | q13.33 | N-ethylmaleimide-sensitive factor attachment protein, alpha [Source: HGNC |
| | | | | | | | | | Symbol; Acc: 7641] |
| 227450_at | ERP27 | ENSG00000139055 | 0.041415 | 0.9661967 | 6.328914 | 5.362718 | 12 | p12.3 | endoplasmic reticulum protein 27 [Source: HGNC Symbol; Acc: 26495] |
| 200866_s_at | PSAP | ENSG00000197746 | 0.041415 | 1.0853598 | 11.69849 | 10.61313 | 10 | q22.1 | prosaposin [Source: HGNC Symbol; Acc: 9498] |
| 200720_s_at | ACTR1A | ENSG00000138107 | 0.041415 | 0.4967534 | 8.824862 | 8.328108 | 10 | q24.32 | ARP1 actin-related protein 1 homolog A, centractin alpha (yeast) [Source: HGNC |
| | | | | | | | | | Symbol; Acc: 167] |
| 232064_at | FER | ENSG00000151422 | 0.041415 | 0.6560546 | 7.136386 | 6.480331 | 5 | q21.3 | fer (fps/fes related) tyrosine kinase [Source: HGNC Symbol; Acc: 3655] |
| 217922_at | MAN1A2 | ENSG00000198162 | 0.041445 | 0.6973684 | 8.202283 | 7.504915 | 1 | p12 | mannosidase, alpha, class 1A, member 2 [Source: HGNC Symbol; Acc: 822] |
| 201944_at | HEXB | ENSG00000049860 | 0.04157 | 1.0109447 | 11.5932 | 10.58226 | 5 | q13.3 | hexosaminidase B (beta polypeptide) [Source: HGNC Symbol; Acc: 4879] |
| 210986_s_at | TPM1 | ENSG00000140416 | 0.04157 | 1.6503375 | 9.944608 | 8.294271 | 15 | q22.2 | tropomyosin 1 (alpha) [Source: HGNC Symbol; Acc: 12010] |
| 227568_at | HECTD2 | ENSG00000165338 | 0.04157 | 1.1199318 | 6.685061 | 5.565129 | 10 | q23.32 | HECT domain containing E3 ubiquitin protein ligase 2 [Source: HGNC |
| | | | | | | | | | Symbol; Acc: 26736] |
| 200645_at | GABARAP | ENSG00000170296 | 0.04157 | 0.6317473 | 11.38104 | 10.7493 | 17 | p13.1 | GABA(A) receptor-associated protein [Source: HGNC Symbol; Acc: 4067] |
| 219892_at | TM6SF1 | ENSG00000136404 | 0.04157 | 1.5187197 | 7.818959 | 6.300239 | 15 | q25.2 | transmembrane 6 superfamily member 1 [Source: HGNC Symbol; Acc: 11860] |
| 222431_at | SPIN1 | ENSG00000106723 | 0.04157 | 0.7028249 | 9.18628 | 8.483455 | 9 | q22.1 | spindlin 1 [Source: HGNC Symbol; Acc: 11243] |
| 203665_at | HMOX1 | ENSG00000100292 | 0.04157 | 1.0199085 | 9.506814 | 8.486905 | 22 | q12.3 | heme oxygenate (decycling) 1 [Source: HGNC Symbol; Acc: 5013] |
| 202506_at | SSFA2 | ENSG00000138434 | 0.04157 | 0.9475539 | 8.018471 | 7.070918 | 2 | q31.3 | sperm specific antigen 2 [Source: HGNC Symbol; Acc: 11319] |
| 221899_at | N4BP2L2 | ENSG00000244754 | 0.04157 | 0.8653353 | 9.046774 | 8.181439 | 13 | q13.1 | NEDD4 binding protein 2-like 2 [Source: HGNC Symbol; Acc: 26916] |
| 203668_at | MAN2C1 | ENSG00000140400 | 0.04169 | 0.6339166 | 8.66367 | 8.029753 | 15 | q24.2 | mannosidase, alpha, class 2C, member 1 [Source: HGNC Symbol; Acc: 6827] |
| 206295_at | IL18 | ENSG00000150782 | 0.041917 | 1.2921744 | 9.298736 | 8.006561 | 11 | q23.1 | interleukin 18 (interferon-gamma-inducing factor) [Source: HGNC |
| | | | | | | | | | Symbol; Acc: 5986] |
| 228152_s_at | DDX60L | ENSG00000181381 | 0.041917 | 1.4213436 | 7.853792 | 6.432448 | 4 | q32.3 | DEAD (Asp-Glu-Ala-Asp) box polypeptide 60-like [Source: |
| | | | | | | | | | HGNC Symbol; Acc: 26429] |
| 228341_at | NUDT16 | ENSG00000198585 | 0.041917 | 0.8132856 | 6.707602 | 5.894316 | 3 | q22.1 | nudix (nucleoside diphosphate linked moiety X)-type motif 16 [Source: HGNC |
| | | | | | | | | | Symbol; Acc: 26442] |
| 218164_at | SPATA20 | ENSG00000006282 | 0.041917 | 0.5879296 | 9.023334 | 8.435404 | 17 | q21.33 | spermatogenesis associated 20 [Source: HGNC Symbol; Acc: 26125] |
| 227070_at | GLT8D2 | ENSG00000120820 | 0.041917 | 1.7573511 | 5.840318 | 4.082966 | 12 | q23.3 | glycosyltransferase 8 domain containing 2 [Source: HGNC Symbol; Acc: 24890] |
| 213083_at | SLC35D2 | ENSG00000130958 | 0.041917 | 0.8293654 | 7.315854 | 6.486489 | 9 | q22.32 | solute carrier family 35 (UDP-GlcNAc/UDP-glucose transporter), member D2 |
| | | | | | | | | | [Source: HGNC Symbol; Acc: 20799] |
| 224772_at | NAV1 | ENSG00000134369 | 0.041917 | 1.0514789 | 7.602146 | 6.550667 | 1 | q32.1 | neuron navigator 1 [Source: HGNC Symbol; Acc: 15989] |
| 229287_at | PCNX | ENSG00000100731 | 0.041917 | 0.9439305 | 7.156202 | 6.622271 | 14 | q24.2 | pecanex homolog (Drosophila) [Source: HGNC Symbol; Acc: 19740] |
| 201200_at | CREG1 | ENSG00000143162 | 0.041917 | 1.0581554 | 11.10069 | 10.04253 | 1 | q24.2 | cellular repressor of E1A-stimulated genes 1 [Source: HGNC Symbol; Acc: 2351] |
| 229450_at | IFIT3 | ENSG00000119917 | 0.041917 | 1.6631975 | 9.084235 | 7.421037 | 10 | q23.31 | interferon-induced protein with tetratricopeptide repeats 3 [Source: HGNC |
| | | | | | | | | | Symbol; Acc: 5411] |
| 228249_at | C11orf74 | ENSG00000166352 | 0.041917 | 0.8487982 | 5.174339 | 4.325541 | 11 | p12 | chromosome 11 open reading frame 74 [Source: HGNC Symbol; Acc: 25142] |
| 38487_at | STAB1 | ENSG00000010327 | 0.042059 | 1.0905804 | 9.31099 | 8.22041 | 3 | p21.1 | stabilin 1 [Source: HGNC Symbol; Acc: 18628] |
| 202974_at | MPP1 | ENSG00000130830 | 0.042059 | 1.0629761 | 9.61456 | 8.551584 | X | q28 | membrane protein, palmitoylated 1, 55 kDa [Source: HGNC Symbol; Acc: 7219] |
| 226510_at | HEATR5A | ENSG00000129493 | 0.042059 | 0.7510398 | 7.259761 | 6.508721 | 14 | q12 | HEAT repeat containing 5A [Source: HGNC Symbol; Acc: 20276] |
| 228282_at | MFSD8 | ENSG00000164073 | 0.042059 | 0.8192732 | 7.582661 | 6.763388 | 4 | q28.2 | major facilitator superfamily domain containing 8 [Source: HGNC |
| | | | | | | | | | Symbol; Acc: 284,86] |
| 203675_at | NUCB2 | ENSG00000070081 | 0.042059 | 0.9057747 | 9.751698 | 8.845924 | 11 | p15.1 | nucleobindin 2 [Source: HGNC Symbol; Acc: 8044] |
| 228384_s_at | PYROXD2 | ENSG00000119943 | 0.042059 | 0.3040111 | 6.385891 | 6.08188 | 10 | q24.2 | pyridine nucleotide-disulphide oxidoreductase domain 2 [Source: HGNC |
| | | | | | | | | | Symbol; Acc: 23517] |
| 229367_s_at | GIMAP6 | ENSG00000133561 | 0.042059 | 1.4090136 | 8.888668 | 7.479654 | 7 | q36.1 | GTPase, IMAP family member 6 [Source: HGNC Symbol; Acc: 21918] |
| 218309_at | CAMK2N1 | ENSG00000162545 | 0.042059 | 1.2605333 | 7.868837 | 6.608303 | 1 | p36.12 | calcium/calmodulin-dependent protein kinase H inhibitor 1 [Source: HGNC |
| | | | | | | | | | Symbol; Acc: 24190] |
| 34726_at | CACNB3 | ENSG00000167535 | 0.042059 | 0.4136308 | 6.934617 | 6.520986 | 12 | q13.12 | calcium channel, voltage-dependent, beta 3 subunit [Source: HGNC |
| | | | | | | | | | Symbol; Acc: 1403] |
| 212670_at | ELN | ENSG00000049540 | 0.042059 | 1.2311529 | 8.8137 | 7.582547 | 7 | q11.23 | elastin [Source: HGNC Symbol; Acc: 3327] |
| 229800_at | DCLK1 | ENSG00000133083 | 0.042059 | 1.4985924 | 6.412071 | 4.913479 | 13 | q13.3 | doublecortin-like kinase 1 [Source: HGNC Symbol; Acc: 2700] |
| 201975_at | CLIP1 | ENSG00000130779 | 0.042246 | 1.0285112 | 8.482483 | 7.453972 | 12 | q24.31 | CAP-GLY domain containing linker protein 1 [Source: HGNC Symbol; Acc: 10461] |
| 224413_s_at | TM2D2 | ENSG00000169490 | 0.042248 | 0.6130057 | 9.024042 | 8.411037 | 8 | p11.22 | TM2 domain containing 2 [Source: HGNC Symbol; Acc: 24127] |
| 204011_at | SPRY2 | ENSG00000136158 | 0.042332 | 1.3188526 | 7.198128 | 5.879276 | 13 | q31.1 | sprouty homolog 2 (Drosophila) [Source: HGNC Symbol; Acc: 11270] |
| 235033_at | NPEPL1 | ENSG00000215440 | 0.042332 | 0.7632057 | 5.890585 | 5.12738 | 20 | q13.32 | aminopeptidase-like 1 [Source: HGNC Symbol; Acc: 16244] |
| 228348_at | LINS | ENSG00000140471 | 0.042351 | 0.8898302 | 8.824668 | 7.934838 | 15 | q26.3 | lines homolog (Drosophila] [Source: HGNC. Symbol; Acc: 30922] |
| 227628_at | GPX8 | ENSG00000164294 | 0.042351 | 1.180448 | 6.970795 | 5.790347 | 5 | q11.2 | glutathione peroxidase 8 (putative) (Source: HGNC Symbol; Acc: 33100] |
| 1557112_a_at | VPS53 | ENSG00000141252 | 0.042351 | 0.5180325 | 7.975808 | 7.457775 | 17 | p13.3 | vacuolar protein sorting 53 homolog (S. cerevisiae) [Source: HGNC |
| | | | | | | | | | Symbol; Acc: 25608] |
| 228071_at | GIMAP7 | ENSG00000179144 | 0.042351 | 1.647364 | 8.992109 | 7.344745 | 7 | q36.1 | GTPase, IMAP family member 7 [Source: HGNC Symbol; Acc: 22404] |
| 209264_s_at | TSPAN4 | ENSG00000214063 | 0.042351 | 0.6817548 | 9.0067 | 8.324945 | 11 | p15.5 | tetraspanin 4 [Source: HGNC Symbol; Acc: 11859] |
| 208892_s_at | DUSP6 | ENSG00000139318 | 0.042351 | 1.2289738 | 7.99204 | 6.763066 | 12 | q21.33 | dual specificity phosphatase 6 [Source: HGNC Symbol; Acc: 3072] |
| 201315_x_at | IFITM2 | ENSG00000185201 | 0.042351 | 0.7393922 | 11.67358 | 10.93419 | 11 | p15.5 | interferon induced transmembrane protein 2 [Source: HGNC Symbol; Acc: 5413] |
| 222802_at | EDN1 | ENSG00000078401 | 0.042351 | 1.4605418 | 6.322746 | 4.862204 | 6 | p24.1 | endothelin 1 [Source: HGNC Symbol; Acc: 3176] |
| 1553395_a_at | CD200R1 | ENSG00000163606 | 0.042351 | 1.2569103 | 6.464779 | 5.207868 | 3 | q13.2 | CD200 receptor 1 [Source: HGNC,Symbol; Acc: 24235] |
| 226399_at | DNAJB14 | ENSG00000164031 | 0.042351 | 0.8499477 | 8.162884 | 7.312937 | 4 | q23 | DnaJ (Hsp40) homolog, subfamily B, member 14 [Source: HGNC Symbol; |
| | | | | | | | | | Acc: 25881] |
| 226490_at | NHSL1 | ENSG00000135540 | 0.042351 | 1.1946659 | 7.117631 | 5.922965 | 6 | q24.1 | NHS-like 1 [Source: HGNC Symbol; Acc: 21021] |
| 222513_s_at | SORBS1 | ENSG00000095637 | 0.042351 | 1.325461 | 8.112293 | 6.786832 | 10 | q24.1 | sorbin and SH3 domain containing 1 [Source: HGNC Symbol; Acc: 14565] |
| 218705_s_at | SNX24 | ENSG00000064652 | 0.042351 | 0.9094316 | 7.531422 | 6.62199 | 5 | q23.2 | sorting nexin 24 [Source: HGNC Symbol; Acc: 21533] |
| 226837_at | SPRED1 | ENSG00000166068 | 0.042351 | 1.2128712 | 7.561562 | 6.34869 | 15 | q14 | sprouty-related, EVH1 domain containing 1 [Source: HGNC Symbol; Acc: 20249] |
| 225338_at | ZYG11B | ENSG00000162378 | 0.042351 | 0.6244781 | 7.31379 | 6.689312 | 7 | p32.3 | zyg-11 family member B, cell cycle regulator [Source: HGNC Symbol; Acc: 25820] |
| 204955_at | SRPX | ENSG00000101955 | 0.042351 | 1.7691767 | 7.330012 | 5.560835 | X | p11.4 | sushi-repeat containing protein, X-linked [Source: HGNC Symbol; Acc: 11309] |
| 210840_s_at | IQGAP1 | ENSG00000140575 | 0.042351 | 0.8156678 | 10.60381 | 9.788142 | 15 | q26.1 | IQ motif containing GTPase activating protein 1 [Source: HGNC Symbol; Acc: 6110] |
| 201494_at | PRCP | ENSG00000137509 | 0.042351 | 0.8806117 | 10.84225 | 9.961639 | 11 | q14.1 | prolylcarboxypeptidase (angiotensinase C) [Source: HGNC Symbol; Acc: 9344] |
| 204415_at | IFI6 | ENSG00000126709 | 0.042351 | 1.1130836 | 9.366478 | 8.253394 | 1 | p36.11 | interferon, alpha-inducible protein 6 [Source: HGNC Symbol; Acc: 4054] |
| 209290_s_at | NFIB | ENSG00000147862 | 0.042351 | 1.6557745 | 8.409196 | 6.753422 | 9 | p22.3 | nuclear factor 1/13 [Source: HGNC Symbol; Acc: 7785] |
| 221474_at | MYL12B | ENSG00000118680 | 0.04242 | 0.5861311 | 10.97279 | 10.38666 | 18 | p11.31 | myosin, light chain 12B, regulatory [Source: HGNC Symbol; Acc: 29827] |
| 235747_at | SLC25A16 | ENSG00000122912 | 0.04242 | 0.5460494 | 7.042745 | 6.496696 | 10 | q21.3 | solute carrier family 25 (mitochondrial carrier; Graves disease autoantigen), |
| | | | | | | | | | member 16 [Source: HGNC Symbol; Acc: 10986] |
| 201924_at | AFF1 | ENSG00000172493 | 0.04242 | 0.9520638 | 10.24162 | 9.289557 | 4 | q21.3 | AF4/FMR2 family, member 1 [Source: HGNC Symbol; Acc: 7135] |
| 218045_x_at | PTMS | ENSG00000159335 | 0.042596 | 0.6071624 | 9.924388 | 9.317226 | 12 | p13.31 | parathymosin [Source: HGNC Symbol; Acc: 9629] |
| 213943_at | TWIST1 | ENSG00000122691 | 0.042596 | 1.1687342 | 7.271938 | 6.103204 | 7 | p21.1 | twist basic helix-loop-helix transcription factor 1 [Source: HGNC |
| | | | | | | | | | Symbol; Acc: 12428] |
| 226752_at | FAM174A | ENSG00000174132 | 0.042596 | 0.9287163 | 5.776449 | 4.847732 | 5 | q21.1 | family with sequence similarity 174, member A [Source: HGNC |
| | | | | | | | | | Symbol; Acc: 24943] |
| 201876_at | PON2 | ENSG00000105854 | 0.04272 | 1.1875711 | 9.054738 | 7.867167 | 7 | q21.3 | paraoxonase 2 [Source: HGNC Symbol; Acc: 9205] |
| 1554240_a_at | ITGAL | ENSG00000005844 | 0.042882 | 1.3025054 | 9.317163 | 8.014657 | 16 | p11.2 | integrin, alpha L (antigen CD11A (p180), lymphocyte function-associated antigen |
| | | | | | | | | | 1; alpha polypeptide) [Source: HGNC Symbol; Acc: 6148] |
| 203989_x_at | F2R | ENSG00000181104 | 0.042891 | 1.2465199 | 7.440039 | 6.193519 | 5 | q13.3 | coagulation factor II (thrombin) receptor [Source: HGNC Symbol; Acc: 3537] |
| 226425_at | CLIP4 | ENSG00000115295 | 0.042891 | 1.4998601 | 7.783328 | 6.283468 | 2 | p23.2 | CAP-GLY domain containing linker protein family, member 4 [Source: HGNC |
| | | | | | | | | | Symbol; Acc: 26108] |
| 200762_at | DPYSL2 | ENSG00000092964 | 0.042891 | 1.1591718 | 9.328124 | 8.168952 | 8 | p21.2 | dihydropyrimidinase-like 2 [Source: HGNC Symbol; Acc: 3014] |
| 223681_s_at | INADL | ENSG00000132849 | 0.042891 | 1.2509988 | 6.181877 | 4.930378 | 1 | p31.3 | InaD-like (Drosophila) [Source: HGNC Symbol; Acc: 28881] |
| 211986_at | AHNAK | ENSG00000124942 | 0.042891 | 1.3341466 | 11.47856 | 10.14441 | 11 | q12.3 | AHNAK nucleoprotein [Source: HGNC Symbol; Acc: 347] |
| 204112_s_at | HNMT | ENSG00000150540 | 0.042891 | 1.303475 | 8.681933 | 7.378458 | 2 | q22.1 | histamine N-methyltransferase [Source: HGNC Symbol; Acc: 5028] |
| 235360_at | PLEKHM3 | ENSG00000178385 | 0.042891 | 0.4363264 | 7.481078 | 7.044751 | 2 | q33.3 | pleckstrin homology domain containinng, family M, member 3 [Source: HGNC |
| | | | | | | | | | Symbol; Acc: 34006] |
| 209276_s_at | GLRX | ENSG00000173221 | 0.04294 | 1.1533528 | 10.46513 | 9.31178 | 5 | q15 | glutaredoxin (thioltransferase) [Source: HGNC Symbol; Acc: 4330] |
| 226113_at | ZNF436 | ENSG00000125945 | 0.042951 | 0.9676351 | 7.934453 | 6.966818 | 1 | p36.12 | zinc finger protein 436 [Source: HGNC Symbol; Acc: 20814] |
| 201694_s_at | EGR1 | ENSG00000120738 | 0.043033 | 2.3562082 | 9.680723 | 7.324515 | 5 | q31.2 | early growth response 1 [Source: HGNC: Symbol; Acc: 3238] |
| 204417_at | GALC | ENSG00000054983 | 0.043091 | 1.1819717 | 9.186049 | 8.004078 | 14 | q31.3 | galactosylceramidase [Source: HGNC Symbol; Acc: 4115] |
| 211178_s_at | PSTPIP1 | ENSG00000140368 | 0.043103 | 0.5580401 | 8.764375 | 8.206335 | 15 | q24.3 | proline-serine-threonine phosphatase interacting protein 1 [Source: HGNC |
| | | | | | | | | | Symbol; Acc: 9580] |
| 203088_at | FBLN5 | ENSG00000140092 | 0.043103 | 1.5746104 | 8.128332 | 6.553721 | 14 | q32.12 | fibulin 5 [Source: HGNC Symbol; Acc: 3602] |
| 218450_at | HEBP1 | ENSG00000013583 | 0.043103 | 0.7563211 | 8.459513 | 7.703192 | 12 | p13.1 | heme binding protein 1 [Source: HGNC Symbol; Acc: 17176] |
| 210113_s_at | NLRP1 | ENSG00000091592 | 0.043123 | 0.6544652 | 7.398937 | 6.744472 | 17 | p13.2 | NLR family, pyrin domain containing 1 [Source: HGNC Symbol; Acc: 14374] |
| 225673_at | MYADM | ENSG00000179820 | 0.043128 | 1.1859735 | 9.60657 | 8.274684 | 19 | q13.42 | myeloid-associated differentiation marker [Source: HGNC Symbol; Acc: 7544] |
| 31874_at | GAS2L1 | ENSG00000185340 | 0.043128 | 0.9591258 | 7.063628 | 6.104502 | 22 | q12.2 | growth arrest-specific 2 like 1 [Source: HGNC Symbol; Acc: 16955] |
| 222597_at | SNAP29 | ENSG00000099940 | 0.043128 | 0.523138 | 8.682269 | 8.159131 | 22 | q11.21 | synantosomal-associated protein, 29 kDa [Source: HGNC Symbol; Acc: 11133] |
| 226279_at | PRSS23 | ENSG00000150687 | 0.043153 | 1.3604367 | 7.675135 | 6.314698 | 11 | q14.2 | protease, serine, 23 [Source: HGNC. Symbol; Acc: 14370] |
| 235570_at | RSMS3 | ENSG00000144642 | 0.043153 | 1.7823279 | 7.335633 | 5.553305 | 3 | p24.1 | RNA binding motif, single stranded interacting protein 3 [Source: HGNC |
| | | | | | | | | | Symbol; Acc: 134271 |
| 214830_at | SLC38A6 | ENSG00000139974 | 0.04325 | 1.2769308 | 7.341343 | 6.064412 | 14 | q23.1 | solute carrier family 38, member 6 [Source: HGNC Symbol; Acc: 19863] |
| 201366_at | ANXA7 | ENSG00000138279 | 0.04326 | 0.7630117 | 8.850377 | 8.087365 | 10 | q22.2 | annexin A7 [Source: HGNC Symbol; Acc: 545] |
| 203179_at | GALT | ENSG00000213930 | 0.043316 | 0.4101715 | 8.833284 | 8.423113 | 9 | p13.3 | galactose-1-phosphate uridyltransferase Source: HGNC Symbol; Acc: 4135] |
| 225919_s_at | C9orf72 | ENSG00000147894 | 0.043475 | 0.8552519 | 8.18141 | 7.326158 | 9 | p21.2 | chromosome 9 open reading frame 72 Source: HGNC Symbol; Acc: 28337] |
| 36711_at | MAFF | ENSG00000185022 | 0.043475 | 1.042386 | 5.605979 | 4.563593 | 22 | q13.1 | v-maf avian musculoaponeurotic fibrosarcoma oncogene homolog F |
| | | | | | | | | | [Source: HGNC Symbol; Acc: 6780] |
| 205174_s_at | QPCT | ENSG00000115828 | 0.043531 | 1.2617245 | 8.418599 | 7.156874 | 2 | p22.2 | glutaminyl-peptide cyclotransferase Source: HGNC Symbol; Acc: 97531 |
| 225032_at | FNDC3B | ENSG00000075420 | 0.043657 | 1.1481117 | 9.690617 | 8.542506 | 3 | q26.31 | fibronectin type III domain containing 3B [Source: HGNC Symbol; Acc: 46701 |
| 225283_at | ARRDC4 | ENSG00000140450 | 0.043675 | 1.2632774 | 6.919418 | 5.656141 | 15 | q26.6 | arrestin domain containing 4 Source: HGNC Symbol; Acc: 28087] |
| 212820_at | DMXL2 | ENSG00000104093 | 0.043693 | 1.7237553 | 9.098754 | 7.374999 | 15 | q21.2 | Dmx-like 2 [Source: HGNC Symbol; Acc: 2938] |
| 202411_at | IFI27 | ENSG00000165949 | 0.043693 | 1.4199045 | 10.65301 | 9.233109 | 14 | q32.12 | interferon, alpha-inducible protein 27 [Source: HGNC Symbol; Acc: 5397] |
| 201444_s_at | ATP6AP2 | ENSG00000182220 | 0.043693 | 0.8481282 | 9.927791 | 9.079663 | X | p11.4 | ATPase, H transporting, lysosomal accessory protein 2 [Source: HGNC |
| | | | | | | | | | Symbol; Acc: 18305] |
| 212209_at | MED13L | ENSG00000123066 | 0.043693 | 0.718689 | 7.683203 | 6.964514 | 12 | q24.21 | mediator complex subunit 13-like[Source: HGNC Symbol; Acc: 22962] |
| 228082_at | CLMP | ENSG00000166250 | 0.043693 | 0.811086 | 8.019361 | 7.208275 | 11 | q24.1 | CXADR-like membrane protein Source: HGNC Symbol; Acc: 24039] |
| 201579_at | FAT1 | ENSG00000083857 | 0.043693 | 1.1996966 | 6.984438 | 5.784741 | 4 | q35.2 | FAT atypical cadherin 1 [Source: HGNC Symbol; Acc: 3595] |
| 201050_at | PLD3 | ENSG00000105223 | 0.043739 | 0.872713 | 11.22151 | 10.34879 | 19 | q13.2 | phospholipase D family, member 3 [Source: HGNC Symbol; Acc: 17158] |
| 205726_at | DIAPH2 | ENSG00000147202 | 0.043865 | 0.8243238 | 7.524442 | 6.700118 | X | q21.33 | diaphanous-related formin 2 [Source: HGNC Symbol; Acc: 2877] |
| 212717_at | PLEKHM1 | ENSG00000225190 | 0.043869 | 0.3500827 | 8.468551 | 8.118469 | 17 | q21.31 | pleckstrin homology domain containing, family M (with RUN domain) member 1 |
| | | | | | | | | | [Source: HGNC Symbol; Acc: 29017] |
| 202827_s_at | MMP14 | ENSG00000157227 | 0.04395 | 1.0934303 | 9.702738 | 8.609308 | 14 | q11.2 | matrix metallopepticiase 14 (membrane-inserted) [Source: HGNC |
| | | | | | | | | | Symbol; Acc: 7160] |
| 201594_s_at | PPP4R1 | ENSG00000154845 | 0.04395 | 0.8180394 | 9.274119 | 8.456079 | 18 | p11.22 | protein phosphatase 4, regulatory subunit 1 [Source: HGNC Symbol; Acc: 9320] |
| 203460_s_at | PSEN1 | ENSG00000080815 | 0.04395 | 0.8139024 | 8.845797 | 8.031895 | 14 | q24.2 | presenilin 1[Source: HGNC Symbol; Acc: 95081 |
| 221840_at | PTPRE | ENSG00000132334 | 0.044017 | 1.2948506 | 8.885638 | 7.590787 | 10 | q26.2 | protein tyrosine phosphatase, receptor type, E [Source: HGNC Symbol; Acc: 9669] |
| 203410_at | AP3M2 | ENSG00000070718 | 0.044043 | 0.7683391 | 7.036583 | 6.268244 | 8 | p11.21 | adaptor-related protein complex 3, mu 2 subunit [Source: HGNC Symbol; Acc: 570] |
| 226582_at | LOC400043 | NA | 0.044043 | 0.8694792 | 6.734431 | 5.864952 | NA | NA | NA |
| 209600_s_at | ACOX1 | ENSG00000161533 | 0.044043 | 0.6483325 | 8.512058 | 7.863726 | 17 | q25.1 | acyl-CoA oxidase 1, palmitoyl [Source: HGNC Symbol; Acc: 119] |
| 221814_at | GPR124 | ENSG00000020181 | 0.044043 | 1.0958806 | 8.488648 | 7.392767 | 8 | p11.23 | G protein-coupled receptor 124 [Source: HGNC Symbol; Acc: 17849] |
| 230836_at | ST8SIA4 | ENSG00000113532 | 0.044043 | 1.0174978 | 7.57919 | 6.561693 | 5 | q21.1 | ST8 alpha-N-acetyl-neuraminide alpha-2,8-sialyltransferase 4 [Source: HGNC |
| | | | | | | | | | Symbol; Acc: 10871] |
| 204270_at | SKI | ENSG00000157933 | 0.044043 | 0.8960351 | 9.220481 | 8.324446 | 1 | p36.33 | v-ski avian sarcoma viral oncogene homolog [Source: HGNC Symbol; Acc: 10896] |
| 200872_at | S100A10 | ENSG00000197747 | 0.044043 | 0.8622314 | 12.14558 | 11.28335 | 1 | q21.3 | S100 calcium binding protein A10 [Source: HGNC Symbol; Acc: 10487] |
| 219761_at | CLEC1A | ENSG00000150048 | 0.044043 | 1.2912893 | 6.704346 | 5.413057 | 12 | p13.2 | C-type lectin domain family 1, member A [Source: HGNC Symbol; Acc: 24355] |
| 212708_at | MSL1 | ENSG00000188895 | 0.044043 | 0.8560698 | 9.529726 | 8.673656 | 17 | q21.1 | male-specific lethal 1 homolog (Drosophila) [Source: HGNC Symbol; Acc: 27905] |
| 204326_x_at | MT1X | ENSG00000187193 | 0.04407 | 1.0017293 | 10.86485 | 9.863125 | 16 | q13 | metallothionein 1X [Source: HGNC Symbol; Acc: 7405] |
| 241392_at | TMEM39A | ENSG00000176142 | 0.04407 | 0.341471 | 6.928602 | 6.587131 | 3 | q13.33 | transmembrane protein 39A [Source: HGNC Symbol; Acc: D600] |
| 205771_s_at | AKAP7 | ENSG00000118507 | 0.04407 | 0.7319958 | 6.526725 | 5.794729 | 6 | q23.2 | A kinase (PRKA) anchor protein 7 [Source: HGNC Symbol; Acc: 377] |
| 206227_at | CILP | ENSG00000138615 | 0.04407 | 2.2575028 | 8.735998 | 6.478495 | 15 | q22.31 | cartilage intermediate layer protein, nucleotide pyrophosphohydrolase |
| | | | | | | | | | [Source: HGNC Symbol; Acc: 1980] |
| 204158_s_at | TORG1 | ENSG00000110719 | 0.04407 | 0.5930798 | 9.597157 | 9.004077 | 11 | q13.2 | T-cell, immune regulator 1, ATPase, H transporting, lysosomal V0 subunit A3 |
| | | | | | | | | | [Source: HGNC Symbol; Acc: 11647] |
| 212948_at | CAMTA2 | ENSG00000108509 | 0.04407 | 0.5383882 | 9.486336 | 8.947948 | 17 | p13.2 | calmodulin binding transcription activator 2 [Source: HGNC Symbol; Acc: 18807] |
| 218241_at | GOLGA5 | ENSG00000066455 | 0.044078 | 0.5034783 | 7.912464 | 7.408985 | 14 | q32.12 | golgin A5 [Source: HGNC Symbol; Acc: 4428] |
| 203042_at | LAMP2 | ENSG00000005893 | 0.044078 | 1.3866138 | 9.189891 | 7.803278 | X | q24 | lysosomal-associated membrane protein 2 [Source: HGNC Symbol; Acc: 6501] |
| 223264_at | MESDC1 | ENSG00000140406 | 0.044215 | 0.6585783 | 8.668695 | 8.010116 | 15 | q25.1 | mesoderm development candidate 1 [Source: HGNC Symbol; Acc: 13519] |
| 243141_at | SGMS2 | ENSG00000164023 | 0.044313 | 1.0856346 | 5.816519 | 4.730885 | 4 | q25 | sphingomyelin synthase 2 [Source: HGNC Symbol; Acc: 28395] |
| 212513_s_at | USP33 | ENSG00000077254 | 0.044313 | 0.8692058 | 8.910601 | 8.041395 | 1 | p31.1 | ubiquitin specific peptidase 33 [Source: HGNC Symbol; Acc: 20059] |
| 44111_at | VPS33B | ENSG00000184056 | 0.044313 | 0.7185294 | 7.769682 | 7.051153 | 15 | q26.1 | vacuolar protein sorting 33 homolog B (yeast) [Source: HGNC Symbol; Acc: 12712] |
| 203044_at | CHSY1 | ENSG00000131873 | 0.044778 | 0.8603129 | 9.374891 | 8.514578 | 15 | q26.3 | chondroitin sulfate synthase 1 [Source: HGNC Symbol; Acc: 17198] |
| 201133_s_at | PJA2 | ENSG00000198961 | 0.045063 | 0.9288514 | 8.789327 | 7.860476 | 5 | q21.3 | praja ring finger 2, E3 ubiquitin protein ligase [Source: HGNC Symbol; Acc: 17481] |
| 238478_at | BNC2 | ENSG00000173068 | 0.045109 | 1.5819289 | 6.500666 | 4.918737 | 9 | p22.2 | basonuclin 2 [Source: HGNC Symbol; Acc: 30988] |
| 207121_s_at | MAPK6 | ENSG00000069956 | 0.045217 | 0.9719614 | 9.250325 | 8.278364 | 15 | q21.2 | mitogen-activated protein kinase 6 [Source: HGNC Symbol; Acc: 6879] |
| 226757_at | IFIT2 | ENSG00000119922 | 0.045217 | 1.2581633 | 7.398381 | 6.140218 | 10 | q23.31 | interferon-induced protein with tetratricopeptide repeats 2 [Source: HGNC |
| | | | | | | | | | Symbol; Acc: 5409] |
| 224929_at | TMEM173 | ENSG00000184584 | 0.045217 | 0.9769018 | 8.795662 | 7.81876 | 5 | q31.2 | transmembrane protein 173 [Source: HGNC Symbol; Acc: 27962] |
| 219013_at | GALNT11 | ENSG00000178234 | 0.045217 | 0.6247652 | 8.111616 | 7.486851 | 7 | q22.31 | UDP-N-acetyl-alpha-D-galactosamine: polypeptide N- |
| | | | | | | | | | acetylgalactosaminyltransferase 11 (GalNAc-T11) [Source: HGNC |
| | | | | | | | | | Symbol; Acc: 19875] |
| 203732_at | TRIP4 | ENSG00000103671 | 0.045217 | 0.5349917 | 8.282764 | 7.747772 | 15 | q22.31 | thyroid hormone receptor interactor 4 [Source: HGNC Symbol; Acc: 12310] |
| 206991_s_at | CCR5 | ENSG00000160791 | 0.045321 | 1.4988998 | 8.807196 | 7.308296 | 3 | p21.31 | chemokine (C-C motif) receptor 5 (gene/pseudogene) [Source: HGNC |
| | | | | | | | | | Symbol; Acc: 1606] |
| 212192_at | KCTD12 | ENSG00000178695 | 0.045321 | 1.241112 | 11.04262 | 9.801504 | 13 | q22.3 | potassium channel tetramerization domain containing 12 [Source: HGNC |
| | | | | | | | | | Symbol; Acc: 14678] |
| 212071_s_at | SPTBN1 | ENSG00000115306 | 0.045389 | 1.1482987 | 10.50839 | 9.360088 | 2 | p16.2 | spectrin, beta, non-erythrocytic 1 [Source: HGNC Symbol; Acc: 11275] |
| 204194_at | BACH1 | ENSG00000156273 | 0.045389 | 0.6844954 | 7.33134 | 6.646344 | 21 | q21.3 | BTB and CNC homology 1, basic leucine zipper transcription factor 1 |
| | | | | | | | | | [Source: HGNC Symbol; Acc: 935] |
| 213469_at | PGAP1 | ENSG00000197121 | 0.045389 | 0.8013379 | 4.677932 | 3.876544 | 2 | q33.1 | post-GPI attachment to proteins 1 [Source: HGNC Symbol; Acc: 25712] |
| 207173_x_at | CDH11 | ENSG00000140937 | 0.045389 | 1.855492 | 9.112093 | 7.256601 | 16 | q21 | cadherin 11, type 2, OB-cadherin (osteoblast) [Source: HGNC Symbol; Acc: 1750] |
| 202020_s_at | LANCL1 | ENSG00000115365 | 0.045389 | 0.7225894 | 9.184946 | 8.462357 | 2 | q34 | LanC lantibiotic synthetase component C- like 1 (bacterial) [Source: HGNC |
| | | | | | | | | | Symbol; Acc: 6508] |
| 201384_s_at | NBR1 | ENSG00000188554 | 0.045622 | 0.6971938 | 9.452834 | 8.75564 | 17 | q21.31 | neighbor of BRCA1 gene 1 [Source: HGNC Symbol; Acc: 6746] |
| 213004_at | ANGPTL2 | ENSG00000136859 | 0.04565 | 1.4882714 | 9.435206 | 7.946935 | 9 | q33.3 | angiopoietin-like 2 [Source: HGNC Symbol; Acc: 490] |
| 203310_at | STXBP3 | ENSG00000116266 | 0.045775 | 0.7707987 | 7.721639 | 6.950841 | 1 | p13.3 | syntaxin binding protein 3 [Source: HGNC Symbol; Acc: 11446] |
| 212239_at | PIK3R1 | ENSG00000145675 | 0.045775 | 0.7377508 | 9.025386 | 8.287636 | 5 | q13.1 | phosphoinositide-3-kinase, regulatory subunit 1 (alpha) [Source: HGNC |
| | | | | | | | | | Symbol; Acc: 8979] |
| 225864_at | FAM84B | ENSG00000168672 | 0.045775 | 1.6779147 | 6.194231 | 4.516317 | 8 | q24.21 | family with sequence similarity 84, member B [Source: HGNC Symbol; Acc: 24166] |
| 218679_s_at | VPS28 | ENSG00000160948 | 0.04581 | 0.3909079 | 9.69126 | 9.300352 | 8 | q24.3 | vacuolar protein sorting 28 homolog (S. cerevisiae) [Source: HGNC |
| | | | | | | | | | Symbol; Acc: 18178] |
| 211964_at | COL4A2 | ENSG00000134871 | 0.045857 | 1.2328119 | 10.70556 | 9.472749 | 13 | q34 | collagen, type IV, alpha 2 [Source: HGNC Symbol; Acc: 2203] |
| 212501_at | CEBPB | ENSG00000172216 | 0.045857 | 0.7382385 | 10.55473 | 9.816494 | 20 | q13.13 | CCAAT/enhancer binding protein (C/EBP), beta [Source: HGNC Symbol; Acc: 1834] |
| 215596_s_at | LTN1 | ENSG00000198862 | 0.045857 | 0.6006951 | 9.123146 | 8.522451 | 21 | q21.3 | listerin E3 ubiquitin protein ligase 1 [Source: HGNC Symbol; Acc: 13032] |
| 235306_at | GIMAP8 | ENSG00000171115 | 0.045949 | 1.1415232 | 7.866982 | 6.725458 | 7 | q36.1 | GTPase, IMAP family member 8 [Source: HGNC Symbol; Acc: 21792] |
| 213746_s_at | FLNA | ENSG00000196924 | 0.045983 | 0.6709764 | 10.55453 | 9.883554 | X | q28 | filamin A, alpha [Source: HGNC Symbol; Acc: 3754] |
| 200982_s_at | ANXA6 | ENSG00000197043 | 0.045983 | 0.9677565 | 9.852305 | 8.884549 | 5 | q33.1 | annexin A6 [Source: HGNC Symbol; Acc: 544] |
| 227029_at | FAM177A1 | ENSG00000151327 | 0.046043 | 0.8330088 | 6.455042 | 5.622033 | 14 | q13.2 | family with sequence similarity 177 member A1 [Source: HGNC |
| | | | | | | | | | Symbol; Acc: 19829] |
| 225695_at | SLC35F6 | ENSG00000213699 | 0.046225 | 0.7246104 | 9.024413 | 8.299803 | 2 | p23.3 | solute carrier family 35, member F6 [Source: HGNC Symbol; Acc: 26055] |
| 230263_s_at | DOCK5 | ENSG00000147459 | 0.046636 | 0.9841685 | 5.975225 | 4.991057 | 8 | p21.2 | dedicator of cytokinesis 5 [Source: HGNC Symbol; Acc: 23476] |
| 219860_at | LY6G5C | ENSG00000204428 | 0.04666 | 0.4988369 | 6.319773 | 5.820936 | 6 | p21.33 | lymphocyte antigen 6 complex, locus G5C [Source: HGNC Symbol; Acc: 13932] |
| 216620_s_at | ARHGEF10 | ENSG00000104728 | 0.04666 | 1.0059164 | 8.015577 | 7.009661 | 8 | p23.3 | Rho guanine nucleotide exchange factor (GEF) 10 [Source: HGNC |
| | | | | | | | | | Symbol; Acc: 14103] |
| 209298_s_at | ITSN1 | ENSG00000205726 | 0.046712 | 1.2772025 | 5.880818 | 4.603616 | 21 | q22.11 | intersectin 1 (SH3 domain protein) [Source: HGNC Symbol; Acc: 6183] |
| 219383_at | PRR5L | ENSG00000135362 | 0.046712 | 0.8741436 | 4.47531 | 3.601166 | 11 | p13 | proline rich 5 like [Source: HGNC Symbol; Acc: 25878] |
| 218204_s_at | FYCO1 | ENSG00000163820 | 0.046712 | 0.4753249 | 7.84106 | 7.365735 | 3 | p21.31 | FYVE and coiled-coil domain containing 1 [Source: HGNC Symbol; Acc: 14673] |
| 212637_s_at | WWP1 | ENSG00000123124 | 0.046712 | 0.9223494 | 7.411309 | 6.48896 | 8 | q21.3 | WW domain containing E3 ubiquitin protein ligase 1, [Source: HGNC |
| | | | | | | | | | Symbol; Acc: 17004] |
| 200784_s_at | LRP1 | ENSG00000123384 | 0.046712 | 0.8354694 | 10.71172 | 9.876255 | 12 | q13.3 | low density lipoprotein receptor-related protein 1 [Source: HGNC |
| | | | | | | | | | Symbol; Acc: 6692] |
| 202668_at | EFN82 | ENSG00000125266 | 0.046712 | 1.3006186 | 7.729295 | 6.428677 | 13 | q33.3 | ephrin-B2 [Source: HGNC Symbol; Acc: 3227] |
| 211926_s_at | MYH9 | ENSG00000100345 | 0.046851 | 0.5140278 | 10.37284 | 9.858809 | 22 | q12.3 | myosin, heavy chain 9, non-muscle [Source: HGNC Symbol; Acc: 7579] |
| 207181_s_at | CASP7 | ENSG00000165806 | 0.046856 | 0.8773186 | 8.172928 | 7.295609 | 10 | q25.3 | caspase 7, apoptosis-related cysteine peptidase (Source: HGNC Symbol; Acc: 1508] |
| 203940_s_at | VASH1 | ENSG00000071246 | 0.046904 | 0.759053 | 8.993567 | 8.234514 | 14 | q24.3 | vasohibin 1 [Source: HGNC Symbol; Acc: 19964] |
| 225046_at | LOC389831 | NA | 0.046904 | 1.209299 | 9.502744 | 8.293445 | NA | NA | NA |
| 229699_at | LOC1001295 | NA | 0.046904 | 0.7875228 | 7.252125 | 6.464602 | NA | NA | NA |
| | 50 | | | | | | | | |
| 1555997_s_at | IGFBP5 | ENSG00000115461 | 0.046904 | 2.0893027 | 9.039541 | 6.950238 | 2 | q36 | insulin-like growth factor binding protein 5 [Source: HGNC Symbol; Acc: 5474] |
| 221858_at | TBC1D12 | ENSG00000108239 | 0.046904 | 0.9754493 | 6.730089 | 5.75464 | 10 | q23.33 | TBC1 domain family, member 12 [Source: HGNC Symbol; Acc: 29082] |
| 202123_s_at | ABL1 | ENSG00000097007 | 0.046904 | 0.5359328 | 8.956464 | 8.420531 | 9 | q34.12 | c-abl oncogene 1, non-receptor tyrosine kinase (Source: HGNC Symbol; Acc: 76] |
| 209189_at | FOS | ENSG00000170345 | 0.046904 | 2.3113953 | 8.928569 | 6.617174 | 14 | q24.3 | FBJ murine osteosarcoma viral oncogene homolog (Source: HGNC |
| | | | | | | | | | Symbol; Acc: 3796] |
| 231697_s_at | VMP1 | ENSG00000062716 | 0.046904 | 1.3376767 | 9.338249 | 8.000572 | 17 | q23.1 | vacuole membrane protein 1 [Source: HGNC Symbol; Acc: 29559] |
| 231823_s_at | SH3PXD28 | ENSG00000174705 | 0.046907 | 1.3421906 | 8.749657 | 7.407467 | 5 | q35.1 | SH3 and PX domains 2B [Source: HGNC Symbol; Acc: 29242] |
| 226917_s_at | ANAPC4 | ENSG00000053900 | 0.046907 | 0.7157989 | 9.420943 | 8.705144 | 4 | p15.2 | anaphase promoting complex subunit 4 [Source: HGNC Symbol; Acc: 19990] |
| 213135_at | TIAM1 | ENSG00000156299 | 0.04696 | 1.432496 | 8.128731 | 6.696235 | 21 | q22.11 | T-cell lymphoma invasion and metastasis 1 [Source: HGNC Symbol; Acc: 11805] |
| 222793_at | DDX58 | ENSG00000107201 | 0.046977 | 0.7930309 | 6.897065 | 6.104034 | 9 | p21.1 | DEAD (Asp-Glu-Ala-Asp) box polypeptide 58 [Source: HGNC |
| | | | | | | | | | Symbol; Acc: 19102] |
| 235059_at | RAB12 | ENSG00000206418 | 0.047161 | 0.7361697 | 8.288455 | 7.552285 | 18 | p11.22 | RAB12, member RAS oncogene family [Source: HGNC Symbol; Acc: 31332] |
| 213364_s_at | SNX1 | ENSG00000028528 | 0.047358 | 0.9413216 | 7.632326 | 6.691004 | 15 | q22.31 | sorting nexin 1 [Source: HGNC Symbol,Acc: 11172] |
| 213194_at | ROBO1 | ENSG00000169855 | 0.047358 | 1.2023648 | 8.299204 | 7.09684 | 3 | p12.2 | roundabout, axon guidance receptor, homolog 1 (Drosophila) [Source: HGNC |
| | | | | | | | | | Symbol; Acc: 10249] |
| 223434_at | G6P3 | ENSG00000117226 | 0.047368 | 1.7670063 | 8.155929 | 6.388923 | 1 | p22.2 | guanylate binding protein 3 (Source: HGNC Symbol; Acc: 4184] |
| 201464_x_at | JUN | ENSG00000177606 | 0.047577 | 1.4116477 | 9.379206 | 7.967558 | 1 | p32.1 | jun proto-oncogene [Source: HGNC Symbol; Acc: 6204] |
| 228325_at | KIAA0146 | NA | 0.047577 | 1.6371158 | 6.878768 | 5.241652 | NA | NA | NA |
| 226639_at | SFT2D3 | ENSG00000173349 | 0.04777 | 0.4029701 | 7.472242 | 7.069272 | 2 | q14.3 | SFT2 domain containing 3 [Source: HGNC Symbol; Acc: 28767] |
| 204204_at | SLC31A2 | ENSG00000136867 | 0.047785 | 1.078147 | 7.858562 | 6.780415 | 9 | q32 | solute carrier family 31 (copper transporter), member 2 [Source: HGNC |
| | | | | | | | | | Symbol; Acc: 11017] |
| 213659_at | ZNF75D | ENSG00000186376 | 0.047785 | 0.6654956 | 7.859604 | 7.194108 | X | q26.3 | zinc finger protein 75D [Source: HGNC Symbol; Acc: 13145] |
| 219165_at | PDLIM2 | ENSG00000120913 | 0.047738 | 0.5378302 | 8.659829 | 8.121999 | 8 | p21.3 | PDZ and LIM domain 2 (mystique) [Source: HGNC Symbol; Acc: 13992] |
| 202704_at | TOB1 | ENSG00000141232 | 0.047841 | 1.1577332 | 7.864873 | 6.707139 | 17 | q21.33 | transducer of EREB2, 1 [Source: HGNC Symbol; Acc: 11979] |
| 201059_at | CTTN | ENSG00000085733 | 0.047841 | 1.13298 | 8.126829 | 6.993849 | 11 | q13.3 | cortactin [Source: HGNC Symbol; Acc: 3338] |
| 208626_s_at | VAT1 | ENSG00000108828 | 0.047843 | 0.9455257 | 10.23981 | 9.294287 | 17 | q21.31 | vesicle amine transport protein 1 homolog (T. californica) [Source: HGNC |
| | | | | | | | | | Symbol; Acc: 16919] |
| 224285_at | GPR174 | ENSG00000147138 | 0.047886 | 1.197457 | 7.961716 | 6.764259 | X | 821.1 | G protein-coupled receptor 174 [Source: HGNC Symbol; Acc: 30245] |
| 202746_at | ITM2A | ENSG00000078596 | 0.047974 | 1.7562969 | 8.555659 | 6.799362 | X | q21.1 | integral membrane protein 2A [Source: HGNC Symbol; Acc: 6173] |
| 1557749_at | EHBP1L1 | ENSG00000173442 | 0.047976 | 1.3008188 | 7.376098 | 6.075279 | 11 | q13.1 | EH domain binding protein 1-like 1 [Source: HGNC Symbol; Acc: 30682] |
| 208671_at | SERINC1 | ENSG00000111897 | 0.047976 | 0.9726095 | 8.57887 | 7.606261 | 6 | q22.31 | serine incorporator 1 [Source: HGNC Symbol; Acc: 13464] |
| 235199_at | RNF125 | ENSG00000101695 | 0.048039 | 1.0642075 | 7.065692 | 6.001485 | 18 | q12.1 | ring finger protein 125, E3 ubiquitin protein ligase [Source: HGNC |
| | | | | | | | | | Symbol; Acc: 21150] |
| 236565_s_dt | LARP6 | ENSG00000166173 | 0.048103 | 0.6843004 | 5.230392 | 4.546092 | 15 | q23 | La ribonucleoprotein domain family, member 5 [Source: HGNC |
| | | | | | | | | | Symbol; Acc: 24012] |
| 204575_s_at | MMP19 | ENSG00000123342 | 0.048165 | 0.946556 | 7.578627 | 6.632071 | 12 | q13.2 | matrix metallopeptidase 19 [Source: HGNC Symbol; Acc: 7165] |
| 221653_x_at | APOL2 | ENSG00000128335 | 0.048165 | 0.5885832 | 10.11617 | 9.527585 | 22 | q12.3 | apolipoprotein L, 2 [Source: HGNC Symbol; Acc: 619] |
| 224804_s_at | FAM219B | ENSG00000178761 | 0.048165 | 0.6515628 | 8.895176 | 8.243613 | 15 | q24.1 | family with sequence similarity 219, member B [Source: HGNC |
| | | | | | | | | | Symbol; Acc: 24695] |
| 202820_at | AHR | ENSG00000106546 | 0.048165 | 1.1914252 | 8.43326 | 7.241835 | 7 | p21.1 | aryl hydrocarbon receptor [Source: HGNC Symbol; Acc: 348] |
| 204082_at | PBX3 | ENSG00000167081 | 0.048165 | 0.6358091 | 8.576776 | 7.940967 | 9 | q33.3 | pre-B-cell leukemia homeobox 3 [Sourc.e: HGNC Symbol; Acc: 8634] |
| 218109_s_at | MFSD1 | ENSG00000118855 | 0.048305 | 1.1396733 | 10.13941 | 8.999736 | 3 | q25.32 | major facilitator superfamily domain containing 1 [Source: HGNC |
| | | | | | | | | | Symbol; Acc: 25874] |
| 231897_at | PTGR1 | ENSG00000106853 | 0.048305 | 1.1548912 | 7.789451 | 6.63456 | 9 | q31.3 | prostaglandin reductase 1 [Source: HGNC Symbol; Acc: 18429] |
| 205786_s_at | ITGAM | ENSG00000169896 | 0.048306 | 1.1378078 | 8.121972 | 6.984164 | 16 | p11.2 | integrin, alpha M (complement component 3 receptor 3 subunit) [Source: HGNC |
| | | | | | | | | | Symbol; Acc: 6149] |
| 203510_at | MET | ENSG00000105976 | 0.048306 | 1.6502092 | 7.842579 | 6.19237 | 7 | q31.2 | met proto-oncogene [Source: HGNC Symbol; Acc: 7029] |
| 224896_s_at | TTL | ENSG00000114999 | 0.048306 | 0.5603672 | 8.810442 | 8.250075 | 2 | q13 | tubulin tyrosine ligase [Source: HGNC Symbol; Acc: 21586] |
| 232645_at | LOC153684 | NA | 0.048306 | 0.888957 | 7.159878 | 6.270921 | NA | NA | NA |
| 226576_at | ARHGAP26 | ENSG00000145819 | 0.048306 | 0.8225882 | 6.112706 | 5.290117 | 5 | q31.3 | Rho GTPase activating protein 26 [Source: HGNC Symbol; Acc: 17073] |
| 219191_s_at | BIN2 | ENSG00000110934 | 0.048306 | 1.0978185 | 9.340657 | 8.242838 | 12 | q13.13 | bridging integrator 2 [Source: HGNC Symbol; Acc: 1053] |
| 214853_s_at | SHC1 | ENSG00000160691 | 0.048306 | 0.5806619 | 10.02587 | 9.445211 | 1 | q21.3 | SHC (Src homology 2 domain containing) transforming protein 1 [Source: HGNC |
| | | | | | | | | | Symbol; Acc: 10840] |
| 224358_s_at | MS4A7 | ENSG00000166927 | 0.048314 | 2.066881 | 8.506524 | 6.439643 | 11 | q12.2 | membrane-spanning 4-domains, subfamily A, member 7 [Source: HGNC |
| | | | | | | | | | Symbol; Acc: 13378] |
| 209164_s_at | CYB561 | ENSG00000008283 | 0.04856 | 0.8626733 | 8.334774 | 7.472101 | 17 | q23.3 | cytochrome b561 [Source: HGNC Symbol; Acc: 2571] |
| 222175_s_at | MED15 | ENSG00000099917 | 0.04856 | 0.4451901 | 9.6034 | 9.15821 | 22 | q11.21 | mediator complex subunit 15 [Source: HGNC Symbol; Acc: 14248] |
| 219469_at | DYNC2H1 | ENSG00000187240 | 0.04857 | 0.6595986 | 6.55585 | 5.896251 | 11 | q22.3 | dynein, cytoplasmic 2, heavy chain 1 [Source: HGNC Symbol; Acc: 2962] |
| 226143_at | RAI1 | ENSG00000108557 | 0.04857 | 1.2083934 | 8.541448 | 7.333054 | 17 | p11.2 | retinoic acid induced 1 [Source: HGNC Symbol; Acc: 9834] |
| 212681_at | EPB41L3 | ENSG00000082397 | 0.048619 | 1.3472919 | 8.071549 | 6.724257 | 18 | p11.31 | erythrocyte membrane protein band 4.1-like 3 [Source: HGNC Symbol; Acc: 3380] |
| 215784_at | CD1E | ENSG00000158488 | 0.04871 | 1.1453683 | 5.448261 | 4.302893 | 1 | q23.1 | CD1e molecule [Source: HGNC Symbol; Acc: 1638] |
| 211161_s_at | COL3A1 | ENSG00000168542 | 0.04871 | 2.0294663 | 11.58326 | 9.553789 | 2 | q32.2 | collagen, type III, alpha 1 [Source: HGNC Symbol; Acc: 2201] |
| 209906_at | C3AR1 | ENSG00000171860 | 0.04881 | 1.1815832 | 9.984971 | 8.803387 | 12 | p13.31 | complement component 3a receptor 1 [Source: HGNC Symbol; Acc: 1319] |
| 200625_s_at | CAP1 | ENSG00000131236 | 0.048952 | 0.8385932 | 11.78723 | 10.94863 | 1 | p34.2 | CAP, adenylate cyclase-associated protein 1 (yeast) [Source: HGNC |
| | | | | | | | | | Symbol; Acc: 20040] |
| 209550_at | NDN | ENSG00000182636 | 0.048952 | 0.8892051 | 8.585921 | 7.696716 | 15 | q11.2 | necdin, melanoma antigen (MAGE) family member [Source: HGNC |
| | | | | | | | | | Symbol; Acc: 7675] |
| 214039_s_at | LAPTM4B | ENSG00000104341 | 0.048974 | 1.6628259 | 7.882224 | 6.219398 | 8 | q22.1 | lysosomal protein transmembrane 4 beta [Source: HGNC Symbol; Acc: 13646] |
| 209356_x_at | EFEMP2 | ENSG00000172638 | 0.049064 | 0.7587052 | 8.007888 | 7.249183 | 11 | q13.1 | EGF containing fibulin-like extracellular matrix protein 2 [Source: HGNC |
| | | | | | | | | | Symbol; Acc: 3219] |
| 207276_at | CDR1 | ENSG00000181258 | 0.049178 | 1.8925946 | 9.606612 | 7.714017 | X | q27.1 | cerebellar degeneration-related protein 1, 34 kDa [Source: HGNC |
| | | | | | | | | | Symbol; Acc: 1798] |
| 209955_s_at | FAP | ENSG00000078098 | 0.049178 | 1.4777063 | 8.251596 | 6.77389 | 2 | q24.2 | fibroblast activation protein, alpha [Source: HGNC Symbol; Acc: 3590] |
| 226844_at | MOB38 | ENSG00000120162 | 0.049203 | 1.1637648 | 6.92211 | 5.758345 | 9 | p21.2 | MOB kinase activator 3B [Source: HGNC Symbol; Acc: 23825] |
| 222468_at | KIAA0319L | ENSG00000142687 | 0.049203 | 0.3011724 | 8.203036 | 7.901864 | 1 | p34.3 | KIAA0319-like [Source: HGNC Symbol; Acc: 30071] |
| 226056_at | ARHGAP31 | ENSG00000031081 | 0.049247 | 0.7875162 | 8.433132 | 7.645616 | 3 | q13.33 | Rho GTPase activating protein 31 [Source: HGNC Symbol; Acc: 29216] |
| 226695_at | PRRX1 | ENSG00000116132 | 0.049316 | 1.7627347 | 9.80378 | 8.041045 | 1 | q24.2 | paired related homeobox 1 [Source: HGNC Symbol; Acc: 9142] |
| 204844_at | ENPEP | ENSG00000138792 | 0.049442 | 0.6595157 | 4.491515 | 3.831999 | 4 | q25 | glutamyl aminopeptidase(aminopeptidase A) [Source: HGNC Symbol; Acc: 3355] |
| 200612_s_at | AP2B1 | ENSG00000006125 | 0.049442 | 0.5957471 | 8.555796 | 7.960049 | 17 | q12 | adaptor-related protein complex 2, beta 1 subunit [Source: HGNC Symbol; Acc: 563] |
| 200923_at | LGALS3BP | ENSG00000108679 | 0.049442 | 0.9211334 | 10.07362 | 9.152489 | 17 | q25.3 | lectin, galactoside-binding, soluble, 3 binding protein [Source: HGNC |
| | | | | | | | | | Symbol; Acc: 6564] |
| 208074_s_at | AP2S1 | ENSG00000042753 | 0.049593 | 0.5957933 | 10.51239 | 9.916594 | 19 | q13.32 | adaptor-related protein complex 2, sigma 1 subunit [Source: HGNC |
| | | | | | | | | | Symbol; Acc: 565] |
| 200897_s_at | PALLD | ENSG00000129116 | 0.049599 | 1.6018945 | 9.526129 | 7.924234 | 4 | q32.3 | palladin, cytoskeletal associated protein [Source: HGNC Symbol; Acc: 17068] |
| 209667_at | CES2 | ENSG00000172831 | 0.049599 | 0.5127909 | 8.055154 | 7.542363 | 16 | q22.1 | carboxylesterase 2 [Source: HGNC. Symbol; Acc: 1864] |
| 225785_at | REEP3 | ENSG00000165476 | 0.049649 | 1.1129933 | 7.249649 | 6.136656 | 10 | q21.3 | receptor accessory protein 3 [Source: HGNC Symbol; Acc: 23711] |
| 227265_at | FGL2 | ENSG00000127951 | 0.049649 | 1.7680217 | 9.464203 | 7.696181 | 7 | q11.23 | fibrinogen-like 2 [Source: HGNC Symbol; Acc: 3696] |
| 226679_at | SLC26A11 | ENSG00000181045 | 0.049649 | 1.3774827 | 8.62206 | 7.244577 | 17 | q25.3 | solute carrier family 26, member 11 [Source: HGNC Symbol; Acc: 14471] |
| 202441_at | ERLIN1 | ENSG00000107566 | 0.049691 | 0.5854142 | 8.619248 | 8.033834 | 10 | q24.31 | ER lipid raft associated 1 [Source: HGNC Symbol; Acc: 16947] |
| 227758_at | RERG | ENSG00000134533 | 0.049698 | 1.1309377 | 5.789853 | 4.658915 | 12 | p12.3 | RAS-like, estrogen-regulated, growth inhibitor [Source: HGNC Symbol; Acc: 15980] |
| 204036_at | LPAR1 | ENSG00000198121 | 0.049814 | 1.4440394 | 6.674595 | 5.230556 | 9 | q31.3 | lysophosphatidic acid receptor 1 [Source: HGNC Symbol; Acc: 3166] |
| 225755_at | KLHDC8B | ENSG00000185909 | 0.049849 | 0.8512009 | 8.005692 | 7.154491 | 3 | p21.31 | kelch domain containing 8B [Source: HGNC Symbol; Acc: 28557] |
| 209571_at | CIR1 | ENSG00000138433 | 0.049849 | 0.3762687 | 8.299113 | 7.922845 | 2 | q31.1 | corepressor interacting with RBPJ, 1 [Source: HGNC Symbol; Acc: 24217] |
| 210184_at | ITGAX | ENSG00000140678 | 0.049853 | 1.1127795 | 8.872884 | 7.760104 | 16 | p11.2 | integrin, alpha X (complement component 3 receptor 4 subunit) [Source: HGNC |
| | | | | | | | | | Symbol; Acc: 6152] |
| 217940_s_at | CARKD | ENSG00000213995 | 0.049853 | 0.4806867 | 8.908728 | 8.428041 | 13 | q34 | carbohydrate kinase domain containing [Source: HGNC Symbol; Acc: 25576] |
| 212993_at | NACC2 | ENSG00000148411 | 0.050115 | 1.1149639 | 7.888032 | 6.773068 | 9 | q34.3 | NACC family member 2, BEN and BTB (POZ) domain containing [Source: HGNC |
| | | | | | | | | | Symbol; Acc: 23846] |
| 1562876_s_at | LOC541471 | NA | 0.050218 | 0.6825624 | 3.603678 | 2.921115 | NA | NA | NA |
| 242268_at | CELF2 | ENSG00000048740 | 0.050218 | 1.5536681 | 7.210053 | 5.656385 | 10 | p14 | CNC: BP, Elav-like family member 2 [Source: HGNC Symbol; Acc: 2550] |
| 213125_at | OLFML2B | ENSG00000162745 | 0.050218 | 0.7874654 | 8.741576 | 7.95411 | 1 | q23.3 | olfactomedin-like 2B [Source: HGNC Symbol; Acc: 24558] |
| 221257_x_at | FBXO38 | ENSG00000145868 | 0.050301 | 0.5976252 | 7.698195 | 7.10057 | 5 | q32 | F-box protein 38 [Source: HGNC Symbol; Acc: 28844] |
| 236782_at | SAMD3 | ENSG00000164483 | 0.0504 | 1.0727407 | 7.59099 | 6.51825 | 6 | q23.1 | sterile alpha motif domain containing 3 [Source: HGNC Symbol; Acc: 21574] |
| 1554690_a_at | TACC1 | ENSG00000147526 | 0.050506 | 1.079368 | 9.700335 | 8.620967 | 8 | p11.22 | transforming, acidic coiled-coil containing protein 1 [Source: HGNC |
| | | | | | | | | | Symbol; Acc: 11522] |
| 203431_s_at | ARHGAP32 | ENSG00000134909 | 0.050519 | 1.2383399 | 6.793821 | 5.555481 | 11 | q24.3 | Rho GTPase activating protein 32 [Source: HGNC Symbol; Acc: 17399] |
| 226711_at | FOXN2 | ENSG00000170802 | 0.050636 | 0.9371015 | 9.838676 | 8.901574 | 2 | p16.3 | forkhead box N2 [Source: HGNC SymbOL; Acc: 5281] |
| 203562_at | FEZ1 | ENSG00000149557 | 0.050636 | 1.4246364 | 7.169091 | 5.744454 | 11 | q24.2 | fasciculation and elongation protein zeta 1 (zygin I) [Source: HGNC |
| | | | | | | | | | Symbol; Acc: 36591 |
| 201508_at | IGFBP4 | ENSG00000141753 | 0.050636 | 1.2181533 | 10.95048 | 9.732328 | 17 | q21.2 | insulin-like growth factor binding protein 4 [Source: HGNC Symbol; Acc: 5473] |
| 209933_s_at | CD300A | ENSG00000167851 | 0.050888 | 1.0639508 | 8.6536 | 7.589649 | 17 | q25.1 | CD300a molecule [Source: HGNC Symbol; Acc: 19319] |
| 226751_at | CNRIP1 | ENSG00000119865 | 0.050983 | 1.0463666 | 7.446207 | 6.39984 | 2 | p14 | cannabinoid receptor interacting protein 1 [Source: HGNC Symbol; Acc: 24546] |
| 203303_at | DYNLT3 | ENSG00000165169 | 0.050983 | 0.8389781 | 9.479702 | 8.640724 | X | p11.4 | dynein, light chain, Tctex-type 3 [Source: HGNC Symbol; Acc: 11694] |
| 1556698_a_at | GPRIN3 | ENSG00000185477 | 0.050983 | 1.2153376 | 6.331782 | 5.116444 | 4 | q22.1 | GPRIN family member 3 [Source: HGNC Symbol; Acc: 27733] |
| 223454_at | CXCL16 | ENSG00000161921 | 0.050983 | 0.9870947 | 9.755065 | 3.76797 | 17 | p13.2 | chemokine (C-X-C motif) ligand 16 [Source: HGNC Symbol; Acc: 16642] |
| 20203_s_at | MAN2A2 | ENSG00000196547 | 0.050983 | 0.8858919 | 9.564687 | 8.678795 | 15 | q26.1 | mannosidase, alpha, class 2A, member 2 [Source: HGNC Symbol Acc: 6825] |
| 205779_at | RAMP2 | ENSG00000131477 | 0.051156 | 0.9338225 | 8.483655 | 7.549832 | 17 | q21.31 | receptor (G protein-coupled) activity modifying protein 2 [Source: HGNC |
| | | | | | | | | | Symbol; Acc: 9844] |
| 209110_s_at | RGL2 | ENSG00000237441 | 0.051156 | 0.6625429 | 8.743822 | 8.081279 | 6 | p21.32 | ral guanine nucleotide dissociation stimulator-like 2 [Source: HGNC |
| | | | | | | | | | Symbol; Acc: 9769] |
| 209935_at | ATP2C1 | ENSG00000017260 | 0.051156 | 0.6570157 | 6.368275 | 5.71126 | 3 | q22.1 | ATPase, Ca [symbol] transporting, type 2C, member 1 [Source: HGNC |
| | | | | | | | | | Symbol; Acc: 13211] |
| 214453_s_at | IFI44 | ENSG00000137965 | 0.051156 | 1.6331853 | 9.486684 | 7.853498 | 1 | p31.1 | interferon-induced protein 44 [Source: HGNC Symbol; Acc: 16938] |
| 201063_at | RCN1 | ENSG00000049449 | 0.051233 | 0.7028097 | 9.216223 | 8.513413 | 11 | p13 | reticulocalbin 1, EF-hand calcium binding domain [Source: HGNC |
| | | | | | | | | | Symbol; Acc: 9934] |
| 213222_at | PLCB1 | ENSG00000182621 | 0.051361 | 0.7004754 | 4.53598 | 3.835504 | 20 | p12.3 | phospholipase C, beta 1 (phosphoinositide-specific) [Source: HGNC |
| | | | | | | | | | Symbol; Acc: 15917] |
| 201069_at | MMP2 | ENSG00000087245 | 0.051361 | 1.6913538 | 10.01928 | 8.32793 | 16 | q12.2 | matrix matallopeptidase 2 (gelatinase A, 72 kDa gelatinase, 72 kDa type IV |
| | | | | | | | | | collagenase) [Source: HGNC Symbol; Acc: 7166] |
| 200661_at | CTSA | ENSG00000064601 | 0.051361 | 0.720607 | 10.64337 | 9.922764 | 20 | q13.12 | cathepsin A [Source: HGNC Symbol; Acc: 9251] |
| 213258_at | TFPI | ENSG00000003436 | 0.051379 | 1.4194378 | 8.02985 | 6.610412 | 2 | q32.1 | tissue factor pathway inhibitor (lipoprotein-associated coagulation inhibitor) |
| | | | | | | | | | [Source: HGNC Symbol; Acc: 11760] |
| 225414_at | RNF149 | ENSG00000163162 | 0.051422 | 0.8046721 | 9.11227 | 8.307597 | 2 | q11.2 | ring finger protein 149 [Source: HGNC Symbol; Acc: 23137] |
| 219397_at | COQ10B | ENSG00000115520 | 0.051422 | 0.6544798 | 7.359952 | 6.705472 | 2 | q33.1 | coenzyme Q10 homolog B (S. cerevisiae) [Source: HGNC Symbol; Acc: 25819] |
| 222127_s_at | EXOC1 | ENSG00000090989 | 0.051603 | 0.7543635 | 9.746304 | 8.99194 | 4 | q12 | exocyst complex component 1 [Source: HGNC Symbol; Acc: 30380] |
| 223220_s_at | PARP9 | ENSG00000138496 | 0.051603 | 0.9799493 | 9.260982 | 8.281032 | 3 | q21.1 | poly (ADP-ribose) polymerase family, member 9 [Source: HGNC |
| | | | | | | | | | Symbol; Acc: 24118] |
| 213422_s_at | MXRA8 | ENSG00000162576 | 0.051603 | 1.2422437 | 8.999253 | 7.75701 | 1 | p36.33 | matrix-remodelling associated 8 [Source: HGNC Symbol; Acc: 7542] |
| 203153_at | IFIT1 | ENSG00000185745 | 0.051753 | 1.5289266 | 7.719972 | 6.191046 | 10 | q23.31 | interferon-induced protein with tetratricopeptide repeats 1 [Source: HGNC |
| | | | | | | | | | Symbol; Acc: 5407] |
| 202100_at | RALB | ENSG00000144118 | 0.051843 | 0.6714025 | 8.880756 | 8.209353 | 2 | q14.2 | v-ral simian leukemia viral oncogene homolog B [Source: HGNC |
| | | | | | | | | | Symbol; Acc: 9840] |
| 224895_at | VAP1 | ENSG00000137693 | 0.051946 | 1.4909459 | 7.474063 | 5.983117 | 11 | q22.1 | Yes-associated protein 1 [Source: HGNC Symbol; Acc: 16262] |
| 201368_at | ZFP36L2 | ENSG00000152518 | 0.052319 | 0.9647118 | 10.56869 | 9.603976 | 2 | p21 | ZFP36 ring finger protein-like 2 [Source: HGNC Symbol; Acc: 1108] |
| 212027_at | RBM25 | ENSG00000119707 | 0.052384 | 0.6781906 | 9.494824 | 8.816633 | 14 | q24.2 | RNA binding motif protein 25 [Source: HGNC Symbol; Acc: 23244] |
| 221942_s_at | GUCY1A3 | ENSG00000164116 | 0.05244 | 1.3677707 | 7.987539 | 6.619768 | 4 | q32.1 | guanylate cyclase 1, soluble, alpha 3 [Source: HGNC Symbol; Acc: 46 ] |
| 213800_at | CFH | ENSG00000000971 | 0.05244 | 1.2322861 | 7.757237 | 6.524951 | 1 | q31.3 | complement factor H [Source: HGNC Symbol; Acc: 4883] |
| 1554999_at | RASGEF1B | ENSG00000138670 | 0.052614 | 1.4173975 | 6.341497 | 4.9241 | 4 | q21.22 | RasGEF domain family, member 1B [Source: HGNC Symbol; Acc: 24881] |
| 218606_at | ZDHHC7 | ENSG00000153786 | 0.052737 | 0.3905379 | 8.928964 | 8.538426 | 16 | q24.1 | zinc finger, DHHC-type containing 7 [Source: HGNC Symbol; Acc: 18459] |
| 204083_s_at | TPM2 | ENSG00000198467 | 0.052794 | 1.005641 | 10.06504 | 9.059402 | 9 | p13.3 | tropomyosin 2 (beta) [Source: HGNC Symbol; Acc: 12011] |
| 1553955_at | PPP1R21 | ENSG00000162869 | 0.052933 | 0.8388302 | 8.37991 | 7.54108 | 2 | p16.3 | protein phosphatase 1, regulatory subunit 21 [Source: HGNC Symbol; Acc: 30595] |
| 228083_at | CACNA2D4 | ENSG00000151062 | 0.052933 | 0.833099 | 7.252949 | 6.41985 | 12 | p13.33 | calcium channel, voltage-dependent, alpha 2/delta subunit 4 [Source: HGNC |
| | | | | | | | | | Symbol; Acc: 20202] |
| 227304_at | SMCR8 | ENSG00000176994 | 0.052933 | 0.7999914 | 8.377827 | 7.577836 | 17 | p11.2 | Smith-Magenis syndrome chromosome region, candidate 8 [Source: HGNC |
| | | | | | | | | | Symbol; Acc: 17921] |
| 225710_at | GNB4 | ENSG00000114450 | 0.052933 | 1.0875404 | 8.393035 | 7.305494 | 3 | q26.33 | guanine nucleotide binding protein (G protein), beta polypeptide 4 [Source: HGNC |
| | | | | | | | | | Symbol; Acc: 20731] |
| 204154_at | CDO1 | ENSG00000129596 | 0.052989 | 1.1929186 | 5.120926 | 3.928008 | 5 | q22.3 | cysteine dioxygenase type 1 [Source: HGNC Symbol; Acc: 1795] |
| 228318_s_at | CRIPAK | ENSG00000179979 | 0.053008 | 0.5311063 | 6.682491 | 6.151384 | 4 | p16.3 | cysteine-rich PAK1 inhibitor [Source: HGNC Symbol; Acc: 26619] |
| 227013_at | LATS2 | ENSG00000150457 | 0.053008 | 1.1010683 | 7.793207 | 6.692139 | 13 | q12.11 | large tumor suppressor kinase 2 [Source: HGNC Symbol; Acc: 6515] |
| 1558692_at | C1orf85 | ENSG00000198715 | 0.05311 | 0.572439 | 5.400512 | 4.828073 | 1 | q22 | chromosome 1 open reading frame 85 [Source: HGNC Symbol; Acc: 29436] |
| 213238_at | ATP10D | ENSG00000145246 | 0.05311 | 1.0510381 | 8.61573 | 7.564692 | 4 | p12 | ATPase, class V, type 10D [Source: HGNC Symbol; Acc: 13549] |
| 201850_at | CAPG | ENSG00000042493 | 0.053135 | 0.8093839 | 10.7541 | 9.944719 | 2 | p11.2 | capping protein (actin filament), gelsolin-like [Source: HGNC Symbol; Acc: 1474] |
| 209717_at | EVIS | ENSG00000067208 | 0.05314 | 0.7114496 | 7.081763 | 6.370314 | 1 | p22.1 | ecotropic viral integration site 5 [Source: HGNC Symbol; Acc: 3501] |
| 227388_at | TUSC1 | ENSG00000198680 | 0.053261 | 1.5643673 | 8.139273 | 6.574905 | 9 | p21.2 | tumor suppressor candidate 1 [Source: HGNC Symbol; Acc: 31010] |
| 200771_at | LAMC1 | ENSG00000135862 | 0.053546 | 0.8858041 | 8.418292 | 7.532487 | 1 | q25.3 | laminin, gamma 1 (formerly LAMB2) [Source: HGNC Symbol; Acc: 6492] |
| 229055_at | GPR68 | ENSG00000119714 | 0.053546 | 0.8377005 | 9.226091 | 8.38839 | 14 | q32.11 | G protein-coupled receptor 68 [Source: HGNC Symbol; Acc: 4519] |
| 200927_s_at | RAB14 | ENSG00000119396 | 0.053624 | 0.5226401 | 8.220466 | 7.697826 | 9 | q33.2 | RAB14, member RAS oncogene family [Source: HGNC Symbol; Acc: 16524] |
| 225447_at | GPD2 | ENSG00000115159 | 0.053524 | 0.9677597 | 8.485215 | 7.517456 | 2 | q24.1 | glycerol-3-phosphate dehydrogenase 2 (mitochondrial) [Source: HGNC |
| | | | | | | | | | Symbol; Acc: 4456] |
| 225525_at | KIAA1671 | ENSG00000197077 | 0.0538 | 1.4467482 | 7.312948 | 5.866199 | 22 | q11.23 | KIAA1671 [Source: HGNC Symbol; Acc: 29345] |
| 209649_at | STAM2 | ENSG00000115145 | 0.053804 | 0.7574983 | 7.629712 | 6.872214 | 2 | q23.3 | signal transducing adaptor molecule (SH3 domain and ITAM motif) 2 |
| | | | | | | | | | [Source: HGNC Symbol; Acc: 11358] |
| 219492_at | CHIC2 | ENSG00000109220 | 0.054092 | 0.6729025 | 8.386462 | 7.713559 | 4 | q12 | cysteine-rich hydrophobic domain 2 [Source: HGNC Symbol; Acc: 1935] |
| 1560060_s_at | VPS37C | ENSG00000167987 | 0.054133 | 0.5575703 | 8.463034 | 7.905464 | 11 | q12.2 | vacuolar protein sorting 37 homolog C (S. cerevisiae) [Source: HGNC |
| | | | | | | | | | Symbol; Acc: 26097] |
| 217947_at | CMTM6 | ENSG00000091317 | 0.054172 | 0.7023048 | 10.47748 | 9.775178 | 3 | p22.3 | CKLF-like MARVEL transmembrane domain containing 6 [Source: HGNC |
| | | | | | | | | | Symbol; Acc: 19177] |
| 219078_at | GPATCH2 | ENSG00000092978 | 0.054197 | 0.9518562 | 7.045736 | 6.09388 | 1 | q41 | G patch domain containing 2 [Source: HGNC Symbol; Acc: 25499] |
| 203854_at | CFI | ENSG00000205403 | 0.054292 | 1.2057395 | 6.704059 | 5.49832 | 4 | q25 | complement factor 1 [Source: HGNC Symbol; Acc: 5394] |
| 209637_s_at | RGS12 | ENSG00000159788 | 0.054328 | 0.5021003 | 7.515978 | 7.013877 | 4 | p16.3 | regulator of G-protein signaling 12 [Source: HGNC Symbol; Acc: 9994] |
| 202252_at | RAB13 | ENSG00000143545 | 0.05435 | 0.9137575 | 9.579586 | 8.665828 | 1 | q21.3 | RAB13, member RAS oncogene family [Source: HGNC Symbol; Acc: 9762] |
| 227373_at | ATXN1L | ENSG00000224470 | 0.05435 | 0.7597165 | 8.404084 | 7.644368 | 16 | q22.2 | ataxin 1-like [Source: HGNC Symbol; Acc: 33279] |
| 207469_s_at | PIR | ENSG00000087842 | 0.05446 | 0.663779 | 6.265059 | 5.60128 | X | p22.2 | pirin (iron-binding nuclear protein) [Source: HGNC Symbol; Acc: 30048] |
| 212543_at | AIM1 | ENSG00000112297 | 0.054464 | 0.7881191 | 9.68228 | 8.894161 | 6 | q21 | absent in melanoma 1 [Source: HGNC Symbol; Acc: 356] |
| 208983_s_at | PECAM1 | NA | 0.054524 | 1.305077 | 10.69254 | 9.387459 | NA | NA | NA |
| 226438_at | SNTB1 | ENSG00000172164 | 0.054649 | 0.8096096 | 6.953942 | 6.144333 | 8 | q24.12 | syntrophin, beta 1 (dystrophin-associated protein A1, 59 kDa, basic component 1) |
| | | | | | | | | | [Source: HGNC Symbol; Acc: 11168] |
| 201567_s_at | GOLGA4 | ENSG00000144674 | 0.054737 | 0.5440613 | 8.834092 | 8.29003 | 3 | p22.2 | golgin A4 [Source: HGNC Symbol; Acc: 4427] |
| 204520_x_at | BRD1 | ENSG00000100425 | 0.054846 | 0.4383699 | 9.00874 | 8.57037 | 22 | q13.33 | bromodomain containing 1 [Source: HGNC Symbol; Acc: 1102] |
| 217828_at | SLTM | ENSG00000137776 | 0.054846 | 0.8307026 | 8.864809 | 8.034107 | 15 | q22.1 | SAFB-like; transcription modulator [Source: HGNC Symbol; Acc: 20709] |
| 224689_at | MANBAL | ENSG00000101363 | 0.054846 | 0.3402156 | 9.457076 | 9.11686 | 20 | q11.23 | mannosidese, beta A, lysosomal-like [Source: HGNC Symbol; Acc: 15799] |
| 219157_at | KLHL2 | ENSG00000109466 | 0.054893 | 0.6367528 | 8.271661 | 7.634908 | 4 | q32.3 | kelch-like family member 2 [Source: HGNC Symbol; Acc: 6353] |
| 232024_at | GIMAP2 | ENSG00000105560 | 0.05502 | 1.1621322 | 7.593743 | 6.431611 | 7 | q36.1 | GTPase, IMAP family member 2 [Source: HGNC Symbol; Acc: 21789] |
| 219037_at | ASPN | ENSG00000106819 | 0.055081 | 1.8444311 | 7.912112 | 6.067681 | 9 | q22.31 | asporin [Source: HGNC Symbol; Acc: 14872] |
| 213010_at | PRKCDBP | ENSG00000170955 | 0.055081 | 0.6667374 | 7.157466 | 6.490729 | 11 | p15.4 | protein kinase C, delta binding protein [Source: HGNC Symbol; Acc: 9400] |
| 219570_at | KIF16B | ENSG00000089177 | 0.055081 | 0.9395247 | 6.792996 | 5.853471 | 20 | p12.1 | kinesin family member 16B [Source: HGNC Symbol; Acc.15869] |
| 214066_x_at | NPR2 | ENSG00000159899 | 0.055081 | 0.4995605 | 7.871515 | 7.371954 | 9 | p13.3 | natriuretic peptide receptor B/guanylate cyclase B (atrionatriuretic peptide |
| | | | | | | | | | receptor B) [Source: HGNC Symbol; Acc: 7944] |
| 228372_at | C10orf128 | ENSG00000204161 | 0.055081 | 1.2282534 | 8.331788 | 7.103535 | 10 | q11.23 | chromosome 10 open reading frame 128 [Source: HGNC Symbol; Acc: 27274] |
| 230276_at | FAM49A | ENSG00000197872 | 0.055081 | 1.2864889 | 6.31294 | 5.026451 | 2 | p24.2 | family with sequence similarity 49, member A [Source: HGNC Symbol; Acc: 25373] |
| 203002_at | AMOTL2 | ENSG00000114019 | 0.055081 | 1.3811705 | 7.607053 | 6.225883 | 3 | q22.2 | angiomotin like 2 [Source: HGNC Symbol; Acc: 17812] |
| 225688_s_at | PHLDB2 | ENSG00000144824 | 0.055081 | 1.6421107 | 7.415402 | 5.773291 | 3 | q13.2 | pleckstrin homology-like domain, family B, member 2 [Source: HGNC |
| | | | | | | | | | Symbol; Acc: 29573] |
| 1553678_a_at | ITGB1 | ENSG00000150093 | 0.055081 | 0.7623281 | 9.604847 | 8.842519 | 10 | p11.22 | integrin, beta 1 (fibronectin receptor, beta polypeptide, antigen CD29 includes |
| | | | | | | | | | MDF2, MSK12) [Source: HGNC Symbol; Acc: 6153] |
| 203476_at | TPBG | ENSG00000145242 | 0.055081 | 1.4028702 | 8.397859 | 6.994989 | 6 | q14.1 | trophoblast glycoprotein [Source: HGNC Symbol; Acc: 12004] |
| 201180_s_at | GNAI3 | ENSG00000065135 | 0.055081 | 0.7164849 | 9.753543 | 9.037058 | 1 | p13.3 | guanine nucleotide binding protein (G protein), alpha nhibiting activity |
| | | | | | | | | | polypeptide 3 [Source: HGNC Symbol; Acc: 4387] |
| 220141_at | C11orf63 | ENSG00000109944 | 0.055081 | 0.5302891 | 5.631726 | 5.101437 | 11 | q24.1 | chromosome 11 open reading frame 63 [Source: HGNC Symbol; Acc: 26283] |
| 227923_at | SHANK3 | ENSG00000251322 | 0.055081 | 1.1756261 | 7.738818 | 6.563191 | 22 | q13.33 | SH3 and multiple ankyrin repeat domains 3 [Source: HGNC Symbol; Acc: 14294] |
| 235334_at | ST6GALNAC3 | ENSG00000184005 | 0.055081 | 0.8325709 | 5.744061 | 4.91149 | 1 | p31.1 | ST6 (alpha-N-acetyl-neuraminyl-2,3-beta-galactosyl-1,3)-N-acetylgalactosaminide |
| | | | | | | | | | alpha-2,6-sialyltransferase 3 [Source: HGNC Symbol; Acc: 19343] |
| 235411_at | PGBD1 | ENSG00000137338 | 0.055081 | 0.4549063 | 5.741778 | 5.286872 | 6 | p22.1 | piggyBac transposable element derived 1 [Source: HGNC Symbol; Acc: 19398] |
| 216689_x_at | ARHGAP1 | ENSG00000175220 | 0.055086 | 0.5210108 | 9.747526 | 9.226515 | 11 | p11.2 | Rho GTPase activating protein 1 [Source: HGNC Symbol; Acc: 673] |
| 202932_at | YES1 | ENSG00000176105 | 0.055086 | 1.2124045 | 6.25973 | 5.047325 | 18 | p11.32 | v-yes-1 Yamaguchi sarcoma viral oncogene homolog 1 [Source: HGNC |
| | | | | | | | | | Symbol; Acc: 12841] |
| 212293_at | HIPK1 | ENSG00000163349 | 0.055095 | 0.6908166 | 7.914422 | 7.223606 | 1 | p13.2 | homeodomain interacting protein kinase 1 [Source: HGNC Symbol; Acc: 19006] |
| 225618_at | ARHGAP27 | ENSG00000159314 | 0.055334 | 0.4730209 | 7.935837 | 7.462816 | 17 | q21.31 | Rho GTPase activating protein 27 [Source: HGNC Symbol; Acc: 31813] |
| 201242_s_at | ATP1B1 | ENSG00000143153 | 0.055334 | 1.2831495 | 7.126474 | 5.843325 | 1 | q24.2 | ATPase, Na>w [symbol] transporting, beta 1 polypeptide [Source: HGNC |
| | | | | | | | | | Symbol; Acc: 804] |
| 203583_at | UNC50 | ENSG00000115446 | 0.055334 | 0.4527831 | 8.989706 | 8.536923 | 2 | q11.2 | unc-50 homolog (C. elegans) [Source: HGNC Symbol; Acc: 16046] |
| 1557938_s_at | PTRF | ENSG00000177469 | 0.055334 | 0.8943521 | 8.2614 | 7.367048 | 17 | q21.2 | polymerase land transcript release factor [Source: HGNC Symbol; Acc: 9688] |
| 242321_at | PTPN14 | ENSG00000152104 | 0.055342 | 1.3269817 | 5.787995 | 4.461014 | 1 | q41 | protein tyrosine phosphatase, non-receptor type 14 [Source: HGNC |
| | | | | | | | | | Symbol; Acc: 9647] |
| 212950_at | GPR116 | ENSG00000069122 | 0.055342 | 1.4662875 | 6.762014 | 5.295727 | 6 | p12.3 | G protein-coupled receptor 116 [Source: HGNC Symbol; Acc: 19030] |
| 205639_at | AOAH | ENSG00000136250 | 0.055342 | 0.6545201 | 9.612123 | 8.957603 | 7 | p14.2 | acyloxyacyl hydrolase (neutrophil) [Source: HGNC Symbol; Acc: 543] |
| 223209_s_at | VIMP | ENSG00000131871 | 0.055342 | 0.8381324 | 9.523564 | 8.685432 | 15 | q26.3 | VCP-interacting membrane protein [Source: HGNC Symbol; Acc: 30396] |
| 208131_s_at | PTGIS | ENSG00000124212 | 0.055389 | 1.3212 | 7.49094 | 6.16974 | 20 | q13.13 | prostaglandin 12 (prostacyclin) synthase [Source: HGNC Symbol; Acc: 9603] |
| 227833_s_at | MBD6 | ENSG00000166987 | 0.055397 | 0.7519226 | 10.5182 | 9.766277 | 12 | q13.3 | methyl-CpG binding domain protein 6 [Source: HGNC Symbol; Acc: 20445] |
| 208991_at | STAT3 | ENSG00000168610 | 0.055397 | 0.946587 | 9.530425 | 8.583838 | 17 | q21.2 | signal transducer and activator of transcription 3 (acute-phase response factor) |
| | | | | | | | | | [Source: HGNC Symbol; Acc: 11364] |
| 216361_s_at | KAT6A | ENSG00000083168 | 0.055397 | 0.6517457 | 7.927872 | 7.276126 | 8 | p11.21 | K(lysine) acetyltransferase 6A [Source: HGNC Symbol; Acc: 13013] |
| 1554106_at | NBEAL1 | ENSG00000144426 | 0.055397 | 0.6619161 | 7.462248 | 6.800332 | 2 | q33.2 | neurobeachin-like 1 [Source: HGNC Symbol; Acc: 20631] |
| 209357_at | CITED2 | ENSG00000164442 | 0.055397 | 1.2323825 | 9.303495 | 8.071112 | 6 | q24.1 | Cbp/p300-interacting transactivator, with Glu/Asp-rich carboxy-terminal domain, |
| | | | | | | | | | 2 [Source: HGNC Symbol; Acc: 1987] |
| 53720_at | C19orf66 | ENSG00000130813 | 0.055432 | 0.9673751 | 9.679982 | 8.712607 | 19 | p13.2 | chromosome 19 open reading frame 66 [Source: HGNC Symbol; Acc: 25649] |
| 228964_at | PRDM1 | ENSG00000057657 | 0.055432 | 1.9550529 | 9.250668 | 7.295615 | 6 | q21 | PR domain containing 1, with ZNF domain [Source: HGNC Symbol; Acc: 9346] |
| 228624_at | TMEM144 | ENSG00000164124 | 0.055512 | 0.8881157 | 6.820513 | 5.932397 | 4 | q32.1 | transmembrane protein 144 [Source: HGNC Symbol; Acc: 25633] |
| 225626_at | PAG1 | ENSG00000076641 | 0.055625 | 1.6483207 | 9.726492 | 8.078172 | 8 | q21.13 | phosphoprotein associated with glycosphingolipid microdomains 1 [Source: HGNC |
| | | | | | | | | | Symbol; Acc: 30043] |
| 225885_at | EEA1 | ENSG00000102189 | 0.055625 | 0.7278215 | 7.739963 | 7.012141 | 12 | q22 | early endosome antigen 1 [Source: HGNC Symbol; Acc: 3135] |
| 236172_at | LTB4R | ENSG00000213903 | 0.055654 | 1.0840457 | 7.387481 | 6.303435 | 14 | q12 | leukotriene B4 receptor [Source: HGNC Symbol; Acc: 6713] |
| 201953_at | CIB1 | ENSG00000185043 | 0.055677 | 0.7092542 | 9.414667 | 8.705413 | 15 | q26.1 | calcium and integrin binding 1 (calmyrin) [Source: HGNC Symbol; Acc: 16920] |
| 213069_at | HEG1 | ENSG00000173706 | 0.055677 | 1.0582971 | 9.011754 | 7.953457 | 3 | q21.2 | heart development protein with EGF-like domains 1 [Source: HGNC |
| | | | | | | | | | Symbol; Acc: 29227] |
| 226885_at | RNF217 | ENSG00000146373 | 0.055688 | 1.5079517 | 5.917911 | 4.409959 | 6 | q22.31 | ring finger protein 217 [Source: HGNC Symbol; Acc: 21487] |
| 203038_at | PTPRK | ENSG00000152894 | 0.055688 | 1.5495332 | 8.029703 | 6.48017 | 6 | q22.33 | protein tyrosine phosphatase, receptor type, K [Source: HGNC Symbol; Acc: 9674] |
| 240703_s_at | HERC1 | ENSG00000103657 | 0.0557 | 0.9617872 | 6.445278 | 5.483491 | 15 | q22.31 | HECT and RED domain containing E3 ubiquitin protein ligase family member 1 |
| | | | | | | | | | [Source: HGNC Symbol; Acc: 4867] |
| 221641_s_at | ACOT9 | ENSG00000123130 | 0.055792 | 0.5924097 | 9.037041 | 8.444631 | X | p22.11 | acyl-CoA thioesterase 9 [Source: HGNC Symbol; Acc: 17152] |
| 210845_s_at | PLAUR | ENSG00000011422 | 0.055813 | 0.9963797 | 8.866071 | 7.869692 | 19 | q13.31 | plasminogen activator urokinase receptor [Source: HGNC Symbol; Acc: 9053] |
| 212097_at | CAV1 | ENSG00000105974 | 0.055813 | 1.7271465 | 8.888796 | 7.16165 | 7 | q31.2 | caveolin 1, caveolae protein, 22 kDa [Source: HGNC Symbol; Acc: 1527] |
| 243198_at | TEX9 | ENSG00000151575 | 0.055813 | 1.5584665 | 6.115528 | 4.557062 | 15 | q21.3 | testis expressed 9 [Source: HGNC Symbol; Acc: 29585] |
| 204122_at | TYROBP | ENSG00000011600 | 0.055813 | 1.1533455 | 11.48996 | 10.33661 | 19 | q13.12 | TYRO protein tyrosine kinase binding protein [Source: HGNC Symbol; Acc: 12449] |
| 226343_at | DPP8 | ENSG00000074603 | 0.055813 | 0.6675677 | 8.292782 | 7.625214 | 15 | q22.31 | dipeptidyl-peptidase 8 [Source: HGNC Symbol; Acc: 16490] |
| 205715_at | BST1 | ENSG00000109743 | 0.056096 | 0.3449558 | 7.186266 | 6.84131 | 4 | p15.32 | bone marrow stromal cell antigen 1 [Source: HGNC Symbol; Acc: 1118] |
| 211980_at | COL4A1 | ENSG00000187498 | 0.056096 | 1.2236466 | 10.90018 | 9.676529 | 13 | q34 | collagen, type IV, alpha 1 [Source: HGNC Symbol; Acc: 2202] |
| 213572_s_at | SERPINB1 | ENSG00000021355 | 0.056096 | 0.8525835 | 9.44718 | 8.594597 | 6 | p25.2 | serpin peptidase inhibitor, clade B (ovalbumin), member 1 [Source: HGNC |
| | | | | | | | | | Symbol; Acc: 3311] |
| 209047_at | AQP1 | ENSG00000240583 | 0.056096 | 1.7116894 | 8.767181 | 7.055491 | 7 | p14.3 | aquaporin 1 (Colton blood group) [Source: HGNC Symbol; Acc: 633] |
| 214077_x_at | MEIS3P1 | ENSG00000179277 | 0.056096 | 0.9245864 | 8.655448 | 7.730862 | 17 | p12 | Meis homeobox 3 pseudogene 1 [Source: HGNC Symbol; Acc: 7002] |
| 202946_s_at | BTBD3 | ENSG00000132640 | 0.056282 | 1.0081645 | 5.025902 | 4.017738 | 20 | p12.2 | BTB (POZ) domain containing 3 [Source: HGNC Symbol; Acc: 15854] |
| 242953_at | ZNF234 | ENSG00000263002 | 0.056316 | 0.8636423 | 6.410969 | 5.547327 | 19 | q13.31 | zinc finger protein 234 [Source: HGNC Symbol; Acc: 13027] |
| 218380_at | LOC728392 | NA | 0.056643 | 0.6598628 | 8.050296 | 7.390433 | NA | NA | NA |
| 215000_s_at | FEZ2 | ENSG00000171055 | 0.056643 | 0.8955219 | 9.749837 | 8.854315 | 2 | p22.2 | fasciculation and elongation protein zeta 2 (zygin II) [Source: HGNC |
| | | | | | | | | | Symbol; Acc: 3660] |
| 229238_at | C17orf97 | ENSG00000187624 | 0.056643 | 0.7936262 | 7.026858 | 6.233231 | 17 | p13.3 | chromosome 17 open reading frame 97 [Source: HGNC Symbol; Acc: 33800] |
| 219700_at | PLXDC1 | ENSG00000161381 | 0.056643 | 1.3037293 | 7.282377 | 5.978648 | 17 | q12 | plexin domain containing 1 [Source: HGNC Symbol; Acc: 20945] |
| 202432_at | PPP3CB | ENSG00000107758 | 0.056855 | 0.5946658 | 8.65429 | 8.059624 | 10 | q22.2 | protein phosphatase 3, catalytic subunit, beta isozyme [Source: HGNC |
| | | | | | | | | | Symbol; Acc: 9315] |
| 1558711_at | FAM13A-AS1 | ENSG00000248019 | 0.056858 | 0.6634859 | 6.443456 | 5.77997 | 4 | q22.1 | FAM13A antisense RNA 1 [Source: HGNC Symbol; Acc: 19370] |
| 202304_at | FNDC3A | ENSG00000102531 | 0.056858 | 0.9582776 | 8.951197 | 7.992919 | 13 | q14.2 | fibronectin type III domain containing 3A [Source: HGNC Symbol; Acc: 20296] |
| 213429_at | BICC1 | ENSG00000122870 | 0.056858 | 1.4554356 | 5.911437 | 4.456001 | 10 | q21.1 | bicaudal C homolog 1 (Drosophila) [Source: HGNC Symbol; Acc: 19351] |
| 203388_at | ARRB2 | ENSG00000141480 | 0.056858 | 0.7900371 | 8.830551 | 8.040514 | 17 | p13.2 | arrestin, beta 2 (Source: HGNC Symbol; Acc: 712] |
| 205140_at | FPGT | ENSG00000254685 | 0.056863 | 0.7461301 | 7.424392 | 6.678262 | 1 | p31.1 | fucose-1-phosphate guanylyltransferase [Source: HGNC Symbol; Acc: 3825] |
| 226594_at | ENTPD5 | ENSG00000187097 | 0.056942 | 0.5571412 | 7.324717 | 6.767575 | 14 | q24.3 | ectonucleoside triphosphate diphosphohydrolase 5 (Source: HGNC |
| | | | | | | | | | Symbol; Acc: 3367] |
| 219506_at | C1orf54 | ENSG00000118292 | 0.056942 | 1.1443229 | 10.50282 | 9.358498 | 1 | q21.2 | chromosome 1 open reading frame 54 [Source: HGNC Symbol; Acc: 26258] |
| 209230_s_at | NUPR1 | ENSG00000176046 | 0.056997 | 1.1081006 | 11.12844 | 10.02034 | 16 | p11.2 | nuclear protein, transcriptional regulator, 1 [Source: HGNC Symbol; Acc: 29990] |
| 205498_at | GHR | ENSG00000112964 | 0.057095 | 1.3991555 | 5.181886 | 3.782731 | 5 | p13.1 | growth hormone receptor [Source: HGNC Symbol; Acc: 4263] |

TABLE 2

| probeset | gene_symbol | esnsembl_id | q. value | logFC | mean_nonRef | mean_Ref |
|---|---|---|---|---|---|---|
| 230325_at | LOC100133985 | NA | 0.00809665 | −0.911640912 | 5.838505821 | 6.750146733 |
| 231463_at | CNTD1 | ENSG00000176563 | 0.008351401 | −0.538973445 | 3.84343028 | 4.382403725 |
| 1558844_at | LOC100506127 | NA | 0.009456042 | −0.617533967 | 6.109066414 | 6.726600381 |
| 1559507_at | LOC100130357 | NA | 0.009950302 | −0.519619931 | 5.450658035 | 5.970277966 |
| 229378_at | STOX1 | ENSG00000165730 | 0.011768387 | −0.593708902 | 3.913537756 | 4.507246657 |
| 207965_at | NEUROG3 | ENSG00000122859 | 0.012330232 | −0.438710752 | 7.869470101 | 8.308180853 |
| 1552349_a_at | PRSS33 | ENSG00000103355 | 0.013773579 | −0.538188858 | 5.578662199 | 6.116851057 |
| 1566134_at | CARHSP1 | ENSG00000153048 | 0.014454348 | −0.5.56432401 | 6.9668382 | 7.523270601 |
| 206602_s_at | HOXD3 | ENSG00000128652 | 0.016154072 | −0.644932774 | 6.906842818 | 7.551775592 |
| 236374_at | CTXN3 | ENSG00000205279 | 0.016166047 | −0.525752287 | 4.906041398 | 5.431793686 |
| 1560963_a_at | LOC100506379 | NA | 0.016166613 | −0.647132124 | 5.446495421 | 6.093627545 |
| 1553837_at | PGAM5 | ENSG00000247077 | 0.016166613 | −0.466015193 | 6.896008593 | 7.362023785 |
| 1554763_at | UBE2DNL | ENSG00000229547 | 0.016166613 | −0.423010772 | 5.56336289 | 5.986373662 |
| 219840_s_at | TCL6 | ENSG00000187621 | 0.016166613 | −1.34245305 | 4.185464373 | 5.527917423 |
| 222896_at | TMEM38A | ENSG00000072954 | 0.016166613 | −0.778177457 | 6.257085542 | 7.035262999 |
| 205688_at | TFAP4 | ENSG00000090447 | 0.016166613 | −0.616680115 | 7.99573016 | 8.612410275 |
| 1557021_s_at | LOC100507250 | NA | 0.016166613 | −0.767424763 | 6.40032636 | 7.167751123 |
| 237880_at | LOC100506457 | NA | 0.016166613 | −0.776375557 | 6.296009341 | 7.072384898 |
| 210450_at | IGHV5-78 | ENSG00000211978 | 0.016166613 | −0.929279909 | 5.444682435 | 6.373962344 |
| 1570336_at | BDH1 | ENSG00000161267 | 0.016166613 | −0.600927396 | 5.439307091 | 6.040234486 |
| 219076_s_at | PXMP2 | ENSG00000176894 | 0.016166613 | −0.603766933 | 7.746523407 | 8.35029034 |
| 230467_at | TMEM52 | ENSG00000178821 | 0.016166613 | −0.547705024 | 5.782176283 | 6.329881307 |
| 207689_at | TBX10 | ENSG00000167800 | 0.016166613 | −0.58354262 | 5.120413125 | 5.703955745 |
| 210165_at | DNASE1 | ENSG00000213918 | 0.0170385 | −0.638225732 | 6.797117147 | 7.435342379 |
| 1558431_at | NHLRC4 | ENSG00000257108 | 0.017271459 | −0.440024521 | 7.110293049 | 7.55031757 |
| 210122_at | PRM2 | ENSG00000122304 | 0.017305066 | −0.594945443 | 6.297604855 | 6.892550298 |
| 1569270_at | LOC100134368 | NA | 0.017653225 | −0.524095679 | 4.998433733 | 5.522529412 |
| 232781_at | LHX4 | ENSG00000121454 | 0.017653225 | −0.471163014 | 6.387893416 | 6.859056431 |
| 206151_x_at | CELA3B | ENSG00000219073 | 0.0179046 | −0.389566615 | 6.698451608 | 7.088018223 |
| 1553780_at | LINC00638 | ENSG00000258701 | 0.0179681 | −0.442959091 | 5.985279128 | 6.428238219 |
| 1564386_at | TXNDC8 | ENSG00000204193 | 0.018383789 | −0.402808114 | 4.569887895 | 4.972696009 |
| 207116_s_at | GAPDHS | ENSG00000105679 | 0.018896313 | −0.439395893 | 6.265814968 | 6.705210861 |
| 1552564_at | NUDT9P1 | NA | 0.018901839 | −0.627097479 | 5.209804097 | 5.836901576 |
| 1552811_at | WFIKKN1 | ENSG00000127578 | 0.018901839 | −0.407943586 | 7.383936771 | 7.791880357 |
| 244543_s_at | BCDIN3D-AS1 | ENSG00000258057 | 0.018901839 | −0.517606933 | 6.104813842 | 6.622420775 |
| 1555124_at | LOC100129726 | NA | 0.018901839 | −0.737667282 | 6.31928006 | 7.056947342 |
| 1553541_at | LMX1A | ENSG00000162761 | 0.018921376 | −0.477745764 | 4.729705201 | 5.207450965 |
| 1553178_a_at | SSTR3 | ENSG00000183473 | 0.019065055 | −0.49695403 | 6.605956381 | 7.10291041 |
| 1558289_at | RFT1 | ENSG00000163933 | 0.019506232 | −0356946278 | 6.437355142 | 6.794301421 |
| 208034_s_at | PROZ | ENSG00000126231 | 0.019879747 | −0.413177596 | 7.086538428 | 7.499716024 |
| 1553444_a_at | C1orf127 | ENSG00000175262 | 0.019879747 | −0.456161699 | 7.310783447 | 7.766945146 |
| 220077_at | CCDC134 | ENSG00000100147 | 0.019879747 | −0.431568198 | 7.194190518 | 7.625758716 |
| 215756_at | LOC730227 | NA | 0.019879747 | −0.466760373 | 5.640296789 | 6.107057162 |
| 242226_at | LOC100288079 | NA | 0.019879747 | −0.466011928 | 4.501905981 | 4.967917909 |
| 207159_x_at | CRTC1 | ENSG00000105662 | 0.019974794 | −0.342848645 | 8.582700549 | 8.925549194 |
| 1564072_at | MYH16 | ENSG00000002079 | 0.020133959 | −0.513849511 | 5.117985644 | 5.631835155 |
| 206112_at | ANKRD7 | ENSG00000106013 | 0.020139089 | −0.584007767 | 3.631160633 | 4.2151684 |
| 244488_at | LSM14B | ENSG00000149657 | 0.020139089 | −0.471204404 | 5.404535566 | 5.87573997 |
| 243022_at | LOC100506631 | NA | 0.020691131 | −0.472548595 | 6.586231468 | 7.058780063 |
| 223630_at | C7orf13 | ENSG00000244291 | 0.021317074 | −0.621418392 | 6.683076994 | 7.304495386 |
| 207366_at | KCNS1 | ENSG00000124134 | 0.021317074 | −0.522556921 | 5.734453296 | 6.257010217 |
| 1569532_a_at | LCN15 | ENSG00000177984 | 0.021317074 | −0.463360876 | 6.730953106 | 7.194313982 |
| 208291_s_at | TH | ENSG00000180176 | 0.02166097 | −0.435400549 | 5.849357981 | 6.284758531 |
| 239119_at | DNAJC3-AS1 | ENSG00000247400 | 0.022611564 | −0.889794242 | 6.485868079 | 7.375662321 |
| 240185_at | LOC100147773 | NA | 0.022611564 | −0.498977477 | 7.370697097 | 7.869674574 |
| 233843_at | ZBTB12 | ENSG00000204366 | 0.022611564 | −0.519742752 | 7.218271617 | 7.738014369 |
| 1555722_at | SCAMPER | NA | 0.022732168 | −0.334308255 | 3.6043183 | 3.938626555 |
| 1553067_a_at | GNRHR2 | ENSG00000211451 | 0.022732168 | −0.649001368 | 7.399753955 | 8.048755323 |
| 238898_at | LOC101060264 | NA | 0.023506064 | −1.193505286 | 5.871655735 | 7.065161021 |
| 224166_at | SLC25A2 | ENSG00000120329 | 0.023531188 | −0.439944142 | 4.676270398 | 5.11621454 |
| 238239_at | WDR27 | ENSG00000184465 | 0.023577408 | −0.865515668 | 5.475650628 | 6.341276295 |
| 1562039_at | GLYATL1 | ENSG00000166840 | 0.023677408 | −0.494191579 | 3.390583969 | 3.884775548 |
| 207649_at | KRT37 | EN5G00000108417 | 0.023800085 | −0.376179216 | 5.023733968 | 5.399918134 |
| 1559272_at | EXOC3L1 | ENSG00000179044 | 0.023800085 | −0.4129202 | 8.165645994 | 8.578566194 |
| 1556822_s_at | ZNF837 | ENSG00000152475 | 0.023800085 | −0.480490017 | 6.346952069 | 6.827442036 |
| 215323_at | EFR38 | ENSG00000084710 | 0.023922328 | −0.703597987 | 6.556203567 | 7.259801553 |
| 1552932_at | NLRP6 | ENSG00000174885 | 0.024111993 | −0.452619884 | 6.55831599 | 7.010935374 |

| probeset | Chromosome | chrom_band | gene_description |
|---|---|---|---|
| 230325_at | NA | NA | NA |
| 231463_at | 17 | q21.31 | cyclin N-terminal domain containing 1 [Source: HGNC |
| | | | Symbol; Acc: 26847] |
| 1558844_at | NA | NA | NA |
| 1559507_at | NA | NA | NA |
| 229378_at | 10 | q21.3 | storkhead box 1 [Source: HGNC Symbol; Acc: 23508] |
| 207965_at | 10 | q22.1 | neurogenin 3 [Source: HGNC Symbol; Acc: 13806] |
| 1552349_a_at | 16 | p13.3 | protease, serine, 33 [Source: HGNC Symbol Acc: 30405] |
| 1566134_at | 16 | p13.2 | calcium regulated heat stable protein 1, 24 kDa [Source: HGNC |
| | | | Symbol; Acc: 17150] |
| 206602_s_at | 2 | q31.1 | homeobox D3 [Source: HGNC Symbol; Acc: 5137] |
| 236374_at | 5 | q23.2 | cortexin 3 [Source: HGNC Symbol; Acc: 31110] |
| 1560963_a_at | NA | NA | NA |
| 1553837_at | 12 | q24.33 | phosphoglycerate mutase family member 5 [Source: HGNC |
| | | | Symbol; Acc: 28763] |
| 1554763_at | X | q21.1 | ubiquitin-conjugating enzyme E2D N-terminal like (pseudogene) |
| | | | [Source: HGNC Symbol; Acc: 28656] |
| 219840_s_at | 14 | q32.13 | T-cell leukemia/lymphoma 6 (non-protein coding) [Source: HGNC |
| | | | Symbol; Acc: 13463] |
| 222896_at | 19 | p13.11 | transmembrane protein 38A [Source: HGNC Symbol; Acc: 28462] |
| 205688_at | 16 | p13.3 | transcription factor AP-4 (activating enhancer binding protein 4) |
| | | | [Source: HGNC Symbol; Acc: 11745] |
| 1557021_s_at | NA | NA | NA |
| 237880_at | NA | NA | NA |
| 210450_at | 14 | q32.33 | immunoglobulin heavy variable 5-78 (pseudogene) [Source: HGNC |
| | | | Symbol; Acc: 5660] |
| 1570336_at | 3 | q29 | 3-hydroxybutyrate dehydrogenase, type 1 [Source: HGNC |
| | | | Symbol; Acc: 1027] |
| 219076_s_at | 12 | q24.33 | peroxisomal membrane protein 2, 22 kDa [Source: HGNC |
| | | | Symbol; Acc: 9716] |
| 230467_at | 1 | p36.33 | transmembrane protein 52 [Source: HGNC Symbol; Acc: 27916] |
| 207689_at | 11 | q13.2 | T-box 10 [Source: HGNC Symbol; Acc: 11593] |
| 210165_at | 16 | p13.3 | deoxyribonuclease I [Source: HGNC Symbol; Acc: 2956] |
| 1558431_at | 16 | p13.3 | NHL repeat containing 4 [Source: HGNC Symbol; Acc: 26700] |
| 210122_at | 16 | p13.13 | protamine 2 [Source: HGNC Symbol; Acc: 9448] |
| 1569270_at | NA | NA | NA |
| 232781_at | 1 | q25.2 | LIM homeobox 4 [Source: HGNC Symbol; Acc: 21734] |
| 206151_x_at | 1 | p36.12 | chymotrypsin-like elastase family, member 36 [Source: HGNC |
| | | | Symbol; Acc: 15945] |
| 1553780_at | 14 | q32.33 | long intergenic non-protein coding RNA 638 [Source: HGNC |
| | | | Symbol; Acc: 28325] |
| 1564386_at | 9 | q31.3 | thioredoxin domain containing 8 (spermatozoa) [Source: HGNC |
| | | | Symbol; Acc: 31454] |
| 207116_s_at | 19 | q13.12 | glyceraldehyde-3-phosphate dehydrogenase, spermatogenic |
| | | | [Source: HGNC Symbol; Acc: 24864] |
| 1552564_at | NA | NA | NA |
| 1552811_at | 16 | p13.3 | WAP, follistatin/kazal, immunoglobulin, kunitz and netrin domain |
| | | | containing 1 [Source: HGNC Symbol; Acc: 30912] |
| 244543_s_at | 12 | q13.12 | BCDIN3D antisense RNA 1 [Source: HGNC Symbol; Acc: 44113] |
| 1555124_at | NA | NA | NA |
| 1553541_at | 1 | q23.3 | LIM homeobox transcription factor 1, alpha [Source: HGNC |
| | | | Symbol; Acc: 6653] |
| 1553178_a_at | 22 | q13.1 | somatostatin receptor 3 [Source: HGNC Symbol; Acc: 11332] |
| 1558289_at | 3 | p21.1 | RFT1 homolog (S. cerevisiae) [Source: HGNC Symbol; Acc: 30220] |
| 208034_s_at | 13 | q34 | protein Z, vitamin K-dependent plasma glycoprotein [Source: HGNC |
| | | | Symbol; Acc: 9460] |
| 1553444_a_at | 1 | p36.22 | chromosome 1 open reading frame 127 [Source: HGNC |
| | | | Symbol; Acc: 26730] |
| 220077_at | 22 | q13.2 | coiled-coil domain containing 134 [Source: HGNC |
| | | | Symbol; Acc: 26185] |
| 215756_at | NA | NA | NA |
| 242226_at | NA | NA | NA |
| 207159_x_at | 19 | p13.11 | CREB regulated transcription coactivator 1 [Source: HGNC |
| | | | Symbol; Acc: 16062] |
| 1564072_at | 7 | q22.1 | myosin, heavy chain 16 pseudogene [Source: HGNC |
| | | | Symbol; Acc: 31038] |
| 206112_at | 7 | q31.31 | ankyrin repeat domain 7 [Source: HGNC Symbol; Acc: 18588] |
| 244488_at | 20 | q13.33 | LSM14B, SCD6 homolog B (S. cerevisiae) [Source: HGNC |
| | | | Symbol; Acc: 15887] |
| 243022_at | NA | NA | NA |
| 223630_at | 7 | q36.3 | chromosome 7 open reading frame 13 [Source: HGNC |
| | | | Symbol; Acc: 17126] |
| 207366_at | 20 | q13.12 | potassium voltage-gated channel, delayed-rectifier, subfamily S, |
| | | | member 1 [Source: HGNC Symbol; Acc: 6300] |
| 1569532_a_at | 9 | q34.3 | lipocalin 15 [Source: HGNC Symbol; Acc: 33777] |
| 208291_s_at | 11 | p15.5 | tyrosine hydroxylase [Source: HGNC Symbol; Acc: 11782] |
| 239119_at | 13 | q32.1 | DNAJC3 antisense RNA 1 (head to head) [Source: HGNC |
| | | | Symbol; Acc: 39308] |
| 240185_at | NA | NA | NA |
| 233843_at | 6 | p21.33 | zinc finger and BTB domain containing 12 [Source: HGNC |
| | | | Symbol; Acc: 19066] |
| 1555722_at | NA | NA | NA |
| 1553067_a_at | 1 | q21.1 | gonadotropin-releasing hormone (type 2) receptor 2 [Source: HGNC |
| | | | Symbol; Acc: 16341] |
| 238898_at | NA | NA | NA |
| 224166_at | 5 | q31.3 | solute carrier family 25 (mitochondrial carrier; ornithine |
| | | | transporter) member 2 [Source: HGNC Symbol; Acc: 22921] |
| 238239_at | 6 | q27 | WD repeat domain 27 [Source: HGNC Symbol; Acc: 21248] |
| 1562039_at | 11 | q12.1 | glycine-N-acyltransferase-like 1 [Source: HGNC Symbol; Acc: 30519] |
| 207649_at | 17 | q21.2 | keratin 37 [Source: HGNC Symbol; Acc: 6455] |
| 1559272_at | 16 | q22.1 | exocyst complex component 3-like 1 [Source: HGNC |
| | | | Symbol; Acc: 27540] |
| 1556822_s_at | 19 | q13.43 | zinc finger protein 837 [Source: HGNC Symbol; Acc: 25164] |
| 215328_at | 2 | p23.3 | EFR3 homolog B (S. cerevisiae) [Source: HGNC Symbol; Acc: 29155] |
| 1552932_at | 11 | p15.5 | NLR family, pyrin domain containing 6 [Source: HGNC |
| | | | Symbol; Acc: 22944] |

TABLE 3

| probeset | gene_symbol | esnsembl_id | q. value | logFC | mean_nonRef | mean_Ref |
|---|---|---|---|---|---|---|
| 228754_at | SLC6A6 | ENSG00000131389 | 0 | 0.975480525 | 8.981006333 | 8.005525809 |
| 238327_at | ODF3B | ENSG00000177989 | 0.002730266 | 1.030411696 | 8.897771439 | 7.867359743 |
| 208683_at | CAPN2 | ENSG00000162909 | 0.002730266 | 2.05913862 | 9.820197872 | 7.761059252 |
| 218648_at | CRTC3 | ENSG00000140577 | 0.002730266 | 1.393257272 | 9.008043592 | 7.614786319 |
| 225146_at | FAM219A | ENSG00000164970 | 0.002730266 | 0.795746694 | 8.091945884 | 7.29619919 |
| 218589_at | LPAR6 | ENSG00000139679 | 0.002730266 | 2.422573482 | 9.17966374 | 6.757090257 |
| 217762_s_at | RAB31 | ENSG00000168461 | 0.002730266 | 1.749576112 | 10.13226893 | 8.382692819 |
| 201506_at | TGFBI | ENSG00000120708 | 0.002730266 | 1.805547727 | 11.48439996 | 9.67885223 |
| 224862_at | GNAQ | ENSG00000156052 | 0.002730266 | 2.025363394 | 6.970609713 | 4.945246318 |
| 201425_at | ALDH2 | ENSG00000111275 | 0.002730266 | 1.929378657 | 10.6016455 | 8.672266847 |
| 1555851_s_at | SEPW1 | ENSG00000178980 | 0.003120304 | 1.533124384 | 10.54941655 | 9.016292166 |
| 205241_at | SCO2 | ENSG00000130489 | 0.003120304 | 0.892011788 | 9.145278369 | 8.25326658 |
| 218552_at | ECHDC2 | ENSG00000121310 | 0.003120304 | 1.335313564 | 9.234133085 | 7.89881952 |
| 55662_at | C10orf76 | ENSG00000120029 | 0.003120304 | 0.721271142 | 8.212153422 | 7.49088228 |
| 204773_at | IL11RA | ENSG00000137070 | 0.004436682 | 0.917390241 | 7.610982979 | 6.693592738 |
| 202600_s_at | NRIP1 | ENSG00000180530 | 0.004436682 | 1.662365975 | 7.56245491 | 5.900088935 |
| 221802_s_at | KIAA1598 | ENSG00000187164 | 0.004818116 | 1.88945627 | 8.343765469 | 6.454309199 |
| 226000_at | CTTNBP2NL | ENSG00000143079 | 0.005157169 | 1.467286291 | 7.674825326 | 6.207539034 |
| 222484_s_at | CXCL14 | ENSG00000145824 | 0.005173135 | 3.461735657 | 11.01983085 | 7.55809519 |
| 201012_at | ANXA1 | ENSG00000135046 | 0.005187505 | 1.868142161 | 10.78463604 | 8.916493883 |
| 218854_at | DSE | ENSG00000111817 | 0.006453355 | 1.632134259 | 9.397392236 | 7.765257977 |
| 214040_s_at | GSN | ENSG00000148180 | 0.006453355 | 1.345118377 | 10.26280296 | 8.917684579 |
| 201302_at | ANXA4 | ENSG00000196975 | 0.006598142 | 1.701755825 | 9.377173828 | 7.675418003 |
| 208923_at | CYFIP1 | ENSG00000068793 | 0.006598142 | 1.418337073 | 9.308029118 | 7.889692045 |
| 224414_s_at | CARD6 | ENSG00000132357 | 0.007207901 | 1.29867797 | 8.497051244 | 7.198373274 |
| 205945_at | IL6R | ENSG00000160712 | 0.00809665 | 1.765992405 | 8.874117498 | 7.108125092 |
| 200765_x_at | CTNNA1 | ENSG00000044115 | 0.00809665 | 1.117941552 | 9.630839898 | 8.512898346 |
| 230325_at | LOC100133985 | NA | 0.00809665 | −0.911640912 | 5.838505821 | 6.750146733 |
| 223228_at | LDOC1L | ENSG00000188636 | 0.00809665 | 1.239345994 | 8.444983654 | 7.20563766 |
| 225842_at | PHLDA1 | ENSG00000139289 | 0.008190797 | 1.472675991 | 8.297710351 | 6.825034361 |
| 201348_at | GPX3 | ENSG00000211445 | 0.008351401 | 2.169917885 | 11.32422224 | 9.154304357 |
| 219885_at | SLFN12 | ENSG00000172123 | 0.008351401 | 1.235153278 | 6.097789382 | 4.862636104 |
| 212830_at | MEGF9 | ENSG00000106780 | 0.008351401 | 1.243175161 | 6.734084575 | 5.490909414 |
| 231463_at | CTND1 | ENSG00000176563 | 0.008351401 | −0.538973445 | 3.84343028 | 4.382403725 |
| 202687_s_at | TNFSF10 | ENSG00000121858 | 0.00842482 | 1.529352742 | 11.10527322 | 9.575920476 |
| 217731_s_at | ITM2B | ENSG00000136156 | 0.009100886 | 1.449335477 | 10.05152822 | 8.602192738 |
| 218694_at | ARMCX1 | ENSG00000126947 | 0.009456042 | 1.499155444 | 7.418731757 | 5.919576313 |
| 1558844_at | LOC100506127 | NA | 0.009456042 | −0.617533967 | 6.109066414 | 6.726600381 |
| 202944_at | NAGA | ENSG00000198951 | 0.009456042 | 0.994644426 | 8.304684413 | 7.610039987 |
| 212522_at | PDE8A | ENSG00000073417 | 0.009456042 | 1.212471343 | 8.751053401 | 7.538582058 |
| 226247_at | PLEKHA1 | ENSG00000107679 | 0.009456042 | 1.762189016 | 8.231589342 | 6.469400327 |
| 243423_at | TNIP1 | ENSG00000145901 | 0.009620936 | 1.039362698 | 6.139027011 | 5.099664313 |
| 229074_at | EHD4 | ENSG00000103966 | 0.009950302 | 1.116796029 | 8.728408949 | 7.61161292 |
| 1559507_at | LOC100130357 | NA | 0.009950302 | −0.519619931 | 5.450658035 | 5.970277966 |
| 222154_s_at | SPATS2L | ENSG00000196141 | 0.009950302 | 1.239839493 | 9.90915525 | 8.569315758 |
| 200710_at | ACADVL | ENSG00000072778 | 0.011037244 | 0.872008393 | 9.456312268 | 8.584303875 |
| 228791_at | LOC100129502 | NA | 0.011037244 | 1.793891949 | 8.411266603 | 6.617374654 |
| 208634_s_at | MACF1 | ENSG00000127603 | 0.011768387 | 1.234625274 | 10.26299281 | 9.028367538 |
| 242487_at | CC2D1B | ENSG00000154222 | 0.011768387 | 0.523814355 | 6.843606374 | 6.319792019 |
| 218559_s_at | MAFB | ENSG00000204103 | 0.011768387 | 1.849122185 | 11.31545077 | 9.466328588 |
| 201360_at | CST3 | ENSG00000101439 | 0.011768387 | 1.353324788 | 11.64386355 | 10.29053876 |
| 206101_at | ECM2 | ENSG00000106823 | 0.011768387 | 1.728608593 | 7.156510825 | 5.427902231 |
| 218004_at | BSDC1 | ENSG00000160058 | 0.011768387 | 0.541692248 | 8.948162166 | 8.406469918 |
| 36129_at | SGSM2 | ENSG00000141258 | 0.011768387 | 0.893849973 | 9.480069571 | 8.586219598 |
| 227889_at | LPCAT2 | ENSG00000087253 | 0.011768387 | 1.982248155 | 7.953706021 | 5.971457866 |
| 229378_at | STOX1 | ENSG00000165730 | 0.011768387 | −0.593708902 | 3.913537756 | 4.507246657 |
| 218043_s_at | AZI2 | ENSG00000163512 | 0.011768387 | 0.863898887 | 6.644284515 | 5.780385627 |
| 218066_at | SLC12A7 | ENSG00000113504 | 0.011768387 | 1.005344336 | 8.912722012 | 7.907377676 |
| 58780_s_at | ARHGEF40 | ENSG00000165801 | 0.011846577 | 1.485982141 | 7.235422011 | 5.74943987 |
| 200660_at | S100A11 | ENSG00000163191 | 0.012330232 | 1.388286368 | 11.61065539 | 10.22236902 |
| 207965_at | NEUROG3 | ENSG00000122859 | 0.012330232 | −0.438710752 | 7.869470101 | 8.308180853 |
| 212907_at | SLC30A1 | ENSG00000170385 | 0.012330232 | 1.171312069 | 9.350466514 | 8.179154445 |
| 208999_at | SEPT8 | ENSG00000164402 | 0.012827915 | 1.11339681 | 8.412742626 | 7.299345816 |
| 208949_s_at | LGALS3 | ENSG00000131981 | 0.012937259 | 1.466370381 | 11.76909335 | 10.30272297 |
| 218311_at | MAP4K3 | ENSG00000011566 | 0.012937259 | 1.467987958 | 7.560719009 | 6.092731052 |
| 203518_at | LYST | ENSG00000143669 | 0.013773579 | 1.781389247 | 8.315307394 | 6.533918147 |
| 1552349_a_at | PRSS33 | ENSG00000103355 | 0.013773579 | −0.538188858 | 5.578662199 | 6.116851057 |
| 1566134_at | CARHSP1 | ENSG00000153048 | 0.014454348 | −0.556432401 | 6.9668382 | 7.523270601 |
| 204137_at | GPR137B | ENSG00000077585 | 0.015679526 | 2.08875061 | 9.969766837 | 7.881016227 |
| 222217_s_at | SLC27A3 | ENSG00000143554 | 0.015679526 | 0.901664373 | 8.452290275 | 7.550625902 |
| 201505_at | LAMB1 | ENSG00000091136 | 0.015689414 | 1.834716085 | 9.325407825 | 7.490691739 |
| 206602_s_at | HOXD3 | ENSG00000128652 | 0.016154072 | −0.644932774 | 6.906842818 | 7.551775592 |
| 217728_at | S100A6 | ENSG00000197956 | 0.016157189 | 0.961802592 | 11.17652114 | 10.21471854 |
| 225483_at | VPS26B | ENSG00000151502 | 0.016160221 | 0.731464309 | 8.008598811 | 7.277134503 |
| 202686_s_at | AXL | ENSG00000167601 | 0.016163173 | 2.164883727 | 9.749176136 | 7.584292409 |
| 236374_at | CTXN3 | ENSG00000205279 | 0.016166047 | −0.525752287 | 4.906041398 | 5.431793686 |
| 227276_at | PLXDC2 | ENSG00000120594 | 0.016166613 | 1.969191816 | 9.857994183 | 7.888742368 |
| 228185_at | ZNF25 | ENSG00000175395 | 0.016166613 | 1.929397523 | 6.510839476 | 5.581441953 |
| 217892_s_at | LIMA1 | ENSG00000050405 | 0.016166613 | 1.859321745 | 9.968366162 | 8.109044417 |
| 202727_s_at | IFNGR1 | ENSG00000027697 | 0.016166613 | 1.203322531 | 9.855227569 | 8.651905038 |
| 1560963_a_at | LOC100506379 | NA | 0.016166613 | −0.647132124 | 5.446495421 | 6.093627545 |
| 1553837_at | PGAM5 | ENSG00000247077 | 0.016166613 | −0.466015193 | 6.896008593 | 7.362023785 |
| 1554763_at | UBE2DNL | ENSG00000229547 | 0.016166613 | −0.423010772 | 5.56336289 | 5.986373662 |
| 212112_s_at | STX12 | ENSG00000117758 | 0.016166613 | 0.81158935 | 8.768046401 | 7.956457051 |
| 203789_s_at | SEMA3C | ENSG00000075223 | 0.016166613 | 1.826351818 | 6.85114482 | 5.024793002 |
| 200677_at | PTTG1IP | ENSG00000183255 | 0.016166613 | 0.944308232 | 10.81552238 | 9.871214144 |
| 222876_s_at | ADAP2 | ENSG00000184060 | 0.016166613 | 1.230284047 | 9.947909385 | 8.717625338 |
| 210145_at | PLA2G4A | ENSG00000116711 | 0.016166613 | 1.018586961 | 5.59768987 | 4.579102909 |
| 208109_s_at | LINC00597 | NA | 0.016166613 | 1.252213786 | 6.056932621 | 4.804718835 |
| 209651_at | TGFB1I1 | ENSG00000140682 | 0.016166613 | 1.639068946 | 8.383831001 | 6.744762056 |
| 212698_s_at | SEPT10 | ENSG00000186522 | 0.016166613 | 2.029386612 | 6.787501727 | 4.758115114 |
| 212526_at | SPG20 | ENSG00000133104 | 0.016166613 | 1.308040982 | 6.974473237 | 5.666432255 |
| 209684_at | RIN2 | ENSG00000132669 | 0.016166613 | 1.948498746 | 9.250259854 | 7.301761108 |
| 223204_at | FAM198B | ENSG00000164125 | 0.016166613 | 1.556704699 | 7.718116717 | 6.161412018 |
| 200673_at | LAPTM4A | ENSG00000068697 | 0.016166613 | 1.052582957 | 10.80127759 | 9.748694636 |
| 219840_s_at | TCL6 | ENSG00000187621 | 0.016166613 | −1.34245305 | 4.185464373 | 5.527917423 |
| 222896_at | TMEM38A | ENSG00000072954 | 0.016166613 | −0.778177457 | 6.257085542 | 7.035262999 |
| 205688_at | TFAP4 | ENSG00000090447 | 0.016166613 | −0.616680115 | 7.99573016 | 8.612410275 |
| 225384_at | DOCK7 | ENSG00000116641 | 0.016166613 | 0.820345036 | 7.335350684 | 6.515005648 |
| 1557021_s_at | LOC100507250 | NA | 0.016166613 | −0.767424763 | 6.40032636 | 7.167151123 |
| 209210_s_at | FERMT2 | ENSG00000073712 | 0.016166613 | 1.600225002 | 8.650583066 | 7.050358064 |
| 201798_s_at | MYOF | ENSG00000138119 | 0.016166613 | 1.933721388 | 9.900889993 | 7.967168605 |
| 225949_at | NRBP2 | ENSG00000185189 | 0.016166613 | 1.197815793 | 8.229622618 | 7.031806824 |
| 208924_at | RNF11 | ENSG00000123091 | 0.016166613 | 1.31265628 | 7.905885024 | 6.593228744 |
| 209004_s_at | FBXL5 | ENSG00000118564 | 0.016166613 | 1.083805753 | 10.00546225 | 8.921656499 |
| 237880_at | LOC100506457 | NA | 0.016166613 | −0.776375557 | 6.296009341 | 7.072384898 |
| 226743_at | SLFN11 | ENSG00000172716 | 0.016166613 | 1.653027622 | 8.752878393 | 7.09985077 |
| 228573_at | ANTXR2 | ENSG00000163297 | 0.016166613 | 1.223119076 | 6.502216851 | 5.279097776 |
| 222065_s_at | FLII | ENSG00000177731 | 0.016166613 | 0.779423611 | 9.687503137 | 8.908079526 |
| 210450_at | IGHV5-78 | ENSG00000211978 | 0.016166613 | −0.929279909 | 5.444682435 | 6.373962344 |
| 212989_at | SGMS1 | ENSG00000198964 | 0.016166613 | 1.180434511 | 5.341958 | 4.161523489 |
| 202228_s_at | NPTN | ENSG00000156642 | 0.016166613 | 1.131346073 | 9.703866415 | 8.572520342 |
| 227761_at | MYO5A | ENSG00000197535 | 0.016166613 | 1.47770878 | 8.28230981 | 6.80460103 |
| 202133_at | WWTR1 | ENSG00000018408 | 0.016166613 | 1.982540678 | 8.759155252 | 6.776614574 |
| 209960_at | HGF | ENSG00000019991 | 0.016166613 | 1.216221253 | 4.738984711 | 3.522763458 |
| 212203_x_at | IFITM3 | ENSG00000142089 | 0.016166613 | 0.999465366 | 12.83133036 | 11.83186499 |
| 212158_at | SDC2 | ENSG00000169439 | 0.016166613 | 1.694879284 | 8.399184386 | 6.704305102 |
| 224797_at | ARRDC3 | ENSG00000113369 | 0.016166613 | 1.280896615 | 7.049919874 | 5.769023259 |
| 203124_s_at | SLC11A2 | ENSG00000110911 | 0.016166613 | 1.314981642 | 8.210636812 | 6.895655171 |
| 1570336_at | BDH1 | ENSG00000161267 | 0.016166613 | −0.600927396 | 5.439307091 | 6.040234486 |
| 1553034_at | SDCCAG8 | ENSG00000054282 | 0.016166613 | 0.647985397 | 7.598443667 | 6.95045827 |
| 219076_s_at | PXMP2 | ENSG00000176894 | 0.016166613 | −0.603766933 | 7.746523407 | 8.35029034 |
| 230467_at | TMEM52 | ENSG00000178821 | 0.016166613 | −0.547705024 | 5.782176283 | 6.329881307 |
| 226111_s_at | ZNF385A | ENSG00000161642 | 0.016166613 | 1.061296407 | 9.529821659 | 8.468525252 |
| 231579_s_at | TIMP2 | ENSG00000035862 | 0.016166613 | 1.950902389 | 11.29034402 | 9.339441627 |
| 202007_at | NID1 | ENSG00000116962 | 0.016166613 | 1.61713841 | 7.967907666 | 6.350769257 |
| 207689_at | TBX10 | ENSG00000167800 | 0.016166613 | −0.58354262 | 5.120413125 | 5.703955745 |
| 201681_s_at | DLG5 | ENSG00000151208 | 0.016381594 | 1.584354968 | 7.6803766 | 6.096021632 |
| 212761_at | TCF7L2 | ENSG00000148737 | 0.016677902 | 1.565585438 | 7.595305509 | 6.029720072 |
| 224983_at | SCARB2 | ENSG00000138760 | 0.016923479 | 1.31926409 | 9.808332875 | 8.489068785 |
| 218706_s_at | GRAMD3 | ENSG00000155324 | 0.016923479 | 1.12899438 | 6.584780567 | 5.455786187 |
| 210165_at | DNASE1 | ENSG00000213918 | 0.0170385 | −0.638225732 | 6.797117147 | 7.435342879 |
| 203595_s_at | IFIT5 | ENSG00000152778 | 0.0170385 | 1.314122515 | 8.100950556 | 6.786828041 |
| 1558431_at | NHLRC4 | ENSG00000257108 | 0.017271459 | −0.440024521 | 7.110293049 | 7.55031757 |
| 225133_at | KLF3 | ENSG00000109787 | 0.017271459 | 1.877159888 | 7.744132654 | 5.866972766 |
| 210122_at | PRM2 | ENSG00000122304 | 0.017305066 | −0.594945443 | 6.297604855 | 6.892550298 |
| 204396_s_at | GRK5 | ENSG00000198873 | 0.017457757 | 0.977767737 | 8.13471677 | 7.156949033 |
| 1569270_at | LOC100134368 | NA | 0.017653225 | −0.524095679 | 4.998433733 | 5.522529412 |
| 210105_s_at | FYN | ENSG00000010810 | 0.017653225 | 1.615658944 | 10.56933985 | 8.953680911 |
| 201218_at | CTBP2 | ENSG00000175029 | 0.017653225 | 1.464241977 | 7.445365995 | 5.981124019 |
| 203973_s_at | CEBPD | ENSG00000221869 | 0.017653225 | 1.614196567 | 10.46812679 | 8.853930222 |
| 243038_at | RBM43 | ENSG00000184898 | 0.017653225 | 0.968731593 | 7.015389524 | 6.046657931 |
| 212667_at | SPARC | ENSG00000113140 | 0.017653225 | 2.038777809 | 9.760157874 | 7.721380065 |
| 209341_s_at | IKBKB | ENSG00000104365 | 0.017653225 | 0.980949734 | 8.789651207 | 7.808701473 |
| 232781_at | LHX4 | ENSG00000121454 | 0.017653225 | −0.471163014 | 6.387863416 | 6.859056431 |
| 202336_s_at | PAM | ENSG00000145730 | 0.017653225 | 1.318515399 | 9.289020714 | 7.970505314 |
| 216151_x_at | CELA3B | ENSG00000219073 | 0.0179046 | −0.389566615 | 6.698451608 | 7.088018223 |
| 1553780_at | LINC00638 | ENSG00000258701 | 0.0179681 | −0.442959091 | 5.985279128 | 6.428238219 |
| 212298_at | NRP1 | ENSG00000099250 | 0.018067396 | 1.779345664 | 9.257538424 | 7.47819276 |
| 1564386_at | TXNDC8 | ENSG00000204193 | 0.018383789 | −0.402808114 | 4.569887895 | 4.972696009 |
| 221773_at | ELK3 | ENSG00000111145 | 0.018896313 | 1.62071839 | 8.175421184 | 6.554702794 |
| 207116_s_at | GAPDHS | ENSG00000105679 | 0.018896313 | −0.439395893 | 6.265814968 | 6.705210861 |
| 208816_x_at | ANXA2P2 | ENSG00000231991 | 0.018901839 | 0.878549333 | 10.01165886 | 9.133109525 |
| 225188_at | RAPH1 | ENSG00000173166 | 0.018901839 | 2.005188858 | 7.673175419 | 5.667986561 |
| 1552564_at | NUDT9P1 | NA | 0.018901839 | −0.627097479 | 5.209804097 | 5.836901576 |
| 212779_at | KIAA1109 | ENSG00000138688 | 0.018901839 | 0.990071878 | 8.187158776 | 7.197086899 |
| 221748_s_at | TNS1 | ENSG00000079308 | 0.018901839 | 2.020350883 | 8.869430402 | 6.84907952 |
| 1552811_at | WFIKKN1 | ENSG00000127578 | 0.018901839 | −0.407943586 | 7.383936771 | 7.791880357 |
| 244543_s_at | BCDIN3D-AS1 | ENSG00000258057 | 0.018901839 | −0.517606933 | 6.104813842 | 6.622420775 |
| 1555124_at | LOC100129726 | NA | 0.018901839 | −0.737667282 | 6.31928006 | 7.056947342 |
| 213379_at | COQ2 | ENSG00000173085 | 0.018901839 | 0.52373226 | 8.028910727 | 7.505178467 |
| 211684_s_at | DYNC1I2 | ENSG00000077380 | 0.018901839 | 0.822903615 | 8.910623393 | 8.087719778 |
| 201739_at | SGK1 | ENSG00000118515 | 0.018901839 | 1.479761374 | 10.75364976 | 9.273888386 |
| 209090_s_at | SH3GLB1 | ENSG00000097033 | 0.018901839 | 0.961400759 | 9.96248528 | 9.001084521 |
| 225171_at | ARHGAP18 | ENSG00000146376 | 0.018901839 | 1.537074864 | 8.677922388 | 7.140847525 |
| 204517_at | PPIC | ENSG00000168938 | 0.018901839 | 1.458339897 | 7.874058821 | 6.415718925 |
| 213923_at | RAP2B | ENSG00000181467 | 0.018901839 | 1.24137501 | 10.18514033 | 8.943765324 |
| 201590_x_at | ANXA2 | ENSG00000182718 | 0.018901839 | 1.222362695 | 12.76741798 | 11.54505528 |
| 202202_s_at | LAMA4 | ENSG00000112769 | 0.018901839 | 1.095301757 | 9.350315162 | 8.255013405 |
| 218718_at | PDGFC | ENSG00000145431 | 0.018921376 | 2.106384599 | 7.63389478 | 5.527510181 |
| 1553541_at | LMX1A | ENSG00000162761 | 0.018921376 | −0.477745764 | 4.729705201 | 5.207450965 |
| 2061414_s_at | ASAP2 | ENSG00000151693 | 0.018921376 | 1.474771694 | 6.713855258 | 5.239083564 |
| 1553178_a_at | SSTR3 | ENSG00000183473 | 0.019065055 | −0.49695403 | 6.605956381 | 7.10291041 |
| 212169_at | FKBP9 | ENSG00000122642 | 0.019065055 | 1.110752419 | 8.896737181 | 7.785984761 |
| 209348_s_at | MAF | ENSG00000178573 | 0.019065055 | 2.155786041 | 8.892163605 | 6.736377564 |
| 202011_at | TJP1 | ENSG00000104067 | 0.019266989 | 2.093652215 | 8.408239306 | 6.314587092 |
| 226823_at | PHACTR4 | ENSG00000204138 | 0.019506232 | 0.788498176 | 7.61381437 | 6.825316194 |
| 201375_s_at | PPP2CB | ENSG00000104695 | 0.019506232 | 0.883554817 | 10.00679214 | 9.123237324 |
| 1558289_at | RFT1 | ENSG00000163933 | 0.019506232 | −0.356946278 | 6.437355142 | 6.794301421 |
| 202808_at | WBP1L | ENSG00000166272 | 0.019506232 | 0.667279191 | 9.547031071 | 8.879751881 |
| 227911_at | ARHGAP28 | ENSG00000088756 | 0.019879747 | 1.632136074 | 5.975403546 | 4.343267472 |
| 208034_s_at | PROZ | ENSG00000126231 | 0.019879747 | −0.413177596 | 7.086538428 | 7.499716024 |
| 1553444_a_at | C1orf127 | ENSG00000175262 | 0.019879747 | −0.456161699 | 7.310783447 | 7.766945146 |
| 220077_at | CCDC134 | ENSG00000100147 | 0.019879747 | −0.431568198 | 7.194190518 | 7.624758716 |
| 215756_at | LOC730227 | NA | 0.019879747 | −0.466760373 | 5.640296789 | 6.107057162 |
| 206049_at | SELP | ENSG00000174175 | 0.019879747 | 1.710090268 | 7.227626982 | 5.517536714 |
| 202357_s_at | CFB | ENSG00000243649 | 0.019879747 | 1.157364569 | 8.626327441 | 7.468962872 |
| 217497_at | TYMP | ENSG00000025708 | 0.019879747 | 0.851614407 | 8.694768908 | 7.843154501 |
| 204034_at | ETHE1 | ENSG00000105755 | 0.019879747 | 0.528926484 | 8.283720657 | 7.754794174 |
| 210139_s_at | PMP22 | ENSG00000109099 | 0.019879747 | 1.749405657 | 9.219437323 | 7.470031666 |
| 242226_at | LOC100288079 | NA | 0.019879747 | −0.466011928 | 4.501905981 | 4.967917909 |
| 204114_at | NID2 | ENSG00000087303 | 0.019879747 | 1.635433904 | 7.748453279 | 6.113019374 |
| 207159_x_at | CRTC1 | ENSG00000105662 | 0.019974794 | −0.342848645 | 8.582700549 | 8.925549194 |
| 1564072_at | MYH16 | ENSG00000002079 | 0.020133959 | −0.513849511 | 5.117985644 | 5.631835155 |
| 200878_at | EPAS1 | ENSG00000116016 | 0.020133959 | 1.664350584 | 10.74528693 | 9.080936347 |
| 206112_at | ANKRD7 | ENSG00000106013 | 0.020139089 | −0.584007767 | 3.631160633 | 4.2151684 |
| 202446_s_at | PLSCR1 | ENSG00000188313 | 0.020139089 | 1.410605009 | 9.747770469 | 8.33716546 |
| 221139_s_at | CSAD | ENSG00000139631 | 0.020139089 | 1.103365222 | 6.326659677 | 5.223294456 |
| 244488_at | LSM14B | ENSG00000149657 | 0.020139089 | −0.471204404 | 5.404535566 | 5.87573997 |
| 221012_s_at | TRIM8 | ENSG00000171206 | 0.020139089 | 0.902154468 | 9.733515113 | 8.831360645 |
| 1555832_s_at | KLF6 | ENSG00000067082 | 0.020139089 | 1.505090741 | 10.49275643 | 8.987665692 |
| 212859_x_at | MTIE | ENSG00000139089 | 0.020139089 | 1.31163782 | 11.29221774 | 9.98057992 |
| 203729_at | EMP3 | ENSG00000142227 | 0.020691131 | 1.242611864 | 10.80078079 | 9.558168921 |
| 243022_at | LOC100506631 | NA | 0.020691131 | −0.472548595 | 6.586231468 | 7.058780063 |
| 223630_at | C7orf13 | ENSG00000244291 | 0.021317074 | −0.621418392 | 6.683076994 | 7.304495386 |
| 209238_at | STX3 | ENSG00000166900 | 0.021317074 | 1.047521762 | 7.816754018 | 6.769232256 |
| 207366_at | KCNS1 | ENSG00000124134 | 0.021317074 | −0.522556921 | 5.734453296 | 6.257010217 |
| 1569532_a_at | LCN15 | ENSG00000177984 | 0.021317074 | −0.463360876 | 6.730953106 | 7.194313982 |
| 228617_at | XAF1 | ENSG00000132530 | 0.021319587 | 1.859163345 | 9.784426104 | 7.925262758 |
| 204436_at | PLEKHO2 | ENSG00000241839 | 0.02166097 | 0.814909278 | 9.412794546 | 8.597885267 |
| 208291_s_at | TH | ENSG00000180176 | 0.02166097 | −0.435400549 | 5.849357981 | 6.284758531 |
| 1555756_a_at | CLEC7A | ENSG00000172243 | 0.02166097 | 1.948701695 | 9.541407089 | 7.592705395 |
| 202381_at | ADAM9 | ENSG00000168615 | 0.022252306 | 1.522568908 | 9.387992162 | 7.865423254 |
| 202291_s_at | MGP | ENSG00000111341 | 0.022611564 | 2.128960252 | 11.06083826 | 8.931878011 |
| 239119_at | DNAJC3-AS1 | ENSG00000247400 | 0.022611564 | −0.889794242 | 6.485868079 | 7.375662321 |
| 213056_at | FRMD4B | ENSG00000114541 | 0.022611564 | 1.741374115 | 6.023278408 | 4.281904293 |
| 218162_at | OLFML3 | ENSG00000116774 | 0.022611564 | 1.550845738 | 8.328430141 | 6.777584404 |
| 212845_at | SAMD4A | ENSG00000020577 | 0.022611564 | 1.246715477 | 6.634446869 | 5.387731392 |
| 240185_at | LOC100147773 | NA | 0.022611564 | −0.498977477 | 7.370697097 | 7.869674574 |
| 233843_at | ZBTB12 | ENSG00000204366 | 0.022611564 | −0.519742752 | 7.218271617 | 7.738014369 |
| 218541_s_at | C8orf4 | ENSG00000176907 | 0.022732168 | 1.976295335 | 6.69374434 | 4.717449005 |
| 1555722_at | SCAMPER | NA | 0.022732168 | −0.334308255 | 3.6043183 | 3.938626555 |
| 1559005_s_at | CNTLN | ENSG00000044459 | 0.022732168 | 1.390816874 | 6.512894646 | 5.122077773 |
| 218909_at | RPS6KC1 | ENSG00000136643 | 0.022732168 | 0.752157812 | 8.355673576 | 7.603515764 |
| 1553067_a_at | GNRHR2 | ENSG00000211451 | 0.022732168 | −0.649001368 | 7.399753955 | 8.048755323 |
| 226384_at | PPAPDC1B | ENSG00000147535 | 0.022732168 | 1.211118387 | 8.15972622 | 6.948607833 |
| 212509_s_at | MXRA7 | ENSG00000182534 | 0.022732168 | 1.467475866 | 9.764658055 | 8.297182189 |
| 225975_at | PCDH18 | ENSG00000189184 | 0.023159359 | 1.779023771 | 7.236967211 | 5.45794344 |
| 201341_at | ENC1 | ENSG00000171617 | 0.023415903 | 1.444333682 | 8.066608054 | 6.622274373 |
| 203324_s_at | CAV2 | ENSG00000105971 | 0.023506064 | 1.952200967 | 8.152023753 | 6.199822786 |
| 238898_at | LOC101060264 | NA | 0.023506064 | −1.193505286 | 5.871655735 | 7.065161021 |
| 212230_at | PPAP2B | ENSG00000162407 | 0.023506064 | 1.816510197 | 9.071697681 | 7.255187484 |
| 218632_at | HECTD3 | ENSG00000126107 | 0.023506064 | 0.736900447 | 9.627647883 | 8.890747437 |
| 212204_at | TMEM87A | ENSG00000103978 | 0.023515139 | 0.898492789 | 9.007547211 | 8.109054422 |
| 226022_at | SASH1 | ENSG00000111961 | 0.023515139 | 2.002711158 | 7.610795761 | 5.608084603 |
| 224166_at | SLC25A2 | ENSG00000120329 | 0.023531188 | −0.439944142 | 4.676270398 | 5.11621454 |
| 242800_at | NHS | ENSG00000188158 | 0.023677408 | 1.162711533 | 6.713331023 | 5.550619491 |
| 203477_at | COL1SA1 | ENSG00000204291 | 0.023677408 | 1.614306704 | 8.436883195 | 6.822576491 |
| 200857_s_at | NCOR1 | ENSG00000141027 | 0.023677408 | 0.709194263 | 8.662953014 | 7.95375875 |
| 238239_at | WDR27 | ENSG00000184465 | 0.023677408 | −0.865615668 | 5.475660628 | 6.341276295 |
| 1562089_at | GLYATL1 | ENSG00000166840 | 0.023677408 | −0.494191579 | 3.390583969 | 3.884775548 |
| 224996_at | ASPH | ENSG00000198363 | 0.023699601 | 1.523017401 | 7.874015884 | 6.350998483 |
| 223562_at | PARVG | ENSG00000138964 | 0.023699601 | 1.500914846 | 9.669889441 | 8.168974594 |
| 213353_at | ABCA5 | ENSG00000154265 | 0.023699601 | 0.996243159 | 8.332970994 | 7.336727836 |
| 209593_s_at | TOR1B | ENSG00000136816 | 0.023800085 | 0.46611305 | 7.855147944 | 7.389034893 |
| 207649_at | KRT37 | ENSG00000108417 | 0.023800085 | −0.376179216 | 5.023738968 | 5.399918184 |
| 32626_at | SGSH | ENSG00000181523 | 0.023800085 | 0.781709479 | 9.793809878 | 9.012100399 |
| 1559272_at | EXOC3L1 | ENSG00000179044 | 0.023800085 | −0.4129202 | 8.165645994 | 8.578566194 |
| 234994_at | TMEM200A | ENSG00000164484 | 0.023800085 | 1.612047423 | 6.524547814 | 4.91250039 |
| 1556822_s_at | ZNF837 | ENSG00000152475 | 0.023800085 | −0.480490017 | 6.346952069 | 6.857442086 |
| 211456_x_at | MT1P2 | NA | 0.023800085 | 1.026714169 | 11.3370169 | 10.31030273 |
| 215328_at | EFR3B | ENSG00000084710 | 0.023922328 | −0.703597987 | 6.556203567 | 7.259801553 |
| 203501_at | CPQ | ENSG00000104324 | 0.024111993 | 0.986206289 | 7.824447671 | 6.838241383 |
| 226155_at | FAM160B1 | ENSG00000151553 | 0.024111993 | 0.94991516 | 8.161808151 | 7.21189299 |
| 1552932_at | NLRP6 | ENSG00000174885 | 0.024111993 | −4.52619884 | 6.55831599 | 7.010935874 |
| 1552258_at | LINC00152 | ENSG00000222041 | 0.024124457 | 1.12452576 | 8.139434844 | 7.014909085 |
| 238025_at | MLKL | ENSG00000168404 | 0.02486642 | 1.215990498 | 8.756992858 | 7.54100236 |
| 219460_s_at | TMEM127 | ENSG00000135956 | 0.02486642 | 0.454625359 | 8.62922137 | 8.174596011 |
| 225522_at | AAK1 | ENSG00000115977 | 0.02486642 | 1.172801595 | 8.65290817 | 7.480106575 |
| 217757_at | A2M | ENSG00000175899 | 0.024980362 | 1.576462299 | 12.02894127 | 10.45247897 |
| 223168_at | RHOU | ENSG00000116574 | 0.025426902 | 1.560384918 | 7.215568938 | 5.655184019 |
| 1561615_s_at | SLC8A1 | ENSG00000183023 | 0.025807759 | 1.56503616 | 8.855136737 | 7.290100577 |
| 203758_at | CTSO | ENSG00000256043 | 0.02606631 | 1.156055866 | 9.31162358 | 8.155567714 |
| 226552_at | IER5L | ENSG00000188483 | 0.026467277 | 0.93672703 | 7.611941271 | 6.675214241 |
| 202766_s_at | FBN1 | ENSG00000166147 | 0.027121311 | 1.374537951 | 7.745362428 | 6.370824476 |
| 225629_s_at | ZBTB4 | ENSG00000174282 | 0.027121311 | 1.019746013 | 9.543580585 | 8.523834572 |
| 212136_at | ATP2B4 | ENSG00000058668 | 0.027282655 | 1.526692329 | 8.833219126 | 7.306526797 |
| 1555881_s_at | LZTS2 | ENSG00000107816 | 0.027517178 | 0.440221226 | 7.841075386 | 7.400854161 |
| 57715_at | CALHM2 | ENSG00000138172 | 0.027517178 | 0.696007379 | 8.099815478 | 7.403808099 |
| 226601_at | SLC30A7 | ENSG00000162695 | 0.027517178 | 0.937218503 | 8.338967731 | 7.401749228 |
| 217890_s_at | PARVA | ENSG00000197702 | 0.027517178 | 1.563831784 | 9.16028565 | 7.596453867 |
| 207624_s_at | RPGR | ENSG00000156313 | 0.027571838 | 0.526289635 | 7.293504241 | 6.767214606 |
| 212372_at | MYH10 | ENSG00000133026 | 0.027571838 | 1.055713787 | 8.323251043 | 7.267537256 |
| 226820_at | ZNF362 | ENSG00000160094 | 0.027571838 | 0.806241318 | 8.807479051 | 8.001237733 |
| 202551_s_at | CRIM1 | ENSG00000150938 | 0.027571838 | 1.425695658 | 8.947673951 | 7.521978294 |
| 208030_s_at | ADD1 | ENSG00000087274 | 0.027642759 | 0.584654998 | 9.931156175 | 9.346501177 |
| 206618_at | IL18R1 | ENSG00000115604 | 0.027642759 | 1.481537358 | 7.518533037 | 6.03699568 |
| 201212_at | LGMN | ENSG00000100600 | 0.027659186 | 2.066812025 | 10.1698232 | 8.103011174 |
| 208782_at | FSTL1 | ENSG00000163430 | 0.027736606 | 1.32524447 | 10.08689397 | 8.761649497 |
| 209191_at | TUBB6 | ENSG00000176014 | 0.027736606 | 1.657527449 | 9.832241239 | 8.17471379 |
| 206875_s_at | SLK | ENSG00000065613 | 0.027736606 | 1.064373439 | 8.057717065 | 6.993343627 |
| 212990_at | SYNJ1 | ENSG00000159082 | 0.027736606 | 1.044260381 | 7.114954309 | 6.070693928 |
| 204193_at | CHKB | ENSG00000100288 | 0.027736606 | 0.899813254 | 9.518634166 | 8.618820912 |
| 203243_s_at | PDLIM5 | ENSG00000163110 | 0.028029546 | 1.142844616 | 7.842659274 | 6.699814657 |
| 227554_at | MAGI2-AS3 | ENSG00000234456 | 0.028029546 | 1.583516525 | 7.049678455 | 5.46616193 |
| 224616_at | DYNC1LI2 | ENSG00000135720 | 0.028029546 | 0.795711906 | 10.00717568 | 9.211463776 |
| 218901_at | PLSCR4 | ENSG00000114698 | 0.028186238 | 1.641200848 | 5.938606594 | 4.297405747 |
| 209050_s_at | RALGDS | ENSG00000160271 | 0.028198657 | 0.830143523 | 9.509798483 | 8.67965496 |
| 209472_at | CCBL2 | ENSG00000137944 | 0.028198657 | 0.791465989 | 9.348801739 | 8.55733575 |
| 227542_at | SOCS6 | ENSG00000170677 | 0.028198657 | 1.310031965 | 6.680388823 | 5.370356858 |
| 204039_at | CEBPA | ENSG00000245848 | 0.028198657 | 0.929871866 | 8.572792639 | 7.642920773 |
| 212586_at | CAST | ENSG00000153113 | 0.028198657 | 0.741294527 | 9.204773928 | 8.463479401 |
| 201280_s_at | DAB2 | ENSG00000153071 | 0.028198657 | 1.77978079 | 8.603166646 | 6.823385856 |
| 201599_at | OAT | ENSG00000065154 | 0.028198657 | 0.889981447 | 9.474237119 | 8.584255672 |
| 225334_at | C10orf32 | ENSG00000166275 | 0.028198657 | 0.781438768 | 8.253485336 | 7.472046568 |
| 201334_s_at | ARHGEF12 | ENSG00000196914 | 0.028226747 | 1.120752315 | 9.211231463 | 8.090479148 |
| 229041_s_at | ITGB2-AS1 | ENSG00000227039 | 0.028919999 | 1.834305085 | 9.515397779 | 7.681092694 |
| 212124_at | ZMIZ1 | ENSG00000108175 | 0.028919999 | 1.472001753 | 10.25152688 | 8.779525132 |
| 204863_s_at | IL6ST | ENSG00000134352 | 0.028919999 | 1.145080811 | 9.161299338 | 8.016218527 |
| 215235_at | SPTAN1 | ENSG00000197694 | 0.028919999 | 0.687712354 | 10.61620713 | 9.928494779 |
| 212606_at | WDFY3 | ENSG00000163625 | 0.028919999 | 1.687587613 | 6.840272287 | 5.152684674 |
| 212601_at | ZZEF1 | ENSG00000074755 | 0.028919999 | 0.462251252 | 8.027308146 | 7.565056894 |
| 228577_x_at | ODF2L | ENSG00000122417 | 0.028919999 | 0.928335466 | 7.134280411 | 6.205944945 |
| 227776_at | ACER3 | ENSG00000078124 | 0.028919999 | 1.179421743 | 7.521437369 | 6.342015626 |
| 201146_at | NFE2L2 | ENSG00000116044 | 0.028919999 | 0.970559163 | 10.2023661 | 9.231806934 |
| 214807_at | LOC100509635 | NA | 0.028919999 | 1.839892443 | 8.320892858 | 6.481000416 |
| 201185_at | HTRA1 | ENSG00000166033 | 0.028919999 | 1.815072013 | 9.750466378 | 7.935394366 |
| 209120_at | NR2F2 | ENSG00000185551 | 0.029112343 | 2.191435636 | 8.372704969 | 6.181269333 |
| 226021_at | RDH10 | ENSG00000121039 | 0.029112343 | 1.181075712 | 6.837596657 | 5.656520945 |
| 224480_s_at | AGPAT9 | ENSG00000138678 | 0.029340633 | 1.110871028 | 4.910358231 | 3.799487203 |
| 212077_at | CALD1 | ENSG00000122786 | 0.029340633 | 1.724790131 | 10.17919504 | 8.454404905 |
| 204745_x_at | MT1G | ENSG00000124144 | 0.029340633 | 1.041896811 | 10.90300528 | 9.861108467 |
| 227930_at | AGO4 | ENSG00000134698 | 0.029585591 | 1.105352072 | 6.925534304 | 5.820182232 |
| 212636_at | QKI | ENSG00000112531 | 0.029710813 | 1.378340651 | 8.509471015 | 7.131130365 |
| 202609_at | EPS8 | ENSG00000151491 | 0.029743118 | 2.016395991 | 8.302002557 | 6.285606566 |
| 207791_s_at | RAB1A | ENSG00000138069 | 0.029761404 | 0.834242654 | 9.926540665 | 9.092298011 |
| 226377_at | NFIC | ENSG00000141905 | 0.029761404 | 1.124935006 | 8.785641229 | 7.660706223 |
| 212607_at | AKT3 | ENSG00000117020 | 0.029761404 | 1.28613632 | 8.86790134 | 7.58176502 |
| 220122_at | MCTP1 | ENSG00000175471 | 0.029807901 | 1.579458936 | 7.121595256 | 5.54213632 |
| 225941_at | EIF4E3 | ENSG00000163412 | 0.029807901 | 1.364241317 | 7.790818489 | 6.426577172 |
| 65635_at | ENGASE | ENSG00000167280 | 0.029836343 | 0.549126535 | 9.240446513 | 8.691319977 |
| 202897_at | SIRPA | ENSG00000198053 | 0.029836343 | 1.079213131 | 9.722842102 | 8.643628972 |
| 213817_at | IRAK3 | ENSG00000090376 | 0.029836343 | 1.432000131 | 6.594781364 | 5.162781233 |
| 204703_at | IFT88 | ENSG00000032742 | 0.029836343 | 0.555626502 | 7.914095211 | 7.358468709 |
| 201029_s_at | CD99 | ENSG00000002586 | 0.029836343 | 1.179550753 | 11.79489029 | 10.61533954 |
| 201105_at | LGALS1 | ENSG00000100097 | 0.029836343 | 1.067969519 | 12.55395514 | 11.48598563 |
| 228666_at | C15orf38 | ENSG00000242498 | 0.029836343 | 0.965528767 | 8.632863432 | 7.667334665 |
| 213733_at | MYO1F | ENSG00000142347 | 0.029836343 | 1.202020923 | 9.0479833 | 7.835962377 |
| 225162_at | SH3D19 | ENSG00000109686 | 0.029836343 | 1.697719881 | 6.304495587 | 4.606775706 |
| 221766_s_at | FAM46A | ENSG00000112773 | 0.030025868 | 1.307438215 | 9.102394105 | 7.79495589 |
| 235256_s_at | GALM | ENSG00000143891 | 0.030025868 | 1.3208648 | 7.939207095 | 6.618342295 |
| 201417_at | SOX4 | ENSG00000124766 | 0.030025868 | 1.094353867 | 7.314555393 | 6.220201526 |
| 201963_at | ACSL1 | ENSG00000151726 | 0.030025868 | 1.026257459 | 9.481284578 | 8.455027119 |
| 218983_at | C1RL | ENSG00000139178 | 0.030025868 | 1.053541158 | 6.970689021 | 5.917147863 |
| 225442_at | DDR2 | ENSG00000162733 | 0.030025868 | 1.204158025 | 8.120903012 | 6.916744987 |
| 225128_at | KDELC2 | ENSG00000178202 | 0.030088928 | 0.839868955 | 7.886197408 | 7.046328453 |
| 225913_at | PEAK1 | NA | 0.030088928 | 0.877481347 | 8.926047545 | 8.048566198 |
| 238477_at | KIF1C | ENSG00000129250 | 0.030088928 | 0.514313913 | 6.616651948 | 6.102338035 |
| 1557236_at | APOL6 | ENSG00000221963 | 0.030333182 | 1.353018745 | 6.588334921 | 5.235316176 |
| 224764_at | ARHGAP21 | ENSG00000126775 | 0.030504136 | 0.72625482 | 8.341027574 | 7.614772751 |
| 204568_at | ATG14 | ENSG00000189171 | 0.030504136 | 0.68255677 | 7.833224815 | 7.150668045 |
| 202598_at | S100A13 | ENSG00000171729 | 0.030504136 | 0.905469594 | 10.25862756 | 9.353157962 |
| 218815_s_at | TMEM51 | ENSG00000118508 | 0.030529954 | 0.821120093 | 9.101393989 | 8.280273897 |
| 204214_s_at | RAB32 | ENSG00000165914 | 0.030529954 | 0.974779135 | 8.206092346 | 7.231313211 |
| 226152_at | TTC7B | ENSG00000172575 | 0.030555177 | 1.225826179 | 7.058997136 | 5.833170957 |
| 205590_at | RASGRP1 | ENSG00000173482 | 0.030555177 | 1.811511239 | 10.05801133 | 8.246500094 |
| 1555579_s_at | PTPRM | ENSG00000172059 | 0.030555177 | 1.133321622 | 7.834017042 | 6.70069542 |
| 218486_at | KLF11 | ENSG00000157240 | 0.030555177 | 1.276283669 | 7.737115935 | 6.460832266 |
| 204451_at | FZD1 | ENSG00000172059 | 0.030555177 | 1.280052899 | 7.241574346 | 5.961521446 |
| 226186_at | TMOD2 | ENSG00000157240 | 0.030555177 | 1.362546177 | 6.78422602 | 5.421679843 |
| 225288_at | COL27A1 | ENSG00000196739 | 0.030691053 | 1.40544216 | 7.835001659 | 6.429559499 |
| 225163_at | FRMD4A | ENSG00000151474 | 0.030947129 | 1.137531712 | 7.41162528 | 6.274093568 |
| 221541_at | CRISPLD2 | ENSG00000103196 | 0.030947129 | 1.757392292 | 8.718881123 | 6.961488831 |
| 230480_at | PIWIL4 | ENSG00000134627 | 0.030947129 | 0.657514674 | 6.112214345 | 5.45469967 |
| 227444_at | ARMCX4 | ENSG00000196440 | 0.030947129 | 0.976979963 | 6.028166519 | 5.051186556 |
| 218285_s_at | BDH2 | ENSG00000164039 | 0.030947129 | 0.724767228 | 8.942667027 | 8.217899799 |
| 209687_at | CXCL12 | ENSG00000107562 | 0.030947129 | 2.225058448 | 9.716134003 | 7.491075555 |
| 203261_at | DCTN6 | ENSG00000104671 | 0.030947129 | 0.806565337 | 10.35688135 | 9.550316016 |
| 227001_at | NIPAL2 | ENSG00000104361 | 0.030947129 | 1.385419946 | 8.211606253 | 6.826186308 |
| 213119_at | SLC36A1 | ENSG00000123643 | 0.030947129 | 0.527649011 | 8.188390174 | 7.660741163 |
| 201089_at | ATP6V1B2 | ENSG00000147416 | 0.030947129 | 0.97966903 | 10.05201136 | 9.072342335 |
| 228937_at | LACC1 | ENSG00000179630 | 0.030947129 | 1.434772368 | 7.870868743 | 6.436096375 |
| 219666_at | MS4A6A | ENSG00000110077 | 0.031011469 | 2.158907191 | 10.17588351 | 8.016976319 |
| 209160_at | AKR1C3 | ENSG00000196139 | 0.031011469 | 1.558340801 | 6.702549633 | 5.144208832 |
| 203688_at | PKD2 | ENSG00000118762 | 0.031011469 | 1.22466397 | 8.101780908 | 6.877116938 |
| 209379_s_at | CCSER2 | ENSG00000107771 | 0.031011469 | 0.895302988 | 6.749214848 | 5.85391186 |
| 202973_x_at | FAM13A | ENSG00000138640 | 0.031011469 | 1.509075891 | 6.614676319 | 5.105600428 |
| 222999_s_at | CCNL2 | ENSG00000221978 | 0.031011469 | 0.586307213 | 9.418617885 | 8.832310673 |
| 32811_at | MYO1C | ENSG00000197879 | 0.031087661 | 0.640476944 | 9.336456528 | 8.695979584 |
| 213737_x_at | GOLGA8I | ENSG00000153666 | 0.031114275 | 1.284101363 | 9.220785563 | 7.9366842 |
| 202351_at | ITGAV | ENSG00000138448 | 0.031169046 | 1.360408955 | 9.310596989 | 7.950188034 |
| 226066_at | MITF | ENSG00000187098 | 0.031203036 | 1.394602359 | 8.259922404 | 6.865320045 |
| 212453_at | KIAA1279 | ENSG00000198954 | 0.031374519 | 0.701033594 | 7.66960335 | 6.968569756 |
| 223276_at | SMIM3 | ENSG00000256235 | 0.031482467 | 1.057195825 | 7.313511684 | 6.256315859 |
| 63825_at | ABHD2 | ENSG00000140526 | 0.031482467 | 1.051003553 | 8.787774645 | 7.736771092 |
| 203817_at | GUCY1B3 | ENSG00000061918 | 0.031482467 | 1.374252812 | 7.677665745 | 6.303412934 |
| 232090_at | DNM3OS | ENSG00000230630 | 0.031482467 | 1.876637903 | 6.738189882 | 4.861551979 |
| 218292_s_at | PRKAG2 | ENSG00000106617 | 0.03162987 | 0.737763701 | 7.332144993 | 6.594381292 |
| 1558173_a_at | LUZP1 | ENSG00000169641 | 0.03162987 | 0.796546664 | 9.005566311 | 8.209019647 |
| 201438_at | COL6A3 | ENSG00000163359 | 0.03162987 | 1.673097599 | 11.52000403 | 9.846906433 |
| 235458_at | HAVCR2 | ENSG00000135077 | 0.03162987 | 1.567140584 | 9.031225144 | 7.46408456 |
| 223393_s_at | TSHZ3 | ENSG00000121297 | 0.03162987 | 1.323992867 | 7.607905295 | 6.283912428 |
| 215706_x_at | ZYX | ENSG00000159840 | 0.031657402 | 0.877362553 | 10.7162578 | 9.838895242 |
| 219315_s_at | TMEM204 | ENSG00000131634 | 0.031799565 | 0.945133516 | 7.656936821 | 6.711803305 |
| 201296_s_at | WSB1 | ENSG00000109406 | 0.031799565 | 1.262242726 | 10.19943828 | 8.937195558 |
| 232231_at | RUNX2 | ENSG00000124813 | 0.031799565 | 2.284394978 | 8.680094375 | 6.395699398 |
| 226225_at | MCC | ENSG00000171444 | 0.031799565 | 1.796214442 | 5.490904796 | 3.694690354 |
| 214660_at | ITGA1 | ENSG00000213949 | 0.031799565 | 0.935911859 | 4.662646523 | 3.726734663 |
| 216903_s_at | MICU1 | ENSG00000107745 | 0.031799565 | 0.517945824 | 8.82526293 | 8.307317106 |
| 215111_s_at | TSC22D1 | ENSG00000102804 | 0.031799565 | 1.489649253 | 9.321974172 | 7.832324919 |
| 200782_at | ANXA5 | ENSG00000164111 | 0.031799565 | 0.961354796 | 10.84305118 | 9.881696385 |
| 226771_at | ATP8B2 | ENSG00000143515 | 0.031799565 | 1.091181637 | 8.208903918 | 7.117722281 |
| 212185_x_at | MT2A | ENSG00000124148 | 0.031799565 | 1.014640934 | 12.3668934 | 11.35225247 |
| 200986_at | SERPING1 | ENSG00000149131 | 0.031970706 | 1.239835886 | 10.45704492 | 9.217209036 |
| 221269_s_at | SH3BGRL3 | ENSG00000142669 | 0.031970706 | 0.564410347 | 11.47106325 | 10.9066529 |
| 202081_at | IER2 | ENSG00000160888 | 0.031970706 | 1.223949737 | 10.27326737 | 9.049317631 |
| 206995_x_at | SCARF1 | ENSG00000074660 | 0.0324388 | 0.731394685 | 6.493193087 | 5.761798402 |
| 204963_at | SSPN | ENSG00000123096 | 0.032493399 | 1.977402115 | 7.971609463 | 5.994207348 |
| 202192_s_at | GAS7 | ENSG00000007237 | 0.032688533 | 1.055406528 | 9.145390855 | 8.089984326 |
| 224747_at | UBE2Q2 | ENSG00000140367 | 0.032688533 | 1.004434491 | 8.252426793 | 7.247992302 |
| 202136_at | ZMYND11 | ENSG00000015171 | 0.032688533 | 1.001702173 | 9.778629696 | 8.776927523 |
| 209970_x_at | CASP1 | ENSG00000137752 | 0.032709967 | 1.260485478 | 9.149474702 | 7.888989224 |
| 234987_at | SAMHD1 | ENSG00000101347 | 0.032726724 | 1.416504141 | 9.200321126 | 7.783816985 |
| 205173_x_at | CD58 | ENSG00000116815 | 0.032726724 | 1.604544482 | 9.908500357 | 8.303955875 |
| 229732_at | ZNF823 | ENSG00000197933 | 0.032726724 | 0.521251726 | 4.981580656 | 4.46032893 |
| 37408_at | MRC2 | ENSG00000011028 | 0.032726724 | 0.976917275 | 9.119459522 | 8.142542247 |
| 208922_s_at | NXF1 | ENSG00000162231 | 0.032726724 | 0.716558245 | 10.10896656 | 9.392408317 |
| 200766_at | CTSD | ENSG00000117984 | 0.032726724 | 1.031649441 | 11.05559476 | 10.02394532 |
| 209129_at | TRIP6 | ENSG00000087077 | 0.032726724 | 0.614395443 | 8.270124739 | 7.655729295 |
| 228728_at | CPED1 | ENSG00000106034 | 0.032726724 | 1.325706695 | 6.216332514 | 4.890625819 |
| 223028_s_at | SNX9 | ENSG00000130340 | 0.032726724 | 1.546959073 | 9.600897425 | 8.053938352 |
| 214464_at | CDC42BPA | ENSG00000143776 | 0.032726724 | 1.59035691 | 8.71190947 | 7.12155256 |
| 204059_s_at | ME1 | ENSG00000065833 | 0.032726724 | 2.130953781 | 7.883910609 | 5.752956829 |
| 202481_at | DHRS3 | ENSG00000162496 | 0.032726724 | 1.355717458 | 9.373900183 | 8.018182725 |
| 201426_s_at | VIM | ENSG00000026025 | 0.032726724 | 0.861969983 | 13.16643703 | 12.30446705 |
| 214177_s_at | PBXIP1 | ENSG00000163346 | 0.032726724 | 0.643944864 | 8.558973921 | 7.915029058 |
| 218793_s_at | SCML1 | ENSG00000047634 | 0.032726724 | 1.794930413 | 7.348371208 | 5.553440795 |
| 201163_s_at | IGFBP7 | ENSG00000163453 | 0.032726724 | 1.277289099 | 11.02178563 | 9.744496534 |
| 225406_at | TWSG1 | ENSG00000128791 | 0.032726724 | 1.015495797 | 8.499218476 | 7.486722679 |
| 209071_s_at | RGS5 | ENSG00000143248 | 0.032726724 | 1.999256143 | 9.334305908 | 7.335049765 |
| 204206_at | MNT | ENSG00000070444 | 0.032726724 | 0.454316598 | 8.034279619 | 7.579963021 |
| 226026_at | DIRC2 | ENSG00000138463 | 0.032726724 | 1.212593407 | 8.349561256 | 7.136967849 |
| 214683_s_at | CLK1 | ENSG00000013441 | 0.032726724 | 0.921509328 | 10.05348313 | 9.131973801 |
| 218095_s_at | TMEM165 | ENSG00000134851 | 0.032726724 | 0.788171748 | 10.38714798 | 9.598976234 |
| 219316_s_at | FLVCR2 | ENSG00000119686 | 0.032726724 | 0.85580157 | 7.371184456 | 6.515382886 |
| 226763_at | SESTD1 | ENSG00000187231 | 0.032726724 | 1.059647685 | 9.5497248 | 8.490077115 |
| 227361_at | HS3ST3B1 | ENSG00000125430 | 0.032726724 | 1.522718422 | 7.752502237 | 6.229783816 |
| 205248_at | DOPEY2 | ENSG00000142197 | 0.032726724 | 0.619808007 | 7.178374889 | 6.558566882 |
| 211026_s_at | MGLL | ENSG00000074416 | 0.032726724 | 1.34626057 | 9.704250583 | 8.357990013 |
| 212224_at | ALDH1A1 | ENSG00000165092 | 0.032726724 | 2.327986623 | 9.644018097 | 7.316031474 |
| 201215_at | PLS3 | ENSG00000102024 | 0.032726724 | 1.846069002 | 7.903700354 | 6.057631352 |
| 218432_at | FBXO3 | ENSG00000110429 | 0.032726724 | 0.969202811 | 6.759587278 | 5.790384467 |
| 212428_at | KIAA0368 | ENSG00000136813 | 0.032726724 | 0.502712224 | 8.880960464 | 8.37824824 |
| 203394_s_at | HES1 | ENSG00000114315 | 0.032726724 | 1.18052545 | 9.074966195 | 7.894440745 |
| 235125_x_at | FAM73A | ENSG00000180488 | 0.032726724 | 0.78900753 | 6.551390988 | 5.762383458 |
| 224690_at | FAM210B | ENSG00000124098 | 0.032726724 | 1.21859774 | 8.947455594 | 7.728857855 |
| 219991_at | SLC2A9 | ENSG00000109667 | 0.032726724 | 0.745206521 | 7.42728002 | 6.6820735 |
| 214560_at | FPR3 | ENSG00000187474 | 0.032726724 | 1.552547402 | 10.11158701 | 8.559039603 |
| 210946_at | PPAP2A | ENSG00000067113 | 0.032726724 | 0.869426384 | 8.48212043 | 7.612694046 |
| 210817_s_at | CALCOCO2 | ENSG00000136436 | 0.032726724 | 0.901072122 | 10.04861929 | 9.147547173 |
| 208636_at | ACTN1 | ENSG00000072110 | 0.032726724 | 1.492130835 | 9.059966788 | 7.567835952 |
| 1561226_at | XCR1 | ENSG00000173578 | 0.032726724 | 0.928038272 | 4.98061814 | 4.052579867 |
| 229256_at | PGM2L1 | ENSG00000165434 | 0.032726724 | 0.950253856 | 6.806744083 | 5.856490226 |
| 228131_at | ERCC1 | ENSG00000012061 | 0.032726724 | 0.698680629 | 8.644377717 | 7.945697089 |
| 209310_s_at | CASP4 | ENSG00000196954 | 0.032735931 | 0.910389382 | 10.59028021 | 9.679890826 |
| 218729_at | LXN | ENSG00000079257 | 0.032735931 | 1.373435682 | 9.36297787 | 7.989542188 |
| 227274_at | SYNJ2BP | ENSG00000213463 | 0.032735931 | 0.945655293 | 8.969654087 | 8.023998795 |
| 219147_s_at | NMRK1 | ENSG00000106733 | 0.032835037 | 1.031303038 | 8.134786202 | 7.103483164 |
| 205083_at | AOX1 | ENSG00000138356 | 0.032835037 | 1.344757535 | 6.346468615 | 5.00171108 |
| 209109_s_at | TSPAN6 | ENSG00000000003 | 0.032869735 | 1.316302616 | 7.197532473 | 5.881229857 |
| 225303_at | KIRREL | ENSG00000183853 | 0.032869735 | 1.284466775 | 7.658036153 | 6.373569378 |
| 225185_at | MRAS | ENSG00000158186 | 0.032869735 | 1.037700745 | 7.834458987 | 6.796758242 |
| 203716_s_at | DPP4 | ENSG00000197635 | 0.032869735 | 1.50992049 | 8.788771757 | 7.278851267 |
| 204342_at | SLC25A24 | ENSG00000085491 | 0.032869735 | 0.999800872 | 8.623812513 | 7.624011641 |
| 202948_at | IL1R1 | ENSG00000115594 | 0.033187995 | 1.749785268 | 8.584052713 | 6.834267445 |
| 212511_at | PICALM | ENSG00000073921 | 0.033187995 | 0.742253285 | 6.915631282 | 6.173377997 |
| 214429_at | MTMR6 | ENSG00000139505 | 0.033465507 | 0.769730363 | 8.954554788 | 8.184824425 |
| 214021_x_at | ITGB5 | ENSG00000082781 | 0.033488347 | 1.43597723 | 6.631668819 | 5.19569159 |
| 227624_at | TET2 | ENSG00000168769 | 0.033488347 | 1.07690976 | 7.558846267 | 6.481936507 |
| 225093_at | UTRN | ENSG00000152818 | 0.033488347 | 1.565715389 | 10.04128739 | 8.475571999 |
| 227379_at | MBOAT1 | ENSG00000172197 | 0.033637219 | 1.226714416 | 7.197726855 | 5.971012439 |
| 213139_at | SNAI2 | ENSG00000019549 | 0.033800859 | 1.549581679 | 6.953320081 | 5.403738401 |
| 212099_at | RHOB | ENSG00000143878 | 0.033800859 | 1.563197657 | 9.851472658 | 8.288275001 |
| 204894_s_at | AOC3 | ENSG00000131471 | 0.033800859 | 1.488715186 | 6.950874216 | 5.46215903 |
| 219432_at | EVC | ENSG00000072840 | 0.033800859 | 0.973495437 | 7.657380766 | 6.683885329 |
| 55065_at | MARK4 | ENSG00000007047 | 0.033800859 | 0.450131769 | 7.655069099 | 7.204937331 |
| 226353_at | SPPL2A | ENSG00000138600 | 0.034000797 | 1.086036284 | 9.82423751 | 8.738201226 |
| 216598_s_at | CCL2 | ENSG00000108691 | 0.034000797 | 1.574100327 | 11.80437366 | 10.23027333 |
| 226568_at | FAM102B | ENSG00000162636 | 0.034000797 | 1.19493473 | 7.320320998 | 6.125386268 |
| 218764_at | PRKCH | ENSG00000027075 | 0.034090749 | 1.324224837 | 8.842467348 | 7.518242511 |
| 225984_at | PRKAA1 | ENSG00000132356 | 0.034117995 | 0.761625478 | 6.863441712 | 6.101816234 |
| 201542_at | SAR1A | ENSG00000079332 | 0.034117995 | 0.735645671 | 9.772431133 | 9.036785462 |
| 203455_s_at | SAT1 | ENSG00000130066 | 0.034117995 | 1.316520019 | 11.13299333 | 9.816473307 |
| 225922_at | FNIP2 | ENSG00000052795 | 0.034117995 | 1.519527523 | 8.166224411 | 6.646696888 |
| 218665_at | FZD4 | ENSG00000174804 | 0.034117995 | 1.021163189 | 7.416230764 | 6.395067575 |
| 221666_s_at | PYCARD | ENSG00000103490 | 0.034190831 | 0.606108157 | 9.397235781 | 8.791127624 |
| 201058_s_at | MYL9 | ENSG00000101335 | 0.034190831 | 1.2676991 | 10.37627198 | 9.108572878 |
| 203397_s_at | GALNT3 | ENSG00000115339 | 0.034190831 | 1.306640305 | 5.45104883 | 4.144408525 |
| 222453_at | CYBRD1 | ENSG00000071967 | 0.034190831 | 1.315727811 | 9.001391772 | 7.685663961 |
| 208146_s_at | CPVL | ENSG00000106066 | 0.034190831 | 1.943264679 | 9.534937034 | 7.591672354 |
| 227334_at | USP54 | ENSG00000166348 | 0.034190831 | 0.595004786 | 7.632456518 | 7.037451732 |
| 226534_at | KITLG | ENSG00000049130 | 0.034190831 | 1.687120279 | 6.504808499 | 4.81768822 |
| 229120_s_at | CDC42SE1 | ENSG00000197622 | 0.034190831 | 1.026120843 | 9.039741792 | 8.013620949 |
| 225066_at | PPP2R2D | ENSG00000175470 | 0.034190831 | 0.410023152 | 6.51512502 | 6.105101868 |
| 211977_at | GPR107 | ENSG00000148358 | 0.034190831 | 0.41506351 | 7.323330309 | 6.908266799 |
| 217967_s_at | FAM129A | ENSG00000135842 | 0.034190831 | 1.631617618 | 9.361851904 | 7.730234286 |
| 207705_s_at | NINL | ENSG00000101004 | 0.034190831 | 0.458032479 | 7.469271682 | 7.011239203 |
| 230029_x_at | UBR3 | ENSG00000144357 | 0.034212391 | 0.713232741 | 7.406813979 | 6.693581237 |
| 227236_at | TSPAN2 | ENSG00000134198 | 0.034240086 | 1.160525883 | 6.827257615 | 5.666731733 |
| 202510_s_at | TNFAIP2 | ENSG00000185215 | 0.034240086 | 1.611311545 | 10.98695296 | 9.375641416 |
| 202450_s_at | CTSK | ENSG00000143387 | 0.034333589 | 2.058768294 | 10.34330275 | 8.284534455 |
| 226395_at | HOOK3 | ENSG00000168172 | 0.034360811 | 0.907384172 | 9.099732802 | 8.192348631 |
| 219403_s_at | HPSE | ENSG00000173083 | 0.034374363 | 1.273377491 | 7.073411349 | 5.800033858 |
| 227623_at | CACNA2D1 | ENSG00000153956 | 0.034401348 | 1.509374159 | 5.523191348 | 4.013817189 |
| 202239_at | PARP4 | ENSG00000102699 | 0.034401348 | 0.920213526 | 9.568361079 | 8.648147553 |
| 204646_at | DPYD | ENSG00000188641 | 0.034480931 | 1.716538106 | 8.184742 | 6.468203894 |
| 209335_at | DCN | ENSG00000011465 | 0.034606801 | 2.0562205 | 8.106748111 | 6.050527611 |
| 202341_s_at | TRIM2 | ENSG00000109654 | 0.034606801 | 1.154998273 | 7.682678312 | 6.527680038 |
| 203386_at | TBC1D4 | ENSG00000136111 | 0.034611968 | 1.59863556 | 9.927925002 | 8.329289442 |
| 202465_at | PCOLCE | ENSG00000106333 | 0.034687227 | 1.110793856 | 9.858341328 | 8.747547472 |
| 218501_at | ARGHEF3 | ENSG00000163947 | 0.034710054 | 1.211901006 | 9.345778775 | 8.433877769 |
| 203935_at | ACVR1 | ENSG00000115170 | 0.034710054 | 0.929748525 | 7.592939572 | 6.663191047 |
| 227726_at | RNF166 | ENSG00000158717 | 0.034778201 | 0.765255386 | 6.490241897 | 5.724986511 |
| 225351_at | FAM45A | ENSG00000119979 | 0.034778201 | 0.712687022 | 7.985558444 | 7.272871422 |
| 209967_s_at | CREM | ENSG00000095794 | 0.034778201 | 1.498708212 | 8.008162888 | 6.509454676 |
| 217767_at | C3 | ENSG00000125730 | 0.034901896 | 1.610302964 | 11.06255643 | 9.452253469 |
| 202377_at | LEPROT | ENSG00000213625 | 0.034901896 | 0.958758279 | 8.927718442 | 7.968960164 |
| 212067_s_at | C1R | ENSG00000159403 | 0.034901896 | 1.095883775 | 10.97729778 | 9.881414002 |
| 206461_x_at | MT1H | ENSG00000205358 | 0.034906504 | 0.937479848 | 10.94110484 | 10.00362499 |
| 212651_at | RHOBTB1 | ENSG00000072422 | 0.034920435 | 1.278051787 | 5.30325947 | 4.025207683 |
| 213077_at | YTHDC2 | ENSG00000047188 | 0.034920435 | 1.082274718 | 7.160912963 | 6.078638245 |
| 238617_at | KIF26B | ENSG00000162849 | 0.034920435 | 1.816726723 | 7.548086863 | 5.73136014 |
| 201540_at | FHL1 | ENSG00000022267 | 0.034920435 | 2.090914464 | 8.481468308 | 6.390553844 |
| 227325_at | PRR24 | ENSG00000257704 | 0.034920435 | 0.590098253 | 7.823937835 | 7.233839582 |
| 208093_s_at | NDEL1 | ENSG00000166579 | 0.034947401 | 0.741034838 | 8.576080766 | 7.835045928 |
| 202006_at | PTPN12 | ENSG00000127947 | 0.035038557 | 1.018262804 | 9.540273742 | 8.522010938 |
| 209216_at | WDR45 | ENSG00000196998 | 0.035038557 | 0.696795004 | 8.826888223 | 8.130093219 |
| 225782_at | MSRB3 | ENSG00000174099 | 0.035038557 | 1.444375496 | 6.51471588 | 5.070340384 |
| 200602_at | APP | ENSG00000142912 | 0.035038557 | 1.325930012 | 9.055534642 | 7.729604631 |
| 226939_at | CPEB2 | ENSG00000137449 | 0.035264082 | 1.226498712 | 7.611624387 | 6.385125675 |
| 218986_s_at | DDX60 | ENSG00000137628 | 0.035316356 | 1.486021371 | 8.318531076 | 6.832509705 |
| 203139_at | DAPK1 | ENSG00000196730 | 0.035316356 | 1.707948936 | 8.912548269 | 7.204599333 |
| 213764_s_at | MFAP5 | ENSG00000197614 | 0.035434896 | 1.297982185 | 5.790529452 | 4.492547267 |
| 202565_s_at | SVIL | ENSG00000197321 | 0.035639458 | 1.389051153 | 8.635444721 | 7.246393568 |
| 210968_s_at | RTN4 | ENSG00000115310 | 0.035770121 | 0.829593112 | 10.69653249 | 9.866939383 |
| 225562_at | RASA3 | ENSG00000185989 | 0.035779896 | 0.972127539 | 9.062084532 | 8.089956993 |
| 218454_at | PLBD1 | ENSG00000121316 | 0.03578637 | 1.441982401 | 10.25391754 | 8.81193514 |
| 209467_s_at | MKNK1 | ENSG00000079277 | 0.035868055 | 0.783294393 | 8.294847035 | 7.511552642 |
| 226869_at | MEGF6 | ENSG00000162591 | 0.035950269 | 1.265043406 | 7.859101342 | 6.594057936 |
| 221569_at | AHI1 | ENSG00000135541 | 0.035950269 | 1.076140591 | 7.793652098 | 6.717511506 |
| 1569157_s_at | ZNF846 | ENSG00000196605 | 0.035950269 | 0.774623333 | 6.057018085 | 5.282394752 |
| 218132_s_at | TSEN34 | ENSG00000170892 | 0.035950269 | 0.438866613 | 8.237398535 | 7.798531921 |
| 201591_s_at | NISCH | ENSG00000010322 | 0.036417437 | 0.405648171 | 8.996513107 | 8.590864936 |
| 227098_at | DUSP18 | ENSG00000167065 | 0.036417437 | 0.482163643 | 7.203923142 | 6.721759499 |
| 203910_at | ARHGAP29 | ENSG00000137962 | 0.036417437 | 1.513169879 | 7.336341356 | 5.823171477 |
| 1555847_a_at | LOC284454 | NA | 0.036417437 | 0.929013425 | 9.021958303 | 8.092944878 |
| 241353_s_at | LOC100507507 | NA | 0.036417437 | 0.563123987 | 6.938191846 | 6.375067858 |
| 202225_at | CRK | ENSG00000167193 | 0.036417437 | 0.76604843 | 9.380487824 | 8.614439395 |
| 212915_at | PDZRN3 | ENSG00000121440 | 0.036490985 | 1.312579319 | 6.719045192 | 5.406465873 |
| 202920_at | ANK2 | ENSG00000145362 | 0.036490985 | 1.463394123 | 8.12462823 | 6.661234107 |
| 235391_at | FAM92A1 | ENSG00000188343 | 0.036548329 | 0.621136179 | 7.223438844 | 6.602302665 |
| 224906_at | ANO6 | ENSG00000177119 | 0.036675313 | 0.877112362 | 8.66468344 | 7.787571078 |
| 227087_at | INPP4A | ENSG00000040933 | 0.036675313 | 1.023789448 | 8.031874294 | 7.005084846 |
| 225931_s_at | RNF213 | ENSG00000173821 | 0.036802205 | 1.10725232 | 9.749178104 | 8.641925784 |
| 212690_at | DDHD2 | ENSG00000085788 | 0.036831284 | 0.878356205 | 9.176866822 | 8.298510616 |
| 209003_at | SLC25A11 | ENSG00000108528 | 0.036890888 | 0.43772317 | 7.954667222 | 7.516944052 |
| 202206_at | ARL4C | ENSG00000188042 | 0.036890888 | 1.389805117 | 9.321386817 | 7.9315817 |
| 232000_at | TTC39B | ENSG00000155158 | 0.037068478 | 1.356512637 | 4.978732932 | 3.622220295 |
| 225386_s_at | HNRPLL | NA | 0.037068478 | 1.459395359 | 9.121601553 | 7.662206194 |
| 226409_at | TBC1D20 | ENSG00000125875 | 0.037068478 | 0.505585753 | 8.965323908 | 8.459738154 |
| 204066_s_at | AGAP1 | ENSG00000157985 | 0.037087501 | 1.078941551 | 6.075080058 | 4.996138507 |
| 212441_at | KIAA0232 | ENSG00000170871 | 0.037087501 | 0.590210466 | 8.606785896 | 8.01657543 |
| 225726_s_at | PLEKHH1 | ENSG00000054690 | 0.037087501 | 0.952695106 | 5.964957706 | 5.0122626 |
| 232094_at | KATNBL1 | ENSG00000134152 | 0.037087501 | 0.5971703 | 7.264443135 | 6.667272835 |
| 225604_s_at | GLIPR2 | ENSG00000122694 | 0.037323559 | 0.853910711 | 7.844159209 | 6.990248499 |
| 205225_at | ESR1 | ENSG00000091831 | 0.037323559 | 1.382468278 | 6.467921229 | 5.085452951 |
| 218838_s_at | TTC31 | ENSG00000115282 | 0.037323559 | 0.672650224 | 8.524158479 | 7.851508256 |
| 227961_at | CTSB | ENSG00000164733 | 0.037323559 | 1.338139585 | 10.24117448 | 8.903034892 |
| 1558041_a_at | KIAA0895L | ENSG00000196123 | 0.037412554 | 0.876742459 | 7.925106663 | 7.048364204 |
| 209540_at | IGF1 | ENSG00000017427 | 0.037412554 | 1.522509458 | 8.256633376 | 6.734123918 |
| 203651_at | ZFYVE16 | ENSG00000039319 | 0.037412554 | 1.025141601 | 8.527484121 | 7.502342519 |
| 224900_at | ANKFY1 | ENSG00000185722 | 0.037546087 | 0.532952956 | 8.581470479 | 8.048517523 |
| 223441_at | SLC17A5 | ENSG00000119899 | 0.037627191 | 0.645005952 | 6.824616025 | 6.179610072 |
| 204294_at | AMT | ENSG00000145020 | 0.037627191 | 0.399434215 | 8.335213696 | 7.935779481 |
| 204040_at | RNF144A | ENSG00000151692 | 0.037681586 | 1.481347774 | 6.933003595 | 5.451655821 |
| 225272_at | SAT2 | ENSG00000141504 | 0.037706134 | 0.618612094 | 9.078150553 | 8.459538459 |
| 204464_s_at | EDNRA | ENSG00000151617 | 0.037706134 | 1.424834074 | 7.176770385 | 8.751936311 |
| 205011_at | VWA5A | ENSG00000110002 | 0.037706134 | 1.501068828 | 8.178126937 | 6.677058109 |
| 212765_at | CAMSAP2 | ENSG00000118200 | 0.037713812 | 1.070277586 | 7.585165033 | 6.514887447 |
| 212624_s_at | CHN1 | ENSG00000128656 | 0.037754756 | 1.199366569 | 8.339419696 | 7.140053127 |
| 209213_at | CBR1 | ENSG00000159228 | 0.037828214 | 0.716339962 | 9.378175666 | 8.661835705 |
| 207738_s_at | NCKAP1 | ENSG00000061676 | 0.037828214 | 1.742016807 | 5.909031575 | 4.167014768 |
| 206856_at | LILRB5 | ENSG00000105609 | 0.037950057 | 1.58731928 | 8.757091921 | 7.169772641 |
| 200697_at | HK1 | ENSG00000156515 | 0.03798879 | 0.497080421 | 10.98016503 | 10.48308461 |
| 219134_at | ELTD1 | ENSG00000162618 | 0.038039799 | 1.633116993 | 7.99727279 | 6.364155796 |
| 222294_s_at | RAB27A | ENSG00000069974 | 0.038154279 | 1.512875635 | 7.227372685 | 5.71449705 |
| 201057_s_at | GOLGB1 | ENSG00000173230 | 0.038450193 | 0.639618056 | 9.22083416 | 8.581216104 |
| 218017_s_at | HGSNAT | ENSG00000165102 | 0.038566561 | 1.101893048 | 8.583362968 | 7.48146992 |
| 226905_at | FAM101B | ENSG00000183688 | 0.038659566 | 1.355638698 | 8.83238214 | 7.476743442 |
| 226616_s_at | NDUFV3 | ENSG00000160194 | 0.038671508 | 0.66206984 | 10.46600414 | 9.803934298 |
| 215127_s_at | RBMS1 | ENSG00000153250 | 0.038707721 | 0.985761408 | 9.060146035 | 8.074384626 |
| 212373_at | FEM1B | ENSG00000169018 | 0.03889702 | 0.961868668 | 8.42816521 | 7.466296542 |
| 222750_s_at | SRD5A3 | ENSG00000128039 | 0.03889702 | 0.762605382 | 8.2518174 | 7.489212018 |
| 236006_s_at | AKAP10 | ENSG00000108599 | 0.069007321 | 0.655037688 | 7.858287091 | 7.173249402 |
| 224733_at | CMTM3 | ENSG00000140931 | 0.039181923 | 0.690269684 | 9.982739003 | 9.292469319 |
| 204981_at | SLC22A18 | ENSG00000110628 | 0.039181923 | 0.561183888 | 0.561183888 | 7.028755911 |
| 223077_at | TMOD3 | ENSG00000138594 | 0.039239917 | 1.092206647 | 7.964720713 | 6.872514066 |
| 209686_at | S100B | ENSG00000160307 | 0.039241475 | 0.901290569 | 6.761868136 | 5.860577567 |
| 225325_at | MFSD6 | ENSG00000151690 | 0.039241475 | 0.839407884 | 8.69192971 | 7.852521826 |
| 205236_x_at | SOD3 | ENSG00000109610 | 0.03957993 | 0.891900627 | 8.45163302 | 7.559732393 |
| 228220_at | FCHO2 | ENSG00000157107 | 0.03957993 | 1.563762465 | 8.795856173 | 7.232093708 |
| 205904_at | MICA | ENSG00000204520 | 0.03957993 | 0.427228475 | 7.684753887 | 7.257525412 |
| 226777_at | ADAM12 | ENSG00000148848 | 0.03957993 | 1.943247065 | 7.771445066 | 5.828198001 |
| 213618_at | ARAP2 | ENSG00000047365 | 0.03957993 | 1.028082071 | 7.620559319 | 6.592477248 |
| 205624_at | CPA3 | ENSG00000163751 | 0.03957993 | 2.151198872 | 6.492961642 | 4.34176277 |
| 215268_at | KIAA0754 | ENSG00000255103 | 0.03957993 | 0.809104773 | 6.285140538 | 5.476035765 |
| 203672_x_at | TPMT | ENSG00000137364 | 0.03957993 | 0.554375735 | 9.623390267 | 9.069014532 |
| 1555229_a_at | C1S | ENSG00000182326 | 0.03957993 | 1.562833271 | 10.46271064 | 8.899877365 |
| 203855_at | WDR47 | ENSG00000085433 | 0.03957993 | 0.715002164 | 7.128000627 | 6.412998464 |
| 207072_at | IL18RAP | ENSG00000115607 | 0.03957993 | 0.981690585 | 7.548943432 | 6.567252847 |
| 221935_s_at | EOGT | ENSG00000163378 | 0.03960808 | 0.83587661 | 7.127103969 | 6.291227359 |
| 203232_s_at | ATXN1 | ENSG00000124788 | 0.03960808 | 1.486472653 | 8.293823116 | 6.807350464 |
| 202096_s_at | TSPO | ENSG00000100300 | 0.039769597 | 0.618613605 | 9.561862207 | 8.942972602 |
| 224813_at | WASL | ENSG00000106299 | 0.039769597 | 0.810234478 | 8.822272437 | 8.012037959 |
| 213455_at | FAM114A1 | ENSG00000197712 | 0.039797292 | 0.841637956 | 7.947071796 | 7.10543384 |
| 226873_at | FAM63B | ENSG00000128923 | 0.039877715 | 0.781681687 | 7.054055786 | 6.272374099 |
| 1554503_a_at | OSCAR | ENSG00000170909 | 0.040082873 | 0.481594381 | 6.652982594 | 6.171388213 |
| 206118_at | STAT4 | ENSG00000138378 | 0.040381182 | 1.642843342 | 7.76374389 | 6.120900548 |
| 210970_s_at | IBTK | ENSG00000005700 | 0.040543289 | 0.753886475 | 8.260872375 | 7.506985901 |
| 227113_at | ADHFE1 | ENSG00000147576 | 0.040543289 | 0.706136194 | 8.382822434 | 7.67668624 |
| 202949_s_at | FHL2 | ENSG00000115641 | 0.040593602 | 1.403182627 | 7.613598294 | 6.210415667 |
| 225579_at | PQLC3 | ENSG00000162976 | 0.040596862 | 1.113667847 | 9.537872547 | 8.4242047 |
| 235291_s_at | FLJ32255 | NA | 0.040715697 | 1.119695897 | 7.867314072 | 6.747618175 |
| 203939_at | NT5E | ENSG00000135318 | 0.040733387 | 0.806179248 | 7.42523104 | 6.619051792 |
| 214274_s_at | ACAA1 | ENSG00000060971 | 0.040793718 | 0.502419076 | 9.465451705 | 8.963032628 |
| 204046_at | PLCB2 | ENSG00000137841 | 0.040802463 | 0.533783551 | 8.533797162 | 8.000013611 |
| 202392_s_at | PISD | ENSG00000241878 | 0.041073186 | 0.356364153 | 8.566583027 | 8.210218874 |
| 218326_s_at | LGR4 | ENSG00000205213 | 0.041073186 | 1.255121429 | 5.022109926 | 3.766988497 |
| 229119_s_at | ZSWIM7 | ENSG00000214941 | 0.041073186 | 1.024786314 | 7.910530196 | 6.885743882 |
| 206491_s_at | NAPA | ENSG00000105402 | 0.041414647 | 0.495436416 | 9.333764247 | 8.838327831 |
| 227450_at | ERP27 | ENSG00000139055 | 0.041414647 | 0.966196682 | 6.328914416 | 5.362717734 |
| 200866_s_at | PSAP | ENSG00000197746 | 0.041414647 | 1.085359838 | 11.69848738 | 10.61312755 |
| 200720_s_at | ACTR1A | ENSG00000138107 | 0.041414647 | 0.496753402 | 8.824861676 | 8.328108274 |
| 232064_at | FER | ENSG00000151422 | 0.041414647 | 0.656054594 | 7.136386009 | 6.480331415 |
| 217922_at | MAN1A2 | ENSG00000198162 | 0.04144504 | 0.69736844 | 8.202283201 | 7.504914761 |
| 201944_at | HEXB | ENSG00000049860 | 0.041569794 | 1.010944746 | 11.59320288 | 10.58225814 |
| 210986_s_at | TPM1 | ENSG00000140416 | 0.041569794 | 1.650337469 | 9.944608224 | 8.294270755 |
| 227568_at | HECTD2 | ENSG00000165338 | 0.041569794 | 1.119931753 | 6.685060885 | 5.565129131 |
| 200645_at | GABARAP | ENSG00000170296 | 0.041569794 | 0.631747342 | 11.38104358 | 10.74929624 |
| 219892_at | TM6SF1 | ENSG00000136404 | 0.041569794 | 1.518719736 | 7.818958694 | 6.300238959 |
| 222431_at | SPIN1 | ENSG00000106723 | 0.041569794 | 0.70282485 | 9.186280228 | 8.483455378 |
| 203665_at | HMOX1 | ENSG00000100292 | 0.041569794 | 1.019908548 | 9.506813677 | 8.486905129 |
| 202506_at | SSFA2 | ENSG00000138434 | 0.041569794 | 0.947553911 | 8.018471473 | 7.070917562 |
| 221899_at | N4BP2L2 | ENSG00000244754 | 0.041569794 | 0.865335269 | 9.046774211 | 8.181438942 |
| 203668_at | MAN2C1 | ENSG00000140400 | 0.041690206 | 0.633916604 | 8.663669977 | 8.029753373 |
| 206295_at | IL18 | ENSG00000150782 | 0.041916833 | 1.292174393 | 9.298735781 | 8.006561388 |
| 228152_s_at | DDX60L | ENSG00000181381 | 0.041916833 | 1.421343576 | 7.853791688 | 6.432448112 |
| 228341_at | NUDT16 | ENSG00000198585 | 0.041916833 | 0.813285601 | 6.707601846 | 5.894316245 |
| 218164_at | SPATA20 | ENSG00000006282 | 0.041916833 | 0.587929592 | 9.02333374 | 8.435404148 |
| 227070_at | GLT8D2 | ENSG00000120820 | 0.041916833 | 1.757351115 | 5.840317565 | 4.082966449 |
| 213083_at | SLC35D2 | ENSG00000130958 | 0.041916833 | 0.829365427 | 7.315854336 | 6.486488909 |
| 224772_at | NAV1 | ENSG00000134369 | 0.041916833 | 1.051478872 | 7.602145754 | 6.550666882 |
| 229287_at | PCNX | ENSG00000100731 | 0.041916833 | 0.943930482 | 7.566201547 | 6.622271065 |
| 201200_at | CREG1 | ENSG00000143162 | 0.041916833 | 1.058155445 | 11.10068635 | 10.0425309 |
| 229450_at | IFIT3 | ENSG00000119917 | 0.041916833 | 1.6631975 | 9.084234826 | 7.421037327 |
| 228249_at | C11orf74 | ENSG00000166352 | 0.041916833 | 0.848798181 | 5.174338696 | 4.325540515 |
| 38487_at | STAB1 | ENSG00000010327 | 0.04205946 | 1.090580398 | 9.310990038 | 8.22040964 |
| 202974_at | MPP1 | ENSG00000130830 | 0.04205946 | 1.062976134 | 9.614559777 | 8.551583643 |
| 226510_at | HEATR5A | ENSG00000129493 | 0.04205946 | 0.7510398 | 7.25960589 | 6.50872079 |
| 228282_at | MFSD8 | ENSG00000164073 | 0.04205946 | 0.819273204 | 7.582661234 | 6.763388029 |
| 203675_at | NUCB2 | ENSG00000070081 | 0.04205946 | 0.905774662 | 9.751698376 | 8.845923714 |
| 228384_s_at | PYROXD2 | ENSG00000119943 | 0.04205946 | 0.304011115 | 6.385890727 | 6.081879612 |
| 229367_s_at | GIMAP6 | ENSG00000133561 | 0.04205946 | 1.409013647 | 8.888667615 | 7.479653968 |
| 218309_at | CAMK2N1 | ENSG00000162545 | 0.04205946 | 1.260533336 | 7.868836571 | 6.608303235 |
| 34726_at | CACNB3 | ENSG00000167535 | 0.04205946 | 0.413630799 | 6.934617087 | 6.520986288 |
| 212670_at | ELN | ENSG00000049540 | 0.04205946 | 1.231152927 | 8.81370025 | 7.582547323 |
| 229800_at | DCLK1 | ENSG00000133083 | 0.04205946 | 1.498592436 | 6.412070979 | 4.913478542 |
| 201975_at | CLIP1 | ENSG00000130779 | 0.042246192 | 1.028511221 | 8.482482941 | 7.453971719 |
| 224413_s_at | TM2D2 | ENSG00000169490 | 0.042247538 | 0.61300573 | 9.024042453 | 8.411036723 |
| 204011_at | SPRY2 | ENSG00000136158 | 0.042331876 | 1.318852631 | 7.198128454 | 5.879275822 |
| 235033_at | NPEPL1 | ENSG00000215440 | 0.042331876 | 0.763205701 | 5.890585229 | 5.127379527 |
| 228348_at | LINS | ENSG00000140471 | 0.042350925 | 0.889830233 | 8.824668316 | 7.934838083 |
| 227628_at | GPX8 | ENSG00000164294 | 0.042351094 | 1.180447997 | 6.970795245 | 5.790347248 |
| 1557112_a_at | VPS53 | ENSG00000141252 | 0.042351094 | 0.518032516 | 7.975808016 | 7.4577755 |
| 228071_at | GIMAP7 | ENSG00000179144 | 0.042351094 | 1.647364043 | 8.992108891 | 7.344744848 |
| 209264_s_at | TSPAN4 | ENSG00000214063 | 0.042351094 | 0.681754755 | 9.006699575 | 8.32494482 |
| 208892_s_at | DUSP6 | ENSG00000139318 | 0.042351094 | 1.228973817 | 7.992040123 | 6.763066305 |
| 201315_x_at | IFITM2 | ENSG00000185201 | 0.042351094 | 0.739392199 | 11.67358479 | 10.9341926 |
| 222802_at | EDN1 | ENSG00000078401 | 0.042351094 | 1.460541762 | 6.322746092 | 4.86220433 |
| 1553395_a_at | CD200R1 | ENSG00000163606 | 0.042351094 | 1.256910265 | 6.464778546 | 5.207868281 |
| 226399_at | DNAJB14 | ENSG00000164031 | 0.042351094 | 0.849947729 | 8.162884294 | 7.312936565 |
| 226490_at | NHSL1 | ENSG00000135540 | 0.042351094 | 1.194665858 | 7.117630828 | 5.92296497 |
| 222513_s_at | SORBS1 | ENSG00000095637 | 0.042351094 | 1.325460985 | 8.112293431 | 6.786832446 |
| 218705_s_at | SNX24 | ENSG00000064652 | 0.042351094 | 0.90943161 | 7.531422028 | 6.621990418 |
| 226837_at | SPRED1 | ENSG00000166068 | 0.042351094 | 1.212871192 | 7.561561543 | 6.348690351 |
| 225338_at | ZYG11B | ENSG00000162378 | 0.042351094 | 0.624478143 | 7.313789874 | 6.689311731 |
| 204955_at | SRPX | ENSG00000101955 | 0.042351094 | 1.769176749 | 7.330012241 | 5.560835493 |
| 210840_s_at | IQGAP1 | ENSG00000140575 | 0.042351094 | 0.815667827 | 10.6038101 | 9.788142278 |
| 201494_at | PRCP | ENSG00000137509 | 0.042351094 | 0.880611665 | 10.84225026 | 9.961638596 |
| 204415_at | IFI6 | ENSG00000126709 | 0.042351094 | 1.113083585 | 9.36647792 | 8.253394336 |
| 209290_s_at | NFIB | ENSG00000147862 | 0.042351094 | 1.655774494 | 8.40919634 | 6.753421846 |
| 221474_at | MYL12B | ENSG00000118680 | 0.042419604 | 0.586131101 | 10.97279348 | 10.38666238 |
| 235747_at | SLC25A16 | ENSG00000122912 | 0.042419604 | 0.546049363 | 7.042744928 | 6.496695565 |
| 201924_at | AFF1 | ENSG00000172493 | 0.042419604 | 0.952063778 | 10.2416206 | 9.289556827 |
| 218045_x_at | PTMS | ENSG00000159335 | 0.042596049 | 0.607162357 | 9.924387924 | 9.317225567 |
| 213943_at | TWIST1 | ENSG00000122691 | 0.042596049 | 1.16873421 | 7.271938017 | 6.103203807 |
| 226752_at | FAM174A | ENSG00000174132 | 0.042596049 | 0.92871631 | 5.776448605 | 4.847732296 |
| 201876_at | PON2 | ENSG00000105854 | 0.042719768 | 1.187571081 | 9.054738216 | 7.867167135 |
| 1554240_a_at | ITGAL | ENSG00000005844 | 0.042881947 | 1.302505376 | 9.317162745 | 8.014657369 |
| 203989_x_at | F2R | ENSG00000181104 | 0.042891128 | 1.246519921 | 7.440038684 | 6.193518762 |
| 226425_at | CLIP4 | ENSG00000115295 | 0.042891128 | 1.499860122 | 7.783328081 | 6.28346796 |
| 200762_at | DPYSL2 | ENSG00000092964 | 0.042891128 | 1.159171793 | 9.328123535 | 8.168951742 |
| 223681_s_at | INADL | ENSG00000132849 | 0.042891128 | 1.250998756 | 6.181876637 | 4.930877881 |
| 211986_at | AHNAK | ENSG00000124942 | 0.042891128 | 1.334146601 | 11.47855827 | 10.14441167 |
| 204112_s_at | HNMT | ENSG00000150540 | 0.042891128 | 1.303474995 | 8.681932953 | 7.378457958 |
| 235360_at | PLEKHM3 | ENSG00000178385 | 0.042891128 | 0.436326441 | 7.481077573 | 7.044751132 |
| 209276_s_at | GLRX | ENSG00000173221 | 0.042939851 | 1.153352768 | 10.46513253 | 9.311779765 |
| 226113_at | ZNF436 | ENSG00000125945 | 0.042950747 | 0.967635125 | 7.934452645 | 6.96681752 |
| 201694_at | EGR1 | ENSG00000120738 | 0.04303339 | 2.356208235 | 9.680723259 | 7.324515023 |
| 204417_at | GALC | ENSG00000054983 | 0.04303339 | 1.18197166 | 9.186049316 | 8.004077656 |
| 211178_s_at | PSTPIP1 | ENSG00000140368 | 0.043103445 | 0.558040078 | 8.764374999 | 8.206334921 |
| 203088_at | FBLN5 | ENSG00000140092 | 0.043103445 | 1.57461041 | 8.128331591 | 6.553721182 |
| 218450_at | HEBP1 | ENSG00000013583 | 0.043103445 | 0.756321096 | 8.459513001 | 7.703191905 |
| 210113_s_at | NLRP1 | ENSG00000091592 | 0.043127824 | 0.654465183 | 7.398936818 | 6.744471635 |
| 225673_at | MYADM | ENSG00000179820 | 0.043127824 | 1.185973479 | 9.460657173 | 8.274683693 |
| 31874_at | GAS2L1 | ENSG00000185340 | 0.043127824 | 0.959125758 | 7.063627768 | 6.10450201 |
| 222597_at | SNAP29 | ENSG00000099940 | 0.043127824 | 0.523137969 | 8.682269428 | 8.159131459 |
| 226279_at | PRSS23 | ENSG00000150687 | 0.043152778 | 1.36043671 | 7.67513518 | 6.31469847 |
| 235570_at | RBMS3 | ENSG00000144642 | 0.043152778 | 1.782327906 | 7.335632998 | 5.553305092 |
| 214830_at | SLC38A6 | ENSG00000139974 | 0.043249838 | 1.276930786 | 7.341342657 | 6.064411871 |
| 201366_at | ANXA7 | ENSG00000138279 | 0.043259906 | 0.763011677 | 8.850377121 | 8.087365443 |
| 203179_at | GALT | ENSG00000213930 | 0.043316491 | 0.410171519 | 8.83328444 | 8.423112921 |
| 225919_s_at | C9orf72 | ENSG00000147894 | 0.04347548 | 0.855251908 | 8.181409697 | 7.326157789 |
| 36711_at | MAFF | ENSG00000185022 | 0.04347548 | 1.042386023 | 5.605979018 | 4.563592995 |
| 205174_s_at | QPCT | ENSG00000115828 | 0.043531412 | 1.261724497 | 8.418598958 | 7.156874461 |
| 225032_at | FNDC3B | ENSG00000075420 | 0.043656579 | 1.148111719 | 9.960617367 | 8.542505648 |
| 225283_at | ARRDC4 | ENSG00000140450 | 0.043675035 | 1.263277402 | 6.919418159 | 5.656140757 |
| 212820_at | DMXL2 | ENSG00000104093 | 0.043693329 | 1.723755285 | 9.098753998 | 7.374998712 |
| 202411_at | IFI27 | ENSG00000165949 | 0.043693329 | 1.419904538 | 10.65301374 | 9.233109201 |
| 201444_s_at | ATP6AP2 | ENSG00000182220 | 0.043693329 | 0.848128217 | 9.927790973 | 9.079662756 |
| 212209_at | MED13L | ENSG00000123066 | 0.043693329 | 0.718689048 | 7.683203365 | 6.964514318 |
| 228082_at | CLMP | ENSG00000166250 | 0.043693329 | 0.811085985 | 8.019360923 | 7.208274938 |
| 201579_at | FAT1 | ENSG00000083857 | 0.043693329 | 1.199696602 | 6.984437994 | 5.784741393 |
| 201050_at | PLD3 | ENSG00000105223 | 0.043738675 | 0.872712957 | 11.22150754 | 10.34879458 |
| 205726_at | DIAPH2 | ENSG00000147202 | 0.043865363 | 0.824323825 | 7.524441934 | 6.700118109 |
| 212717_at | PLEKHM1 | ENSG00000225190 | 0.04386943 | 0.350082673 | 8.468551352 | 8.118468679 |
| 202827_s_at | MMP14 | ENSG00000157227 | 0.043950069 | 1.093430274 | 9.702737954 | 8.60930768 |
| 201594_s_at | PPP4R1 | ENSG00000154845 | 0.043950069 | 0.81803938 | 9.27411864 | 8.456079261 |
| 203460_s_at | PSEN1 | ENSG00000080815 | 0.043950069 | 0.81390235 | 8.845796903 | 8.031894553 |
| 221840_at | PTPRE | ENSG00000132334 | 0.044016553 | 1.294850625 | 8.885637792 | 7.590787167 |
| 203410_at | AP3M2 | ENSG00000070718 | 0.044042681 | 0.768339069 | 7.036583 | 6.268243931 |
| 226582_at | LOC400043 | NA | 0.044042681 | 0.869479209 | 6.73443097 | 5.864951761 |
| 209600_s_at | ACOX1 | ENSG00000161533 | 0.044042681 | 0.648332519 | 8.512058318 | 7.863725799 |
| 221814_at | GPR124 | ENSG00000020181 | 0.044042681 | 1.095880575 | 8.488647675 | 7.3927671 |
| 230836_at | ST8SIA4 | ENSG00000113532 | 0.044042681 | 1.017497807 | 7.57919038 | 6.561692573 |
| 204270_at | SKI | ENSG00000157933 | 0.044042681 | 0.896035069 | 9.220481497 | 8.324446427 |
| 200872_at | S100A10 | ENSG00000197747 | 0.044042681 | 0.862231392 | 12.14558315 | 11.28335176 |
| 219761_at | CLEC1A | ENSG00000150048 | 0.044042681 | 1.291289338 | 6.704345965 | 5.413056627 |
| 212708_at | MSL1 | ENSG00000188895 | 0.044042681 | 0.856069768 | 9.529726222 | 8.673656454 |
| 204326_x_at | MT1X | ENSG00000187193 | 0.044070216 | 1.001729342 | 10.8648548 | 9.863125458 |
| 241392_at | TMEM39A | ENSG00000176142 | 0.044070216 | 0.341471009 | 6.928601856 | 6.587130847 |
| 205771_s_at | AKAP7 | ENSG00000118507 | 0.044070216 | 0.731995834 | 6.526724778 | 5.794728944 |
| 206227_at | CILP | ENSG00000138615 | 0.044070216 | 2.257502766 | 8.735997724 | 6.478494958 |
| 204158_s_at | TCIRG1 | ENSG00000110719 | 0.044070216 | 0.593079771 | 9.597157124 | 9.004077354 |
| 212948_at | CAMTA2 | ENSG00000108509 | 0.044070216 | 0.538388211 | 9.486336458 | 8.947948247 |
| 218241_at | GOLGA5 | ENSG00000066455 | 0.044078355 | 0.503478348 | 7.912463839 | 7.408985491 |
| 203042_at | LAMP2 | ENSG00000005893 | 0.044078355 | 1.386613769 | 9.189891489 | 7.80327772 |
| 223264_at | MESDC1 | ENSG00000140406 | 0.044215499 | 0.658578285 | 8.66869473 | 8.010116444 |
| 243141_at | SGMS2 | ENSG00000164023 | 0.044312646 | 1.085634594 | 5.816519276 | 4.730884682 |
| 212513_s_at | USP33 | ENSG00000077254 | 0.044312646 | 0.869205809 | 8.910601155 | 8.041395346 |
| 44111_at | VPS33B | ENSG00000184056 | 0.044312646 | 0.71852938 | 7.769682095 | 7.051152715 |
| 203044_at | CHSY1 | ENSG00000131873 | 0.044778087 | 0.860312924 | 9.374891415 | 8.514578491 |
| 201133_s_at | PJA2 | ENSG00000198961 | 0.045063391 | 0.928851445 | 8.789327346 | 7.860475902 |
| 238478_at | BNC2 | ENSG00000173068 | 0.04510855 | 1.581928937 | 6.500666236 | 4.918737299 |
| 207121_s_at | MAPK6 | ENSG00000069956 | 0.045216957 | 0.971961406 | 9.250325309 | 8.278363904 |
| 226757_at | IFIT2 | ENSG00000119922 | 0.045216957 | 1.258163287 | 7.398381051 | 6.140217764 |
| 224929_at | TMEM173 | ENSG00000184584 | 0.045216957 | 0.976901806 | 8.795661513 | 7.818759707 |
| 219013_at | GALNT11 | ENSG00000178234 | 0.045216957 | 0.624765223 | 8.111616397 | 7.486851174 |
| 203732_at | TRIP4 | ENSG00000103671 | 0.045216957 | 0.534991672 | 8.282763586 | 7.747771914 |
| 206991_s_at | CCR5 | ENSG00000160791 | 0.045321132 | 1.498899795 | 8.807195729 | 7.308295934 |
| 212192_at | KCTD12 | ENSG00000179695 | 0.045321132 | 1.241111969 | 11.04261631 | 9.801504337 |
| 212071_s_at | SPTBN1 | ENSG00000115306 | 0.045389085 | 1.148298747 | 10.50838671 | 9.360087962 |
| 204194_at | BACH1 | ENSG00000156273 | 0.045389085 | 0.684495403 | 7.331339629 | 6.646844226 |
| 213469_at | PGAP1 | ENSG00000197121 | 0.045389085 | 0.801387927 | 4.677932308 | 3.876544381 |
| 207173_x_at | CDH11 | ENSG00000140937 | 0.045389085 | 1.855491998 | 9.112092659 | 7.256600661 |
| 202020_s_at | LANCL1 | ENSG00000115365 | 0.045389085 | 0.72258943 | 9.184946138 | 8.462356708 |
| 201384_s_at | NBR1 | ENSG00000188554 | 0.045622403 | 0.69719383 | 9.452833592 | 8.755639763 |
| 213004_at | ANGPTL2 | ENSG00000136859 | 0.045650379 | 1.488271363 | 9.435206476 | 7.946935113 |
| 203310_at | STXBP3 | ENSG00000116266 | 0.045775429 | 0.770798736 | 7.72163929 | 6.950840554 |
| 212239_at | PIK3R1 | ENSG00000145675 | 0.045775429 | 0.737750823 | 9.025386358 | 8.287635535 |
| 225864_at | FAM84B | ENSG00000168672 | 0.045775429 | 1.677914651 | 6.194231305 | 4.516316654 |
| 218679_s_at | VPS28 | ENSG00000160948 | 0.045809883 | 0.390907854 | 9.961259817 | 9.300351963 |
| 211964_at | COL4A2 | ENSG00000134871 | 0.045856854 | 1.232811948 | 10.70556132 | 9.472749367 |
| 212501_at | CEBPB | ENSG00000172216 | 0.045856854 | 0.738238476 | 10.55473241 | 9.816493929 |
| 215596_s_at | LTN1 | ENSG00000198862 | 0.045856854 | 0.600695092 | 9.12314625 | 8.522451158 |
| 235306_at | GIMAP8 | ENSG00000171115 | 0.045948778 | 1.141523234 | 7.866981655 | 6.725458421 |
| 213746_s_at | FLNA | ENSG00000196924 | 0.045983205 | 0.670976446 | 10.55453024 | 9.883553792 |
| 200982_s_at | ANXA6 | ENSG00000197043 | 0.045983205 | 0.967756544 | 9.852305278 | 8.884548734 |
| 227029_at | FAM177A1 | ENSG00000151327 | 0.046043333 | 0.833008817 | 6.455041674 | 5.622032856 |
| 225695_at | SLC35F6 | ENSG00000213699 | 0.046225372 | 0.724610376 | 9.024413478 | 8.299803103 |
| 230263_s_at | DOCK5 | ENSG00000147459 | 0.046636223 | 0.984168534 | 5.975225342 | 4.991056808 |
| 219860_at | LY6G5C | ENSG00000204428 | 0.046660118 | 0.498836907 | 6.319773104 | 5.820936197 |
| 216620_s_at | ARHGEF10 | ENSG00000104728 | 0.046660118 | 1.005916383 | 8.01557698 | 7.009660598 |
| 209298_s_at | ITSN1 | ENSG00000205726 | 0.046711592 | 1.277202537 | 5.88081823 | 4.603615693 |
| 219383_at | PRR5L | ENSG00000135362 | 0.046711592 | 0.874143565 | 4.475309631 | 3.601166066 |
| 218204_s_at | FYCO1 | ENSG00000163820 | 0.046711592 | 0.475324889 | 7.841059718 | 7.36573483 |
| 212637_s_at | WWP1 | ENSG00000123124 | 0.046711592 | 0.922349431 | 7.411309223 | 6.488959792 |
| 200784_s_at | LRP1 | ENSG00000123384 | 0.046711592 | 0.835469409 | 10.71172473 | 9.876255321 |
| 202668_at | EFNB2 | ENSG00000125266 | 0.046711592 | 1.300618622 | 7.729295355 | 6.428676733 |
| 211926_s_at | MYH9 | ENSG00000100345 | 0.046850713 | 0.514027812 | 10.37283665 | 9.85880884 |
| 207181_s_at | CASP7 | ENSG00000165806 | 0.046856444 | 0.877318568 | 8.172928021 | 7.295609453 |
| 203940_s_at | VASH1 | ENSG00000071246 | 0.046903847 | 0.759053043 | 8.993567489 | 8.234514446 |
| 225046_at | LOC389831 | NA | 0.046903847 | 1.209299028 | 9.502744298 | 8.29344527 |
| 229699_at | LOC100129550 | NA | 0.046903847 | 0.787522797 | 7.252124657 | 6.46460186 |
| 1555997_s_at | IGFBP5 | ENSG00000115461 | 0.046903847 | 2.089302737 | 9.039541189 | 6.950238452 |
| 221858_at | TBC1D12 | ENSG00000108239 | 0.046903847 | 0.975449344 | 6.730088898 | 5.754639554 |
| 202123_s_at | ABL1 | ENSG00000097007 | 0.046903847 | 0.535932838 | 8.956464172 | 8.420531334 |
| 209189_at | FOS | ENSG00000170345 | 0.046903847 | 2.311395259 | 8.928569361 | 6.617174102 |
| 231697_s_at | VMP1 | ENSG00000062716 | 0.046903847 | 1.337676729 | 9.338248758 | 8.000572029 |
| 231823_s_at | SH3PXD2B | ENSG00000174705 | 0.046907121 | 1.342190597 | 8.749657487 | 7.40746689 |
| 226917_s_at | ANAPC4 | ENSG00000053900 | 0.046907121 | 0.7157989 | 9.420943217 | 8.705144317 |
| 213135_at | TIAM1 | ENSG00000156299 | 0.046959774 | 1.432495967 | 8.128730835 | 6.692234868 |
| 222793_at | DDX58 | ENSG00000107201 | 0.046959774 | 0.793030854 | 6.897065298 | 6.104034445 |
| 235059_at | RAB12 | ENSG00000206418 | 0.047160578 | 0.736169692 | 8.288455057 | 7.552285365 |
| 213364_s_at | SNX1 | ENSG00000028528 | 0.047357556 | 0.941321551 | 7.632325883 | 6.691004333 |
| 213194_at | ROBO1 | ENSG00000169855 | 0.047357556 | 1.202364784 | 8.299204466 | 7.096839682 |
| 223434_at | GBP3 | ENSG00000117226 | 0.047368037 | 1.767006321 | 8.155929476 | 6.388923155 |
| 201464_x_at | JUN | ENSG00000177606 | 0.047576751 | 1.411647741 | 9.379205572 | 7.967557831 |
| 228325_at | KIAA0146 | NA | 0.047576751 | 1.637115768 | 6.87876789 | 5.241652122 |
| 226639_at | SFT2D3 | ENSG00000173349 | 0.047769829 | 0.402970073 | 7.472242013 | 7.06927194 |
| 204204_at | SLC31A2 | ENSG00000136867 | 0.04778455 | 1.078146963 | 7.858561834 | 6.78041487 |
| 213659_at | ZNF75D | ENSG00000186376 | 0.04778455 | 0.665495605 | 7.859603651 | 7.194108047 |
| 219165_at | PDLIM2 | ENSG00000120913 | 0.047788444 | 0.537830248 | 8.659828829 | 8.12199858 |
| 202704_at | TOB1 | ENSG00000141232 | 0.047841124 | 1.157733162 | 7.864872509 | 6.707139347 |
| 201059_at | CTTN | ENSG00000085733 | 0.047841124 | 1.132979997 | 8.126828979 | 6.993848982 |
| 208626_s_at | VAT1 | ENSG00000108828 | 0.047842986 | 0.94552568 | 10.23981289 | 9.294287208 |
| 224285_at | GPR174 | ENSG00000147138 | 0.047886452 | 1.19745705 | 7.961716488 | 6.764259438 |
| 202746_at | ITM2A | ENSG00000078596 | 0.047974392 | 1.756296873 | 8.555659235 | 6.799362362 |
| 1557749_at | EHBP1L1 | ENSG00000173442 | 0.047976051 | 1.300818836 | 7.376097806 | 6.07527897 |
| 208671_at | SERINC1 | ENSG00000111897 | 0.047976051 | 0.972609476 | 8.578870199 | 7.606260723 |
| 235199_at | RNF125 | ENSG00000101695 | 0.048038754 | 1.064207451 | 7.065692015 | 6.001484564 |
| 236565_s_at | LARP6 | ENSG00000166173 | 0.048102844 | 0.684300387 | 5.230392066 | 4.546091679 |
| 204575_s_at | MMP19 | ENSG00000123342 | 0.048102844 | 0.946555962 | 7.578626866 | 6.632070904 |
| 221653_x_at | APOL2 | ENSG00000128335 | 0.048102844 | 0.588583201 | 10.11616798 | 9.527584778 |
| 224804_s_at | FAM219B | ENSG00000178761 | 0.048102844 | 0.651562841 | 8.895175581 | 8.24361274 |
| 202820_at | AHR | ENSG00000106546 | 0.048164884 | 1.191425195 | 8.433260381 | 7.241835186 |
| 204082_at | PBX3 | ENSG00000167081 | 0.048164884 | 0.63580907 | 8.576776457 | 7.940967387 |
| 218109_s_at | MFSD1 | ENSG00000118855 | 0.048304996 | 1.139673302 | 10.13940908 | 8.999735777 |
| 231897_at | PTGR1 | ENSG00000106853 | 0.048304996 | 1.154891244 | 7.789450843 | 6.634559598 |
| 205786_s_at | ITGAM | ENSG00000169896 | 0.048305577 | 1.137807787 | 8.121972126 | 6.984164339 |
| 203510_at | MET | ENSG00000105976 | 0.048305577 | 1.650209197 | 7.842578732 | 6.192369534 |
| 224896_s_at | TTL | ENSG00000114999 | 0.048305577 | 0.56036721 | 8.810442042 | 8.250074832 |
| 232645_at | LOC153684 | NA | 0.048305577 | 0.888956991 | 7.159878318 | 6.270921327 |
| 226576_at | ARHGAP26 | ENSG00000145819 | 0.048305577 | 0.822588192 | 6.112705611 | 5.290117419 |
| 219191_s_at | BIN2 | ENSG00000110934 | 0.048305577 | 1.097818549 | 9.34065692 | 8.242838371 |
| 214853_s_at | SHC1 | ENSG00000160691 | 0.048305577 | 0.580661886 | 10.02587289 | 9.445211 |
| 224358_s_at | MS4A7 | ENSG00000166927 | 0.048314327 | 2.066880998 | 8.506524406 | 6.439643408 |
| 209164_s_at | CYB561 | ENSG00000008283 | 0.04855986 | 0.862673302 | 8.334774098 | 7.472100797 |
| 222175_s_at | MED15 | ENSG00000099917 | 0.04855986 | 0.445190149 | 9.603400247 | 9.158210098 |
| 219469_at | DYNC2H1 | ENSG00000187240 | 0.048569792 | 0.659598577 | 6.555849742 | 5.896251165 |
| 226143_at | RAI1 | ENSG00000108557 | 0.048569792 | 1.208393363 | 8.541447862 | 7.333054499 |
| 212681_at | EPB41L3 | ENSG00000082397 | 0.048619009 | 1.347291945 | 8.071548526 | 6.724256581 |
| 215784_at | CD1E | ENSG00000158488 | 0.048710337 | 1.145368323 | 5.448260924 | 4.302892601 |
| 211161_s_at | COL3A1 | ENSG00000168542 | 0.048710337 | 2.029466261 | 11.58325522 | 9.553788957 |
| 209906_at | C3AR1 | ENSG00000171860 | 0.048810009 | 1.181583182 | 9.98497067 | 8.803387488 |
| 200625_s_at | CAP1 | ENSG00000131236 | 0.048951883 | 0.838593198 | 11.78722536 | 10.94863217 |
| 209550_at | NDN | ENSG00000182636 | 0.048952014 | 0.889205149 | 8.585921026 | 7.696715877 |
| 214039_s_at | LAPTM4B | ENSG00000104341 | 0.048974372 | 1.662825915 | 7.882223505 | 6.21939759 |
| 209356_x_at | EFEMP2 | ENSG00000172638 | 0.049063503 | 0.758705207 | 8.0078881 | 7.249182893 |
| 207276_at | CDR1 | ENSG00000184258 | 0.049177921 | 1.892594551 | 9.606611577 | 7.714017027 |
| 209955_s_at | FAP | ENSG00000078098 | 0.049177921 | 1.477706269 | 8.251596445 | 6.773890176 |
| 226844_at | MOB3B | ENSG00000120162 | 0.049203301 | 1.163764789 | 6.922109848 | 5.758345059 |
| 222468_at | KIAA0319L | ENSG00000142687 | 0.049203301 | 0.30117243 | 8.203036313 | 7.901863883 |
| 226056_at | ARHGAP31 | ENSG00000031081 | 0.049246985 | 0.787516152 | 8.433132327 | 7.645616175 |
| 226695_at | PRRX1 | ENSG00000116132 | 0.049316222 | 1.762734658 | 9.803780005 | 8.041045346 |
| 204844_at | ENPEP | ENSG00000138792 | 0.049441905 | 0.659515691 | 4.49151479 | 3.831999099 |
| 200612_s_at | AP2B1 | ENSG00000006125 | 0.049441905 | 0.595747097 | 8.555795692 | 7.960048594 |
| 200923_at | LGALS3BP | ENSG00000108679 | 0.049441905 | 0.921133422 | 10.07362245 | 9.152489033 |
| 208074_s_at | AP2S1 | ENSG00000042753 | 0.049593197 | 0.59579328 | 10.51238698 | 9.9165937 |
| 200897_s_at | PALLD | ENSG00000129116 | 0.049598626 | 1.601894471 | 9.526128512 | 7.924234041 |
| 209667_at | CES2 | ENSG00000172831 | 0.049598626 | 0.512790855 | 8.055153847 | 7.542362992 |
| 225785_at | REEP3 | ENSG00000165476 | 0.049648667 | 1.112993317 | 7.249649028 | 6.136655711 |
| 227265_at | FGL2 | ENSG00000127951 | 0.049648667 | 1.768021672 | 9.464203046 | 7.696181374 |
| 226679_at | SLC26A11 | ENSG00000181045 | 0.049648667 | 1.377482675 | 8.622059873 | 7.244577198 |
| 202441_at | ERLIN1 | ENSG00000107566 | 0.049690836 | 0.585414195 | 8.619248306 | 8.033834111 |
| 227758_at | RERG | ENSG00000134533 | 0.049697612 | 1.130937723 | 5.789853181 | 4.658915458 |
| 204036_at | LPAR1 | ENSG00000198121 | 0.049813564 | 1.444039399 | 6.674595378 | 5.230555979 |
| 225755_at | KLHDC8B | ENSG00000185909 | 0.049849481 | 0.85120088 | 8.00569222 | 7.15449134 |
| 209571_at | CIR1 | ENSG00000138433 | 0.049849481 | 0.376268664 | 8.299113302 | 7.922844638 |
| 210184_at | ITGAX | ENSG00000140678 | 0.049853347 | 1.112779508 | 8.872883821 | 7.760104313 |
| 217940_s_at | CARKD | ENSG00000213995 | 0.049853347 | 0.480686702 | 8.908728186 | 8.428041484 |
| 212993_at | NACC2 | ENSG00000148411 | 0.050114999 | 1.114936871 | 7.888031769 | 6.773067899 |
| 1562876_s_at | LOC541471 | NA | 0.050218071 | 0.682562423 | 3.603677506 | 2.921115083 |
| 242268_at | CELF2 | ENSG00000048740 | 0.050218071 | 1.553668129 | 7.210053313 | 5.656385183 |
| 213125_at | OLFML2B | ENSG00000162745 | 0.050218071 | 0.787465426 | 8.741575545 | 7.654110119 |
| 221257_x_at | FBXO38 | ENSG00000145868 | 0.050300966 | 0.597625209 | 7.698194987 | 7.100569778 |
| 236782_at | SAMD3 | ENSG00000164483 | 0.050399558 | 1.072740707 | 7.590990221 | 6.518249514 |
| 1554690_a_at | TACC1 | ENSG00000147526 | 0.050505576 | 1.079368013 | 9.700334844 | 8.620966831 |
| 203431_s_at | ARHGAP32 | ENSG00000134909 | 0.050518588 | 1.238339866 | 6.793820795 | 5.555480928 |
| 226711_at | FOXN2 | ENSG00000170802 | 0.050635583 | 0.937101457 | 9.838675692 | 8.901574235 |
| 203562_at | FEZ1 | ENSG00000149557 | 0.050635583 | 1.424636375 | 7.169090802 | 5.744454428 |
| 201508_at | IGFBP4 | ENSG00000141753 | 0.050635583 | 1.218153348 | 10.95048091 | 9.732327564 |
| 209933_s_at | CD300A | ENSG00000167851 | 0.050635583 | 1.063950828 | 8.653599864 | 7.589649035 |
| 226751_at | CNRIP1 | ENSG00000119868 | 0.05098315 | 1.046366586 | 7.446206604 | 6.399840018 |
| 203303_at | DYNLT3 | ENSG00000165169 | 0.05098315 | 0.838978072 | 9.479702344 | 8.640724272 |
| 1556698_a_at | GPRIN3 | ENSG00000185477 | 0.05098315 | 1.215337617 | 6.331782061 | 5.116444444 |
| 223454_at | CXCL16 | ENSG00000161921 | 0.05098315 | 0.987094685 | 9.755064685 | 8.76797 |
| 202032_s_at | MAN2A2 | ENSG00000196547 | 0.05098315 | 0.885891887 | 9.56468737 | 8.678795483 |
| 205779_at | RAMP2 | ENSG00000131477 | 0.051156378 | 0.933822492 | 8.483654672 | 7.54983218 |
| 209110_s_at | RGL2 | ENSG00000237441 | 0.051156378 | 0.662542944 | 8.743821756 | 8.081278812 |
| 209935_at | ATP2C1 | ENSG00000017260 | 0.051156378 | 0.657015693 | 6.368275466 | 5.711259774 |
| 214453_s_at | IFI44 | ENSG00000137965 | 0.051156378 | 1.633185311 | 9.486683614 | 7.853498302 |
| 201063_at | RCN1 | ENSG00000049449 | 0.051233232 | 0.702809737 | 9.216222535 | 8.513412798 |
| 213222_at | PLCB1 | ENSG00000182621 | 0.051361434 | 0.700475436 | 5.535979887 | 3.835504451 |
| 201069_at | MMP2 | ENSG00000087245 | 0.051361434 | 1.691353818 | 10.01928343 | 8.327929615 |
| 200661_at | CTSA | ENSG00000064601 | 0.051361434 | 0.720607027 | 10.64337094 | 9.922763911 |
| 213258_at | TFPI | ENSG00000003436 | 0.051378943 | 1.419437821 | 8.029849535 | 6.610411714 |
| 225414_at | RNF149 | ENSG00000163162 | 0.05142224 | 0.804672421 | 9.112269744 | 8.307597324 |
| 219397_at | COQ10B | ENSG00000115520 | 0.05142224 | 0.654479835 | 7.359951703 | 6.705471868 |
| 222127_s_at | EXOC1 | ENSG00000090989 | 0.051602523 | 0.754363501 | 9.746303928 | 8.991940427 |
| 223220_s_at | PARP9 | ENSG00000138496 | 0.051602523 | 0.979949321 | 9.260981734 | 8.281032413 |
| 213422_s_at | MXRA8 | ENSG00000162576 | 0.051602523 | 1.242243727 | 8.999253285 | 7.757009558 |
| 203153_at | IFIT1 | ENSG00000185745 | 0.051753444 | 1.528926589 | 7.71997217 | 6.191045582 |
| 202100_at | RALB | ENSG00000144118 | 0.051843475 | 0.671402473 | 8.880755781 | 8.209353307 |
| 224895_at | YAP1 | ENSG00000137693 | 0.051946417 | 1.490945911 | 7.474062759 | 5.983116848 |
| 201368_at | ZFP36L2 | ENSG00000152518 | 0.052319048 | 0.964711787 | 10.56868804 | 9.603976254 |
| 212027_at | RBM25 | ENSG00000119707 | 0.052384081 | 0.678190641 | 9.494823888 | 8.816633247 |
| 221942_s_at | GUCY1A3 | ENSG00000164116 | 0.052439589 | 1.367770663 | 7.987538556 | 6.619767893 |
| 213800_at | CFH | ENSG00000000971 | 0.052439589 | 1.232285055 | 7.757236819 | 6.524950764 |
| 1554999_at | RASGEF1B | ENSG00000138670 | 0.052613981 | 1.417397531 | 6.341497404 | 4.924099873 |
| 218606_at | ZDHHC7 | ENSG00000153786 | 0.052736978 | 0.390537911 | 8.928963607 | 8.538425696 |
| 204083_s_at | TPM2 | ENSG00000198467 | 0.052793888 | 1.005641012 | 10.06504289 | 9.059401879 |
| 1553955_at | PPP1R21 | ENSG00000162869 | 0.052932924 | 0.83883015 | 8.379910468 | 7.541080318 |
| 228083_at | CACNA2D4 | ENSG00000151062 | 0.052932924 | 0.833099044 | 7.25294855 | 6.419849507 |
| 227304_at | SMCR8 | ENSG00000176994 | 0.052932924 | 0.799991418 | 8.377826966 | 7.577835548 |
| 225710_at | GNB4 | ENSG00000114450 | 0.052932924 | 1.087540407 | 8.393034502 | 7.305494096 |
| 204154_at | CDO1 | ENSG00000129596 | 0.052932924 | 1.192918606 | 5.120926441 | 3.928007836 |
| 228318_s_at | CRIPAK | ENSG00000179979 | 0.05300806 | 0.531106343 | 6.682490506 | 6.151384164 |
| 227013_at | LATS2 | ENSG00000150457 | 0.05300806 | 1.10106835 | 7.79320732 | 6.692138971 |
| 1558692_at | C1orf85 | ENSG00000198715 | 0.053110207 | 0.572438959 | 5.400511953 | 4.828072994 |
| 213238_at | ATP10D | ENSG00000145246 | 0.053110207 | 1.051038069 | 8.615730447 | 7.564692379 |
| 201850_at | CAPG | ENSG00000042493 | 0.053135419 | 0.809383904 | 10.75410257 | 9.944718664 |
| 209717_at | EVI5 | ENSG00000067208 | 0.053140372 | 0.711449575 | 7.081763345 | 6.37031377 |
| 227388_at | TUSC1 | ENSG00000198680 | 0.053261035 | 1.564367321 | 8.139272536 | 6.574905215 |
| 200771_at | LAMC1 | ENSG00000135862 | 0.053546476 | 0.885804076 | 8.418291529 | 7.532487453 |
| 229055_at | GPR68 | ENSG00000119714 | 0.053546476 | 0.837700456 | 9.226090778 | 8.388390321 |
| 200927_s_at | RAB14 | ENSG00000119396 | 0.053623502 | 0.522640122 | 8.220465934 | 7.697825812 |
| 225447_at | GPD2 | ENSG00000115159 | 0.053623502 | 0.967759668 | 8.485215298 | 7.51745563 |
| 225525_at | KIAA1671 | ENSG00000197077 | 0.053800235 | 1.446748208 | 7.312947558 | 5.866199349 |
| 209649_at | STAM2 | ENSG00000115145 | 0.053803885 | 0.757498312 | 7.629711981 | 6.872213668 |
| 219492_at | CHIC2 | ENSG00000109220 | 0.054091605 | 0.67290252 | 8.38646188 | 7.71355936 |
| 1560060_s_at | VPS37C | ENSG00000167987 | 0.054133103 | 0.55757033 | 8.463033953 | 7.905463623 |
| 217947_at | CMTM6 | ENSG00000091317 | 0.054171637 | 0.70230484 | 10.47748323 | 9.775178387 |
| 219078_at | GPATCH2 | ENSG00000092978 | 0.054197198 | 0.95185617 | 7.045735811 | 6.093879641 |
| 203854_at | CFI | ENSG00000205403 | 0.05429229 | 1.205739531 | 6.704059047 | 5.498319516 |
| 209637_s_at | RGS12 | ENSG00000159788 | 0.054327713 | 0.502100289 | 7.515977537 | 7.013877248 |
| 202252_at | RAB13 | ENSG00000143545 | 0.05435009 | 0.913757531 | 9.579585924 | 8.665828393 |
| 227373_at | ATXN1L | ENSG00000224470 | 0.05435009 | 0.759716522 | 8.404084446 | 7.644367924 |
| 207469_s_at | PIR | ENSG00000087842 | 0.054460455 | 0.663778954 | 6.265059241 | 5.601280287 |
| 212543_at | AIM1 | ENSG00000112297 | 0.054463686 | 0.788119117 | 9.682280231 | 8.894161115 |
| 208983_s_at | PECAM1 | NA | 0.054523626 | 1.305077039 | 10.69253504 | 9.387459 |
| 226438_at | SNTB1 | ENSG00000172164 | 0.054649274 | 0.809609573 | 6.953942186 | 6.144332613 |
| 201567_s_at | GOLGA4 | ENSG00000144674 | 0.054736968 | 0.544061301 | 8.834091632 | 8.290030331 |
| 204520_x_at | BRD1 | ENSG00000100425 | 0.05484599 | 0.438369852 | 9.008739811 | 8.570369959 |
| 217828_at | SLTM | ENSG00000137776 | 0.05484599 | 0.830702603 | 8.864809369 | 8.034106766 |
| 224689_at | MANBAL | ENSG00000101363 | 0.05484599 | 0.340215626 | 9.457076055 | 9.11686043 |
| 219157_at | KLHL2 | ENSG00000109466 | 0.054892546 | 0.636752761 | 8.271660585 | 7.634907824 |
| 232024_at | GIMAP2 | ENSG00000106560 | 0.055019604 | 1.162132159 | 7.593742729 | 6.431610571 |
| 219087_at | ASPN | ENSG00000106819 | 0.05508081 | 1.844431057 | 7.912112086 | 6.06768103 |
| 213010_at | PRKCDBP | ENSG00000170955 | 0.05508081 | 0.666737424 | 7.157466 | 6.490728576 |
| 219570_at | KIF168 | ENSG00000089177 | 0.05508081 | 0.939524718 | 6.792995577 | 5.853470859 |
| 214066_x_at | NPR2 | ENSG00000159899 | 0.05508081 | 0.499560523 | 7.871514788 | 7.371954265 |
| 228372_at | C10orf128 | ENSG00000204161 | 0.05508081 | 1.228253425 | 8.331788019 | 7.103534595 |
| 230276_at | FAM49A | ENSG00000197872 | 0.05508081 | 1.286488945 | 6.31293966 | 5.026450715 |
| 203002_at | AMOTL2 | ENSG00000114019 | 0.05508081 | 1.381170498 | 7.607053309 | 6.225882812 |
| 225688_s_at | PHLDB2 | ENSG00000144824 | 0.05508081 | 1.642110715 | 7.415101954 | 5.773291239 |
| 1553678_a_at | ITGB1 | ENSG00000150093 | 0.05508081 | 0.762328098 | 9.60484688 | 8.842518782 |
| 203476_at | TPBG | ENSG00000146242 | 0.05508081 | 1.402870201 | 8.397859019 | 6.994988818 |
| 201180_s_at | GNAI3 | ENSG00000065135 | 0.05508081 | 0.716484855 | 9.753542862 | 9.037058006 |
| 220141_at | C11orf63 | ENSG00000109944 | 0.05508081 | 0.530289066 | 5.631726383 | 5.101437317 |
| 227923_at | SHANK3 | ENSG00000251322 | 0.05508081 | 1.175626141 | 7.738817565 | 6.563191424 |
| 235334_at | ST6GALNAC3 | ENSG00000184005 | 0.05508081 | 0.832570926 | 5.744060873 | 4.911489947 |
| 235411_at | PGBD1 | ENSG00000137338 | 0.05508081 | 0.454906286 | 5.741778418 | 5.286872132 |
| 216689_x_at | ARHGAP1 | ENSG00000175220 | 0.055086404 | 0.521010788 | 9.747525869 | 9.226515081 |
| 202932_at | YES1 | ENSG00000176105 | 0.055086404 | 1.212404455 | 6.259729591 | 5.047325136 |
| 212293_at | HIPK1 | ENSG00000163349 | 0.055094773 | 0.690816647 | 7.914422373 | 7.223605727 |
| 225618_at | ARHGAP27 | ENSG00000159314 | 0.055334195 | 0.473020908 | 7.935836992 | 7.462816084 |
| 201242_s_at | ATP1B1 | ENSG00000143153 | 0.055334195 | 1.283149532 | 7.126474102 | 5.84332457 |
| 203583_at | UNC50 | ENSG00000115446 | 0.055334195 | 0.452783056 | 8.989706349 | 8.536923292 |
| 1557938_s_at | PTRF | ENSG00000177469 | 0.055334195 | 0.894352142 | 8.261399661 | 7.367047519 |
| 242321_at | PTPN14 | ENSG00000152104 | 0.055341667 | 1.326981685 | 5.787995241 | 4.461013556 |
| 212950_at | GPR116 | ENSG00000069122 | 0.055341667 | 1.466287536 | 6.762014415 | 5.295726879 |
| 205638_at | AOAH | ENSG00000136250 | 0.055341667 | 0.654520054 | 9.612122833 | 8.957602778 |
| 223209_s_at | VIMP | ENSG00000131871 | 0.055341667 | 0.83813242 | 9.523564232 | 8.685431812 |
| 208131_s_at | PTGIS | ENSG00000124212 | 0.055389281 | 1.321200027 | 7.490939927 | 6.1697399 |
| 227833_s_at | MBD6 | ENSG00000166987 | 0.055396564 | 0.751922649 | 10.51819974 | 9.766277089 |
| 208991_at | STAT3 | ENSG00000168610 | 0.055396564 | 0.946587005 | 9.530424681 | 8.583837676 |
| 216361_s_at | KAT6A | ENSG00000083168 | 0.055396564 | 0.651745657 | 7.927871972 | 7.276126315 |
| 1554106_at | NBEAL1 | ENSG00000144426 | 0.055396564 | 0.661916069 | 7.462248226 | 6.800332157 |
| 209357_at | CITED2 | ENSG00000164442 | 0.055396564 | 1.232382537 | 9.303494571 | 8.071112033 |
| 53720_at | C19orf66 | ENSG00000130813 | 0.055431756 | 0.967375092 | 9.679981688 | 8.712606595 |
| 228964_at | PRDM1 | ENSG00000057657 | 0.055431756 | 1.955052921 | 9.250667771 | 7.295614851 |
| 228624_at | TMEM144 | ENSG00000164124 | 0.055512399 | 0.888115651 | 6.82051307 | 5.932397419 |
| 225626_at | PAG1 | ENSG00000076641 | 0.055625052 | 1.648320718 | 9.726492347 | 8.07817163 |
| 225885_at | EEA1 | ENSG00000102189 | 0.055625052 | 0.727821543 | 7.739962682 | 7.012141139 |
| 236172_at | LTB4R | ENSG00000213903 | 0.055654381 | 1.08404573 | 7.38748053 | 6.3034348 |
| 201953_at | CIB1 | ENSG00000185043 | 0.055677119 | 0.709254173 | 9.414667247 | 8.705413075 |
| 213069_at | HEG1 | ENSG00000173706 | 0.055677119 | 1.058297106 | 9.011754037 | 7.953456931 |
| 226885_at | RNF217 | ENSG00000146373 | 0.055687877 | 1.507951738 | 5.917910835 | 4.409959097 |
| 203038_at | PTPRK | ENSG00000152894 | 0.055687877 | 1.549533171 | 8.029702811 | 6.48016964 |
| 240703_s_at | HERC1 | ENSG00000103657 | 0.055700396 | 0.961787237 | 6.445277876 | 5.48349064 |
| 221641_s_at | ACOT9 | ENSG00000123130 | 0.055791741 | 0.592409727 | 9.037040731 | 8.444631004 |
| 210845_s_at | PLAUR | ENSG00000011422 | 0.055813214 | 0.996379681 | 8.86607143 | 7.869691749 |
| 212097_at | CAV1 | ENSG00000105974 | 0.055813214 | 1.72714647 | 8.888796375 | 7.161649906 |
| 243198_at | TEX9 | ENSG00000151575 | 0.055813214 | 1.558466496 | 6.115528068 | 4.557061571 |
| 204122_at | TYROBP | ENSG00000011600 | 0.055813214 | 1.153345531 | 11.48995674 | 10.33661121 |
| 226343_at | DPP8 | ENSG00000074603 | 0.055813214 | 0.667567668 | 8.292781672 | 7.625214004 |
| 205715_at | BST1 | ENSG00000109743 | 0.056095876 | 0.344955791 | 7.18626587 | 6.841310079 |
| 211980_at | COL4A1 | ENSG00000187498 | 0.056095876 | 1.223646645 | 10.90017588 | 9.676529238 |
| 213572_s_at | SERPINB1 | ENSG00000021355 | 0.056095876 | 0.852583509 | 9.447180216 | 8.594596707 |
| 209047_at | AQP1 | ENSG00000240583 | 0.056095876 | 1.711689396 | 8.767180781 | 7.055491386 |
| 214077_x_at | MEIS3P1 | ENSG00000179277 | 0.056095876 | 0.924586393 | 8.655448343 | 7.730861949 |
| 202946_s_at | BTBD3 | ENSG00000132640 | 0.056281665 | 1.008164542 | 5.025902254 | 4.017737712 |
| 242953_at | ZNF234 | ENSG00000263002 | 0.056315736 | 0.863642317 | 6.410969189 | 5.547326872 |
| 218380_at | LOC728392 | NA | 0.056642523 | 0.659862844 | 8.050295783 | 7.390432938 |
| 215000_s_at | FEZ2 | ENSG00000171055 | 0.056642523 | 0.895521867 | 9.749836768 | 8.854314901 |
| 229238_at | C17orf97 | ENSG00000187624 | 0.056642523 | 0.793626222 | 7.026857654 | 6.233231432 |
| 219700_at | PLXDC1 | ENSG00000161381 | 0.056642523 | 1.303729295 | 7.282377392 | 5.978648097 |
| 202432_at | PPP3CB | ENSG00000107758 | 0.056855469 | 0.594665801 | 8.654289616 | 8.059623814 |
| 1558711_at | FAM13A-AS1 | ENSG00000248019 | 0.05685832 | 0.663485851 | 6.443455817 | 5.779969966 |
| 202304_at | FNDC3A | ENSG00000102531 | 0.05685832 | 0.958277594 | 8.951196819 | 7.992919225 |
| 213429_at | BICC1 | ENSG00000122870 | 0.05685832 | 1.455435599 | 5.911436667 | 4.456001068 |
| 203388_at | ARRB2 | ENSG00000141480 | 0.05685832 | 0.790037034 | 8.830550615 | 8.040513531 |
| 205140_at | FPGT | ENSG00000254685 | 0.056862868 | 0.746130122 | 7.424392378 | 6.678262256 |
| 226594_at | ENTPD5 | ENSG00000187097 | 0.056942273 | 0.5571412 | 7.32471655 | 6.767575351 |
| 219506_at | C1orf54 | ENSG00000118292 | 0.056942273 | 1.14432295 | 10.50282075 | 9.358497804 |
| 209230_s_at | NUPR1 | ENSG00000176046 | 0.056996975 | 1.108100553 | 11.12843917 | 10.02033862 |
| 205498_at | GHR | ENSG00000112964 | 0.057095384 | 1.399155512 | 5.181886139 | 3.782730626 |

| probeset | Chromosome | chrom_band | gene_description |
|---|---|---|---|
| 228754_at | 3 | p25.1 | solute carrier family 6 [neurotransmitter transporter, taurine), |
| | | | member 6 [Source: HGNC Symbol; Acc: 11052] |
| 238327_at | 22 | q13.33 | outer dense fiber of sperm tails 3B [Source: HGNC |
| | | | Symbol, Acc: 34388] |
| 208683_at | 1 | q41 | calpain 2, (m/ll) large subunit [Source: HGNC Symbol; Acc: 1479] |
| 218648_at | 15 | q26.1 | CREB regulated transcription coactivator 3 [Source: HGNC |
| | | | Symbol; Acc: 26148] |
| 225146_at | 9 | p13.3 | family with sequence similarity 219, member A [Source: HGNC |
| | | | Symbol; Acc: 19920] |
| 218589_at | 13 | q14.2 | lysophosphatidic acid receptor 6 [Source: HGNC Symbol; Acc: 15520] |
| 217762_s_at | 18 | p11.22 | RAB31, member RAS oncogene family [Source: HGNC |
| | | | Symbol; Acc: 9771] |
| 201506_at | 5 | q31.1 | transforming growth factor, beta-induced, 65 kDa [Source: HGNC |
| | | | Symbol; Acc: 11771] |
| 224862_at | 9 | q21.2 | guanine nucleotide binding protein (G protein), q polypeptide |
| | | | [Source: HGNC Symbol; Acc: 4390] |
| 201425_at | 12 | q24.12 | aldehyde dehydrogenase 2 family (mitochondrial) [Source: HGNC |
| | | | Symbol; Acc: 404] |
| 1555851_s_at | 19 | q13.33 | selenoprotein W, 1 [Source: HGNC Symbol; Acc: 10752] |
| 205241_at | 22 | q13.33 | SCO2 cytochrome c oxidase assembly protein [Source: HGNC |
| | | | Symbol; Acc: 10604] |
| 218552_at | 1 | p32.3 | enoyl CoA hydratase domain containing 2 [Source: HGNC |
| | | | Symbol; Acc: 23408] |
| 55662_at | 10 | q24.32 | chromsome 10 open reading frame 76 [Source: HGNC |
| | | | Symbol; Acc: 25788] |
| 204773_at | 9 | p13.3 | interleukin 11 receptor, alpha [Source: HGNC Symbol; Acc: 5967] |
| 202600_s_at | 21 | q21.1 | nuclear receptor interacting protein 1 [Source: HGNC |
| | | | Symbol; Acc: 8001] |
| 221802_s_at | 10 | q25.3 | KIAA1598 [Source: HGNC Symbol; Acc: 29319] |
| 226000_at | 1 | p13.2 | CTTNBP2 N-terminal like [Source: HGNC Symbol; Acc: 25330] |
| 222484_s_at | 5 | q31.1 | chemokine (C-X-C motif) ligand 14 [Source: HGNC |
| | | | Symbol; Acc: 10640] |
| 201012_at | 9 | q21.13 | annexin A1 [Source: HGNC Symbol; Acc: 533] |
| 218854_at | 6 | q22.1 | dermatan sulfate epimerase [Source: HGNC Symbol; Acc: 21144] |
| 214040_s_at | 9 | q33.2 | gelsolin [Source: HGNC Symbol; Acc: 4620] |
| 201302_at | 2 | p13.3 | annexin A4 [Source: HGNC Symbol; Acc: 542] |
| 208923_at | 15 | q11.2 | cytoplasmic FMR1 interacting protein 1 [Source: HGNC |
| | | | Symbol; Acc: 13759] |
| 224414_s_at | 5 | p13.1 | caspase recruitment domain family, member 6 [Source: HGNC |
| | | | Symbol; Acc: 16394] |
| 205945_at | 1 | q21.3 | interleukin 6 receptor [Source: HGNC Symbol; Acc: 6019] |
| 200765_x_at | 5 | q31.2 | catenin (cadherin-associated protein), alpha 1, 102 kDa |
| | | | [Source: HGNC Symbol; Acc: 2509] |
| 230325_at | NA | NA | NA |
| 223228_at | 22 | q13.31 | leucine zipper, down-regulated in cancer 1-like [Source: HGNC |
| | | | Symbol; Acc: 13343] |
| 225842_at | 12 | q21.2 | pleckstrin homology-like domain, family A, member 1 |
| | | | [Source: HGNC Symbol; Acc: 8933] |
| 201348_at | 5 | q33.1 | glutathione peroxidase 3 (plasma) [Source: HGNC Symbol; Acc: 4555] |
| 219885_at | 17 | q12 | schlafen family member 12 [Source: HGNC Symbol; Acc: 25500] |
| 212830_at | 9 | q33.2 | multiple EGF-like domains 9 [Source: HGNC Symbol; Acc: 3234] |
| 231463_at | 17 | q21.31 | cyclin N-terminal domain containing 1 [Source: HGNC |
| | | | Symbol; Acc: 26847] |
| 202687_s_at | 3 | q26.31 | tumor necrosis factor (ligand) superfamily, member 10 |
| | | | [Source: HGNC Symbol; Acc: 11925] |
| 217731_s_at | 13 | q14.2 | integral membrane protein 2B [Source: HGNC Symbol; Acc: 6174] |
| 218694_at | X | q22.1 | armadillo repeat containing, X-linked 1 [Source: HGNC |
| | | | Symbol; Acc: 18073] |
| 1558844_at | NA | NA | NA |
| 202944_at | 22 | q13.2 | N-acetylgalactosaminidase, alpha-[Source: HGNC Symbol; Acc: 7631] |
| 212522_at | 15 | q25.3 | phosphodiesterase 8A [Source: HGNC Symbol; Acc: 8793] |
| 226247_at | 10 | q26.13 | pleckstrin homology domain containing, family A (phophoinositide |
| | | | binding specific) member 1 [Source: HGNC Symbol; Acc: 14335] |
| 243423_at | 5 | q33.1 | TNFAIP3 interacting protein 1 [Source: HGNC Symbol; Acc: 16903] |
| 229074_at | 15 | q15.1 | EH-domain containing 4 [Source: HGNC Symbol; Acc: 3245] |
| 1559507_at | NA | NA | NA |
| 222154_s_at | 2 | q33.1 | spermatogenesis associated, serine-rich 2-like [Source: HGNC |
| | | | Symbol; Acc: 24574] |
| 200710_at | 17 | p13.1 | acyl-CoA dehydrogenase, very long chain [Source: HGNC |
| | | | Symbol; Acc: 92] |
| 228791_at | NA | NA | NA |
| 208634_s_at | 1 | p34.3 | microtubule-actin crosslinking factor 1 [Source: HGNC |
| | | | Symbol; Acc: 13664] |
| 242487_at | 1 | p32.3 | coiled-coil and C2 domain 1B [Source: HGNC |
| | | | Symbol; Acc: 29386] |
| 218559_s_at | 20 | q12 | v-maf avian musculoaponeurotic fibrosarcoma oncogene homolog |
| | | | B [Source: HGNC Symbol; Acc: 6408] |
| 201360_at | 20 | p11.21 | cystatin C [Source: HGNC Symbol; Acc: 2475] |
| | | | extracellular matrix protein 2, female organ and adipocyte specific |
| 206101_at | 9 | q22.31 | [Source: HGNC Symbol; Acc: 3154] |
| 218004_at | 1 | p35.1 | BSD domain containing 1 [Source: HGNC Symbol; Acc: 25501] |
| 36129_at | 17 | p13.3 | small G protein signaling modulator 2 [Source: HGNC |
| | | | Symbol; Acc: 29026] |
| 227889_at | 16 | q12.2 | lysophosphatidylcholine acyltransferase 2 [Source: HGNC |
| | | | Symbol; Acc: 26032] |
| 229378_at | 10 | q21.3 | storkhead box 1 [Source: HGNC Symbol; Acc: 23508] |
| 218043_s_at | 3 | p24.1 | 5-azactidine induced 2 [Source: HGNC Symbol; Acc: 24002] |
| 218066_at | 5 | p15.33 | solute carrier family 12 (potassium/chloride transporters), member |
| | | | 7 [Source: HGNC Symbol; Acc: 10915] |
| 58780_s_at | 14 | q11.2 | Rho guanine nucleotide exchange factor (GEF) 40 [Source: HGNC |
| | | | Symbol; Acc: 25516] |
| 200660_at | 1 | q21.3 | S100 calcium binding protein A11 [Source: HGNC |
| | | | Symbol; Acc: 10488] |
| 207965_at | 10 | q22.1 | neurogenin 3 [Source: HGNC Symbol; Acc: 13806] |
| 212907_at | 1 | q32.3 | solute carrier family 30 (zinc transporter), member 1 [Source: HGNC |
| | | | Symbol; Acc: 11012] |
| 208999_at | 5 | q31.1 | septin 8 [Source: HGNC Symbol; Acc: 16511] |
| 208949_s_at | 14 | q22.3 | lectin, galactoside-binding, soluble, 3 [Source: HGNC |
| | | | Symbol; Acc: 6563] |
| 218311_at | 2 | p22.1 | mitogen-activated protein kinase kinase kinase kinase 3 |
| | | | [Source: HGNC Symbol; Acc: 6865] |
| 203518_at | 1 | q42.3 | lysosomal trafficking regulator [Source: HGNC Symbol; Acc: 1968] |
| 1552349_a_at | 16 | p13.3 | protease, serine, 33 [Source: HGNC Symbol; Acc: 30405] |
| 1566134_at | 16 | p13.2 | calcium regulated heat stable protein 1, 24 kDa [Source: HGNC |
| | | | Symbol; Acc: 17150] |
| 204137_at | 1 | q42.3 | G protein-coupled receptor 137B [Source: HGNC |
| | | | Symbol; Acc: 11862] |
| 222217_s_at | 1 | q21.3 | solute carrier family 27 (fatty acid transporter), member 3 |
| | | | [Source: HGNC Symbol; Acc: 10997] |
| 201505_at | 7 | q31.1 | laminin, beta 1 [Source: HGNC Symbol; Acc: 6486] |
| 206602_s_at | 2 | q31.1 | homeobox D3 [Source: HGNC Symbol; Acc: 5137] |
| 217728_at | 1 | q21.3 | S100 calcium binding protein A6 [Source: HGNC Symbol; Acc: 10496] |
| 225483_at | 11 | q25 | vacoular protein sorting 26 homolog B (S. pombe) [Source: HGNC |
| | | | Symbol; Acc: 28119] |
| 202686_s_at | 19 | q13.2 | AXL receptor tyrosine kinase [Source: HGNC Symbol; Acc: 905] |
| 236374_at | 5 | q23.2 | cortexin 3 [Source: HGNC Symbol; Acc: 31110] |
| 227276_at | 10 | p12.31 | plexin domain containing 2 [Source: HGNC Symbol; Acc: 21013] |
| 228185_at | 10 | p11.1 | zinc finger protein 25 [Source: HGNC Symbol; Acc: 13043] |
| 217892_s_at | 12 | q13.12 | LIM domain and actin binding 1 [Source: HGNC Symbol; Acc: 24636] |
| 202727_s_at | 6 | q23.3 | interferon gamma receptor 1 [Source: HGNC Symbol; Acc: 5439] |
| 1560963_a_at | NA | NA | NA |
| 1553837_at | 12 | q24.33 | phosphoglycerate mutase family member 5 [Source: HGNC |
| | | | Symbol; Acc: 28763] |
| 1554763_at | X | q21.1 | ubiquitin-conjugating enzyme E2D N-terminal like (pseudogene) |
| | | | [Source: HGNC Symbol; Acc: 28656] |
| 212112_s_at | 1 | p35.3 | syntaxin 12 [Source: HGNC Symbol; Acc: 11430] |
| 203789_s_at | 7 | q21.11 | sema domain, immunoglobulin domain (lg), short basic domain, |
| | | | secreted, (semaphorin), 3C [Source: HGNC Symbol; Acc: 10725] |
| 200677_at | 21 | q22.3 | pituitary tumor-transforming 1 interacting protein [Source: HGNC |
| | | | Symbol; Acc: 13524] |
| 222876_s_at | 17 | q11.2 | ArfGAP with dual PH domains 2 [Source: HGNC Symbol; Acc: 16487] |
| 210145_at | 1 | q31.1 | phospholipase A2, group IVA (cytosolic, calcium-dependent) |
| | | | [Source: HGNC Symbol; Acc: 9035] |
| 208109_s_at | NA | NA | NA |
| 209651_at | 16 | p11.2 | transforming growth factor beta 1 induced transcript 1 |
| | | | [Source: HGNC Symbol; Acc: 11767] |
| 212698_s_at | 2 | q13 | septin 10 [Source: HGNC Symbol; Acc: 14349] |
| 212526_at | 13 | q13.3 | spastic paraplegia 20 (Troyer syndrome) [Source: HGNC |
| | | | Symbol; Acc: 18514] |
| 209684_at | 20 | p11.23 | Ras and Rab interactor 2 [Source: HGNC Symbol; Acc: 18750] |
| 223204_at | 4 | q32.1 | family with sequence similarity 198, member B [Source: HGNC |
| | | | Symbol; Acc: 25312] |
| 200673_at | 2 | p24.1 | lysosomal protein transmembrane 4 alpha [Source: HGNC |
| | | | Symbol; Acc: 6924] |
| 219840_s_at | 14 | q32.13 | T-cell leukemia/lymphoma 6 (non-protein coding) [Source: HGNC |
| | | | Symbol; Acc: 13463] |
| 222896_at | 19 | p13.11 | transmembrane protein 38A [Source: HGNC Symbol; Acc: 28462] |
| 205688_at | 16 | p13.3 | transcription factor AP-4 (activating enhancer binding protein 4) |
| | | | [Source: HGNC Symbol; Acc: 11745] |
| 225384_at | 1 | p31.3 | dedicator of cytokinesis 7 [Source: HGNC Symbol; Acc: 19190] |
| 1557021_s_at | NA | NA | NA |
| 209210_s_at | 14 | q22.1 | fermitin family member 2 [Source: HGNC Symbol; Acc: 15767] |
| 201798_s_at | 10 | q23.33 | myoferlin [Source: HGNC Symbol; Acc: 3656] |
| 225949_at | 8 | q24.3 | nuclear receptor binding protein 2 [Source: HGNC |
| | | | Symbol; Acc: 19339] |
| 208924_at | 1 | p32.3 | ring finger protein 11 [Source: HGNC Symbol; Acc: 10056] |
| 209004_s_at | 4 | p15.32 | F-box and leucine-rich repeat protein 5 [Source: HGNC |
| | | | Symbol; Acc: 13602] |
| 237880_at | NA | NA | NA |
| 226743_at | 17 | q12 | schlafen family member 11 [Source: HGNC Symbol; Acc: 26633] |
| 228573_at | 4 | q21.21 | anthrax toxin receptor 2 [Source: HGNC Symbol; Acc: 21732] |
| 222065_s_at | 17 | p11.2 | flightless I homolog (Drosophila) [Source: HGNC Symbol; Acc: 3750] |
| 210450_at | 14 | q32.33 | immunoglobulin heavy variable 5-78 (pseudogene) [Source: HGNC |
| | | | Symbol; Acc: 5660] |
| 212989_at | 10 | q11.23 | sphingomyelin synthase 1 [Source: HGNC Symbol; Acc: 29799] |
| 202228_s_at | 15 | q24.1 | neuroplastin [Source: HGNC Symbol; Acc: 17867] |
| 227761_at | 15 | q21.2 | myosin VA (heavy chain 12, myoxin) [Source: HGNC |
| | | | Symbol; Acc: 7602] |
| 202133_at | 3 | q25.1 | WW domain containing transcription regulator 1 [Source: HGNC |
| | | | Symbol; Acc: 24042] |
| 209960_at | 7 | q21.11 | hepatocyte growth factor (hepapoietin A; scatter factor) |
| | | | [Source: HGNC Symbol; Acc: 4893] |
| 212203_x_at | 11 | p15.5 | interferon induced transmembrane protein 3 [Source: HGNC |
| | | | Symbol; Acc: 5414] |
| 212158_at | 8 | q22.1 | syndecan 2 [Source: HGNC Symbol; Acc: 10659] |
| 224797_at | 5 | q14.3 | arrestin domain containing 3 [Source: HGNC Symbol; Acc: 29263] |
| 203124_s_at | 12 | q13.12 | solute carrier family 11 (proton-coupled divalent metal ion |
| | | | transporters), member 2 [Source: HGNC Symbol; Acc: 10908] |
| 1570336_at | 3 | q29 | 3-hydroxybutyrate dehydrogenase, type 1 [Source: HGNC |
| | | | Symbol; Acc: 1027] |
| 1553034_at | 1 | q43 | serologically defined colon cancer antigen 8 [Source: HGNC |
| | | | Symbol; Acc: 10671] |
| 219076_s_at | 12 | q24.33 | peroxisomal membrane protein 2, 22 kDa [Source: HGNC |
| | | | Symbol; Acc: 9716] |
| 230467_at | 1 | p36.33 | transmembrane protein 52 [Source: HGNC Symbol; Acc: 27916] |
| 226111_s_at | 12 | q13.13 | zinc finger protein 385A [Source: HGNC Symbol; Acc: 17521] |
| 231579_s_at | 17 | q25.3 | TIMP metallopeptidase inhibitor 2 [Source: HGNC |
| | | | Symbol; Acc: 11821] |
| 202007_at | 1 | q42.3 | nidogen 1 [Source: HGNC Symbol; Acc: 7821] |
| 207689_at | 11 | q13.2 | T-box 10 [Source: HGNC Symbol; Acc: 11593] |
| 201681_s_at | 10 | q22.3 | discs, large homolog 5 (Drosophila) [Source: HGNC |
| | | | Symbol; Acc: 2904] |
| 212761_at | 10 | q25.2 | transcription factor 7-like 2 (T-cell specific, HMG-box) |
| | | | [Source: HGNC Symbol; Acc: 11641] |
| 224983_at | 4 | q21.1 | scavenger receptor class B, member 2 [Source: HGNC |
| | | | Symbol; Acc: 1665] |
| 218706_s_at | 5 | q23.2 | GRAM domain containing 3 [Source: HGNC Symbol; Acc: 24911] |
| 210165_at | 16 | p13.3 | deoxyribonucleoase I [Source: HGNC Symbol; Acc: 2956] |
| 203595_s_at | 10 | q23.31 | interferon-induced protein with tetratricopeptide repeats 5 |
| | | | [Source: HGNC Symbol; Acc: 13328] |
| 1558431_at | 16 | p13.3 | NHL repeat containing 4 [Source: HGNC Symbol; Acc: 26700] |
| 225133_at | 4 | p14 | Kruppel-like factor 3 (basic) [Source: HGNC Symbol; Acc: 16516] |
| 210122_at | 16 | p13.13 | protamine 2 [Source: HGNC Symbol; Acc: 9448] |
| 204396_s_at | 10 | q26.11 | G protein-coupled receptor kinase 5 [Source: HGNC |
| | | | Symbol; Acc: 4544] |
| 1569270_at | NA | NA | NA |
| 210105_s_at | 6 | q21 | FYN oncogene related to SRC, FGR, YES [Source: HGNC |
| | | | Symbol; Acc: 4037] |
| 201218_at | 10 | q26.13 | C-terminal bidning protein 2 [Source: HGNC Symbol; Acc: 2495] |
| 203973_s_at | 8 | q11.21 | CCAAT/enhancer binding protein (C/EBP), delta [Source: HGNC |
| | | | Symbol; Acc: 1835] |
| 243038_at | 2 | q23.3 | PNA binding motif protein 43 [Source: HGNC Symbol; Acc: 24790] |
| 212667_at | 5 | q33.1 | secreted protein, acidic, cysteine-rich (osteonectin) [Source: HGNC |
| | | | Symbol; Acc: 11219] |
| 209341_s_at | 8 | p11.21 | inhibitor of kappa light polypeptide gene enhancer in B-cells, |
| | | | kinase beta [Source: HGNC Symbol; Acc: 5960] |
| 232781_at | 1 | q25.2 | LIM homeobox 4 [Source: HGNC Symbol; Acc: 21734] |
| 202336_s_at | 5 | q21.1 | peptidylglycine alpha-amidating monooxygenase [Source: HGNC |
| | | | Symbol; Acc: 8596] |
| 216151_x_at | 1 | p36.12 | chymotrypsin-like elastase family, member 38 [Source: HGNC |
| | | | Symbol; Acc: 15945] |
| 1553780_at | 14 | q32.33 | long intergenic non-protein coding RNA 638 [Source: HGNC |
| | | | Symbol; Acc: 28325] |
| 212298_at | 10 | p11.22 | neuropilin 1 [Source: HGNC Symbol; Acc: 8004] |
| 1564386_at | 9 | q31.3 | thioredoxin domain containing 8 (spermatozoa) [Source: HGNC |
| | | | Symbol; Acc: 31454] |
| 221773_at | 12 | q23.1 | ELK3, ETS-domain protein (SRF accessory protein 2) [Source: HGNC |
| | | | Symbol; Acc: 3325] |
| 207116_s_at | 19 | q13.12 | glyceraldehyde-3-phosphate dehydrogenase, spermatogenic |
| | | | [Source: HGNC Symbol; Acc: 24864] |
| 208816_x_at | 9 | p13.3 | annexin A2 pseudogene 2 [Source: HGNC Symbol; Acc: 539] |
| 225188_at | 2 | q33.2 | Ras association (RalGDS/AF-6) and pleckstrin homology domains 1 |
| | | | [Source: HGNC Symbol; Acc: 14436] |
| 1552564_at | NA | NA | NA |
| 212779_at | 4 | q27 | KIAA1109 [Source: HGNC Symbol; Acc: 26953] |
| 221748_s_at | 2 | q35 | tensin 1 [Source: HGNC Symbol; Acc: 11973] |
| 1552811_at | 16 | p13.3 | WAP, follistatin/kazal, immunoglobulin, kunitz and netrin domain |
| | | | containing 1 [Source: HGNC Symbol; Acc: 30912] |
| 244543_s_at | 12 | q13.12 | BCDIN3D antisense RNA 1 [Source: HGNC Symbol; Acc: 44113] |
| 1555124_at | NA | NA | NA |
| 213379_at | 4 | q21.23 | coenzyme Q2 4-hydroxybenzoate polyprenyltransferase |
| | | | [Source: HGNC Symbol; Acc: 25223] |
| 211684_s_at | 2 | q31.1 | dynein, cystoplasmic 1, intermediate chain 2 [Source: HGNC |
| | | | Symbol; Acc: 2964] |
| 201739_at | 6 | q23.2 | serum/glucocorticoid regulated kinase 1 [Source: HGNC |
| | | | Symbol; Acc: 10810] |
| 209090_s_at | 1 | p22.3 | SH3-domain GRB2-like endophilin B1 [Source: HGNC |
| | | | Symbol; Acc: 10833] |
| 225171_at | 6 | q22.33 | Rho GTPase activating protein 18 [Source: HGNC |
| | | | Symbol; Acc: 21035] |
| 204517_at | 5 | q23.2 | peptidylprolyl isomerase C (cyclophilin C) [Source: HGNC |
| | | | Symbol; Acc: 9256] |
| 213923_at | 3 | q25.2 | RAP2B, member of RAS oncogene family [Source: HGNC |
| | | | Symbol; Acc: 9862] |
| 201590_x_at | 15 | q22.2 | annexin A2 [Source: HGNC Symbol; Acc: 537] |
| 202202_s_at | 6 | q21 | laminin, alpha 4 [Source: HGNC Symbol; Acc: 6484] |
| 218718_at | 4 | q32.1 | platelet derived growth factor C [Source: HGNC Symbol; Acc: 8801] |
| 1553541_at | 1 | q23.3 | LIM homeobox transcription factor 1, alpha [Source: HGNC |
| | | | Symbol; Acc: 6653] |
| 2061414_s_at | 2 | p25.1 | ArfGAP with SH3 domain, ankyrin repeat and PH domain 2 |
| | | | [Source: HGNC Symbol; Acc: 2721] |
| 1553178_a_at | 22 | q13.1 | somatostatin receptor 3 [Source: HGNC Symbol; Acc: 11332] |
| 212169_at | 7 | p14.3 | FK506 binding protein 9, 63 kDa [Source: HGNC Symbol; Acc: 3725] |
| 209348_s_at | 16 | q23.2 | v-maf avian musculoaponeurotic fibrosarcoma oncogene homolog |
| | | | [Source: HGNC Symbol; Acc: 6776] |
| 202011_at | 15 | q13.1 | tight junction protein 1 [Source: HGNC Symbol; Acc: 11827] |
| 226823_at | 1 | p35.3 | phosphatase and actin regulator 4 [Source: HGNC |
| | | | Symbol; Acc: 25793] |
| 201375_s_at | 8 | p12 | protein phosphatase 2, catalytic subunit, beta isozyme |
| | | | [Source: HGNC Symbol; Acc: 9300] |
| 1558289_at | 3 | p21.1 | RFT1 homolog (S. cerevisiae) [Source: HGNC Symbol; Acc: 30220] |
| 202808_at | 10 | q24.32 | WW domain binding protein 1-like [Source: HGNC |
| | | | Symbol; Acc: 23510] |
| 227911_at | 18 | p11.31 | Rho GTPase activating protein 28 [Source: HGNC |
| | | | Symbol; Acc: 25509] |
| 208034_s_at | 13 | q34 | protein Z, vitamin K-dependent plasma glycoprotein [Source: HGNC |
| | | | Symbol; Acc: 9460] |
| 1553444_a_at | 1 | p36.22 | chromosome 1 open reading frame 127 [Source: HGNC |
| | | | Symbol; Acc: 26730] |
| 220077_at | 22 | q13.2 | coiled-coil domain containing 134 [Source: HGNC |
| | | | Symbol; Acc: 26185] |
| 215756_at | NA | NA | NA |
| 206049_at | 1 | q24.2 | selectin P (granule membrane protein 140 kDa, antigen CD62) |
| | | | [Source: HGNC Symbol; Acc: 10721] |
| 202357_s_at | 6 | p21.33 | complement factor B [Source: HGNC Symbol; Acc: 1037] |
| 217497_at | 22 | q13.33 | thymidine phosphorylase [Source: HGNC Symbol; Acc: 3148] |
| 204034_at | 19 | q13.31 | ethylmalonic encephalopathy 1 [Source: HGNC Symbol; Acc: 23287] |
| 210139_s_at | 17 | p12 | peripheral myelin protein 22 [Source: HGNC Symbol; Acc: 9118] |
| 242226_at | NA | NA | NA |
| 204114_at | 14 | q22.1 | nidogen 2 (osteonidogen) [Source: HGNC Symbol; Acc: 13389] |
| 207159_x_at | 19 | p13.11 | CREB regulated transcription coactivator 1 [Source: HGNC |
| | | | Symbol; Acc: 16062] |
| 1564072_at | 7 | q22.1 | myosin, heavy chain 16 pseudogene [Source: HGNC |
| | | | Symbol; Acc: 31038] |
| 200878_at | 2 | p21 | endothelial PAS domain protein 1 [Source: HGNC Symbol; Acc: 3374] |
| 206112_at | 7 | q31.31 | ankyrin repeat domain 7 [Source: HGNC Symbol; Acc: 18588] |
| 202446_s_at | 3 | q24 | phospholipid scramblase 1 [Source: HGNC Symbol; Acc: 9092] |
| 221139_s_at | 12 | q13.13 | cysteine sulfinic acid decarboxylase [Source: HGNC |
| | | | Symbol; Acc: 18966] |
| 244488_at | 20 | q13.33 | LSM14B, SCD6 homolog B (S. cerevisiae) [Source: HGNC |
| | | | Symbol; Acc: 15887] |
| 221012_s_at | 10 | q13.33 | tripartite motif containing 8 [Source: HGNC Symbol; Acc: 15579] |
| 1555832_s_at | 10 | p15.1 | Kruppel-like factor 6 [Source: HGNC Symbol; Acc: 2235] |
| 212859_x_at | 16 | q12.2 | metallothionein 1E [Source: HGNC Symbol; Acc: 7397] |
| 203729_at | 19 | q13.33 | epithelial membrane protein 3 [Source: HGNC Symbol; Acc: 3335] |
| 243022_at | NA | NA | NA |
| 223630_at | 7 | q36.3 | chromosome 7 open reading frame 13 [Source: HGNC |
| | | | Symbol; Acc: 17126] |
| 209238_at | 11 | q12.1 | syntaxin 3 [Source: HGNC Symbol; Acc: 11438] |
| 207366_at | 20 | q13.12 | potassium voltage-gated channel, delayed-rectifier, subfamily S, |
| | | | member 1 [Source: HGNC Symbol; Acc: 6300] |
| 1569532_a_at | 9 | q34.3 | lipocalin 15 [Source: HGNC Symbol; Acc: 33777] |
| 228617_at | 17 | p13.1 | XIAP associated factor 1 [Source: HGNC Symbol; Acc: 30932] |
| 204436_at | 15 | q22.31 | pleckstrin homology domain containing. family O member 2 |
| | | | [Source: HGNC Symbol; Acc: 30026] |
| 208291_s_at | 11 | p15.5 | tyrosine hydroxylase [Source: HGNC Symbol; Acc: 11782] |
| 1555756_a_at | 12 | p13.2 | C-type lectin domain family 7, member A [Source: HGNC |
| | | | Symbol; Acc: 14558] |
| 202381_at | 8 | p11.22 | ADAM metallopeptidase domain 9 [Source: HGNC Symbol; Acc: 216] |
| 202291_s_at | 12 | p12.3 | matrix Gla protein [Source: HGNC Symbol; Acc: 7060] |
| 239119_at | 13 | q32.1 | DNAJC3 antisense RNA 1 (head to head) [Source: HGNC |
| | | | Symbol; Acc: 39808] |
| 213056_at | 3 | p14.1 | FERM domain containing 4B [Source: HGNC Symbol; Acc: 24886] |
| 218162_at | 1 | p13.2 | olfactomedin-like 3 [Source: HGNC Symbol; Acc: 24956] |
| 212845_at | 14 | q22.2 | sterile alpha motif domain containing 4A [Source: HGNC |
| | | | Symbol; Acc: 23023] |
| 240185_at | NA | NA | NA |
| 233843_at | 6 | p21.33 | zinc finger and BTB domain containing 12 [Source: HGNC |
| | | | Symbol; Acc: 19066] |
| 218541_s_at | 8 | p11.21 | chromosome 8 open reading frame 4 [Source: HGNC |
| | | | Symbol; Acc: 1357] |
| 1555722_at | NA | NA | NA |
| 1559005_s_at | 9 | p22.2 | centlein, centrosomal protein [Source: HGNC Symbol; Acc: 23432] |
| 218909_at | 1 | q32.3 | ribosomal protein S6 kinase, 52 kDa, polypeptide 1 [Source: HGNC |
| | | | Symbol; Acc: 10439] |
| 1553067_a_at | 1 | q21.1 | gonadotropin-releasing hormone (type 2) receptor 2 [Source: HGNC |
| | | | Symbol; Acc: 16341] |
| 226384_at | 8 | p11.23 | phosphatidic acid phosphatase type 2 domain containing 1B |
| | | | [Source: HGNC Symbol; Acc: 25026] |
| 212509_s_at | 17 | q25.1 | matrix-remodelling associated 7 [Source: HGNC Symbol; Acc: 7541] |
| 225975_at | 4 | q28.3 | protocadherin 18 [Source: HGNC Symbol; Acc: 14268] |
| 201341_at | 5 | q13.3 | ectodermal-neural cortex 1 (with BTB domain) [Source: HGNC |
| | | | Symbol; Acc: 3345] |
| 203324_s_at | 7 | q31.2 | caveolin 2 [Source: HGNC Symbol; Acc: 1528] |
| 238898_at | NA | NA | NA |
| 212230_at | 1 | p32.2 | phosphatidic acid phosphatase type 2B [Source: HGNC |
| | | | Symbol; Acc: 9229] |
| 218632_at | 1 | p34.1 | HECT domain containing E3 ubiquitin protein ligase 3 |
| | | | [Source: HGNC Symbol; Acc: 26117] |
| 212204_at | 15 | q15.1 | trasnmembrane protein 87A [Source: HGNC Symbol; Acc: 24522] |
| 226022_at | 6 | q24.3 | SAM and SH3 domain containing 1 [Source: HGNC |
| | | | Symbol; Acc: 19182] |
| 224166_at | 5 | q31.3 | solute carrier family 25 (mitochondrial carrier; ornithine |
| | | | transporter) member 3 [Source: HGNC Symbol; Acc: 22921] |
| 242800_at | X | p22.13 | Nance-Horan syndrome (congenital cataracts and dental |
| | | | anomalies) [Source: HGNC Symbol; Acc: 7820] |
| 203477_at | 9 | q22.33 | collagen, type XV, alpha 1 [Source: HGNC Symbol; Acc: 2192] |
| 200857_s_at | 17 | p11.2 | nuclear receptor corepressor 1 [Source: HGNC Symbol; Acc: 7672] |
| 238239_at | 6 | q27 | WD repeat domain 27 [Source: HGNC Symbol; Acc: 21248] |
| 1562089_at | 11 | q12.1 | glycine-N-acyltransferace-like 1 [Source: HGNC Symbol; Acc: 30519] |
| 224996_at | 8 | q12.3 | aspartate beta-hydroxylase [Source: HGNC Symbol; Acc: 757] |
| 223562_at | 22 | q13.31 | parvin, gamma [Source: HGNC Symbol; Acc: 14654] |
| 213353_at | 17 | q24.3 | ATP-binding cassette, sub-family A (ABC1), member 5 |
| | | | [Source: HGNC Symbol; Acc: 35] |
| 209593_s_at | 9 | q34.11 | torsin family 1, member B (torsin B) [Source: HGNC |
| | | | Symbol; Acc: 11995] |
| 207649_at | 17 | q21.2 | keratin 37 [Source: HGNC Symbol; Acc: 6455] |
| 32626_at | 17 | q25.3 | N-sulfoglucosamine sulfohydrolase [Source: HGNC |
| | | | Symbol; Acc: 10818] |
| 1559272_at | 16 | q22.1 | exocyst complex component 3-like 1 [Source: HGNC |
| | | | Symbol; Acc: 27540] |
| 234994_at | 6 | q23.1 | transmembrane protein 200A [Source: HGNC Symbol; Acc: 21075] |
| 1556822_s_at | 19 | q13.43 | zinc finger protein 837 [Source: HGNC Symbol; Acc: 25164] |
| 211456_x_at | NA | NA | NA |
| 215328_at | 2 | p23.3 | EFR3 homolog B (S. cerevisiae) [Source: HGNC Symbol; Acc: 29155] |
| 203501_at | 8 | q22.1 | carboxypeptidase Q [Source: HGNC Symbol; Acc: 16910] |
| 226155_at | 10 | q25.3 | family with sequence similarity 160, member B1 [Source: HGNC |
| | | | Symbol; Acc: 29320] |
| 1552932_at | 11 | p15.5 | NLR family, pyrin domain containing 6 [Source: HGNC |
| | | | Symbol; Acc: 22944] |
| 1552258_at | 2 | p11.2 | long intergenic non-protein coding RNA 152 [Source: HGNC |
| | | | Symbol; Acc: 28717] |
| 238025_at | 16 | q23.1 | mixed lineage kinase domain-like [Source: HGNC |
| | | | Symbol; Acc: 26617] |
| 219460_s_at | 2 | q11.2 | transmembrane protein 127 [Source: HGNC Symbol; Acc: 26038] |
| 225522_at | 2 | p13.3 | AP2 associated kinase 1 [Source: HGNC Symbol; Acc: 19679] |
| 217757_at | 12 | p13.31 | alpha-2-macroglobulin [Source: HGNC Symbol; Acc: 7] |
| 223168_at | 1 | q42.13 | ras homolog family member U [Source: HGNC Symbol; Acc: 17794] |
| 1561615_s_at | 2 | p22.1 | solute carrier family 8 (sodium/calcium exchanger), member 1 |
| | | | [Source: HGNC Symbol; Acc: 11068] |
| 203758_at | 4 | q32.1 | cathepsin O [Source: HGNC Symbol; Acc: 2542] |
| 226552_at | 9 | q34.11 | immediately response 5-like [Source: HGNC Symbol; Acc: 23679] |
| 202766_s_at | 15 | q21.1 | fibrillin 1 [Source: HGNC Symbol; Acc: 3603] |
| 225629_s_at | 17 | p13.1 | zinc finger and BTB domain containing 4 [Source: HGNC |
| | | | Symbol; Acc: 23847] |
| 212136_at | 1 | q32.1 | ATPase, Ca [symbol] transporting, plasma membrane 4 [Source: HGNC |
| | | | Symbol; Acc: 817] |
| 1555881_s_at | 10 | q24.31 | leucine zipper, putative tumor suppressor 2 [Source: HGNC |
| | | | Symbol; Acc: 29381] |
| 57715_at | 10 | q24.33 | calcium homeostasis modulator 2 [Source: HGNC |
| | | | Symbol; Acc: 23493] |
| 226601_at | 1 | p21.2 | solute carrier family 30 (zinc transporter), membrane 7 [Source: HGNC |
| | | | Symbol; Acc: 19306] |
| 217890_s_at | 11 | p15.3 | parvin, alpha [Source: HGNC Symbol; Acc: 14652] |
| 207624_s_at | X | p11.4 | retinitis pigmentosa GTPase regulator [Source: HGNC |
| | | | Symbol; Acc: 10295] |
| 212372_at | 17 | p13.1 | myosin, heavy chain 10, non-muscle [Source: HGNC |
| | | | Symbol; Acc: 7568] |
| 226820_at | 1 | p35.1 | zinc finger protein 362 [Source: HGNC Symbol; Acc: 18079] |
| 202551_s_at | 2 | p22.3 | cysteine rich transmembrane BMP regulator 1 (chordin-like) |
| | | | [Source: HGNC Symbol; Acc: 2359] |
| 208030_s_at | 4 | p16.3 | adducin 1 (alpha) [Source: HGNC Symbol; Acc: 243] |
| 206618_at | 2 | q12.1 | interleukin 18 receptor 1 [Source: HGNC Symbol; Acc: 5988] |
| 201212_at | 14 | q32.12 | legumain [Source: HGNC Symbol; Acc: 9472] |
| 208782_at | 3 | q13.33 | follistatin-like 1 [Source: HGNC Symbol; Acc: 3972] |
| 209191_at | 18 | p11.21 | tubulin, beta 6 class V [Source: HGNC Symbol; Acc: 20776] |
| 206875_s_at | 10 | q24.33 | STE20-like kinase [Source: HGNC Symbol; Acc: 11088] |
| 212990_at | 21 | q22.11 | synaptojanin 1 [Source: HGNC Symbol; Acc: 11503] |
| 204193_at | 22 | q13.33 | choline kinase beta [Source: HGNC Symbol; Acc: 1938] |
| 203243_s_at | 4 | q22.3 | PD2 and LIM domain 5 [Source: HGNC Symbol; Acc: 17468] |
| 227554_at | 7 | q21.11 | MAGI2 antisense RNA 3 [Source: HGNC Symbol; Acc: 40862] |
| 224616_at | 16 | q22.1 | dynein, cytoplasmic 1, light intermediate chain 2 [Source: HGNC |
| | | | Symbol; Acc: 2966] |
| 218901_at | 3 | q24 | phospholipid scramble 4 [Source: HGNC Symbol; Acc: 16497] |
| 209050_s_at | 9 | q34.2 | ral guanine nucleotide dissociation stimulator [Source: HGNC |
| | | | Symbol; Acc: 9842] |
| 209472_at | 1 | p22.2 | cysteine conjugate-beta lyase 2 [Source: HGNC Symbol; Acc: 33238] |
| 227542_at | 18 | q22.2 | suppressor of cytokine signaling 6 [Source: HGNC |
| | | | Symbol; Acc: 16833] |
| 204039_at | 19 | q13.11 | CCAAT/enhancer binding protein (C/EBP), alpha [Source: HGNC |
| | | | Symbol; Acc: 1833] |
| 212586_at | 5 | q15 | calpastatin [Source: HGNC Symbol; Acc: 1515] |
| 201280_s_at | 5 | p13.1 | Dab, mitogen-responsive phosphoprotein, homolog 2 (Drosophila) |
| | | | [Source: HGNC Symbol; Acc: 2662] |
| 201599_at | 10 | q26.13 | ornithine aminotransferase [Source: HGNC Symbol; Acc: 8091] |
| 225334_at | 10 | q24.32 | chromosome 10 open reading frame 32 [Source: HGNC |
| | | | Symbol; Acc: 23516] |
| 201334_s_at | 11 | q23.3 | Rho guanine nucleotide exchange factor (GEF) 12 [Source: HGNC |
| | | | Symbol; Acc: 14193] |
| 229041_s_at | 21 | q22.3 | ITGB2 antisense RNA 1 [Source: HGNC Symbol; Acc: 44304] |
| 212124_at | 10 | q22.3 | zinc finger, MIZ-type containing 1 [Source: HGNC |
| | | | Symbol; Acc: 16493] |
| 204863_s_at | 5 | q11.2 | interleukin 6 signal transducer (gp130, oncostatin M receptor) |
| | | | [Source: HGNC Symbol; Acc: 6021] |
| 215235_at | 9 | q34.11 | spectrim, alpha, non-erythrocytic 1 [Source: HGNC |
| | | | Symbol; Acc: 11273] |
| 212606_at | 4 | q21.23 | WD repeat and FYVE domain containing 3 [Source: HGNC |
| | | | Symbol; Acc: 20751] |
| 212601_at | 17 | p13.2 | zinc finger, ZZ-type with EF-hand domain 1 [Source: HGNC |
| | | | Symbol; Acc: 29027] |
| 228577_x_at | 1 | p22.3 | outer dense fiber of sperm tails 2-like [Source: HGNC |
| | | | Symbol; Acc: 29225] |
| 227776_at | 11 | q13.5 | alkaline ceramidase 3 [Source: HGNC Symbol; Acc: 16066] |
| 201146_at | 2 | q31.2 | nuclear factor (erythroid-derived 2)-like 2 [Source: HGNC |
| | | | Symbol; Acc: 7782] |
| 214807_at | NA | NA | NA |
| 201185_at | 10 | q26.13 | HtrA serine peptidase 1 [Source: HGNC Symbol; Acc: 9476] |
| 209120_at | 15 | q26.2 | nuclear receptor subfamily 2, group F, member 2 [Source: HGNC |
| | | | Symbol; Acc: 7976] |
| 226021_at | 8 | q21.11 | retinol dehydrogenase 10 (all-trans) [Source: HGNC |
| | | | Symbol; Acc: 19975] |
| 224480_s_at | 4 | q21.23 | 1-acylglycerol-3-phosphate O-acyltransferase 9 [Source: HGNC |
| | | | Symbol; Acc: 28157] |
| 212077_at | 7 | q33 | caldesmon 1 [Source: HGNC Symbol; Acc: 1441] |
| 204745_x_at | 16 | q13 | metallothionein 1G [Source: HGNC Symbol; Acc: 7399] |
| 227930_at | 1 | p34.3 | argonaute RISC catalytic component 4 [Source: HGNC |
| | | | Symbol; Acc: 18424] |
| 212636_at | 6 | q26 | QKI, KH domain containing, RNA binding [Source: HGNC |
| | | | Symbol; Acc: 21100] |
| 202609_at | 12 | p12.3 | epidermal growth factor receptor pathway substrate 8 |
| | | | [Source: HGNC Symbol; Acc: 3420] |
| 207791_s_at | 2 | p14 | RAB1A, member RAS oncogene family [Source: HGNC |
| | | | Symbol; Acc: 9758] |
| 226377_at | 19 | p13.3 | nuclear factor I/C (CCAAT-binding transcription factor) |
| | | | [Source: HGNC Symbol; Acc: 7786] |
| 212607_at | 1 | q44 | v-akt murine thymoma viral oncogene homolog 3 [Source: HGNC |
| | | | Symbol; Acc: 393] |
| 220122_at | 5 | q15 | multiple C2 domains, transmembrane 1 [Source: HGNC |
| | | | Symbol; Acc: 26183] |
| 225941_at | 3 | p13 | eukaryotic translation initiation factor 4E family member 3 |
| | | | [Source: HGNC Symbol; Acc: 31837] |
| 65635_at | 17 | q25.3 | endo-beta-N-acetylglucosaminidase [Source: HGNC |
| | | | Symbol; Acc: 24622] |
| 202897_at | 20 | p13 | signal-regulatory protein alpha [Source: HGNC Symbol; Acc: 9662] |
| 213817_at | 12 | q14.3 | interleukin-1 receptor-associated kinase 3 [Source: HGNC |
| | | | Symbol; Acc: 17020] |
| 204703_at | 13 | q12.11 | intraflagellar transport 88 homolog (Chlamydomonas) |
| | | | [Source: HGNC Symbol; Acc: 20606] |
| 201029_s_at | X | p22.33 | CD99 molecule [Source: HGNC Symbol; Acc: 7032] |
| 201105_at | 22 | q13.1 | lectin, galactoside-binding, soluble, 1 [Source: HGNC |
| | | | Symbol; Acc: 6561] |
| 228666_at | 15 | q26.1 | chromosome 15 open reading frame 38 [Source: HGNC |
| | | | Symbol; Acc: 28782] |
| 213733_at | 19 | p13.2 | myosin IF [Source: HGNC Symbol; Acc: 7600] |
| 225162_at | 4 | q31.3 | SH3 domain containing 19 [Source: HGNC Symbol; Acc: 30418] |
| 221766_s_at | 6 | q14.1 | family with sequence similarity 46, member A [Source: HGNC |
| | | | Symbol; Acc: 18345] |
| 235256_s_at | 2 | p22.1 | galactose mutarotase (aldose 1-epimerase) [Source: HGNC |
| | | | Symbol; Acc: 24063] |
| 201417_at | 6 | p22.3 | SRY (sex determining region Y)-box 4 [Source: HGNC |
| | | | Symbol; Acc: 11200] |
| 201963_at | 4 | q35.1 | acyl-CoA synthetase long-chain family member 1 [Source: HGNC |
| | | | Symbol; Acc: 3569] |
| 218983_at | 12 | p13.31 | complement component 1, r subcomponent-like [Source: HGNC |
| | | | Symbol; Acc: 21265] |
| 225442_at | 1 | q23.3 | discoidin domain receptor tyrosine kinase 2 [Source: HGNC |
| | | | Symbol; Acc: 2731] |
| 225128_at | 11 | q22.3 | KDEL (Lys-Asp-Glu-Leu) containing 2 [Source: HGNC |
| | | | Symbol; Acc: 28496] |
| 225913_at | NA | NA | NA |
| 238477_at | 17 | p13.2 | kinesin family member 1C [Source: HGNC Symbol; Acc: 6317] |
| 1557236_at | 22 | q12.3 | apolipoprotein L, 6 [Source: HGNC Symbol; Acc: 14870] |
| 224764_at | 10 | p12.1 | Rho GTPase activating protein 21 [Source: HGNC |
| | | | Symbol; Acc: 23725] |
| 204568_at | 14 | q22.3 | autophagy related 14 [Source: HGNC Symbol; Acc: 19962] |
| 202598_at | 1 | q21.3 | S100 calcium binding protein A13 [Source: HGNC |
| | | | Symbol; Acc: 10490] |
| 218815_s_at | 1 | p36.21 | transmembrane protein 51 [Source: HGNC Symbol; Acc: 25483] |
| 204214_s_at | 6 | q24.3 | RAB32, member RAS oncogene family [Source: HGNC |
| | | | Symbol; Acc: 9772] |
| 226152_at | 14 | q32.11 | tetratricopeptide repeat domain 7B [Source: HGNC |
| | | | Symbol; Acc: 19858] |
| 205590_at | 15 | q14 | RAS guanyl releasing protein 1 (calcium and DAG-regulated) |
| | | | [Source: HGNC Symbol; Acc: 9878] |
| 1555579_s_at | 18 | p11.23 | protein tyrosine phosphatase, receptor type, M [Source: HGNC |
| | | | Symbol; Acc: 9675] |
| 218486_at | 2 | p25.1 | Kruppel-factor 11 [Source: HGNC Symbol; Acc: 11811] |
| 204451_at | 7 | q21.13 | frizzled family receptor 1 [Source: HGNC Symbol; Acc: 4038] |
| 226186_at | 15 | q21.2 | tropomodulin 2 (neuronal) [Source: HGNC Symbol; Acc: 11872] |
| 225288_at | 9 | q32 | collagen, type XXVII, alpha 1 [Source: HGNC Symbol; Acc: 22986] |
| 225163_at | 10 | p13 | FERM domain containing 4A [Source: HGNC Symbol; Acc: 25491] |
| 221541_at | 16 | q24.3 | cystein-rich secretary protein LCCL domain containing 2 |
| | | | [Source: HGNC Symbol; Acc: 25248] |
| 230480_at | 11 | q21 | piwi-like RNA-mediated gene silencing 4 [Source: HGNC |
| | | | Symbol; Acc: 18444] |
| 227444_at | X | q22.1 | armadillo repeat containing, X-linked 4 [Source: HGNC |
| | | | Symbol; Acc: 28615] |
| 218285_s_at | 4 | q24 | 3-hydroxybutyrate dehydrogenase, type 2 [Source: HGNC |
| | | | Symbol; Acc: 32389] |
| 209687_at | 10 | q11.21 | chemokine (C-X-C motif) ligand 12 [Source: HGNC |
| | | | Symbol; Acc: 10672] |
| 203261_at | 8 | p12 | dynactin 6 [Source: HGNC Symbol; Acc: 16964] |
| 227001_at | 8 | q22.2 | NIPA-like domain containing 2 [Source: HGNC Symbol; Acc: 25854] |
| 213119_at | 5 | q33.1 | solute carrier family 36 (proton/amino acid symporter), member 1 |
| | | | [Source: HGNC Symbol; Acc: 18761] |
| 201089_at | 8 | p21.3 | ATPase, H transporting, lysosomal 56/58 kDa, V1 subunit B2 |
| | | | [Source: HGNC Symbol; Acc: 854] |
| 228937_at | 13 | q14.11 | laccase (multicopper oxidoreductase) domain containing 1 |
| | | | [Source: HGNC Symbol; Acc: 26789] |
| 219666_at | 11 | q12.2 | membrane-spanning 4-domains, subfamily A, member 6A |
| | | | [Source: HGNC Symbol; Acc: 13375] |
| 209160_at | 10 | p15.1 | aldo-keto reductase family 1, member C3 [Source: HGNC |
| | | | Symbol; Acc: 386] |
| 203688_at | 4 | q22.1 | polycystic kidney disease 2 (autosomal dominant) [Source: HGNC |
| | | | Symbol; Acc: 9009] |
| 209379_s_at | 10 | q23.1 | coiled-coil serine-rich protein 2 [Source: HGNC Symbol; Acc: 29197] |
| 202973_x_at | 4 | q22.1 | family with sequence similarity 13, member A [Source: HGNC |
| | | | Symbol; Acc: 19367] |
| 222999_s_at | 1 | p36.33 | cyclin L2 [Source: HGNC Symbol; Acc: 20570] |
| 32811_at | 17 | p13.3 | myosin IC [Source: HGNC Symbol; Acc: 7597 |
| 213737_x_at | 15 | q11.2 | golgin A8 family, member I [Source: HGNC Symbol; Acc: 26660] |
| 202351_at | 2 | q32.1 | integrin, alpha V [Source: HGNC Symbol; Acc: 6150] |
| 226066_at | 3 | p14.1 | microphthalmia-associated transcription factor [Source: HGNC |
| | | | Symbol; Acc: 7105] |
| 212453_at | 10 | q22.1 | KIAA1279 [Source: HGNC Symbol; Acc: 23419] |
| 223276_at | 5 | q33.1 | small integral membrane protein 3 [Source: HGNC |
| | | | Symbol; Acc: 30248] |
| 63825_at | 15 | q26.1 | abhydrolase domain containing 2 [Source: HGNC |
| | | | Symbol; Acc: 18717] |
| 203817_at | 4 | q32.1 | guanylate cyclase 1, soluble, beta 3 [Source: HGNC |
| | | | Symbol; Acc: 4687] |
| 232090_at | 1 | q24.3 | DNM3 opposite strand/antisense RNA [Source: HGNC |
| | | | Symbol; Acc: 41228] |
| 218292_s_at | 7 | q36.1 | protein kinase, AMP-activated, gamma 2 non-catalytic subunit |
| | | | [Source: HGNC Symbol; Acc: 9386] |
| 1558173_a_at | 1 | p36.12 | leucine zipper protein 1 [Source: HGNC Symbol; Acc: 14985] |
| 201438_at | 2 | q37.3 | collagen, type VI, alpha 3 [Source: HGNC Symbol; Acc: 2213] |
| 235458_at | 5 | q33.3 | hepatitis A virus cellular receptor 2 [Source: HGNC |
| | | | Symbol; Acc: 18437] |
| 223393_s_at | 19 | q12 | teashirt zinc finger homeobox 3 [Source: HGNC Symbol; Acc: 30700] |
| 215706_x_at | 7 | q34 | zyxin [Source: HGNC Symbol; Acc: 13200] |
| 219315_s_at | 16 | p13.3 | transmembrane protein 204 [Source: HGNC Symbol; Acc: 14158] |
| 201296_s_at | 17 | q11.1 | WD repeat and SOCS box containing 1 [Source: HGNC |
| | | | Symbol; Acc: 19221] |
| 232231_at | 6 | p21.1 | runt-related transcription factor 2 [Source: HGNC |
| | | | Symbol; Acc: 10472] |
| 226225_at | 5 | q22.2 | mutated in colorectal cancers [Source: HGNC Symbol; Acc: 6935] |
| 214660_at | 5 | q11.2 | integrin, alpha 1 [Source: HGNC Symbol; Acc: 6134] |
| 216903_s_at | 10 | q22.1 | mitochondrial calcium uptake 1 [Source: HGNC Symbol; Acc: 1530] |
| 215111_s_at | 13 | q14.11 | TSC22 domain family, member 1 [Source: HGNC Symbol; Acc: 16826] |
| 200782_at | 4 | q27 | annexin A5 [Source: HGNC Symbol; Acc: 543] |
| 226771_at | 1 | q21.3 | ATPase, aminophospholipid transporter, class I, type 8B, member 2 |
| | | | [Source: HGNC Symbol; Acc: 13534] |
| 212185_x_at | 16 | q12.2 | metallothionein 2A [Source: HGNC Symbol; Acc: 7406] |
| 200986_at | 11 | q12.1 | serpin peptidase inhibitor, clade G (C1 inhibitor), member 1 |
| | | | [Source: HGNC Symbol; Acc: 1228] |
| 221269_s_at | 1 | p36.11 | SH3 domain binding glutamic acid-rich protein like 3 [Source: HGNC |
| | | | Symbol; Acc: 15568] |
| 202081_at | 19 | p13.2 | immediate early response 2 [Source: HGNC Symbol; Acc: 28871] |
| 206995_x_at | 17 | p13.3 | scavenger receptor class F, member 1 [Source: HGNC |
| | | | Symbol; Acc: 16820] |
| 204963_at | 12 | p12.1 | sarcospan [Source: HGNC Symbol; Acc: 11322] |
| 202192_s_at | 17 | p13.1 | growth arrest-specific 7 [Source: HGNC Symbol; Acc: 4169] |
| 224747_at | 15 | q24.2 | ubiquitin-conjugating enzyme E2Q family member 2 [Source: HGNC |
| | | | Symbol; Acc: 19248] |
| 202136_at | 10 | p15.3 | zinc finger, MYND-type containing 11 [Source: HGNC |
| | | | Symbol; Acc: 16966] |
| 209970_x_at | 11 | q22.3 | caspase 1, apoptosis-related cysteine peptidase [Source: HGNC |
| | | | Symbol; Acc: 1499] |
| 234987_at | 20 | q11.23 | SAM domain and HD domain 1 [Source: HGNC Symbol; Acc: 15925] |
| 205173_x_at | 1 | p13.1 | CD58 molecule [Source: HGNC Symbol; Acc: 1688] |
| 229732_at | 19 | p13.2 | zinc finger protein 823 [Source: HGNC Symbol; Acc: 30936] |
| 37408_at | 17 | q23.2 | mannose receptor, C type 2 [Source: HGNC Symbol; Acc: 16875] |
| 208922_s_at | 11 | q12.3 | nuclear RNA export factor 1 [Source: HGNC Symbol; Acc: 8071] |
| 200766_at | 11 | p15.5 | cathepsin D [Source: HGNC Symbol; Acc: 2529] |
| 209129_at | 7 | q22.1 | thyroid hormone receptor interactor 6 [Source: HGNC |
| | | | Symbol; Acc: 12311] |
| 228728_at | 7 | q31.31 | cadherin-like and PC-esterase domain containing 1 [Source: HGNC |
| | | | Symbol; Acc: 26159] |
| 223028_s_at | 6 | q25.3 | sorting nexin 9 [Source: HGNC Symbol; Acc: 14973] |
| 214464_at | 1 | q42.13 | CDC42 binding protein kinase alpha (DMPK-like) [Source: HGNC |
| | | | Symbol; Acc: 1737] |
| 204059_s_at | 6 | q14.2 | malic enzyme 1, NADP( )-dependent, cytosolic [Source: HGNC |
| | | | Symbol; Acc: 6983] |
| 202481_at | 1 | p36.22 | dehydrogenase/reductase (SDR family) member 3 [Source: HGNC |
| | | | Symbol; Acc: 17693] |
| 201426_s_at | 10 | p13 | vimentin [Source: HGNC Symbol; Acc: 12692] |
| 214177_s_at | 1 | q21.3 | pre-B-cell leukemia homeobox interacting protein 1 [Source: HGNC |
| | | | Symbol; Acc: 21199] |
| 218793_s_at | X | p22.13 | sex comb on midleg-like 1 (Drosophila) [Source: HGNC |
| | | | Symbol; Acc: 10580] |
| 201163_s_at | 4 | q12 | insulin-like growth factor binding protein 7 [Source: HGNC |
| | | | Symbol; Acc: 5476] |
| 225406_at | 18 | p11.22 | twisted gastrulation homolog 1 (Drosophila) [Source: HGNC |
| | | | Symbol; Acc: 12429] |
| 209071_s_at | 1 | q23.3 | regulator of G-protein signaling 5 [Source: HGNC |
| | | | Symbol; Acc: 10001] |
| 204206_at | 17 | p13.3 | MNT, MAX dimerization protein [Source: HGNC Symbol; Acc: 7188] |
| 226026_at | 3 | q21.1 | disrupted in renal carcinoma 2 [Source: HGNC Symbol; Acc: 16628] |
| 214683_s_at | 2 | q33.1 | CDC-like kinase 1 [Source: HGNC Symbol; Acc: 2068] |
| 218095_s_at | 4 | q12 | transmembrane protein 165 [Source: HGNC Symbol; Acc: 30760] |
| 219316_s_at | 14 | q24.3 | feline leukemia virus subgroup C cellular receptor family, member |
| | | | 2 [Source: HGNC Symbol; Acc: 20105] |
| 226763_at | 2 | q31.2 | SEC14 and spectrin domains 1 [Source: HGNC Symbol; Acc: 18379] |
| 227361_at | 17 | p12 | heparan sulfate (glucosamine) 3-O-sulfotransferase 3B1 |
| | | | [Source: HGNC Symbol; Acc: 5198] |
| 205248_at | 21 | q22.12 | dopey family member 2 [Source: HGNC Symbol; Acc: 1291] |
| 211026_s_at | 3 | q21.3 | monoglyceride lipase [Source: HGNC Symbol; Acc: 17038] |
| 212224_at | 9 | q21.13 | aldehyde dehydrogenase 1 family, member A1 [Source: HGNC |
| | | | Symbol; Acc: 402] |
| 201215_at | X | q23 | plastin 3 [Source: HGNC Symbol; Acc: 9091] |
| 218432_at | 11 | p13 | F-box protein 3 [Source: HGNC Symbol; Acc: 13582] |
| 212428_at | 9 | q31.3 | KIAA0368 [Source: HGNC Symbol; Acc: 29020] |
| 203394_s_at | 3 | q29 | hairy and enhancer of split 1, (Drosophila) [Source: HGNC |
| | | | Symbol; Acc: 5192] |
| 235125_x_at | 1 | p31.1 | family with sequence similarity 73, member A [Source: HGNC |
| | | | Symbol; Acc: 24741] |
| 224690_at | 20 | q13.2 | family with sequence similarity 210, member B [Source: HGNC |
| | | | Symbol; Acc: 16102] |
| 219991_at | 4 | p16.1 | solute carrier family 2 (facilitated glucose transporter), member 9 |
| | | | [Source: HGNC Symbol; Acc: 13446] |
| 214560_at | 19 | q13.41 | formyl peptide receptor 3 [Source: HGNC Symbol; Acc: 3828] |
| 210946_at | 5 | q11.2 | phosphatidic acid phosphatase type 2A [Source: HGNC |
| | | | Symbol; Acc: 9228] |
| 210817_s_at | 17 | q21.32 | calcium binding and coiled-coil domain 2 [Source: HGNC |
| | | | Symbol; Acc: 29912] |
| 208636_at | 14 | q24.1 | actinin, alpha 1 [Source: HGNC Symbol; Acc: 163] |
| 1561226_at | 3 | p21.31 | chemokine (C motif) receptor 1 [Source: HGNC Symbol; Acc: 1625] |
| 229256_at | 11 | q13.4 | phosphoglucomutase 2-like 1 [Source: HGNC Symbol; Acc: 20898] |
| 228131_at | 19 | q13.32 | excision repair cross-complementing rodent repair deficiency, |
| | | | complementation group 1 (includes overlapping antisense |
| | | | sequence) [Source: HGNC Symbol; Acc: 3433] |
| 209310_s_at | 11 | q22.3 | caspase 4, apoptosis-related cysteine peptidase [Source: HGNC |
| | | | Symbol; Acc: 1505] |
| 218729_at | 3 | q25.32 | latexin [Source: HGNC Symbol; Acc: 13347] |
| 227274_at | 14 | q24.2 | synaptojanin 2 binding protein [Source: HGNC Symbol; Acc: 18955] |
| 219147_s_at | 9 | q21.13 | nicotinamide riboside kinase 1 [Source: HGNC Symbol; Acc: 26057] |
| 205083_at | 2 | q33.1 | aldehyde oxidase 1 [Source: HGNC Symbol; Acc: 553] |
| 209109_s_at | X | q22.1 | tetraspanin 6 [Source: HGNC Symbol; Acc: 11858] |
| 225303_at | 1 | q23.1 | kin of IRRE like (Drosophila) [Source: HGNC Symbol; Acc: 15734] |
| 225185_at | 3 | q22.3 | muscle RAS oncogene homolog [Source: HGNC Symbol; Acc: 7227] |
| 203716_s_at | 2 | q24.2 | dipeptidyl-peptidase 4 [Source: HGNC Symbol; Acc: 3009] |
| 204342_at | 1 | p13.3 | solute carrier family 25 (mitochondrial carrier; phosphate carrier), |
| | | | member 24 [Source: HGNC Symbol; Acc: 20662] |
| 202948_at | 2 | q11.2 | interleukin 1 receptor, type I [Source: HGNC Symbol; Acc: 5993] |
| 212511_at | 11 | q14.2 | phosphatidylinositol binding clathrin assembly protein |
| | | | [Source: HGNC Symbol; Acc: 15514] |
| 214429_at | 13 | q12.13 | myotubularin related protein 6 [Source: HGNC Symbol; Acc: 7453] |
| 214021_x_at | 3 | q21.2 | integrin, beta 5 [Source: HGNC Symbol; Acc: 6160] |
| 227624_at | 4 | q24 | tet methylcytosine dioxygenase 2 [Source: HGNC Symbol; Acc: 25941] |
| 225093_at | 6 | q24.2 | utrophin [Source: HGNC Symbol; Acc: 12635] |
| 227379_at | 6 | p22.3 | membrane bound O-acyltransferase domain containing 1 |
| | | | [Source: HGNC Symbol; Acc: 21579] |
| 213139_at | 8 | q11.21 | snail family zinc Finger 2 [Source: HGNC Symbol; Acc: 11094] |
| 212099_at | 2 | p24.1 | ras homolog family member B [Source: HGNC Symbol; Acc: 668] |
| 204894_s_at | 17 | q21.31 | amine oxidase, copper containing 3 [Source: HGNC Symbol; Acc: 550] |
| 219432_at | 4 | p16.2 | Ellis van Creveld syndrome [Source: HGNC Symbol; Acc: 3497] |
| 55065_at | 19 | q13.32 | MAP/microtobule affinity-regulating kinase 4 [Source: HGNC |
| | | | Symbol; Acc: 13538] |
| 226353_at | 15 | q21.2 | signal peptide peptidase like 2A [Source: HGNC Symbol; Acc: 30227] |
| 216598_s_at | 17 | q12 | chemokine (C-C motif) ligand 2 [Source: HGNC Symbol; Acc: 10618] |
| 226568_at | 1 | p13.3 | family with sequence similarity 102, member B [Source: HGNC |
| | | | Symbol; Acc: 27637] |
| 218764_at | 14 | q23.1 | protein kinase C, eta [Source: HGNC Symbol; Acc: 9403] |
| 225984_at | 5 | p13.1 | protein kinase, AMP-activated, alpha 1 catalytic subunit |
| | | | [Source: HGNC Symbol; Acc: 9376] |
| 201542_at | 10 | q22.1 | SAR1 homolog A (S. cerevisiae) [Source: HGNC Symbol; Acc: 10534] |
| 203455_s_at | X | p22.11 | spermidine/spermine N1-acetyltransferase 1 [Source: HGNC |
| | | | Symbol; Acc: 10540] |
| 225922_at | 4 | q32.1 | folliculin interacting protein 2 [Source: HGNC Symbol; Acc: 29280] |
| 218665_at | 11 | q14.2 | frizzled family receptor 4 [Source: HGNC Symbol; Acc: 4042] |
| 221666_s_at | 16 | p11.2 | PYD and CARD domain containing [Source: HGNC |
| | | | Symbol; Acc: 16608] |
| 201058_s_at | 20 | q11.23 | myosin, light chain 9, regulatory [Source: HGNC Symbol; Acc: 15754] |
| 203397_s_at | 2 | q24.3 | UDP-N-acetyl-alpha-D-galactosamine:polypeptide N- |
| | | | acetylgalactosaminyltransferase 3 (GalNAc-T3) |
| | | | [Source: HGNC Symbol; Acc: 4125] |
| 222453_at | 2 | q31.1 | cytochrome b reductase 1 [Source: HGNC Symbol; Acc: 20797] |
| 208146_s_at | 7 | p14.3 | carboxypeptidase, vitellogenic-like [Source: HGNC |
| | | | Symbol; Acc: 14399] |
| 227334_at | 10 | q22.2 | ubiquitin specific peptidase 54 [Source: HGNC Symbol; Acc: 23513] |
| 226534_at | 12 | q21.32 | KIT ligand [Source: HGNC Symbol; Acc: 6343] |
| 229120_s_at | 1 | q21.3 | CDC42 small effector 1 [Source: HGNC Symbol; Acc: 17719] |
| 225066_at | 10 | q26.3 | protein phosphatase 2, regulatory subunit B, delta [Source: HGNC |
| | | | Symbol; Acc: 23732] |
| 211977_at | 9 | q34.11 | G protein-coupled receptor 107 [Source: HGNC Symbol; Acc: 17830] |
| 217967_s_at | 1 | q25.3 | family with sequence similarity 129, member A [Source: HGNC |
| | | | Symbol; Acc: 16784] |
| 207705_s_at | 20 | p11.21 | ninein-like [Source: HGNC Symbol; Acc: 29163] |
| 230029_x_at | 2 | q31.1 | ubiquitin protein ligase E3 component n-recognin 3 (putative) |
| | | | [Source: HGNC Symbol; Acc: 30467] |
| 227236_at | 1 | p13.2 | tetraspanin 2 [Source: HGNC Symbol; Acc: 20659] |
| 202510_s_at | 14 | q32.32 | tumor necrosis factor, alpha-induced protein 2 [Source: HGNC |
| | | | Symbol; Acc: 11895] |
| 202450_s_at | 1 | q21.3 | cathepsin K [Source: HGNC Symbol; Acc: 2536] |
| 226395_at | 8 | p11.21 | hook homolog 3 (Drosophila) [Source: HGNC Symbol; Acc: 23576] |
| 219403_s_at | 4 | q21.23 | heparanase [Source: HGNC Symbol; Acc: 5164] |
| 227623_at | 7 | q21.11 | calcium channel, voltage-dependent, alpha 2/delta subunit 1 |
| | | | [Source: HGNC Symbol; Acc: 1399] |
| 202239_at | 13 | q12.12 | poly (ADP-ribose) polymerase family, member 4 [Source: HGNC |
| | | | Symbol; Acc: 271] |
| 204646_at | 1 | p21.3 | dihydropyrimidine dehydrogenase [Source: HGNC |
| | | | Symbol; Acc: 3012] |
| 209335_at | 12 | q21.33 | decorin [Source: HGNC Symbol; Acc: 2705] |
| 202341_s_at | 4 | q31.3 | tripartite motif containing 2 [Source: HGNC Symbol; Acc: 15974] |
| 203386_at | 13 | q22.2 | TBC1 domain family, member 4 [Source: HGNC Symbol; Acc: 19165] |
| 202465_at | 7 | q22.1 | procollagen C-endopeptidase enhancer [Source: HGNC |
| | | | Symbol; Acc: 8738] |
| 218501_at | 3 | p14.3 | Rho guanine nucleotide exchange factor (GEF) 3 [Source: HGNC |
| | | | Symbol; Acc: 683] |
| 203935_at | 2 | q24.1 | activin A receptor, type I [Source: HGNC Symbol; Acc: 171] |
| 227726_at | 16 | q24.3 | ring finger protein 166 [Source: HGNC Symbol; Acc: 28856] |
| 225351_at | 10 | q26.11 | family with sequence similarity 45, member A [Source: HGNC |
| | | | Symbol; Acc: 31793] |
| 209967_s_at | 10 | p11.21 | cAMP responsive element modulator [Source: HGNC |
| | | | Symbol; Acc: 2352] |
| 217767_at | 19 | p13.3 | complement component 3 [Source: HGNC Symbol; Acc: 1318] |
| 202377_at | 1 | p31.3 | leptin receptor overlapping transcript [Source: HGNC |
| | | | Symbol; Acc: 29447] |
| 212067_s_at | 12 | p13.31 | complement component 1, r subcomponent [Source: HGNC |
| | | | Symbol; Acc: 1246] |
| 206461_x_at | 16 | q13 | metallothionein 1H [Source: HGNC Symbol; Acc: 7400] |
| 212651_at | 10 | q21.2 | Rho-related BTB domain containing 1 [Source: HGNC |
| | | | Symbol; Acc: 18738] |
| 213077_at | 5 | q22.2 | YTH domain containing 2 [Source: HGNC Symbol; Acc: 24721] |
| 238617_at | 1 | q44 | kinesin family member 26B [Source: HGNC Symbol; Acc: 25484] |
| 201540_at | X | q26.3 | four and a half LIM domains 1 [Source: HGNC Symbol; Acc: 3702] |
| 227325_at | 19 | q13.32 | proline rich 24 [Source: HGNC Symbol; Acc: 27406] |
| 208093_s_at | 17 | p13.1 | nudE nuclear distribution E homolog (A. nidulans)-like 1 |
| | | | [Source: HGNC Symbol; Acc: 17620] |
| 202006_at | 7 | q11.23 | protein tyrosine phosphatase, non-receptor type 12 [Source: HGNC |
| | | | Symbol; Acc: 9645] |
| 209216_at | X | p11.23 | WD repeat domain 45 [Source: HGNC Symbol; Acc: 28912] |
| 225782_at | 12 | q14.3 | methionine sulfoxide reductase B3 [Source: HGNC |
| | | | Symbol; Acc: 27375] |
| 200602_at | 21 | q21.3 | amyloid beta (A4) precursor protein [Source: HGNC |
| | | | Symbol; Acc: 620] |
| 226939_at | 4 | p15.33 | cytoplasmic polyadenylation element binding protein 2 |
| | | | [Source: HGNC Symbol; Acc: 21745] |
| 218986_s_at | 4 | q32.3 | DEAD (Asp-Glu-Ala-Asp) box polypeptide 60 [Source: HGNC |
| | | | Symbol; Acc: 25942] |
| 203139_at | 9 | q21.33 | death-asscosiated protein kinase 1 [Source: HGNC Symbol; Acc: 2674] |
| 213764_s_at | 12 | p13.31 | microfibrillar associated protein 5 [Source: HGNC |
| | | | Symbol; Acc: 29673] |
| 202565_s_at | 10 | p11.23 | supervillin [Source: HGNC Symbol; Acc: 11480] |
| 210968_s_at | 2 | p16.1 | reticulon 4 [Source: HGNC Symbol; Acc: 14085] |
| 225562_at | 13 | q34 | RAS p21 protein activator 3 [Source: HGNC Symbol; Acc: 20331] |
| 218454_at | 12 | p13.1 | phospholipase B domain containing 1 [Source: HGNC |
| | | | Symbol; Acc: 26215] |
| 209467_s_at | 1 | p33 | MAP kinase interacting serine/threonine kinase 1 [Source: HGNC |
| | | | Symbol; Acc: 7110] |
| 226869_at | 1 | p36.32 | multiple EGF-like-domains 6 [Source: HGNC Symbol; Acc: 3232] |
| 221569_at | 6 | q23.3 | Abelson helper integration site 1 [Source: HGNC Symbol; Acc: 21575] |
| 1569157_s_at | 19 | p13.2 | zinc finger protein 846 [Source: HGNC Symbol; Acc: 27260] |
| 218132_s_at | 19 | q13.42 | tRNA splicing endonuclease 34 homolog (S. cerevisiae) |
| | | | [Source: HGNC Symbol; Acc: 15506] |
| 201591_s_at | 3 | p21.1 | nischarin [Source: HGNC Symbol; Acc: 18006] |
| 227098_at | 22 | q12.2 | dual specificity phosphatase 18 [Source: HGNC Symbol; Acc: 18484] |
| 203910_at | 1 | p21.3 | Rho GTPase activating protein 29 [Source: HGNC |
| | | | Symbol; Acc: 30207] |
| 1555847_a_at | NA | NA | NA |
| 241353_s_at | NA | NA | NA |
| 202225_at | 17 | p13.3 | v-crk avian sarcoma virus CT10 oncogene homolog [Source: HGNC |
| | | | Symbol; Acc: 2362] |
| 212915_at | 3 | p13 | PDZ domain containing ring finger 3 [Source: HGNC |
| | | | Symbol; Acc: 17704] |
| 202920_at | 4 | q25 | ankyrin 2, neuronal [Source: HGNC Symbol; Acc: 493] |
| 235391_at | 8 | q22.1 | family with sequence similarity 92, member A1 [Source: HGNC |
| | | | Symbol; Acc: 30452] |
| 224906_at | 12 | q12 | anoctamin 6 [Source: HGNC Symbol; Acc: 25240] |
| 227087_at | 2 | q11.2 | inositol polyphosphate-4-phosphatase, type I, 107 kDa |
| | | | [Source: HGNC Symbol; Acc: 6074] |
| 225931_s_at | 17 | q25.3 | ring finger protein 213 [Source: HGNC Symbol; Acc: 14539] |
| 212690_at | 8 | p11.23 | DDHD domain containing 2 [Source: HGNC Symbol; Acc: 29106] |
| 209003_at | 17 | p13.2 | solute carrier family 25 (mitchondrial carrier; oxoglutarate |
| | | | carrier), member 11 [Source: HGNC Symbol; Acc: 10981] |
| 202206_at | 2 | q37.1 | ADP-ribosylation factor-like 4C [Source: HGNC Symbol; Acc: 698] |
| 232000_at | 9 | p22.3 | tetratricopeptide repeat domain 39B [Source: HGNC |
| | | | Symbol; Acc: 23704] |
| 225386_s_at | NA | NA | NA |
| 226409_at | 20 | p13 | TBC1 domain family, member 20 [Source: HGNC Symbol; Acc: 16133] |
| 204066_s_at | 2 | q37.2 | ArfGAP with GTPase domain, ankyrin repeat and PH domain 1 |
| | | | [Source: HGNC Symbol; Acc: 16922] |
| 212441_at | 4 | p16.1 | KIAA0232 [Source: HGNC Symbol; Acc: 28992] |
| 225726_s_at | 14 | q24.1 | pleckstrin homology domain containing, family H (with MyTH4 |
| | | | domain) member 1 [Source: HGNC Symbol; Acc: 17733] |
| 232094_at | 15 | q14 | katanin p80 subunit B-like 1 [Source: HGNC Symbol; Acc: 26199] |
| 225604_s_at | 9 | p13.3 | GLI pathogenesis-related 2 [Source: HGNC Symbol; Acc: 18007] |
| 205225_at | 6 | q25.1 | estrogen receptor 1 [Source: HGNC Symbol; Acc: 3467] |
| 218838_s_at | 2 | p13.1 | tetratricopeptide repeat domain 31 [Source: HGNC |
| | | | Symbol; Acc: 25759] |
| 227961_at | 8 | p23.1 | cathepsin B [Source: HGNC Symbol; Acc: 2527] |
| 1558041_a_at | 16 | q22.1 | KIAA0895-like [Source: HGNC Symbol; Acc: 34408] |
| 209540_at | 12 | q23.2 | insulin-like growth factor 1 (somatomedin C) [Source: HGNC |
| | | | Symbol; Acc: 5464] |
| 203651_at | 5 | q14.1 | zinc finger, FYVE domain containing 16 [Source: HGNC |
| | | | Symbol; Acc: 20756] |
| 224900_at | 17 | p13.2 | ankyrin repeat and FYVE domain containing 1 [Source: HGNC |
| | | | Symbol; Acc: 20763] |
| 223441_at | 6 | q13 | solute carrier family 17 (anion/sugar transporter), member 5 |
| | | | [Source: HGNC Symbol; Acc: 10933] |
| 204294_at | 3 | p21.31 | aminomethyltransferase [Source: HGNC Symbol; Acc: 473] |
| 204040_at | 2 | p25.2 | ring finger protein 144A [Source: HGNC Symbol; Acc: 20457] |
| 225272_at | 17 | p13.1 | spermidine/spermine N1-acetyltransferase family member 2 |
| | | | [Source: HGNC Symbol; Acc: 23160] |
| 204464_s_at | 4 | q31.22 | endothelin receptor type A [Source: HGNC Symbol; Acc: 3179] |
| 205011_at | 11 | q24.2 | von Willebrand factor A domain containing 5A [Source: HGNC |
| | | | Symbol; Acc: 6658] |
| 212765_at | 1 | q32.1 | calmodulin regulated spectrin-associated protein family, member 2 |
| | | | [Source: HGNC Symbol; Acc: 29188] |
| 212624_s_at | 2 | q31.1 | chimerin 1 [Source: HGNC Symbol; Acc: 1943] |
| 209213_at | 21 | q22.12 | carbonyl reductase 1 [Source: HGNC Symbol; Acc: 1548] |
| 207738_s_at | 2 | q32.1 | NCK-associated protein 1 [Source: HGNC Symbol; Acc: 7666] |
| 206856_at | 19 | q13.42 | leukocyte immunoglobulin-like receptor, subfamily B (with TM and |
| | | | ITIM domains), member 5 [Source: HGNC Symbol; Acc: 6609] |
| 200697_at | 10 | q22.1 | hexokinase 1 [Source: HGNC Symbol; Acc: 4922] |
| 219134_at | 1 | p31.1 | EGF, latrophilin and seven transmembrane domain containing 1 |
| | | | [Source: HGNC Symbol; Acc: 20822] |
| 222294_s_at | 15 | q21.3 | RAB27A, member RAS oncogene family [Source: HGNC |
| | | | Symbol; Acc: 9766] |
| 201057_s_at | 3 | q13.33 | golgin B1 [Source: HGNC Symbol; Acc: 4429] |
| 218017_s_at | 8 | p11.21 | heparan-alpha-glucosaminide N-acetyltransferase [Source: HGNC |
| | | | Symbol; Acc: 26527] |
| 226905_at | 17 | p13.3 | family with sequence similarity 101, member B [Source: HGNC |
| | | | Symbol; Acc: 28705] |
| 226616_s_at | 21 | q22.3 | NADH dehydrogenase (ubiquinone) flavoprotein 3, 10 kDa |
| | | | [Source: HGNC Symbol; Acc: 7719] |
| 215127_s_at | 2 | q24.2 | RNA binding motif, single stranded interacting protein 1 |
| | | | [Source: HGNC Symbol; Acc: 9907] |
| 212373_at | 15 | q23 | fem-1 homolog b (C. elegans) [Source: HGNC Symbol; Acc: 3649] |
| 222750_s_at | 4 | q12 | steroid 5 alpha-reductase 3 [Source: HGNC Symbol; Acc: 25812] |
| 236006_s_at | 17 | p11.2 | A kinase (PRKA) anchor protein 10 [Source: HGNC Symbol; Acc: 368] |
| 224733_at | 16 | q21 | CKLF-like MARVEL transmembrane domain containing 3 |
| | | | [Source: HGNC Symbol; Acc: 19174] |
| 204981_at | 11 | p15.4 | solute carrier family 22, member 18 [Source: HGNC |
| | | | Symbol; Acc: 10964] |
| 223077_at | 15 | q21.2 | tropomodulin 3 (ubiquitous) [Source: HGNC Symbol; Acc: 11873] |
| 209686_at | 21 | q22.3 | S100 calcium binding protein B [Source: HGNC Symbol; Acc: 10500] |
| 225325_at | 2 | q32.2 | major facilitator superfamily domain containing 6 [Source: HGNC |
| | | | Symbol; Acc: 24711] |
| 205236_x_at | 4 | p15.2 | superoxide dismutase 3, extracellular [Source: HGNC |
| | | | Symbol; Acc: 11181] |
| 228220_at | 5 | q13.2 | FCH domain only 2 [Source: HGNC Symbol; Acc: 25180] |
| 205904_at | 6 | p21.33 | MHC class I polypeptide-related sequence A [Source: HGNC |
| | | | Symbol; Acc: 7090] |
| 226777_at | 10 | q26.2 | ADAM metallopeptidase domain 12 [Source: HGNC |
| | | | Symbol; Acc: 190] |
| 213618_at | 4 | p14 | ArfGAP with RhoGAP domain, ankyrin repeat and PH domain 2 |
| | | | [Source: HGNC Symbol; Acc: 16924] |
| 205624_at | 3 | q24 | carboxypeptidase A3 (mast cell) [Source: HGNC Symbol; Acc: 2298] |
| 215268_at | 1 | p34.3 | KIAA0754 [Source: HGNC Symbol; Acc: 29111] |
| 203672_x_at | 6 | p22.3 | thiopurine S-methyltransferase [Source: HGNC Symbol; Acc: 12014] |
| 1555229_a_at | 12 | p13.31 | complement component 1, s subcomponent [Source: HGNC |
| | | | Symbol; Acc: 1247] |
| 203855_at | 1 | p13.3 | WD repeat domain 47 [Source: HGNC Symbol; Acc: 29141] |
| 207072_at | 2 | q12.1 | interleukin 18 receptor accessory protein [Source: HGNC |
| | | | Symbol; Acc: 5989] |
| 221935_s_at | 3 | p14.1 | EGF domain-specific O-linked N-acetylglucosamine (GlcNAc) |
| | | | transferase [Source: HGNC Symbol; Acc: 28526] |
| 203232_s_at | 6 | p22.3 | ataxin 1 [Source: HGNC Symbol; Acc: 10548] |
| 202096_s_at | 22 | q13.2 | translocator protein (18 kDa) [Source: HGNC Symbol; Acc: 1158] |
| 224813_at | 7 | q31.32 | Wiskott-Aldrich syndrome-like [Source: HGNC Symbol; Acc: 12735] |
| 213455_at | 4 | p14 | family with sequence similarity 114, member A1 [Source: HGNC |
| | | | Symbol; Acc: 25087] |
| 226873_at | 15 | q21.3 | family with sequence similarity 63, member B [Source: HGNC |
| | | | Symbol; Acc: 26954] |
| 1554503_a_at | 19 | q13.42 | osteoclast associated, immunoglobulin-like receptor [Source: HGNC |
| | | | Symbol; Acc: 29960] |
| 206118_at | 2 | q32.3 | signal transducer and activator of transription 4 [Source: HGNC |
| | | | Symbol; Acc: 11365] |
| 210970_s_at | 6 | q14.1 | inhibitor of Bruton agammaglobulinemia tyrosine kinase |
| | | | [Source: HGNC Symbol; Acc: 17853] |
| 227113_at | 8 | q13.1 | alcohol dehydrogenase, iron containing, 1 [Source: HGNC |
| | | | Symbol; Acc: 16354] |
| 202949_s_at | 2 | q12.2 | four and a half LIM domains 2 [Source: HGNC Symbol; Acc: 3703] |
| 225579_at | 2 | p25.1 | PQ loop repeat containing 3 [Source: HGNC Symbol; Acc: 28503] |
| 235291_s_at | NA | NA | NA |
| 203939_at | 6 | q14.3 | 5′-nucleotidase, ecto (CD73) [Source: HGNC Symbol; Acc: 8021] |
| 214274_s_at | 3 | p22.2 | acetyl-CoA acyltransferase 1 [Source: HGNC Symbol; Acc: 82] |
| 204046_at | 15 | q15.1 | phospholipase C, beta 2 [Source: HGNC Symbol; Acc: 9055] |
| 202392_s_at | 22 | q12.2 | phosphatidylserine decarboxylase [Source: HGNC Symbol; Acc: 8999] |
| 218326_s_at | 11 | p14.1 | leucine-rich repeat containing G protein-coupled receptor 4 |
| | | | [Source: HGNC Symbol; Acc: 13299] |
| 229119_s_at | 17 | p12 | zinc finger, SWIM-type containing 7 [Source: HGNC |
| | | | Symbol; Acc: 26993] |
| 206491_s_at | 19 | q13.33 | N-ethylmaleimide-sensitive factor attachment protein, alpha |
| | | | [Source: HGNC Symbol; Acc: 7641] |
| 227450_at | 12 | p12.3 | endoplasmic reticulum protein 27 [Source: HGNC |
| | | | Symbol; Acc: 26495] |
| 200866_s_at | 10 | q22.1 | prosaposin [Source: HGNC Symbol; Acc: 9498] |
| 200720_s_at | 10 | q24.32 | ARP1 actin-related protein 1 homolog A, centractin alpha (yeast) |
| | | | [Source: HGNC Symbol; Acc: 167] |
| 232064_at | 5 | q21.3 | fer (fps/fes related) tyrosine kinase [Source: HGNC |
| | | | Symbol; Acc: 3655] |
| 217922_at | 1 | p12 | mannosidase, alpha, class 1A, member 2 [Source: HGNC |
| | | | Symbol; Acc: 6822] |
| 201944_at | 5 | q13.3 | hexosaminidase B (beta polypeptide) [Source: HGNC |
| | | | Symbol; Acc: 4879] |
| 210986_s_at | 15 | q22.2 | tropomyosin 1 (alpha) [Source: HGNC Symbol; Acc: 12010] |
| 227568_at | 10 | q23.32 | HECT domain containing E3 ubiquitin protein ligase 2 |
| | | | [Source: HGNC Symbol; Acc: 26736] |
| 200645_at | 17 | p13.1 | GABA(A) receptor-associated protein [Source: HGNC |
| | | | Symbol; Acc: 4067] |
| 219892_at | 15 | q25.2 | transmembrane 6 superfamily member 1 [Source: HGNC |
| | | | Symbol; Acc: 11860] |
| 222431_at | 9 | q22.1 | spindlin 1 [Source: HGNC Symbol; Acc: 11243] |
| 203665_at | 22 | q12.3 | heme oxygenase (decycling) 1 [Source: HGNC Symbol; Acc: 5013] |
| 202506_at | 2 | q31.3 | sperm specific antigen 2 [Source: HGNC Symbol; Acc: 11319] |
| 221899_at | 13 | q13.1 | NEDD4 binding protein 2-like 2 [Source: HGNC Symbol; Acc: 26916] |
| 203668_at | 15 | q24.2 | mannosidase, alpha, class 2C, member 1 [Source: HGNC |
| | | | Symbol; Acc: 6827] |
| 206295_at | 11 | q23.1 | interleukin 18 (interferon-gamma-inducing factor) [Source: HGNC |
| | | | Symbol; Acc: 5986] |
| 228152_s_at | 4 | q32.3 | DEAD (Asp-Glu-Ala-Asp) box polypeptide 60-like [Source: HGNC |
| | | | Symbol; Acc: 26429] |
| 228341_at | 3 | q22.1 | nudix (nucleoside diphosphate linked moeity X)-type motif 16 |
| | | | [Source: HGNC Symbol; Acc: 26442] |
| 218164_at | 17 | q21.33 | spermatogenesis associated 20 [Source: HGNC Symbol; Acc: 26125] |
| 227070_at | 12 | q23.3 | glycosyltransferase 8 domain containing 2 [Source: HGNC |
| | | | Symbol; Acc: 24890] |
| 213083_at | 9 | q22.32 | solute carrier family 35 (UDP-GlcNAc/UDP-glucose transporter), |
| | | | member D2 [Source: HGNC Symbol; Acc: 20799] |
| 224772_at | 1 | q32.1 | neuron navigator 1 [Source: HGNC Symbol; Acc: 15989] |
| 229287_at | 14 | q24.2 | pecanex homolog (Drosophila) [Source: HGNC Symbol; Acc: 19740] |
| 201200_at | 1 | q24.2 | cellular repressor of E1A-stimulated genes 1 [Source: HGNC |
| | | | Symbol; Acc: 2351] |
| 229450_at | 10 | q23.31 | interferon-induced protein with tetratricopeptide repeats 3 |
| | | | [Source: HGNC Symbol; Acc: 5411] |
| 228249_at | 11 | p12 | chromosome 11 open reading frame 74 [Source: HGNC |
| | | | Symbol; Acc: 25142] |
| 38487_at | 3 | p21.1 | stabilin 1 [Source: HGNC Symbol; Acc: 18628] |
| 202974_at | X | q28 | membrane protein, palmitoylated 1, 55 kDa [Source: HGNC |
| | | | Symbol; Acc: 7219] |
| 226510_at | 14 | q12 | HEAT repeat containing 5A [Source: HGNC Symbol; Acc: 20276] |
| 228282_at | 4 | q28.2 | major facilitator superfamily domain containing 8 [Source: HGNC |
| | | | Symbol; Acc: 28486] |
| 203675_at | 11 | p15.1 | nucleobindin 2 [Source: HGNC Symbol; Acc: 8044] |
| 228384_s_at | 10 | q24.2 | pyridine nucleotide-disulphide oxidoreductase domain 2 |
| | | | [Source: HGNC Symbol; Acc: 23517] |
| 229367_s_at | 7 | q36.1 | GTPase, IMAP family member 6 [Source: HGNC Symbol; Acc: 21918] |
| 218309_at | 1 | p36.12 | calcium/calmodulin-dependent protein kinase II inhibitor 1 |
| | | | [Source: HGNC Symbol; Acc: 24190] |
| 34726_at | 12 | q13.12 | calcium channel, voltage-dependent, beta 3 subunit [Source: HGNC |
| | | | Symbol; Acc: 1403] |
| 212670_at | 7 | q11.23 | elastin [Source: HGNC Symbol; Acc: 3327] |
| 229800_at | 13 | q13.3 | doublecortin-like kinase 1 [Source: HGNC Symbol; Acc: 2700] |
| 201975_at | 12 | q24.31 | CAP-GLY domain containing linker protein 1 [Source: HGNC |
| | | | Symbol; Acc: 10461] |
| 224413_s_at | 8 | p11.22 | TM2 domain containing 2 [Source: HGNC Symbol; Acc: 24127] |
| 204011_at | 13 | q31.1 | sprouty homolog 2 (Drosophila) [Source: HGNC Symbol; Acc: 11270] |
| 235033_at | 20 | q13.32 | aminopeptidase-like 1 [Source: HGNC Symbol; Acc: 16244] |
| 228348_at | 15 | q26.3 | lines homolog (Drosophila) [Source: HGNC Symbol; Acc: 30922] |
| 227628_at | 5 | q11.2 | glutathione peroxidase 8 (putative) [Source: HGNC |
| | | | Symbol; Acc: 33100] |
| 1557112_a_at | 17 | p13.3 | vacuolar protein sorting 53 homolog (S. cerevisiae) [Source: HGNC |
| | | | Symbol; Acc: 25608] |
| 228071_at | 7 | q36.1 | GTPase, IMAP family member 7 [Source: HGNC Symbol; Acc: 22404] |
| 209264_s_at | 11 | p15.5 | tetraspanin 4 [Source: HGNC Symbol; Acc: 11859] |
| 208892_s_at | 12 | q21.33 | dual specificity phophatase 6 [Source: HGNC Symbol; Acc: 3072] |
| 201315_x_at | 11 | p15.5 | interferon induced transmembrane protein 2 [Source: HGNC |
| | | | Symbol; Acc: 5413] |
| 222802_at | 6 | p24.1 | endothelin 1 [Source: HGNC Symbol; Acc: 3176] |
| 1553395_a_at | 3 | q13.2 | CD200 receptor 1 [Source: HGNC Symbol; Acc: 24235] |
| 226399_at | 4 | q23 | DnaJ (Hsp40) homolog, subfamily B, member 14 [Source: HGNC |
| | | | Symbol; Acc: 25881] |
| 226490_at | 6 | q24.1 | NHS-like 1 [Source: HGNC Symbol; Acc: 21021] |
| 222513_s_at | 10 | q24.1 | sorbin and SH3 domain containing 1 [Source: HGNC |
| | | | Symbol; Acc: 14565] |
| 218705_s_at | 5 | q23.2 | sorting nexin 24 [Source: HGNC Symbol; Acc: 21533] |
| 226837_at | 15 | q14 | sprouty-related, EVH1 domain containing 1 [Source: HGNC |
| | | | Symbol; Acc: 20249] |
| 225338_at | 1 | p32.3 | zyg-11 family member B, cell cycle regulator [Source: HGNC |
| | | | Symbol; Acc: 25820] |
| 204955_at | X | p11.4 | sushi-repeat containing protein, X-linked [Source: HGNC |
| | | | Symbol; Acc: 11309] |
| 210840_s_at | 15 | q26.1 | IQ motif containing GTPase activating protein 1 [Source: HGNC |
| | | | Symbol; Acc: 6110] |
| 201494_at | 11 | q14.1 | prolylcarboxypeptidase (angiotensinase C) [Source: HGNC |
| | | | Symbol; Acc: 9344] |
| 204415_at | 1 | p36.11 | interferon, alpha-inductible protein 6 [Source: HGNC |
| | | | Symbol; Acc: 4054] |
| 209290_s_at | 9 | p22.3 | nuclear factor I/B [Source: HGNC Symbol; Acc: 7785] |
| 221474_at | 18 | p11.31 | myosin, light chain 12B, regulatory [Source: HGNC |
| | | | Symbol; Acc: 29827] |
| 235747_at | 10 | q21.3 | solute carrier family 25 (mitochondrial carrier; Graves disease |
| | | | autoantigen), member 16 [Source: HGNC Symbol; Acc: 10986] |
| 201924_at | 4 | q21.3 | AF4/FMR2 family, member 1 [Source: HGNC Symbol; Acc: 7135] |
| 218045_x_at | 12 | p13.31 | parathymosin [Source: HGNC Symbol; Acc: 9629] |
| 213943_at | 7 | p21.1 | twist basic helix-loop-helix transcription factor 1 [Source: HGNC |
| | | | Symbol; Acc: 12428] |
| 226752_at | 5 | q21.1 | Family with sequence similarity 174, member A [Source: HGNC |
| | | | Symbol; Acc: 24943] |
| 201876_at | 7 | q21.3 | paraoxonase 2 [Source: HGNC Symbol; Acc: 9205] |
| 1554240_a_at | 16 | p11.2 | integrin, alpha L (antigen CD11A (p180), lymphocyte function- |
| | | | associated antigen 1; alpha polypeptide) [Source: HGNC |
| | | | Symbol; Acc: 6148] |
| 203989_x_at | 5 | q13.3 | coagulation factor II (thrombin) receptor [Source: HGNC |
| | | | Symbol; Acc: 3537] |
| 226425_at | 2 | p23.2 | CAP-GLY domain containing linker protein family, member 4 |
| | | | [Source: HGNC Symbol; Acc: 26108] |
| 200762_at | 8 | p21.2 | dihydropyrimidinase-like 2 [Source: HGNC Symbol; Acc: 3014] |
| 223681_s_at | 1 | p31.3 | InaD-like (Drosophila) [Source: HGNC Symbol; Acc: 28881] |
| 211986_at | 11 | q12.3 | AHNAK nucleoprotein [Source: HGNC Symbol; Acc: 347] |
| 204112_s_at | 2 | q22.1 | histamine N-methyltransferase [Source: HGNC Symbol; Acc: 5028] |
| 235360_at | 2 | q33.3 | pleckstrin homology domain containing, family M, member 3 |
| | | | [Source: HGNC Symbol; Acc: 34006] |
| 209276_s_at | 5 | q15 | glutaredoxin (thioltransferase) [Source: HGNC Symbol; Acc: 4330] |
| 226113_at | 1 | p36.12 | zinc finger protein 436 [Source: HGNC Symbol; Acc: 20814] |
| 201694_at | 5 | q31.2 | early growth response 1 [Source: HGNC Symbol; Acc: 3238] |
| 204417_at | 14 | q31.3 | galactosylceramidase [Source: HGNC Symbol; Acc: 4115] |
| 211178_s_at | 15 | q24.3 | proline-serine-threonine phosphatase interacting protein 1 |
| | | | [Source: HGNC Symbol; Acc: 9580] |
| 203088_at | 14 | q32.12 | fibulin 5 [Source: HGNC Symbol; Acc: 3602] |
| 218450_at | 12 | p13.1 | heme binding protein 1 [Source: HGNC Symbol; Acc: 17176] |
| 210113_s_at | 17 | p13.2 | NLR family, pyrin domain containing 1 [Source: HGNC |
| | | | Symbol; Acc: 14374] |
| 225673_at | 19 | q13.42 | myeloid-associated differentiation marker [Source: HGNC |
| | | | Symbol; Acc: 7544] |
| 31874_at | 22 | q12.2 | growth arrest-specific 2 like 1 [Source: HGNC Symbol; Acc: 16955] |
| 222597_at | 22 | q11.21 | synaptosomal-associated protein, 29 kDa [Source: HGNC |
| | | | Symbol; Acc: 11133] |
| 226279_at | 11 | q14.2 | protease, serine, 23 [Source: HGNC Symbol; Acc: 14370] |
| 235570_at | 3 | p24.1 | RNA binding motif, single stranded interacting protein 3 |
| | | | [Source: HGNC Symbol; Acc: 13427] |
| 214830_at | 14 | q23.1 | solute carrier family 38, member 6 [Source: HGNC |
| | | | Symbol; Acc: 19863] |
| 201366_at | 10 | q22.2 | annexin A7 [Source: HGNC Symbol; Acc: 545] |
| 203179_at | 9 | p13.3 | galactose-1-phosphate uridylyltransferase [Source: HGNC |
| | | | Symbol; Acc: 4135] |
| 225919_s_at | 9 | p21.2 | chromosome 9 open reading frame 72 [Source: HGNC |
| | | | Symbol; Acc: 28337] |
| 36711_at | 22 | q13.1 | v-maf avian musculoaponeurotic fibrosarcoma oncogene homolog |
| | | | F [Source: HGNC Symbol; Acc: 6780] |
| 205174_s_at | 2 | p22.2 | glutaminyl-peptide cyclotransferase [Source: HGNC |
| | | | Symbol; Acc: 9753] |
| 225032_at | 3 | q26.31 | fibronectin type III domain containing 3B [Source: HGNC |
| | | | Symbol; Acc: 24670] |
| 225283_at | 15 | q26.2 | arrestin domain containing 4 [Source: HGNC Symbol; Acc: 28087] |
| 212820_at | 15 | q21.2 | Dmx-like 2 [Source: HGNC Symbol; Acc: 2938] |
| 202411_at | 14 | q32.12 | interferon, alpha-inducible protein 27 [Source: HGNC |
| | | | Symbol; Acc: 5397] |
| 201444_s_at | X | p11.4 | ATPase, H transporting, lysosomal accessory protein 2 |
| | | | [Source: HGNC Symbol; Acc: 18305] |
| 212209_at | 12 | q24.21 | mediator complex subunit 13-like [Source: HGNC |
| | | | Symbol; Acc: 22962] |
| 228082_at | 11 | q24.1 | CXADR-like membrane protein [Source: HGNC Symbol; Acc: 24039] |
| 201579_at | 4 | q35.2 | FAT atypical cadherin 1 [Source: HGNC Symbol; Acc: 3595] |
| 201050_at | 19 | q13.2 | phospholipase D family, member 3 [Source: HGNC |
| | | | Symbol; Acc: 17158] |
| 205726_at | X | q21.33 | diaphanous-related formin 2 [Source: HGNC Symbol; Acc: 2877] |
| 212717_at | 17 | q21.31 | pleckstrin homology domain containing, family M (with RUN |
| | | | domain) member 1 [Source: HGNC Symbol; Acc: 29017] |
| 202827_s_at | 14 | q11.2 | matrix metallopeptidase 14 (membrane-inserted) [Source: HGNC |
| | | | Symbol; Acc: 7160] |
| 201594_s_at | 18 | p11.22 | protein phosphatase 4, regulatory subunit 1 [Source: HGNC |
| | | | Symbol; Acc: 9320] |
| 203460_s_at | 14 | q24.2 | presenilin 1 [Source: HGNC Symbol; Acc: 9508] |
| 221840_at | 10 | q26.2 | protein tyrosine phosphatase, receptor type, E [Source: HGNC |
| | | | Symbol; Acc: 9669] |
| 203410_at | 8 | p11.21 | adaptor-related protein complex 3, mu 2 subunit [Source: HGNC |
| | | | Symbol; Acc: 570] |
| 226582_at | NA | NA | NA |
| 209600_s_at | 17 | q25.1 | acyl-CoA oxidase 1, palmitoyl [Source: HGNC Symbol; Acc: 119] |
| 221814_at | 8 | p11.23 | G protein-coupled receptor 124 [Source: HGNC Symbol; Acc: 17849] |
| 230836_at | 5 | q21.1 | ST8 alpha-N-acetyl-neuraminide alpha-2,8-sialyltransferase 4 |
| | | | [Source: HGNC Symbol; Acc: 10871] |
| 204270_at | 1 | p36.33 | v-ski avian sarcoma viral oncogene homolog [Source: HGNC |
| | | | Symbol; Acc: 10896] |
| 200872_at | 1 | q21.3 | S100 calcium binding protein A10 [Source: HGNC |
| | | | Symbol; Acc: 10487] |
| 219761_at | 12 | p13.2 | C-type lectin domain family 1, member 1, member A [Source: HGNC |
| | | | Symbol; Acc: 24355] |
| 212708_at | 17 | q21.1 | male-specific lethal 1 homolog (Drosophila) [Source: HGNC |
| | | | Symbol; Acc: 27905] |
| 204326_x_at | 16 | q13 | metallothionein 1X [Source: HGNC Symbol; Acc: 7405] |
| 241392_at | 3 | q13.33 | transmembrane protein 39A [Source: HGNC Symbol; Acc: 25600] |
| 205771_s_at | 6 | q23.2 | A kinase (PRKA) anchor protein 7 [Source: HGNC Symbol; Acc: 377] |
| 206227_at | 15 | q22.31 | cartilage intermediate layer protein, nucleotide |
| | | | pyrophosphohydrolase [Source: HGNC Symbol; Acc: 1980] |
| 204158_s_at | 11 | q13.2 | T-cell, immune regulator 1, ATPase, H Transporting, lysosomal V0 |
| | | | subunit A3 [Source: HGNC Symbol; Acc: 11647] |
| 212948_at | 17 | p13.2 | calmodulin binding transcription activator 2 [Source: HGNC |
| | | | Symbol; Acc: 18807] |
| 218241_at | 14 | q32.12 | golgin A5 [Source: HGNC Symbol; Acc: 4428] |
| 203042_at | X | q24 | lysosomal-associated membrane protein 2 [Source: HGNC |
| | | | Symbol; Acc: 6501] |
| 223264_at | 15 | q25.1 | mesoderm development candidate 1 [Source: HGNC |
| | | | Symbol; Acc: 13519] |
| 243141_at | 4 | q25 | sphingomyelin synthase 2 [Source: HGNC Symbol; Acc: 28395] |
| 212513_s_at | 1 | p31.1 | ubiquitin specific peptidase 33 [Source: HGNC Symbol; Acc: 20059] |
| 44111_at | 15 | q26.1 | vacuolar protein sorting 33 homolog B (yeast) [Source: HGNC |
| | | | Symbol; Acc: 12712] |
| 203044_at | 15 | q26.3 | chondroitin sulfate synthase 1 [Source: HGNC Symbol; Acc: 17198] |
| 201133_s_at | 5 | q21.3 | praja ring finger 2, E3 ubiquitin protein ligase [Source: HGNC |
| | | | Symbol; Acc: 17481] |
| 238478_at | 9 | p22.2 | basonuclin 2 [Source: HGNC Symbol; Acc: 30988] |
| 207121_s_at | 15 | q21.2 | mitogen-activated protein kinase 6 [Source: HGNC |
| | | | Symbol; Acc: 3879] |
| 226757_at | 10 | q23.31 | interferon-induced protein with tetratricopeptide repeats 2 |
| | | | [Source: HGNC Symbol; Acc: 5409] |
| 224929_at | 5 | q31.2 | transmembrane protein 173 [Source: HGNC Symbol; Acc: 27962] |
| 219013_at | 7 | q36.1 | UDP-N-acetyl-alpha-D-galactosamine:polypeptide N- |
| | | | acetylgalactosaminyltransferase 11 (GalNAc-T11) [Source: HGNC |
| | | | Symbol; Acc: 19875] |
| 203732_at | 15 | q22.31 | thyroid hormone receptor interactor 4 [Source: HGNC |
| | | | Symbol; Acc: 12310] |
| 206991_s_at | 3 | p21.31 | chemokine (C-C motif) receptor 5 (gene/pseudogene) |
| | | | [Source: HGNC Symbol; Acc: 1606] |
| 212192_at | 13 | q22.3 | potassium channel tetramerization domain containing 12 |
| | | | [Source: HGNC Symbol; Acc: 14678] |
| 212071_s_at | 2 | p16.2 | spectrin, beta, non-erythrocytic 1 [Source: HGNC |
| | | | Symbol; Acc: 11275] |
| 204194_at | 21 | q21.3 | BTB and CNC homology 1, basic leucine zipper transcription factor |
| | | | 1 [Source: HGNC Symbol; Acc: 935] |
| 213469_at | 2 | q33.1 | post-GPI attachment to proteins 1 [Source: HGNC |
| | | | Symbol; Acc: 25712] |
| 207173_x_at | 16 | q21 | cadherin 11, type 2, OB-cadherin (osteoblast) [Source: HGNC |
| | | | Symbol; Acc: 1750] |
| 202020_s_at | 2 | q34 | LanC lantibiotic synthetase component C-like 1 (bacterial) |
| | | | [Source: HGNC Symbol; Acc: 6508] |
| 201384_s_at | 17 | q21.31 | neighbor of BRCA1 gene 1 [Source: HGNC Symbol; Acc: 6746] |
| 213004_at | 9 | q33.3 | angiopoietin-like 2 [Source: HGNC Symbol; Acc: 490] |
| 203310_at | 1 | p13.3 | syntaxin binding protein 3 [Source: HGNC Symbol; Acc: 11446] |
| 212239_at | 5 | q13.1 | phosphoinositide-3-kinase, regulatory subunit 1 (alpha) |
| | | | [Source: HGNC Symbol; Acc: 8979] |
| 225864_at | 8 | q24.21 | family with sequence similarity 84, member B [Source: HGNC |
| | | | Symbol; Acc: 24166] |
| 218679_s_at | 8 | q24.3 | vacuolar protein sorting 28 homolog (S. cerevisiae) [Source: HGNC |
| | | | Symbol; Acc: 18178] |
| 211964_at | 13 | q34 | collagen, type IV, alpha 2 [Source: HGNC Symbol; Acc: 2203] |
| 212501_at | 20 | q13.13 | CCAAT/enhancer binding protein (C/EBP), beta [Source: HGNC |
| | | | Symbol; Acc: 1834] |
| 215596_s_at | 21 | q21.3 | listerin E3 ubiquitin protein ligase 1 [Source: HGNC |
| | | | Symbol; Acc: 13082] |
| 235306_at | 7 | q36.1 | GTPase, IMAP family member 8 [Source: HGNC Symbol; Acc: 21792] |
| 213746_s_at | X | q28 | filamin A, alpha [Source: HGNC Symbol; Acc: 3754] |
| 200982_s_at | 5 | q33.1 | annexin A6 [Source: HGNC Symbol; Acc: 544] |
| 227029_at | 14 | q13.2 | family with sequence similarity 177, member A1 [Source: HGNC |
| | | | Symbol; Acc: 19829] |
| 225695_at | 2 | p23.3 | solute carrier family 35, member F6 [Source: HGNC |
| | | | Symbol; Acc: 26055] |
| 230263_s_at | 8 | p21.2 | dedicator of cytokinesis 5 [Source: HGNC Symbol; Acc: 23476] |
| 219860_at | 6 | p21.33 | lymphocyte antigen 6 complex, locus G5C [Source: HGNC |
| | | | Symbol; Acc: 13932] |
| 216620_s_at | 8 | p23.3 | Rho guanine nucleotide exchange factor (GEF) 10 [Source: HGNC |
| | | | Symbol; Acc: 14103] |
| 209298_s_at | 21 | q22.11 | intersectin 1 (SH3 domain protein) [Source: HGNC |
| | | | Symbol; Acc: 6183] |
| 219383_at | 11 | p13 | proline rich 5 like [Source: HGNC Symbol; Acc: 25878] |
| 218204_s_at | 3 | p21.31 | FYVE and coiled-coil domain containing 1 [Source: HGNC |
| | | | Symbol; Acc: 14673] |
| 212637_s_at | 8 | q21.3 | WW domain containing E3 ubiquitin protein ligase 1 [Source: HGNC |
| | | | Symbol; Acc: 17004] |
| 200784_s_at | 12 | q13.3 | low density lipoprotein receptor-related protein 1 [Source: HGNC |
| | | | Symbol; Acc: 6692] |
| 202668_at | 13 | q33.3 | ephrin-B2 [Source: HGNC Symbol; Acc: 3227] |
| 211926_s_at | 22 | q12.3 | myosin, heavy chain 9, non-muscle [Source: HGNC |
| | | | Symbol; Acc: 7579] |
| 207181_s_at | 10 | q25.3 | caspase 7, apoptosis-related cysteine peptidase [Source: HGNC |
| | | | Symbol; Acc: 1508] |
| 203940_s_at | 14 | q24.3 | vasohibin 1 [Source: HGNC Symbol; Acc: 19964] |
| 225046_at | NA | NA | NA |
| 229699_at | NA | NA | NA |
| 1555997_s_at | 2 | q35 | insulin-like growth factor binding protein 5 [Source: HGNC |
| | | | Symbol; Acc: 5474] |
| 221858_at | 10 | q23.33 | TBC1 domain family, member 12 [Source: HGNC Symbol; Acc: 29082] |
| 202123_s_at | 9 | q34.12 | c-abl oncogene 1, non-receptor tyrosine kinase [Source: HGNC |
| | | | Symbol; Acc: 76] |
| 209189_at | 14 | q24.3 | FBJ murine osteosarcoma viral oncogene homolog [Source: HGNC |
| | | | Symbol; Acc: 3796] |
| 231697_s_at | 17 | q23.1 | vacuole membrane protein 1 [Source: HGNC Symbol; Acc: 29559] |
| 231823_s_at | 5 | q35.1 | SH3 and PX domains 2B [Source: HGNC Symbol; Acc: 29242] |
| 226917_s_at | 4 | p15.2 | anaphase promoting complex subunit 4 [Source: HGNC |
| | | | Symbol; Acc: 19990] |
| 213135_at | 21 | q22.11 | T-cell lymphoma invasion and metastasis 1 [Source: HGNC |
| | | | Symbol; Acc: 11805] |
| 222793_at | 9 | p21.1 | DEAD (Asp-Glu-Ala-Asp) box polypeptide 58 [Source: HGNC |
| | | | Symbol; Acc: 19102] |
| 235059_at | 18 | p11.22 | RAB12, member RAS oncogene family [Source: HGNC |
| | | | Symbol; Acc: 31332] |
| 213364_s_at | 15 | q22.31 | sorting nexin 1 [Source: HGNC Symbol; Acc: 11172] |
| 213194_at | 3 | p12.2 | roundabout, axon guidance receptor, homolog 1 (Drosophila) |
| | | | [Source: HGNC Symbol; Acc: 10249] |
| 223434_at | 1 | p22.2 | guanylate binding protein 3 [Source: HGNC Symbol; Acc: 4184] |
| 201464_x_at | 1 | p32.1 | jun proto-oncogene [Source: HGNC Symbol; Acc: 6204] |
| 228325_at | NA | NA | NA |
| 226639_at | 2 | q14.3 | SFT2 domain containing 3 [Source: HGNC Symbol; Acc: 28767] |
| 204204_at | 9 | q32 | solute carrier family 31 (copper transporter), member 2 |
| | | | [Source: HGNC Symbol; Acc: 11017] |
| 213659_at | X | q26.3 | zinc finger protein 75D [Source: HGNC Symbol; Acc: 13145] |
| 219165_at | 8 | p21.3 | PDZ and LIM domain 2 (mystique) [Source: HGNC |
| | | | Symbol; Acc: 13992] |
| 202704_at | 17 | q21.33 | transducer of ERBB2, 1 [Source: HGNC Symbol; Acc: 11979] |
| 201059_at | 11 | q13.3 | cortacin [Source: HGNC Symbol; Acc: 3338] |
| 208626_s_at | 17 | q21.31 | vesicle amine transport protein 1 homolog (T. californica) |
| | | | [Source: HGNC Symbol; Acc: 16919] |
| 224285_at | X | q21.1 | G protein-coupled receptor 174 [Source: HGNC Symbol; Acc: 30245] |
| 202746_at | X | q21.1 | integral membrane protein 2A [Source: HGNC Symbol; Acc: 6173] |
| 1557749_at | 11 | q13.1 | EH domain binding protein 1-like 1 [Source: HGNC |
| | | | Symbol; Acc: 30682] |
| 208671_at | 6 | q22.31 | serine incorporator 1 [Source: HGNC Symbol; Acc: 13464] |
| 235199_at | 18 | q12.1 | ring finger protein 125, E3 ubiquitin protein ligase [Source: HGNC |
| | | | Symbol; Acc: 21150] |
| 236565_s_at | 15 | q23 | La ribonucleoprotein domain family, member 6 [Source: HGNC |
| | | | Symbol; Acc: 24012] |
| 204575_s_at | 12 | q13.2 | matrix metallopeptidase 19 [Source: HGNC Symbol; Acc: 7165] |
| 221653_x_at | 22 | q12.3 | apolipoprotein L, 2 [Source: HGNC Symbol; Acc: 619] |
| 224804_s_at | 15 | q24.1 | family with sequence similarity 219, member B [Source: HGNC |
| | | | Symbol; Acc: 24695] |
| 202820_at | 7 | p21.1 | aryl hydrocarbon receptor [Source: HGNC Symbol; Acc: 348] |
| 204082_at | 9 | q33.3 | pre-B-cell leukemia homeobox 3 [Source: HGNC Symbol; Acc: 8634] |
| 218109_s_at | 3 | q25.32 | major facilitator superfamily domain containing 1 [Source: HGNC |
| | | | Symbol; Acc: 25874] |
| 231897_at | 9 | q31.3 | prostaglandin reductase 1 [Source: HGNC Symbol; Acc: 18429] |
| 205786_s_at | 16 | p11.2 | integrin, alpha M (complement component 3 receptor 3 subunit) |
| | | | [Source: HGNC Symbol; Acc: 6149] |
| 203510_at | 7 | q31.2 | met proto-oncogene [Source: HGNC Symbol; Acc: 7029] |
| 224896_s_at | 2 | q13 | tubulin tyrosine ligase [Source: HGNC Symbol; Acc: 21586] |
| 232645_at | NA | NA | NA |
| 226576_at | 5 | q31.3 | Rho GTPase activating protein 26 [Source: HGNC |
| | | | Symbol; Acc: 17073] |
| 219191_s_at | 12 | q13.13 | bridging integrator 2 [Source: HGNC Symbol; Acc: 1053] |
| 214853_s_at | 1 | q21.3 | SHC (Src homology 2 domain containing) transforming protein 1 |
| | | | [Source: HGNC Symbol; Acc: 10840] |
| 224358_s_at | 11 | q12.2 | membrane-spanning 4-domains, subfamily A, member 7 |
| | | | [Source: HGNC Symbol; Acc: 13378] |
| 209164_s_at | 17 | q23.3 | cytochrome b561 [Source: HGNC Symbol; Acc: 2571] |
| 222175_s_at | 22 | q11.21 | mediator complex subunit 15 [Source: HGNC Symbol; Acc: 14248] |
| 219469_at | 11 | q22.3 | dynein, cytoplasmic 2, heavy chain 1 [Source: HGNC |
| | | | Symbol; Acc: 2962] |
| 226143_at | 17 | p11.2 | retinoic acid induced 1 [Source: HGNC Symbol; Acc: 9834] |
| 212681_at | 18 | p11.31 | erythrocyte membrane protein band 4.1-like 3 [Source: HGNC |
| | | | Symbol; Acc: 3380] |
| 215784_at | 1 | q23.1 | CD1e molecule [Source: HGNC Symbol; Acc: 1638] |
| 211161_s_at | 2 | q32.2 | collagen, type III, alpha 1 [Source: HGNC Symbol; Acc: 2201] |
| 209906_at | 12 | p13.31 | complement component 3a receptor 1 [Source: HGNC |
| | | | Symbol; Acc: 1319] |
| 200625_s_at | 1 | p34.2 | CAP, adenylate cyclase-associated protein 1 (yeast) [Source: HGNC |
| | | | Symbol; Acc: 20040] |
| 209550_at | 15 | q11.2 | necdin, melanoma antigen (MAGE) family member [Source: HGNC |
| | | | Symbol; Acc: 7675] |
| 214039_s_at | 8 | q22.1 | lysosomal protein transmembrane 4 beta [Source: HGNC |
| | | | Symbol; Acc: 13646] |
| 209356_x_at | 11 | q13.1 | EGF containing fibulin-like extracellular matrix protein 2 |
| | | | [Source: HGNC Symbol; Acc: 3219] |
| 207276_at | X | q27.1 | cerebellar degeneration-related protein 1, 34 kDa [Source: HGNC |
| | | | Symbol; Acc: 1798] |
| 209955_s_at | 2 | q24.2 | fibroblast activation protein, alpha [Source: HGNC |
| | | | Symbol; Acc: 3590] |
| 226844_at | 9 | p21.2 | MOB kinase activator 3B [Source: HGNC Symbol; Acc: 23825] |
| 222468_at | 1 | p34.3 | KIAA0319-like [Source: HGNC Symbol; Acc: 30071] |
| 226056_at | 3 | q13.33 | Rho GTPase activating protein 31 [Source: HGNC |
| | | | Symbol; Acc: 29216] |
| 226695_at | 1 | q24.2 | paired related homeobox 1 [Source: HGNC Symbol; Acc: 9142] |
| 204844_at | 4 | q25 | glutamyl aminopeptidase (aminopeptidase A) [Source: HGNC |
| | | | Symbol; Acc: 3355] |
| 200612_s_at | 17 | q12 | adaptor-related protein complex 2, beta 1 subunit [Source: HGNC |
| | | | Symbol; Acc: 563] |
| 200923_at | 17 | q25.3 | lectin, galactoside-binding soluble, 3 binding protein |
| | | | [Source: HGNC Symbol; Acc: 6564] |
| 208074_s_at | 19 | q13.32 | adaptor-related protein complex 2, sigma 1 subunit [Source: HGNC |
| | | | Symbol; Acc: 565] |
| 200897_s_at | 4 | q32.3 | palladin, cytoskeletal associated protein [Source: HGNC |
| | | | Symbol; Acc: 17068] |
| 209667_at | 16 | q22.1 | carboxylesterase 2 [Source: HGNC Symbol; Acc: 1864] |
| 225785_at | 10 | q21.3 | receptor accessory protein 3 [Source: HGNC Symbol; Acc: 23711] |
| 227265_at | 7 | q11.23 | fibrinogen-like 2 [Source: HGNC Symbol; Acc: 396] |
| 226679_at | 17 | q25.3 | solute carrier family 26, member 11 [Source: HGNC |
| | | | Symbol; Acc: 14471] |
| 202441_at | 10 | q24.31 | ER lipid raft associated 1 [Source: HGNC Symbol; Acc: 16947] |
| 227758_at | 12 | q12.3 | RAS-like, estrogen-related, growth inhibitor [Source: HGNC |
| | | | Symbol; Acc: 15980] |
| 204036_at | 9 | q31.3 | lysophosphatidic acid receptor 1 [Source: HGNC Symbol; Acc: 3166] |
| 225755_at | 3 | p21.31 | kelch domain containing 8B [Source: HGNC Symbol; Acc: 28557] |
| 209571_at | 2 | q31.1 | corepressor interacting with RBPJ, 1 [Source: HGNC |
| | | | Symbol; Acc: 24217] |
| 210184_at | 16 | p11.2 | integrin, alpha X (complement component 3 receptor 4 subunit) |
| | | | [Source: HGNC Symbol; Acc: 6152] |
| 217940_s_at | 13 | q34 | carbohydrate kinase domain containing [Source: HGNC |
| | | | Symbol; Acc: 25576] |
| 212993_at | 9 | q34.3 | NACC family member 2, BEN and BTB (POZ) domain containing |
| | | | [Source: HGNC Symbol; Acc: 23846] |
| 1562876_s_at | NA | NA | NA |
| 242268_at | 10 | p14 | CUGBP, Elav-like family member 2 [Source: HGNC Symbol; Acc: 2550] |
| 213125_at | 1 | q23.3 | olfactomedin-like 2B [Source: HGNC Symbol; Acc: 24558] |
| 221257_x_at | 5 | q32 | F-box protein 38 [Source: HGNC Symbol; Acc: 28844] |
| 236782_at | 6 | q23.1 | sterile alpha motif domain containing 3 [Source: HGNC |
| | | | Symbol; Acc: 21574] |
| 1554690_a_at | 8 | p11.22 | transforming, acidic coiled-coil containing protein 1 [Source: HGNC |
| | | | Symbol; Acc: 11522] |
| 203431_s_at | 11 | q24.3 | Rho GTPase activating protein 32 [Source: HGNC |
| | | | Symbol; Acc: 17399] |
| 226711_at | 2 | p16.3 | forkhead box N2 [Source: HGNC Symbol; Acc: 5281] |
| 203562_at | 11 | q24.2 | fasciculation and elongation protein zeta 1 (zygin I) [Source: HGNC |
| | | | Symbol; Acc: 3659] |
| 201508_at | 17 | q21.2 | insulin-like growth factor binding protein 4 [Source: HGNC |
| | | | Symbol; Acc: 5473] |
| 209933_s_at | 17 | q25.1 | CD300a molecule [Source: HGNC Symbol; Acc: 19319] |
| 226751_at | 2 | p14 | cannabinoid receptor interacting protein 1 [Source: HGNC |
| | | | Symbol; Acc: 24546] |
| 203303_at | X | p11.4 | dynein, light chain, Tctex-type 3 [Source: HGNC Symbol; Acc: 11694] |
| 1556698_a_at | 4 | q22.1 | GPRIN family member 3 [Source: HGNC Symbol; Acc: 27733] |
| 223454_at | 17 | p13.2 | chemokine (C-X-C motif) ligand 16 [Source: HGNC |
| | | | Symbol; Acc: 16642] |
| 202032_s_at | 15 | q26.1 | mannosidase, alpha, class 2A, member 2 [Source: HGNC |
| | | | Symbol; Acc: 6825] |
| 205779_at | 17 | q21.31 | receptor (G protein-coupled) activity modifying protein 2 |
| | | | [Source: HGNC Symbol; Acc: 9844] |
| 209110_s_at | 6 | p21.32 | ral guanine nucleotide dissociation stimulator-like 2 [Source: HGNC |
| | | | Symbol; Acc: 9769] |
| 209935_at | 3 | q22.1 | ATPase, Ca [symbol] transporting, type 2C, member 1 [Source: HGNC |
| | | | Symbol; Acc: 13211] |
| 214453_s_at | 1 | p31.1 | interferon-induced protein 44 [Source: HGNC Symbol; Acc: 16938] |
| 201063_at | 11 | p13 | reticulocalbin 1, EF-band calcium binding domain [Source: HGNC |
| | | | Symbol; Acc: 9934] |
| 213222_at | 20 | p12.3 | phospholipase C, beta 1 (phosphoinositide-specific) [Source: HGNC |
| | | | Symbol; Acc: 15917] |
| 201069_at | 16 | q12.2 | matrix metallopeptidase 2 (gelatinase A, 72 kDa gelatinase, 72 kDa |
| | | | type IV collagenase) [Source: HGNC Symbol; Acc: 7166] |
| 200661_at | 20 | q13.12 | cathepsin A [Source: HGNC Symbol; Acc: 9251] |
| 213258_at | 2 | q32.1 | tissue factor pathway inhibitor (lipoprotein-associated coagulation |
| | | | inhibitor) [Source: HGNC Symbol; Acc: 11760] |
| 225414_at | 2 | q11.2 | ring finger protein 149 [Source: HGNC Symbol; Acc: 23137] |
| 219397_at | 2 | q33.1 | coenzyme Q10 homolog B (S. cerevisiae) [Source: HGNC |
| | | | Symbol; Acc: 25819] |
| 222127_s_at | 4 | q12 | exocyst complex component 1 [Source: HGNC Symbol; Acc: 30380] |
| 223220_s_at | 3 | q21.1 | poly (ADP-ribose) polymerase family, member 9 [Source: HGNC |
| | | | Symbol; Acc: 24118] |
| 213422_s_at | 1 | p36.33 | matrix-remodelling associated 8 [Source: HGNC Symbol; Acc: 7542] |
| 203153_at | 10 | q23.31 | interferon-induced protein with tetratricopeptide repeats 1 |
| | | | [Source: HGNC Symbol; Acc: 5407] |
| 202100_at | 2 | q14.2 | v-ral simian leukemia viral oncogene homolog B [Source: HGNC |
| | | | Symbol; Acc: 9840] |
| 224895_at | 11 | q22.1 | Yes-associated protein 1 [Source: HGNC Symbol; Acc: 16262] |
| 201368_at | 2 | p21 | ZFP36 ring finger protein-like 2 [Source: HGNC Symbol; Acc: 1108] |
| 212027_at | 14 | q24.2 | RNA binding motif protein 25 [Source: HGNC Symbol; Acc: 23244] |
| 221942_s_at | 4 | q32.1 | guanylate cyclase 1, soluble, alpha 3 [Source: HGNC |
| | | | Symbol; Acc: 4685] |
| 213800_at | 1 | q31.3 | complement factor H [Source: HGNC Symbol; Acc: 4883] |
| 1554999_at | 4 | q21.22 | RasGEF domain family, member 1B [Source: HGNC |
| | | | Symbol; Acc: 24881] |
| 218606_at | 16 | q24.1 | zinc finger, DHHC-type containing 7 [Source: HGNC |
| | | | Symbol; Acc: 18459] |
| 204083_s_at | 9 | p13.3 | tropomyosin 2 (beta) [Source: HGNC Symbol; Acc: 12011] |
| 1553955_at | 2 | p16.3 | protein phosphatase 1, regulatory subunit 21 [Source: HGNC |
| | | | Symbol; Acc: 30595] |
| 228083_at | 12 | p13.33 | calcium channel, voltage-dependent, alpha 2/delta subunit 4 |
| | | | [Source: HGNC Symbol; Acc: 20202] |
| 227304_at | 17 | p11.2 | Smith-Magenis syndrome chromosome region, candidate 8 |
| | | | [Source: HGNC Symbol; Acc: 17921] |
| 225710_at | 3 | q26.33 | guanine nucleotide binding protein (G protein), beta polypeptide 4 |
| | | | [Source: HGNC Symbol; Acc: 20731] |
| 204154_at | 5 | q22.3 | cysteine dioxygenase type 1 [Source: HGNC Symbol; Acc: 1795] |
| 228318_s_at | 4 | p16.3 | cysteine-rich PAK1 inhibitor [Source: HGNC Symbol; Acc: 26619] |
| 227013_at | 13 | q12.11 | large tumor suppressor kinase 2 [Source: HGNC Symbol; Acc: 6515] |
| 1558692_at | 1 | q22 | chrmosome 1 open reading frame 85 [Source: HGNC |
| | | | Symbol; Acc: 29436] |
| 213238_at | 4 | p12 | ATPase, class V, type 10D [Source: HGNC Symbol; Acc: 13549] |
| 201850_at | 2 | p11.2 | capping protein (actin filament), gelsolin-like [Source: HGNC |
| | | | Symbol; Acc: 1474] |
| 209717_at | 1 | p22.1 | ecotropic viral integration site 5 [Source: HGNC Symbol; Acc: 3501] |
| 227388_at | 9 | p21.2 | tumor suppressor candidate 1 [Source: HGNC Symbol; Acc: 31010] |
| 200771_at | 1 | q25.3 | laminin, gamma 1 (formerly LAMB2) [Source: HGNC |
| | | | Symbol; Acc: 6492] |
| 229055_at | 14 | q32.11 | G protein-coupled receptor 68 [Source: HGNC Symbol; Acc: 4519] |
| 200927_s_at | 9 | q33.2 | RAB14, member RAS oncogene family [Source: HGNC |
| | | | Symbol; Acc: 16524] |
| 225447_at | 2 | q24.1 | glycerol-3-phosphate dehydrogenase 2 (mitochondrial) |
| | | | [Source: HGNC Symbol; Acc: 4456] |
| 225525_at | 22 | q11.23 | KIAA1671 [Source: HGNC Symbol; Acc: 29345] |
| 209649_at | 2 | q23.3 | signal transducing adaptor molecule (SH3 domain and ITAM motif) |
| | | | 2 [Source: HGNC Symbol; Acc: 11358] |
| 219492_at | 4 | q12 | cysteine-rich hydrophobic domain 2 [Source: HGNC |
| | | | Symbol; Acc: 1935] |
| 1560060_s_at | 11 | q12.2 | vacuolar protein sorting 37 homolog C (S. cerevisiae) [Source: HGNC |
| | | | Symbol; Acc: 26097] |
| 217947_at | 3 | p22.3 | CKLF-like MARVEL transmembrane domain containing 6 |
| | | | [Source: HGNC Symbol; Acc: 19177] |
| 219078_at | 1 | q41 | G patch domain containing 2 [Source: HGNC Symbol; Acc: 25499] |
| 203854_at | 4 | q25 | complement factor I [Source: HGNC Symbol; Acc: 5394] |
| 209637_s_at | 4 | p16.3 | regulator of G-protein signaling 12 [Source: HGNC |
| | | | Symbol; Acc: 9994] |
| 202252_at | 1 | q21.3 | RAB13, member RAS oncogene family [Source: HGNC |
| | | | Symbol; Acc: 9762] |
| 227373_at | 16 | q22.2 | ataxin 1-like [Source: HGNC Symbol; Acc: 33279] |
| 207469_s_at | X | p22.2 | pirin (iron-binding nuclear protein) [Source: HGNC |
| | | | Symbol; Acc: 30048] |
| 212543_at | 6 | q21 | absent in melanoma 1 [Source: HGNC Symbol; Acc: 356] |
| 208983_s_at | NA | NA | NA |
| 226438_at | 8 | q24.12 | syntrophin, beta 1 (dystrophin-associated protein A1, 59 kDa, basic |
| | | | component 1) [Source: HGNC Symbol; Acc: 11168] |
| 201567_s_at | 3 | p22.2 | golgin A4 [Source: HGNC Symbol; Acc: 4427] |
| 204520_x_at | 22 | q13.33 | bromodomain containing 1 [Source: HGNC Symbol; Acc: 1102] |
| 217828_at | 15 | q22.1 | SAFB-like, transcription modulator [Source: HGNC |
| | | | Symbol; Acc: 20709] |
| 224689_at | 20 | q11.23 | mannosidase, beta A, lysosomal-like [Source: HGNC |
| | | | Symbol; Acc: 15799] |
| 219157_at | 4 | q32.3 | kelch-like family member 2 [Source: HGNC Symbol; Acc: 6353] |
| 232024_at | 7 | q36.1 | GTPase, IMAP family member 2 [Source: HGNC Symbol; Acc: 21789] |
| 219087_at | 9 | q22.31 | asporin [Source: HGNC Symbol; Acc: 14872] |
| 213010_at | 11 | p15.4 | protein kinase C, delta binding protein [Source: HGNC |
| | | | Symbol; Acc: 9400] |
| 219570_at | 20 | p12.1 | kinesin family member 16B [Source: HGNC Symbol; Acc: 15869] |
| 214066_x_at | 9 | p13.3 | natriuretic peptide receptor B/guanylate cyclase B (atrionatriuretic |
| | | | peptide receptor B) [Source: HGNC Symbol; Acc: 7944] |
| 228372_at | 10 | q11.23 | chromosome 10 open reading frame 128 [Source: HGNC |
| | | | Symbol; Acc: 27274] |
| 230276_at | 2 | p24.2 | family with sequence similarity 49, member A [Source: HGNC |
| | | | Symbol; Acc: 25373] |
| 203002_at | 3 | q22.2 | angiomotin like 2 [Source: HGNC Symbol; Acc: 17812] |
| 225688_s_at | 3 | q13.2 | pleckstrin homology-like domain, family B, member 2 |
| | | | [Source: HGNC Symbol; Acc: 29573] |
| 1553678_a_at | 10 | p11.22 | integrin, beta 1 (fibronectin receptor, beta polypeptide, antigen |
| | | | CD29 includes MDF2, MSK12) [Source: HGNC Symbol; Acc: 6153] |
| 203476_at | 6 | q14.1 | trophoblast glycoprotein [Source: HGNC Symbol; Acc: 12004] |
| 201180_s_at | 1 | p13.3 | guanine nucleotide binding protein (G protein), alpha inhibiting |
| | | | activity polypeptide 3 [Source: HGNC Symbol; Acc: 4387] |
| 220141_at | 11 | q24.1 | chromosome 11 open reading frame 63 [Source: HGNC |
| | | | Symbol; Acc: 26288] |
| 227923_at | 22 | q13.33 | SH3 and multiple ankyrin repeat domains 3 [Source: HGNC |
| | | | Symbol; Acc: 14294] |
| 235334_at | 1 | p31.1 | ST6 (alpha-N-acetyl-neuraminyl-2,3-beta-galactosyl-1,3)-N- |
| | | | acetylgalactosaminide alpha-2,6-sialyltransferase 3 [Source: HGNC |
| | | | Symbol; Acc: 19343] |
| 235411_at | 6 | p22.1 | piggyBac transposable element derived 1 [Source: HGNC |
| | | | Symbol; Acc: 19398] |
| 216689_x_at | 11 | p11.2 | Rho GTPase activating protein 1 [Source: HGNC Symbol; Acc: 673] |
| 202932_at | 18 | p11.32 | v-yes-1 Yamaguchi sarcoma viral oncogene homolog 1 |
| | | | [Source: HGNC Symbol; Acc: 12841] |
| 212293_at | 1 | p13.2 | homeodomain interacting protein kinase 1 [Source: HGNC |
| | | | Symbol; Acc: 19006] |
| 225618_at | 17 | q21.31 | Rho GTPase activating protein 27 [Source: HGNC |
| | | | Symbol; Acc: 31813] |
| 201242_s_at | 1 | q24.2 | ATPase, Nasun transporting, beta 1 polypeptide [Source: HGNC |
| | | | Symbol; Acc: 804] |
| 203583_at | 2 | q11.2 | unc-50 homolog (C. elegans) [Source: HGNC Symbol; Acc: 16046] |
| 1557938_s_at | 17 | q21.2 | polymerase I and transcript release factor [Source: HGNC |
| | | | Symbol; Acc: 9688] |
| 242321_at | 1 | q41 | protein tyrosine phosphatase, non-receptor type 14 [Source: HGNC |
| | | | Symbol; Acc: 9647] |
| 212950_at | 6 | p12.3 | G protein-coupled receptor 116 [Source: HGNC Symbol; Acc: 19030] |
| 205638_at | 7 | p14.2 | acyloxyacyl hydrolase (neutrophil) [Source: HGNC Symbol; Acc: 548] |
| 223209_s_at | 15 | q26.3 | VCP-interacting membrane protein [Source: HGNC |
| | | | Symbol; Acc: 30396] |
| 208131_s_at | 20 | q13.13 | prostaglandin I2 (prostacyclin) synthase [Source: HGNC |
| | | | Symbol; Acc: 9603] |
| 227833_s_at | 12 | q13.3 | methyl-CpG binding domain protein 6 [Source: HGNC |
| | | | Symbol; Acc: 20445] |
| 208991_at | 17 | q21.2 | signal transducer and activator of transcription 3 (acute-phase |
| | | | response factor) [Source: HGNC Symbol; Acc: 11364] |
| 216361_s_at | 8 | p11.21 | K(lysine) acetyltransferase 6A [Source: HGNC Symbol; Acc: 13013] |
| 1554106_at | 2 | q33.2 | neurobeachin-like 1 [Source: HGNC Symbol; Acc: 20681] |
| 209357_at | 6 | q24.1 | Cbp/p300-interacting transactivator, with Glu/Asp-rich carboxy- |
| | | | terminal domain, 2 [Source: HGNC Symbol; Acc: 1987] |
| 53720_at | 19 | p13.2 | chromosome 19 open reading frame 66 [Source: HGNC |
| | | | Symbol; Acc: 25649] |
| 228964_at | 6 | q21 | PR domain containing 1, with ZNF domain [Source: HGNC |
| | | | Symbol; Acc: 9346] |
| 228624_at | 4 | q32.1 | transmembrane protein 144 [Source: HGNC Symbol; Acc: 25633] |
| 225626_at | 8 | q21.13 | phosphoprotein associated with glycosphingolipid microdomains 1 |
| | | | [Source: HGNC Symbol; Acc: 30043] |
| 225885_at | 12 | q22 | early endosome antigen 1 [Source: HGNC Symbol; Acc: 3185] |
| 236172_at | 14 | q12 | leukotriene B4 receptor [Source: HGNC Symbol; Acc: 6713] |
| 201953_at | 15 | q26.1 | calcium and integrin binding 1 (calmyrin) [Source: HGNC |
| | | | Symbol; Acc: 16920] |
| 213069_at | 3 | q21.2 | heart development protein with EGF-like domains 1 [Source: HGNC |
| | | | Symbol; Acc: 29227] |
| 226885_at | 6 | q22.31 | ring fnger protein 217 [Source: HGNC Symbol; Acc: 21487] |
| 203038_at | 6 | q22.33 | protein tyrosine phosphatase, receptor type, K [Source: HGNC |
| | | | Symbol; Acc: 9674] |
| 240703_s_at | 15 | q22.31 | HECT and RLD domain containing E3 ubiquitin protein ligase family |
| | | | membrane 1 [Source: HGNC Symbol; Acc: 4867] |
| 221641_s_at | X | p22.11 | acyl-CoA thioesterase 9 [Source: HGNC Symbol; Acc: 17152] |
| 210845_s_at | 19 | q13.31 | plasminogen activator, urokinase receptor [Source: HGNC |
| | | | Symbol; Acc: 9053] |
| 212097_at | 7 | q31.2 | caveolin 1, caveolae protein, 22 kDa [Source: HGNC |
| | | | Symbol; Acc: 1527] |
| 243198_at | 15 | q21.3 | testis expressed 9 [Source: HGNC Symbol; Acc: 29585] |
| 204122_at | 19 | q13.12 | TYRO protein tyrosine kinase binding protein [Source: HGNC |
| | | | Symbol; Acc: 12449] |
| 226343_at | 15 | q22.31 | dipeptidyl-peptidase 8 [Source: HGNC Symbol; Acc: 16490] |
| 205715_at | 4 | p15.32 | bone narrow stromal cell antigen 1 [Source: HGNC |
| | | | Symbol; Acc: 1118] |
| 211980_at | 13 | q34 | collagen, type IV, alpha 1 [Source: HGNC Symbol; Acc: 2202] |
| 213572_s_at | 6 | p25.2 | serpin peptidase inhibitor, clade B (ovalbumin), member 1 |
| | | | [Source: HGNC Symbol; Acc: 3311] |
| 209047_at | 7 | p14.3 | aquaporin 1 (Colton blood group) [Source: HGNC Symbol; Acc: 633] |
| 214077_x_at | 17 | p12 | Meis homeobox 3 pseudogene 1 [Source: HGNC Symbol; Acc: 7002] |
| 202946_s_at | 20 | p12.2 | BTB (POZ) domain containing 3 [Source: HGNC Symbol; Acc: 15854] |
| 242953_at | 19 | q13.31 | zinc finger protein 234 [Source: HGNC Symbol; Acc: 13027] |
| 218380_at | NA | NA | NA |
| 215000_s_at | 2 | p22.2 | fasciculation and elongation protein zeta 2 (zygin II) [Source: HGNC |
| | | | Symbol; Acc: 3660] |
| 229238_at | 17 | p13.3 | chromosome 17 open reading frame 97 [Source: HGNC |
| | | | Symbol; Acc: 33800] |
| 219700_at | 17 | q12 | plexin domain containing 1 [Source: HGNC Symbol; Acc: 20945] |
| 202432_at | 10 | q22.2 | protein phosphatase 3, catalytic subunit, beta isozyme |
| | | | [Source: HGNC Symbol; Acc: 9315] |
| 1558711_at | 4 | q22.1 | FAM13A antisense RNA 1 [Source: HGNC Symbol; Acc: 19370] |
| 202304_at | 13 | q14.2 | fibronectin type III domain containing 3A [Source: HGNC |
| | | | Symbol; Acc: 20296] |
| 213429_at | 10 | q21.1 | bicaudal C homolog 1 (Drosophila) [Source: HGNC |
| | | | Symbol; Acc: 19351] |
| 203388_at | 17 | p13.2 | arrestin, beta 2 [Source: HGNC Symbol; Acc: 712] |
| 205140_at | 1 | p31.1 | fucose-1-phosphate guanylyltransferase [Source: HGNC |
| | | | Symbol; Acc: 3825] |
| 226594_at | 14 | q24.3 | ectonucleoside triphosphate diphospphohydrolase 5 [Source: HGNC |
| | | | Symbol; Acc: 3367] |
| 219506_at | 1 | q21.2 | chromosome 1 open reading frame 54 [Source: HGNC |
| | | | Symbol; Acc: 26258] |
| 209230_s_at | 16 | p11.2 | nuclear protein, transcriptional regulator, 1 [Source: HGNC |
| | | | Symbol; Acc: 29990] |
| 205498_at | 5 | p13.1 | growth hormone receptor [Source: HGNC Symbol; Acc: 4263] |

TABLE 4

| | | | | Chromo- | | |
|---|---|---|---|---|---|---|
| Gene | cox.pvalues | cox.coefficients | ensembl_gene _id | some | band | description |
| CAMTA2 | 0.00000203 | −4.668235881 | ENSG00000108509 | 17 | p13.2 | calmodulin binding transcription activator 2 [Source: HGNC Symbol;Acc: 18807] |
| ODF3B | 0.00003813 | −2.506221601 | ENSG00000177989 | 22 | q13.33 | outer dense fiber of sperm tails 3B [Source: HGNC Symbol;Acc: 34388] |
| SCO2 | 0.00005664 | −2.545936327 | ENSG00000130489 | 22 | q13.33 | SCO2 cytochrome c oxidase assembly protein [Source: HGNC Symbol;Acc: 10604] |
| PSTPIP1 | 0.00005675 | −3.099610719 | ENSG00000140368 | 15 | q24.3 | proline-serine-threonine phosphatase interacting protein 1 [Source: HGNC Symbol;Acc: 9580] |
| ZNF25 | 0.00006246 | −2.650047611 | ENSG00000175395 | 10 | p11.1 | zinc finger protein 25 [Source: HGNC Symbol;Acc: 13043] |
| PHLDA1 | 0.00008490 | −1.39157215 | ENSG00000139289 | 12 | q21.2 | pleckstrin homology-like domain, family A, member 1 [Source: HGNC Symbol;Acc: 8933] |
| LOC100509205 | 0.00008899 | 3.284490596 | NA | NA | NA | NA |
| NINL | 0.00009498 | −3.750225037 | ENSG00000101004 | 20 | p11.21 | ninein-like [Source: HGNC Symbol;Acc: 29163] |
| IL11RA | 0.00011009 | −2.428243399 | ENSG00000137070 | 9 | p13.3 | interleukin 11 receptor, alpha [Source: HGNC Symbol;Acc: 5967] |
| IL18RAP | 0.00013238 | −2.002156881 | ENSG00000115607 | 2 | q12.1 | interleukin 18 receptor accessory protein [Source: HGNC Symbol;Acc: 5989] |
| CC2D1B | 0.00013484 | −3.165981531 | ENSG00000154222 | 1 | p32.3 | coiled-coil and C2 domain containing 1B [Source: HGNC Symbol;Acc: 29386] |
| FAM219A | 0.00016883 | −2.594263538 | ENSG00000164970 | 9 | p13.3 | family with sequence similarity 219, member A [Source: HGNC Symbol;Acc: 19920] |
| IL18R1 | 0.00019551 | −1.244126139 | ENSG00000115604 | 2 | q12.1 | interleukin 18 receptor 1 [Source: HGNC Symbol;Acc: 5988] |
| CASP7 | 0.00019755 | −1.567912334 | ENSG00000165806 | 10 | q25.3 | caspase 7, apoptosis-related cysteine peptidase [Source: HGNC Symbol;Acc: 1508] |
| CD5 | 0.00022052 | −2.463614971 | ENSG00000110448 | 11 | q12.2 | CD5 molecule [Source: HGNC Symbol;Acc: 1685] |
| COX10 | 0.00023739 | −2.388350273 | ENSG00000006695 | 17 | p12 | cytochrome c oxidase assembly homolog 10 (yeast) [Source: HGNC Symbol;Acc: 2260] |
| LYST | 0.00033014 | −0.979162015 | ENSG00000143669 | 1 | q42.3 | lysosomal trafficking regulator [Source: HGNC Symbol;Acc: 1968] |
| LOC100507507 | 0.00036569 | −2.223497444 | NA | NA | NA | NA |
| TRIP6 | 0.00037027 | −2.212398882 | ENSG00000087077 | 7 | q22.1 | thyroid hormone receptor interactor 6 [Source: HGNC Symbol;Acc: 12311] |
| WDR27 | 0.00037479 | 2.047182081 | ENSG00000184465 | 6 | q27 | WD repeat domain 27 [Source: HGNC Symbol;Acc: 21248] |
| SLFN12 | 0.00037578 | −1.198035184 | ENSG00000172123 | 17 | q12 | schlafen family member 12 [Source: HGNC Symbol;Acc: 25500] |
| TBC1D4 | 0.00038960 | −0.865903169 | ENSG00000136111 | 13 | q22.2 | TBC1 domain family, member 4 [Source: HGNC Symbol;Acc: 19165] |
| ACADVL | 0.00040722 | −1.831475638 | ENSG00000072778 | 17 | p13.1 | acyl-CoA dehydrogenase, very long chain [Source: HGNC Symbol;Acc: 92] |
| PHEX | 0.00042337 | −1.148081776 | ENSG00000102174 | X | p22.11 | phosphate regulating endopeptidase homolog, X-linked [Source: HGNC Symbol;Acc: 8918] |
| TOR1B | 0.00048257 | −2.788588939 | ENSG00000136816 | 9 | q34.11 | torsin family 1, member B (torsin B) [Source: HGNC Symbol;Acc: 11995] |
| KIF26B | 0.00050893 | −0.820664008 | ENSG00000162849 | 1 | q44 | kinesin family member 26B [Source: HGNC Symbol;Acc: 25484] |
| COQ2 | 0.00052599 | −2.524800127 | ENSG00000173085 | 4 | q21.23 | coenzyme Q2 4-hydroxybenzoate polyprenyltransferase [Source: HGNC Symbol;Acc: 25223] |
| NDUFV3 | 0.00054696 | −2.206346662 | ENSG00000160194 | 21 | q22.3 | NADH dehydrogenase (ubiquinone) flavoprotein 3, 10 kDa [Source: HGNC Symbol;Acc: 7719] |
| CNTLN | 0.00056050 | −1.199738102 | ENSG00000044459 | 9 | p22.2 | centlein, centrosornal protein [Source: HGNC Symbol;Acc: 23432] |
| SLC6A6 | 0.00056079 | −1.66525648 | ENSG00000131389 | 3 | p25.1 | solute carrier family 6 (neurotransmitter transporter, taurine), member 6 [Source: HGNC Symbol;Acc: 11052] |
| TUBG2 | 0.00057975 | −2.766415519 | ENSG00000037042 | 17 | q21.2 | tubulin, gamma 2 [Source: HGNC Symbol;Acc: 12419] |
| LPAR6 | 0.00059706 | −0.810738084 | ENSG00000139679 | 13 | q14.2 | lysophosphatidic acid receptor 6 [Source: HGNC Symbol;Acc: 15520] |
| SLC12A7 | 0.00060983 | −1.723437657 | ENSG00000113504 | 5 | p15.33 | solute carrier family 12 (potassium/chloride transporters), member 7 [Source: HGNC Symbol;Acc: 10915] |
| TYMP | 0.00061216 | −1.832645096 | ENSG00000025708 | 22 | q13.33 | thymidine phosphorylase [Source: HGNC Symbol;Acc: 3148] |
| TMOD2 | 0.00062660 | −1.291604457 | ENSG00000128872 | 15 | q21.2 | tropomodulin 2 (neuronal) [Source: HGNC Symbol;Acc: 11872] |
| SUN2 | 0.00063540 | −5.074770677 | ENSG00000100242 | 22 | q13.1 | Sad1 and UNC84 domain containing 2 [Source: HGNC Symbol;Acc: 14210] |
| NRBP2 | 0.00066284 | −1.371485291 | ENSG00000185189 | 8 | q24.3 | nuclear receptor binding protein 2 [Source: HGNC Symbol;Acc: 19339] |
| ZSWIM7 | 0.00067007 | −1.458321834 | ENSG00000214941 | 17 | p12 | zinc finger, SWIM-type containing 7 [Source: HGNC Symbol;Acc: 26993] |
| S100B | 0.00067297 | −1.792059143 | ENSG00000160307 | 21 | q22.3 | S100 calcium binding protein B [Source: HGNC Symbol;Acc: 10500] |
| ITGB2-AS1 | 0.00067370 | −0.863312228 | ENSG00000227039 | 21 | q22.3 | ITGB2 antisense RNA 1 [Source: HGNC Symbol;Acc: 44304] |
| LOC730227 | 0.00067711 | 3.052297063 | NA | NA | NA | NA |
| ALLC | 0.00070484 | 1.173487594 | ENSG00000151360 | 2 | p25.3 | allantoicase [Source: HGNC Symbol;Acc: 17377] |
| ECHDC2 | 0.00074255 | −1.182987095 | ENSG00000121310 | 1 | p32.3 | enoyl CoA hydratase domain containing 2 [Source: HGNC Symbol;Acc: 23408] |
| VPS26B | 0.00076061 | −1.959803305 | ENSG00000151502 | 11 | q25 | vacuolar protein sorting 26 homolog B (S. pombe) [Source: HGNC Symbol;Acc: 28119] |
| MGC16142 | 0.00081730 | 3.447869406 | NA | NA | NA | NA |
| ADAP2 | 0.00085355 | −1.423623909 | ENSG00000184060 | 17 | q11.2 | ArfGAP with dual PH domains 2 [Source: HGNC Symbol;Acc: 16487] |
| TCL6 | 0.00085843 | 1.001037623 | ENSG00000187621 | 14 | q32.13 | T-cell leukemia/lymphoma 6 (non-protein coding) [Source: HGNC Symbol;Acc: 13463] |
| IFIT5 | 0.00086743 | −1.127640551 | ENSG00000152778 | 10 | q23.31 | interferon-induced protein with tetratricopeptide repeats 5 [Source: HGNC Symbol;Acc: 13328] |
| LDOC1L | 0.00083004 | −1.303614625 | ENSG00000188636 | 22 | q13.31 | leucine zipper, down-regulated in cancer 1-like [Source: HGNC Symbol;Acc: 13343] |
| NUDT16 | 0.00091516 | −1.407527098 | ENSG00000198585 | 3 | q22.1 | nudix (nucleoside diphosphate linked moiety X)-type motif 16 [Source: HGNC Symbol;Acc: 26442] |
| FLII | 0.00093525 | −1.671920539 | ENSG00000177731 | 17 | p11.2 | flightless I homolog (Drosophila) [Source: HGNC Symbol;Acc: 3750] |
| HEXIM1 | 0.00096902 | −1.893455283 | ENSG00000186834 | 17 | q21.31 | hexamethylene bis-acetamide inducible 1 [Source: HGNC Symbol;Acc: 24953] |
| SGSM2 | 0.00099078 | −1.460523274 | ENSG00000141258 | 17 | p13.3 | small G protein signaling modulator 2 [Source: HGNC Symbol;Acc: 29026] |
| FXYD2 | 0.00100378 | −1.494412258 | ENSG00000137731 | 11 | q23.3 | FXYD domain containing ion transport regulator 2 [Source: HGNC Symbol;Acc: 4026] |
| RDH10 | 0.00100828 | −1.263627433 | ENSG00000121039 | 8 | q21.11 | retinol dehydrogenase 10 (all-trans) [Source: HGNC Symbol;Acc: 19975] |
| RHOBTB2 | 0.00100852 | 1.443814978 | ENSG00000008853 | 8 | p21.3 | Rho-related BTB domain containing 2 [Source: HGNC Symbol;Acc: 18756] |
| PDE8A | 0.00103756 | −1.366305067 | ENSG00000073417 | 15 | q25.3 | phosphodiesterase 8A [Source: HGNC Symbol;Acc: 8793] |
| KIAA1598 | 0.00106273 | −0.883804986 | ENSG00000187164 | 10 | q25.3 | KIAA1598 [Source: HGNC Symbol;Acc: 29319] |
| ACSL1 | 0.00106684 | −1.1398898 | ENSG00000151726 | 4 | q35.1 | acyl-CoA synthetase long-chain family member 1 [Source: HGNC Symbol;Acc: 3569] |
| CAPN2 | 0.00111892 | −0.673819472 | ENSG00000162909 | 1 | q41 | calpain 2, (m/ll) large subunit [Source: HGNC Symbol;Acc: 1479] |
| EPM2A | 0.00112005 | 2.042733065 | ENSG00000112425 | 6 | q24.3 | epilepsy, progressive myoclonus type 2A, Lafora disease (laforin) [Source: HGNC Symbol;Acc: 3413] |
| SPG20 | 0.00112318 | −1.345465148 | ENSG00000133104 | 13 | q13.3 | spastic paraplegia 20 (Troyer syndrome) [Source: HGNC Symbol;Acc: 18514] |
| NUDT9P1 | 0.00113163 | 1.948144791 | NA | NA | NA | NA |
| FHOD1 | 0.00116819 | −2.500243302 | ENSG00000135723 | 16 | q22.1 | formin homology 2 domain containing 1 [Source: HGNC Symbol;Acc: 17905] |
| OSCAR | 0.00118671 | −2.271018361 | ENSG00000170909 | 19 | q13.42 | osteoclast associated, immunoglobulin-like receptor [Source: HGNC Symbol;Acc: 29960] |
| ARMCX1 | 0.00119573 | −1.079753587 | ENSG00000126947 | X | q22.1 | armadillo repeat containing, X-linked 1 [Source: HGNC Symbol,Acc: 18073] |
| SELP | 0.00122190 | −0.903200375 | ENSG00000174175 | 1 | q24.2 | selectin P (granule membrane protein 140 kDa, antigen CD62) [Source: HGNC Symbol;Acc: 10721] |
| CD200R1 | 0.00126705 | −0.920571979 | ENSG00000163606 | 3 | q13.2 | CD200 receptor 1 [Source: HGNC Symbol;Acc: 24235] |
| PLEKHA7 | 0.00128422 | −1.287862112 | ENSG00000166689 | 11 | p15.1 | pleckstrin homology domain containing, family A member 7 [Source: HGNC Symbol;Acc: 27049] |
| GNAQ | 0.00133109 | −0.799757937 | ENSG00000156052 | 9 | q21.2 | guanine nucleotide binding protein (G protein), q polypeptide [Source: HGNC Symbol;Acc: 4390] |
| ZBTB4 | 0.00135017 | −1.220347435 | ENSG00000174282 | 17 | p13.1 | zinc finger and BTB domain containing 4 [Source: HGNC Symbol;Acc: 23847] |
| CLEC12A | 0.00136635 | −1.624654383 | ENSG00000172322 | 12 | p13.2 | C-type lectin domain family 12, member A [Source: HGNC Symbol;Acc: 31713] |
| PTSD | 0.00139038 | −3.41677807 | ENSG00000241878 | 22 | p12.2 | phosphatidylserine decarboxylase [Source: HGNC Symbol;Acc: 8999] |
| ALDH2 | 0.00141785 | −0.672902178 | ENSG00000111275 | 12 | q24.12 | aldehyde dehydrogenase 2 family (mitochondrial) [Source: HGNC Symbol;Acc: 404] |
| IL6R | 0.00142894 | −0.755163035 | ENSG00000160712 | 1 | p21.3 | interleukin 6 receptor [Source: HGNC Symbol;Acc: 6019] |
| MRC2 | 0.00144402 | −1.526866671 | ENSG00000011028 | 17 | q23.2 | mannose receptor, C type 2 [Source: HGNC Symbol;Acc: 16875] |
| TSEN34 | 0.00145956 | −2.689190004 | ENSG00000170892 | 19 | q13.42 | tRNA splicing endonuclease 34 homolog (S. cerevisiae) [Source: HGNC Symbol;Acc: 15506] |
| GALM | 0.00147988 | −0.995059373 | ENSG00000143891 | 2 | p22.1 | galactose mutarotase (aldose 1-epimerase) [Source: HGNC Symbol;Acc: 24063] |
| EMP3 | 0.00148006 | −1.056401849 | ENSG00000142227 | 19 | q13.33 | epithelial membrane protein 3 [Source: HGNC Symbol;Acc: 3335] |
| SAT2 | 0.00157137 | −2.792215867 | ENSG00000141504 | 17 | p13.1 | spermidine/sperrnine N1-acetyltransferase family member 2 [Source: HGNC Symbol;Acc: 23160] |
| TTC31 | 0.00157244 | −1.63766409 | ENSG00000115282 | 2 | p13.1 | tetratricopeptide repeat domain 31 [Source: HGNC Symbol;Acc: 25759] |
| CES2 | 0.00159167 | −2.024818272 | ENSG00000172831 | 16 | q22.1 | carboxylesterase 2 [Source: HGNC Symbol;Acc: 1864] |
| AGPAT9 | 0.00159441 | −1.35239458 | ENSG00000138678 | 4 | q21.23 | 1-acylglycerol 3 phosphate O-acyltransferase 9 [Source: HGNC Symbol;Acc: 28157] |
| RBM43 | 0.00161986 | −1.428737679 | ENSG00000184898 | 2 | q23.3 | RNA binding motif protein 43 [Source: HGNC Symbol;Acc: 24790] |
| CXCL14 | 0.00162663 | −0.467340849 | ENSG00000145824 | 5 | q31.1 | chemokine (C-X-C motif) ligand 14 [Source: HGNC Symbol;Acc: 10640] |
| PLEKHA1 | 0.00167197 | −0.919870988 | ENSG00000107679 | 10 | q26.13 | pleckstrin homology domain containing, family A (phosphoinositide binding specific) member 1 [Source: HGNC |
| | | | | | | Symbol;Acc: 14335] |
| SLC35D2 | 0.00168394 | −1.418796477 | ENSG00000130958 | 9 | q22.32 | solute carrier family 35 (UDP-GlcNAc/UDP-glucose transporter), member D2 [Source: HGNC Symbol;Acc: |
| | | | | | | 20799] |
| LOC654841 | 0.00170773 | 4.303627408 | NA | NA | NA | NA |
| RFPL3S | 0.00171657 | −2.35571089 | ENSG00000205853 | 22 | q12.3 | RFPL3 antisense [Source: HGNC Symbol;Acc: 9981] |
| PLCB2 | 0.00175948 | −2.173217301 | ENSG00000137841 | 15 | q15.1 | phospholipase C, beta 2 [Source: HGNC Symbol;Acc: 9055] |
| DPP4 | 0.00176325 | −1.056413733 | ENSG00000197635 | 2 | q24.2 | dipeptidyl-peptidase 4 [Source: HGNC Symbol;Acc: 3009] |
| FAM219B | 0.00181328 | −1.464593174 | ENSG00000178761 | 15 | q24.1 | family with sequence similarity 219, member B [Source: HGNC Symbol;Acc: 24695] |
| TSPAN2 | 0.00181539 | −1.024722523 | ENSG00000134198 | 1 | p13.2 | tetraspanin 2 [Source: HGNC Symbol;Acc: 20659] |
| SLC2A4RG | 0.00183651 | −2.092522041 | ENSG00000125520 | 20 | q13.33 | SLC2A4 regulator [Source: HGNC Symbol;Acc: 15930] |
| TCF7L2 | 0.00183773 | −1.072368328 | ENSG00000148737 | 10 | q25.2 | transcription factor 7-like 2 (T-cell specific, HMG-box) [Source: HGNC Symbol;Acc: 11641] |
| C10orf76 | 0.00184603 | −1.832924913 | ENSG00000120029 | 10 | q24.32 | chromosome 10 open reading frame 76 [Source: HGNC Symbol;Acc: 25788] |
| NAGA | 0.00187105 | −1.371486854 | ENSG00000198951 | 22 | q13.2 | N-acetylgalactosaminidase, alpha-[Source: HGNC Symbol;Acc: 7631] |
| RIPK3 | 0.00190047 | −3.462098915 | ENSG00000129465 | 14 | q12 | receptor-interacting serine-threonine kinase 3 [Source: HGNC Symbol;Acc: 10021] |
| CD1E | 0.00197070 | −1.121895164 | ENSG00000158488 | 1 | q23.1 | CD1e molecule [Source: HGNC Symbol;Acc: 1638] |
| HAVCR2 | 0.00202258 | −0.729699479 | ENSG00000135077 | 5 | q33.3 | hepatitis A virus cellular receptor 2 [Source: HGNC Symbol;Acc: 18437] |

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

What is claimed is:

1. A method for predicting the clinical sensitivity of a diffuse large B-cell lymphoma (DLBCL) to treatment with lenalidomide comprising:
(a) obtaining a first biological sample from a first patient having a DLBCL;
(b) measuring the level of expression of one, two, three, four, five or more of the genes identified in Table 3;
(c) comparing the level of expression of the one, two, three, four, five or more of the genes identified in Table 3 in the first biological sample with the level of expression of the same genes in a second biological sample from a second patient having a DLBCL, wherein the DLBCL in the second patient is clinically insensitive to lenalidomide, or pharmaceutically acceptable salts, solvates or isomers thereof, and
wherein the differential expression of the one, two, three, four, five or more of the genes in the first biological sample relative to the level of expression of the one, two, three, four, five or more of the genes in the second biological sample indicates that the DLBCL in the first patient will be clinical sensitive to treatment with lenalidomide, or pharmaceutically acceptable salts, solvates or isomers thereof.

2. A method for predicting the clinical sensitivity of a DLBCL to treatment with lenalidomide, or pharmaceutically acceptable salts, solvates or isomers thereof, comprising:
(a) obtaining a first biological sample from a first patient having a DLBCL;
(b) measuring the level of expression of one, two, three, four, five or more of the genes identified in Table 4;
(c) comparing the level of expression of the one, two, three, four, five or more of the genes identified in Table 4 in the first biological sample with the level of expression of the same genes in a second biological sample from a second patient having a DLBCL, wherein the DLBCL in the second patient is clinically insensitive to lenalidomide, or pharmaceutically acceptable salts, solvates or isomers thereof, and
wherein the differential expression of the one, two, three, four, five or more of the genes in the first biological sample relative to the level of expression of the one, two, three, four, five or more of the genes in the second biological sample indicates that the DLBCL in the first patient will be clinical sensitive to treatment with lenalidomide.

3. A method for predicting the clinical sensitivity of a DLBCL to treatment with lenalidomide, or pharmaceutically acceptable salts, solvates or isomers thereof, comprising:
(a) obtaining a first biological sample from a first patient having a DLBCL;
(b) measuring the level of expression of one, two, three, four, five or more of the genes identified in Table 1; and
(c) comparing the level of expression of the one, two, three, four, five or more of the genes identified in Table 1 in the first biological sample with the level of expression of the same genes in a second biological sample from a second patient having a DLBCL, wherein the DLBCL cancer in the second patient is clinically insensitive to lenalidomide, or pharmaceutically acceptable salts, solvates or isomers thereof, and
wherein a higher level of expression of the one, two, three, four, five or more of the genes in the first biological sample relative to the level of expression of the one, two, three, four, five or more of the genes in the second biological sample indicates that the DLBCL in the first patient will be clinical sensitive to treatment with lenalidomide, or pharmaceutically acceptable salts, solvates or isomers thereof.

4. A method for predicting the clinical sensitivity of a DLBCL to treatment with lenalidomide, or pharmaceutically acceptable salts, solvates or isomers thereof, comprising:
(a) obtaining a first biological sample from a first patient having a DLBCL;
(b) measuring the level of expression of one, two, three, four, five or more of the genes identified in Table 2; and
(c) comparing the level of expression of the one, two, three, four, five or more of the genes identified in Table 2 in the first biological sample with the level of expression of the same genes in a second biological sample is from a second patient having a DLBCL, wherein the DLBCL in the second patient is clinically insensitive to lenalidomide, or pharmaceutically acceptable salts, solvates or isomers thereof, and
wherein a lower level of expression of the one, two, three, four, five or more of the genes in the first biological sample relative to the level of expression of the one, two, three, four, five or more of the genes in the second biological sample indicates that the DLBCL in the first patient will be clinical sensitive to treatment with lenalidomide, or pharmaceutically acceptable salts, solvates or isomers thereof.

5. A method for predicting the clinical sensitivity of a DLBCL to treatment with lenalidomide, or pharmaceutically acceptable salts, solvates or isomers thereof, comprising:
(a) obtaining a first tumor sample from a first patient having a DLBCL;
(b) measuring the proportion of dendritic cells in the first tumor sample; and
(c) comparing the proportion of dendritic cells in the first tumor sample with the proportion of dendritic cells in a second tumor sample from a second patient having a DLBCL, wherein the second patient's DLBCL is clinically insensitive to treatment with lenalidomide, or pharmaceutically acceptable salts, solvates or isomers thereof, and
wherein a higher proportion of dendritic cells in the first tumor sample relative the proportion of dendritic cells in the second tumor sample indicates that the DLBCL in the first patient will be clinical sensitive to treatment with lenalidomide, or pharmaceutically acceptable salts, solvates or isomers thereof.

6. A methods for predicting the clinical sensitivity of a DLBCL to treatment with lenalidomide, or pharmaceutically acceptable salts, solvates or isomers thereof, comprising:
(a) obtaining a first tumor sample from a first patient having a DLBCL;
(b) measuring the proportion of plasma cells in the first tumor sample; and
(c) comparing the proportion of plasma cells in the first tumor sample with the proportion of plasma cells in a second tumor sample from a second patient having a DLBCL, wherein the second patient's DLBCL is clinically insensitive to treatment with lenalidomide, or pharmaceutically acceptable salts, solvates or isomers thereof, and
wherein a higher proportion of plasma cells in the first tumor sample relative the proportion of plasma cells in the second tumor sample indicates that the DLBCL in the first patient will be clinical sensitive to treatment with lenalidomide, or pharmaceutically acceptable salts, solvates or isomers thereof.

7. A method for predicting the clinical sensitivity of a DLBCL to treatment with lenalidomide, or pharmaceutically acceptable salts, solvates or isomers thereof, comprising:
(a) obtaining a first biological sample from a first patient having a DLBCL
(b) measuring the expression of the genes or a certain subset of genes set forth in Table 1, 2, 3, or 4 in the first biological sample; and
(c) comparing the gene expression profile of the genes or subset of genes in the first biological sample to (i) the gene expression profile of the genes or subset of genes in tumor samples from patients having DLBCL which are clinically sensitive to lenalidomide, or pharmaceutically acceptable salts, solvates or isomers thereof, and (ii) the gene expression of the genes or subset of genes in tumor samples from patients having DLBCL which are clinically insensitive to lenalidomide, or pharmaceutically acceptable salts, solvates or isomers thereof,
wherein a gene expression profile for the genes or subset of genes in the first biological sample similar to the gene expression profile for the genes or subset of genes in tumor samples from patients having DLBCL which are clinically sensitive to lenalidomide, or pharmaceutically acceptable salts, solvates or isomers thereof, indicates that the DLBCL in the first patient will be clinical sensitive to treatment with lenalidomide, and a gene expression profile for the genes or subset of genes in first biological sample similar to the gene expression profile for the genes or subset of genes in tumor samples from patients having DLBCL which are clinically insensitive to lenalidomide, or pharmaceutically acceptable salts, solvates or isomers thereof, indicates that the DLBCL of the first patient will be clinically insensitive to treatment with lenalidomide, or pharmaceutically acceptable salts, solvates or isomers thereof.

8. A method for predicting the clinical sensitivity of a DLBCL to treatment with lenalidomide, or pharmaceutically acceptable salts, solvates or isomers thereof, comprising:
(a) obtaining a first tumor sample from a first patient having a hematological cancer;
(b) measuring the proportion of immune cells in the first tumor sample; and
(c) comparing the proportion of the immune cells in the first tumor sample to (i) the proportion of the same immune cells in tumor samples from patients having DLBCL which are clinically sensitive to lenalidomide, or pharmaceutically acceptable salts, solvates or isomers thereof, and (ii) the proportion of the same immune cells in tumor samples from patients having DLBCL which are clinically insensitive to lenalidomide, or pharmaceutically acceptable salts, solvates or isomers thereof,
wherein a proportion of the immune cells in the first tumor sample similar to the proportion of the same immune cells in tumor samples from patients having DLBCL which are clinically sensitive to lenalidomide, or pharmaceutically acceptable salts, solvates or isomers thereof, indicates that the DLBCL in the first patient will be clinical sensitive to treatment with lenalidomide, or pharmaceutically acceptable salts, solvates or isomers thereof, and a proportion of the immune cells in the first tumor sample similar to the proportion of the same immune cells in tumor samples from patients having DLBCL which are clinically insensitive to lenalidomide, or pharmaceutically acceptable salts, solvates or isomers thereof, indicates that the DLBCL in the first patient will be clinical insensitive to treatment with lenalidomide, or pharmaceutically acceptable salts, solvates or isomers thereof.

9. A method for managing or treating a DLBCL comprising:
(a) obtaining a first biological sample from a first patient having a DLBCL;
(b) measuring the level of expression of one, two, three, four, five or more of the genes identified in Table 3;
(c) comparing the level of expression of the one, two, three, four, five or more of the genes identified in Table 3 in the first biological sample with the level of expression of the same genes in a second biological sample from a second patient having a DLBCL, wherein the DLBCL in the second patient is clinically insensitive to lenalidomide, or pharmaceutically acceptable salts, solvates or isomers thereof; and
(d) administering lenalidomide, or pharmaceutically acceptable salts, solvates or isomers thereof, to the first patient if the one, two, three, four, five or more of the genes in the first biological sample are differentially expressed relative to the level of expression of the one, two, three, four, five or more of the genes in the second biological sample.

10. A method for managing or treating a DLBCL comprising:
(a) obtaining a first biological sample from a first patient having a DLBCL;
(b) measuring the level of expression of one, two, three, four, five or more of the genes identified in Table 4;
(c) comparing the level of expression of the one, two, three, four, five or more of the genes identified in Table 4 in the first biological sample with the level of expression of the same genes in a second biological sample from a second patient having a DLBCL, wherein the DLBCL in the second patient is clinically insensitive to lenalidomide, or pharmaceutically acceptable salts, solvates or isomers thereof; and
(d) administering lenalidomide, or pharmaceutically acceptable salts, solvates or isomers thereof to the first patient if the one, two, three, four, five or more of the genes in the first biological sample are differentially expressed relative to the level of expression of the one, two, three, four, five or more of the genes in the second biological sample.

11. A method for managing or treating a DLBCL comprising:
(a) obtaining a first biological sample from a first patient having a DLBCL;
(b) measuring the level of expression of one, two, three, four, five or more of the genes identified in Table 1;
(c) comparing the level of expression of the one, two, three, four, five or more of the genes identified in Table 1 in the first biological sample with the level of expression of the same genes in a second biological sample from a second patient having a DLBCL, wherein the DLBCL in the second patient is clinically insensitive to lenalidomide, or pharmaceutically acceptable salts, solvates or isomers thereof; and
(d) administering lenalidomide, or pharmaceutically acceptable salts, solvates or isomers thereof, to the first patient if a higher level of expression of the one, two, three, four, five or more of the genes in the first biological sample is measured relative to the level of expression of the one, two, three, four, five or more of the genes in the second biological sample.

12. A method for managing or treating a DLBCL comprising:
(a) obtaining a first biological sample from a first patient having a DLBCL;
(b) measuring the level of expression of one, two, three, four, five or more of the genes identified in Table 2;
(c) comparing the level of expression of the one, two, three, four, five or more of the genes identified in Table 2 in the first biological sample with the level of expression of the same genes in a second biological sample is from a second patient having a DLBCL, wherein the DLBCL in the second patient is clinically insensitive to lenalidomide, or pharmaceutically acceptable salts, solvates or isomers thereof; and
(d) administering lenalidomide, or pharmaceutically acceptable salts, solvates or isomers thereof, to the first patient if a lower level of expression of the one, two, three, four, five or more of the genes in the first biological sample is measured relative to the level of expression of the one, two, three, four, five or more of the genes in the second biological sample.

13. A method for managing or treating a DLBCL:
(a) obtaining a first biological sample from a first patient having a DLBCL;
(b) measuring the expression of a certain subset of genes set forth in Table 1, 2, 3 or 4, or any combination thereof in the first biological sample,
(c) comparing the gene expression profile of the subset of genes in the first biological sample to (i) the gene expression profile of the subset of genes in tumor samples from patients having DLBCL which are clinically sensitive to lenalidomide, or pharmaceutically acceptable salts, solvates or isomers thereof, and (ii) the gene expression of the subset of genes in tumor samples from patients having DLBCL which are clinically insensitive to lenalidomide, or pharmaceutically acceptable salts, solvates or isomers thereof; and
(d) administering lenalidomide, or pharmaceutically acceptable salts, solvates or isomers thereof, to the first patient if: (i) the gene expression profile for the subset of genes in the first biological sample is similar to the gene expression profile for the subset of genes in tumor samples from patients having DLBCL which are clinically sensitive to lenalidomide, or pharmaceutically acceptable salts, solvates or isomers thereof, and (ii) the gene expression profile for the subset of genes in first biological sample is not similar to the gene expression profile for the subset of genes in tumor samples from patients having DLBCL which are clinically insensitive to lenalidomide, or pharmaceutically acceptable salts, solvates or isomers thereof.

14. A method for managing or treating a DLBCL comprising:
(a) obtaining a first tumor sample from a first patient having a DLBCL;
(b) measuring the proportion of dendritic cells in the first tumor sample;
(c) comparing the proportion of dendritic cells in the first tumor sample with the proportion of dendritic cells in a second tumor sample from a second patient having a DLBCL, wherein the second patient's DLBCL is clinically insensitive to treatment with lenalidomide, or pharmaceutically acceptable salts, solvates or isomers thereof; and
(d) administering lenalidomide, or pharmaceutically acceptable salts, solvates or isomers thereof, to the first patient if a higher proportion of dendritic cells in the first tumor sample is measured relative the proportion of dendritic cells in the second tumor sample.

15. A method for managing or treating a DLBCL comprising:
(a) obtaining a first tumor sample from a first patient having a DLBCL;
(b) measuring the proportion of plasma cells in the first tumor sample;
(c) comparing the proportion of plasma cells in the first tumor sample with the proportion of plasma cells in a second tumor sample from a second patient having a DLBCL, wherein the second patient's DLBCL is clinically insensitive to treatment with lenalidomide, or pharmaceutically acceptable salts, solvates or isomers thereof; and
(d) administering lenalidomide, or pharmaceutically acceptable salts, solvates or isomers thereof, to the first patient if a higher proportion of plasma cells in the first tumor sample is measured relative the proportion of plasma cells in the second tumor sample.

16. A method for managing or treating a DLBCL comprising:
(a) obtaining a first tumor sample from a first patient having a DLBCL;
(b) measuring the proportion of immune cells in the first tumor sample; and
(c) comparing the proportion of the immune cells in the first tumor sample to (i) the proportion of the same immune cells in tumor samples from patients having DLBCL which are clinically sensitive to lenalidomide, or pharmaceutically acceptable salts, solvates or isomers thereof, and (ii) the proportion of the same immune cells in tumor samples from patients having DLBCL which are clinically insensitive to the lenalidomide, or pharmaceutically acceptable salts, solvates or isomers thereof; and
(d) administering lenalidomide, or pharmaceutically acceptable salts, solvates or isomers thereof, to the first patient if the proportion of the immune cells in the first tumor sample is (i) similar to the proportion of the same immune cells in tumor samples from patients having DLBCL which are clinically sensitive to lenalidomide, or pharmaceutically acceptable salts, solvates or isomers thereof, and (ii) not similar to the proportion of the same immune cells in tumor samples from patients having DLBCL which are clinically insensitive to the lenalidomide, or pharmaceutically acceptable salts, solvates or isomers thereof.

17. The method of any one of claims 1 to 16, wherein the DLBCL is refractory.

18. The method of any one of claims 1 to 16, wherein the DLBCL is relapsed in the first patient.

19. The method of any one of claims 1 to 16, wherein the DLBCL is a germinal center B-cell-like subtype.

20. The method of any one of claims 1 to 16, wherein the DLBCL is an activated B-cell-like subtype.
